SEQUENCE LISTING (21 sequences)

SEQ ID NO: 1 - protein, 107 aa, Artificial Sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Artificial Sequence
  Synthetic
IEVMYPPPY

SEQ ID NO: 3 - protein, 7 aa, Artificial Sequence
  Synthetic
PKKKRKV

SEQ ID NO: 4 - protein, 10 aa, Artificial Sequence
  Synthetic
PKKKRKVEAS

SEQ ID NO: 5 - protein, 16 aa, Artificial Sequence
  Synthetic
KRPAATKKAGQAKKKK

SEQ ID NO: 6 - protein, 9 aa, Artificial Sequence
  Synthetic
PAAKRVKLD

SEQ ID NO: 7 - protein, 11 aa, Artificial Sequence
  Synthetic
RQRRNELKRSP

SEQ ID NO: 8 - protein, 38 aa, Artificial Sequence
  Synthetic
NQSSNFGPMKGGNFGGRSSGPYGGGGQYFAKPRNQGGY

SEQ ID NO: 9 - protein, 42 aa, Artificial Sequence
  Synthetic
RMRIZFKNKGKDTAELRRRRVEVSVELRKAKKDEQILKRRNV

SEQ ID NO: 10 - protein, 8 aa, Artificial Sequence
  Synthetic
VSRKRPRP

SEQ ID NO: 11 - protein, 8 aa, Artificial Sequence
  Synthetic
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

SEQ ID NO: 20 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 21 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2024/0043934 A1
(43) Pub. Date: Feb. 8, 2024

(54) PANCREATIC DUCTAL ADENOCARCINOMA SIGNATURES AND USES THEREOF

(71) Applicants: The Broad Institute, Inc., Cambridge, MA (US); Massachusetts Institute of Technology, Cambridge, MA (US); The General Hospital Corporation, Boston, MA (US)

(72) Inventors: Aviv Regev, Cambridge, MA (US); William Hwang, Boston, MA (US); Karthik Jagadeesh, Cambridge, MA (US); Jimmy Guo, Cambridge, MA (US); Tyler Jacks, Cambridge, MA (US); Hannah Hoffman, Cambridge, MA (US)

(21) Appl. No.: 18/021,625

(22) PCT Filed: Aug. 22, 2021
(86) PCT No.: PCT/US2021/047041

§ 371 Date: Feb. 16, 2023

Related U.S. Application Data
(60) Provisional application No. 63/069,035, filed on Aug. 22, 2020.

(51) Int. Cl. C12Q 1/6886

(52) U.S. Cl. CPC C12Q 1/6886; C12Q 2600/112; G01N 2800/7028

(57) ABSTRACT

Described herein are pancreatic ductal adenocarcinoma (PDAC) signatures and methods of detecting the same in a sample from heterogeneity-score a subject. Also described herein, are methods of methods of diagnosing, prognosing, and/or treating PDAC in a subject that can include detecting one or more of the PDAC signatures.

### CROSS-REFERENCE TO RELATED APPLICATIONS

This application claims the benefit of and priority to co-pending U.S. Provisional Patent Application No. 63/069,035, filed on Aug. 22, 2020 entitled “PANCREATIC DUCTAL ADENOCARCINOMA SIGNATURES AND USES THEREOF,” the contents of which is incorporated by reference herein in its entirety.

### SEQUENCE LISTING

This application contains a sequence listing filed in electronic form as an ASCII.txt file entitled BROD-5240WP_ST25.txt, created on Aug. 13, 20201 and having a size of 9,000 bytes. The content of the sequence listing is incorporated herein in its entirety.

### TECHNICAL FIELD

The subject matter disclosed herein is generally directed to signatures, particularly gene expression signatures and tumor microenvironment immune signatures, of pancreatic cancer and uses thereof.

### BACKGROUND

Pancreatic ductal adenocarcinoma (PDAC) is projected to become the second leading cause of cancer death in the United States by 2030^{1,2}. Despite advancements in systemic therapy, many patients cannot receive post-operative chemotherapy and/or radiotherapy (CRT) due to the morbidity often associated with surgery^{3,4}. As such there exists a need for increased resolution of the cell landscape of PDAC and a corresponding development of improved treatments and preventions.

Citation or identification of any document in this application is not an admission that such a document is available as prior art to the present invention.

### SUMMARY

Described in certain example embodiments herein are methods of stratifying pancreatic ductal adenocarcinoma (PDAC) patients into treatment groups and/or prognosing PDAC or treatment outcome and/or survival in a patient comprising: detecting, in one or more PDAC tumor cells from a PDAC tumor, a malignant cell signature, program, or both; a cancer-associated fibroblast (CAF) signature, program, or both; an immune microniche signature, program, or both; a tumor spatial neighborhood; one or more co-expressed receptor-ligand pairs; or any combination thereof; wherein a characteristic regarding a patient's treatment, a patient's response to a treatment, and/or their survival is determined or predicted based on the detection of one or more of the signatures, programs, and/or or states.

In certain example embodiments, the malignant cell signature and/or program comprises a lineage specific expression program selected from: a squamous program, a mesenchymal cytoskeletal program, mesenchymal matrisomal program, a classical progenitor program, a classical activated program, or any combination thereof; a lineage specific expression program selected from: a squamous program, a mesenchymal program, an induced basal-like program, a classical progenitor program, a classical acinar-like program, a classical neuroendocrine-like program, or any combination thereof; a cell state specific expression program selected from: a cycling program, a hypoxic program, TNF-NFkB signaling program, an interferon signaling program, or any combination thereof, a cell state specific expression program selected from: a cycling program, a TNF-NFkB signaling program, or an interferon signaling program, or any combination thereof, a neoadjuvant treated malignant cell expression program; an untreated malignant cell expression program, a cell state expression program selected from: a neuronal-like program, a neuroendocrine like program, a mesenchymal program, a squamoid program, a MYC signaling program, a cycling (G2M) program, a cycling (S) program, or any combination thereof; a lineage specific expression program selected from: an acinar-like program, a classical-like program, a basaloid program, a squamoid program, a mesenchymal program, a neuroendocrine like program, a neuronal like program, or any combination thereof, or any combination thereof.

In certain example embodiments, the CAF signature and/or program (a) comprises a myofibroblast program; a neurotropic program; a secretory program; a mesodermal progenitor program a neuromuscular program; or any combination thereof, (b) comprises a neoadjuvant treated CAF signature and/or program selected from: a neuromuscular program, a secretory program, a neurotropic program, or any combination thereof; (c) comprises an untreated CAF signature and/or program selected from: a mesodermal progenitor program, a myofibroblast program, a neurotropic program, a secretory program, or any combination thereof, or (d) comprises an adhesive expression program, an immunomodulatory expression program, a myofibroblastic progenitor expression program, or a neurotropic expression program.

In certain example embodiments, the tumor spatial neighborhood is a treatment enriched neighborhood, a squamoid-basaloid neighborhood, or a classical neighborhood.

In certain example embodiments, the one or more co-expressed receptor-ligand pairs is selected from: an Epithelial compartment—CAF compartment pair; an Epithelial compartment—Immune compartment pair; a CAF compartment and Immune compartment pair; or any combination thereof.

In certain example embodiments, the method comprises, detecting, in one or more a PDAC tumor cells, an untreated tumor malignant cell signature and/or program and an untreated CAF signature and/or program, wherein the untreated tumor malignant cell signature and/or program comprises a lineage specific expression program selected from: a squamous program, a mesenchymal cytoskeletal program, mesenchymal matrisomal program, a classical progenitor program, a classical activated program, or any combination thereof, and the untreated tumor CAF signature and/or program is selected from: a mesodermal progenitor program, a myofibroblast program, a neurotropic program, a secretory program, or any combination thereof.

In certain example embodiments, the method further comprises determining a tumor heterogeneity score for the PDAC tumor, wherein the tumor heterogeneity score is calculated by determining a number of highly expressed programs in the one or more PDAC cells.

In certain example embodiments, the method further comprises assigning the PDAC tumor to a single malignant class and to a single CAF class, wherein the malignant class is selected from A0, A1, A2, S0, S1, S2, C0, C1, C2, M0, M1, M2, P0, P1, or P2, and wherein the CAF class is selected from S0, S1, NO, N1, M0, M1, P0, or P1.

In certain example embodiments, the PDAC tumor is assigned to a combined risk class that integrates the malignant risk group and CAF risk group class and is selected from: a low combined risk group, a low-intermediate combined risk group, a high-intermediate risk group, or a high combined risk group, wherein a PDAC tumor in a low malignant risk group and in a low CAF risk group is classified into the low combined risk group; a PDAC tumor in a high malignant risk and in a high CAF risk is classified into the high combined risk group; a PDAC tumor in an intermediate malignant risk group or in an intermediate CAF risk and in a high malignant risk or in a high CAF risk is classified into the high-intermediate combined risk group; and a PDAC tumor in a low malignant risk group and in a high CAF risk group, a PDAC tumor in a high malignant risk group and in a low CAF risk group, a PDAC tumor in a low malignant risk group and in a low CAF risk group, a PDAC tumor in an intermediate malignant risk group and in an intermediate CAF risk group, a PDAC tumor in a low malignant risk group and in an intermediate CAF risk group is classified into the low-intermediate combined risk group.

In certain example embodiments, a subject with a PDAC tumor in low combined risk group has the greatest likelihood of longest survival.

In certain example embodiments, a subject having a classical-like malignant expression program has the greatest likelihood of time to progression and longest survival.

In certain example embodiments, a subject having an immunomodulatory CAF expression program has the greatest likelihood of time to progression.

In certain example embodiments, a subject having a neuronal like malignant expression program or a malignant squamoid expression program has the greatest likelihood of least time to progression.

In certain example embodiments, a subject having an adhesive CAF expression program has the greatest likelihood of shortest survival.

In certain example embodiments, the malignant cell signature comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

In certain example embodiments, the CAF cell signature comprises one or more biomarkers, expression programs, biologic programs, receptor-ligand interactions, cell state distribution, cell type distribution, or any combination thereof as in any of FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

In certain example embodiments, the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39 and Table 7, or any combination thereof.

In certain example embodiments, there is a greater likelihood of longer survival when a predominant mesenchymal matrisomal and/or a classical progenitor malignant program is detected as compared to detection of a primary classical activated, a squamous, or a mesenchymal cytoskeletal program.

In certain example embodiments, there is a greater likelihood of longer survival when a CAF secretory or neurotropic program is detected as compared to detection of a myofibroblast or mesodermal progenitor program is detected.

In certain example embodiments, the PDAC patient had or is concurrently receiving a neoadjuvant therapy.

In certain example embodiments, detecting comprises a single cell RNA sequencing technique.

In certain example embodiments, detecting comprises a single-nucleus RNA sequencing technique.

In certain example embodiments, the single-nucleus RNA sequencing technique is optimized for pancreatic tissue.

In certain example embodiments, the single-nucleus RNA sequencing technique is optimized for frozen samples.

In certain example embodiments, the single-nucleus RNA sequencing technique comprises screening a sample for an RNA integrity number and performing single nucleus RNA sequencing only on samples with an RNA integrity number of 6 or more.

In certain example embodiments, detecting comprises a spatially-resolved transcriptomics technique.

Described in certain example embodiments herein are methods treating pancreatic ductal adenocarcinoma (PDAC) in a subject in need thereof comprising: preventing a shift in the state of a malignant cell from a classical progenitor state to a basal-like state or a terminally-differentiated state; modulating a cell state of a malignant cell from a basal-like state or a terminally-differentiated state to a classical progenitor state; inhibiting, preventing, or modulating expression of a neuronal like expression program in a malignant cells; inhibiting, preventing expression or modulating expression of a malignant squamoid expression program in a malignant cell, inhibiting, preventing, or modulating expression of an adhesive CAF expression program in a CAF cell; or any combination thereof.

In certain example embodiments, the subject has had neoadjuvant therapy; is concurrently receiving or undergoing neoadjuvant therapy; or the subject has not had neoadjuvant therapy.

In some embodiments, a malignant cell state is characterized by a malignant cell signature comprising: a lineage specific expression program selected from a squamous program, a mesenchymal cytoskeletal program, mesenchymal matrisomal program, a classical progenitor program, or a classical activated program; a lineage specific expression program selected from a squamous program, a mesenchymal program, an induced basal-like program, a classical progenitor program, a classical acinar-like program, and a classical neuroendocrine-like program; a cell state specific expression program selected from a cycling program, a hypoxic program, TNF-NFkB signaling program, or an interferon signaling program; a cell state specific expression program selected from a cycling program, a TNF-NFkB signaling program, or an interferon signaling program; a neoadjuvant treated malignant cell expression program; an untreated malignant cell expression program; a basal-like malignant cell expression program; a classic-like malignant cell expression program; an immune microniche signature; or a combination thereof.

In certain example embodiments, the malignant cell signature comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

In certain example embodiments, modulating the cell state comprises reducing the distance in gene expression space between the basal-like malignant cell state and the classic-like malignant cell states.

In certain example embodiments, the gene expression spaces comprises 10 or more genes, 20 or more genes, 30 or more genes, 40 or more genes, 50 or more genes, 100 or more genes, 500 or more genes, or 1000 or more genes.

In certain example embodiments, the distance is measured by a Euclidean distance, Pearson coefficient, Spearman coefficient, or combination thereof.

In certain example embodiments, modulation comprises increasing or decreasing expression of one or more genes, gene expression cassettes, or gene expression signatures.

In certain example embodiments, modulating or preventing comprises administering a modulating agent to the subject.

In certain example embodiments, the modulating agent comprises a therapeutic antibody or fragment thereof, antibody-like protein scaffold, aptamer, polypeptide, a polynucleotide, a genetic modifying agent or system, a small molecule therapeutic, a chemotherapeutic, small molecule degrader, inhibitor, an immunomodulator, or a combination thereof.

Described in certain example embodiments herein are methods of screening for one or more agents capable of modulating a PDAC malignant cell state comprising: contacting a cell population comprising PDAC malignant cells having an initial cell state with a test modulating agent or library of modulating agents; determining a fraction of malignant cells having a desired cell state and an undesired cell state; selecting modulating agents that shift the initial PDAC malignant cell state to a desired cell state or prevent the initial PDAC malignant cell state to shift from a desired initial state, such that the fraction of PDAC malignant cells in the cell population having a desired cell state is above a set cutoff limit.

In certain example embodiments, the desired PDAC malignant cell state is a classic progenitor cell state or a mesenchymal matrisomal cell state.

In certain example embodiments, the cell population is obtained from a subject to be treated.

Described in certain example embodiments herein are methods of treating a subject having pancreatic ductal adenocarcinoma (PDAC), the method comprising: administering a neoadjuvant therapy to the subject; and administering a PDAC malignant cell modulating agent, an immune modulator, a CAF modulating agent, an apoptosis inhibitor, a myeloid cell agonist, a TGFbeta modulator, a CXCR4 inhibitor, a HER2 inhibitor, or any combination thereof to the subject.

Described in certain example embodiments herein are methods treating a subject having PDAC, the method comprising: detecting, in one or more PDAC tumor cells, a malignant cell signature, program, or both; a cancer-associated fibroblast (CAF) signature, program, or both; an immune microniche signature, program, or both; a tumor spatial neighborhood, one or more co-expressed receptor-ligand pairs, or any combination thereof, and administering or applying a PDAC treatment to the subject in need thereof, wherein the treatment is optionally a tumor resection, a chemotherapy, a radiation therapy, a neoadjuvant, a malignant cell signature and/or program modulating agent, a BCL-2 inhibitor, a tyrosine kinase inhibitor, a TGFbeta modulator, a myeloid cell agonist, a CXCR4 inhibitor, a HER2 inhibitor, or any combination thereof.

In certain example embodiments, the malignant cell signature and/or program comprises a lineage specific expression program selected from: a squamous program, a mesenchymal cytoskeletal program, mesenchymal matrisomal program; a classical progenitor program, a classical activated program, or any combination thereof lineage specific expression program selected from: a squamous program, a mesenchymal program, an induced basal-like program, a classical progenitor program, a classical acinar-like program, a classical neuroendocrine-like program, or any combination thereof; a cell state specific expression program selected from: a cycling program, a hypoxic program, TNF-NFkB signaling program, an interferon signaling program, or any combination thereof, a cell state specific expression program selected from: a cycling program, a TNF-NFkB signaling program, or an interferon signaling program, or any combination thereof, a neoadjuvant treated malignant cell expression program; an untreated malignant cell expression program; a cell state expression program selected from: a neuronal-like program, a neuroendocrine like program, a mesenchymal program, a squamoid program, a MYC signaling program, a cycling (G2M) program, a cycling (S) program, or any combination thereof; a lineage specific expression program selected from: an acinar-like program, a classical-like program, a basaloid program, a squamoid program, a mesenchymal program, a neuroendocrine like program, a neuronal like program, or any combination thereof, or any combination thereof.

In certain example embodiments, the CAF signature and/or program comprises a myofibroblast program; a neurotropic program; a secretory program; a mesodermal progenitor program a neuromuscular program; or any combination thereof, comprises a neoadjuvant treated CAF signature and/or program selected from: a neuromuscular program, a secretory program, a neurotropic program, or any combination thereof, comprises an untreated CAF signature and/or program selected from: a mesodermal progenitor program, a myofibroblast program, a neurotropic program, a secretory program, or any combination thereof, or comprises an adhesive expression program, an immunomodulatory expression program, a myofibroblastic progenitor expression program, or a neurotropic expression program.

In certain example embodiments, the method comprises, detecting, in one or more a PDAC tumor cells, an untreated tumor malignant cell signature and/or program and an untreated CAF signature and/or program, wherein the untreated tumor malignant cell signature and/or program comprises a lineage specific expression program selected from: a squamous program, a mesenchymal cytoskeletal program, mesenchymal matrisomal program, a classical progenitor program, a classical activated program, or any combination thereof, and the untreated tumor CAF signature and/or program is selected from: a mesodermal progenitor program, a myofibroblast program, a neurotropic program, a secretory program, or any combination thereof.

In certain example embodiments, the method further comprises determining a tumor heterogeneity score for the PDAC tumor, wherein the tumor heterogeneity score is calculated by determining a number of highly expressed programs in the one or more PDAC cells.

In certain example embodiments, the method further comprises assigning the PDAC tumor to a single malignant class and to a single CAF class, wherein the malignant class is selected from A0, A1, A2, S0, S1, S2, C0, C1, C2, M0, M1, M2, P0, P1, or P2, and wherein the CAF class is selected from S0, S1, NO, N1, M0, M1, P0, or P1.

In certain example embodiments, the PDAC tumor is assigned to a combined risk class that integrates the malignant risk group and CAF risk group class and is selected from: a low combined risk group, a low-intermediate combined risk group, a high-intermediate risk group, or a high combined risk group, wherein a PDAC tumor in a low malignant risk group and in a low CAF risk group is classified into the low combined risk group; a PDAC tumor in a high malignant risk and in a high CAF risk is classified into the high combined risk group; a PDAC tumor in an intermediate malignant risk group or in an intermediate CAF risk and in a high malignant risk or in a high CAF risk is classified into the high-intermediate combined risk group; and a PDAC tumor in a low malignant risk group and in a high CAF risk group, a PDAC tumor in a high malignant risk group and in a low CAF risk group, a PDAC tumor in a low malignant risk group and in a low CAF risk group, a PDAC tumor in an intermediate malignant risk group and in an intermediate CAF risk group, a PDAC tumor in a low malignant risk group and in an intermediate CAF risk group is classified into the low-intermediate combined risk group.

In certain example embodiments, a subject with a PDAC tumor in low combined risk group has the greatest likelihood of longest survival.

In certain example embodiments, a subject having a classical-like malignant expression program has the greatest likelihood of time to progression and longest survival.

In certain example embodiments, a subject having an immunomodulatory CAF expression program has the greatest likelihood of time to progression.

In certain example embodiments, a subject having a neuronal like malignant expression program or a malignant squamoid expression program has the greatest likelihood of least time to progression.

In certain example embodiments, a subject having an adhesive CAF expression program has the greatest likelihood of shortest survival.

In certain example embodiments, the tumor spatial neighborhood is a treatment enriched neighborhood, a squamoid-basaloid neighborhood, or a classical neighborhood.

In certain example embodiments, the one or more co-expressed receptor-ligand pairs is selected from an Epithelial compartment—CAF compartment pair; an Epithelial compartment—Immune compartment; a CAF compartment and Immune compartment pair; or any combination thereof.

In certain example embodiments, the malignant cell signature comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

In certain example embodiments, the CAF cell signature one or more biomarkers, expression programs, biologic programs, receptor-ligand interactions, cell state distribution, cell type distribution, or any combination thereof FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

In certain example embodiments the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39, and Table 7, or any combination thereof.

In certain example embodiments, the PDAC treatment comprises a neoadjuvant therapy.

In certain example embodiments, the PDAC treatment comprises preventing a shift in the state of a malignant cell from a classical progenitor state to a basal-like state or a terminally-differentiated state; modulating a cell state of a malignant cell from a basal-like state or a terminally-differentiated state to a classical progenitor state; inhibiting, preventing, or modulating expression of a neuronal like expression program in a malignant cells; inhibiting, preventing expression or modulating expression of a malignant squamoid expression program in a malignant cell; inhibiting, preventing, or modulating expression of an adhesive CAF expression program in a CAF cell; or any combination thereof.

In certain example embodiments, the PDAC treatment is a PDAC signature modulating agent.

In certain example embodiments, the PDAC modulating agent is selected by performing a modulating agent screening method as described in greater detail above and elsewhere herein.

In certain example embodiments, the subject has had neoadjuvant therapy, is concurrently receiving or undergoing neoadjuvant therapy; or the subject has not had neoadjuvant therapy.

In certain example embodiments, the subject has had a PDAC tumor resected prior to administration.

In certain example embodiments, the subject has not had a PDAC tumor resected prior to administration.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1D—Single-nucleus RNA-seq of PDAC captures representative cell type distributions across malignant, epithelial, immune and stromal compartments. FIG. 1A, Experimental workflow of human PDAC tumors for snRNA-seq, Multiplex Ion Beam Imaging (MIBI), and/or digital spatial profiling (NanoString GeoMx). FIG. 1B,1C, snRNA-seq captures diverse malignant, epithelial, immune and stromal cell subsets. FIG. 1B, Mean expression (greyscale bar) selected marker genes (columns) across annotated cell subsets (rows) of different compartments (labels, left) from untreated (left) and treated (right) tumors. FIG. 1C, UMAP embedding of single nucleus profiles (dots) from untreated (top) and treated (bottom) tumors shaded by post hoc cell type annotations (greyscale legend). Insets: UMAP re-embedding of single nucleus profiles from specific subsets of interest. FIG. 1D, snRNA-seq captures representative cell type distributions compared to in situ assessment. Top: Proportion of cells (y axis) in each of the four major compartments (greyscale legend) as estimated by snRNA-seq or MIBI (x axis) in aggregate across all untreated (left; n=5) or treated (right, n=2) tumors. Bottom: Representative MIBI images and segmentation showing staining with antibodies against cytokeratin (shaded), vimentin (shaded), CD45 (shaded), CD31 (shaded) and double-stranded DNA (shaded).

FIG. 2A-2D—Neoadjuvant chemoradiotherapy remodels cellular subsets, programs and interactions in compartment-specific manner. FIG. 2A, CRT remodels cell type composition across compartments. Proportions (y axis) of cell subsets (x axis) in untreated (n=15) vs. treated (n=11) patients of all cells in the tumor (left) and of immune cell only (right). * p<0.05, Fisher's exact test. FIG. 2B, Treatment impact on putative cell interactions. Selected putative interactions between cell subsets (y-axis, greyscale) differentially impacted by treatment status based on expression of the receptor (left) in one cell subset, and its cognate ligand (right) in another (Methods), distinguishing the expression level (greyscale bar) and proportion of expressing cells (x axis) in untreated (solid borders) and treated (dashed borders) tumors. * p<0.05, t†p<0.1; Fisher's exact test or chi-square test with Yates correction. FIG. 2C, Interferon signaling and basal-like genes induced in malignant cells from CRT-treated tumors. Top: differential expression (log 2(fold-change), x axis) and its significance (-log 10(adjusted p-value), y axis, DESeq2 R package) of genes in malignant cells between treated and untreated tumors. Names of selected significant genes are marked. Bottom: Gene Set Enrichment Analysis (GSEA^{143,149,150}) terms (y axis) ranked by increasing significance (-log₁₀(FDR q-value)) of enrichment for induction in treated tumors. FIG. 2D, Summary of compartment specific effects of CRT.

FIG. 3A-3H—Refined molecular taxonomy of PDAC improves clinical prognostication and highlights a shift from classical-like to basal-like and differentiated-like programs in malignant cells. FIG. 3A,3B, A consensus NMF (cNMF) expression program dictionary in untreated and treated tumors. UMAPs of single nucleus profiles (dots) from untreated (FIG. 3A) and treated (FIG. 3B) tumors, shaded by patient (top left) or by the score derived for each cell-cNMF program pair (Methods). FIG. 3C, Shift towards basal-like programs in CRT treated tumors. Proportion of malignant cells primarily expressing each lineage-specific malignant cell cNMF program within untreated (left) and treated (right) tumors. FIG. 3D, Refined PDAC molecular taxonomy with proposed model of transcriptional programs and their relationships. FIG. 3E-3G, Refined molecular taxonomy of malignant and fibroblast cells has prognostic value. FIG. 3E, Tumor assignment to risk categories defined by primary snRNA-seq program (first letter) and heterogeneity score (second number) for malignant cells (rows) and fibroblasts (columns) separately (red and blue shades, Methods) and by their combination (purple shades, Methods) for bulk RNA-seq classification of patients with untreated respectable PDAC (n=307). The number of tumors in each stratification is listed. For malignant programs: P=classical progenitor, A=classical activated, M=mesenchymal matrisomal, C=mesenchymal cytoskeletal, S=squamous. For fibroblast programs: N=neurotropic, S=secretory, P=mesodermal progenitor, M=myofibroblast. For heterogeneity score: 0=fewer than two highly-expressed programs, 1=two highly-expressed programs (malignant) or two or more high-expressed programs (fibroblast), and 2=three or more highly-expressed programs (malignant). FIG. 3F-3H, Kaplan-Meier survival analysis of bulk RNA-seq cohort (n=307) based on risk groups from malignant (FIG. 3F), fibroblast (FIG. 3G) and combined (FIG. 311) strata, as defined in (FIG. 3E). Survival distributions are compared by the log-rank test. Number of patients at risk at the beginning of each time interval is shown in the table.

FIG. 4A-4F—Basal-like and classical-like programs are associated with spatial niches with distinct quantity and quality of immune infiltration. FIG. 4A, 4B, Definition of distinct regions of interest (ROIs) with GeoMx DSP. FIG. 4A, Immunofluorescence images (left, GeoMx DSP) and matched consecutive hematoxylin and eosin (H&E)-stained FFPE sections (5 μm thickness, right) of three patients (labeled, top), showing the selected ROIs (circles). FIG. 4B, Top: GeoMx DSP immunofluorescence images with selected ROIs (circles, 600 μm diameter) representing three classes of epithelial niches infiltrated with either both immune cells and CAFs (left), only with CAFs (middle), or only with immune cells (right). Bottom: Segmentation masks on the ROIs, used to enrich for the epithelial, CAF, and immune compartments. Gray=SYTO13 (nuclear stain), green=anti-panCK, magenta=anti-CD45, cyan=anti-aSMA. FIG. 4C, Increased basal-like programs in treated tumors in situ. Aggregate basal-like and classical-like signature scores (y axis) for each epithelial area of interest (AOI) from untreated (grey) and treated (black) tumors, rank-ordered by score as determined from the GeoMx Cancer Transcriptome Atlas (CTA). FIG. 4D, GeoMx Whole Transcriptome Assay (WTA) also detected intra-tumoral heterogeneity in untreated malignant and CAF programs. Magnitude of expression (amplitude) for each gene (position in circle plot) in each malignant or CAF program (segment in circle plot, schematic on left) in three ROIs selected and segmented as for the CTA in (b) on specimen PDAC_U_7. FIG. 4E, Basal-like and classical-like malignant programs associate with immune niches with distinct characteristics. Expression (z-score of normalized counts across AOIs; greyscale bar) of immune cell signature genes (rows) from diverse cell types and signatures (FIG. 4E, greyscale legend and left bar) across immune AOIs (columns) from untreated (grey) and treated (black) tumors in ROIs with either basal-like (shaded) or classical-like (shaded) malignant cells. HLA=human leukocyte antigen module, ISG=interferon-stimulated gene module. FIG. 4F, Expression (z-score of the normalized counts across AOIs; greyscale bar) of subtype-associated immune cell type genes (rows) across immune AOIs (columns) from untreated (grey) and treated (black) tumors in ROIs with either basal-like (shaded) or classical-like (shaded) malignant cells.

FIG. 5A-5C—Cell type composition across PDAC tumors. FIG. 5A, UMAP embeddings of single nucleus profiles (dots) from individual tumors (panels) from untreated (left) and treated (right) patients shaded by post hoc cell type annotations (greyscale legend).

FIG. 5B,5C. Cell type compositions across tumors. Proportion of nuclei (y axis) of each cell type (greyscale legend) in each tumor (x axis) from untreated (left) and treated (right) patients, out of all cells (FIG. 5B) or when considering only non-malignant cells (FIG. 5C).

FIG. 6A-6B—Inferred CNAs recover common aberrations based on PDAC genome studies. FIG. 6A, Example inferCNV analysis. Inferred amplifications (shaded) and deletions (shaded) based on expression (greyscale bar) in 100-gene window in each locus (columns) from each cell (rows) labeled by its annotated expression type (greyscale code) in reference cells from matched adjacent normal tissue (top) and cells from the tumor (bottom). FIG. 6B, Inferred CNA frequencies in the cohort agree with PDAC genome studies. Frequency (y axis) of CNAs on each chromosome arm (x axis) as inferred across the patients in the cohort (light shaded bars) and from genome analysis of PDAC (dark shaded bars) and prostate adenocarcinoma (PRAD) (grey bars) from TCGA cohorts.

FIG. 7—snRNA-seq captures representative cell types distributions compared to in situ assessment by MIBI. Proportion of cells (y axis) in each of the four major compartments (greyscale legend) as estimated by snRNA-seq or MIBI (x axis) in each untreated (top; n=5) or treated (bottom; n=2) tumor measured by MIBI (2-3 fields of view per slide).

FIG. 8A-8B—CRT remodels cell type composition across compartments. Proportions (y axis) of cell subsets (x axis) in untreated (n=15) vs. treated (n=11) patients out of all non-malignant cells in the tumor (FIG. 8A) or out of all stromal cells only (FIG. 8B). * p<0.05, Fisher's exact test.

FIG. 9A-9D—Treatment impact of gene expression in different cell subsets. FIGS. 9A-9C, Differential expression (log₂(fold-change), x axis) and its significance (-log₁₀(adjusted p-value), y axis, DESeq2 R package) between treated and untreated tumors (FIG. 9A, CD8+T lymphocytes, FIG. 9B, macrophages, FIG. 9C, malignant cells omitting two treated tumors with germline BRCA2 mutations (PDAC_T_1,2)) or (FIG. 9D) of genes in malignant cells from treated tumors with high (>10%, PDAC_T_5,7,10,11) vs. low (PDAC_T_1,2,3,4,6,8,9) residual neoplastic content. Names of selected significant genes are marked.

FIG. 10—Bulk-derived tumor subtype signatures across single nuclei in the PDAC cohort. UMAP embeddings of single nucleus profiles (dots) from all tumor nuclei (top two panels) or only malignant cells (bottom two panels) separately for untreated and treated patients shaded by expression score (greyscale bar, Methods) of signatures derived from the Bailey¹⁰, Collisson⁷, Moffitt⁹, and COMPASS/PanCuRx⁷⁸ studies.

FIG. 11—cNMF program distributions across malignant cells in individual tumors. Proportion of cells (y axis) assigned with highest scoring program (greyscale legend) in individual tumors (x axis) in the untreated (left) and treated (right) groups.

FIG. 12—Association between basal-like and interferon, TNF-NFkB programs. Normalized correlation (greyscale bar) of the gene weights for each cNMF program (rows, columns) in untreated (left) or treated (right) tumors.

FIG. 13A-13G—Survival analysis of bulk RNA-seq PDAC cohort based on malignant cell and fibroblast programs and heterogeneity score. FIG. 13A-13G, Kaplan-Meier survival analyses of PDAC cohort (n=307) from TCGA¹¹ and PanCuRx⁷⁸, stratified by primary malignant program (FIG. 13A), malignant heterogeneity score (FIG. 13B), combined primary malignant program and heterogeneity score (FIG. 13C), primary fibroblast program (FIG. 13D), fibroblast heterogeneity score (FIG. 13E), combined primary fibroblast program and heterogeneity score (FIG. 13F), and combined primary malignant program and heterogeneity score with combined primary fibroblast program and heterogeneity score (FIG. 13G). Survival distributions for each patient strata were compared using the log-rank test.

FIG. 14—Previous CAF subset signatures across single fibroblast profiles in the PDAC cohort. UMAP embeddings of single nucleus profiles (dots) from fibroblast nuclei from untreated (top) and treated (bottom) patients shaded by expression score (greyscale bar, Methods) of three signatures previously reported by Tuveson and colleagues⁵¹.

FIG. 15A-15D—Differences in fibroblast gene expression, composition and programs in treated tumors. FIG. 15A, Cell intrinsic expression differences in fibroblasts from CRT-treated tumors. Left: differential expression (log₂(fold-change), x axis) and its significance (−log₁₀(adjusted p-value), y axis, DESeq2 R package) of genes in fibroblasts between treated and untreated tumors. Names of selected significant genes are marked. Right: GSEA^{143,149,150} terms (y axis) ranked by increasing significance (-log₁₀(FDR q-value)) of enrichment in treated tumors. FIG. 15B, cNMF expression program dictionary in fibroblasts from untreated and treated tumors. UMAP embeddings of single nucleus profiles (dots) from untreated (top) and treated (bottom) tumors, shaded by patient (left panel) or by the score derived for each cell-cNMF program pair (greyscale bar, Methods). FIG. 15C, Normalized correlation (greyscale bar) of the gene weights for each cNMF program (rows, columns) in untreated (top) or treated (bottom) tumors. FIG. 15D, Higher proportion of myofibroblast and neuromuscular programs in CRT treated tumors. Proportion of fibroblasts primarily expressing each fibroblast cNMF program within untreated (left) and treated (right) tumors, in aggregate across all tumors (top) or in individual tumors (bottom, x axis).

FIG. 16A-16E—Assessing PDAC programs by digital spatial profiling. FIG. 16A, Experimental workflow for digital spatial profiling on the GeoMx platform (NanoString). FIG. 16B, Spatial resolution of cell types across ROIs and AOIs. Expression (z-score of normalized counts across AOIs; purple/yellow greyscale bar) of signature genes (rows) from diverse cell types (greyscale legend (4) and left bar) across AOIs (columns, greyscale legend and horizontal bar (3)) profiled by 1,412-gene cancer transcriptome atlas or CTA, capturing epithelial (green), fibroblasts (blue) and immune (red) cells, from ROIs characterized by presence or absence of immune (greyscale legend and horizontal bar (1)) and fibroblast (greyscale legend and horizontal bar (2)) infiltration. Both columns and rows are clustered by unsupervised hierarchical clustering. FIG. 16C, Box-plots comparing mean normalized gene expression by cluster for basal-like vs. classical-like epithelial AOIs. * p<0.05, Student's t-test. FIG. 16D, Coverage of PDAC snRNA-seq programs by CTA and WTA digital spatial profiling. Number of genes (y axis) from each untreated malignant cell program (x axis) captured by CTA only (white), WTA only (black), or both (grey). FIG. 16E, Impact of gene panel on program scores. Spearman correlation coefficient (p) between the scores for different untreated malignant programs (x axis) obtained with WTA using the full gene panel vs. the gene subset shared with the CTA assay.

FIGS. 17A-17G—Single-nucleus RNA-seq of untreated and treated PDAC captures representative diversity of cell types including putative ADM intermediate. FIG. 17A, Experimental workflow of human PDAC tumors for snRNA-seq, Multiplex Ion Beam Imaging (MIBI), and digital spatial profiling (NanoString GeoMx). FIG. 17B, snRNA-seq captures diverse malignant, epithelial, immune and other stromal cell subsets. Mean expression (greyscale bar) of selected marker genes (columns) across annotated cell subsets (rows) of different compartments (labels, left). FIG. 17C, Distinctions between patients or treatment status. UMAP embedding of single nucleus profiles (dots) of PDAC tumors shaded by patient ID (greyscale legend, left) or treatment status (right). FIG. 17D, Cell subsets in each compartment. UMAP embeddings of single nucleus profiles of all cells (left, as in FIG. 17C) or in each compartment (right insets) shaded by post hoc cell type annotations (color legend).

FIGS. 17E, 17F, Inferred differentiation states in pre-malignant and malignant cells. FIG. 17E, Proportion of cells (dot size) with non-zero expression of gene set HALLMARK_KRAS_SIGNALING_UP in each epithelial cell subset and normalized mean expression (dot color) in expressing cells. FIG. 17F, Partition-based graph abstraction (PAGA) of an inferred pseudotemporal trajectory among epithelial cell subsets (nodes). FIG. 17G, snRNA-seq captures representative cell types distributions compared to in situ assessment. Top: Representative MIBI images and segmentation showing staining with antibodies against cytokeratin (green, represented in greyscale), vimentin (blue, represented in greyscale), CD45 (red, represented in greyscale), CD31 (purple, represented in greyscale) and double-stranded DNA (gray, represented in greyscale). Bottom: Proportion of cells (y axis) in each of the four major compartments (left panels, greyscale legend) or in each of the immune subsets (right panels, greyscale legend) as estimated by snRNA-seq or MIBI (x axis) in aggregate across all untreated (two left bars; n=5) or treated (two right bars; n=2) tumors.

FIGS. 18A-18E—Refined molecular taxonomy of PDAC reveals treatment-associated differences and identifies a novel neuronal-like malignant program enriched after treatment and associated with poor clinical outcomes. FIG. 18A, Remodeling of tumor composition by treatment. Proportions (y axis) of each cell subset (x-axis) among all (left) or immune (right) nuclei. (* p<0.05; ** p<0.01; * ** p<0.001, Pairwise comparisons were performed using the Mann-Whitney U test). FIG. 18B, Expression program dictionary in malignant cells and CAFs. UMAPs of single nucleus profiles (dots) of malignant cells (top and middle) and CAFs (bottom) from all tumors, colored by patient (bottom right, malignant; bottom left, CAF) or by the normalized expression score of each program (Methods). FIG. 18C, Distinctions between the neuronal-like and neuroendocrine-like programs. Overlap of each gene set (shaded pie charts) with the neuronal-like (green, represented in greyscale) and neuroendocrine-like (red, represented in greyscale) programs. Beige circles depict clusters of related gene sets. Edges represent overlaps between distinct gene sets based on an overlap coefficient threshold (>0.85, Cytoscape). FIG. 18D, Malignant cell and CAF programs associated with treatment status. Mean normalized program expression (y axis) of malignant cell state (top), malignant cell lineage (middle), and CAF (bottom) programs (x axis) in untreated (n=18), CRT (n=14), and CRTL (n=5) tumors. * p<0.05, ** p<0.01, * ** p<0.001, **** p<0.0001, Mann-Whitney U test. FIG. 18E, Program association with clinical features. Hazards ratio (middle) and p-value (left) for each variable (clinicopathologic and program expression score in bulk RNA-Seq, rows) from TCGA and PanCuRx with untreated, resected primary PDAC.

FIGS. 19A-19D—Spatial mapping of malignant programs, CAF programs and immune cell composition in untreated and treated PDAC tumors reveals three distinct multicellular neighborhoods. FIG. 19A, Whole Transcriptome Digital Spatial Profiling (WTA DSP). Left: Representative hematoxylin and eosin (H&E)-stained FFPE sections (5 μm thickness, left) and immunofluorescence image (GeoMx DSP, right) of consecutive sections from the same tumor FFPE, showing selected regions of interest (ROIs, circles). Gray=SYTO13 (nuclear stain), green=anti-panCK, magenta=anti-CD45, cyan=anti-aSMA. Right: Example ROI (circle, 600 μm diameter) with segmentation masks used to enrich for the epithelial, CAF, and immune compartments and percent of total segment area occupied by each compartment. FIG. 19B, Higher variation across tumors that within tumor ROIs. Left: Normalized expression (grey scale) of malignant cell (top heat map) and fibroblast (bottom heat map) programs (rows) in each AOI (columns) across patients (top greyscale bar, greyscale legend) and treatment status (bottom greyscale bar, greyscale greyscale legend). Right: Program expression variation between patients (y axis, interquartile range (IQR) of the mean program score for each tumor) and within patients (x axis, mean of IQR across all ROIs within a tumor). Dotted line x=y. FIGS. 19C-19D, Three multicellular neighborhoods with distinct malignant, CAF, and immune features. FIG. 19C, Unsupervised hierarchical clustering of whole transcriptome DSP ROI-based feature correlation matrix using Pearson correlation coefficients (greyscale bar). FIG. 19D, Schematic of key features of each multicellular neighborhood as defined in FIG. 19C.

FIGS. 20A-20C—Spatially-defined associations of malignant programs and intercellular receptor-ligand interactions as a function of treatment. FIG. 20A, Fold change (color bar) of inferred immune subset proportions (rows) between the top quartile scoring ROIs and the bottom quartile scoring ROIs for each malignant (columns; left) or fibroblast (columns; right) program. FIG. 20B, Cell intrinsic and clinical characteristics and spatial associations for malignant lineage programs (columns). FIG. 20C, Spatially correlated receptor-ligand pairs across compartments. Spearman rank correlation coefficient of expression of receptor-ligand pairs (gray dots) across paired epithelial:CAF (left) epithelial:immune (middle) or CAF:immune (right) segments within the same ROI across all ROIs in CRT-treated (y axis) or untreated (x axis) tumors. Selected receptor-ligand pairs that were differentially correlated in CRT-treated or untreated tumors are labeled and colored based on the segment expressing the ligand (color legend). Solid line: x=y.

FIGS. 21A-21B—Cell type composition across PDAC tumors. FIG. 21A, UMAP embeddings of single nucleus profiles (dots) from individual tumors (panels) from untreated (left) and treated (right) patients shaded by post hoc cell type annotations (greyscale legend).

FIG. 21B, Cell type distributions across tumors. Proportions (y axis) of cell subsets (greyscale legend) across untreated (n=18) (left) vs. treated (n=25) tumors (right) either with (top) or without (bottom) malignant cells. Treated patients are further classified by specific treatment type.

FIGS. 22A-22B—Inferred CNAs recapitulate prior PDAC genomic studies. FIG. 22A, Example inferCNV analysis of the epithelial subset from a study specimen. Inferred amplifications (red, represented in greyscale) and deletions (blue, represented in greyscale) based on expression (greyscale bar) of sliding 100-gene window in each chromosomal locus (columns) from each cell (rows) labeled by its annotated cell type (shading code). FIG. 22B, Inferred CNA frequencies in the snRNA-seq cohort have similar distribution as those derived from TCGA genomic study¹⁰. Frequency (y axis) of CNAs on each chromosome arm (x axis) as inferred across the patients in the snRNA-seq cohort (light green bars, represented in greyscale) and from genome analysis of PDAC (dark green bars, represented in greyscale) from the TCGA cohort.

FIG. 23—snRNA-seq captures a greater diversity and abundance of cell types relative to prior single-cell approaches. Number of nuclei/cells per untreated tumor that passed quality control filters (y axis) in our study (n=18) vs. Peng et al. study (n=24)⁵⁶ (grayscale legend), in total (left) and broken out by cell type (right). * p<0.05, ** p<0.01, *** p<0.001, **** p<0.0001, Mann-Whitney U test.

FIG. 24—snRNA-seq captures representative cell type distributions compared to in situ assessment by MIBI. Proportion of cells (y axis) in each of the four major compartments (color legend, top) or immune cell subsets (greyscale legend, bottom) as estimated by snRNA-seq or MIBI (x axis) in each matched untreated (left; n=5) or treated (right; n=2) tumor.

FIG. 25—Treatment associated with distinct cell type proportions across compartments. Proportions (y axis) of cell types (x axis) in untreated (n=18), CRT (n=14), or CRTL (n=5) tumors out of all cells (top left) or in specific non-malignant cell compartments in the tumor. * p<0.05, ** p<0.01, * ** p<0.001, Mann-Whitney U test.

FIG. 26—Impact of treatment on differential gene expression in immune cells. Differential expression (0-value, x axis, mixed-effects model) and its significance (−log₁₀(adjusted p-value), y axis) for CD8+ T cells (top row), dendritic cells (second row), T_{regs} (third row) and macrophages (bottom row) (greyscale legend) in CRT vs. untreated (left), CRTL vs. untreated (middle), and CRTL vs. CRT (right) tumors. Selected induced or repressed genes are labeled. Bonferroni adjusted p-value<0.05 is indicated with a dotted horizontal line.

FIG. 27—Impact of treatment on differential gene expression in malignant cells and fibroblasts. Differential expression (0-value, x axis, mixed-effects model) and its significance (−log₁₀(adjusted p-value), y axis) for malignant cells (top row) and CAFs (bottom row) (color legend) in CRT vs. untreated (left), CRTL vs. untreated (middle), and CRTL vs. CRT (right) tumors. Selected induced or repressed genes are labeled. Bonferroni adjusted p-value<0.05 is indicated with a dotted horizontal line.

FIGS. 28A-28B—Prior signatures derived primarily from the bulk setting insufficiently delineate cells from snRNA-seq. FIG. 28A, Malignant cell signatures. UMAP embeddings of single nucleus profiles (dots) from all tumor nuclei (top panels) or only malignant cells (bottom panels) colored by expression score (greyscale bar, Methods) of signatures derived from the Bailey⁹, Collisson⁵, Moffitt⁸, and Chan-Seng-Yue⁷⁶ studies. FIG. 28B, CAF signatures. UMAP embeddings of single nucleus profiles (dots) from all fibroblast nuclei colored by normalized expression score (greyscale bar, Methods) of myCAF, apCAF, and iCAF signatures⁵⁴.

FIG. 29—Overlap between the neuronal-like program signature and genes upregulated in association with perineural invasion in PDAC. Differential expression (log₂(fold-change), x axis) and its significance (-log₁₀(adjusted p-value), y axis, DESeq2) of TCGA PDAC patients with (right) and without (left) perineural invasion. Labeled genes are present in the neuronal-like program signature.

FIGS. 30A-30B—Associations among malignant cell or CAF expression programs. Normalized correlation (color bar) among expression scores of malignant state and lineage programs across all malignant nuclei (FIG. 30A) or fibroblast programs across all fibroblast nuclei (FIG. 30B).

FIG. 31—Intra-tumoral and inter-tumoral heterogeneity of malignant and fibroblast expression programs. Normalized expression scores (y axis) of malignant state (top), malignant lineage (middle) and CAF (bottom) programs (color legend) in each untreated (n=18, left) or treated (n=25, right) tumor (x axis). Treated patients are further ordered by treatment regimen.

FIG. 32—Enrichment of malignant cell and CAF programs in genes differentially expressed with treatment regimen. Fold enrichment of overlap (x axis) between gene program signatures (top 200 genes; rows) and genes differentially expressed (q<0.05) in CRT vs. untreated (left), CRTL vs. untreated (middle), or CRTL vs. CRT (right). * Bonferroni adjusted p<0.05, hypergeometric test.

FIG. 33—. Expression of malignant lineage programs in residual neoplastic cells varies by patients' treatment response. Distribution of mean normalized expression scores in each tumor (y axis) in each pathological treatment response grade (grayscale legend) for each malignant lineage program (x axis) regardless of treatment group. * p<0.05, ** p<0.01, p<0.001, **** p<0.0001, Mann-Whitney U test.

FIG. 34—Multivariable Cox regression analysis for overall survival in TCGA and PanCuRx PDAC cohorts. Hazards ratio (middle) and p-value (left) for each variable (clinicopathologic and program expression score in bulk RNA-Seq, rows) in multivariable Cox regression model for overall survival (OS), based on a cohort of 269 patients with untreated, resected primary PDAC profiled by RNA-seq in TCGA and PanCuRx.

FIG. 35—Digital Spatial Profiling (DSP) with whole transcriptome assay (WTA). Immunofluorescence images of FFPE sections from all PDAC specimens analyzed using whole transcriptome DSP separated by treatment status (top, untreated; bottom, treated). Greyscale legend indicates target of fluorophore-conjugated antibodies.

FIG. 36—Digital spatial profiling with whole transcriptome atlas enables accurate mapping of cell type signatures in space. Expression (z-score of normalized counts across segments; purple/yellow, as represented in greyscale bar) of signature genes (rows) from different cell types (bottom greyscale legend and left bar) across segments (columns, middle greyscale legend and bottom horizontal greyscale bar) and treatment regimens (columns, top greyscale legend and top horizontal greyscale bar) profiled by WTA, capturing epithelial (green, represented in greyscale), fibroblasts (blue, represented in greyscale) and immune (red, represented in greyscale) cells. Columns and rows are clustered by unsupervised hierarchical clustering.

FIG. 37—Digital spatial profiling shows enrichment of neuronal-like and neuroendocrine-like program after neoadjuvant CRT. Distribution of Z-score normalized ssGSEA enrichment scores (y axis) of malignant (left) and fibroblast (right) programs (x axis) in AOIs from CRT (blue, represented in greyscale) and untreated (red, represented in greyscale) tumors. Box depicts interquartile range (IQR) with median marked as horizontal line. The whiskers correspond to 1.5×IQR. * p<0.05, mixed-effects model.

FIG. 38—Tumor-level feature associations based on snRNA-seq data. Unsupervised hierarchical clustering of tumor-level snRNA-seq feature correlation matrix using Pearson correlation coefficients (greyscale bar).

FIG. 39—Spatially correlated receptor-ligand pairs within compartments. Spearman rank correlation coefficient of expression of receptor-ligand pairs (gray dots) within the epithelial (left), CAF (middle) or immune (right) segments in the same ROI across all ROIs in CRT-treated (y axis) or untreated (x axis) tumors. Selected receptor-ligand pairs that were differentially correlated in CRT-treated or untreated tumors are labeled. Solid line: x=y.

The figures herein are for illustrative purposes only and are not necessarily drawn to scale.

### DETAILED DESCRIPTION OF THE EXAMPLE EMBODIMENTS

General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboraotry Manual, 2^{nd} edition 2013 (E. A Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011)

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

Pancreatic ductal adenocarcinoma (PDAC) is projected to become the second leading cause of cancer death in the United States by 2030^{1,2}. Pancreatic ductal adenocarcinoma (PDAC) remains a treatment-refractory disease. Characterizing PDAC by mRNA profiling remains particularly challenging. Previously identified bulk expression subtypes were influenced by contaminating stroma and have not yet translated into meaningful information for clinical management. Single cell RNA-seq (scRNA-seq) of fresh tumors under-represented key cell types and also thus failed to translate into clinically relevant information. More specifically, although single cell RNA-seq (scRNA-seq) can resolve these questions by distinguishing the diversity of malignant and non-malignant cells in the tumor and elucidating the impact of therapy on each compartment and their interactions, scRNA-seq in PDAC has lagged behind other cancer types due to high intrinsic nuclease content and dense desmoplastic stroma³³⁻³⁶, resulting in reduced RNA quality, low numbers of viable cells, preferential capture of certain cell types at the expense of others, and challenges with dissociating treated tumors.

Described in exemplary embodiments herein are robust single-nucleus RNA-seq (snRNA-seq) and spatial transcriptomics techniques optimized for frozen archival samples which are demonstrated using PDAC specimens. As is demonstrated in e.g., the Working Examples herein, PDAC samples from untreated and those that were from subjects that received neoadjuvant chemotherapy and radiotherapy (CRT) were analyzed using these techniques, which resulted in gene expression programs and signatures for previously unresolved subtypes and of PDAC cells. Embodiments disclosed herein provide expression signatures of PDAC tumors and methods of their use in a clinically relevant context to, among other things, improve patient treatment and prognostic stratification.

In certain example embodiments, the malignant cell signature and/or program comprises a lineage specific expression program selected from: a squamous program, a mesenchymal cytoskeletal program, mesenchymal matrisomal program, a classical progenitor program, a classical activated program, or any combination thereof; a lineage specific expression program selected from: a squamous program, a mesenchymal program, an induced basal-like program, a classical progenitor program, a classical acinar-like program, a classical neuroendocrine-like program, or any combination thereof; a cell state specific expression program selected from: a cycling program, a hypoxic program, TNF-NFkB signaling program, an interferon signaling program, or any combination thereof, a cell state specific expression program selected from: a cycling program, a TNF-NFkB signaling program, or an interferon signaling program, or any combination thereof, a neoadjuvant treated malignant cell expression program; an untreated malignant cell expression program, a cell state expression program selected from: a neuronal-like program, a neuroendocrine like program, a mesenchymal program, a squamoid program, a MYC signaling program, a cycling (G2M) program, a cycling (S) program, or any combination thereof; a lineage specific expression program selected from: an acinar-like program, a classical-like program, a basaloid program, a squamoid program, a mesenchymal program, a neuroendocrine like program, a neuronal like program, or any combination thereof, or any combination thereof.

In certain example embodiments, the CAF signature and/or program (a) comprises a myofibroblast program; a neurotropic program; a secretory program; a mesodermal progenitor program a neuromuscular program; or any combination thereof, (b) comprises a neoadjuvant treated CAF signature and/or program selected from: a neuromuscular program, a secretory program, a neurotropic program, or any combination thereof; (c) comprises an untreated CAF signature and/or program selected from: a mesodermal progenitor program, a myofibroblast program, a neurotropic program, a secretory program, or any combination thereof, or (d) comprises an adhesive expression program, an immunomodulatory expression program, a myofibroblastic progenitor expression program, or a neurotropic expression program.

In certain example embodiments, the tumor spatial neighborhood is a treatment enriched neighborhood, a squamoid-basaloid neighborhood, or a classical neighborhood.

In certain example embodiments, the one or more co-expressed receptor-ligand pairs is selected from: an Epithelial compartment—CAF compartment pair; an Epithelial compartment—Immune compartment pair; a CAF compartment and Immune compartment pair; or any combination thereof.

In certain example embodiments, wherein the malignant cell signature comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

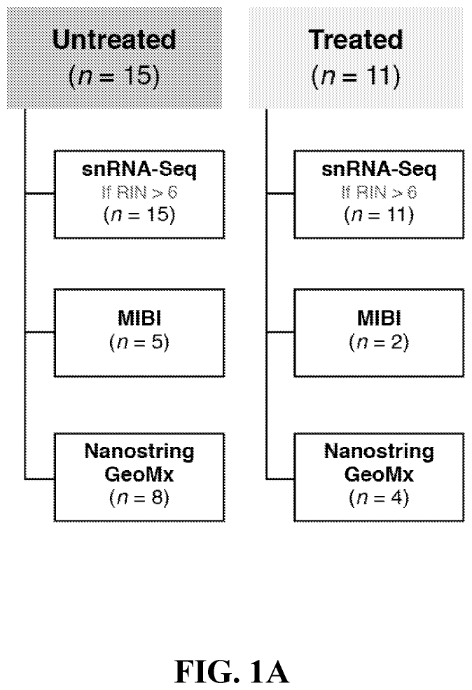

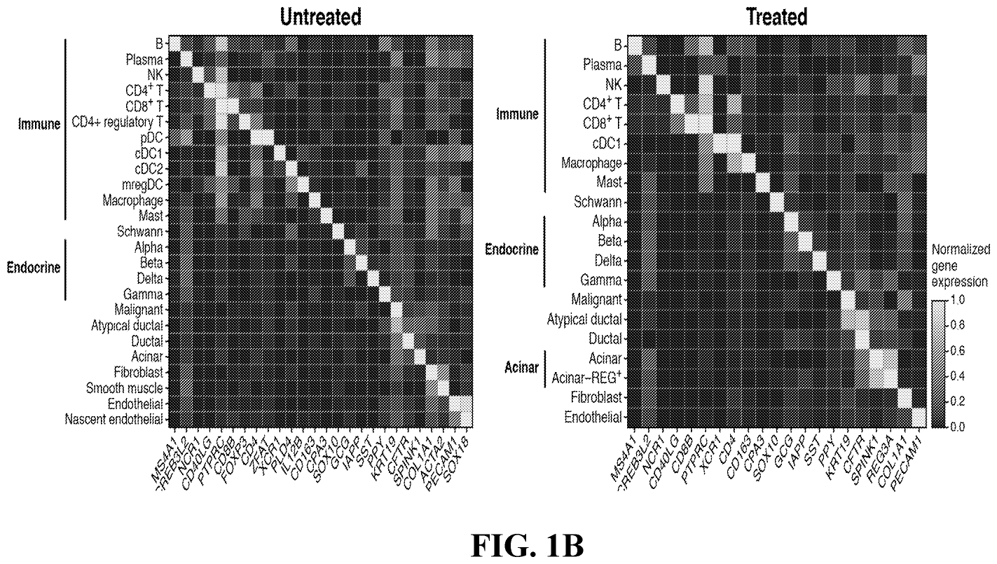

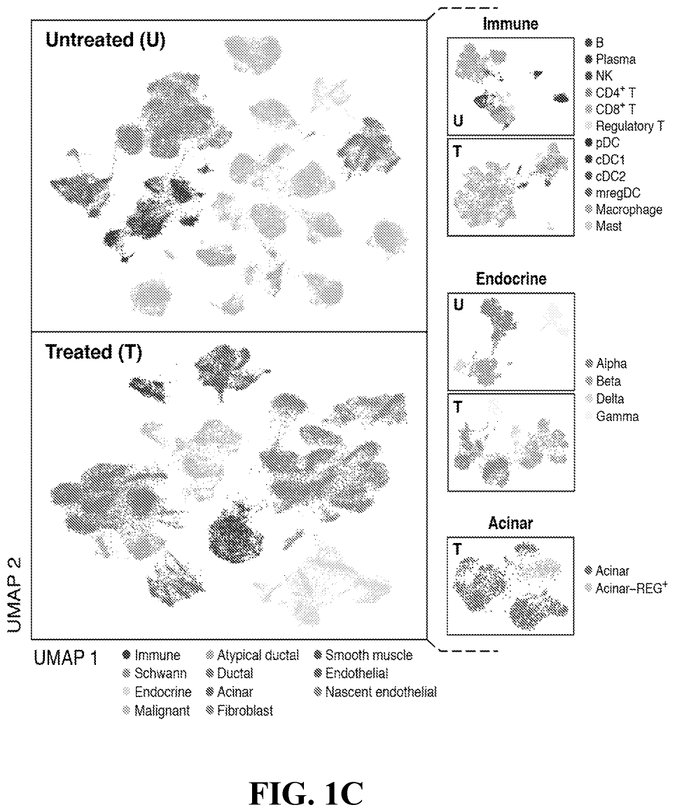

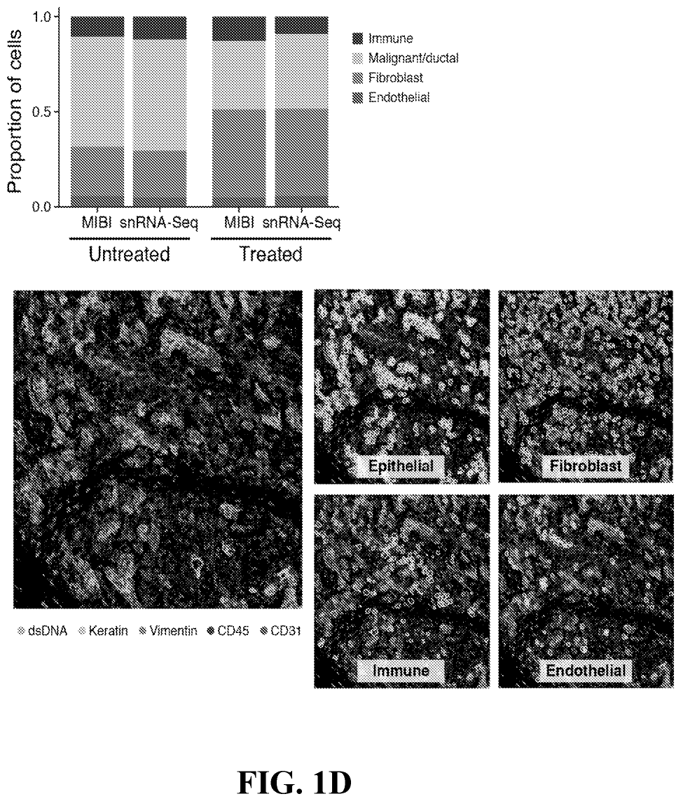

In certain example embodiments, wherein the CAF cell signature comprises one or more biomarkers, expression programs, biologic programs, receptor-ligand interactions, cell state distribution, cell type distribution, or any combination thereof as in any of FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

In certain example embodiments, the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39, and Table 7, or any combination thereof.

Described in certain example embodiments herein are methods of stratifying pancreatic ductal adenocarcinoma (PDAC) patients into treatment groups and/or prognosing PDAC or treatment outcome and/or survival in a patient comprising: detecting, in one or more a PDAC tumor cells, a malignant cell signature a cancer-associated fibroblast (CAF) signature; an immune microniche signature; or a combination thereof, wherein a characteristic regarding a patient's treatment, a patient's response to a treatment, and/or their survival is determined or predicted based on the detection of one or more of the signatures or states.

Described in certain example embodiments herein are methods of treating pancreatic ductal adenocarcinoma (PDAC) in a subject in need thereof comprising: preventing a shift in the state of a malignant cell from a classical progenitor state to a basal-like state or a terminally-differentiated state; modulating a cell state of a malignant cell from a basal-like state or a terminally-differentiated state to a classical progenitor state; inhibiting, preventing, or modulating expression of a neuronal like expression program in a malignant cells; inhibiting, preventing expression or modulating expression of a malignant squamoid expression program in a malignant cell, inhibiting, preventing, or modulating expression of an adhesive CAF expression program in a CAF cell; or any combination thereof.

Described in certain example embodiments herein are methods of screening for one or more agents capable of modulating a PDAC malignant cell state comprising: contacting a cell population comprising PDAC malignant cells having an initial cell state with a test modulating agent or library of modulating agents; determining a fraction of malignant cells having a desired cell state and an undesired cell state; selecting modulating agents that shift the initial PDAC malignant cell state to a desired cell state or prevent the initial PDAC malignant cell state to shift from a desired initial state, such that the fraction of PDAC malignant cells in the cell population having a desired cell state is above a set cutoff limit.

Described in certain example embodiments herein are methods method of treating a subject having pancreatic ductal adenocarcinoma (PDAC), the method comprising: administering a neoadjuvant therapy to the subject; and administering a PDAC malignant cell modulating agent to the subject, administering an immune modulator to the subject, administering a CAF modulating agent to the subject, or any combination thereof to the subject.

Described in certain example embodiments herein are methods method of treating a subject having PDAC, the method comprising: detecting, in one or more PDAC tumor cells, a malignant cell signature; a cancer-associated fibroblast (CAF) signature; an immune microniche signature; or a combination thereof; and administering a PDAC treatment to the subject in need thereof.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Expression Signatures and Programs

Described herein are PDAC tumor signatures and/or programs including, but not limited to, a malignant signature and/or program, a CAF signature and/or program, an immune microniche signature and/or program, a tumor spatial neighborhood; one or more co-expressed receptor-ligand pairsor a combination thereof. In some embodiments the PDAC tumor signatures and/or programs include a neoadjuvant treated tumor expression program (“a treated program”); or a neoadjuvant untreated tumor expression program (an “untreated program”). In some embodiments, the PDAC tumor signature and/or program is a malignant cell signature and/or program. In some embodiments, the PDAC tumor signature and/or program is a CAF signature and/or program. In some embodiments, the PDAC tumor signature and/or program is an immune microniche signature and/or program,

In certain example embodiments, the malignant cell signature and/or program comprises a lineage specific expression program selected from: a squamous program, a mesenchymal cytoskeletal program, mesenchymal matrisomal program, a classical progenitor program, a classical activated program, or any combination thereof; a lineage specific expression program selected from: a squamous program, a mesenchymal program, an induced basal-like program, a classical progenitor program, a classical acinar-like program, a classical neuroendocrine-like program, or any combination thereof; a cell state specific expression program selected from: a cycling program, a hypoxic program, TNF-NFkB signaling program, an interferon signaling program, or any combination thereof, a cell state specific expression program selected from: a cycling program, a TNF-NFkB signaling program, or an interferon signaling program, or any combination thereof, a neoadjuvant treated malignant cell expression program; an untreated malignant cell expression program, a cell state expression program selected from: a neuronal-like program, a neuroendocrine like program, a mesenchymal program, a squamoid program, a MYC signaling program, a cycling (G2M) program, a cycling (S) program, or any combination thereof; a lineage specific expression program selected from: an acinar-like program, a classical-like program, a basaloid program, a squamoid program, a mesenchymal program, a neuroendocrine like program, a neuronal like program, or any combination thereof, or any combination thereof.

In certain example embodiments, the CAF signature and/or program (a) comprises a myofibroblast program; a neurotropic program; a secretory program; a mesodermal progenitor program a neuromuscular program; or any combination thereof, (b) comprises a neoadjuvant treated CAF signature and/or program selected from: a neuromuscular program, a secretory program, a neurotropic program, or any combination thereof; (c) comprises an untreated CAF signature and/or program selected from: a mesodermal progenitor program, a myofibroblast program, a neurotropic program, a secretory program, or any combination thereof, or (d) comprises an adhesive expression program, an immunomodulatory expression program, a myofibroblastic progenitor expression program, or a neurotropic expression program.

In certain example embodiments, the tumor spatial neighborhood is a treatment enriched neighborhood, a squamoid-basaloid neighborhood, or a classical neighborhood.

In certain example embodiments, the one or more co-expressed receptor-ligand pairs is selected from: an Epithelial compartment—CAF compartment pair; an Epithelial compartment—Immune compartment

In certain example embodiments, the malignant cell signature comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

In certain example embodiments, the CAF cell signature comprises one or more biomarkers, expression programs, biologic programs, receptor-ligand interactions, cell state distribution, cell type distribution, or any combination thereof as in any of FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

In certain example embodiments, the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39 and Table 7, or any combination thereof.

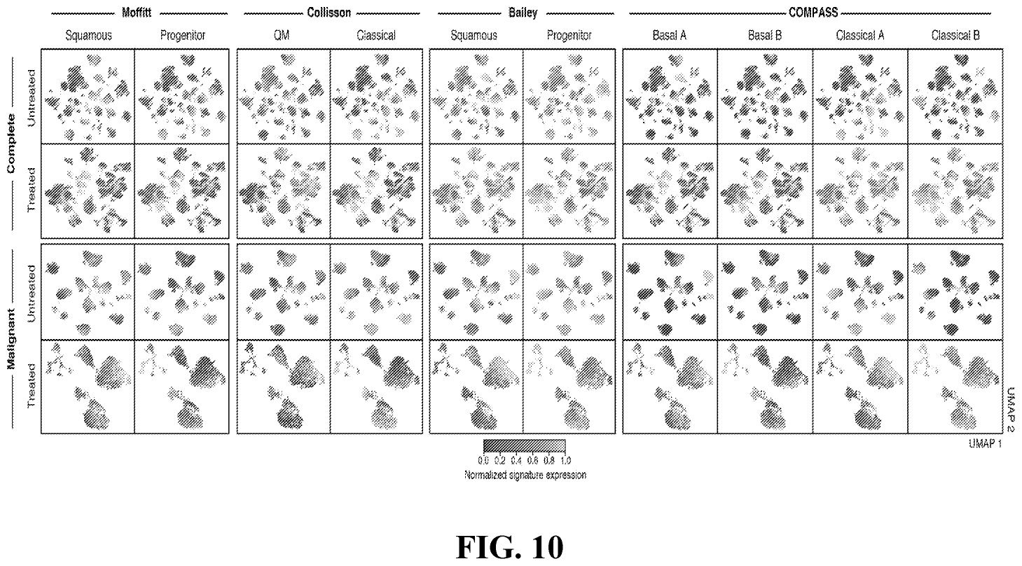

In certain example embodiments, a treated PDAC tumor signature and/or program comprises one or more biomarkers, expression programs, biologic programs, receptor-ligand interactions, cell state distribution, cell type distribution, or any combination thereof as in any of FIGS. 1B-1D, 2A-2D, 3A-3C, 4A-4C, 5A-5C, 7, 9A-9D, 10, 11, 12, 14, 15A-15D, 17B-17G, 18A-18D, 19A-19D, 20A-20C, 21A-21B, 23, 24, 25, 26, 26, 29, 30, 31, 32, 36, 37, 38, 39, and Tables 2.1-2.6, and any combination thereof.

In certain example embodiments, an untreated PDAC tumor signature and/program one or more biomarkers, expression programs, biologic programs, receptor-ligand interactions, cell state distribution, cell type distribution, or any combination thereof as in any of FIGS. 1B-1D, 2A-2D, 3A-3C, 4A-4C, 5A-5C, 7, 9A-9D, 10, 11, 12, 14, 15A-15D, 17B-17G, 18A-18D, 19A-19D, 20A-20C, 21A-21B, 23, 24, 25, 26, 26, 29, 30, 31, 32, 36, 37, 38, 39, and Tables 2.1-2.6, and any combination thereof.

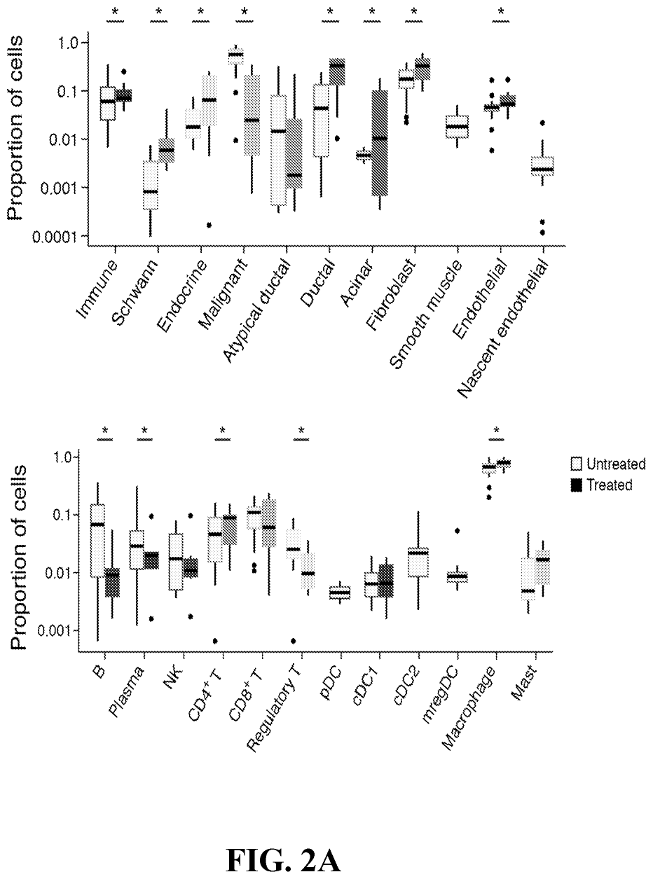

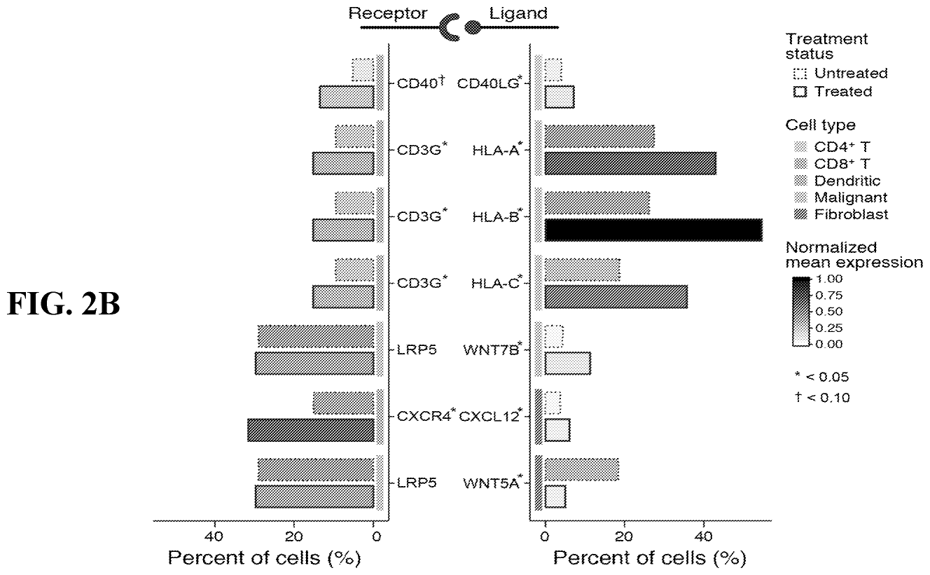

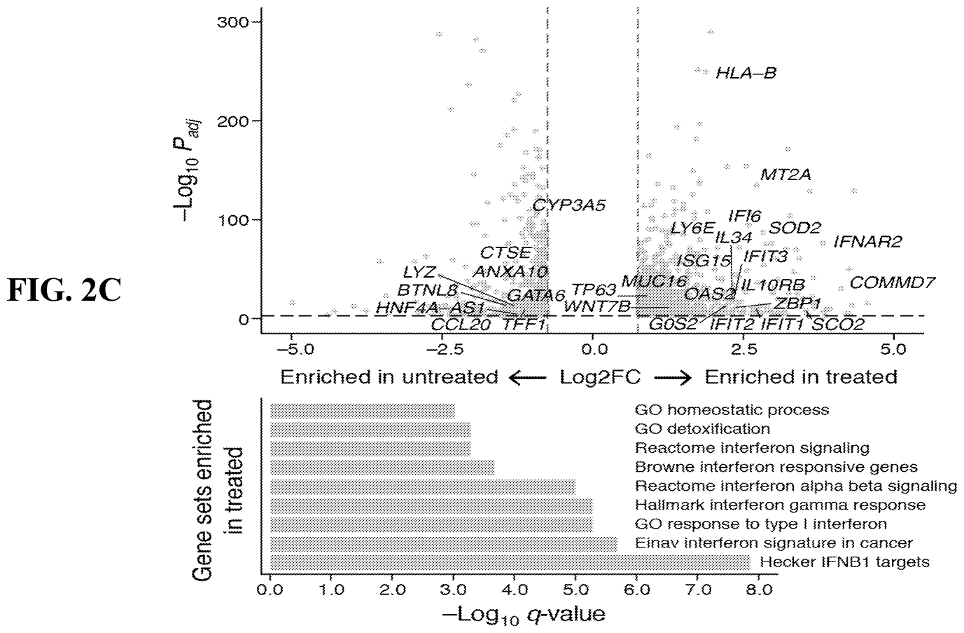

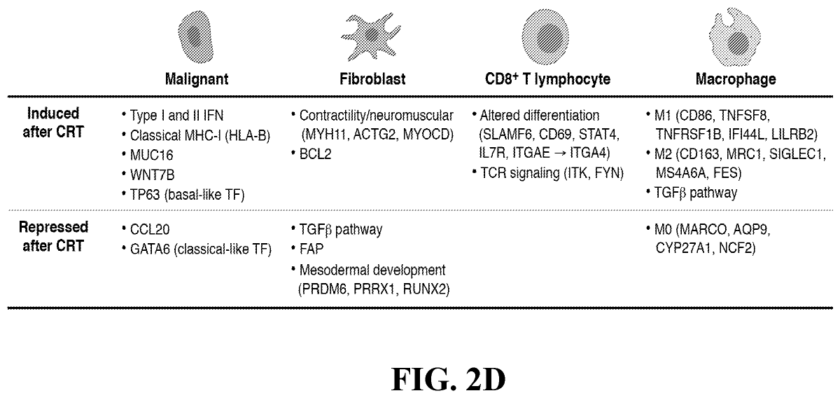

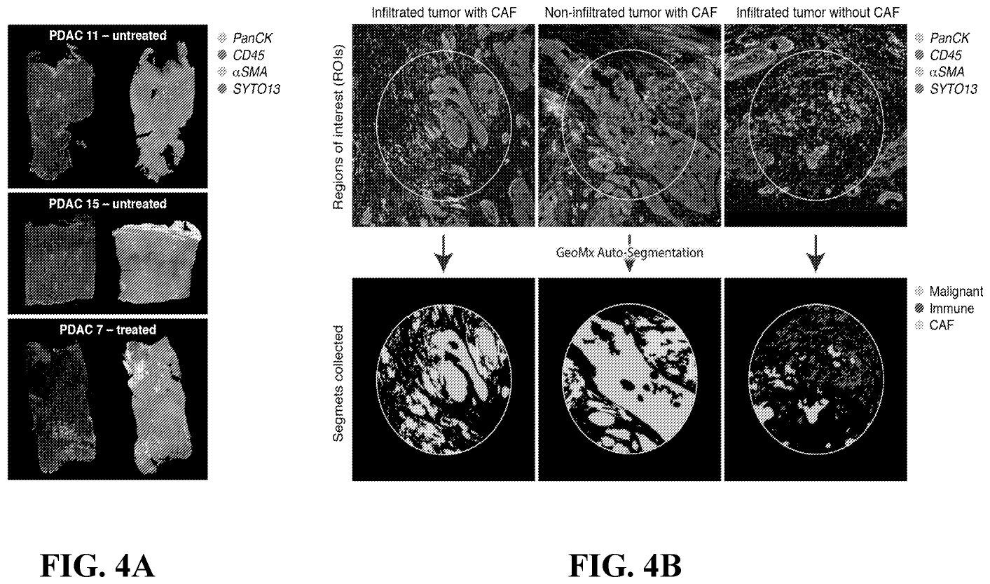

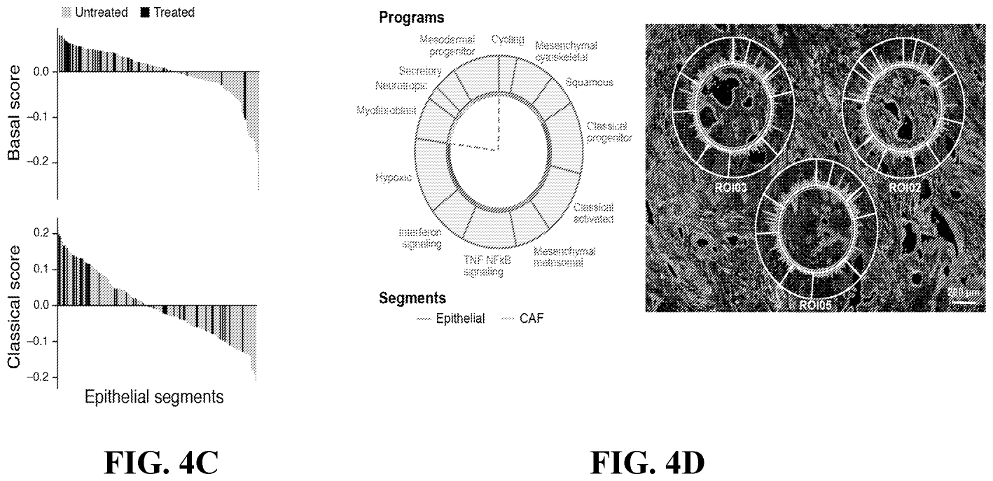

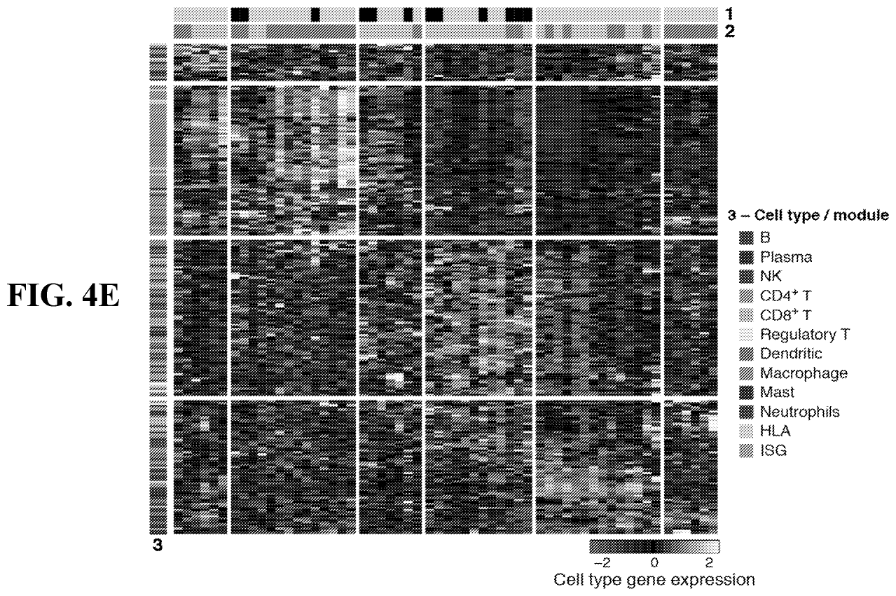

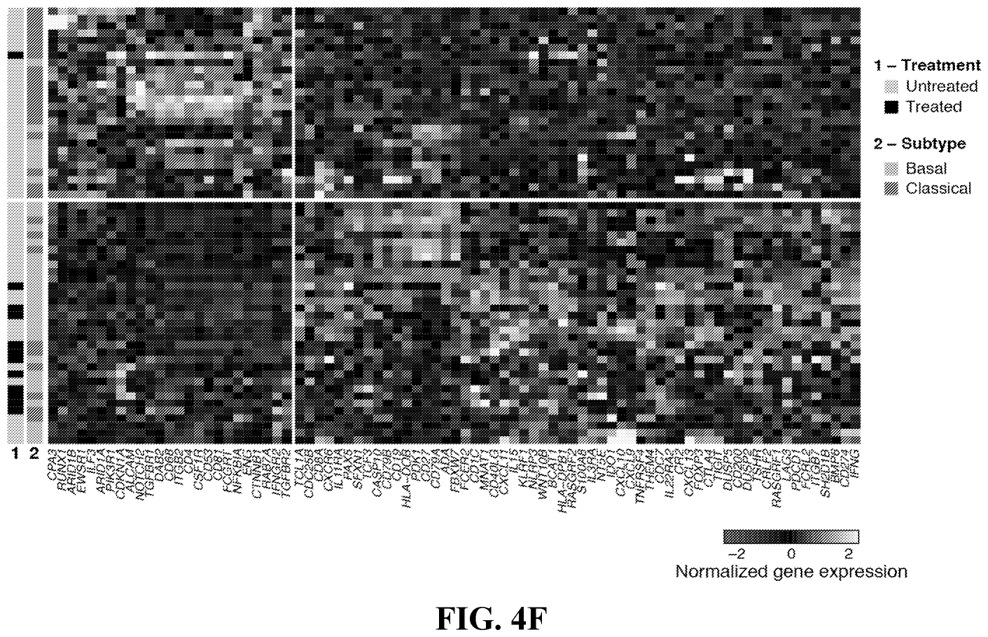

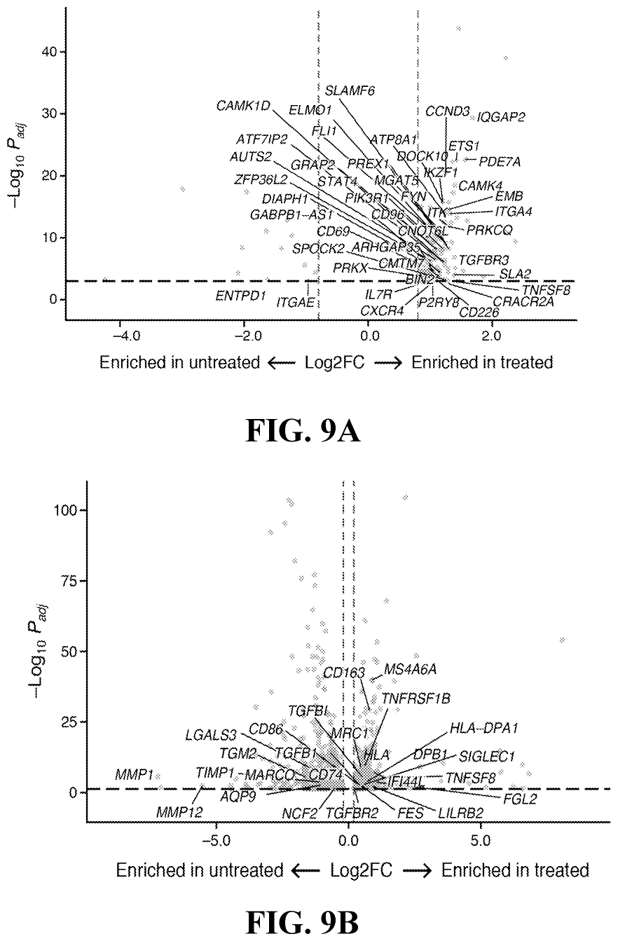

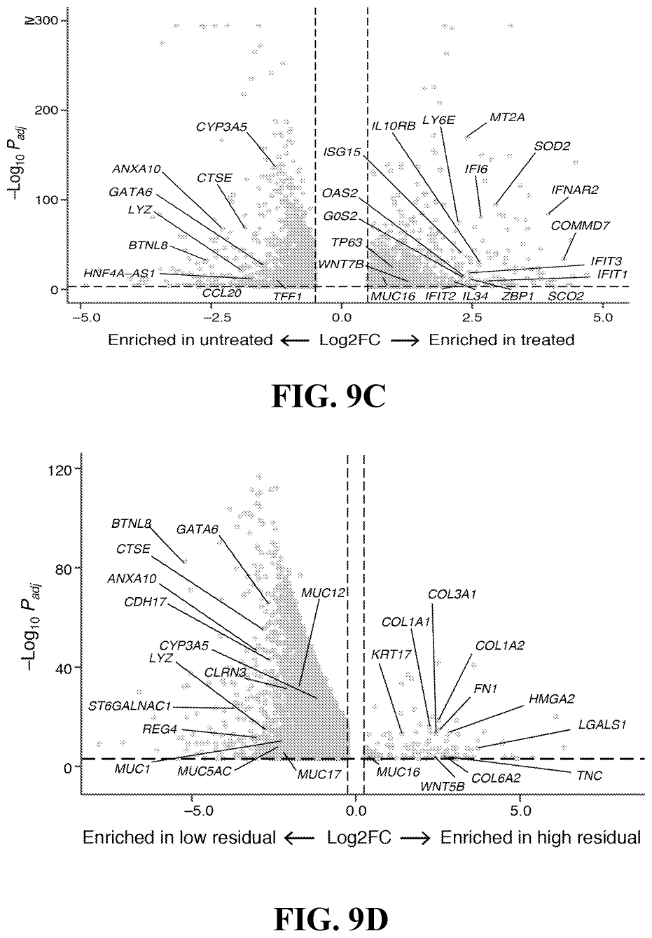

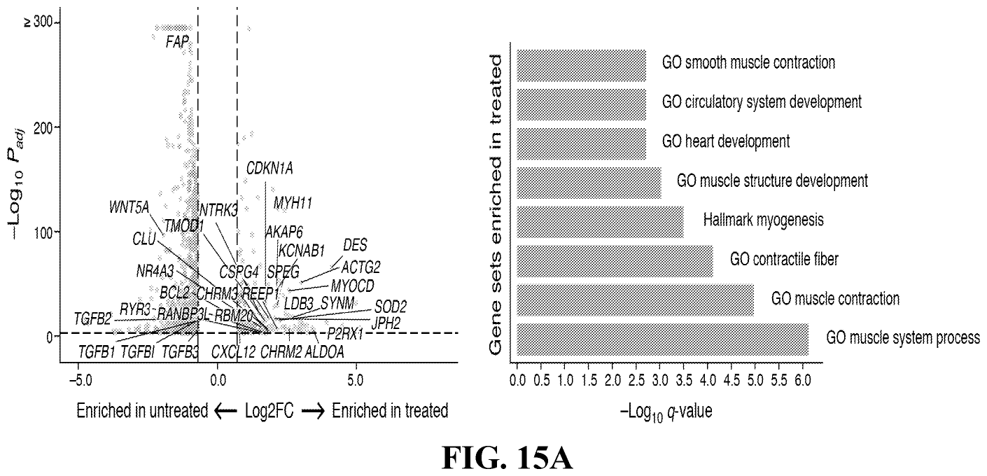

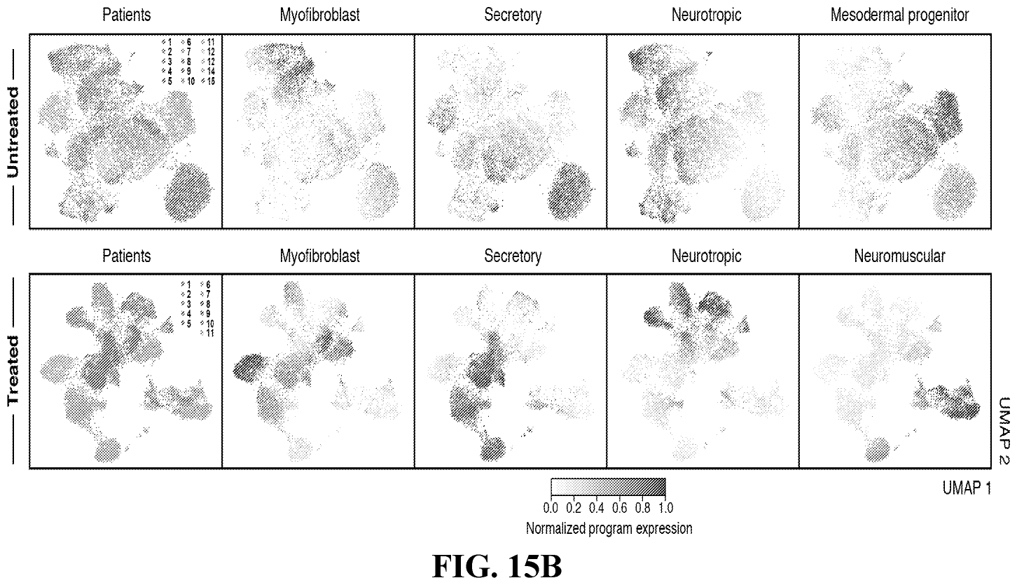

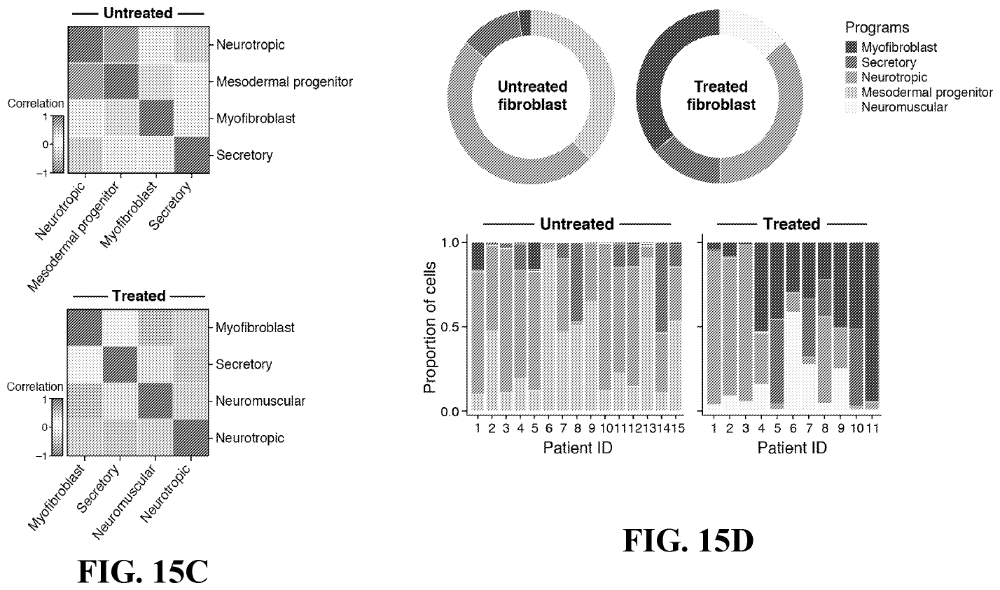

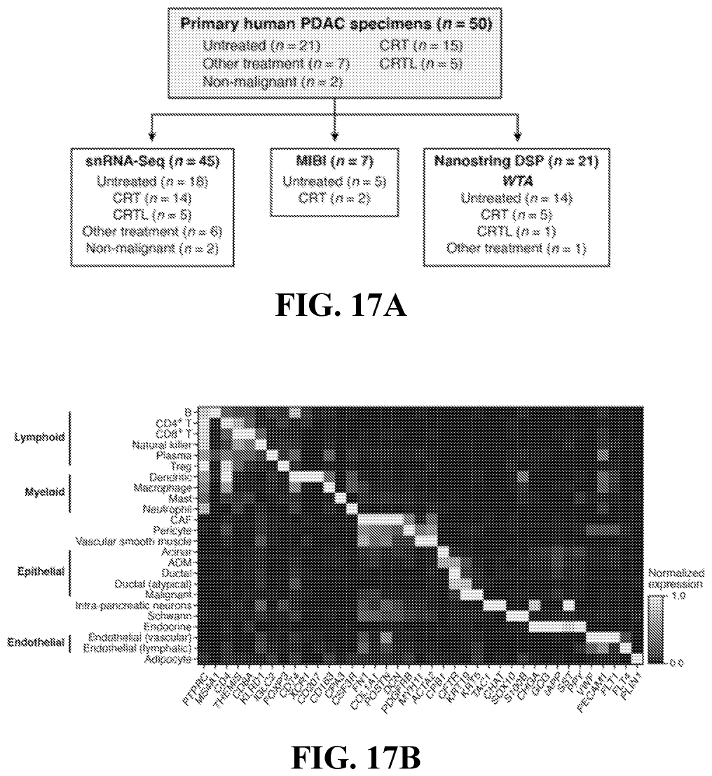

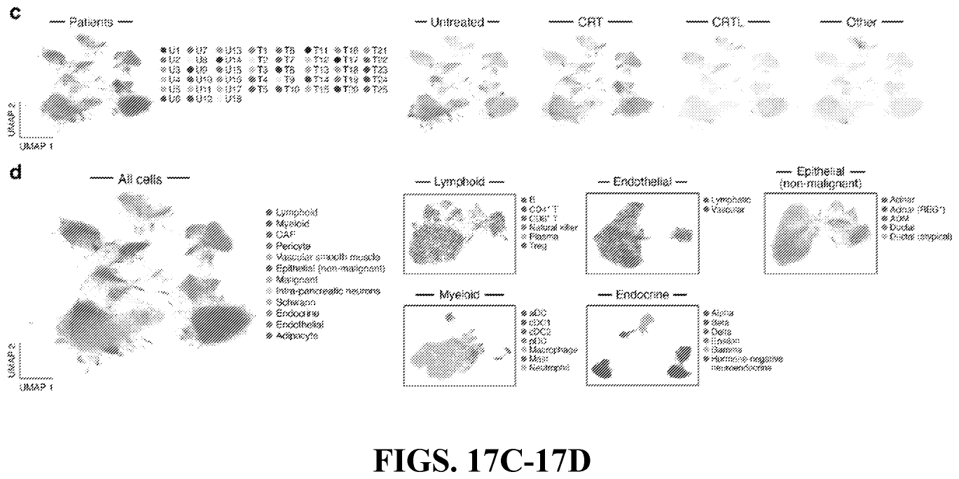

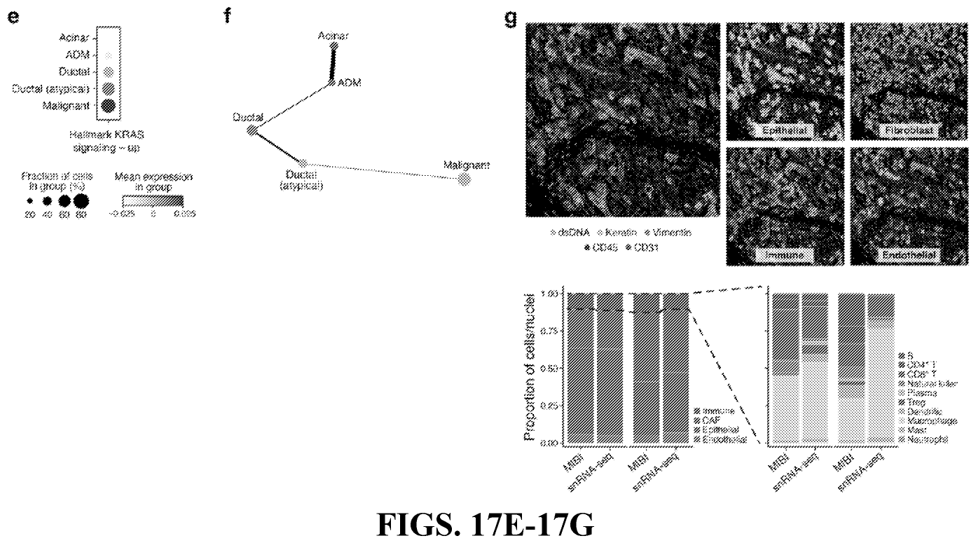

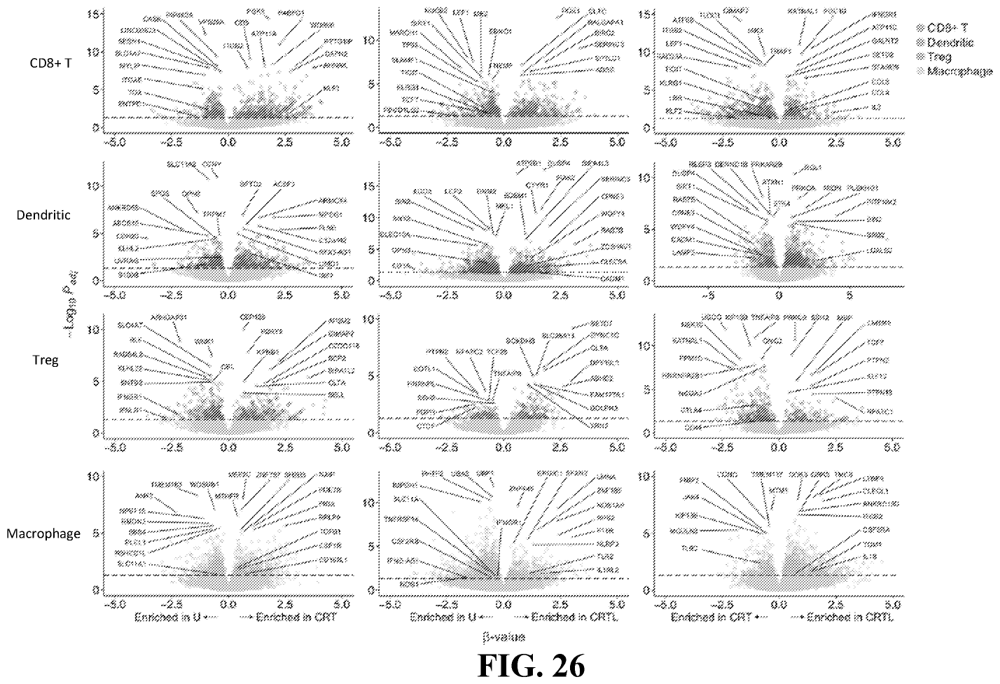

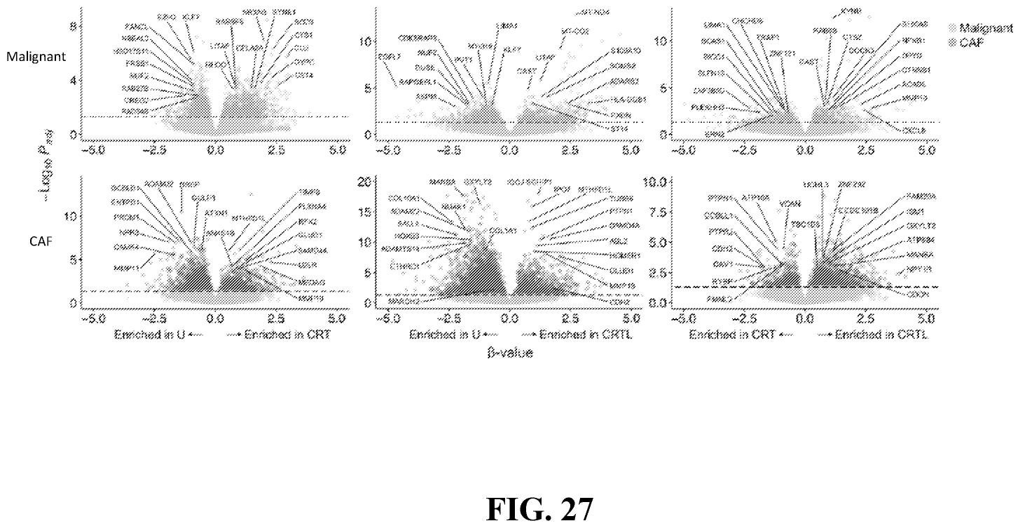

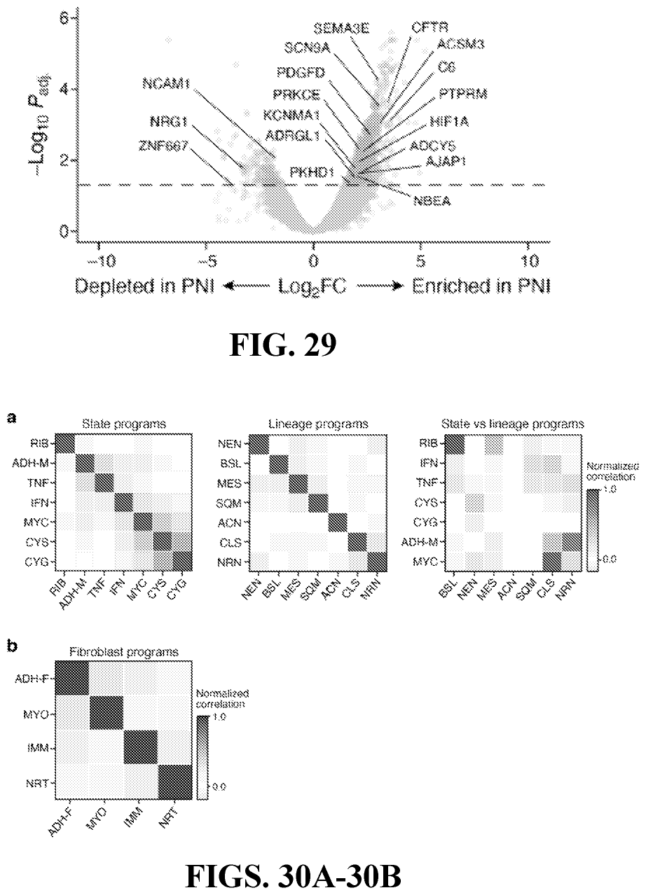

In certain example embodiments, the CAF signature and/or program comprises one or more of the following genes or groups of genes: keratin, vimentin, CD45, CD31, or any combination thereof (see e.g., FIG. 1D; MSA4A1, CREB3L2, NCR1, CD40LG, PTPRC, CD8B, FOXP3, CD4, ZFAT, XCR1, PLD4, IL12B, CD163, CPA3, SOX10, GCG, IAPP, SST, PPY, KRT19, CFTR, SPINK1, COL1A1, ACTA2, PECAM, SOX18, or any combination thereof (see e.g., FIG. 2A); CD40, CD3G, LRP5, CXCR4, or any combination thereof (see e.g., FIG. 2B); CD40LG, HLA-A, HLA-B, HLA-C, WNT7B, CXCL12, WNT5A, or any combination thereof (see e.g., FIG. 2B); CYP3A5, CTSE, ANXA10, LYZ, BTNL8, GATA6, HNF4A, AS1, CCL20, TFF1, HLA-B, MT2A, IF16, LY6E, SOD2, IL34, IFNAR2, ISG15, IFIT3, MUC16, OAS2, IL1ORB, COMMD7, ZBP1, TP63, WNT7B, GOS2, IFIT2, IFIT1, SCO2, and any combination thereof (see e.g., FIG. 2C); CYP3A5, CTSE, ANXA10, LYZ, BTNL8, GATA6, HNF4A, AS1, CCL20, TFF1, or any combination thereof (see e.g., FIG. 2C),; HLA-B, MT2A, IF16, LY6E, SOD2, IL34, IFNAR2, ISG15, IFIT3, MUC16, OAS2, IL1ORB, COMMD7, ZBP1, TP63, WNT7B, GOS2, IFIT2, IFIT1, SCO2, or any combination thereof, (see e.g., FIG. 2C); Type I IFN, Type II IFN, or both (see e.g., FIG. 2D); MHC-I (HLAB), MUC16, WNT7B, TP63, or any combination thereof (see e.g., FIG. 2D; CCL20, GATA6, or both (see e.g., FIG. 2D); MYH11, ACTG2, MYOCD, BCL2, or any combination thereof (see e.g., FIG. 2D); a TGFbeta pathway gene, FAP, PRDM6, ORRX1, RUNX2 or any combination thereof (see e.g., FIG. 2D); SLAMF6, CD69, STAT4, IL7R, ITGAE, ITGA4, or any combination thereof (see e.g., FIG. 2D); ITK, FYN, or both, (see e.g., FIG. 2D); CD86, TNFSF8, TNFRSFIB, IFI44L, LILRB, or any combination thereof (see e.g., FIG. 2D); CD163, MRC1, SIGLEC1, MS4A6A, FES, or any combination thereof(see e.g., FIG. 2D); MARCO, AQP9, CYP27A1, NCF2, or any combination thereof (see e.g., FIG. 2D); PanCK, CD45, alphaSMA, SYTO13, or any combination thereof (see e.g., FIGS. 4A-4B); CPA3, RUNX1, ARIDIB, EWSR1, ILF3, ARID1A, PIK3R1, CDKN1A, ALCAM, NOTCH2, TGFBR1, DAB2, CD68, ITGB2, CD4, CSF1R, CD53, CD81, FCGRT, NFKBIA, ENG, CTNNB1, RAB7A, IFNGR2, TGFBR2, TCL1A, CDC25B, CD8A, CCXCR6, IL11RA, PAX5, SFXN1, TCF3, CASP10, CD79B, CD19, HLA-DOB, PDK1, CD27, CD79A, ADA, FBXW7, FCER2, CD1C, MNAT1, CD40LG, CXCL11, IL15, KLRF1, NLRP3, WNT10B, BCAT1, HLA-DRB1, RASGRF2, S100A8, IL3RA, NT5E, IDO1, CXCL10, CXCL9, TNFRSF4, THEM4, CDC7, IL22RA2, CR2, CXCL13, FOXP3, CTLA4, TIGIT, DUSP5, CD200, DUSP2, TSHR, CRLF2, RASGRF1, LAG3, PDCD1, FCRL2, ITGB7, SH2D1B, BMP6, CD274, IFNG, or any combination thereof (see e.g., FIG. 4F); ENTPD1, ITGAE, GABPB1, AS1, or any combination thereof (see e.g., FIG. 9A); CAMK1D, ELMO1, SLAMF6, CCND3, IQGAP2, ATF7IP2, FL11, ATP8A1, ETS1, AUTS2, GRAP2, PREK1, DOCK10, PDE7A, ZFP36L2, STAT4, MGAT5, IKZF1, CAMK4, DIAPH1, PIK3R1, FYN, EMB, CD96, ITK, ITGA4, CD69, CNOT6L, PRKCQ, SPOCK2, ARHGAP35, PRKX, CMTM7, TGFBR3, BIN2, IL7R, SLA2, CXCR4, P2RY8, CD226, CRACR2A, TNFSF8, or any combination thereof (see e.g., FIG. 9A),; ENTPD1, ITGAE, GABPB1, AS1, CAMK1D, ELMO1, SLAMF6, CCND3, IQGAP2, ATF7IP2, FL11, ATP8A1, ETS1, AUTS2, GRAP2, PREK1, DOCK10, PDE7A, ZFP36L2, STAT4, MGAT5, IKZF1, CAMK4, DIAPH1, PIK3R1, FYN, EMB, CD96, ITK, ITGA4, CD69, CNOT6L, PRKCQ, SPOCK2, ARHGAP35, PRKX, CMTM7, TGFBR3, BIN2, IL7R, SLA2, CXCR4, P2RY8, CD226, CRACR2A, TNFSF8, or any combination thereof, (see e.g., FIG. 9A); MMP1, MMP12, AQP9, NCF2, TIMP1, MARCO, CD74, TGM2, TGFB1, LGALS3, or any combination thereof (see e.g., FIG. 9B); CD86, TGFB1, MRC1, TGFBR2, FES, CD163, HLA, IFI44L, FES, LILRBB2, FGL2, TNFSF8, DBPB1, SIGLEC1, HLA-DPA1, MS4A6A (see e.g., FIG. 9B); MMP1, MMP12, AQP9, NCF2, TIMP1, MARCO, CD74, TGM2, TGFB1, LGALS3, CD86, TGFB1, MRC1, TGFBR2, FES, CD163, HLA, IFI44L, FES, LILRBB2, FGL2, TNFSF8, DBPB1, SIGLEC1, HLA-DPA1, MS4A6A (see e.g., FIG. 9B); TFF1, CCL20, HNF4A, AS1, BTNL8, LYZ, GATA6, ANXA10, CTSE, CYP3A5, or any combination thereof (see e.g., FIG. 9C); ISG15, OAS2, GOS2, TP63, WNT7B, IL1ORB, LY6E, IFI6, MT2A, SOD2, IFNAR2, COMMD7, IFIT3, IFIT1, SCO2, ZBP1, IL34, IFIT2, MUC16, or any combination thereof (see e.g., FIG. 9C); TFF1, CCL20, HNF4A, AS1, BTNL8, LYZ, GATA6, ANXA10, CTSE, CYP3A5, ISG15, OAS2, GOS2, TP63, WNT7B, IL1ORB, LY6E, IFI6, MT2A, SOD2, IFNAR2, COMMD7, IFIT3, IFIT1, SCO2, ZBP1, IL34, IFIT2, MUC16, or any combination thereof (see e.g., FIG. 9C); BTNL8, GATA6, CTSE, ANXA10, CDH17, MUC12, CYP3A5, LYZ, CLRN3, ST6GALNAC1, REG4, MUC1, MUC5AC, MUC17, or any combination thereof (see e.g., FIG. 9D; COL1A1, COL3A1, KRT17, COL1A2, FN1, HMGA2, LGALS1, MUC116, WNT5B, COL6A, TNC, or any combination thereof (see e.g., FIG. 9D); BTNL8, GATA6, CTSE, ANXA10, CDH17, MUC12, CYP3A5, LYZ, CLRN3, ST6GALNAC1, REG4, MUC1, MUC5AC, MUC17, COL1A1, COL3A1, KRT17, COL1A2, FN1, HMGA2, LGALS1, MUC116, WNT5B, COL6A, TNC, or any combination thereof (see e.g., FIG. 9D); TGFB3, TGFBI, TGFB1, TGFB2, FAP, WNT5A, or any combination thereof (see e.g., FIG. 15A); CDKN1A, NTRK3, MYH11, TMOD1, CLU, NR4A3, BCL2, RYR3, RANBP3L, CSPG4, CHRM3, RBM20, CXCL12, REEP1, CHRM2, ALDOA, P2RX1, LDB3, SPEG, KCNAB1, AKAP6, DES, ACTG2, MYOCD, SYNM, SOD2, JPH2, or any combination thereof (see e.g., FIG. 15A); TGFB3, TGFBI, TGFB1, TGFB2, FAP, WNT5A, CDKN1A, NTRK3, MYH11, TMOD1, CLU, NR4A3, BCL2, RYR3, RANBP3L, CSPG4, CHRM3, RBM20, CXCL12, REEP1, CHRM2, ALDOA, P2RX1, LDB3, SPEG, KCNAB1, AKAP6, DES, ACTG2, MYOCD, SYNM, SOD2, JPH2, or any combination thereof (see e.g., FIG. 15A); PTPRC, MS4A1, CD4, THEMIS, CD8A, KLDR1, IGLC2, FOXP3, CD74, XCR1, CD207, CD163, CPA3, CSF3R, FN1, COL1A1, POSTN, DCN, PDGFRB, MYH11, ACTA2, CPB1, CFTR, KRT19, KRT5, TAC1, CHAT, SOX10, S100B, CHGA, IAPP, SST, PPY, VWF, PECAM1, FLT1, FLT4, PLIN1, or any combination thereof (see e.g., FIG. 17B); oENTPD1, TOX, ITGAE, MYLIP, SLC4A7, SESN1, LINC00623, CASK, PIP4K2A, VPS26A, ITGB2 CD5, PGK1, ATP11A, PABPC1, WDR60, PTTG1IP, CAPN2, AHNAK, KLF2, or any combination thereof(see e.g., FIG. 26); S100B, UVRAG, KLHL2, CDH20, ABCB10, ANKRD55, EPG5, OPN2, TRPM7, SLC11A2, CCNY, SFTD2, ACSF3, ARMCX4, MPEG1, FLNB, C12orf42, RFX3-AS1, LIMD1, IRF7, or any combination thereof (see e.g., FIG. 26); IFNLR1, IFNGR1, SNTB2, KLHL22, RAD54L2, RLF, SLC4A7, ARHGAP31, WNK1, CBL, CEP128, P2RY8, KPNB1, AP3M2, GIMAP7, CCDC116, SCP2, SIPA1L2, CLTA, SELL, or any combination thereof (see e.g., FIG. 26); SLC11A1, R3HCC1L, PLCL1, BBS4, RMDN3, NPBF15, ANK2, TMEM163, WDSUB1, MTHFR, MEF2C, ZNF787, SH2B3, NAIP, PDE78, PIGX, RPLP0, TGFB1, CSF1R, CD163L1, or any combination thereof (see e.g., FIG. 26); PDCD1LG2, TCF7, KLRG1, TIGIT, SLAMF1, TP53, MARCH1, SIRT1, NUCB2, LEF1, ME2, SBNO1, NEMF, PGK1, CLTC, RALGAPA1, BIRC2, SERINC3, SPTLC1, ADSS, or any combination thereof (see e.g., FIG. 26); CD1A, OPN3, CLEC10A, SNX2, BIN2, AGO4, LCP2, DNM2, MKL1, EDEM1, CYYR1, ATP2B1, DUSP4, IRAK2, SIPA1L3, SERINC3, CPNE3, WDFY4, RAB7B, ZC3HAV1, CLEC9A, CADM1, or any combination thereof (see e.g., FIG. 26); CTCF, PDPR, HNRNPL, COTL1, PTPN2, NFATC2, TCF25, TNFAIP8, BCKDHB, SLC25A13, SETD7, DYNC1L2, CLTA, DPY19L1, ABHD2, FAM177A1, GOLPH3, XRN2, or any combination thereof (see e.g., FIG. 26); NOS1, IFNG-AS1, CSF2RB, TNFRSF14, SLC11A, IMPDH1, PHTF2, UBA3, UBP1, IFNGR1, ZNF445, ERGIC1, SFXN2, LMNA, ZNF185, NOS1AP, RPS2, F11R, NLRP3, TLR2, IL1RL2, or any combination thereof (see e.g., FIG. 26); KLF2, LBR, KLRG1, TIGIT, RAD23A, LEF1, ITGB2, ATF6B, TLDC1, GIMAP7, JAK3, TRAF1, KATNAL1, POC1B, IFNGR1, ATP1 IC, GALNT2, SETD2, STARD9, CCL5, CCL4, IL2, or any combination thereof (see e.g., FIG. 26); LAMP3, CADM1, WDFY4, CPNE3, RAB7B, SIRT1, DUSP4, REEP3, DENND1B, PRKAR2B, ATXN1, ATK4, RGL1, PRKCA, MIDN, PLEKHO1, PITPNM2, BIN2, BRD2, LGALS2, or any combination thereof (see e.g., FIG. 26); CD44, CTLA4, NCOA3, HNRPA2B1, PPM1D, KATNAL1, NEK10, UGCG, KIF13B, TNFAIP3, CNG2, PRKCB, SSH2, MBP, CMTM8, TCF7, PTPN2, KLF12, PTPN13, NFATC1, or any combination thereof (see e.g., FIG. 26); TLR2, MCOLN3, KIF13B, JAK4, FNIP2, CCNC, TMEM117, MTM1, DOK3, GRK5, TMC8, LTBP4, CLECL1, ANKRD13D, ITGB2, CSF2RA, TGM1, IL1B, or any combination thereof (see e.g., FIG. 26); RAD54B, CREG2, RAB27B, NUF2, FRSS1, HSD17B11, NBEAL2, FANCL, EZH2, KLF7, RHOD, LITAF, RASSF5, CELA2A, NRXN3, SYNE1, SOD3, CYS1, CLU, GYPC, CST4 or any combination thereof (see e.g., FIG. 27); MMP11, CAMK4, NPR3, PRDM1, ENTPD1, DCBLD1, ADAM22, NREP, GULP1, ATXN1, ANKS1B, MTHFD1L, TIMP3, PLXNA4, RFX2, GLUD1, SAMD4A, LDLR, MEDAG, MMPP19, or any combination thereof (see e.g., FIG. 27); EGFL7, ASPM, RAPGEFL1, DUSIL, NUF2, CDK5RAP3, PVT1, MYO198, LIMA1, KLF7, CAST, LITAF, MT-C02, MT-ND4, S100A10, SGMS2, ADARB2, HLA-DQB1, PXDN, ST14, or any combination thereof (see e.g., FIG. 27); ARCH2, CTHRC1, ADAMTS14, HOXB3, SALL4, ADAM22, COL10A1, NUAK1, MANBA, GXYLT2, COL1A1, IQCJ-SCHIP1, IPO7, MTHFD1L, TUBB6, PTPN1, SAMD4A, ABL2, HOMER1, GLUD1, MMP19, CDH2, or any combination thereof (see e.g., FIG. 27); ERN2, PLEKHH3, ZNF385D, SLFN13, BICC1, BCAS1, LIMA1, CHCD6, TRAP1, ZNF121, CAST, RAB88, KYNU, CTSZ, DOCK3, S100AB, NFKB1, DPYD, CTNNB1, ACADL, MMP10, CXCL8, or any combination thereof (see e.g., FIG. 27); FMNL2, RYBP, CAV1, CDH2, PTPRJ, COBLL1, PTPN1, ATP10A, VCAN, TBC1D5, UCHL3, ZNF292, CCDCl01B, FAM20A, ISM1, GXYLT2, ATP8B4, MANBA, NPY1R, CDON, or any combination thereof (see e.g., FIG. 27), ZNF667, NRG1, NCAM1, PKHD1, ADRGL1, KCNMA1, PRKCE, PDGFD, SCN9A, SEMA3E, CTFR, ACSM3, C6, PTPRM, HIF1A, ADCY5, AJAP1, NBEA, or any combination thereof (see e.g., FIG. 29); or any combination thereof.

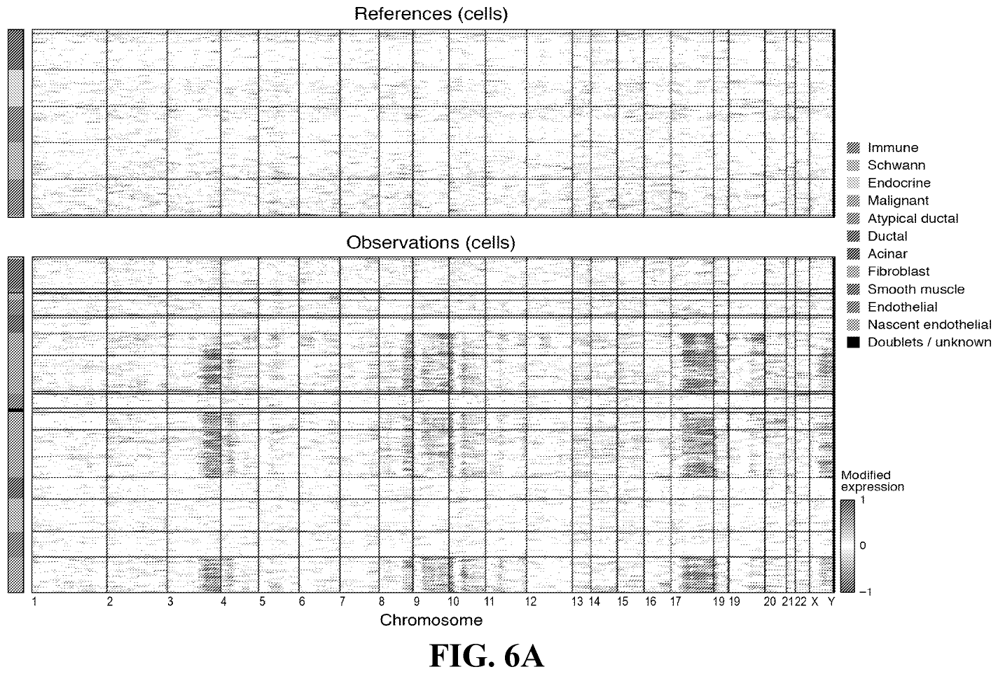

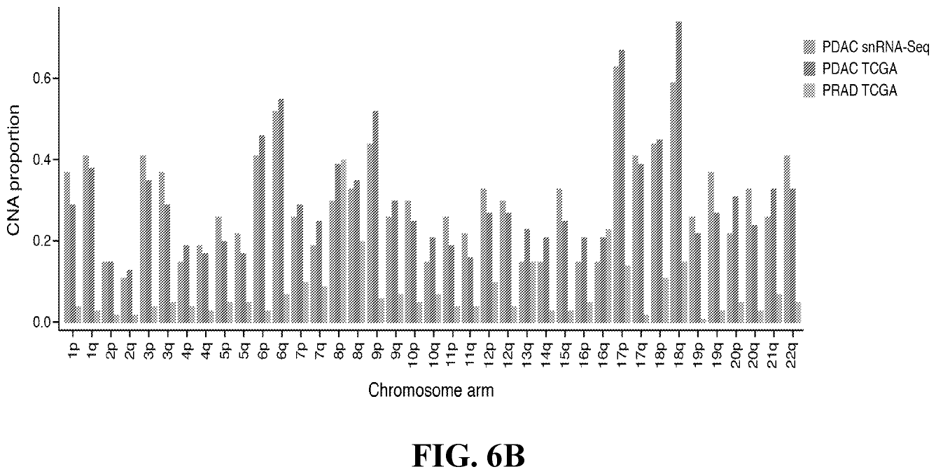

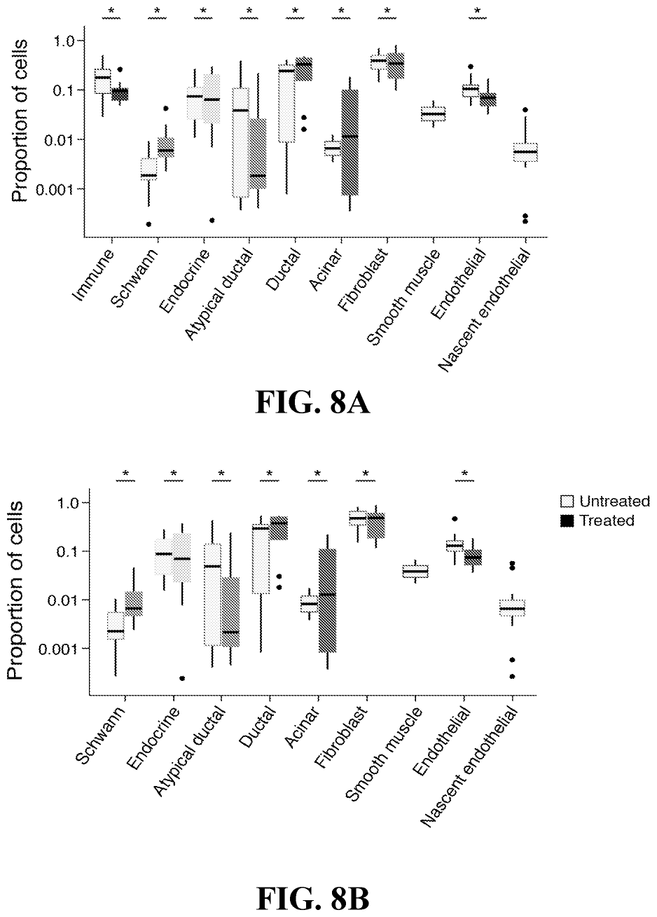

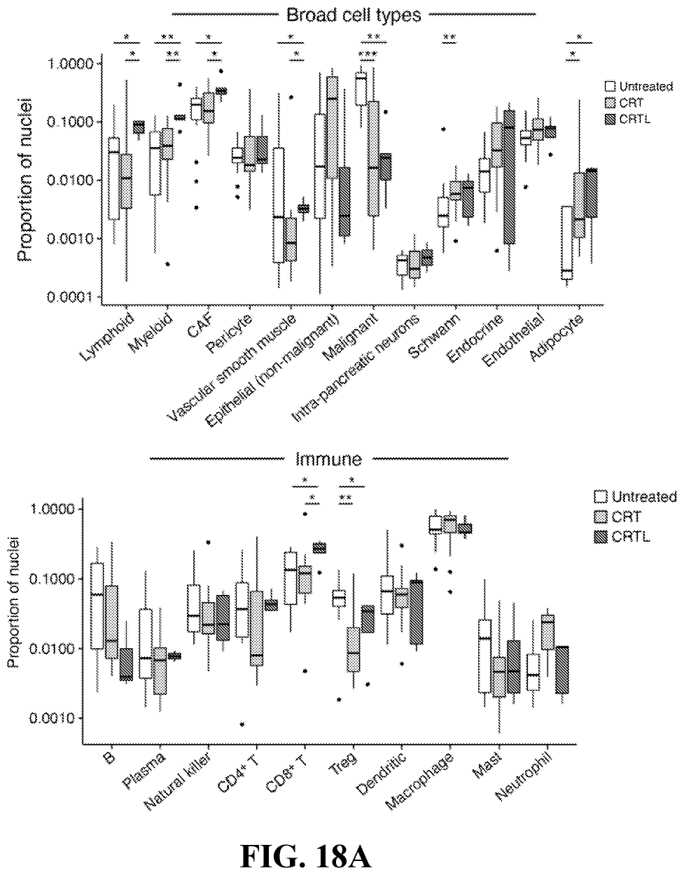

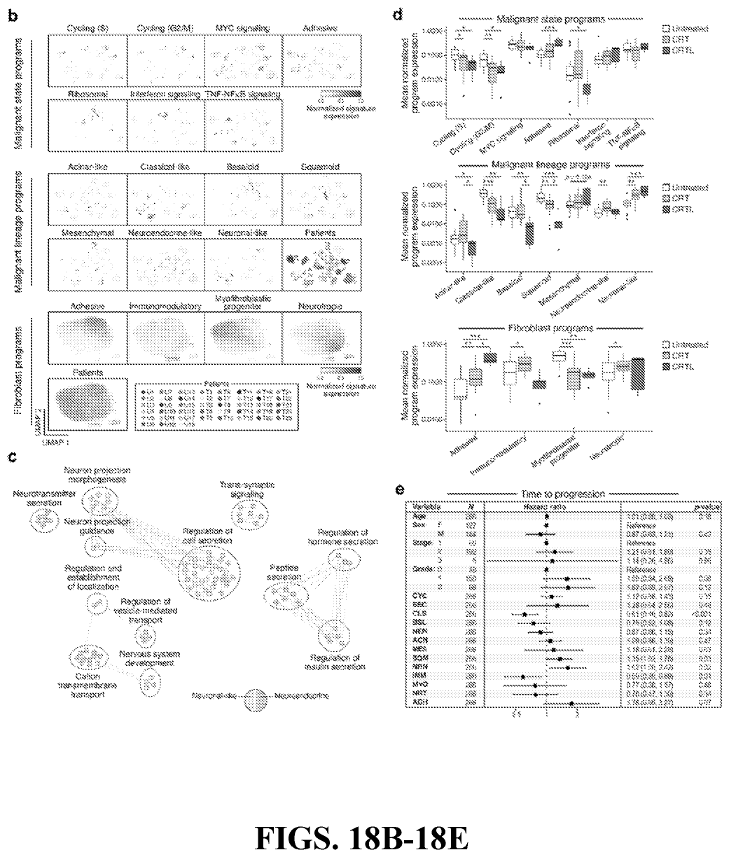

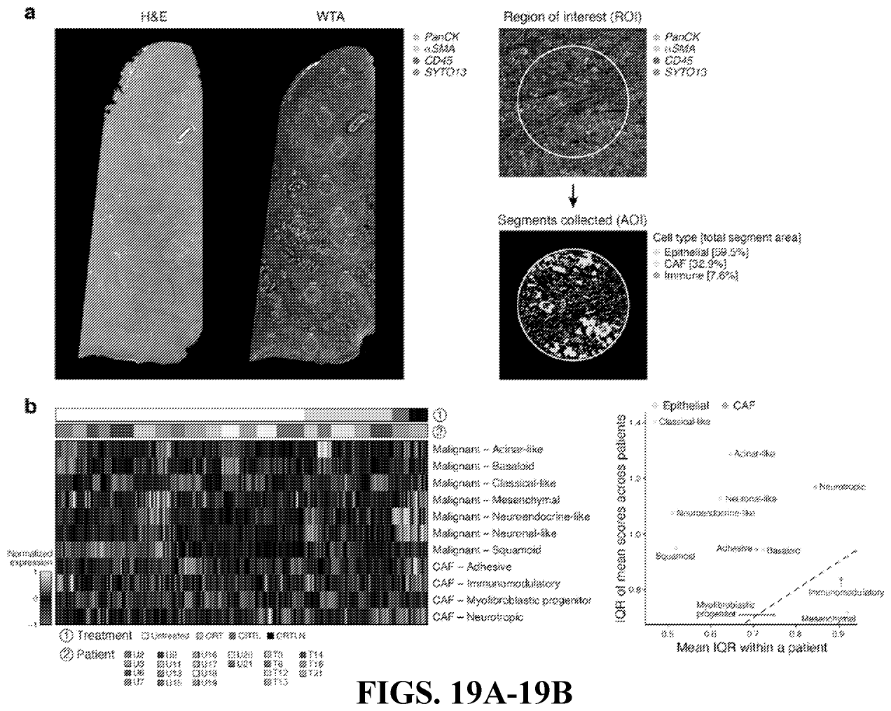

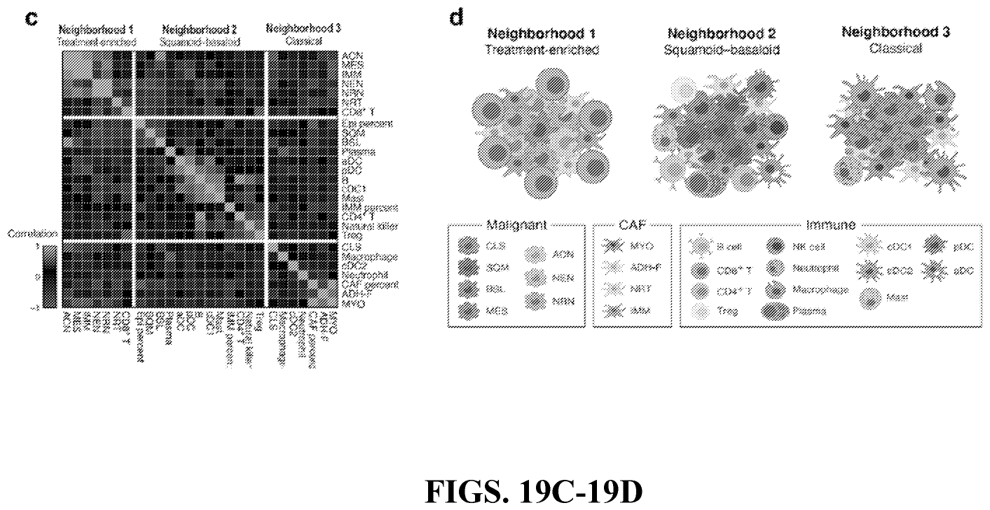

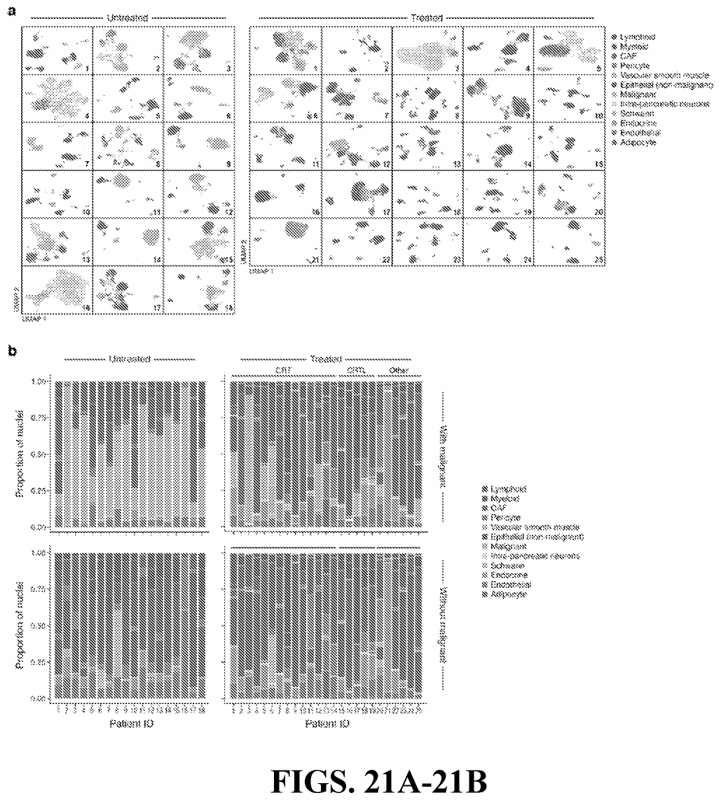

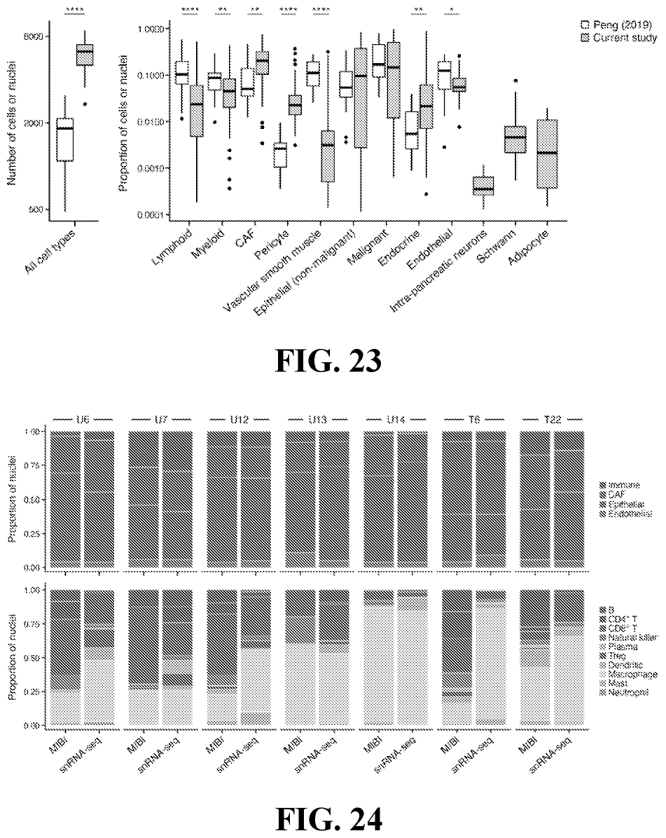

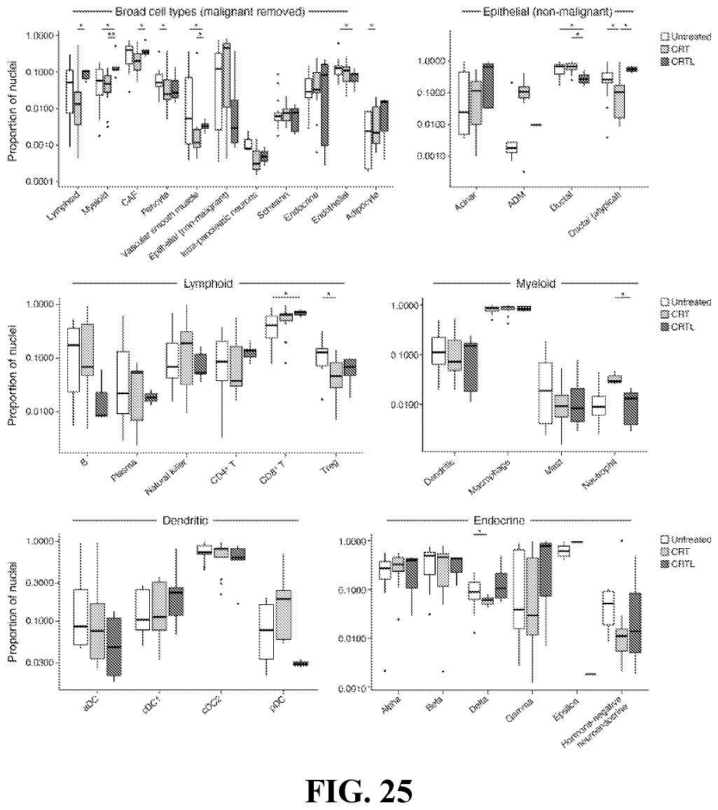

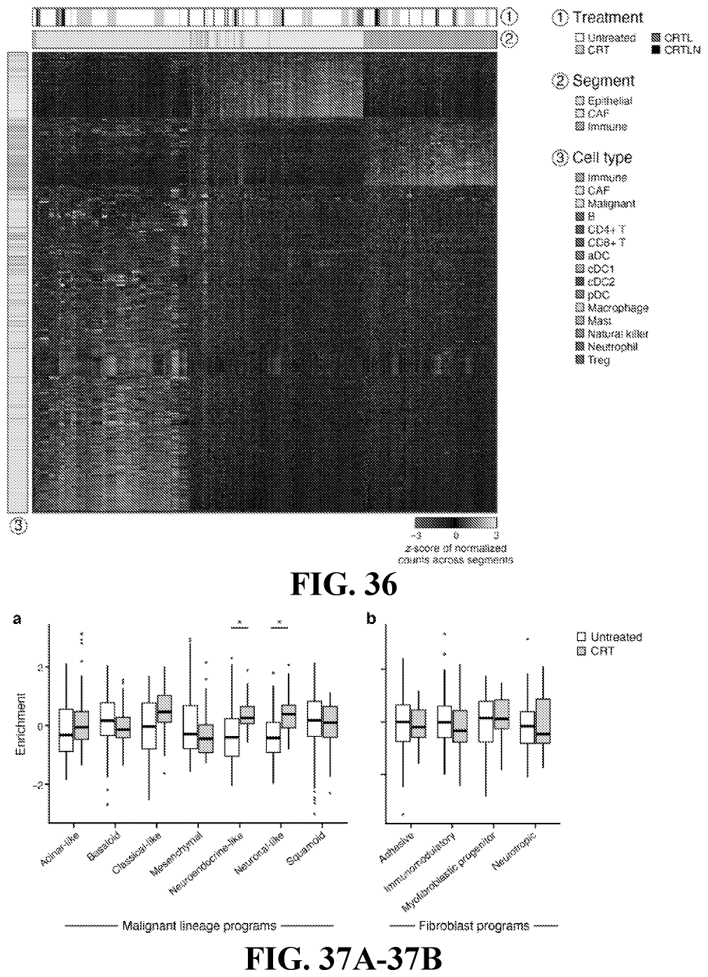

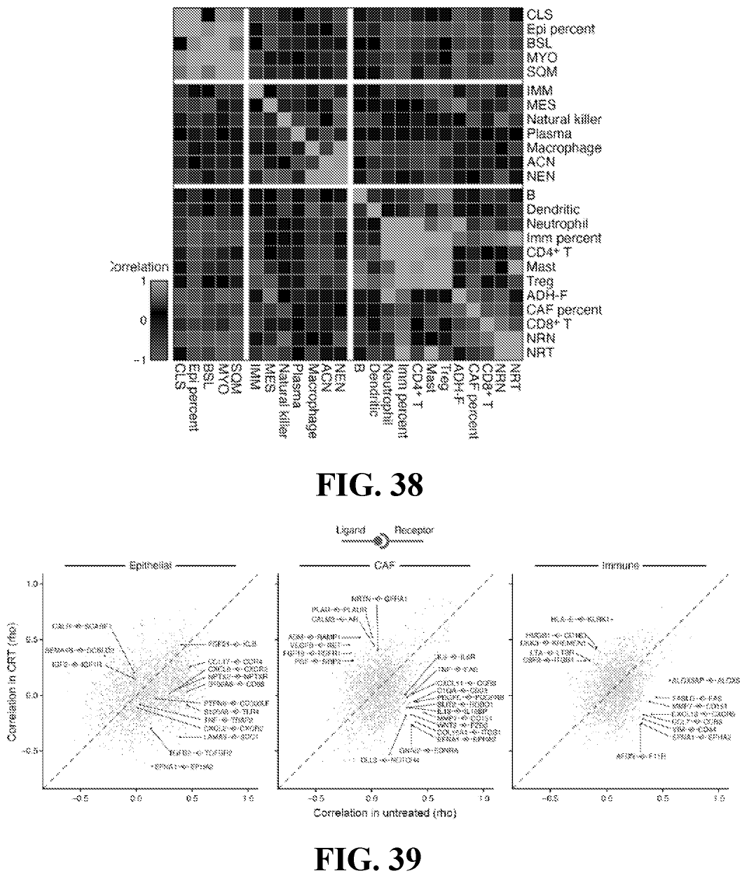

In certain example embodiments, the program and/or signature can include one or more of the following cell types and/or groups thereof Schwann, endocrine, malignant, atypical ductal, ductal, acinar, fibroblast, smooth muscle, endothelial, nascent endothelial, or any combination thereof (See e.g., FIG. 1C), B, plasma, NK, CD4+T, CD8+T, Regulatory T, pDC, cDC1, cDC2, mregDC, macrophage, mast, or any combination thereof (see e.g., FIG. 1C) alpha, beta, delta, gamma (see e.g., FIG. 1C), acinar, acinar-REG+, or both (see e.g., FIG. 1C), Immune, malignant/ductal, fibroblast, endothelial, or any combination thereof (see e.g., FIG. 1D), immune, schwann, endocrine, malignant, atypical ductal, ductal, acinar, fibroblast, smooth muscle, endothelial, nascent endothelial, or any combination thereof (see e.g., FIGS. 6A and 8A-8B); Lymphoid: B, CD4+T, CD8+T, Natural killer, Plasma, Treg (see e.g., FIG. 17B); Myeloid: Dendritic, macrophage, mast, neutrophil, CAF, pericyte, vascular smooth muscle (see e.g., FIG. 17B); Epithelial: acinar, ductal, ADM, ductal (atypical), malignant, intra-pancreatic neurons, Schwann, endocrine (see e.g., FIG. 17B); Endothelial: endothelial (vascular), endothelial (lymphatic), adipocyte (see e.g., FIG. 17B); lymphoid, myeloid, CAF, pericyte, vascular smooth muscle, epithelial (non-malignant), malignant, intra-pancreatic neurons, Schwann, endocrine, endothelial, adipocyte (see e.g., FIG. 17D); Lymphoid: B, CD4+T, CD8+T, Natural killer, Plasma, Treg (see e.g., FIG. 17D); Endothelial: lymphatic and vascular (see e.g., FIG. 17D); Epithelial (non-malignant): acinar, acinar (REG+), ADM, Ductal, Ductal (atypical) (see e.g., FIG. 17D); Myeloid: aDC, aDC1, aDC2, pDC, macrophage, mast, neutrophil (see e.g., FIG. 17D); Endocrine: alpha, beta, epsilon, gamma, hormone-negative neuroendocrine; Broad cell types: lymphoid, myeloid, CAF, pericyte, vascular smooth muscle, epithelial (non-malignant), malignant, intra-pancreatic, Schwann, endocrine, endothelial, adipocyte (see e.g., FIG. 18A); Immune: B, plasma, natural killer, CD4+T, CD8+T, Treg, Dendritic, macrophage, mast, neutrophil (see e.g., FIG. 18A); ACN, MES, IMM, NEN, NRN, NRT, CD8+T, Epi percent, SQM, BSL, Plasma, aDC, pDC, B, cDC1, Mast, IMM percent, CD4+T, natural killer, Treg, CLS, macrophage, cDC2, neutrophil, CAF percent, ADH-F, MYO, or any combination thereof (see e.g., FIG. 19C); lymphoid, myeloid, CAF, pericyte, vascular smooth muscle, epithelial (non-malignant), intra-pancreatic neurons, Schwann, endocrine, endothelial, adipocyte, and combinations thereof (see e.g., FIGS. 21, 23); immune, CAF, epithelial, endothelial (see e.g., FIG. 24); B, CD4+T, CD8+T, natural killer, plasma, Treg, dendritic, macrophage, mast, neutrophil (see e.g., FIG. 24); Broad cell types: lymphoid, myeloid, CAF, pericyte, vascular smooth muscle, epithelial (non-malignant), malignant, intra-pancreatic, Schwann, endocrine, endothelial, adipocyte (see e.g., FIG. 25); lymphoid: B, plasma, natural killer, CD4+T, CD8+T, Treg (see e.g., FIG. 25); dendritic: aDC, aDC1, aDC2, pDC (see e.g., FIG. 25); epithelial (non-malignant): acinar, ADM, Ductal, ductal (Atypical) (see e.g., FIG. 25); myeloid: dendritic, macrophage, mast, neutrophil (see e.g., FIG. 25); endocrine: alpha, beta, delta, gamma, epsilon, hormone-negative neuroendocrine (see e.g., FIG. 25); immune, CAF, malignant, B, CD4+T, CD8+T, Treg aDC, cDC1, cDC2, pDC, macrophage, mast, natural killer, neutrophil (see e.g., FIG. 36); CLS, epi percent, BSL, MyO, SQM, IMM, MES, natural killer, plasma, macrophage, ACN, NEN, B, dendritic, neutrophil, imm percent, CD4+T, mast, Treg, ADH-F, CAF percent, CD8+T, NRN, NRT (see e.g., FIG. 38) or any combination thereof.

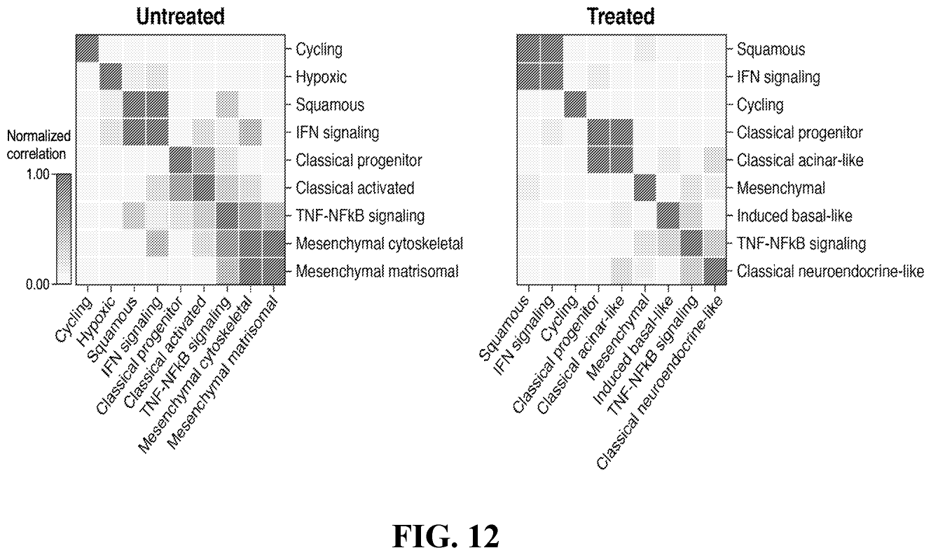

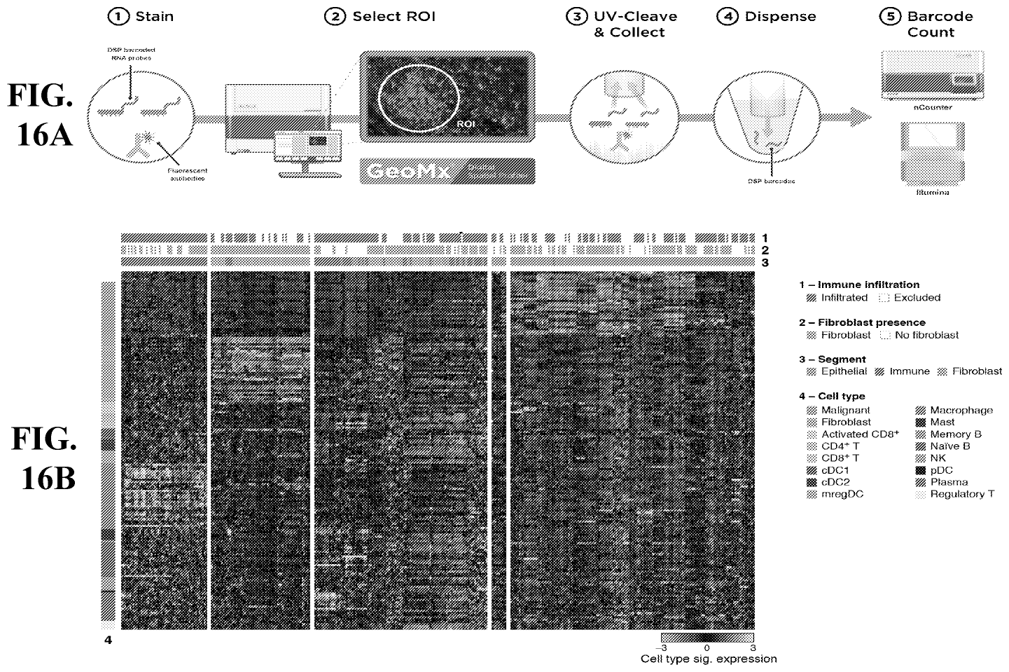

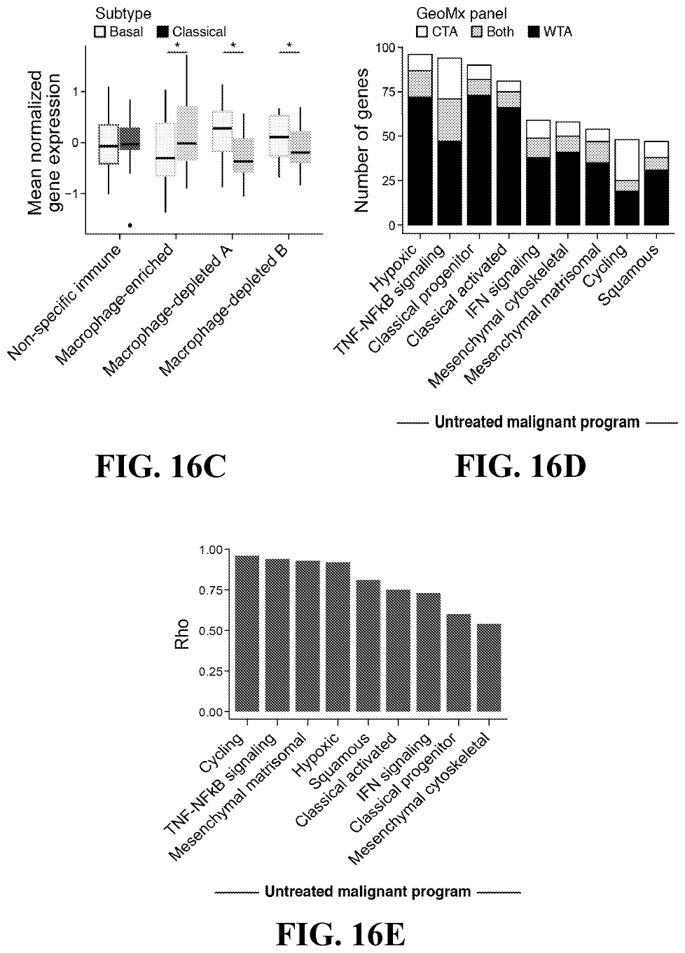

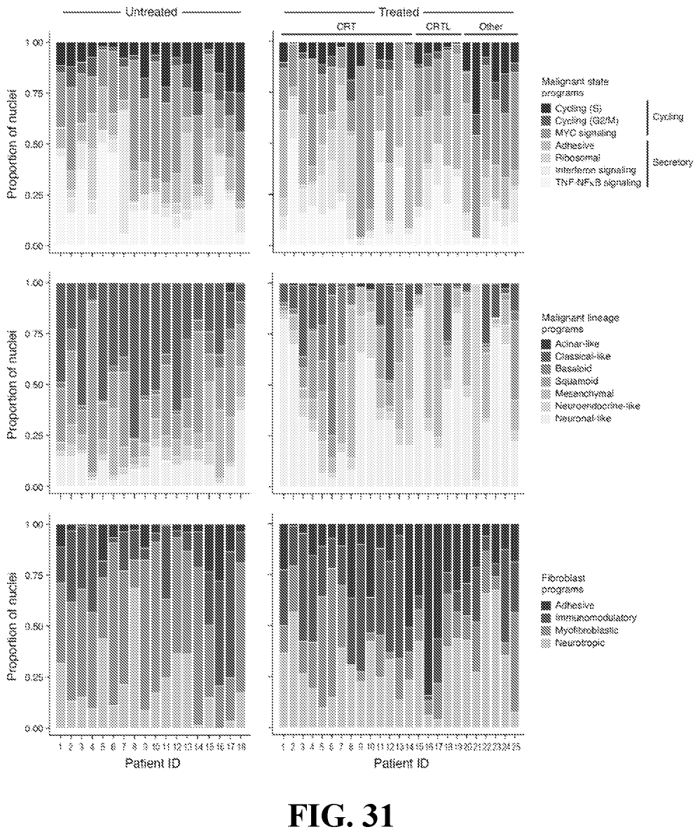

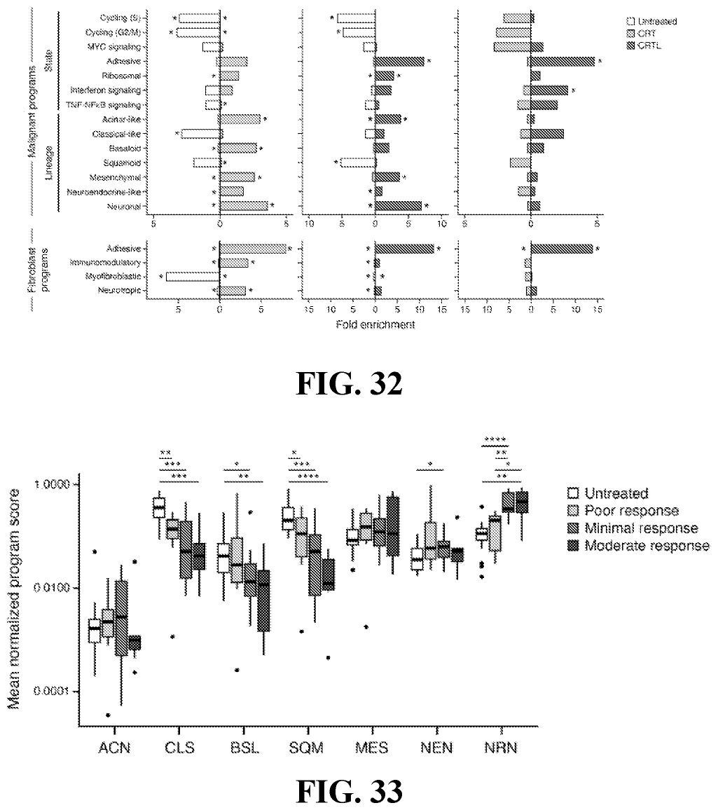

In certain example embodiments, the PDAC tumor program and/or signature includes or is any one or more of the following: GO homeostatic process, GO detoxification, Reactome interferon signaling, Browne interferon responsive genes, Reacctome interferon alpha beta signaling, Hallmark interferon gamma response, GO response to type I interferon, Einav interferon signature in cancer, Hecker IFNB1 targets (See e.g., FIG. 2C), contractility/neuromuscular program, mesodermal development program, Altered differentiation program, M1 program, M2 program, M0 program, or any combination thereof (FIG. 2D), Mesodermal progenitor, secretory, neurotropic, myofibroblast, or any combination thereof (See e.g., FIG. 4D), Hypoxic, interferon signaling, TNF NFκB signaling, mesenchymal matrisomal, classical activated, classical progenitor, squamous, mesenchymal cytoskeletal, cycling, or any combination thereof (see e.g. FIG. 4D), Cycling, Hypoxic, squamous, IFN signaling, Classical progenitor, classical activated, TNF-NFkB signaling, Mesenchymal cytoskeleton, mesenchymal matrisomal, or any combination thereof (See e.g., FIG. 12); GO smooth muscle contraction, GO circulatory system development, GO heart development, GO muscle structure development, Hallmark myogenesis, GO contractile fiber, GO muscle contraction, GO muscle system process, or any combination thereof (see FIG. 15A), myofibroblast, secretory, neurotropic, mesodermal progenitor, neuromuscular, and combinations thereof (FIG. 15B), Neurotropic, mesodermal progenitor, myofibroblast, secretory, or any combination thereof, (see e.g., FIG. 15C), myofibroblast, secretory, neuromuscular, neurotropic (see e.g., FIG. 15C), neurotropic, mesodermal progenitor, secretory, myofibroblast, or any combination thereof (FIG. 15D), myofibroblast, secretory, neurotropic, neuromuscular (see e.g., FIG. 15D), non-specific immune, macrophage-enriched, macrophage-depleted A, macrophage-depleted B (see e.g., FIG. 16C); Malignant cell state expression programs: cycling (S), cycling (G2M), MYC signaling, adhesive, ribosomal, interferon signaling, TNF-NFkappaB signaling (see e.g., FIG. 18A); Malignant lineage programs: acinar-like, classical-like, basaloid, squamoid, mesenchymal, neuroendorcrine-like, neuoronal-like (see e.g., FIG. 18A); Fibroblast expression programs: adhesive, immunomodulatory, myofibroblastic progenitor, neurotropic see e.g., FIG. 18A); state program: RIB, ADH-M, TNF, IFN, MYC, CYS, CYG (see e.g., FIG. 30A); lineage program: NEN, BSL, MES, CAN, SQM, CLS, NRN (see e.g., FIG. 30A); fibroblast programs: ADH-F, MYO, IMM, NRT (see e.g., FIG. 30A); malignant state program: cycling (S), cycling (G2M), MYC signaling, adhesive, ribosomal, interferon signaling, TNF-NFkappaB signaling (see e.g., FIG. 31); malignant lineage programs: acinar-like, classical-like, basaloid, squamoid, mesenchymal, neuroendocrine-like, neuronal-like (see e.g., FIG. 31); fibroblast programs: adhesive, immunomodulatory, myofibroblastic, neutropic (see e.g., FIG. 31); malignant programs: cycling (S), cycling (G2M), MYC signaling, adhesive, ribosomal, interferon signaling, TNF-NFkappaB signaling, acinar-like, classical-like, basaloid, squamoid, mesenchymal, neuroendocrine-like, neuronal (see e.g., FIG. 32); acinar-like, classical-like, basaloid, mesenchymal, neuronal-like, squamoid (see e.g., FIG. 37); adhesive, immunomodulatory, myofibroblastic progenitor, neurotropic (see e.g., FIG. 37), or any combination thereof.

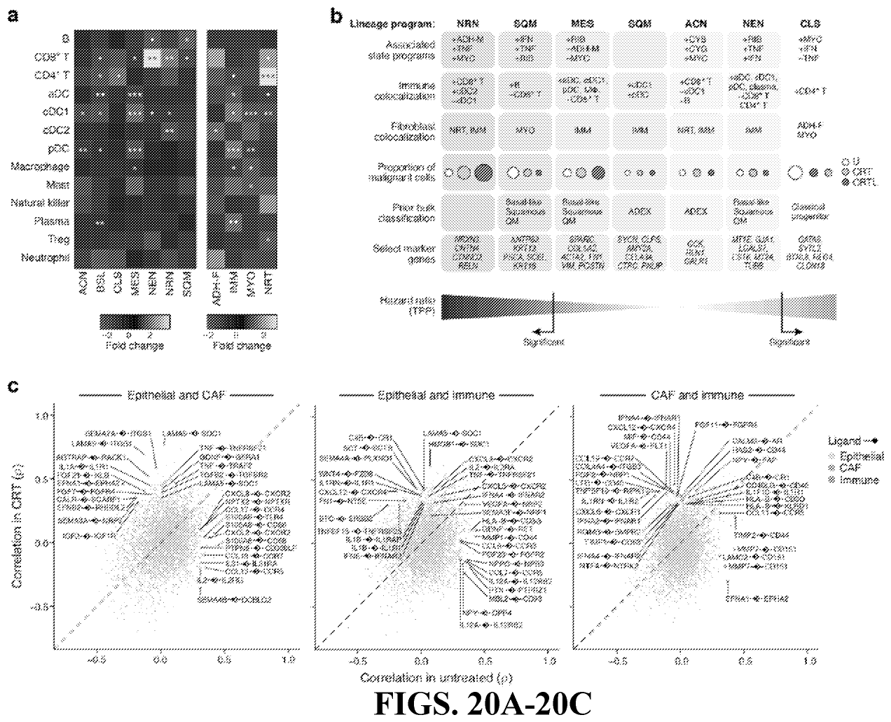

In some embodiments, the co-expressed receptor-ligand pair is selected from: Epithelial and CAF: SEMA7A and ITGB1, LAMA5 and ITGB1, AGTRAP and RACK1, IL1A and IL1R1, FGF21 and EPHA2, CALR and SCARF1, EFNB2 and RHBDL2, SEMA3A and NRP2, IGF2 and IGF1R, LAMA5 and SDC1, TNF and TNFRSF21, GDNF and. GFRA1, TNF and TRAF2, TGFB2 and TGFBR2, LAMA5 and SDC1 (see e.g., FIG. 20C); Epithelial and CAF: CXCL8 and CXCR2, NPTX2 and NPTXR, CCL17 and CCR4, S100A8 and TLR4, S100A8 and CD68, CXCL2 and CXCR2, S100A8 and CD68, PTPN6 and CD300LF, CCL19 and CCR7, IL31 and IL31RA, CCL13 and CCR5, IL2 and IL2RG, SEMA4B and DCBLD2 (see e.g., FIG. 20C); Epithelial and immune: C4B and CR1, SCT and SCTR, SEMA4A and PLXND1, WNT4 and FZD8, IL1RN and IL1R1, CXCL12 and CXCR4, FN1 and NT5E, BTC and ERBB2, TNFSF15 and TNFRSF25, IL1B and IL1RAP, IL1B and IL1R1, IFNE and IFNAR2, LAMA5 and SDC1, HMGB1 and SDC1, CXCL3 and CXCR2, IL2 and IL2RA, TNF and TNFRSF21, CXCL5 and CXCR2, IFNA4 and IFNAR2, VEGFA and NRP2, SEMA3F and NRP1 (see e.g., FIG. 20C); Epithelial and immune: HLA-B and CD3G, GDNF and RET, MMP1 and CD44, CCL8 and CCR3, FGF23 and FGFR2, NPPC and NPR3, CCL7 and CCR5, IL12A and IL12RB2, PTN and PTPRZ1, MBL2 and CD93, NPY and DPP4, IL12A and IL12RB2 (see e.g., FIG. 20C); CAF and immune: IFNA4 and IFNAR1, CXCL12 and CXCR4, MIF and CD44, VEGFA and FLT1, CCL19 and CCR7, COL4A4 and ITGB3, FGF2 and NRP1, LTB and CD40, TNFSF10 and RIPK1, IL1RN and IL1R2, CXCL6 and CXCR1, IFNA2 and IFNAR1, RGMB and BMPR2, TIMP1 and CD63, IFNA4 and NTRK2, FGF11 and FGFR4, CALM3 and AR, HAS2 and CD44, NPY and FAP, C4B and CR1, CD40LG and CD40, IL1F10 and IL1R1, HLA-B and CD3D, HLA-B and KLRD1, CCL11 and CCR5, TIMP2 and CD44, MMP7 and CD151, LAMC2 and CD151, MMP7 and CD151, EFNA1 and EPHA2 (see e.g., FIG. 20C); epithelial: CALR and SCARF1, SEMA4B amd DCBLD2, IGF2 and IGFR1, FGF21 and KLB, CCL17 and CCR4, CXCL8 and CXCR2, NPTX2 and NPTXR, PTPN6 and CD300LF, S100A8 and CD68, TNF and TRAF2, CXCL2 and CXCR2, LAMA5 and SDC1, TGFB2 and TGFBR2, EFNA1 and EPHA2 (see e.g., FIG. 39); CAF: PGF and NRP2, FGF19 and FGFR1, VEGFB and RET, ADM and RAMP1, CALM3 amd AR, PLAU and PLAUR, NRTN and GFRA1, IL6 and IL6R, TNF and FAS, CXCL11 and CCR3, CIQA and CD93, PDGFC and PDGFRB, SLIT2 and ROBO1, IL18 and IL18BP, MMP7 and CD151, WNT2 and FZD3, COL16A1 and ITGB1, EFNA1 and EPHA2, GNAI2 and EDNRA, DLL3 and NOTCH4 (see e.g., FIG. 39); CSF2 and ITGB1, LTA and LTBR, DKK3 and KREMEN1, HMGB1 and CD163, HLA-E and KLRK1, ALOX5AP and ALOX5, FASLG and FAS, MMP7 and CD151, CXCL13 and CXCR5, CCL7 and CCR3, VIM and CD44, EFNA1 and EPHA2, AFDN and F1 IR (see e.g., FIG. 39); and combinations thereof.

Reference to any of Tables 2.1-2.6 herein also makes reference to Extended Data Table 2 of W. Hwang and K. Jagadeesh et al., (2020) Single-nucleus and spatial transcriptomics of archival pancreatic cancer reveals multi-compartment reprogramming after neoadjuvant treatment, BioRxiV. 2020. https://doi.org/10.1101/2020.08.25.267336.

In certain example embodiments, the therapeutic, diagnostic, and screening methods disclosed herein target, detect, or otherwise make use of one or more biomarkers of an expression signature. As used herein, the term “biomarker” can refer to a gene, an mRNA, cDNA, an antisense transcript, a miRNA, a polypeptide, a protein, a protein fragment, or any other nucleic acid sequence or polypeptide sequence that indicates either gene expression levels or protein production levels. Accordingly, it should be understood that reference to a “signature” in the context of those embodiments may encompass any biomarker or biomarkers whose expression profile or whose occurrence is associated with a specific cell type, subtype, or cell state of a specific cell type or subtype within a population of cells (e.g., Synovial Sarcoma cells) or a specific biological program. As used herein the term “module” or “biological program” can be used interchangeably with “expression program” and refers to a set of biomarkers that share a role in a biological function (e.g., an activation program, cell differentiation program, proliferation program). Biological programs can include a pattern of biomarker expression that result in a corresponding physiological event or phenotypic trait. Biological programs can include up to several hundred biomarkers that are expressed in a spatially and temporally controlled fashion. Expression of individual biomarkers can be shared between biological programs. Expression of individual biomarkers can be shared among different single cell types; however, expression of a biological program may be cell type specific or temporally specific (e.g., the biological program is expressed in a cell type at a specific time). Expression of a biological program may be regulated by a master switch, such as a nuclear receptor or transcription factor. As used herein, the term “topic” refers to a biological program. Topics are described further herein. The biological program (topic) can be modeled as a distribution over expressed biomarkers.

In certain embodiments, the expression of the signatures disclosed herein (e.g., core oncogenic signature) is dependent on epigenetic modification of the biomarkers or regulatory elements associated with the signatures (e.g., chromatin modifications or chromatin accessibility). Thus, in certain embodiments, use of signature biomarkers includes epigenetic modifications of the biomarkers that may be detected or modulated. As used herein, the terms “signature”, “expression profile”, or “expression program” may be used interchangeably (e.g., expression of genes, expression of gene products or polypeptides). It is to be understood that also when referring to proteins (e.g., differentially expressed proteins), such may fall within the definition of “gene” signature. Levels of expression or activity may be compared between different cells in order to characterize or identify for instance signatures specific for cell (sub)populations. Increased or decreased expression or activity or prevalence of signature biomarkers may be compared between different cells in order to characterize or identify for instance specific cell (sub)populations. The detection of a signature in single cells may be used to identify and quantitate, for instance, specific cell (sub)populations. A signature may include a biomarker whose expression or occurrence is specific to a cell (sub)population, such that expression or occurrence is exclusive to the cell (sub)population. An expression signature as used herein, may thus refer to any set of up- and/or down-regulated biomarkers that are representative of a cell type or subtype. An expression signature as used herein, may also refer to any set of up- and/or down-regulated biomarkers between different cells or cell (sub)populations derived from a gene-expression profile. For example, an expression signature may comprise a list of biomarkers differentially expressed in a distinction of interest. A signature can also include a cell type and/or cell state distribution. The cell type distribution can, for example, be indicative of the state of a population of cells or tissue, such as a tumor tissue, and/or a microenvironment of a tissue or population of cells, and/or a niche microenvironment within a tissue or cell population. Cell type

The signature according to certain embodiments of the present invention may comprise or consist of one or more biomarkers, such as for instance 1, 2, 3, 4, 5, 6, 7, 8, 9, 10 or more. In certain embodiments, the signature may comprise or consist of two or more biomarkers, such as for instance 2, 3, 4, 5, 6, 7, 8, 9, 10 or more. In certain embodiments, the signature may comprise or consist of three or more biomarkers, such as for instance 3, 4, 5, 6, 7, 8, 9, 10 or more. In certain embodiments, the signature may comprise or consist of four or more biomarkers, such as for instance 4, 5, 6, 7, 8, 9, 10 or more. In certain embodiments, the signature may comprise or consist of five or more biomarkers, such as for instance 5, 6, 7, 8, 9, 10 or more. In certain embodiments, the signature may comprise or consist of six or more biomarkers for instance 6, 7, 8, 9, 10 or more. In certain embodiments, the signature may comprise or consist of seven or more biomarkers, such as for instance 7, 8, 9, 10 or more. In certain embodiments, the signature may comprise or consist of eight or more biomarkers, such as for instance 8, 9, 10 or more. In certain embodiments, the signature may comprise or consist of nine or more biomarkers, such as for instance 9, 10 or more. In certain embodiments, the signature may comprise or consist of ten or more biomarkers, such as for instance 10, 11, 12, 13, 14, 15, or more. It is to be understood that a signature according to the invention may for instance also include different types of biomarkers combined (e.g., genes and proteins).

In certain embodiments, a signature is characterized as being specific for a particular cell or cell (sub)population if it is upregulated or only present, detected or detectable in that particular cell or cell (sub)population, or alternatively is downregulated or only absent, or undetectable in that particular cell or cell (sub)population. In this context, a signature consists of one or more differentially expressed genes/proteins or differential epigenetic elements when comparing different cells or cell (sub)populations, including comparing different cells or cell (sub)populations (e.g., synovial sarcoma cells), as well as comparing malignant cells or malignant cell (sub)populations with other non-malignant cells or non-malignant cell (sub)populations. It is to be understood that “differentially expressed” biomarkers include biomarkers which are up- or down-regulated as well as biomarkers which are turned on or off. When referring to up-or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art. Differential expression of biomarkers may also be determined by comparing expression of biomarkers in a population of cells or in a single cell. In certain embodiments, expression of one or more biomarkers is mutually exclusive in cells having a different cell state or subtype (e.g., two genes are not expressed at the same time). In certain embodiments, a specific signature may have one or more biomarkers upregulated or downregulated as compared to other biomarkers in the signature within a single cell. In certain embodiments, a specific signature may have one or more biomarkers upregulated or downregulated as compared to other biomarkers in the signature within a single nucleus within a cell. Thus, a cell type or subtype can be determined by determining the pattern of expression in a single cell and/or a single nucleus within a cell.

As discussed herein, differentially expressed biomarkers may be differentially expressed on a single cell level or may be differentially expressed on a cell population level. Preferably, the differentially expressed biomarkers as discussed herein, such as constituting the expression signatures as discussed herein, when as to the cell population level, refer to biomarkers that are differentially expressed in all or substantially all cells of the population (such as at least 80%, preferably at least 90%, such as at least 95% of the individual cells). This allows one to define a particular subpopulation of cells. As referred to herein, a “subpopulation” of cells preferably refers to a particular subset of cells of a particular cell type (e.g., Synovial Sarcoma) which can be distinguished or are uniquely identifiable and set apart from other cells of this cell type. The cell subpopulation may be phenotypically characterized and is preferably characterized by the signature as discussed herein. A cell (sub)population as referred to herein may constitute of a (sub)population of cells of a particular cell type characterized by a specific cell state.

When referring to induction, or alternatively suppression of a particular signature, preferable is meant induction or alternatively suppression (or upregulation or downregulation) of at least one biomarker of the signature, such as for instance at least two, at least three, at least four, at least five, at least six, or all biomarkers of the signature.

Example gene signatures and topics are further described below.

### Malignant Programs

In some embodiments the PDAC tumor signature and/or program is or includes a malignant signature and/or program. In some embodiments, the malignant signature and/or program is or includes of a neoadjuvant treated signature and/or program. In some embodiments, the malignant signature is or includes of a neoadjuvant untreated signature and/or program. In certain embodiments, a malignant signature (e.g., signature of differentially expressed genes between malignant cells and non-malignant cells, e.g. epithelial cells, CAFs, CD8 and CD4 T cells, B cells, NK cells, macrophages, or mastocytes) comprises one or more biomarkers, expression programs, biologic programs, receptor-ligand interactions, cell state distribution, cell type distribution, or any combination thereof as in any of 1B-1D, 2A-2D, 3A-3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, Tables 2.1-2.6, 3, 4, and any combination thereof.

In some embodiments, the malignant signature is associated with a specific immune microniche signature. Tumor niche microenvironment signatures are also described in greater detail elsewhere herein.

### Malignant Neoadjuvant Treated and Untreated Programs

In some embodiments, the malignant signature and/or program is or includes a neoadjuvant treated malignant signature and/or program (i.e., a signature specific to malignant cells that have undergone a neoadjuvant treatment). In some embodiments, the malignant signature and/or program is or includes a neoadjuvant untreated malignant signature (i.e., a signature specific to malignant cells that have not undergone a neoadjuvant treatment).

In some embodiments, the neoadjuvant treated malignant signature and/or program comprises one or more biomarkers, expression programs, biologic programs, receptor-ligand interactions, cell state distribution, cell type distribution, or any combination thereof as in any of 1B-1D, 2A-2D, 3A-3C, 4A-4C, 5A-5C, 7, 9A-9D, 10, 11, 12, 14, 15A-15D, 17B-17G, 18A-18D, 19A-19D, 20A-20C, 21A-21B, 23, 24, 25, 26, 26, 29, 30, 31, 32, 36, 37, 38, 39, Table 2.1-2.6, and any combination thereof.

In some embodiments, the neoadjuvant untreated malignant and/or program signature comprises one or more biomarkers, expression programs, biologic programs, receptor-ligand interactions, cell state distribution, cell type distribution, or any combination thereof as in any of 1B-1D, 2A-2D, 3A-3C, 4A-4C, 5A-5C, 7, 9A-9D, 10, 11, 12, 14, 15A-15D, 17B-17G, 18A-18D, 19A-19D, 20A-20C, 21A-21B, 23, 24, 25, 26, 26, 29, 30, 31, 32, 36, 37, 38, 39, Table 2.1-2.6, and any combination thereof.

In some embodiments, neoadjuvant treatment can result in a shift from a classical-like program to a basal-like program. In some embodiments, the neoadjuvant treated malignant signature is associated with a specific immune microniche signature and/or program. In some embodiments, the neoadjuvant untreated malignant signature is associated with a specific immune microniche signature and/or program. Tumor niche microenvironment signatures are also described in greater detail elsewhere herein.

### Cancer Associate Fibroblast (CAF) Programs

In some embodiments the PDAC tumor signature is or includes a CAF signature and/or program. In some embodiments, the CAF signature and/or program comprises one or more biomarkers, expression programs, biologic programs, receptor-ligand interactions, cell state distribution, cell type distribution, or any combination thereof as in any of FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

In some embodiments, the CAF signature and/or program is associated with a specific immune microniche signature. Tumor niche microenvironment signatures and/or programs are also described in greater detail elsewhere herein.

### CAF Neoadjuvant Treated and Untreated Programs

In some embodiments, the CAF signature and/or program is or includes a neoadjuvant treated CAF signature and/or program (i.e., a signature specific and/or program to CAFs that have undergone a neoadjuvant treatment). In some embodiments, the malignant signature is or includes a neoadjuvant untreated malignant signature (i.e., a signature specific and/or program to CAFs that have not undergone a neoadjuvant treatment).

In some embodiments, the neoadjuvant treated CAF signature and/or program comprises one or more biomarkers, expression programs, biologic programs, receptor-ligand interactions, cell state distribution, cell type distribution, or any combination thereof as in any of 1B-1D, 2A-2D, 3A-3C, 4A-4C, 5A-5C, 7, 9A-9D, 10, 11, 12, 14, 15A-15D, 17B-17G, 18A-18D, 19A-19D, 20A-20C, 21A-21B, 23, 24, 25, 26, 26, 29, 30, 31, 32, 36, 37, 38, 39, Table 2.1-2.6, and any combination thereof.

In some embodiments, the neoadjuvant untreated CAF signature and/or program comprises one or more biomarkers, expression programs, biologic programs, receptor-ligand interactions, cell state distribution, cell type distribution, or any combination thereof as in any of 1B-1D, 2A-2D, 3A-3C, 4A-4C, 5A-5C, 7, 9A-9D, 10, 11, 12, 14, 15A-15D, 17B-17G, 18A-18D, 19A-19D, 20A-20C, 21A-21B, 23, 24, 25, 26, 26, 29, 30, 31, 32, 36, 37, 38, 39, Table 2.1-2.6, and any combination thereof.

In some embodiments, the CAF treated malignant signature and/or program is associated with a specific immune microniche signature. In some embodiments, the neoadjuvant untreated CAF signature is associated with a specific immune microniche signature. Tumor niche microenvironment signatures are also described in greater detail elsewhere herein.

### Immune Microniche Signatures

As demonstrated in the Working Examples elsewhere herein, different microinches can have different immune signatures. In some embodiments, the PDAC immune microniche signature and/or program one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39, Table 7, or any combination thereof.

### Methods of Detecting Expression Signatures and Programs

Described herein are methods of detecting one or more signatures and/or programs, such as a PDAC signature and/pr program, in one or more tissues and/or cells of a subject. In some embodiments, a PDAC signature and/or program is detected in a single cell of a PDAC tumor. In some embodiments, a PDAC signature and/or program is detected in a single nucleus of a PDAC tumor cell or PDAC tumor-associated cell. In some embodiments, a tumor-associated cell is an immune cell present in a tumor microenvironment (i.e., the microenvironment surrounding the tumor in situ) and/or tumor niche local microenvironment (i.e. a specific region or compartment within a tumor). PDAC signatures and/or programs that can be detected in various embodiments are discussed and described in greater detail elsewhere herein.

In one embodiment, the signature's and/or program's genes, biomarkers, and/or cells may be detected or isolated by immunofluorescence, immunohistochemistry (IHC), fluorescence activated cell sorting (FACS), mass spectrometry (MS), mass cytometry (CyTOF), any gene or transcript sequencing method, including but not limited to, RNA-seq, single cell RNA-seq, single nucleus RNAseq, spatial transcriptomics, spatial proteomics, quantitative RT-PCR, single cell qPCR, FISH, RNA-FISH, MERFISH (multiplex (in situ) RNA FISH), Nanostring, in situ hybridization, CRISPR-effector system mediated screening assay (e.g. SHERLOCK assay), compressed sensing, and any combination thereof. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 Mar.;26(3):317-25). These and other methods are described in greater detail elsewhere herein (see e.g., the section regarding “methods of diagnosing, prognosing, and/or treating PDAC” and Working Examples herein).

Methods of Diagnosing, Prognosing, and/or Treating PDAC

Described herein are methods of diagnosing, prognosing, and/or treating PDAC in a subject in need thereof. In some embodiments, methods of diagnosing, prognosing, and/or treating PDAC in a subject in need thereof can include detecting one or more PDAC signatures and/or programs, which are described in greater detail elsewhere herein.

### Diagnosing and Prognosing PDAC

Described in certain example embodiments herein are methods of stratifying pancreatic ductal adenocarcinoma (PDAC) patients into treatment groups and/or prognosing PDAC or treatment outcome and/or survival in a patient comprising: detecting, in one or more a PDAC tumor cells, a malignant cell signature, program, or both; a cancer-associated fibroblast (CAF) signature, program, or both; an immune microniche signature, program, a tumor spatial neighborhood; one or more co-expressed receptor-ligand pairs; or any combination thereof; wherein a characteristic regarding a patient's treatment, a patient's response to a treatment, and/or their survival is determined or predicted based on the detection of one or more of the signatures, programs, and/or states.

In certain example embodiments, the malignant cell signature and/or program comprises a lineage specific expression program selected from: a squamous program, a mesenchymal cytoskeletal program, mesenchymal matrisomal program, a classical progenitor program, a classical activated program, or any combination thereof; a lineage specific expression program selected from: a squamous program, a mesenchymal program, an induced basal-like program, a classical progenitor program, a classical acinar-like program, a classical neuroendocrine-like program, or any combination thereof; a cell state specific expression program selected from: a cycling program, a hypoxic program, TNF-NFkB signaling program, an interferon signaling program, or any combination thereof, a cell state specific expression program selected from: a cycling program, a TNF-NFkB signaling program, or an interferon signaling program, or any combination thereof, a neoadjuvant treated malignant cell expression program; an untreated malignant cell expression program, a cell state expression program selected from: a neuronal-like program, a neuroendocrine like program, a mesenchymal program, a squamoid program, a MYC signaling program, a cycling (G2M) program, a cycling (S) program, or any combination thereof; a lineage specific expression program selected from: an acinar-like program, a classical-like program, a basaloid program, a squamoid program, a mesenchymal program, a neuroendocrine like program, a neuronal like program, or any combination thereof, or any combination thereof.

In certain example embodiments, the CAF signature and/or program (a) comprises a myofibroblast program; a neurotropic program; a secretory program; a mesodermal progenitor program a neuromuscular program; or any combination thereof, (b) comprises a neoadjuvant treated CAF signature and/or program selected from: a neuromuscular program, a secretory program, a neurotropic program, or any combination thereof; (c) comprises an untreated CAF signature and/or program selected from: a mesodermal progenitor program, a myofibroblast program, a neurotropic program, a secretory program, or any combination thereof, or (d) comprises an adhesive expression program, an immunomodulatory expression program, a myofibroblastic progenitor expression program, or a neurotropic expression program.

In certain example embodiments, the tumor spatial neighborhood is a treatment enriched neighborhood, a squamoid-basaloid neighborhood, or a classical neighborhood.

In certain example embodiments, the one or more co-expressed receptor-ligand pairs is selected from: an Epithelial compartment—CAF compartment pair; an Epithelial compartment—Immune compartment pair; a CAF compartment and Immune compartment pair; or any combination thereof.

In certain example embodiments, the method comprises, detecting, in one or more a PDAC tumor cells, an untreated tumor malignant cell signature and/or program and an untreated CAF signature and/or program, wherein the untreated tumor malignant cell signature and/or program comprises a lineage specific expression program selected from: a squamous program, a mesenchymal cytoskeletal program, mesenchymal matrisomal program, a classical progenitor program, a classical activated program, or any combination thereof, and the untreated tumor CAF signature and/or program is selected from: a mesodermal progenitor program, a myofibroblast program, a neurotropic program, a secretory program, or any combination thereof.

In certain example embodiments, the method further comprises determining a tumor heterogeneity score for the PDAC tumor, wherein the tumor heterogeneity score is calculated by determining a number of highly expressed programs in the one or more PDAC cells. In some embodiments the number of highly expressed programs is 0, 1, 2, 3, 4 or more. The greater the number of highly expressed programs, the greater the tumor heterogeneity.

In certain example embodiments, the method further comprises assigning the PDAC tumor to a single malignant class and to a single CAF class, wherein the malignant class is selected from A0, A1, A2, S0, S1, S2, C0, C1, C2, M0, M1, M2, P0, P1, or P2, and wherein the CAF class is selected from S0, S1, NO, N1, M0, M1, P0, or P1.

In certain example embodiments, the PDAC tumor is assigned to a combined risk class that integrates the malignant risk group and CAF risk group class and is selected from: a low combined risk group, a low-intermediate combined risk group, a high-intermediate risk group, or a high combined risk group, where a PDAC tumor in a low malignant risk group and in a low CAF risk group is classified into the low combined risk group, a PDAC tumor in a high malignant risk and in a high CAF risk is classified into the high combined risk group, a PDAC tumor in an intermediate malignant risk group or in an intermediate CAF risk and in a high malignant risk or in a high CAF risk is classified into the high-intermediate combined risk group, and a PDAC tumor in a low malignant risk group and in a high CAF risk group, a PDAC tumor in a high malignant risk group and in a low CAF risk group, a PDAC tumor in a low malignant risk group and in a low CAF risk group, a PDAC tumor in an intermediate malignant risk group and in an intermediate CAF risk group, a PDAC tumor in a low malignant risk group and in an intermediate CAF risk group is classified into the low-intermediate combined risk group. A subject with a PDAC tumor in low combined risk group has the greatest likelihood of longest survival.

In certain example embodiments, wherein the malignant cell signature and/or program comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

In certain example embodiments, wherein the CAF cell signature and/or program comprises one or more biomarkers, expression programs, biologic programs, receptor-ligand interactions, cell state distribution, cell type distribution, or any combination thereof as in any of FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

In certain example embodiments, the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39, and Table 7, or any combination thereof.

In certain example embodiments, the CAF signature and/or program comprises one or more of the following genes or groups of genes: keratin, vimentin, CD45, CD31, or any combination thereof (see e.g., FIG. 1D); MSA4A1, CREB3L2, NCR1, CD40LG, PTPRC, CD8B, FOXP3, CD4, ZFAT, XCR1, PLD4, IL12B, CD163, CPA3, SOX10, GCG, IAPP, SST, PPY, KRT19, CFTR, SPINK1, COL1A1, ACTA2, PECAM, SOX18, or any combination thereof (see e.g., FIG. 2A); CD40, CD3G, LRP5, CXCR4, or any combination thereof (see e.g., FIG. 2B); CD40LG, HLA-A, HLA-B, HLA-C, WNT7B, CXCL12, WNT5A, or any combination thereof (see e.g., FIG. 2B); CYP3A5, CTSE, ANXA10, LYZ, BTNL8, GATA6, HNF4A, AS1, CCL20, TFF1, HLA-B, MT2A, IF16, LY6E, SOD2, IL34, IFNAR2, ISG15, IFIT3, MUC16, OAS2, IL1ORB, COMMD7, ZBP1, TP63, WNT7B, GOS2, IFIT2, IFIT1, SCO2, and any combination thereof (see e.g., FIG. 2C); CYP3A5, CTSE, ANXA10, LYZ, BTNL8, GATA6, HNF4A, AS1, CCL20, TFF1, or any combination thereof (see e.g., FIG. 2C); HLA-B, MT2A, IF16, LY6E, SOD2, IL34, IFNAR2, ISG15, IFIT3, MUC16, OAS2, IL1ORB, COMMD7, ZBP1, TP63, WNT7B, GOS2, IFIT2, IFIT1, SCO2, or any combination thereof, (see e.g., FIG. 2C); Type I IFN, Type II IFN, or both (see e.g., FIG. 2D); MHC-I (HLAB), MUC16, WNT7B, TP63, or any combination thereof (see e.g., FIG. 2D; CCL20, GATA6, or both (see e.g., FIG. 2D); MYH11, ACTG2, MYOCD, BCL2, or any combination thereof (see e.g., FIG. 2D); a TGFbeta pathway gene, FAP, PRDM6, ORRX1, RUNX2 or any combination thereof (see e.g., FIG. 2D; SLAMF6, CD69, STAT4, IL7R, ITGAE, ITGA4, or any combination thereof (see e.g., FIG. 2D; ITK, FYN, or both, (see e.g., FIG. 2D); CD86, TNFSF8, TNFRSFIB, IFI44L, LILRB, or any combination thereof (see e.g., FIG. 2D); CD163, MRC1, SIGLEC1, MS4A6A, FES, or any combination thereof(see e.g., FIG. 2D); MARCO, AQP9, CYP27A1, NCF2, or any combination thereof (see e.g., FIG. 2D); PanCK, CD45, alphaSMA, SYTO13, or any combination thereof (see e.g., FIGS. 4A-4B); CPA3, RUNX1, ARIDIB, EWSR1, ILF3, ARID1A, PIK3R1, CDKN1A, ALCAM, NOTCH2, TGFBR1, DAB2, CD68, ITGB2, CD4, CSF1R, CD53, CD81, FCGRT, NFKBIA, ENG, CTNNB1, RAB7A, IFNGR2, TGFBR2, TCL1A, CDC25B, CD8A, CCXCR6, IL11RA, PAX5, SFXN1, TCF3, CASP10, CD79B, CD19, HLA-DOB, PDK1, CD27, CD79A, ADA, FBXW7, FCER2, CD1C, MNAT1, CD40LG, CXCL11, IL15, KLRF1, NLRP3, WNT10B, BCAT1, HLA-DRB1, RASGRF2, S100A8, IL3RA, NT5E, IDO1, CXCL10, CXCL9, TNFRSF4, THEM4, CDC7, IL22RA2, CR2, CXCL13, FOXP3, CTLA4, TIGIT, DUSP5, CD200, DUSP2, TSHR, CRLF2, RASGRF1, LAG3, PDCD1, FCRL2, ITGB7, SH2D1B, BMP6, CD274, IFNG, or any combination thereof (see e.g., FIG. 4F); ENTPD1, ITGAE, GABPB1, AS1, or any combination thereof (see e.g., FIG. 9A); CAMK1D, ELMO1, SLAMF6, CCND3, IQGAP2, ATF7IP2, FL11, ATP8A1, ETS1, AUTS2, GRAP2, PREK1, DOCK10, PDE7A, ZFP36L2, STAT4, MGAT5, IKZF1, CAMK4, DIAPH1, PIK3R1, FYN, EMB, CD96, ITK, ITGA4, CD69, CNOT6L, PRKCQ, SPOCK2, ARHGAP35, PRKX, CMTM7, TGFBR3, BIN2, IL7R, SLA2, CXCR4, P2RY8, CD226, CRACR2A, TNFSF8, or any combination thereof (see e.g., FIG. 9A); ENTPD1, ITGAE, GABPB1, AS1, CAMK1D, ELMO1, SLAMF6, CCND3, IQGAP2, ATF7IP2, FL11, ATP8A1, ETS1, AUTS2, GRAP2, PREK1, DOCK10, PDE7A, ZFP36L2, STAT4, MGAT5, IKZF1, CAMK4, DIAPH1, PIK3R1, FYN, EMB, CD96, ITK, ITGA4, CD69, CNOT6L, PRKCQ, SPOCK2, ARHGAP35, PRKX, CMTM7, TGFBR3, BIN2, IL7R, SLA2, CXCR4, P2RY8, CD226, CRACR2A, TNFSF8, or any combination thereof, (see e.g., FIG. 9A); MMP1, MMP12, AQP9, NCF2, TIMP1, MARCO, CD74, TGM2, TGFB1, LGALS3, or any combination thereof (see e.g., FIG. 9B); CD86, TGFB1, MRC1, TGFBR2, FES, CD163, HLA, IFI44L, FES, LILRBB2, FGL2, TNFSF8, DBPB1, SIGLEC1, HLA-DPA1, MS4A6A (see e.g., FIG. 9B); MMP1, MMP12, AQP9, NCF2, TIMP1, MARCO, CD74, TGM2, TGFB1, LGALS3, CD86, TGFB1, MRC1, TGFBR2, FES, CD163, HLA, IFI44L, FES, LILRBB2, FGL2, TNFSF8, DBPB1, SIGLEC1, HLA-DPA1, MS4A6A (see e.g., FIG. 9B); TFF1, CCL20, HNF4A, AS1, BTNL8, LYZ, GATA6, ANXA10, CTSE, CYP3A5, or any combination thereof (see e.g., FIG. 9C); ISG15, OAS2, GOS2, TP63, WNT7B, IL1ORB, LY6E, IFI6, MT2A, SOD2, IFNAR2, COMMD7, IFIT3, IFIT1, SCO2, ZBP1, IL34, IFIT2, MUC16, or any combination thereof (see e.g., FIG. 9C); TFF1, CCL20, HNF4A, AS1, BTNL8, LYZ, GATA6, ANXA10, CTSE, CYP3A5, ISG15, OAS2, GOS2, TP63, WNT7B, IL1ORB, LY6E, IFI6, MT2A, SOD2, IFNAR2, COMMD7, IFIT3, IFIT1, SCO2, ZBP1, IL34, IFIT2, MUC16, or any combination thereof (see e.g., FIG. 9C); BTNL8, GATA6, CTSE, ANXA10, CDH17, MUC12, CYP3A5, LYZ, CLRN3, ST6GALNAC1, REG4, MUC1, MUC5AC, MUC17, or any combination thereof (see e.g., FIG. 9D); COL1A1, COL3A1, KRT17, COL1A2, FN1, HMGA2, LGALS1, MUC116, WNT5B, COL6A, TNC, or any combination thereof (see e.g., FIG. 9D); BTNL8, GATA6, CTSE, ANXA10, CDH17, MUC12, CYP3A5, LYZ, CLRN3, ST6GALNAC1, REG4, MUC1, MUC5AC, MUC17, COL1A1, COL3A1, KRT17, COL1A2, FN1, HMGA2, LGALS1, MUC116, WNT5B, COL6A, TNC, or any combination thereof (see e.g., FIG. 9D); TGFB3, TGFBI, TGFB1, TGFB2, FAP, WNT5A, or any combination thereof (see e.g., FIG. 15A); CDKN1A, NTRK3, MYH11, TMOD1, CLU, NR4A3, BCL2, RYR3, RANBP3L, CSPG4, CHRM3, RBM20, CXCL12, REEP1, CHRM2, ALDOA, P2RX1, LDB3, SPEG, KCNAB1, AKAP6, DES, ACTG2, MYOCD, SYNM, SOD2, JPH2, or any combination thereof (see e.g., FIG. 15A); TGFB3, TGFBI, TGFB1, TGFB2, FAP, WNT5A, CDKN1A, NTRK3, MYH11, TMOD1, CLU, NR4A3, BCL2, RYR3, RANBP3L, CSPG4, CHRM3, RBM20, CXCL12, REEP1, CHRM2, ALDOA, P2RX1, LDB3, SPEG, KCNAB1, AKAP6, DES, ACTG2, MYOCD, SYNM, SOD2, JPH2, or any combination thereof (see e.g., FIG. 15A); or any combination thereof.

In certain example embodiments, the program and/or signature can include one or more of the following cell types and/ior groups thereof: Schwann, endocrine, malignant, atypical ductal, ductal, acinar, fibroblast, smooth muscle, endothelial, nascent endothelial, or any combination thereof (See e.g., FIG. 1C); B, plasma, NK, CD4+T, CD8+T, Regulatory T, pDC, cDC1, cDC2, mregDC, macrophage, mast, or any combination thereof (see e.g., FIG. 1C); alpha, beta, delta, gamma (see e.g., FIG. 1C); acinar, acinar-REG+, or both (see e.g., FIG. 1C); Immune, malignant/ductal, fibroblast, endothelial, or any combination thereof (see e.g., FIG. 1D); immune, schwann, endocrine, malignant, atypical ductal, ductal, acinar, fibroblast, smooth muscle, endothelial, nascent endothelial, or any combination thereof (see e.g., FIGS. 6A and 8A-8B); or any combination thereof.

In certain example embodiments, the PDAC tumor program and/or signature includes or is any one or more of the following: GO homeostatic process, GO detoxification, Reactome interferon signaling, Browne interferon responsive genes, Reacctome interferon alpha beta signaling, Hallmark interferon gamma response, GO response to type I interferon, Einav interferon signature in cancer, Hecker IFNB1 targets (See e.g., FIG. 2C); contractility/neuromuscular program, mesodermal development program, Altered differentiation program, M1 program, M2 program, M0 program, or any combination thereof (FIG. 2D); Mesodermal progenitor, secretory, neurotropic, myofibroblast, or any combination thereof (See e.g., FIG. 4D); Hypoxic, interferon signaling, TNF NFKB signaling, mesenchymal matrisomal, classical activated, classical progenitor, squamous, mesenchymal cytoskeletal, cycling, or any combination thereof (see e.g. FIG. 4D); Cycling, Hypoxic, squamous, IFN signaling, Classical progenitor, classical activated, TNF-NFkB signaling, Mesenchymal cytoskeleton, mesenchymal matrisomal, or any combination thereof (See e.g., FIG. 12); GO smooth muscle contraction, GO circulatory system development, GO heart development, GO muscle structure development, Hallmark myogenesis, GO contractile fiber, GO muscle contraction, GO muscle system process, or any combination thereof (see FIG. 15A); myofibroblast, secretory, neurotropic, mesodermal progenitor, neuromuscular, and combinations thereof (FIG. 15B); Neurotropic, mesodermal progenitor, myofibroblast, secretory, or any combination thereof, (see e.g., FIG. 15C); myofibroblast, secretory, neuromuscular, neurotropic (see e.g., FIG. 15C); neurotropic, mesodermal progenitor, secretory, myofibroblast, or any combination thereof (FIG. 15D); myofibroblast, secretory, neurotropic, neuromuscular (see e.g., FIG. 15D); and non-specific immune, macrophage-enriched, macrophage-depleted A, macrophage-depleted B (see e.g., FIG. 16C).

In certain example embodiments, there is a greater likelihood of longer survival when a predominant mesenchymal matrisomal and/or a classical progenitor malignant program is detected as compared to detection of a primary classical activated, a squamous, or a mesenchymal cytoskeletal program.

In certain example embodiments, there is a greater likelihood of longer survival when a CAF secretory or neurotropic program is detected as compared to detection of a myofibroblast or mesodermal progenitor program is detected.

In certain example embodiments, a subject having a classical-like malignant expression program has the greatest likelihood of time to progression and longest survival.

In certain example embodiments, a subject having an immunomodulatory CAF expression program has the greatest likelihood of time to progression.

In certain example embodiments, a subject having a neuronal like malignant expression program or a malignant squamoid expression program has the greatest likelihood of least time to progression.

In certain example embodiments, a subject having an adhesive CAF expression program has the greatest likelihood of shortest survival.

In certain example embodiments, the PDAC patient had or is concurrently receiving a neoadjuvant therapy.

In certain example embodiments, detecting comprises a single cell RNA sequencing technique.

In certain example embodiments, detecting comprises a single-nucleus RNA sequencing technique.

In certain example embodiments, the single-nucleus RNA sequencing technique is optimized for pancreatic tissue.

In certain example embodiments, the single-nucleus RNA sequencing technique is optimized for frozen samples.

In certain example embodiments, the single-nucleus RNA sequencing technique comprises screening a sample for an RNA integrity number and performing single nucleus RNA sequencing only on samples with an RNA integrity number of 6 or more.

In certain example embodiments, detecting comprises a spatially-resolved transcriptomics technique.

The signature as defined herein (being it a gene signature, protein signature or other genetic or epigenetic signature) can be used to indicate the presence of a cell type, a subtype of the cell type, the state of the microenvironment of a population of cells, a particular cell type population or subpopulation, and/or the overall status of the entire cell (sub)population. Furthermore, the signature may be indicative of cells within a population of cells in vivo. The signature may also be used to suggest for instance particular therapies, or to follow up treatment, or to suggest ways to modulate immune systems. The signatures of the present invention may be discovered by analysis of expression profiles of single-cells within a population of cells from isolated samples (e.g., Sys tumor samples), thus allowing the discovery of novel cell subtypes or cell states that were previously invisible or unrecognized. The presence of subtypes or cell states may be determined by subtype specific or cell state specific signatures. The presence of these specific cell (sub)types or cell states may be determined by applying the signature genes to bulk sequencing data in a sample. In certain embodiments, the signatures of the present invention may be microenvironment specific, such as their expression in a particular spatio-temporal context. In certain embodiments, signatures as discussed herein are specific to a particular pathological context. In certain embodiments, a combination of cell subtypes having a particular signature may indicate an outcome. In certain embodiments, the signatures can be used to deconvolute the network of cells present in a particular pathological condition. In certain embodiments, the presence of specific cells and cell subtypes are indicative of a particular response to treatment, such as including increased or decreased susceptibility to treatment. The signature may indicate the presence of one particular cell type. In one embodiment, the novel signatures are used to detect multiple cell states or hierarchies that occur in subpopulations of cells that are linked to particular pathological condition (e.g., inflammation), or linked to a particular outcome or progression of the disease, or linked to a particular response to treatment of the disease.

The invention provides biomarkers (e.g., phenotype specific or cell type) for the identification, diagnosis, prognosis and manipulation of cell properties, for use in a variety of diagnostic and/or therapeutic indications. Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from an immune response based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

[The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-á-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-á-vis a control subject or subject population.

In some embodiments, an altered quality, quantity, and/or phenotype of PDAC tumour cells in or from the subject compared to a suitable control or reference value(s) can indicate that the subject would benefit from or is in need of a specific treatment. In some of such embodiments, the method can further include administration of such a specifically identified treatments.

In some embodiments, an altered quality, quantity, and/or phenotype of PDAC tumour cells in or from the subject compared to a suitable control or reference value(s) can indicate that the subject falls into a particular group or subset of patients all diagnosed with or having the same general disease (e.g. cancer, pancreatic cancer, PDAC, etc.), where each group optionally can be treated in different ways specific to each group to improve outcome, as well as, improve general patient care by allowing greater precision prediction of individual patient survival and/or treatment response.

The methods described herein can rely on comparing the quantity or quality of PDCA tumour cell population cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), any gene or transcript sequencing method, including but not limited to, RNA-seq, single cell RNA-seq, single nucleus RNAseq, spatial transcriptomics, spatial proteomics, quantitative RT-PCR, single cell qPCR, FISH, RNA-FISH, MERFISH (multiplex (in situ) RNA FISH), in situ hybridization, CRISPR-effector system mediated screening assay (e.g., SHERLOCK assay), compressed sensing, and any combination thereof. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March;26(3):317-25).

MS methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g., diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Single Cell RNA Sequencing

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516⁻⁵³⁵, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January;12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October;14(10):955-958; International patent application number PCT/US2016/059239, published as WO2017164936 on Sep. 28, 2017; International patent application number PCT/US2018/060860, published as WO/2019/094984 on May 16, 2019; International patent application number PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety. In some embodiments the snRNA-seq method is optimized for a pancreatic sample. In some embodiments the snRNA-seq method is optimized for a frozen sample. In some embodiments, the snRNA-seq is optimized for a frozen pancreatic sample. In some embodiments, the snRNA-seq method comprises determining an RNA integrity number of a sample. In some embodiments, the snRNA-seq method comprises using only samples with an RNA integrity number of 6 or greater or greater than 6. Additional details can be found in the Working Examples elsewhere herein.

In certain embodiments, the invention involves the Assay for Transposase Accessible Chromatin using sequencing (ATAC-seq) as described. See e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22;348(6237):910-4. doi: 10.1126/science.aabl601. Epub 2015 May 7; US20160208323A1; US20160060691A1; and WO2017156336A1).

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854,5,288,644, 5,324,633, 5,432,049, 5,470,710, 5,492,806, 5,503,980, 5,510,270, 5,525,464, 5,547,839, 5,580,732, 5,661,028, 5,800,992, the disclosures of which are incorporated herein by reference, as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-Interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization with Nucleic Acid Probes”, Elsevier Science Publishers B.V_{HH}. (1993) and Kricka, “Nonisotopic DNA Probe Techniques”, Academic Press, San Diego, Calif (1992).

### Compressed Sensing

Mammalian genomes contain approximately 20,000 genes, and mammalian expression profiles are frequently studied as vectors with 20,000 entries corresponding to the abundance of each gene. It is often assumed that studying gene expression profiles requires measuring and analyzing these 20,000 dimensional vectors, but some mathematical results show that it is often possible to study high-dimensional data in low dimensional space without losing much of the pertinent information. In one embodiment of the present invention, less than 20,000 aptamers are used to detect protein expression in single cells. Not being bound by a theory, working in low dimensional space offers several advantages with respect to computation, data acquisition and fundamental insights about biological systems.

In one embodiment, aptamers are chosen for protein targets that are generally part of gene modules or programs, whereby detection of a protein allows for the ability to infer expression of other proteins present in a module or gene program. Samples are directly compared based only on the measurements of these signature genes.

In alternative embodiments, sparse coding or compressed sensing methods can be used to infer large amounts of data with a limited set of target proteins. Not being bound by a theory, the abundance of each of the 20,000 genes can be recovered from random composite measurements. In this regard, reference is made to Cleary et al., “Composite measurements and molecular compressed sensing for highly efficient transcriptomics” posted on Jan. 2, 2017 at biorxiv.org/content/early/2017/01/02/091926, doi.org/10.1101/091926, incorporated herein by reference in its entirety.

In some embodiments, the method of diagnosing, prognosing, and/or monitoring, can include obtaining a sample, such as a PDCA tumor sample, and analyzing cell signatures from cells in bulk or individually by one or more methods described herein. In some embodiments, the method includes analyzing PDCA tumor sample using snRNA-seq and/or spatial transcriptomics. In some embodiments, the tumor sample is obtained before resection, such as by biopsy. In some embodiments, the tumor sample is obtained after tumor resection.

In some embodiments, for example, a tissue sample may be obtained and analyzed for specific cell markers (IHC) or specific transcripts (e.g., RNA-FISH). Tissue samples for diagnosis, prognosis or detecting may be obtained by endoscopy. In one embodiment, a sample may be obtained by endoscopy and analyzed by FACS. As used herein, “endoscopy” refers to a procedure that uses an endoscope to examine the interior of a hollow organ or cavity of the body. The endoscope may include a camera and a light source. The endoscope may include tools for dissection or for obtaining a biological sample. A cutting tool can be attached to the end of the endoscope, and the apparatus can then be used to perform surgery. Applications of endoscopy that can be used with the present invention include, but are not limited to examination of the esophagus, stomach and duodenum (esophagogastroduodenoscopy); small intestine (enteroscopy); large intestine/colon (colonoscopy, sigmoidoscopy); bile duct; rectum (rectoscopy) and anus (anoscopy), both also referred to as (proctoscopy); respiratory tract; nose (rhinoscopy); lower respiratory tract (bronchoscopy); ear (otoscope); urinary tract (cystoscopy); female reproductive system (gynoscopy); cervix (colposcopy); uterus (hysteroscopy); fallopian tubes (falloposcopy); normally closed body cavities (through a small incision); abdominal or pelvic cavity (laparoscopy); interior of a joint (arthroscopy); or organs of the chest (thoracoscopy and mediastinoscopy).

### Treating PDAC

Described in certain example embodiments herein are methods treating pancreatic ductal adenocarcinoma (PDAC) in a subject in need thereof comprising: preventing a shift in the state of a malignant cell from a classical progenitor state to a basal-like state or a terminally-differentiated state; modulating a cell state of a malignant cell from a basal-like state or a terminally-differentiated state to a classical progenitor state; inhibiting, preventing, or modulating expression of a neuronal like expression program in a malignant cells; inhibiting, preventing expression or modulating expression of a malignant squamoid expression program in a malignant cell, inhibiting, preventing, or modulating expression of an adhesive CAF expression program in a CAF cell; or any combination thereof.

In certain example embodiments, the subject has had neoadjuvant therapy; is concurrently receiving or undergoing neoadjuvant therapy; or the subject has not had neoadjuvant therapy.

In some embodiments, a malignant cell state is characterized by a malignant cell signature comprising: a lineage specific expression program selected from a squamous program, a mesenchymal cytoskeletal program, mesenchymal matrisomal program, a classical progenitor program, or a classical activated program; a lineage specific expression program selected from a squamous program, a mesenchymal program, an induced basal-like program, a classical progenitor program, a classical acinar-like program, and a classical neuroendocrine-like program; a cell state specific expression program selected from a cycling program, a hypoxic program, TNF-NFkB signaling program, or an interferon signaling program; a cell state specific expression program selected from a cycling program, a TNF-NFkB signaling program, or an interferon signaling program; a neoadjuvant treated malignant cell expression program; an untreated malignant cell expression program; a basal-like malignant cell expression program; a classic-like malignant cell expression program; an immune microniche signature; or a combination thereof.

In certain example embodiments, the malignant cell signature and/or program comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

In certain example embodiments, modulating the cell state comprises reducing the distance in gene expression space between the basal-like malignant cell state and the classic-like malignant cell states.

In certain example embodiments, wherein the gene expression spaces comprises 10 or more genes, 20 or more genes, 30 or more genes, 40 or more genes, 50 or more genes, 100 or more genes, 500 or more genes, or 1000 or more genes.

In certain example embodiments, the distance is measured by a Euclidean distance, Pearson coefficient, Spearman coefficient, or combination thereof.

In certain example embodiments, modulation comprises increasing or decreasing expression of one or more genes, gene expression cassettes, or gene expression signatures.

In certain example embodiments, modulating or preventing comprises administering a modulating agent to the subject.

In certain example embodiments, the modulating agent comprises a therapeutic antibody or fragment thereof, antibody-like protein scaffold, aptamer, polypeptide, a polynucleotide, a genetic modifying agent or system, a small molecule therapeutic, a chemotherapeutic, small molecule degrader, inhibitor, an immunomodulator, or a combination thereof.

Described in certain example embodiments herein are methods, of treating a subject having PDAC, the method comprising: administering a neoadjuvant therapy to the subject; and administering a PDAC malignant cell modulating agent to the subject, administering an immune modulator to the subject, administering a CAF modulating agent to the subject, or any combination thereof to the subject.

Described in certain example embodiments herein are methods treating a subject having PDAC, the method comprising: detecting, in one or more PDAC tumor cells, a malignant cell signature, program, or both; a cancer-associated fibroblast (CAF) signature, program, or both; an immune microniche signature, program, or both; a tumor spatial neighborhood, one or more co-expressed receptor-ligand pairs, or any combination thereof, and administering or applying a PDAC treatment to the subject in need thereof, wherein the treatment is optionally a tumor resection, a chemotherapy, a radiation therapy, a neoadjuvant, a malignant cell signature and/or program modulating agent, a BCL-2 inhibitor, a tyrosine kinase inhibitor, a TGFbeta modulator, a myeloid cell agonist, a CXCR4 inhibitor, a HER2 inhibitor, or any combination thereof.

In certain example embodiments, the malignant cell signature and/or program comprises a lineage specific expression program selected from: a squamous program, a mesenchymal cytoskeletal program, mesenchymal matrisomal program; a classical progenitor program, a classical activated program, or any combination thereof lineage specific expression program selected from: a squamous program, a mesenchymal program, an induced basal-like program, a classical progenitor program, a classical acinar-like program, a classical neuroendocrine-like program, or any combination thereof; a cell state specific expression program selected from: a cycling program, a hypoxic program, TNF-NFkB signaling program, an interferon signaling program, or any combination thereof, a cell state specific expression program selected from: a cycling program, a TNF-NFkB signaling program, or an interferon signaling program, or any combination thereof, a neoadjuvant treated malignant cell expression program; an untreated malignant cell expression program; a cell state expression program selected from: a neuronal-like program, a neuroendocrine like program, a mesenchymal program, a squamoid program, a MYC signaling program, a cycling (G2M) program, a cycling (S) program, or any combination thereof; a lineage specific expression program selected from: an acinar-like program, a classical-like program, a basaloid program, a squamoid program, a mesenchymal program, a neuroendocrine like program, a neuronal like program, or any combination thereof, or any combination thereof.

In certain example embodiments, the CAF signature and/or program comprises a myofibroblast program; a neurotropic program; a secretory program; a mesodermal progenitor program a neuromuscular program; or any combination thereof, comprises a neoadjuvant treated CAF signature and/or program selected from: a neuromuscular program, a secretory program, a neurotropic program, or any combination thereof, comprises an untreated CAF signature and/or program selected from: a mesodermal progenitor program, a myofibroblast program, a neurotropic program, a secretory program, or any combination thereof, or comprises an adhesive expression program, an immunomodulatory expression program, a myofibroblastic progenitor expression program, or a neurotropic expression program.

In certain example embodiments, the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39, and Table 7, or any combination thereof.

In certain example embodiments, the method comprises, detecting, in one or more a PDAC tumor cells, an untreated tumor malignant cell signature and/or program and an untreated CAF signature and/or program, wherein the untreated tumor malignant cell signature and/or program comprises a lineage specific expression program selected from: a squamous program, a mesenchymal cytoskeletal program, mesenchymal matrisomal program, a classical progenitor program, a classical activated program, or any combination thereof, and the untreated tumor CAF signature and/or program is selected from: a mesodermal progenitor program, a myofibroblast program, a neurotropic program, a secretory program, or any combination thereof.

In certain example embodiments, the method further comprises determining a tumor heterogeneity score for the PDAC tumor, wherein the tumor heterogeneity score is calculated by determining a number of highly expressed programs in the one or more PDAC cells.

In certain example embodiments, the method further comprises assigning the PDAC tumor to a single malignant class and to a single CAF class, wherein the malignant class is selected from A0, A1, A2, S0, S1, S2, C0, C1, C2, M0, M1, M2, P0, P1, or P2, and wherein the CAF class is selected from S0, S1, NO, N1, M0, M1, P0, or P1.

In certain example embodiments, the PDAC tumor is assigned to a combined risk class that integrates the malignant risk group and CAF risk group class and is selected from: a low combined risk group, a low-intermediate combined risk group, a high-intermediate risk group, or a high combined risk group, wherein a PDAC tumor in a low malignant risk group and in a low CAF risk group is classified into the low combined risk group; a PDAC tumor in a high malignant risk and in a high CAF risk is classified into the high combined risk group; a PDAC tumor in an intermediate malignant risk group or in an intermediate CAF risk and in a high malignant risk or in a high CAF risk is classified into the high-intermediate combined risk group; and a PDAC tumor in a low malignant risk group and in a high CAF risk group, a PDAC tumor in a high malignant risk group and in a low CAF risk group, a PDAC tumor in a low malignant risk group and in a low CAF risk group, a PDAC tumor in an intermediate malignant risk group and in an intermediate CAF risk group, a PDAC tumor in a low malignant risk group and in an intermediate CAF risk group is classified into the low-intermediate combined risk group.

In certain example embodiments, a subject with a PDAC tumor in low combined risk group has the greatest likelihood of longest survival.

In certain example embodiments, a subject having a classical-like malignant expression program has the greatest likelihood of time to progression and longest survival.

In certain example embodiments, a subject having an immunomodulatory CAF expression program has the greatest likelihood of time to progression.

In certain example embodiments, a subject having a neuronal like malignant expression program or a malignant squamoid expression program has the greatest likelihood of least time to progression.

In certain example embodiments, a subject having an adhesive CAF expression program has the greatest likelihood of shortest survival.

In certain example embodiments, the tumor spatial neighborhood is a treatment enriched neighborhood, a squamoid-basaloid neighborhood, or a classical neighborhood.

In certain example embodiments, the one or more co-expressed receptor-ligand pairs is selected from an Epithelial compartment—CAF compartment pair; an Epithelial compartment—Immune compartment; a CAF compartment and Immune compartment pair; or any combination thereof.

In certain example embodiments, the malignant cell signature comprises one or more biomarkers, expression programs, biologic programs, receptor-ligand interactions, cell state distribution, cell type distribution, or any combination thereof as in any of 1B-1D, 2A-2D, 3A- 3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

In certain example embodiments, the CAF cell signature one or more biomarkers, expression programs, biologic programs, receptor-ligand interactions, cell state distribution, cell type distribution, or any combination thereof FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

In certain example embodiments, the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39, and Table 7, or any combination thereof.

In certain example embodiments, the method comprises, detecting, in one or more a PDAC tumor cells, an untreated tumor malignant cell signature and/or program and an untreated CAF signature and/or program, wherein the untreated tumor malignant cell signature and/or program comprises a lineage specific expression program selected from: a squamous program, a mesenchymal cytoskeletal program, mesenchymal matrisomal program, a classical progenitor program, a classical activated program, or any combination thereof; and the untreated tumor CAF signature and/or program is selected from: a mesodermal progenitor program, a myofibroblast program, a neurotropic program, a secretory program, or any combination thereof.

In certain example embodiments, the PDAC treatment is a neoadjuvant therapy.

In certain example embodiments, the PDAC treatment comprises preventing a shift in the state of a malignant cell from a classical progenitor state to a basal-like state or a terminally-differentiated state; modulating a cell state of a malignant cell from a basal-like state or a terminally-differentiated state to a classical progenitor state; inhibiting, preventing, or modulating expression of a neuronal like expression program in a malignant cells; inhibiting, preventing expression or modulating expression of a malignant squamoid expression program in a malignant cell, inhibiting, preventing, or modulating expression of an adhesive CAF expression program in a CAF cell; or any combination thereof.

In certain example embodiments, the PDAC treatment is a PDAC signature modulating agent.

In certain example embodiments, the PDAC modulating agent is selected by performing a PDAC modulating agent screening method as described elsewhere herein.

In certain example embodiments, wherein the subject has had neoadjuvant therapy; is concurrently receiving or undergoing neoadjuvant therapy; or the subject has not had neoadjuvant therapy.

In certain example embodiments, the subject has had a PDAC tumor resected prior to administration.

In certain example embodiments, the subject has not had a PDAC tumor resected prior to administration.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse). In certain embodiments, the present invention provides for one or more therapeutic agents against combinations of targets identified. Targeting the identified combinations may provide for enhanced or otherwise previously unknown activity in the treatment of disease.

### Adoptive Cell Transfer

In some embodiments, a method of treatment can include treatment with adoptive cell transfer.

As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June;24(6):724-730; Besser et al., (2010) Clin. Cancer Res 16 (9) 2646⁻⁵⁵; Dudley et al., (2002) Science 298 (5594): 850-4; and Dudley et al., (2005) Journal of Clinical Oncology 23 (10): 2346⁻⁵⁷.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); κ-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGa1p(1⁻⁴)bDG1cp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGa1p(1⁻⁴)bDG1cp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR5IE2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-K1-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein);, fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor ber-ab1 (protein of 190KD ber-ab1); Pm1/RARa (promyelocytic leukaemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of. CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and R chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CD5, CD9, CD 16, CD22, CD33, CD37, CD64, CD80, CD86, CD134, CD137, CD154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; and 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; and 9,102,761). Third-generation CARs include a combination of costimulatory endodomains, such a CD3ζ-chain, CD97, GDI 1a-CD18, CD2, ICOS, CD27, CD154, CD5, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO 2014/134165; PCT Publication No. WO 2012/079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3(or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of 4-1BB, CD27, and CD28. In certain embodiments, a chimeric antigen receptor may have the design as described in U.S. Pat. No. 7,446,190, comprising an intracellular domain of CD3(chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS)) (SEQ ID NO:1). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3(chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-(molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-(molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 2) and continuing all the way to the carboxy-terminus of the protein. The sequence is reproduced herein: IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS (SEQ ID NO: 1). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly, Example 1 and Table 1 of WO 2015/187528, incorporated by reference herein, demonstrate the generation of anti-CD19 CARs based on a fully human anti-CD19 monoclonal antibody (47G4, as described in US Patent Publication No. 2010/0104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcBRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO 2015/187528 and an intracellular T-cell signaling domain as set forth in Table 1 of WO 2015/187528. Preferably, the antigen is CD19, more preferably the antigen-binding element is an anti-CD19 scFv, even more preferably the mouse or human anti-CD19 scFv as described in Example 1 of WO 2015/187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO 2015/187528.

By means of an example and without limitation, chimeric antigen receptor that recognizes the CD70 antigen is described in International Patent Publication No. WO 2012/058460A2 (see also, Park et al., CD70 as a target for chimeric antigen receptor T cells in head and neck squamous cell carcinoma, Oral Oncol. 2018 March;78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am.J.Pathol. 1995;147:1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005;65:5428⁻⁵⁴³⁸) Physiologically, CD70 expression is transient and restricted to a subset of highly activated T, B, and dendritic cells.

By means of an example and without limitation, chimeric antigen receptor that recognizes BCMA has been described (see, e.g., US Patent Publication No. 2016/0046724 A1; International Patent Publication Nos. WO 2016/014789 A2, WO 2017/211900 A1, WO 2015/158671 A1, WO2018028647A1, and WO 2013/154760 A1; and US Patent Publication Nos. 2018/0085444 A1 and 2017/0283504 A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., International Patent Publication Nos. WO 2013/044225, WO 2016/000304, WO 2015/057834, WO 2015/057852, WO 2016/070061, U.S. Pat. No. 9,233,125, and US Patent Publication No. 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3(and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs CS, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e., intracavity delivery) or directly into a tumor prior to resection (i.e., intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount (e.g., number) of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be done directly by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 2013/0071414; PCT Patent Publication Nos. WO 2011/146862, WO 2014/011987, WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1;23(9):2255-2266. doi: 10.1158/1078-0432.CCR-16-1300. Epub 2016 November 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHIC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and p, which assemble to form a heterodimer and associates with the CD3-transducing subunits to form the T cell receptor complex present on the cell surface. Each a and p chain of the TCR consists of an immunoglobulin-like N-terminal variable (V) and constant (C) region, a hydrophobic transmembrane domain, and a short cytoplasmic region. As for immunoglobulin molecules, the variable region of the α and p chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1;112(12):4746⁻⁵⁴). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL1ORB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SUP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD- L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, J-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, 0-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled 02-microglobulin (02m) into MHC class I/02m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™ BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Modulating PDAC Signatures

In some embodiments, the method, such a method of treatment, includes modulating a PDAC signature, or, maintaining (i.e., preventing a shift in signature away from a desired signature) a desired PDAC signature. In general, such methods include administering a modulating agent to a subject.

### Modulating Agents

As used herein, “modulating” or “to modulate” generally means either reducing or inhibiting the expression or activity of, or alternatively increasing the expression or activity of a target or antigen. In particular, “modulating” or “to modulate” can mean either reducing or inhibiting the activity of, or alternatively increasing a (relevant or intended) biological activity of, a target or antigen as measured using a suitable in vitro, cellular or in vivo assay (which will usually depend on the target involved), by at least 5%, at least 10%, at least 25%, at least 50%, at least 60%, at least 70%, at least 80%, at least 90%, or more, compared to activity of the target in the same assay under the same conditions but without the presence of an agent. An “increase” or “decrease” refers to a statistically significant increase or decrease respectively. For the avoidance of doubt, an increase or decrease will be at least 10% relative to a reference, such as at least 10%, at least 20%, at least 30%, at least 40%, at least 50%, a t least 60%, at least 70%, at least 80%, at least 90%, at least 95%, at least 97%, at least 98%, or more, up to and including at least 100% or more, in the case of an increase, for example, at least 2-fold, at least 3-fold, at least 4-fold, at least 5-fold, at least 6-fold, at least 7-fold, at least 8-fold, at least 9-fold, at least 10-fold, at least 50-fold, at least 100-fold, or more. “Modulating” can also involve effecting a change (which can either be an increase or a decrease) in affinity, avidity, specificity and/or selectivity of a target or antigen. “Modulating” can also mean effecting a change with respect to one or more biological or physiological mechanisms, effects, responses, functions, pathways or activities in which the target or antigen (or in which its substrate(s), ligand(s) or pathway(s) are involved, such as its signaling pathway or metabolic pathway and their associated biological or physiological effects) is involved. Again, as will be clear to the skilled person, such an action as an agonist or an antagonist can be determined in any suitable manner and/or using any suitable assay known or described herein (e.g., in vitro or cellular assay), depending on the target or antigen involved.

Modulating can, for example, also involve allosteric modulation of the target and/or reducing or inhibiting the binding of the target to one of its substrates or ligands and/or competing with a natural ligand, substrate for binding to the target. Modulating can also involve activating the target or the mechanism or pathway in which it is involved. Modulating can for example also involve effecting a change in respect of the folding or confirmation of the target, or in respect of the ability of the target to fold, to change its conformation (for example, upon binding of a ligand), to associate with other (sub)units, or to disassociate. Modulating can for example also involve effecting a change in the ability of the target to signal, phosphorylate, dephosphorylate, and the like.

As used herein, an “agent” can refer to a protein-binding agent that permits modulation of activity of proteins or disrupts interactions of proteins and other biomolecules, such as but not limited to disrupting protein-protein interaction, ligand-receptor interaction, or protein-nucleic acid interaction. Agents can also refer to DNA targeting or RNA targeting agents. Agents can also refer to a protein,. Agents may include a fragment, derivative and analog of an active agent. The terms “fragment,” “derivative” and “analog” when referring to polypeptides as used herein refers to polypeptides which either retain substantially the same biological function or activity as such polypeptides. An analog includes a proprotein which can be activated by cleavage of the proprotein portion to produce an active mature polypeptide. Such agents include, but are not limited to, antibodies (“antibodies” includes antigen-binding portions of antibodies such as epitope- or antigen-binding peptides, paratopes, functional CDRs; recombinant antibodies; chimeric antibodies; humanized antibodies; nanobodies; tribodies; midibodies; or antigen-binding derivatives, analogs, variants, portions, or fragments thereof), protein-binding agents, nucleic acid molecules, small molecules, recombinant protein, peptides, aptamers, avimers and protein-binding derivatives, portions or fragments thereof. An “agent” as used herein, may also refer to an agent that inhibits expression of a gene, such as but not limited to a DNA targeting agent (e.g., CRISPR system, TALE, Zinc finger protein) or RNA targeting agent (e.g., inhibitory nucleic acid molecules such as RNAi, miRNA, ribozyme).

As used in the context of shifting or modulating a PDAC signature herein, “modulating” also includes maintaining an initial signature (i.e. preventing a shift in signature. As used in the context of shifting or modulating a PDAC signature herein, “modulating agent” includes agents capable of causing a shift in a PDAC signature from an initial signature indicative of a first cell or population state or type to a second signature indicative of a second cell or population state or type, as well as agents capable of maintaining an initial signature. In some embodiments, it may be advantageous to maintain an initial signature, particularly in the context of preventing a shift to a signature that is associated with a less desirable cell or population state or type. As used in this context herein, “modulating agent” is inclusive of pharmaceutical agents (e.g. small molecule compounds, biologics, and the like) that can be administered in a dosage form to a subject as well as physical treatments such as surgical resection, radiation, thermal treatments, and the like that can be applied to a subject and not necessarily in a dosage form. In some embodiments, a modulating agent is administered to a subject before, during, and/or after neoadjuvant treatment and/or PDAC tumor resection.

The agents of the present invention may be modified, such that they acquire advantageous properties for therapeutic use (e.g., stability and specificity), but maintain their biological activity.

It is well known that the properties of certain proteins can be modulated by attachment of polyethylene glycol (PEG) polymers, which increases the hydrodynamic volume of the protein and thereby slows its clearance by kidney filtration. (See, e.g., Clark et al., J. Biol. Chem. 271: 21969-21977 (1996)). Therefore, it is envisioned that certain agents can be PEGylated (e.g., on peptide residues) to provide enhanced therapeutic benefits such as, for example, increased efficacy by extending half-life in vivo. In certain embodiments, PEGylation of the agents may be used to extend the serum half-life of the agents and allow for particular agents to be capable of crossing the blood-brain barrier. Thus, in one embodiment, PEGylating inhibitor of HDAC and/or CDK4/6 improve the pharmacokinetics and pharmacodynamics of the inhibitors.

In regard to peptide PEGylation methods, reference is made to Lu et al., Int. J. Pept. Protein Res.43: 127-38 (1994); Lu et al., Pept. Res. 6: 140-6 (1993); Felix et al., Int. J. Pept. Protein Res. 46: 253-64 (1995); Gaertner et al., Bioconjug. Chem. 7: 38-44 (1996); Tsutsumi et al., Thromb. Haemost. 77: 168-73 (1997); Francis et al., hit. J. Hematol. 68: 1-18 (1998); Roberts et al., J. Pharm. Sci. 87: 1440-45 (1998); and Tan et al., Protein Expr. Purif 12:45⁻⁵² (1998). Polyethylene glycol or PEG is meant to encompass any of the forms of PEG that have been used to derivatize other proteins, including, but not limited to, mono-(C₁₋₁₀) alkoxy or aryloxy-polyethylene glycol. Suitable PEG moieties include, for example, 40 kDa methoxy poly(ethylene glycol) propionaldehyde (Dow, Midland, Mich.); 60 kDa methoxy poly(ethylene glycol) propionaldehyde (Dow, Midland, Mich.); 40 kDa methoxy poly(ethylene glycol) maleimido-propionamide (Dow, Midland, Mich.); 31 kDa alpha-methyl-w-(3-oxopropoxy), polyoxyethylene (NOF Corporation, Tokyo); mPEG2-NHS-40k (Nektar); mPEG2-MAL-40k (Nektar), SUNBRIGHT GL2-400MA ((PEG)240 kDa) (NOF Corporation, Tokyo), SUNBRIGHT ME-200MA (PEG20 kDa) (NOF Corporation, Tokyo). The PEG groups are generally attached to the peptide via acylation or alkylation through a reactive group on the PEG moiety (for example, a maleimide, an aldehyde, amino, thiol, or ester group) to a reactive group on the peptide (for example, an aldehyde, amino, thiol, a maleimide, or ester group).

The PEG molecule(s) may be covalently attached to any Lys, Cys, or K(CO(CH₂)₂SH) residues at any position in a peptide. In certain embodiments, the peptides described herein can be PEGylated directly to any amino acid at the N-terminus by way of the N-terminal amino group. A “linker arm” may be added to a peptide to facilitate PEGylation. PEGylation at the thiol side-chain of cysteine has been widely reported (see, e.g., Caliceti & Veronese, Adv. Drug Deliv. Rev. 55: 1261-77 (2003)). If there is no cysteine residue in the peptide, a cysteine residue can be introduced through substitution or by adding a cysteine to the N-terminal amino acid. PEGylaeion can be affected through the side chains of a cysteine residue added to the N-terminal amino acid.

In exemplary embodiments, the PEG molecule(s) may be covalently attached to an amide group in the C-terminus of a peptide. In preferred embodiments, there is at least one PEG molecule covalently attached to the peptide. In certain embodiments, the PEG molecule used in modifying an agent of the present invention is branched while in other embodiments, the PEG molecule may be linear. In particular aspects, the PEG molecule is between 1 kDa and 100 kDa in molecular weight. In further aspects, the PEG molecule is selected from 10, 20, 30, 40, 50, 60, and 80 kDa. In further still aspects, it is selected from 20, 40, or 60 kDa. Where there are two PEG molecules covalently attached to the agent of the present invention, each is 1 to 40 kDa and in particular aspects, they have molecular weights of 20 and 20 kDa, 10 and 30 kDa, 30 and 30 kDa, 20 and 40 kDa, or 40 and 40 kDa. In particular aspects, the agent (e.g., neuromedin U receptor agonists or antagonists) contain mPEG-cysteine. The mPEG in mPEG-cysteine can have various molecular weights. The range of the molecular weight is preferably 5 kDa to 200 kDa, more preferably 5 kDa to 100 kDa, and further preferably 20 kDa to 60 kDA. The mPEG can be linear or branched.

In particular embodiments, the agents (include a protecting group covalently joined to the N-terminal amino group. In exemplary embodiments, a protecting group covalently joined to the N-terminal amino group of the agent reduces the reactivity of the amino terminus under in vivo conditions. Amino protecting groups include C₁₋₁₀ alkyl, C₁₋₁₀ substituted alkyl, C₂₋₁₀ alkenyl, C₂₋₁₀ substituted alkenyl, aryl, —C₁₋₆ alkyl aryl, C(O) (CH₂)₁₋₆—COOH, —C(O)—C₁₋₆ alkyl, C(O)-aryl, C(O)—O—C₁₋₆ alkyl, or C(O)—O-aryl. In particular embodiments, the amino terminus protecting group is selected from the group consisting of acetyl, propyl, succinyl, benzyl, benzyloxycarbonyl, and t-butyloxycarbonyl. In other embodiments, deamination of the N-terminal amino acid is another modification that may be used for reducing the reactivity of the amino terminus under in vivo conditions.

Chemically modified compositions of the agents wherein the agent is linked to a polymer are also included within the scope of the present invention. The polymer selected is usually modified to have a single reactive group, such as an active ester for acylation or an aldehyde for alkylation, so that the degree of polymerization may be controlled. Included within the scope of polymers is a mixture of polymers. Preferably, for therapeutic use of the end-product preparation, the polymer will be pharmaceutically acceptable. The polymer or mixture thereof may include but is not limited to polyethylene glycol (PEG), monomethoxy-polyethylene glycol, dextran, cellulose, or other carbohydrate based polymers, poly-(N-vinyl pyrrolidone) polyethylene glycol, propylene glycol homopolymers, a polypropylene oxide/ethylene oxide co-polymer, polyoxyethylated polyols (for example, glycerol), and polyvinyl alcohol.

In other embodiments, the agents are modified by PEGylation, cholesterylation, or palmitoylation. The modification can be to any amino acid residue. In preferred embodiments, the modification is to the N-terminal amino acid of the agent, either directly to the N-terminal amino acid or by way coupling to the thiol group of a cysteine residue added to the N-terminus or a linker added to the N-terminus such as trimesoyl tris(3,5-dibromosalicylate (Ttds). In certain embodiments, the N-terminus of the agent comprises a cysteine residue to which a protecting group is coupled to the N-terminal amino group of the cysteine residue and the cysteine thiolate group is derivatized with N-ethylmaleimide, PEG group, cholesterol group, or palmitoyl group. In other embodiments, an acetylated cysteine residue is added to the N-terminus of the agents, and the thiol group of the cysteine is derivatized with N-ethylmaleimide, PEG group, cholesterol group, or palmitoyl group. In certain embodiments, the agent of the present invention is a conjugate. In certain embodiments, the agent of the present invention is a polypeptide consisting of an amino acid sequence which is bound with a methoxypolyethylene glycol(s) via a linker.

Substitutions of amino acids may be used to modify an agent of the present invention. The phrase “substitution of amino acids” as used herein encompasses substitution of amino acids that are the result of both conservative and non-conservative substitutions. Conservative substitutions are the replacement of an amino acid residue by another similar residue in a polypeptide. Typical but not limiting conservative substitutions are the replacements, for one another, among the aliphatic amino acids Ala, Val, Leu and Ile; interchange of Ser and Thr containing hydroxy residues, interchange of the acidic residues Asp and Glu, interchange between the amide-containing residues Asn and Gln, interchange of the basic residues Lys and Arg, interchange of the aromatic residues Phe and Tyr, and interchange of the small-sized amino acids Ala, Ser, Thr, Met, and Gly. Non-conservative substitutions are the replacement, in a polypeptide, of an amino acid residue by another residue which is not biologically similar. For example, the replacement of an amino acid residue with another residue that has a substantially different charge, a substantially different hydrophobicity, or a substantially different spatial configuration.

In certain embodiments, the present invention provides for one or more therapeutic agents. In certain embodiments, the one or more agents comprises a small molecule inhibitor, small molecule degrader (e.g., PROTAC), genetic modifying agent, antibody, antibody fragment, antibody-like protein scaffold, aptamer, protein, or any combination thereof.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking a binding site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57:107-123; and Lai et al., Modular PROTAC Design for the Degradation of Oncogenic BCR-ABL Angew Chem Int Ed Engl. 2016 Jan. 11; 55(2): 807-810).

In certain embodiments, combinations of targets are modulated. In certain embodiments, an agent against one of the targets in a combination may already be known or used clinically. In certain embodiments, targeting the combination may require less of the agent as compared to the current standard of care and provide for less toxicity and improved treatment.

In certain embodiments, a method of treating PDAC comprises administering or more agents capable of modulating or maintaining (i.e., preventing a shift in) the expression, activity, or function of one or more biomarkers of a malignant signature, a CAF signature, an immune microniche signature, or a combination thereof. In certain embodiments, a method of treating PDAC comprises administering one or more agents capable of modulating or maintaining the expression, activity, or function of one or more biomarkers of a malignant signature such that the signature is shifted to a classical-like signature. In some embodiments, the method of treating PDAC comprises administering one or more agents capable of maintaining a classic-like malignant signature. Such signatures are described in greater detail elsewhere herein.

In some embodiments, the modulating agent is selected from HDAC inhibitor, a CDK4/6 inhibitor, a checkpoint inhibitor, an immunomodulator, an antibody, a genetic modulating agent, a chemotherapeutic, an antineoplastic agent, or a combination thereof.

In some embodiments, CD40 antibodies are used as a modulating agent alone or in combination with another agent or therapy such as a chemotherapy and/or PD-I inhibition.

In some embodiments, a myeloid-specific immunomodulator (e.g., TGF-beta, losartan) can be used as modulating agent.

In some embodiments, the modulating agent can be an interferon (e.g., a Type I interferon).

In some embodiments, the modulating agent can be a BCL2 inhibitor.

In another aspect, embodiments disclosed herein provide a method of modulating a malignant signature comprising administering, to a population of cells comprising PDAC tumor cells, one or more agents capable of modulating the expression and/or activity of one or more biomarkers, expression programs, biologic programs, receptor-ligand interactions, cell state distribution, cell type distribution, or any combination thereof as in any of 1B-1D, 2A-2D, 3A-3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof. In some embodiments, the population of cells include malignant cells and/or non-malignant cells.

In certain example embodiments, the modulating agent induces and/or suppresses expression and/or activity of one or more biomarkers, expression programs, biologic programs, receptor-ligand interactions, cell state distribution, cell type distribution, or any combination thereof as in any of 1B-1D, 2A-2D, 3A-3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

### HDAC Inhibitor

In certain embodiments, the agent capable of modulating a signature as described herein is an HDAC inhibitor. Examples of HDAC inhibitors include hydroxamic acid derivatives, Short Chain Fatty Acids (SCFAs), cyclic tetrapeptides, benzamide derivatives, or electrophilic ketone derivatives, as defined herein. Specific non-limiting examples of HDAC inhibitors include: A) Hydroxamic acid derivatives selected from m-carboxycinnamic acid bishydroxamide (CBHA), Trichostatin A (TSA), Trichostatin C, Salicylhydroxamic Acid, Azelaic Bishydroxamic Acid (ABHA), Azelaic-1-Hydroxamate-9-Anilide (AAHA), 6-(3-Chlorophenylureido) carpoic Hydroxamic Acid (3Cl-UCHA), Oxamflatin, A-161906, Scriptaid, PXD-101, LAQ-824, CHAP, MW2796, and MW2996; B) Cyclic tetrapeptides selected from Trapoxin A, FR901228 (FK 228 or Depsipeptide), FR225497, Apicidin, CHAP, HC-Toxin, WF27082, and Chlamydocin; C) Short Chain Fatty Acids (SCFAs) selected from Sodium Butyrate, Isovalerate, Valerate, 4 Phenylbutyrate (4-PBA), Phenylbutyrate (PB), Propionate, Butyramide, Isobutyramide, Phenylacetate, 3-Bromopropionate, Tributyrin, Valproic Acid and Valproate; D) Benzamide Derivatives selected from C 1-994, MS-27-275 (MS-275) and a 3′-amino derivative of MS-27-275; E) Electrophilic Ketone Derivatives selected from a trifluoromethyl ketone and an α-keto amide such as an N-methyl-α-ketoamide; and F) Miscellaneous HDAC inhibitors including natural products, psammaplins and Depudecin.

Additional examples of HDAC inhibitors include vorinostat, romidepsin, chidamide, panobinostat, belinostat, mocetinostat, abexinostat, entinostat, resminostat, givinostat, quisinostat, CI-994, BML-210, M344, NVP-LAQ824, suberoylanilide hydroxamic acid (SAHA), MS-275, TSA, LAQ-824, trapoxin, depsipeptide, and tacedinaline.

Further examples of HDAC inhibitors include trichostatin A (TSA) ((R,2E,4E)-7-(4-(dimethylamino)phenyl)-N-hydroxy-4,6-dimethyl-7-oxohepta-2,4-dienamide); sulfonamides such as oxamflatin ((E)-N-hydroxy-5-(3-(phenylsulfonamido)phenyl)pent-2-en-4-ynamide). Other hydroxamic-acid-sulfonamide inhibitors of histone deacetylase are described in: Lavoie et al. (2001) Bioorg. Med. Chem. Lett.11:2847⁻⁵⁰; Bouchain et al. (2003) J. Med. Chem. 846:820-830; Bouchain et al. (2003) Curr. Med. Chem. 10:2359-2372; Marson et al. (2004) Bioorg. Med. Chem. Lett. 14:2477-2481; Finn et al. (2005) Helv. Chim. Acta 88:1630-1657; International Patent Publication Nos. WO 2002/030879, WO 2003/082288, WO 2005/0011661,; WO 2005/108367, WO 2006123121, WO 2006/017214, WO 2006/017215, and US Patent Publication No. 2005/0234033. Other structural classes of histone deacetylase inhibitors include short chain fatty acids, cyclic peptides, and benzamides. Acharya et al. (2005) Mol. Pharmacol. 68:917-932.

Other examples of HDAC inhibitors include those disclosed in, e.g., Dokmanovic et al. (2007) Mol. Cancer. Res. 5:981; U.S. Pat. Nos. 7,642,275; 7,683,185; 7,732,475; 7,737,184; 7,741,494; 7,772,245; 7,795,304; 7,799,825; 7,803,800; 7,842,727; 7,842,835; U.S. Patent Publication No. 2010/0317739; U.S. Patent Publication No. 2010/0311794; U.S. Patent Publication No. 2010/0310500; U.S. Patent Publication No. 2010/0292320; and U.S. Patent Publication No. 2010/0291003.

### CDK4/6 Inhibitor

In certain embodiments, the agent capable of modulating a signature as described herein is a cell cycle inhibitor (see e.g., Dickson and Schwartz, Development of cell-cycle inhibitors for cancer therapy, Curr Oncol. 2009 March; 16(2): 36-43). In one embodiment, the agent capable of modulating a signature as described herein is a CDK4/6 inhibitor, such as LEE011, palbociclib (PD-0332991), and Abemaciclib (LY2835219) (see, e.g., U.S. Pat. No. 9,259,399B2; International Patent Publication No. WO 2016/025650A1; US Patent Publication No. 2014/0031325; US Patent Publication No. 2014/0080838; US Patent Publication No. 2013/0303543; US Patent Publication No. 2007/0027147; US Patent Publication No. 2003/0229026; US Patent Publication No 2004/0048915; US Patent Publication No. 2004/0006074; and US Patent Publication No. 2007/0179118, each of which is incorporated herein by reference in its entirety). Currently there are three CDK4/6 inhibitors that are either approved or in late-stage development: palbociclib (PD-0332991; Pfizer), ribociclib (LEE011; Novartis), and abemaciclib (LY2835219; Lilly) (see e.g., Hamilton and Infante, Targeting CDK4/6 in patients with cancer, Cancer Treatment Reviews, Volume 45, April 2016, Pages 129-138).

### Checkpoint Inhibitors

Because immune checkpoint inhibitors target the interactions between different cells in the tumor, their impact depends on multicellular circuits between malignant and non-malignant cells (Tirosh et al., 2016a). In principle, resistance can stem from different compartment of the tumor's ecosystem, for example, the proportion of different cell types (e.g., T cells, macrophages, fibroblasts), the intrinsic state of each cell (e.g., memory or dysfunctional T cell), and the impact of one cell on the proportions and states of other cells in the tumor (e.g., malignant cells inducing T cell dysfunction by expressing PD-L1 or promoting T cell memory formation by presenting neoantigens). These different facets are inter-connected through the cellular ecosystem: intrinsic cellular states control the expression of secreted factors and cell surface receptors that in turn affect the presence and state of other cells, and vice versa. In particular, brisk tumor infiltration with T cell has been associated with patient survival and improved immunotherapy responses (Fridman et al., 2012), but the determinants that dictate if a tumor will have high (“hot”) or low (“cold”) levels of T cell infiltration are only partially understood. Among multiple factors, malignant cells may play an important role in determining this phenotype (Spranger et al., 2015). Resolving this relationship with bulk genomics approaches has been challenging; single-cell RNA-seq (scRNA-seq) of tumors (Li et al., 2017; Patel et al., 2014; Tirosh et al., 2016a, 2016b; Venteicher et al., 2017) has the potential to shed light on a wide range of immune evasion mechanisms and immune suppression programs. In certain embodiments, a treatment may include inhibitors of HDAC and/or CDK4/6 and a checkpoint agonist. Immune checkpoint agonists may activate checkpoint signaling, for example, by binding to the checkpoint protein. The agonists may include a ligand (e.g., PD-L1). PD-1 agonist antibodies that mimic PD-1 ligand (PD-L1) have been described (see, e.g., US Patent Publication No. 2017/0088618A1; International Patent Publication No. WO 2018/053405 A1). Such agonist antibodies against any receptor described herein are applicable to the present invention.

### Antibodies

The term “antibody” is used interchangeably with the term “immunoglobulin” herein, and includes intact antibodies, fragments of antibodies, e.g., Fab, F(ab′)₂ fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)₂, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, 1gM antibodies exist in pentameric form, and IgA antibodies exist in monomeric, dimeric or multimeric form.

The term “IgG subclass” refers to the four subclasses of immunoglobulin class IgG-1 IgG1, IgG2, IgG3, and IgG4 that have been identified in humans and higher mammals by the heavy chains of the immunoglobulins, V1 -γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by R pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245⁻⁵⁵), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g., LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins-harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, SnM or less, 1 nM or less, or in embodiments 500 μM or less, 100 μM or less, 50 μM or less or 25 μM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity, but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)₂, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having VH and CH₁ domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a VH domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (VH) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. For example, an antagonist antibody may bind an antigen or antigen receptor and inhibit the ability to suppress a response. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

The disclosure also encompasses nucleic acid molecules, in particular those that inhibitiHDAC and/or CDK4/6. Exemplary nucleic acid molecules include aptamers, siRNA, artificial microRNA, interfering RNA or RNAi, dsRNA, ribozymes, antisense oligonucleotides, and DNA expression cassettes encoding said nucleic acid molecules. Preferably, the nucleic acid molecule is an antisense oligonucleotide. Antisense oligonucleotides (ASO) generally inhibit their target by binding target mRNA and sterically blocking expression by obstructing the ribosome. ASOs can also inhibit their target by binding target mRNA thus forming a DNA-RNA hybrid that can be a substance for RNase H. Preferred ASOs include Locked Nucleic Acid (LNA), Peptide Nucleic Acid (PNA), and morpholinos Preferably, the nucleic acid molecule is an RNAi molecule, i.e., RNA interference molecule. Preferred RNAi molecules include siRNA, shRNA, and artificial miRNA. The design and production of siRNA molecules is well known to one of skill in the art (e.g., Hajeri P B, Singh S K. Drug Discov Today. 2009 14(17-18):851-8). The nucleic acid molecule inhibitors may be chemically synthesized and provided directly to cells of interest. The nucleic acid compound may be provided to a cell as part of a gene delivery vehicle. Such a vehicle is preferably a liposome or a viral gene delivery vehicle.

### Genetic Modifying Agents

In certain embodiments, the one or more modulating agents may be a genetic modifying agent. In certain embodiments, the one or more modulating agents may be a genetic modifying agent. The genetic modifying agent may comprise a CRISPR system, a zinc finger nuclease system, a TALEN, a meganuclease or RNAi system. In some embodiments, a polynucleotide of the present invention described elsewhere herein can be modified using a genetic modifying agent (e.g., one or more genes as in any of 1B-1D, 2A-2D, 3A-3C, 3E, 5, 4B-4D, 5A-5C, 6A-6B, 7, 10, 11, 12, 16B-16E, Tables 2.1-2.6, 3, 4, and any combination thereof, one or more genes as in any of FIGS. 1B-1D, 2A-2D, 3A-3B, 3E, 5A-5C, 6A-6B, 7, 9C-9D, 14, 15A-15D, 16B, and Tables 2.1-2.6, 3 or 5; one or more genes as in any of FIGS. 1B-1D, 2A-2D, 4A-4F, 6A-6B, 9A-9B, 12, Table 7, or any combination thereof, and/or one or more genes as in any of FIGS. 1B-1D, 2A-2D, 3A-3C, 4A-4C, 5A-5C, 7, 9A-9D, 10, 11, 12, 14, 15A-15D, Table 2.1-2.6, and any combination thereof.

### CRISPR-Cas Modification

In some embodiments, a polynucleotide of the present invention described elsewhere herein can be modified using a CRISPR-Cas and/or Cas-based system.

In general, a CRISPR-Cas or CRISPR system as used in herein and in documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

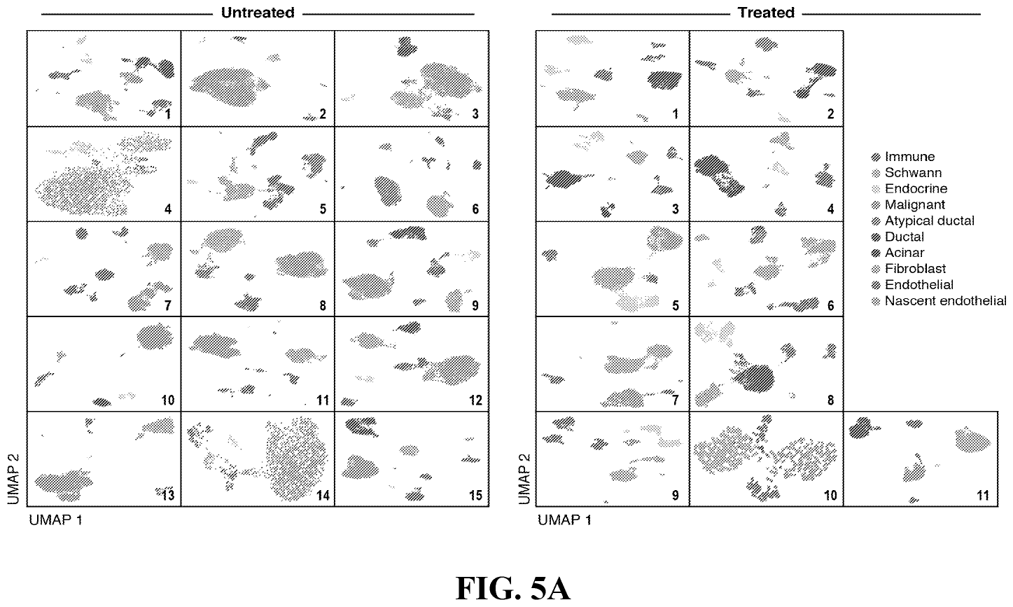

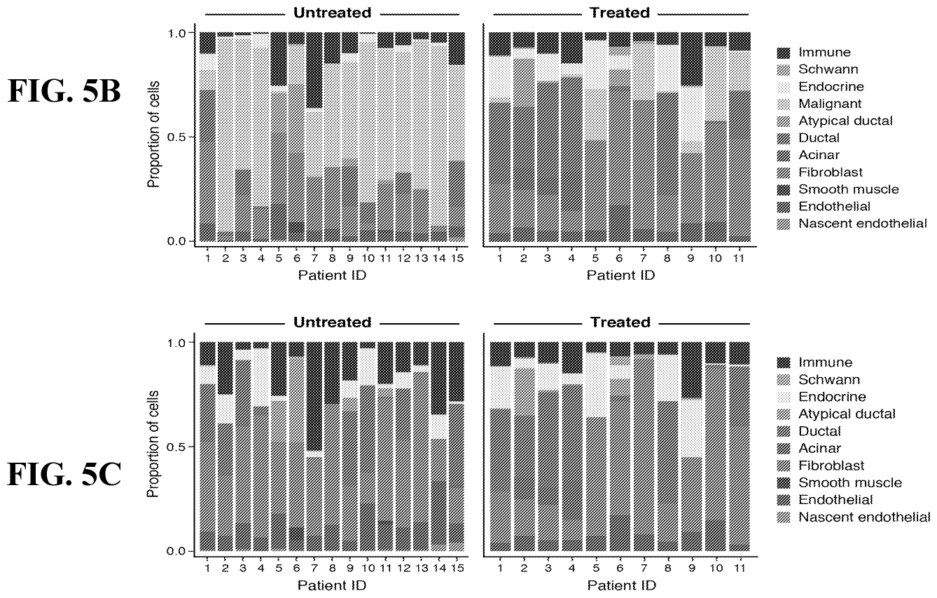

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically comprise a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374:20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374:20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), Cas12d (CasY), Cas12e (CasX), and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 September 12; 154(6):1380-1389), Cas12 (Liu et al. Nature Communications, 8, 2095 (2017), and Cas13 (International Patent Publication Nos. WO 2019/005884 and WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018.Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

In certain example embodiments, the base editing system may be an RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505⁻⁵ (2019), which is incorporated herein by reference.

### Prime Editors

In some embodiments, a polynucleotide of the present invention described elsewhere herein can be modified using a prime editing system. See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion and combinations thereof. Generally, a prime editing system, as exemplified by PE1, PE2, and PE3 (Id.), can include a reverse transcriptase fused or otherwise coupled or associated with an RNA-programmable nickase and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g., is a Cas9 nickase). In some embodiments, the Cas polypeptide is fused to the reverse transcriptase. In some embodiments, the Cas polypeptide is linked to the reverse transcriptase.

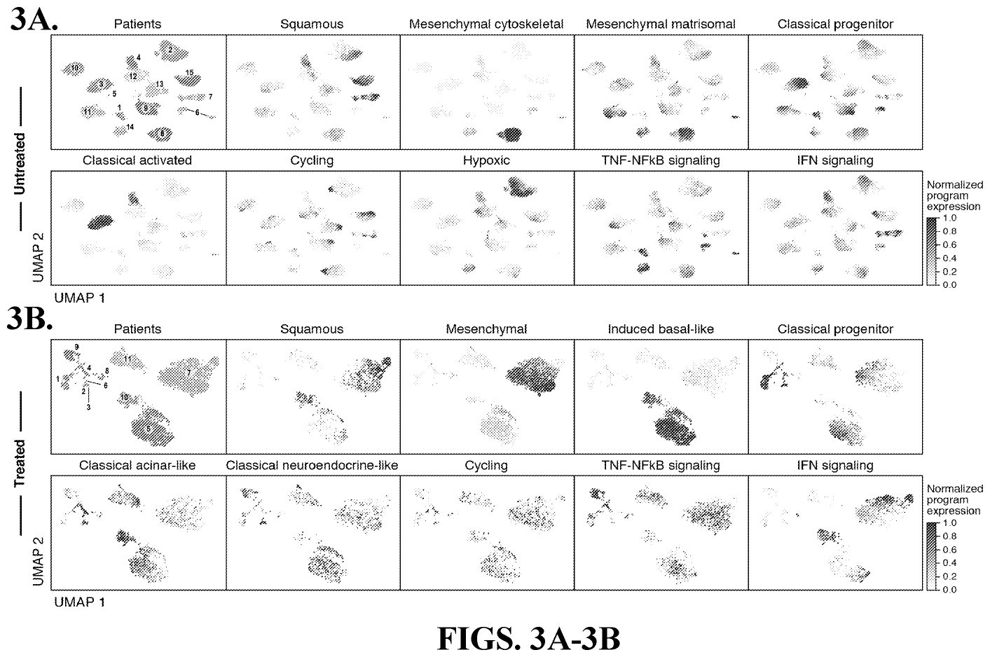

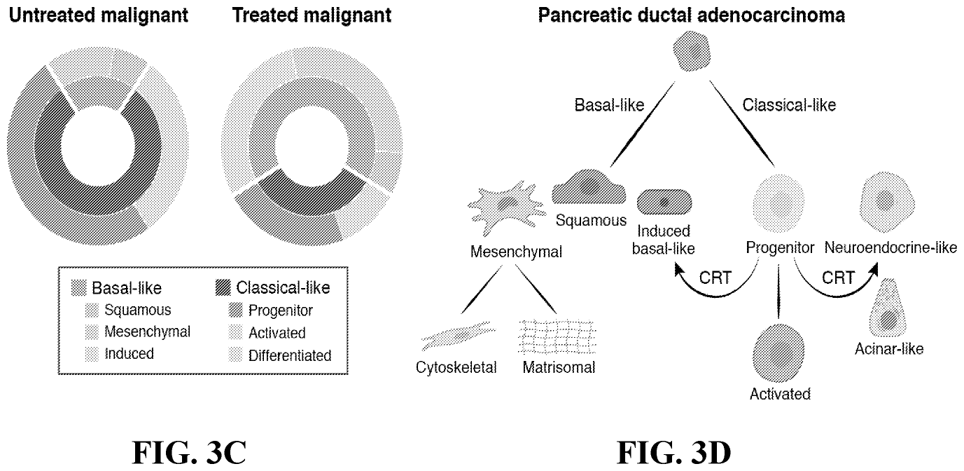

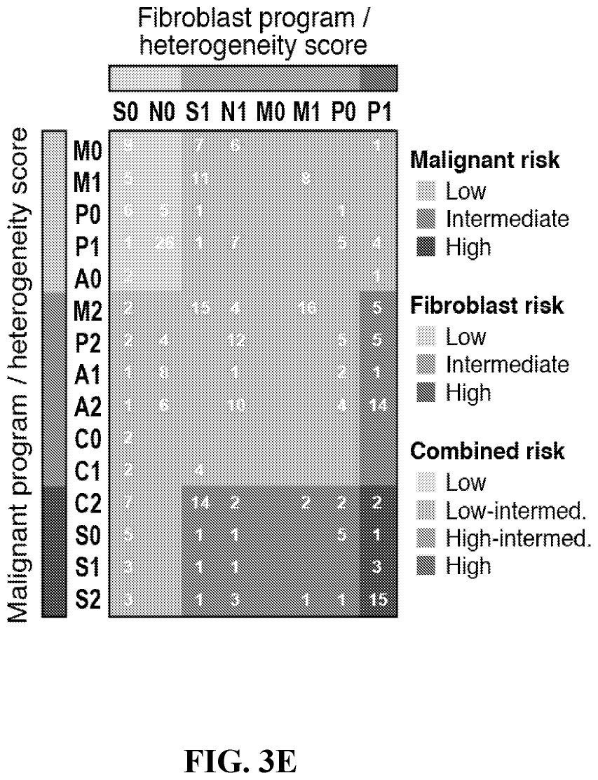

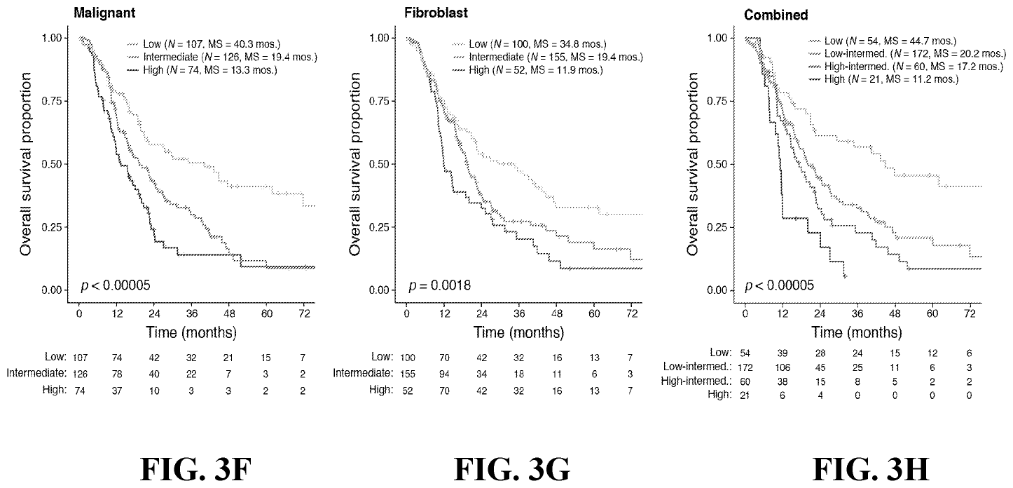

In some embodiments, the prime editing system can be a PE1 system or variant thereof, a PE2 system or variant thereof, or a PE3 (e.g., PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9(1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568⁻⁵⁷¹). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2⁻⁵ base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504⁻⁵¹⁷. Table 8 (from Gleditzsch et al. 2019) below shows several Cas polypeptides and the PAM sequence they recognize.

TABLE 8
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpfl and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481⁻⁵. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52⁻⁵⁷. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016.Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:1) or PKKKRKVEAS (SEQ ID NO:2); the NLS from nucleoplasmin (e.g., the nucleoplasmin bipartite NLS with the sequence KRPAATKKAGQAKKKK (SEQ ID NO:3)); the c-myc NLS having the amino acid sequence PAAKRVKLD (SEQ ID NO:4) or RQRRNELKRSP (SEQ ID NO:5); the hRNPA1 M9 NLS having the sequence NQSSNFGPMKGGNFGGRSSGPYGGGGQYFAKPRNQGGY (SEQ ID NO:6); the sequence RMRIZFKNKGKDTAELRRRRVEVSVELRKAKKDEQILKRRNV (SEQ ID NO:7) of the IBB domain from importin-alpha; the sequences VSRKRPRP (SEQ ID NO:8) and PPKKARED (SEQ ID NO:9) of the myoma T protein; the sequence PQPKKKPL (SEQ ID NO:10) of human p53; the sequence SALIKKKKKMAP (SEQ ID NO:11) of mouse c-abl IV; the sequences DRLRR (SEQ ID NO:12) and PKQKKRK (SEQ ID NO:13) of the influenza virus NS1; the sequence RKLKKKIKKL (SEQ ID NO:14) of the Hepatitis virus delta antigen; the sequence REKKKFLKRR (SEQ ID NO:15) of the mouse Mx1 protein; the sequence KRKGDEVDGVDEVAKKKSKK (SEQ ID NO:16) of the human poly(ADP-ribose) polymerase; and the sequence RKCLQAGMNLEARKTKK (SEQ ID NO:17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to a nucleotide deaminase or catalytic domain thereof. When such a guide forms a CRISPR complex (e.g., CRISPR-Cas protein binding to guide and target), the adapter proteins bind and the nucleotide deaminase or catalytic domain thereof associated with the adapter protein is positioned in a spatial orientation which is advantageous for the attributed function to be effective.

The skilled person will understand that modifications to the guide which allow for binding of the adapter+ nucleotide deaminase, but not proper positioning of the adapter+nucleotide deaminase (e.g., due to steric hindrance within the three-dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, a composition for engineering cells comprises a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include a sequence that corresponds to both, a first site on the target sequence that is cleaved in a first Cas protein mediated event, and a second site on the target sequence that is cleaved in a second Cas protein mediated event.

In certain embodiments, the template nucleic acid can include a sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include a sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include a sequence which, when integrated, results in decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include a sequence which results in a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)z, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 18)
M D P I R S R T P S P A R E L L S G P Q P D G V Q P T A D R G V S P P A G
G P L D G L P A R R T M S R T R L P S P P A P S P A F S A D S F S D L L R Q F D P S L
F N T S L F D S L P P F G A H H T E A A T G E W D E V Q S G L R A A D A P P P T M R
V A V T A A R P P R A K P A P R R R A A Q P S D A S P A A Q V D L R T L G Y S Q Q
Q Q E K I K P K V R S T V A Q H H E A L V G H G F T H A H I V A L S Q H P A A L G
T V A V K Y Q D M I A A L P E A T H E A I V G V G K Q W S G A R A L E A L L T V A
G E L R G P P L Q L D T G Q L L K I A K R G G V T A V E A V H A W R N A L T G A P
L N

An exemplary amino acid sequence of a C-terminal capping region is:

(SEQ ID NO: 19)
R P A L E S I V A Q L S R P D P A L A A L T N D H L V A L A C L G G R P A
L D A V K K G L P H A P A L I K R T N R R I P E R T S H R V A D H A Q V V R V L G F
F Q C H S H P A Q A F D D A M T Q F G M S R H G L L Q L F R R V G V T E L E A R S
G T L P P A S Q R W D R I L Q A S G M K R A K P S P T S T Q T P D Q A S L H A F A D
S L E R D L D A P S P M H E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments, the effector domain is an enhancer of transcription (i.e., an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15⁻⁵⁰ nucleotides in length (e.g., each complementary sequence of the double stranded siRNA is about 15⁻⁵⁰ nucleotides in length, and the double stranded siRNA is about 15⁻⁵⁰ base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g., about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated herein by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Combination Therapies

Described herein are combination therapies that can be used in a subject in need thereof having PDAC. in some embodiments, the combination therapy can include detection and and/or monitoring a PDAC tumor signature described elsewhere herein. In some embodiments, the combination therapy can include neoadjuvant treatment, PDAC tumor resection, administration of a PDAC signature modulating agent, a post neoadjuvant therapy, or a combination thereof.

### Phased Combination Therapy

In certain embodiments, a subject in need thereof is treated with a combination therapy, which may be a phased combination therapy. Phased combination therapies are combination therapies are those that contain various treatment phases where each phase can incorporate a different therapy approach. In some embodiments, the initiation of each phase can be dictated by achieving a particular milestone, such as a specific signature, subject response, time, number of doses, or other predetermined standard.

In some embodiments, the phased combination therapy can include administration of one or more PDAC modulators as described elsewhere herein, PDAC tumor resection, neoadjuvant administration, or a combination thereof. In some embodiments, the phased combination therapy can include detecting and/or monitoring a PDAC signature described in greater detail elsewhere herein.

In some embodiments, phased combination therapy may be a treatment regimen comprising checkpoint inhibition followed by a CDK4/6 inhibitor, an HDAC inhibitor, an/or checkpoint inhibitor combination. Checkpoint inhibitors may be administered at regular intervals, for example, daily, weekly, every two weeks, every month. The combination therapy may be administered when a signature disclosed herein is detected. This may be after two weeks to six months after the initial checkpoint inhibition. The immunotherapy may be adoptive cell transfer therapy, as described herein or may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Specific check point inhibitors include, but are not limited to anti-CTLA4 antibodies (e.g., Ipilimumab), anti-PD-1 antibodies (e.g., Nivolumab, Pembrolizumab), and anti-PD-L1 antibodies (e.g., Atezolizumab). Dosages for the immunotherapy and/or CDK4/6 inhibitors may be determined according to the standard of care for each therapy and may be incorporated into the standard of care (see, e.g., Rivalland et al., Standard of care in immunotherapy trials: Challenges and considerations, Hum Vaccin Immunother. 2017 July; 13(9): 2164-2178; and Pernas et al., CDK4/6 inhibition in breast cancer: current practice and future directions, Ther Adv Med Oncol. 2018). The standard of care is the current treatment that is accepted by medical experts as a proper treatment for a certain type of disease and that is widely used by healthcare professionals. Standard or care is also called best practice, standard medical care, and standard therapy.

### Pharmaceutical Formulations and Administration

### Administration

It will be appreciated that administration of therapeutic entities in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences (15th ed, Mack Publishing Company, Easton, PA (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Baldrick P. “Pharmaceutical excipient development: the need for preclinical guidance.” Regul. Toxicol Pharmacol. 32(2):210-8 (2000), Wang W. “Lyophilization and development of solid protein pharmaceuticals.” Int. J. Pharm. 203(1-2):1-60 (2000), Charman W N “Lipids, lipophilic drugs, and oral drug delivery-some emerging concepts.” J Pharm Sci. 89(8):967-78 (2000), Powell et al. “Compendium of excipients for parenteral formulations” PDA J Pharm Sci Technol. 52:238-311 (1998) and the citations therein for additional information related to formulations, excipients and carriers well known to pharmaceutical chemists.

The medicaments of the invention are prepared in a manner known to those skilled in the art, for example, by means of conventional dissolving, lyophilizing, mixing, granulating or confectioning processes. Methods well known in the art for making formulations are found, for example, in Remington: The Science and Practice of Pharmacy, 20th ed., ed. A. R. Gennaro, 2000, Lippincott Williams & Wilkins, Philadelphia, and Encyclopedia of Pharmaceutical Technology, eds. J. Swarbrick and J. C. Boylan, 1988-1999, Marcel Dekker, New York.

Administration of medicaments of the invention may be by any suitable means that results in a compound concentration that is effective for treating or inhibiting (e.g., by delaying) the development of a disease. The compound is admixed with a suitable carrier substance, e.g., a pharmaceutically acceptable excipient that preserves the therapeutic properties of the compound with which it is administered. One exemplary pharmaceutically acceptable excipient is physiological saline. The suitable carrier substance is generally present in an amount of 1-95% by weight of the total weight of the medicament. The medicament may be provided in a dosage form that is suitable for administration. Thus, the medicament may be in form of, e.g., tablets, capsules, pills, powders, granulates, suspensions, emulsions, solutions, gels including hydrogels, pastes, ointments, creams, plasters, drenches, delivery devices, injectables, implants, sprays, or aerosols.

The agents disclosed herein may be used in a pharmaceutical composition when combined with a pharmaceutically acceptable carrier. Such compositions comprise a therapeutically-effective amount of the agent and a pharmaceutically acceptable carrier. Such a composition may also further comprise (in addition to an agent and a carrier) diluents, fillers, salts, buffers, stabilizers, solubilizers, and other materials well known in the art. Compositions comprising the agent can be administered in the form of salts provided the salts are pharmaceutically acceptable. Salts may be prepared using standard procedures known to those skilled in the art of synthetic organic chemistry.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Various delivery systems are known and can be used to administer the pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321:574 (1989) and a semi-permeable polymeric material (See, for example, Howard, et al., J. Neurosurg. 71:105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

The amount of the agents which will be effective in the treatment of a particular disorder or condition will depend on the nature of the disorder or condition, and may be determined by standard clinical techniques by those of skill within the art. In addition, in vitro assays may optionally be employed to help identify optimal dosage ranges. The precise dose to be employed in the formulation will also depend on the route of administration, and the overall seriousness of the disease or disorder, and should be decided according to the judgment of the practitioner and each patient's circumstances. Ultimately, the attending physician will decide the amount of the agent with which to treat each individual patient. In certain embodiments, the attending physician will administer low doses of the agent and observe the patient's response. Larger doses of the agent may be administered until the optimal therapeutic effect is obtained for the patient, and at that point the dosage is not increased further. In general, the daily dose range lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases. In certain embodiments, suitable dosage ranges for intravenous administration of the agent are generally about 5⁻⁵⁰⁰ micrograms (g) of active compound per kilogram (Kg) body weight. Suitable dosage ranges for intranasal administration are generally about 0.01 pg/kg body weight to 1 mg/kg body weight. In certain embodiments, a composition containing an agent of the present invention is subcutaneously injected in adult patients with dose ranges of approximately 5 to 5000 μg/human and preferably approximately 5 to 500 μg/human as a single dose. It is desirable to administer this dosage 1 to 3 times daily. Effective doses may be extrapolated from dose-response curves derived from in vitro or animal model test systems. Suppositories generally contain active ingredient in the range of 0.5% to 10% by weight; oral formulations preferably contain 10% to 95% active ingredient. Ultimately the attending physician will decide on the appropriate duration of therapy using compositions of the present invention. Dosage will also vary according to the age, weight and response of the individual patient.

Methods for administering antibodies for therapeutic use is well known to one skilled in the art. In certain embodiments, small particle aerosols of antibodies or fragments thereof may be administered (see e.g., Piazza et al., J. Infect. Dis., Vol. 166, pp. 1422-1424, 1992; and Brown, Aerosol Science and Technology, Vol. 24, pp. 45-56, 1996). In certain embodiments, antibodies are administered in metered-dose propellant driven aerosols. In preferred embodiments, antibodies are used as agonists to depress inflammatory diseases or allergen-induced asthmatic responses. In certain embodiments, antibodies may be administered in liposomes, i.e., immunoliposomes (see, e.g., Maruyama et al., Biochim. Biophys. Acta, Vol. 1234, pp. 74-80, 1995). In certain embodiments, immunoconjugates, immunoliposomes or immunomicrospheres containing an agent of the present invention is administered by inhalation.

In certain embodiments, antibodies may be topically administered to mucosa, such as the oropharynx, nasal cavity, respiratory tract, gastrointestinal tract, eye such as the conjunctival mucosa, vagina, urogenital mucosa, or for dermal application. In certain embodiments, antibodies are administered to the nasal, bronchial or pulmonary mucosa. In order to obtain optimal delivery of the antibodies to the pulmonary cavity in particular, it may be advantageous to add a surfactant such as a phosphoglyceride, e.g., phosphatidylcholine, and/or a hydrophilic or hydrophobic complex of a positively or negatively charged excipient and a charged antibody of the opposite charge.

Other excipients suitable for pharmaceutical compositions intended for delivery of antibodies to the respiratory tract mucosa may be a) carbohydrates, e.g., monosaccharides such as fructose, galactose, glucose. D-mannose, sorbiose, and the like; disaccharides, such as lactose, trehalose, cellobiose, and the like; cyclodextrins, such as 2-hydroxypropyl-β-cyclodextrin; and polysaccharides, such as raffinose, maltodextrins, dextrans, and the like; b) amino acids, such as glycine, arginine, aspartic acid, glutamic acid, cysteine, lysine and the like; c) organic salts prepared from organic acids and bases, such as sodium citrate, sodium ascorbate, magnesium gluconate, sodium gluconate, tromethamine hydrochloride, and the like: d) peptides and proteins, such as aspartame, human serum albumin, gelatin, and the like; e) alditols, such mannitol, xylitol, and the like, and f) polycationic polymers, such as chitosan or a chitosan salt or derivative.

For dermal application, the antibodies of the present invention may suitably be formulated with one or more of the following excipients: solvents, buffering agents, preservatives, humectants, chelating agents, antioxidants, stabilizers, emulsifying agents, suspending agents, gel-forming agents, ointment bases, penetration enhancers, and skin protective agents.

Examples of solvents are e.g. water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rapeseed oil, sesame oil, soybean oil, sunflower oil, and tea seed oil), mineral oils, fatty oils, liquid paraffin, polyethylene glycols, propylene glycols, glycerol, liquid polyalkylsiloxanes, and mixtures thereof.

Examples of buffering agents are e.g. citric acid, acetic acid, tartaric acid, lactic acid, hydrogenphosphoric acid, diethyl amine etc. Suitable examples of preservatives for use in compositions are parabenes, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g., soybean lecithin, sorbitan monooleate derivatives: wool fats; wool alcohols; sorbitan esters; monoglycerides; fatty alcohols; fatty acid esters (e.g. triglycerides of fatty acids); and mixtures thereof.

Examples of suspending agents are e.g., celluloses and cellulose derivatives such as, e.g., carboxymethyl cellulose, hydroxyethylcellulose, hydroxypropylcellulose, hydroxypropylmethylcellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminum, zinc soaps, glycerol, propylene glycol, tragacanth, carboxyvinyl polymers, magnesium-aluminum silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), and alginates including propylene glycol alginate.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes. Examples of hydrophilic ointment bases are solid macrogols (polyethylene glycols). Other examples of ointment bases are triethanolamine soaps, sulphated fatty alcohol and polysorbates.

Examples of other excipients are polymers such as carmelose, sodium carmelose, hydroxypropylmethylcellulose, hydroxyethylcellulose, hydroxypropylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and chitosans.

The dose of antibody required in humans to be effective in the treatment or prevention of allergic inflammation differs with the type and severity of the allergic condition to be treated, the type of allergen, the age and condition of the patient, etc. Typical doses of antibody to be administered are in the range of 1 μg to 1 g, preferably 1-1000 μg, more preferably 2⁻⁵⁰⁰, even more preferably 5⁻⁵⁰, most preferably 10-20 μg per unit dosage form. In certain embodiments, infusion of antibodies of the present invention may range from 10⁻⁵⁰⁰ mg/m2.

There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. The currently preferred in vivo gene transfer techniques include transfection with viral (typically retroviral) vectors and viral coat protein-liposome mediated transfection.

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes, hours, days, months, years or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a PDAC signature modulating agent or other PDAC treatment or agent described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a PDAC, neoadjuvant resistant malignant PDAC cells, and/or a symptom thereof. As used herein, “agent” generally refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are to treat PDAC or symptom thereof, to modulate or maintain a PDAC tumor signature, or a combination thereof.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140,150, 160,170,180, 190,200,210,220,230,240,250,260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, M, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1X101/mL, 1X1020/mL or more, such as about 1X101/mL, 1X102/mL, 1X103/mL, 1X104/mL, 1X105/mL, 1X106/mL, 1X107/mL, 1X108/mL, 1X109/mL, 1X1010/mL, 1X1011/mL, 1X1012/mL, 1X1013/mL, 1X1014/mL, 1X1015/mL, 1X1016/mL, 1X1017/mL, 1X1018/mL, 1X1019/mL, to/or about 1X1020/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1X101 particles per pL, nL, L, mL, or L to 1X1020/particles per pL, nL, L, mL, or L or more, such as about 1X101, 1X102, 1X103, 1X104, 1X105, 1X106, 1X107, 1X108, 1X109, 1X1010, 1X1011, 1X1012, 1X1013, 1X1014, 1X1015, 1X1016, 1X1017, 1X1018, 1X1019, to/or about 1X1020 particles per pL, nL, L, mL, or L. In some embodiments, the effective titer can be about 1X101 transforming units per pL, nL, L, mL, or L to 1X1020/transforming units per pL, nL, L, mL, or L or more, such as about 1X101, 1X102, 1X103, 1X104, 1X105, 1X106, 1X107, 1X108, 1X109, 1X1010, 1X1011, 1X1012, 1X1013, 1X1014, 1X1015, 1X1016, 1X1017, 1X1018, 1X1019, to/or about 1X1020 transforming units per pL, nL, L, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, M D, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Methods of Screening for PDAC Signature Modulating Agents

Described in several embodiments herein of screening for agents capable of modulating a PDAC signature as described in greater detail elsewhere herein. Described in certain example embodiments herein are methods of screening for one or more agents capable of modulating a PDAC malignant cell state comprising:
contacting a cell population comprising PDAC malignant cells having an initial cell state with a test modulating agent or library of modulating agents;
determining a fraction of malignant cells having a desired cell state and an undesired cell state;
selecting modulating agents that shift the initial PDAC malignant cell state to a desired cell state or prevent the initial PDAC malignant cell state to shift from a desired initial state, such that the fraction of PDAC malignant cells in the cell population having a desired cell state is above a set cutoff limit.

In certain example embodiments, the desired PDAC malignant cell state is a classic progenitor cell state or a mesenchymal matrisomal cell state.

In certain example embodiments, the cell population is obtained from a subject to be treated.

The methods of nucleic acid analysis described in greater detail elsewhere herein (see e.g., section on methods of diagnosing, prognosing and/or treating PDAC) can be utilized for evaluating environmental stress and/or state, for screening of chemical and/or biologic libraries, and to screen or identify structural, syntenic, genomic, and/or organism and species variations. Aspects of the present disclosure relate to the correlation of an environmental stress or state with the spatial proximity and/or epigenetic profile of the nucleic acids in a sample of cells, for example a culture of cells, can be exposed to an environmental stress, such as but not limited to heat shock, osmolarity, hypoxia, cold, oxidative stress, radiation, starvation, a chemical or biologic (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value.

In some embodiments, the disclosed methods can be used to screen chemical and/or biologic libraries for agents that modulate chromatin architecture epigenetic profiles, and/or relationships thereof. By exposing cells, or fractions thereof, tissues, or even whole animals, to different members of the chemical libraries, and performing the methods described herein, different members of a chemical library can be screened for their effect on architecture epigenetic profiles, and/or relationships thereof simultaneously in a relatively short amount of time, for example using a high throughput method.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more phenotypic aspects of the cell or cell population by the candidate agent, thereby identifying the agent. The phenotypic aspects of the cell or cell population that is modulated may be a gene signature or biological program specific to a cell type or cell phenotype or phenotype specific to a population of cells (e.g., an inflammatory phenotype or suppressive immune phenotype). In certain embodiments, steps can include administering candidate modulating agents to cells, detecting identified cell (sub)populations for changes in signatures, or identifying relative changes in cell (sub) populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%,²⁰⁰%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on immune phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells. In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator compounds. A combinatorial chemical library may be a collection of diverse chemical compounds generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given compound length (for example the number of amino acids in a polypeptide compound). Millions of chemical compounds can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or biological programs of the present invention may be used to screen for drugs that reduce the signature or biological program in cells as described herein. The signature or biological program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or biological program of the present invention in silico.

### Kits

Any of the compounds, compositions, formulations, particles, cells, devices, or any combination thereof described herein, or a combination thereof can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agents) contained in the kit are administered simultaneously, the combination kit can contain the active agents in a single formulation, such as a pharmaceutical formulation, (e.g., a tablet) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds, compositions, formulations, particles, cells, described herein or a combination thereof contained therein, safety information regarding the content of the compounds, compositions, formulations (e.g., pharmaceutical formulations), particles, and cells described herein or a combination thereof contained therein, information regarding the dosages, indications for use, and/or recommended treatment regimen(s) for the compound(s) and/or pharmaceutical formulations contained therein. In some embodiments, the instructions can provide directions for administering the compounds, compositions, formulations, particles, and cells described herein or a combination thereof to a subject in need thereof.

In some embodiments, the subject in need thereof is in need of a treatment or prevention for a pancreatic disease or a symptom thereof. In some embodiments, the pancreatic disease can be a pancreatic cancer. In some embodiments, the pancreatic disease is PDAC. In some embodiments, the instructions provide that the subject in need thereof or a tissue and/or cell(s) from said subject, to which the compounds, compositions, formulations, particles, cells, described herein or a combination thereof can be administered, has one or more PDAC signatures described herein. In some embodiments, the instructions and/or a label includes diagnostic, prognostic and/or PDAC treatment guidance based on one or more detected PDAC signatures described herein.

Further embodiments are illustrated in the following Examples which are given for illustrative purposes only and are not intended to limit the scope of the invention.

### EXAMPLES

### Example 1

### Single-Nucleus RNA-Seq Accurately Represents the Malignant and Non-Malignant Compartments of Human PDAC Tumors

snRNA-seq was performed on flash frozen, histologically-confirmed, primary PDAC specimens from patients (n=26) with respectable or borderline-respectable disease, who underwent surgical resection with (n=11) or without (n=15) neoadjuvant CRT (FIG. 1A; Table 1) and analyzed 138,547 high quality single nucleus profiles (Methods). Single nucleus profiles were separately grouped by treatment status from all patients by unsupervised clustering and annotated cell subsets using known cell type-specific gene signatures (FIG. 1B; FIGS. 5A-5C; Methods). The identity of malignant cells was confirmed by inferred Copy Number Aberrations (CNAs) (FIG. 6A)²⁹. The frequencies of inferred chromosome arm-level somatic CNAs in the malignant cells of these patient samples were comparable to those in The Cancer Genome Atlas (TCGA) pancreatic adenocarcinoma cohort as assessed by single nucleotide polymorphisms and whole-exome sequencing (FIG. 6B)¹¹. Among non-malignant cells, all major cell types known to compose exocrine pancreatic tumors were identified (FIGS. 1B-1C). The presence of a subset of atypical ductal-like cells (CFTR^{high};KRT19^{high};CNA^{low}) was noted (FIG. 1B) that are unlikely to be doublets based on their typical number of unique molecular identifiers and could be further explored in future studies as possible precursors to invasive cancer cells.

TABLE 1

| | | | | | | | | | | | | |
|---|---|---|---|---|---|---|---|---|---|---|---|---|
| | Age | | | | | | | Status | | | | 10× |
| | (de- | | Stage/ | | His- | | | Last | PFS | OS | Storage | Chem- |
| ID | cade) | Sex | Grade | Margin | tology | Adjuvant | Neoadjuvant | FUP | (d) | (d) | time (d) | istry |

Untreated (U)

| | | | | | | | | | | | | |
|---|---|---|---|---|---|---|---|---|---|---|---|---|
| PDAC_U_1 | 20s | F | T3N1M0/ | R1 | | FOLFIRINOX/ | None | MET | 70 | 892 | 638 | v3 |
| | | | g2-3 | | | Gem/RT | | | | | | |
| PDAC_U_2 | 60s | F | T3N2M0/ | R1 | AS | FOLFIRINOX/ | None | LR/DWD | 78 | 297 | 17 | v3 |
| | | | g2 | | | RT | | | | | | |
| PDAC_U_3 | 60s | F | T3N0M0/ | R0 | | Gem/cape/RT | None | MET | 578 | 649 | 341 | v3 |
| | | | gX | | | | | | | | | |
| PDAC_U_4 | 60s | M | T3N1M0/ | R0 | | Gem/cape/RT | None | LR/DWD | 267 | 538 | 634 | v2 |
| | | | g3 | | | | | | | | | |
| PDAC_U_5 | 60s | M | T3N1M0/ | R1 | | Gem/cape/RT | None | NED | 282 | 282 | 661 | v3 |
| | | | g3 | | | | | | | | | |
| PDAC_U_6 | 60s | M | T2N2M0/ | R0 | | Gem/cape | None | NED | 151 | 151 | 220 | v3 |
| | | | g2 | | | | | | | | | |
| PDAC_U_7 | 70s | M | T3N2M0/ | R0 | AS | None | None | MET/ | 208 | 563 | 384 | v3 |
| | | | g2 | | | | | DWD | | | | |
| PDAC_U_8 | 70s | M | T2N1M0/ | R0 | | Gem/ | None | MET | 35 | 186 | 62 | v3 |
| | | | g2 | | | abraxane | | | | | | |
| PDAC_U_9 | 70s | M | T3N0M0/ | R1 | | None | None | DWOD | 494 | 494 | 472 | v3 |
| | | | g2-3 | | | | | | | | | |
| PDAC_U_10 | 70s | F | T2N1M0/ | R1 | | Gem/cape/RT | None | NED | 644 | 644 | 393 | v3 |
| | | | g2 | | | | | | | | | |
| PDAC_U_11 | 70s | F | T2N1M0/ | R1 | | FOLFIRINOX/ | None | DWOD | 172 | 172 | 64 | v3 |
| | | | g2 | | | RT | | | | | | |
| PDAC_U_12 | 70s | M | T2N0M0/ | R1 | | FOLFIRINOX | None | LR | 329 | 555 | 135 | v2 |
| | | | g2-3 | | | | | | | | | |
| PDAC_U_13 | 70s | M | T3N0M0/ | R1 | | Gem/ | None | NED | 224 | 224 | 106 | v3 |
| | | | g2 | | | abraxane | | | | | | |
| PDAC_U_14 | 80s | F | T2N0M0/ | R0 | | Gem | None | NED | 225 | 225 | 38 | v2 |
| | | | g2 | | | | | | | | | |
| PDAC_U_15 | 80s | M | T3N2M0/ | R1 | AS | None | None | NED | 35 | 35 | 148 | v3 |
| | | | g3 | | | | | | | | | |

Treated (T)

| | | | | | | | | | | | | |
|---|---|---|---|---|---|---|---|---|---|---|---|---|
| PDAC_T_1 | 30s | F | ypT2N0M0/ | R0 | BRCA2 | FOLFIRINOX/ | Olaparib | NED | 301 | 301 | 69 | v3 |
| | | | g2 | | germ | RT | | | | | | |
| PDAC_T_2 | 50s | M | ypT1cN0M0/ | R1 | BRCA2 | FOLFIRINOX/ | FOLFIRINOX | MET | 285 | 285 | 170 | v3 |
| | | | g3 | | germ | RT | | | | | | |
| PDAC_T_3 | 50s | F | ypT3N0M0/ | R0 | | FOLFIRINOX/ | | NED | 1255 | 1255 | 1121 | v3 |
| | | | g2 | | | RT | | | | | | |
| PDAC_T_4 | 60s | F | ypT3N0M0/ | R0 | | FOLFIRINOX/ | | NED | 1342 | 1342 | 1142 | v3 |
| | | | g2 | | | RT | | | | | | |
| PDAC_T_5 | 60s | F | ypT2N0M0/ | R1 | | FOLFIRINOX/ | Gem/ | LR | 187 | 433 | 104 | v2 |
| | | | gX | | | RT | abraxane | | | | | |
| PDAC_T_6 | 60s | F | ypT1aN1M0/ | R0 | | FOLFIRINOX/ | Nivolumab | NED | 264 | 264 | 58 | v3 |
| | | | gX | | | RT/nivo | | | | | | |
| PDAC_T_7 | 60s | M | ypT2N2M0/ | R0 | AS | FOLFIRINOX/ | | MET | 34 | 34 | 112 | v2 |
| | | | g3 | | | RT | | | | | | |
| PDAC_T_8 | 60s | M | ypT1cN0M0/ | R0 | | FOLFIRINOX/ | Gem/ | MET | 117 | 345 | 56 | v2 |
| | | | g2 | | | RT | abraxane | | | | | |
| PDAC_T_9 | 70s | M | ypT3N0M0/ | R0 | | FOLFIRINOX/ | | LR/DWD | 258 | 362 | 905 | v3 |
| | | | g2 | | | RT/losartan | | | | | | |
| PDAC_T_10 | 70s | M | ypT3N0M0/ | R0 | | RT/cape | Gemcitabine | Unknown | | | 2389 | v2 |
| | | | g2 | | | | | | | | | |
| PDAC_T_11 | 70s | F | ypT2N1M0/ | R0 | | RT | Gemcitabine | MET | 110 | 185 | 365 | v3 |
| | | | g3 | | | | | | | | | |

Abbreviations: AS, adenosquamous; MET, distant metastases; LR, local recurrence; DWD, dead with disease; DWOD, dead without evidence of disease; NED, no evidence of disease; Gem, gemcitabine; Cape, capecitabine; RT, radiotherapy

Examining treatment-naive and neoadjuvant-treated specimens separately (FIG. 1C)⁴⁴, non-malignant cell subsets primarily partitioned by cell type with substantial inter-patient mixing, whereas malignant cells partitioned by patient, as previously reported for other tumor types^{29,31,32,45,46}. Among non-malignant cells, annotated diverse immune, endocrine, and acinar cells, and their cell subsets were readily annotated by known gene signatures (FIGS. 1B-1C)^{40,47-49}. While earlier scRNA-seq studies in PDAC did not fully capture the stromal milieu with marked underrepresentation of cancer-associated fibroblasts (CAFs)⁵⁰⁻⁵², they are well-represented in all the samples (FIGS. 1B-1C, FIG. 5).

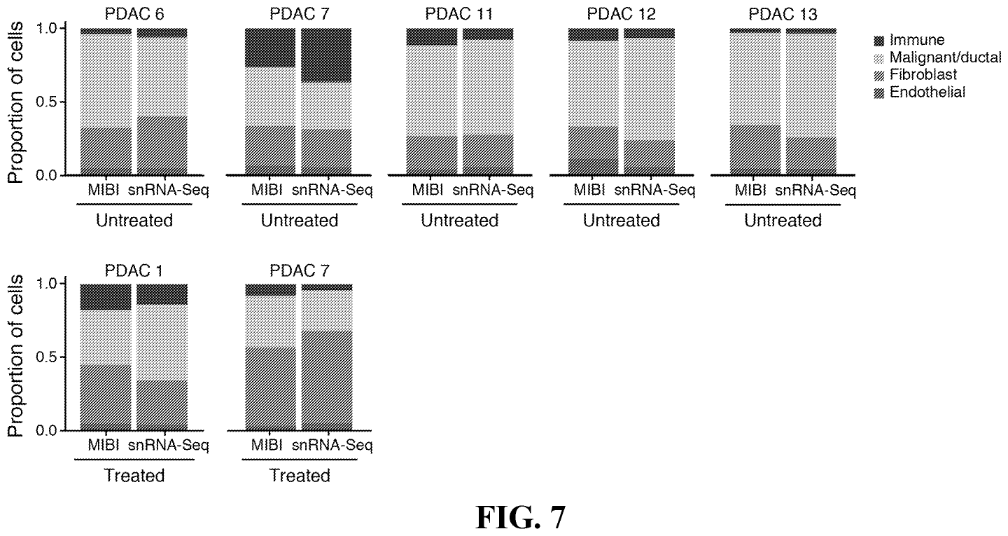

To further assess the method captured representative cell type proportions, it was compared to estimates from Multiplexed Ion Beam Imaging (MIBI), using a 27-plex epithelial oncology panel on formalin-fixed paraffin-embedded (FFPE) sections derived from tumor specimens in a subset of seven individuals (FIG. 1A, Methods)^{53,54}. This confirmed that snRNA-seq captures a representative distribution of the major cell types present in PDAC, both in aggregate across all tumors (FIG. 1D), and individually (FIG. 7).

### Compartment-Specific Remodeling of Cell Composition and Intrinsic Programs Following Neoadjuvant Treatment has Implications for Anti-Tumor Immunity

The snRNA-seq cell type proportions for the treated and untreated cohorts were compared (FIG. 2A, FIG. 8). As expected, the proportion of malignant cells was significantly lower in the treated cohort, which were confirmed by histology (p<5×10⁻⁵, Fisher's exact test). In contrast, within the non-malignant compartment, there were proportionally more acinar cells (p<5×10⁻⁵, Fisher's exact test), endocrine cells (p<5×10⁻⁵), and Schwann cells (p<5×10⁻⁵) associated with neoadjuvant CRT (FIG. 2A; FIG. 8). This was consistent with the higher density of regenerating pancreatic tissue and the previously described resistance of some of these cell types to cytotoxic therapy⁵⁵. Interestingly, after removal of malignant cells, there were proportionally fewer fibroblasts in the CRT cohort (relative to immune and other cells) compared to the untreated group (FIG. 8), suggesting that the histologically-apparent enhanced desmoplastic reaction after treatment may not be dependent on CAF proliferation but rather an enrichment in CAF phenotypes that contribute to the desmoplastic reaction (e.g., myofibroblasts)⁵⁶. There were substantially more Schwann cells in the CRT cohort despite their known radiosensitivity⁵⁷, which could be due to an active repopulation or recruitment of Schwann cells and their associated nerves to the site of treatment-induced injury. Notably, also detected was a population of regenerating acinar cells (REG3A^{high};REG3G^{high};SYCN^{high}) in the treated specimens (FIG. 1C), which have been associated with acinar-to-ductal metaplasia and pancreatic intraepithelial neoplasia^{40,48,58,59}.

The immune compartment from treated tumors was distinct from that of the untreated tumors: there were significantly lower proportions of B cells (Fisher's exact p<5×10⁻⁵), plasma cells (p<5×10⁻⁵), and regulatory T cells (p<5×10⁻⁵) but higher proportions of CD4+ T cells (p=1×10⁻⁴) and macrophages (p<5×10⁻⁵) (FIG. 2A; FIG. 8). Also observed was a marked difference in dendritic cell (DC) subsets. First, conventional type 2 dendritic cells (cDC2), plasmacytoid dendritic cells (pDC) and mature regulatory dendritic cells (mregDCs)⁶⁰⁻⁶², which can suppress anti-tumor immunity in certain contexts, were prominent DC subtypes in treatment-naive samples, but absent from CRT specimens (FIG. 2A)⁶³. In contrast, conventional type 1 dendritic cells (cDC1), which activate cytotoxic lymphocytes critical for anti-tumor immunity, was the only DC subset detected in post-CRT tumors (FIG. 2A). Second, receptor-ligand analysis inferred enhanced interactions between CD40LG⁺ CD4⁺ T cells and CD40⁺ DCs in the CRT cohort (FIG. 2B; Methods). These results are consistent with pre-clinical and clinical reports that CRT can induce immunogenic cell death, which increases tumor antigen availability, and stimulates the production of type I interferons, in turn activating DCs away from regulatory/suppressive cDC2, pDC and mregDC states towards improved capacity for cross-presentation¹⁷⁻²²,. Moreover, combinations of agonistic CD40 antibodies with chemotherapy and PD-1 inhibition has substantial benefit in some patient groups⁶⁴.

Intrinsic gene expression levels in immune cells differed as a function of treatment status (Methods), even in subsets whose proportions were comparable. For example, following CRT, CD8+T lymphocytes expressed markers of altered differentiation (e.g., SLAMF6, CD69, STAT4, IL7R, shift from ITGAE to ITGA4) and TCR signaling (e.g., ITK and FYN) (FIG. 2D; FIG. 9A; Table 2.1-2.6). While most well-established immune checkpoint receptors were not differentially expressed, ENTPD1 (CD39) was marginally lower in the treated group (Table 2.1-2.6). In contrast, both inhibitory (CD96) and activating (CD226) members of the TIGIT/CD155 immune checkpoint family⁶⁵ were higher in the post-treatment context. In macrophages, there was a treatment-associated lower expression of genes for the uncommitted state MO^{47,66} (e.g., MARCO, AQP9, CYP27A1, NCF2; FIG. 2D; FIG. 9B, Table 2.1-2.6), and higher expression of macrophage polarization markers representing both the classical “pro-inflammatory” state M1 (CD86, TNFSF8, TNFRSFIB, IFI44L, LILRB2) and alternative “tissue repair” program M2 (CD163, MRC1, TGFB1, TGFB1, TGFBR2, SIGLEC1, MS4A6A, FES)^{47,67,69}. CRT was also associated with higher expression of MHC class II (MHC-II) (CD74, HLA-DPA1, HLA-DPB1) and TGF-β pathway genes in macrophages (FIG. 2D); FIG. 9B). Concomitantly, the lower expression of the TGF-β pathway in CAFs from treated tumors (FIG. 15A) suggests a potential myeloid-specific immunosuppressive mechanism and may partly explain the efficacy of the TGF-03 modulator, losartan, in combination with neoadjuvant CRT^{6,70}.

TABLE 2.1
Malignant by Residual

| Gene | Score | Pval | Log₂FC | Gene | Score | Pval | Log₂FC | Gene | Score | Pval | Log₂FC | Gene | Score | Pval | Log₂FC |
|---|---|---|---|---|---|---|---|---|---|---|---|---|---|---|---|
| TMSB10 | 13.91 | 1.85E−42 | 2.5 | TMC4 | 4.04 | 1.41E−04 | 1 | ONECUT3 | 2.47 | 2.92E−02 | 1.82 | EDIL3 | 1.5 | 2.53E−01 | 1.84 |
| TJP2 | 13.72 | 2.21E−41 | 3.59 | ST3GAL3 | 3.97 | 1.87E−04 | 2.94 | GREM1 | 2.45 | 3.02E−02 | 3.36 | ST6GALNAC6 | 1.49 | 2.55E−01 | 6.72 |
| PMEPA1 | 13.06 | 1.27E−37 | 1.59 | SLC35A3 | 3.94 | 2.08E−04 | 2.64 | BCAT1 | 2.44 | 3.14E−02 | 2.15 | LHB | 1.49 | 2.56E−01 | 3.92 |
| ZNF83 | 12.74 | 6.62E−36 | 1.69 | FTL | 3.93 | 2.19E−04 | 1.56 | MIR4435-2HG | 2.44 | 3.14E−02 | 0.32 | GCSH | 1.49 | 2.59E−01 | 1.91 |
| LINC00511 | 12.4 | 4.13E−34 | 1.4 | WNT5B | 3.9 | 2.43E−04 | 2.4 | HIST2H2BE | 2.42 | 3.31E−02 | 2.19 | NBAT1 | 1.46 | 2.69E−01 | 3.26 |
| KRT19 | 10.45 | 1.21E−24 | 0.58 | CLDN1 | 3.89 | 2.52E−04 | 1.06 | PRSS21 | 2.41 | 3.37E−02 | 3.59 | EEF1A2 | 1.46 | 2.70E−01 | 3.14 |
| DDIT4 | 10.27 | 7.74E−24 | 1.77 | TNC | 3.88 | 2.72E−04 | 2.8 | MMP3 | 2.38 | 3.65E−02 | 2.19 | NDUFA3 | 1.44 | 2.82E−01 | 2.51 |
| UBC | 9.82 | 6.43E−22 | 1.36 | ACTG2 | 3.87 | 2.81E−04 | 5.38 | TGFBI | 2.35 | 3.94E−02 | 2.54 | CERCAM | 1.43 | 2.83E−01 | 2.34 |
| KRT17 | 9.54 | 9.81E−21 | 2.33 | RPS15 | 3.85 | 3.05E−04 | 1.75 | CST6 | 2.35 | 3.95E−02 | 2.27 | PIGM | 1.42 | 2.91E−01 | 3.58 |
| ZNF804A | 9.52 | 1.16E−20 | 6.1 | ID1 | 3.83 | 3.19E−04 | 1.75 | DPYSL3 | 2.31 | 4.42E−02 | 2.21 | ERCC5 | 1.42 | 2.92E−01 | 2.56 |
| COL1A2 | 9.19 | 2.36E−19 | 2.52 | HSPB1 | 3.81 | 3.58E−04 | 1.23 | SLC35F3 | 2.31 | 4.37E−02 | 0.91 | CARMN | 1.4 | 2.98E−01 | 2.46 |
| MT2A | 9.18 | 2.77E−19 | 3.07 | NF1 | 3.8 | 3.70E−04 | −0.05 | NTF4 | 2.3 | 4.50E−02 | 3.62 | WWTR1 | 1.39 | 3.07E−01 | 0.11 |
| USP39 | 8.74 | 1.27E−17 | 1.94 | LINC01481 | 3.74 | 4.68E−04 | 3.11 | C11orf54 | 2.3 | 4.46E−02 | 2.27 | HLA-C | 1.36 | 3.20E−01 | 1.46 |
| COL1A1 | 8.55 | 6.87E−17 | 2.24 | COL12A1 | 3.72 | 5.01E−04 | 2.15 | SULF1 | 2.28 | 4.76E−02 | 2.01 | RFX8 | 1.35 | 3.25E−01 | 2.96 |
| FN1 | 8.28 | 6.42E−16 | 2.55 | ANO1 | 3.71 | 5.09E−04 | 1.45 | MMP11 | 2.26 | 4.91E−02 | 4.1 | EDF1 | 1.35 | 3.28E−01 | 1.21 |
| AC159540.1 | 7.95 | 8.54E−15 | 3.91 | COMMD7 | 3.7 | 5.36E−04 | 3.02 | CXCL14 | 2.26 | 5.00E−02 | 2.56 | PMAIP1 | 1.34 | 3.30E−01 | 2.31 |
| RPL28 | 7.95 | 9.01E−15 | 2.26 | PKIA | 3.64 | 6.72E−04 | 3.47 | BCAS3 | 2.26 | 4.94E−02 | −0.03 | C16orf74 | 1.34 | 3.30E−01 | 1.27 |
| HMGA2 | 7.87 | 1.68E−14 | 2.86 | OR2A1-AS1 | 3.6 | 7.91E−04 | 2.13 | RNASE10 | 2.25 | 5.09E−02 | 1.84 | AC008074.3 | 1.33 | 3.39E−01 | 2.03 |
| LINGO1 | 7.87 | 1.70E−14 | 1.83 | FXN | 3.58 | 8.52E−04 | 4.9 | HIST1H2BB | 2.24 | 5.21E−02 | 1.87 | EFCAB3 | 1.33 | 3.36E−01 | 1.7 |
| ACTB | 7.78 | 3.21E−14 | 1.01 | ARHGAP11B | 3.56 | 9.25E−04 | 4.89 | C9orf16 | 2.23 | 5.41E−02 | 2.25 | ITGA3 | 1.33 | 3.38E−01 | 0.03 |
| COL3A1 | 7.71 | 5.78E−14 | 2.43 | NAP3B | 3.54 | 9.90E−04 | 1.91 | RBMS3 | 2.21 | 5.56E−02 | 1.65 | SYNM | 1.32 | 3.44E−01 | 3.01 |
| KRT7 | 7.68 | 6.99E−14 | 1.4 | ANXA8L1 | 3.49 | 1.17E−03 | 2.01 | COL4A2 | 2.21 | 5.66E−02 | 1.41 | MKL1 | 1.32 | 3.44E−01 | 0.13 |
| FKBP10 | 7.6 | 1.33E−13 | 3.11 | LRRFIP1 | 3.49 | 1.19E−03 | −0.05 | EEPD1 | 2.2 | 5.68E−02 | 0.44 | SMOC2 | 1.31 | 3.50E−01 | 2.08 |
| IGF2BP2 | 7.58 | 1.53E−13 | 0.39 | TNS4 | 3.48 | 1.24E−03 | 1.61 | COMP | 2.17 | 6.19E−02 | 2.75 | CCND2 | 1.31 | 3.50E−01 | 1.54 |
| PLEC | 7.53 | 2.22E−13 | 0.85 | TEX41 | 3.44 | 1.40E−03 | 3.07 | RPS17 | 2.17 | 6.18E−02 | 1.68 | TOX2 | 1.3 | 3.55E−01 | 3.3 |
| ZC3H11A | 7.48 | 3.12E−13 | 2.79 | MT1E | 3.43 | 1.46E−03 | 2.57 | ISG15 | 2.17 | 6.19E−02 | 1.59 | EMILIN1 | 1.3 | 3.52E−01 | 2.13 |
| RANBP17 | 7.02 | 8.89E−12 | 1.05 | THBS2 | 3.42 | 1.54E−03 | 2.8 | MBNL1 | 2.17 | 6.18E−02 | −0.17 | AP002884.2 | 1.3 | 3.52E−01 | 2.03 |
| KCTD7 | 6.77 | 4.80E−11 | 3.72 | MYH11 | 3.39 | 1.70E−03 | 2.3 | CD59 | 2.16 | 6.39E−02 | 1.4 | ANKRD1 | 1.28 | 3.65E−01 | 2.76 |
| FOXP2 | 6.76 | 5.41E−11 | 2.22 | SPARC | 3.37 | 1.82E−03 | 2.08 | CDH4 | 2.15 | 6.54E−02 | 2.7 | PRC1 | 1.27 | 3.73E−01 | 3.61 |
| ALDOA | 6.75 | 5.70E−11 | 2.76 | RPL36 | 3.35 | 1.93E−03 | 1.43 | MFSD14A | 2.14 | 6.64E−02 | 2.11 | KRT8 | 1.27 | 3.72E−01 | 0.26 |
| PARL | 6.59 | 1.61E−10 | 4.47 | HDAC9 | 3.34 | 2.00E−03 | 0.63 | CLDN11 | 2.13 | 6.75E−02 | 2.95 | IFITM10 | 1.26 | 3.78E−01 | 3.16 |
| AC019117.2 | 6.39 | 6.12E−10 | 4.44 | RPS11 | 3.33 | 2.10E−03 | 1.51 | GGPS1 | 2.11 | 7.16E−02 | 2.77 | APOBEC2 | 1.25 | 3.83E−01 | 3.04 |
| MET | 6.39 | 5.80E−10 | 0.66 | LVRN | 3.3 | 2.32E−03 | 5.77 | RPL41 | 2.1 | 7.21E−02 | 1.26 | AC010967.2 | 1.25 | 3.86E−01 | 2.05 |
| LINC01322 | 6.32 | 9.11E−10 | 2.17 | FGD4 | 3.3 | 2.26E−03 | −0.08 | SORCS2 | 2.08 | 7.67E−02 | 2.62 | MRC2 | 1.25 | 3.86E−01 | 1.99 |
| RPL13A | 6.28 | 1.19E−09 | 1.73 | TIMP3 | 3.29 | 2.35E−03 | 2.06 | KDM4A | 2.08 | 7.66E−02 | 2.3 | AHNAK2 | 1.25 | 3.85E−01 | 1.17 |
| DCBLD2 | 6.25 | 1.47E−09 | 1.89 | GAPDH | 3.29 | 2.41E−03 | 1.42 | IGSF1 | 2.07 | 7.87E−02 | 2.83 | BCL2L1 | 1.25 | 3.84E−01 | −0.01 |
| ZFP36L1 | 6.11 | 3.45E−09 | 0.98 | RPL8 | 3.28 | 2.43E−03 | 1.53 | AGGF1 | 2.07 | 7.81E−02 | 2.73 | CHST11 | 1.22 | 4.00E−01 | 1.24 |
| S100A6 | 6.08 | 4.10E−09 | 0.98 | TP63 | 3.27 | 2.52E−03 | 2.04 | RPLP2 | 2.05 | 8.28E−02 | 1.22 | ABCF2 | 1.21 | 4.11E−01 | 2.57 |
| FTH1 | 6.06 | 4.72E−09 | 1.51 | LDLRAD4 | 3.24 | 2.86E−03 | 0.86 | PABPC1 | 2.03 | 8.67E−02 | 0.26 | BGN | 1.21 | 4.11E−01 | 2.09 |
| MYOF | 5.99 | 7.02E−09 | 0.24 | KCNQ5 | 3.22 | 2.97E−03 | 2.88 | FRMD6 | 2.02 | 8.71E−02 | 1.45 | C4orf36 | 1.21 | 4.11E−01 | 1.59 |
| RPL13 | 5.98 | 7.72E−09 | 1.1 | KLF12 | 3.21 | 3.11E−03 | 1.63 | CMTM3 | 2.01 | 9.02E−02 | 2.78 | LCP1 | 1.2 | 4.15E−01 | 2.45 |
| DES | 5.8 | 2.15E−08 | 6.33 | ITGB4 | 3.21 | 3.13E−03 | 0.46 | FSTL3 | 1.98 | 9.62E−02 | 1.72 | HES4 | 1.2 | 4.15E−01 | 1.66 |
| IFNAR2 | 5.75 | 2.86E−08 | 2.96 | CLN8 | 3.2 | 3.22E−03 | 2.42 | CHN2 | 1.98 | 9.63E−02 | 0.71 | AGAP1 | 1.2 | 4.16E−01 | −0.28 |
| TMSB4X | 5.75 | 2.93E−08 | 1.06 | CASC15 | 3.16 | 3.71E−03 | 2.12 | FAM49B | 1.97 | 9.88E−02 | −0.01 | GJA1 | 1.19 | 4.23E−01 | 2.32 |
| AC026167.1 | 5.73 | 3.33E−08 | 3.05 | FLNA | 3.16 | 3.64E−03 | 1.37 | SPATS2L | 1.97 | 9.76E−02 | −0.14 | DGKG | 1.18 | 4.31E−01 | 1.51 |
| FNDC3B | 5.68 | 4.36E−08 | 0.1 | EPB42 | 3.07 | 4.94E−03 | 1.63 | CCDC85B | 1.96 | 1.00E−01 | 2.14 | MIR646HG | 1.17 | 4.36E−01 | 2.11 |
| AKAP12 | 5.65 | 5.29E−08 | 2.53 | S100A2 | 2.99 | 6.42E−03 | 3.77 | CCND1 | 1.95 | 1.02E−01 | 1.03 | LINC00923 | 1.17 | 4.34E−01 | 1.32 |
| ZNF544 | 5.6 | 6.97E−08 | 3.39 | CSNK1E | 2.95 | 7.18E−03 | 1.57 | GPC1 | 1.93 | 1.06E−01 | 2.24 | VIM | 1.17 | 4.34E−01 | 1.18 |
| SLC26A3 | 5.59 | 7.27E−08 | 1.59 | RPS9 | 2.94 | 7.36E−03 | 1.19 | CRIP2 | 1.9 | 1.14E−01 | 1.32 | KRT6A | 1.15 | 4.49E−01 | 3.69 |
| LGALS1 | 5.58 | 7.57E−08 | 3.7 | SLCO4A1 | 2.92 | 8.06E−03 | 2.43 | ITGB5 | 1.89 | 1.18E−01 | 0.76 | CLN5 | 1.15 | 4.46E−01 | 2.37 |
| SOD2 | 5.54 | 9.54E−08 | 2.35 | POSTN | 2.87 | 9.40E−03 | 2.53 | SLIT2 | 1.86 | 1.23E−01 | 1.62 | MYO3B | 1.14 | 4.55E−01 | 1.59 |
| MALAT1 | 5.47 | 1.41E−07 | 0.24 | TLL1 | 2.85 | 9.71E−03 | 3.74 | TNNT1 | 1.84 | 1.31E−01 | 3.56 | SNAI2 | 1.13 | 4.59E−01 | 2.94 |
| VCAN | 5.37 | 2.47E−07 | 1.76 | MSC-AS1 | 2.85 | 9.75E−03 | 2.35 | FGF19 | 1.84 | 1.29E−01 | 2.98 | TMEM47 | 1.12 | 4.65E−01 | 1.74 |
| ZNF665 | 5.36 | 2.58E−07 | 2.77 | COL11A1 | 2.82 | 1.07E−02 | 2.77 | MED17 | 1.84 | 1.29E−01 | 2.72 | ATP7A | 1.11 | 4.78E−01 | 1.76 |
| ARHGEF10 | 5.34 | 2.84E−07 | 3.52 | FKBPS | 2.82 | 1.07E−02 | 0.45 | FGD6 | 1.83 | 1.31E−01 | 0.26 | FIGN | 1.11 | 4.73E−01 | 1.02 |
| IGFBP3 | 5.26 | 4.45E−07 | 3 | COL5A1 | 2.79 | 1.19E−02 | 2.33 | VMP1 | 1.83 | 1.32E−01 | −0.16 | SPG7 | 1.11 | 4.73E−01 | 0.37 |
| PLAT | 5.25 | 4.68E−07 | 2.04 | DEPDC5 | 2.79 | 1.19E−02 | 2.21 | FBN1 | 1.82 | 1.34E−01 | 1.82 | TRIO | 1.11 | 4.76E−01 | −0.26 |
| TANC2 | 5.17 | 7.02E−07 | 0.59 | BMS1P14 | 2.78 | 1.23E−02 | 1.34 | ARL4D | 1.81 | 1.38E−01 | 4.8 | SGIP1 | 1.1 | 4.81E−01 | 1.85 |
| LRP4 | 5.13 | 8.75E−07 | 2.2 | LY6K | 2.76 | 1.30E−02 | 26.59 | PTMA | 1.81 | 1.38E−01 | 0.96 | IL10RB | 1.1 | 4.84E−01 | 1.71 |
| RPL35 | 5.07 | 1.15E−06 | 1.8 | TBCEL | 2.76 | 1.30E−02 | 4.82 | PDE4C | 1.8 | 1.41E−01 | 2.63 | INHBA | 1.1 | 4.80E−01 | 1.65 |
| ARPC1A | 5.06 | 1.25E−06 | 2.71 | C12orf56 | 2.76 | 1.29E−02 | 2.85 | SOSTDC1 | 1.79 | 1.44E−01 | 7 | ITGBL1 | 1.1 | 4.79E−01 | 1.23 |
| RAD50 | 5.02 | 1.53E−06 | 2.49 | IGFBP6 | 2.73 | 1.41E−02 | 3.16 | ZBTB20 | 1.77 | 1.51E−01 | −0.25 | AP3S1 | 1.1 | 4.85E−01 | 1.03 |
| AEBP1 | 4.86 | 3.43E−06 | 2.74 | PLAU | 2.73 | 1.42E−02 | 1.67 | SKIL | 1.76 | 1.52E−01 | 0.4 | AMT | 1.09 | 4.92E−01 | 2.39 |
| CD109 | 4.76 | 5.41E−06 | 1.91 | IGFBP5 | 2.64 | 1.82E−02 | 3.01 | GATA5 | 1.74 | 1.59E−01 | 25.69 | ST8SIA6-AS1 | 1.08 | 4.98E−01 | 4.89 |
| ANXA1 | 4.55 | 1.46E−05 | 0.69 | PDLIM7 | 2.64 | 1.81E−02 | 1.2 | LIMD2 | 1.73 | 1.64E−01 | 3.09 | THY1 | 1.08 | 4.97E−01 | 3.48 |
| TCF7L1 | 4.53 | 1.63E−05 | 3.07 | RPLP1 | 2.62 | 1.94E−02 | 1.01 | THBS1 | 1.73 | 1.62E−01 | 1.58 | ADH1C | 1.08 | 4.95E−01 | 2.95 |
| RPL10 | 4.51 | 1.80E−05 | 1.03 | SLC7A5 | 2.61 | 2.00E−02 | 2.59 | PPDPF | 1.73 | 1.61E−01 | 1.55 | ZFP57 | 1.07 | 5.06E−01 | 24.75 |
| CAMK1D | 4.4 | 2.90E−05 | 1.43 | PSD3 | 2.59 | 2.09E−02 | 0.49 | IGFBP7 | 1.73 | 1.63E−01 | 0.87 | HTRA3 | 1.05 | 5.19E−01 | 4.02 |
| MYH14 | 4.3 | 4.59E−05 | 0.57 | ZNF37A | 2.58 | 2.17E−02 | 2.8 | KRT5 | 1.68 | 1.79E−01 | 3.95 | PTGES | 1.05 | 5.18E−01 | 2.5 |
| C19orf33 | 4.29 | 4.78E−05 | 2.26 | NUPR1 | 2.57 | 2.21E−02 | 2.1 | NMUR2 | 1.67 | 1.82E−01 | 1.73 | TPX2 | 1.05 | 5.17E−01 | 1.81 |
| COL6A2 | 4.25 | 5.85E−05 | 2.64 | RPS19 | 2.57 | 2.23E−02 | 1.07 | SCO2 | 1.65 | 1.90E−01 | 2.53 | RPL38 | 1.05 | 5.17E−01 | 1.18 |
| TGM2 | 4.25 | 5.64E−05 | 0.77 | SERPINE1 | 2.56 | 2.29E−02 | 1.53 | COL7A1 | 1.61 | 2.07E−01 | 1.72 | AC005253.2 | 1.04 | 5.26E−01 | 24.81 |
| CRHR1 | 4.24 | 5.98E−05 | 3.33 | COL8A1 | 2.55 | 2.32E−02 | 2.52 | RAB34 | 1.6 | 2.08E−01 | 2.53 | TRIM29 | 1.04 | 5.23E−01 | 1.07 |
| CNTNAP3 | 4.21 | 6.78E−05 | 3.36 | PTK2 | 2.54 | 2.38E−02 | −0.14 | RPL18 | 1.59 | 2.12E−01 | 1.59 | RNF181 | 1.03 | 5.31E−01 | 1.65 |
| SLC33A1 | 4.18 | 7.61E−05 | 4.77 | HSD17B3 | 2.51 | 2.58E−02 | 3.09 | RORA | 1.58 | 2.19E−01 | 0.36 | UNC5B-AS1 | 1.02 | 5.36E−01 | 1.91 |
| FSCN1 | 4.11 | 1.02E−04 | 3.59 | SLCO1B1 | 2.5 | 2.68E−02 | 3.42 | ATG9A | 1.56 | 2.24E−01 | 2.04 | SLC2A3 | 1.02 | 5.39E−01 | 1.45 |
| TAGLN | 4.11 | 1.05E−04 | 2.9 | PKM | 2.5 | 2.67E−02 | 0.46 | IGF2 | 1.54 | 2.32E−01 | 3.01 | LY6E | 1.01 | 5.47E−01 | 1.41 |
| MYO16-AS1 | 4.06 | 1.29E−04 | 4.53 | MMP2 | 2.47 | 2.87E−02 | 2.73 | C16orf46 | 1.51 | 2.50E−01 | 1.88 | | | | |
| CALD1 | 4.06 | 1.30E−04 | 1.71 | FHOD3 | 2.47 | 2.93E−02 | 2.04 | COX6B2 | 1.5 | 2.54E−01 | 2.61 | | | | |

Abbreviations: FC = fold change;

TABLE 2.2
Malignant no BRCA by treatment

| Gene | Score | Pval | Log₂FC | Gene | Score | Pval | Log₂FC | Gene | Score | Pval | Log₂FC | Gene | Score | Pval | Log₂FC |
|---|---|---|---|---|---|---|---|---|---|---|---|---|---|---|---|
| ZNF83 | 41.41 | 0.00E+00 | 1.97 | EDARADD | 6.17 | 6.62E−09 | 1.26 | UBB | 3.92 | 4.92E−04 | 0.4 | PRELID3A | 2.74 | 2.47E−02 | 0.86 |
| TJP2 | 39.73 | 0.00E+00 | 3.24 | IGF2BP2-AS1 | 6.17 | 6.48E−09 | 0.89 | POLR2B | 3.92 | 5.04E−04 | 0.25 | FAM71F2 | 2.74 | 2.51E−02 | 0.72 |
| USP39 | 36.76 | 1.84E−292 | 2.11 | MAP3K11 | 6.17 | 6.76E−09 | 0.79 | ASIC2 | 3.91 | 5.05E−04 | 1.25 | YIPF3 | 2.74 | 2.48E−02 | 0.41 |
| HDAC9 | 34.94 | 3.63E−264 | 2.01 | PRDX5 | 6.17 | 6.67E−09 | 0.78 | DYRK1B | 3.91 | 5.05E−04 | 0.84 | NARFL | 2.74 | 2.48E−02 | 0.33 |
| TGM2 | 32.36 | 1.28E−226 | 1.77 | SPRY1 | 6.16 | 7.08E−09 | 1.1 | ATP6AP2 | 3.91 | 5.10E−04 | 0.61 | ANKRD49 | 2.74 | 2.45E−02 | 0.3 |
| TMSB10 | 32.26 | 2.99E−225 | 1.58 | SLC35G2 | 6.16 | 6.81E−09 | 0.48 | CERK | 3.91 | 5.21E−04 | 0.56 | LPIN2 | 2.74 | 2.50E−02 | 0.19 |
| HLA-B | 31.08 | 4.29E−209 | 1.88 | GRIK2 | 6.15 | 7.63E−09 | 1.35 | PLAG1 | 3.91 | 5.18E−04 | 0.27 | SF3B6 | 2.74 | 2.45E−02 | 0.18 |
| ZNF804A | 28.64 | 1.83E−177 | 6.01 | CLTCL1 | 6.15 | 7.54E−09 | 1.12 | ITM2C | 3.9 | 5.35E−04 | 1.16 | RMI2 | 2.73 | 2.55E−02 | 0.89 |
| GABRP | 28.27 | 7.23E−173 | 1.77 | TMEM106A | 6.15 | 7.60E−09 | 1.06 | MFSD3 | 3.9 | 5.32E−04 | 1.06 | THAP7 | 2.73 | 2.58E−02 | 0.66 |
| MT2A | 28.03 | 5.95E−170 | 2.4 | LINC01091 | 6.15 | 7.31E−09 | 0.68 | CHST12 | 3.9 | 5.36E−04 | 0.76 | TUBA1A | 2.73 | 2.56E−02 | 0.51 |
| POU5F1 | 26.86 | 4.07E−156 | 1.75 | MLEC | 6.15 | 7.31E−09 | 0.67 | ORAI2 | 3.9 | 5.39E−04 | 0.69 | MIR762HG | 2.73 | 2.55E−02 | 0.5 |
| RPL28 | 26.6 | 3.55E−153 | 1.79 | C9orf3 | 6.15 | 7.59E−09 | 0.18 | ASB7 | 3.9 | 5.38E−04 | 0.4 | TRAFD1 | 2.73 | 2.52E−02 | 0.41 |
| C19orf33 | 26.41 | 5.36E−151 | 2.68 | SLC26A8 | 6.14 | 7.97E−09 | 1.16 | TRIOBP | 3.9 | 5.38E−04 | 0.29 | MCEE | 2.73 | 2.53E−02 | 0.33 |
| XIST | 26.34 | 3.93E−150 | 3.21 | ZNF600 | 6.14 | 8.04E−09 | 0.72 | COL24A1 | 3.89 | 5.65E−04 | 1.27 | HMOX2 | 2.73 | 2.53E−02 | 0.28 |
| FKBP10 | 25.98 | 3.85E−146 | 2.86 | WNT7B | 6.13 | 8.40E−09 | 1.3 | FUCA1 | 3.89 | 5.54E−04 | 1.07 | TRPC1 | 2.73 | 2.56E−02 | 0.21 |
| AC159540.1 | 25.68 | 9.84E−143 | 4.48 | UGT2B7 | 6.12 | 8.77E−09 | 0.31 | TMEM147 | 3.89 | 5.47E−04 | 0.84 | DSTN | 2.73 | 2.53E−02 | 0.19 |
| ACTB | 25.44 | 4.71E−140 | 1.14 | FCGBP | 6.11 | 9.44E−09 | 2.26 | BIK | 3.89 | 5.59E−04 | 0.67 | METRNL | 2.72 | 2.62E−02 | 0.76 |
| ACTN4 | 25.43 | 4.71E−140 | 0.88 | NDUFA1 | 6.11 | 9.56E−09 | 1.34 | RAB35 | 3.89 | 5.47E−04 | 0.64 | SERTAD1 | 2.72 | 2.60E−02 | 0.65 |
| FTH1 | 25.26 | 3.68E−138 | 1.46 | TMEM263 | 6.11 | 9.70E−09 | 0.56 | C15orf48 | 3.89 | 5.56E−04 | 0.6 | STXBP5-AS1 | 2.72 | 2.65E−02 | 0.63 |
| DDIT4 | 25.04 | 8.15E−136 | 1.14 | COPB2 | 6.11 | 9.45E−09 | 0.45 | BANK1 | 3.89 | 5.46E−04 | 0.25 | ITM2B | 2.72 | 2.60E−02 | 0.35 |
| LINC01322 | 25.01 | 1.90E−135 | 1.88 | NDUFA12 | 6.1 | 9.79E−09 | 0.89 | MSN | 3.89 | 5.53E−04 | 0.21 | FAM81A | 2.72 | 2.59E−02 | 0.31 |
| LRP4 | 24.85 | 1.01E−133 | 1.9 | IFT81 | 6.1 | 9.85E−09 | 0.67 | ROBO1 | 3.89 | 5.60E−04 | 0.13 | GARNL3 | 2.72 | 2.59E−02 | 0.28 |
| PMEPA1 | 24.81 | 2.40E−133 | 0.94 | AP5Z1 | 6.1 | 1.03E−08 | 0.65 | ZDHHC12 | 3.88 | 5.82E−04 | 1.14 | CHSY3 | 2.72 | 2.63E−02 | 0.26 |
| KRT19 | 24.74 | 1.54E−132 | 0.6 | ANKS1A | 6.1 | 9.97E−09 | 0.35 | ZNF347 | 3.88 | 5.67E−04 | 0.94 | ZNF587 | 2.72 | 2.64E−02 | 0.21 |
| ZFAND3 | 24.45 | 1.66E−129 | 0.62 | SORCS2 | 6.09 | 1.05E−08 | 1.12 | ST6GALNAC2 | 3.88 | 5.81E−04 | 0.8 | AHCYL1 | 2.72 | 2.64E−02 | 0.2 |
| ZC3H11A | 23.75 | 3.61E−122 | 2.74 | EIF2AK2 | 6.09 | 1.09E−08 | 0.41 | ETFB | 3.88 | 5.78E−04 | 0.62 | KCNQ10T1 | 2.72 | 2.62E−02 | 0.16 |
| RPL13A | 23.37 | 2.43E−118 | 1.45 | ATG4B | 6.09 | 1.08E−08 | 0.35 | CD276 | 3.88 | 5.70E−04 | 0.58 | NR6A1 | 2.72 | 2.61E−02 | 0.13 |
| ALDOA | 23.19 | 1.74E−116 | 3.42 | TRERF1 | 6.08 | 1.16E−08 | 0.33 | NOC2L | 3.88 | 5.81E−04 | 0.46 | ZFP57 | 2.71 | 2.72E−02 | 3.39 |
| RPL35 | 22.35 | 3.18E−108 | 1.6 | DKK3 | 6.07 | 1.20E−08 | 1.95 | LRRC14 | 3.88 | 5.82E−04 | 0.46 | CCDC178 | 2.71 | 2.69E−02 | 1.5 |
| RPLP1 | 22.28 | 1.33E−107 | 1.58 | SKIV2L | 6.07 | 1.18E−08 | 0.94 | TBC1D16 | 3.88 | 5.81E−04 | 0.38 | CDH18 | 2.71 | 2.66E−02 | 1.27 |
| CCND1 | 22.2 | 8.12E−107 | 1.58 | ERCC5 | 6.06 | 1.27E−08 | 2.82 | MMRN2 | 3.87 | 6.08E−04 | 1.5 | PCDH19 | 2.71 | 2.70E−02 | 1.17 |
| SYT1 | 22.07 | 1.43E−105 | 3.58 | AKAP12 | 6.06 | 1.26E−08 | 0.56 | AC007099.1 | 3.87 | 5.91E−04 | 1.05 | BBOF1 | 2.71 | 2.65E−02 | 0.53 |
| RPL13 | 22.07 | 1.27E−105 | 1.08 | PDLIM1 | 6.06 | 1.32E−08 | 0.38 | RCAN1 | 3.87 | 6.03E−04 | 0.89 | PNPLA6 | 2.71 | 2.69E−02 | 0.25 |
| CCND3 | 21.78 | 8.17E−103 | 0.98 | FAM102A | 6.06 | 1.32E−08 | 0.38 | DGKI | 3.87 | 6.03E−04 | 0.79 | NAV2-AS3 | 2.7 | 2.76E−02 | 1.95 |
| RPLP2 | 21.68 | 6.95E−102 | 1.46 | FAM184B | 6.05 | 1.32E−08 | 1.05 | ACOT7 | 3.87 | 6.03E−04 | 0.62 | NOX01 | 2.7 | 2.79E−02 | 0.99 |
| CD59 | 21.38 | 4.10E−99 | 1.79 | MPHOSPH9 | 6.05 | 1.35E−08 | 0.63 | DDA1 | 3.87 | 6.02E−04 | 0.5 | TMEM139 | 2.7 | 2.76E−02 | 0.93 |
| TMSB4X | 21.29 | 2.45E−98 | 1.02 | LRRC69 | 6.05 | 1.38E−08 | 0.61 | SUGCT | 3.87 | 5.93E−04 | 0.37 | FADS2 | 2.7 | 2.79E−02 | 0.75 |
| RPS19 | 21.25 | 6.66E−98 | 1.41 | TOM1 | 6.05 | 1.35E−08 | 0.54 | CDK10 | 3.87 | 5.89E−04 | 0.24 | FAM3B | 2.7 | 2.77E−02 | 0.53 |
| IFI27 | 21.19 | 2.23E−97 | 1.8 | PSMA7 | 6.04 | 1.43E−08 | 0.66 | CYB5R1 | 3.86 | 6.31E−04 | 0.7 | SUMF2 | 2.7 | 2.75E−02 | 0.18 |
| IFI44L | 21 | 1.12E−95 | 1.96 | ABLIM2 | 6.04 | 1.41E−08 | 0.53 | IL20RA | 3.86 | 6.22E−04 | 0.53 | MBNL1 | 2.7 | 2.75E−02 | 0.03 |
| KRT8 | 20.95 | 3.07E−95 | 1.02 | EIF1 | 6.04 | 1.46E−08 | 0.42 | LAMTOR4 | 3.86 | 6.12E−04 | 0.48 | C3orf33 | 2.69 | 2.85E−02 | 2.33 |
| SOD2 | 20.94 | 3.68E−95 | 2.95 | PLA2G4C | 6.03 | 1.58E−08 | 1.18 | DNPEP | 3.86 | 6.26E−04 | 0.41 | GRM5 | 2.69 | 2.87E−02 | 1.55 |
| CSNKIE | 20.84 | 3.16E−94 | 1.63 | FPGS | 6.03 | 1.55E−08 | 0.5 | COMT | 3.86 | 6.32E−04 | 0.34 | GADD45G | 2.69 | 2.81E−02 | 0.87 |
| ZBTB20 | 20.37 | 4.75E−90 | 0.41 | CCPG1 | 6.02 | 1.64E−08 | 0.47 | IRF2BP2 | 3.86 | 6.12E−04 | 0.32 | PSMB6 | 2.69 | 2.84E−02 | 0.86 |
| LDLRAD4 | 20.21 | 1.32E−88 | 1.11 | CENPB | 6.01 | 1.75E−08 | 1.38 | WDR1 | 3.86 | 6.12E−04 | 0.31 | PALM3 | 2.69 | 2.87E−02 | 0.78 |
| NCALD | 19.84 | 2.16E−85 | 1.54 | GTF2IRD2B | 6.01 | 1.72E−08 | 0.64 | CLEC16A | 3.86 | 6.31E−04 | 0.24 | SRM | 2.69 | 2.81E−02 | 0.66 |
| IFNAR2 | 19.8 | 4.71E−85 | 3.96 | NR2C1 | 6.01 | 1.71E−08 | 0.41 | NCS1 | 3.85 | 6.52E−04 | 0.92 | PALM2 | 2.69 | 2.88E−02 | 0.56 |
| KCTD7 | 19.74 | 1.51E−84 | 3.02 | FTO | 6.01 | 1.68E−08 | 0.26 | GPR132 | 3.85 | 6.58E−04 | 0.6 | GNPTG | 2.69 | 2.81E−02 | 0.37 |
| RAD50 | 19.57 | 3.68E−83 | 3.1 | COA3 | 6 | 1.81E−08 | 1.63 | SRSF9 | 3.85 | 6.53E−04 | 0.54 | FAM3A | 2.69 | 2.85E−02 | 0.36 |
| NAV2 | 19.53 | 8.96E−83 | 1.16 | COL11A1 | 6 | 1.83E−08 | 0.88 | LINC01317 | 3.84 | 6.63E−04 | 1.59 | NCL | 2.69 | 2.88E−02 | 0.24 |
| TRIM29 | 19.35 | 2.87E−81 | 1.38 | TDG | 6 | 1.85E−08 | 0.64 | TUBB8 | 3.84 | 6.63E−04 | 1.25 | USP6 | 2.68 | 2.96E−02 | 1.32 |
| IFI6 | 19.34 | 3.03E−81 | 2.66 | ISOC2 | 5.99 | 1.89E−08 | 1.44 | CCDC146 | 3.84 | 6.77E−04 | 0.25 | RUNX1T1 | 2.68 | 2.96E−02 | 0.74 |
| RPL41 | 19.11 | 2.74E−79 | 1.33 | PLAU | 5.99 | 1.91E−08 | 0.9 | PLAUR | 3.84 | 6.74E−04 | 0.24 | CCDC189 | 2.68 | 2.89E−02 | 0.71 |
| PARL | 19.09 | 3.81E−79 | 3.35 | DENND3 | 5.99 | 1.93E−08 | 0.56 | ICAM2 | 3.83 | 6.91E−04 | 1.13 | THNSL2 | 2.68 | 2.94E−02 | 0.54 |
| EDF1 | 19.08 | 4.84E−79 | 1.32 | LRRC37A3 | 5.99 | 1.94E−08 | 0.35 | SNX17 | 3.83 | 6.99E−04 | 0.92 | MARCKSL1 | 2.68 | 2.90E−02 | 0.47 |
| IL1RAPL2 | 18.98 | 3.33E−78 | 1.79 | LINC01060 | 5.98 | 2.10E−08 | 1.65 | LRFN4 | 3.83 | 7.04E−04 | 0.69 | RWDD1 | 2.68 | 2.95E−02 | 0.47 |
| AC019117.2 | 18.97 | 3.60E−78 | 3.52 | CMTM3 | 5.98 | 2.07E−08 | 1.09 | CEBPG | 3.83 | 6.88E−04 | 0.48 | UBAC1 | 2.68 | 2.93E−02 | 0.33 |
| RPL36 | 18.77 | 1.48E−76 | 1.34 | TRHDE | 5.98 | 2.05E−08 | 0.87 | ZNF529 | 3.83 | 6.91E−04 | 0.47 | PROS1 | 2.68 | 2.90E−02 | 0.29 |
| LY6E | 18.68 | 8.46E−76 | 2.24 | SP100 | 5.98 | 2.08E−08 | 0.29 | FOXK1 | 3.83 | 7.07E−04 | 0.21 | CNKSR1 | 2.68 | 2.89E−02 | 0.27 |
| RPS16 | 18.68 | 8.42E−76 | 1.4 | CX3CL1 | 5.97 | 2.20E−08 | 1.64 | LIPH | 3.83 | 6.88E−04 | 0.2 | PGM1 | 2.68 | 2.92E−02 | 0.13 |
| PLCB1 | 18.58 | 5.30E−75 | 1.14 | NOL3 | 5.97 | 2.23E−08 | 0.54 | TCEAL8 | 3.82 | 7.36E−04 | 1.16 | KCNQ3 | 2.67 | 2.99E−02 | 0.73 |
| MAML2 | 18.5 | 2.41E−74 | 0.62 | SLC25A6 | 5.96 | 2.29E−08 | 0.85 | LIPE | 3.82 | 7.15E−04 | 1.1 | POLR2G | 2.67 | 2.99E−02 | 0.68 |
| SLCO3A1 | 18.46 | 4.39E−74 | 0.98 | TEAD3 | 5.96 | 2.29E−08 | 0.56 | FAH | 3.82 | 7.29E−04 | 0.72 | METTL2A | 2.67 | 3.03E−02 | 0.35 |
| PKM | 18.32 | 5.62E−73 | 0.84 | TMBIM1 | 5.96 | 2.32E−08 | 0.39 | CLMP | 3.82 | 7.39E−04 | 0.58 | EPHB4 | 2.67 | 3.01E−02 | 0.23 |
| CALD1 | 18.25 | 2.03E−72 | 1.23 | C4orf3 | 5.95 | 2.43E−08 | 0.77 | ADAP1 | 3.82 | 7.17E−04 | 0.46 | CCT4 | 2.67 | 2.99E−02 | 0.19 |
| PARP9 | 18.18 | 7.52E−72 | 1.26 | IFT172 | 5.95 | 2.40E−08 | 0.69 | SART3 | 3.82 | 7.27E−04 | 0.34 | MAGT1 | 2.67 | 2.97E−02 | 0.18 |
| MYOF | 18 | 2.05E−70 | 0.6 | NEK11 | 5.95 | 2.41E−08 | 0.4 | LTBP3 | 3.82 | 7.25E−04 | 0.29 | PTGDS | 2.66 | 3.08E−02 | 1.86 |
| RPS17 | 17.98 | 2.60E−70 | 2.54 | RPS19BP1 | 5.94 | 2.64E−08 | 1.22 | ENTPD8 | 3.81 | 7.59E−04 | 1.51 | DNAJC18 | 2.66 | 3.10E−02 | 1.12 |
| PLAT | 17.81 | 5.45E−69 | 1.72 | ANAPC11 | 5.94 | 2.53E−08 | 1.07 | TRAPPC3 | 3.81 | 7.60E−04 | 0.53 | HS6ST3 | 2.66 | 3.09E−02 | 0.92 |
| ZNF544 | 17.73 | 2.40E−68 | 3.56 | ATP6AP1 | 5.94 | 2.64E−08 | 1.04 | OXLD1 | 3.81 | 7.58E−04 | 0.52 | TMEM86B | 2.66 | 3.06E−02 | 0.76 |
| BCL2L1 | 17.63 | 1.47E−67 | 0.7 | MT-ATP6 | 5.94 | 2.64E−08 | 0.41 | SPNS1 | 3.81 | 7.51E−04 | 0.48 | LRRC37A | 2.66 | 3.1E−02 | 0.69 |
| ARPC1A | 17.61 | 1.80E−67 | 3.17 | KCNT2 | 5.93 | 2.77E−08 | 1.45 | TIMP1 | 3.81 | 7.50E−04 | 0.38 | HTRA1 | 2.66 | 3.12E−02 | 0.58 |
| IQCH | 17.46 | 2.68E−66 | 1.51 | AQP5 | 5.93 | 2.79E−08 | 1.09 | PPM1G | 3.81 | 7.51E−04 | 0.36 | RIC8A | 2.66 | 3.13E−02 | 0.34 |
| RRBP1 | 17.42 | 5.46E−66 | 0.93 | NOP10 | 5.93 | 2.82E−08 | 1.07 | COL16A1 | 3.81 | 7.52E−04 | 0.27 | PTOV1 | 2.66 | 3.10E−02 | 0.32 |
| C9orf16 | 17.38 | 1.04E−65 | 2.17 | ODF3B | 5.93 | 2.77E−08 | 0.96 | LHB | 3.8 | 7.80E−04 | 1.69 | DGCR6 | 2.65 | 3.21E−02 | 7.4 |
| ZNF665 | 17.38 | 9.83E−66 | 1.85 | FIGN | 5.93 | 2.79E−08 | 0.91 | RNF217-AS1 | 3.8 | 7.86E−04 | 1.63 | HOTTIP | 2.65 | 3.21E−02 | 2.24 |
| HLA-C | 17.38 | 1.04E−65 | 1.36 | MFSD11 | 5.93 | 2.78E−08 | 0.44 | SERTAD2 | 3.8 | 7.82E−04 | 0.46 | DENND2A | 2.65 | 3.19E−02 | 0.76 |
| IGF2BP3 | 17.3 | 4.34E−65 | 0.88 | U2SURP | 5.93 | 2.79E−08 | 0.31 | RALYL | 3.8 | 7.73E−04 | 0.32 | SNRNP35 | 2.65 | 3.14E−02 | 0.35 |
| SERF2 | 17.29 | 5.25E−65 | 1.21 | RASAL1 | 5.92 | 2.98E−08 | 0.61 | KCTD20 | 3.8 | 7.86E−04 | 0.28 | RNF8 | 2.65 | 3.19E−02 | 0.26 |
| MYH11 | 17.11 | 1.08E−63 | 1.85 | TYMP | 5.92 | 2.84E−08 | 0.55 | KCNH1 | 3.79 | 8.26E−04 | 1.82 | CHMP4B | 2.65 | 3.21E−02 | 0.26 |
| CAMK1D | 17.11 | 9.85E−64 | 1.13 | RSRC2 | 5.92 | 2.92E−08 | 0.43 | MEA1 | 3.79 | 8.17E−04 | 1.48 | IPPK | 2.65 | 3.20E−02 | 0.24 |
| AC058791.1 | 17.03 | 4.18E−63 | 1.71 | C17orf99 | 5.91 | 3.11E−08 | 3.25 | NPIPB3 | 3.79 | 8.19E−04 | 1.19 | MFSD14B | 2.65 | 3.19E−02 | 0.21 |
| CTSH | 16.97 | 1.21E−62 | 1.8 | NPIPA1 | 5.91 | 3.03E−08 | 1.01 | CERCAM | 3.79 | 8.18E−04 | 1.05 | AGO2 | 2.65 | 3.14E−02 | 0.16 |
| SNHG19 | 16.83 | 1.23E−61 | 1.69 | COTL1 | 5.91 | 3.13E−08 | 0.62 | AGPAT1 | 3.79 | 8.06E−04 | 1.01 | MYOCD | 2.64 | 3.25E−02 | 1.56 |
| RPS27A | 16.78 | 2.88E−61 | 1.23 | SRP14 | 5.9 | 3.34E−08 | 0.75 | FBXL7 | 3.79 | 8.18E−04 | 0.64 | FBP2 | 2.64 | 3.28E−02 | 1.5 |
| NCOR2 | 16.65 | 2.38E−60 | 0.8 | MALL | 5.9 | 3.26E−08 | 0.66 | SLPI | 3.79 | 8.13E−04 | 0.59 | IFT22 | 2.64 | 3.24E−02 | 1.24 |
| ANXA1 | 16.6 | 5.62E−60 | 0.76 | CYB561 | 5.9 | 3.34E−08 | 0.61 | NTM | 3.79 | 8.22E−04 | 0.58 | GSTT2B | 2.64 | 3.26E−02 | 1.08 |
| RPL38 | 16.49 | 3.33E−59 | 1.77 | MYH9 | 5.9 | 3.34E−08 | 0.33 | CEP89 | 3.79 | 8.25E−04 | 0.36 | BRICD5 | 2.64 | 3.25E−02 | 0.81 |
| RANBP17 | 16.49 | 3.19E−59 | 0.78 | VEPH1 | 5.89 | 3.47E−08 | 1.26 | ZEB1 | 3.79 | 8.11E−04 | 0.31 | PLA2G15 | 2.64 | 3.25E−02 | 0.77 |
| GTF2IRD1 | 16.36 | 2.63E−58 | 0.85 | PRKAG1 | 5.89 | 3.45E−08 | 0.62 | SH3BP1 | 3.78 | 8.53E−04 | 4.06 | STK16 | 2.64 | 3.22E−02 | 0.65 |
| CA1 | 16.32 | 5.30E−58 | 1.94 | TNS1 | 5.88 | 3.63E−08 | 0.58 | ZNF688 | 3.78 | 8.53E−04 | 1.31 | SARM1 | 2.64 | 3.27E−02 | 0.4 |
| MT-CO1 | 16.12 | 1.31E−56 | 0.88 | RPL37 | 5.88 | 3.75E−08 | 0.58 | ORMDL2 | 3.78 | 8.45E−04 | 1.24 | RABL2B | 2.64 | 3.30E−02 | 0.4 |
| PPDPF | 16.08 | 2.64E−56 | 1.47 | SMARCE1 | 5.87 | 3.82E−08 | 3.06 | GRID2 | 3.78 | 8.36E−04 | 1.23 | SSR1 | 2.64 | 3.27E−02 | 0.26 |
| RPL27A | 16.07 | 2.66E−56 | 1.15 | RHBDF2 | 5.87 | 3.98E−08 | 0.47 | TMED7-TICAM2 | 3.78 | 8.38E−04 | 1.03 | CADM2 | 2.64 | 3.26E−02 | 0.24 |
| CRIP2 | 16.01 | 7.20E−56 | 1.22 | ARFGAP1 | 5.87 | 3.99E−08 | 0.41 | PILRA | 3.78 | 8.30E−04 | 0.73 | LINC01376 | 2.64 | 3.25E−02 | 0.21 |
| FKBP5 | 15.95 | 2.05E−55 | 0.69 | TIMM8B | 5.86 | 4.18E−08 | 1.41 | BCAS4 | 3.78 | 8.42E−04 | 0.56 | ERGIC1 | 2.64 | 3.23E−02 | 0.17 |
| DES | 15.94 | 2.28E−55 | 4.38 | SDC4 | 5.86 | 4.23E−08 | 0.49 | RETSAT | 3.78 | 8.55E−04 | 0.42 | KLHL5 | 2.64 | 3.29E−02 | 0.12 |
| AXL | 15.93 | 2.47E−55 | 1.7 | TPM1 | 5.86 | 4.24E−08 | 0.31 | NRBP1 | 3.78 | 8.42E−04 | 0.36 | CFI | 2.63 | 3.31E−02 | 1.73 |
| ARHGEF10 | 15.88 | 5.44E−55 | 2.7 | ATR | 5.86 | 4.12E−08 | 0.29 | OS9 | 3.78 | 8.42E−04 | 0.33 | MGAT4C | 2.63 | 3.34E−02 | 1.09 |
| MAPK10 | 15.77 | 3.24E−54 | 1.08 | NDUFA2 | 5.85 | 4.32E−08 | 1.28 | IQCE | 3.78 | 8.41E−04 | 0.28 | MUL1 | 2.63 | 3.31E−02 | 0.89 |
| BMPR1B | 15.74 | 4.78E−54 | 1.2 | SNRPC | 5.85 | 4.36E−08 | 1.16 | CIB1 | 3.78 | 8.33E−04 | 0.26 | STK19 | 2.63 | 3.33E−02 | 0.45 |
| PKHD1 | 15.73 | 6.00E−54 | 1.16 | BPTF | 5.85 | 4.38E−08 | 0.21 | CBWD3 | 3.77 | 8.67E−04 | 0.58 | BOK | 2.63 | 3.34E−02 | 0.41 |
| SMCR5 | 15.68 | 1.26E−53 | 1.78 | SSR3 | 5.84 | 4.50E−08 | 0.57 | ANK2 | 3.77 | 8.80E−04 | 0.45 | LINC00342 | 2.63 | 3.35E−02 | 0.4 |
| TMC4 | 15.66 | 1.70E−53 | 0.77 | CLDN16 | 5.83 | 5.00E−08 | 1.21 | PER3 | 3.77 | 8.71E−04 | 0.38 | ABCA12 | 2.63 | 3.34E−02 | 0.39 |
| RPS11 | 15.58 | 6.12E−53 | 1.1 | TGFBI | 5.83 | 4.95E−08 | 0.96 | BTG1 | 3.77 | 8.59E−04 | 0.32 | ACTRIA | 2.63 | 3.31E−02 | 0.35 |
| SPDYE16 | 15.56 | 8.03E−53 | 2.15 | ARPC5L | 5.83 | 4.94E−08 | 0.84 | MANBA | 3.77 | 8.69E−04 | 0.25 | NFKBIB | 2.63 | 3.33E−02 | 0.3 |
| MAP3K13 | 15.54 | 1.07E−52 | 0.6 | SDF4 | 5.83 | 5.04E−08 | 0.78 | ADSL | 3.76 | 8.98E−04 | 3.22 | SLC22A18 | 2.63 | 3.36E−02 | 0.25 |
| MET | 15.39 | 1.11E−51 | 0.59 | SBF2-AS1 | 5.83 | 4.83E−08 | 0.72 | GRIK1 | 3.76 | 9.15E−04 | 0.76 | RBBP8 | 2.63 | 3.35E−02 | 0.23 |
| BIRC3 | 15.38 | 1.37E−51 | 1.14 | PSMG3 | 5.83 | 5.00E−08 | 0.68 | FUNDC2 | 3.76 | 8.98E−04 | 0.68 | DRG1 | 2.63 | 3.35E−02 | 0.22 |
| ST3GAL3 | 15.33 | 2.78E−51 | 3.47 | CACNA1A | 5.82 | 5.15E−08 | 1.16 | APOLD1 | 3.76 | 9.03E−04 | 0.66 | SLC17A9 | 2.63 | 3.31E−02 | 0.17 |
| LCN2 | 15.33 | 2.71E−51 | 1.07 | TGFB1 | 5.82 | 5.08E−08 | 0.53 | WDR46 | 3.76 | 9.10E−04 | 0.64 | DMD | 2.63 | 3.39E−02 | −0.03 |
| UBC | 15.31 | 3.73E−51 | 0.6 | KPNA6 | 5.82 | 5.34E−08 | 0.43 | RAB9A | 3.76 | 8.95E−04 | 0.56 | SDHAF1 | 2.62 | 3.45E−02 | 1.33 |
| LINGO1 | 15.29 | 5.53E−51 | 0.76 | NEDD9 | 5.81 | 5.61E−08 | 0.39 | SIAH2 | 3.76 | 9.15E−04 | 0.45 | RNF26 | 2.62 | 3.40E−02 | 1.24 |
| ZNF90 | 15.17 | 3.38E−50 | 1.49 | SHC3 | 5.8 | 5.70E−08 | 1.06 | PIP4K2C | 3.76 | 9.17E−04 | 0.45 | DNAH9 | 2.62 | 3.45E−02 | 0.96 |
| LINC00511 | 15.17 | 3.33E−50 | 0.44 | CD109 | 5.79 | 6.26E−08 | 0.51 | PARK7 | 3.76 | 9.01E−04 | 0.39 | CWC15 | 2.62 | 3.45E−02 | 0.41 |
| KRT18 | 15.15 | 4.29E−50 | 0.86 | HOXB7 | 5.78 | 6.62E−08 | 1.66 | DNAH7 | 3.76 | 8.93E−04 | 0.34 | MUC4 | 2.62 | 3.48E−02 | 0.35 |
| POLR2L | 14.97 | 6.23E−49 | 1.81 | TMEM54 | 5.78 | 6.66E−08 | 1.29 | LMAN1 | 3.76 | 9.03E−04 | 0.34 | MTG2 | 2.62 | 3.45E−02 | 0.27 |
| RPS15 | 14.94 | 9.50E−49 | 1.13 | USP5 | 5.78 | 6.38E−08 | 0.89 | ARL4D | 3.75 | 9.29E−04 | 1.79 | CHCHD1 | 2.61 | 3.55E−02 | 0.57 |
| ZFHX3 | 14.93 | 1.13E−48 | 0.66 | CD99 | 5.78 | 6.64E−08 | 0.53 | TFPT | 3.75 | 9.38E−04 | 1.05 | TMEM9 | 2.61 | 3.56E−02 | 0.52 |
| RPL10 | 14.87 | 2.78E−48 | 0.7 | CD24 | 5.78 | 6.60E−08 | 0.48 | LRRC49 | 3.75 | 9.37E−04 | 0.6 | FAM32A | 2.61 | 3.56E−02 | 0.47 |
| MICAL2 | 14.77 | 1.18E−47 | 0.8 | ZNF500 | 5.77 | 7.08E−08 | 1.05 | YWHAH | 3.75 | 9.26E−04 | 0.56 | PKIA | 2.61 | 3.53E−02 | 0.38 |
| FLNA | 14.75 | 1.67E−47 | 1.02 | MRPL28 | 5.77 | 6.84E−08 | 0.97 | PCBP1 | 3.75 | 9.47E−04 | 0.4 | RPL22L1 | 2.61 | 3.52E−02 | 0.31 |
| RPL8 | 14.69 | 4.13E−47 | 1 | IRF7 | 5.77 | 6.96E−08 | 0.85 | RNPS1 | 3.75 | 9.57E−04 | 0.25 | UBE2J1 | 2.61 | 3.53E−02 | 0.26 |
| RPL37A | 14.64 | 8.74E−47 | 1.05 | SPARC | 5.77 | 6.88E−08 | 0.39 | SMPD1 | 3.74 | 9.89E−04 | 1.42 | MVB12A | 2.61 | 3.53E−02 | 0.25 |
| SPATS2L | 14.6 | 1.39E−46 | 0.48 | NPAS2 | 5.77 | 6.86E−08 | 0.18 | FBXL6 | 3.74 | 9.71E−04 | 0.66 | TSN | 2.61 | 3.49E−02 | 0.24 |
| LRRFIP1 | 14.6 | 1.57E−46 | 0.42 | AKAP6 | 5.76 | 7.13E−08 | 1.05 | LAMC1 | 3.74 | 9.62E−04 | 0.22 | ARL13B | 2.61 | 3.50E−02 | 0.13 |
| OR2A1-AS1 | 14.52 | 5.09E−46 | 3.8 | AGPAT2 | 5.76 | 7.38E−08 | 0.65 | DHRS12 | 3.73 | 1.00E−03 | 2.91 | GABBR2 | 2.6 | 3.57E−02 | 1.69 |
| TIMP3 | 14.51 | 5.12E−46 | 1.81 | PSMB5 | 5.75 | 7.74E−08 | 0.73 | TXNRD3 | 3.73 | 9.99E−04 | 0.73 | ZNF667-AS1 | 2.6 | 3.60E−02 | 1.6 |
| RPL19 | 14.48 | 9.01E−46 | 1.22 | ATF4 | 5.75 | 7.68E−08 | 0.45 | CIC | 3.73 | 1.02E−03 | 0.56 | LINC00908 | 2.6 | 3.60E−02 | 1.09 |
| HLA-A | 14.47 | 1.04E−45 | 0.85 | EIF1AX | 5.75 | 7.63E−08 | 0.43 | GALM | 3.73 | 1.01E−03 | 0.52 | GPM6B | 2.6 | 3.64E−02 | 0.64 |
| RPL31 | 14.46 | 1.08E−45 | 1.1 | WARS | 5.74 | 8.31E−08 | 0.71 | EXOC3 | 3.73 | 1.02E−03 | 0.46 | RNASEH2C | 2.6 | 3.64E−02 | 0.49 |
| RPS2 | 14.46 | 1.08E−45 | 0.99 | P2RX4 | 5.74 | 8.34E−08 | 0.66 | MAP2K7 | 3.73 | 1.00E−03 | 0.32 | TFIP11 | 2.6 | 3.64E−02 | 0.44 |
| ITGA3 | 14.41 | 2.42E−45 | 0.56 | PRRC2A | 5.74 | 8.10E−08 | 0.59 | RAB11B | 3.73 | 1.02E−03 | 0.31 | LINC01285 | 2.6 | 3.64E−02 | 0.43 |
| BCOR | 14.4 | 2.61E−45 | 1.44 | IL20RB | 5.74 | 8.10E−08 | 0.58 | ADH1C | 3.72 | 1.06E−03 | 4.23 | KEAP1 | 2.6 | 3.60E−02 | 0.36 |
| CD82 | 14.39 | 3.11E−45 | 1.31 | FANK1 | 5.74 | 8.35E−08 | 0.45 | PSMB2 | 3.72 | 1.04E−03 | 0.4 | CDK16 | 2.6 | 3.61E−02 | 0.31 |
| PARP14 | 14.39 | 2.82E−45 | 0.76 | COCH | 5.73 | 8.82E−08 | 1 | GNB2 | 3.72 | 1.04E−03 | 0.37 | NECAP2 | 2.6 | 3.62E−02 | 0.26 |
| MBOAT7 | 14.36 | 4.96E−45 | 1.39 | EVPL | 5.73 | 8.46E−08 | 0.63 | ELL | 3.72 | 1.05E−03 | 0.34 | POLDIP3 | 2.6 | 3.64E−02 | 0.26 |
| MT1E | 14.35 | 5.20E−45 | 2.55 | CYBA | 5.73 | 8.89E−08 | 0.47 | RNF149 | 3.72 | 1.04E−03 | 0.16 | ATP6V0B | 2.6 | 3.65E−02 | 0.24 |
| MX1 | 14.33 | 7.16E−45 | 1.54 | VEZT | 5.73 | 8.83E−08 | 0.24 | SPR | 3.71 | 1.10E−03 | 1.77 | WBP2NL | 2.6 | 3.66E−02 | 0.24 |
| RNASE10 | 14.29 | 1.18E−44 | 1.9 | LIMD2 | 5.72 | 9.11E−08 | 3.95 | XXYLT1 | 3.71 | 1.11E−03 | 0.4 | OSGIN2 | 2.6 | 3.61E−02 | 0.22 |
| SLC35A3 | 14.27 | 1.61E−44 | 3.19 | GADD45B | 5.72 | 9.06E−08 | 0.78 | HAT1 | 3.71 | 1.08E−03 | 0.35 | PNPLA2 | 2.6 | 3.60E−02 | 0.21 |
| SMIM22 | 14.27 | 1.60E−44 | 2.12 | TAZ | 5.72 | 9.08E−08 | 0.56 | SYMPK | 3.71 | 1.10E−03 | 0.28 | STX4 | 2.6 | 3.66E−02 | 0.2 |
| DGKG | 14.27 | 1.56E−44 | 1.67 | MAP2 | 5.72 | 9.12E−08 | 0.5 | MFN2 | 3.7 | 1.11E−03 | 0.36 | VPS37B | 2.6 | 3.66E−02 | 0.13 |
| FOXP2 | 14.26 | 1.78E−44 | 0.92 | MRPL51 | 5.71 | 9.51E−08 | 1.17 | FBRSL1 | 3.7 | 1.12E−03 | 0.35 | CRY1 | 2.6 | 3.59E−02 | 0.04 |
| PSAP | 14.19 | 4.81E−44 | 1.16 | NDUFB4 | 5.71 | 9.91E−08 | 0.77 | VIM | 3.7 | 1.14E−03 | 0.29 | LRTOMT | 2.59 | 3.71E−02 | 4.1 |
| MAPK8IP3 | 14.16 | 8.06E−44 | 0.75 | MT-CO2 | 5.71 | 9.78E−08 | 0.4 | SLC35B2 | 3.69 | 1.16E−03 | 1.27 | RAB37 | 2.59 | 3.76E−02 | 1.87 |
| FGD6 | 14.06 | 3.14E−43 | 0.62 | CLEC2D | 5.7 | 1.05E−07 | 0.57 | TSPYL1 | 3.69 | 1.18E−03 | 0.89 | GSG1L | 2.59 | 3.72E−02 | 1.59 |
| CFL1 | 14.02 | 5.20E−43 | 1.23 | CUTA | 5.69 | 1.07E−07 | 0.88 | NHP2 | 3.69 | 1.19E−03 | 0.86 | DNASE2 | 2.59 | 3.69E−02 | 1.06 |
| BCAS3 | 13.99 | 8.40E−43 | 0.49 | PPP4C | 5.69 | 1.09E−07 | 0.51 | NEK8 | 3.69 | 1.16E−03 | 0.67 | ATP8A2 | 2.59 | 3.72E−02 | 0.74 |
| PDXDC1 | 13.94 | 1.65E−42 | 0.5 | ZNF280D | 5.69 | 1.09E−07 | 0.42 | ZNF189 | 3.69 | 1.20E−03 | 0.51 | WTIP | 2.59 | 3.72E−02 | 0.57 |
| PPP2R2B | 13.92 | 2.06E−42 | 2.81 | TMEM184B | 5.69 | 1.12E−07 | 0.41 | DPM2 | 3.69 | 1.16E−03 | 0.48 | GJC1 | 2.59 | 3.71E−02 | 0.51 |
| ISG15 | 13.92 | 2.26E−42 | 2.27 | ARRDC1 | 5.69 | 1.12E−07 | 0.39 | H1FX | 3.69 | 1.17E−03 | 0.47 | ESYT1 | 2.59 | 3.66E−02 | 0.37 |
| SAMD4A | 13.84 | 6.66E−42 | 0.72 | ASAP3 | 5.68 | 1.12E−07 | 1.26 | SKP1 | 3.69 | 1.18E−03 | 0.32 | GANAB | 2.59 | 3.72E−02 | 0.2 |
| CRHR1 | 13.83 | 7.79E−42 | 2.97 | RAN | 5.67 | 1.24E−07 | 0.84 | MAP3K9 | 3.69 | 1.18E−03 | 0.3 | CORO2A | 2.59 | 3.69E−02 | 0.18 |
| CLDN1 | 13.82 | 9.27E−42 | 0.86 | ESF1 | 5.67 | 1.21E−07 | 0.4 | PGM2L1 | 3.69 | 1.16E−03 | 0.21 | TCEAL1 | 2.58 | 3.78E−02 | 1.37 |
| SPG7 | 13.8 | 1.22E−41 | 0.66 | CNN2 | 5.66 | 1.27E−07 | 0.59 | CPS1 | 3.68 | 1.23E−03 | 0.98 | HOTAIRM1 | 2.58 | 3.80E−02 | 0.99 |
| HSPB1 | 13.79 | 1.39E−41 | 0.81 | COL26A1 | 5.65 | 1.38E−07 | 1.92 | MAPKAPK5-AS1 | 3.68 | 1.24E−03 | 0.84 | SDC3 | 2.58 | 3.83E−02 | 0.79 |
| TMPRSS4 | 13.79 | 1.23E−41 | 0.67 | SLC39A4 | 5.65 | 1.36E−07 | 1.46 | SCN9A | 3.68 | 1.21E−03 | 0.72 | DHX37 | 2.58 | 3.78E−02 | 0.47 |
| MYH14 | 13.78 | 1.43E−41 | 0.58 | CRLF1 | 5.65 | 1.37E−07 | 1.34 | REXO2 | 3.68 | 1.20E−03 | 0.46 | TCEA3 | 2.58 | 3.82E−02 | 0.4 |
| RPL10A | 13.75 | 2.16E−41 | 1.03 | BHLHE41 | 5.65 | 1.36E−07 | 0.87 | PDIA5 | 3.68 | 1.24E−03 | 0.36 | ABTB1 | 2.58 | 3.78E−02 | 0.27 |
| COL4A2 | 13.73 | 3.05E−41 | 1.67 | RPL7 | 5.65 | 1.36E−07 | 0.47 | IGFBP3 | 3.68 | 1.20E−03 | 0.3 | ANKRD13A | 2.58 | 3.82E−02 | 0.27 |
| RPL35A | 13.72 | 3.49E−41 | 1.19 | LRRC37A2 | 5.64 | 1.43E−07 | 0.57 | CRLF3 | 3.68 | 1.21E−03 | 0.25 | PKD1 | 2.58 | 3.80E−02 | 0.25 |
| SPNS2 | 13.71 | 3.73E−41 | 1.66 | PLEKHH3 | 5.64 | 1.43E−07 | 0.55 | GATAD2A | 3.68 | 1.21E−03 | 0.22 | PRPF40B | 2.58 | 3.80E−02 | 0.24 |
| MLXIP | 13.66 | 8.16E−41 | 0.83 | UNC5B-AS1 | 5.63 | 1.51E−07 | 1.34 | PARP15 | 3.67 | 1.25E−03 | 2.31 | EIF4ENIF1 | 2.58 | 3.78E−02 | 0.21 |
| ATF6B | 13.62 | 1.29E−40 | 1.17 | MAGEF1 | 5.63 | 1.55E−07 | 1.34 | TNXB | 3.67 | 1.28E−03 | 1.4 | ODF2L | 2.58 | 3.83E−02 | 0.14 |
| CHD4 | 13.57 | 2.67E−40 | 1.65 | PSMC3 | 5.63 | 1.55E−07 | 1.22 | DESI1 | 3.67 | 1.25E−03 | 0.51 | FRMPD4 | 2.57 | 3.95E−02 | 0.98 |
| TIMP2 | 13.5 | 6.86E−40 | 0.92 | TMEM173 | 5.63 | 1.50E−07 | 0.95 | ARFIP2 | 3.67 | 1.27E−03 | 0.42 | OASL | 2.57 | 3.91E−02 | 0.94 |
| THSD4 | 13.5 | 6.53E−40 | 0.49 | COL8A1 | 5.62 | 1.63E−07 | 0.99 | GSTK1 | 3.67 | 1.27E−03 | 0.33 | ALKBH7 | 2.57 | 3.94E−02 | 0.77 |
| ANXA8L1 | 13.48 | 8.33E−40 | 2.94 | BCAT2 | 5.62 | 1.61E−07 | 0.86 | PPM1N | 3.66 | 1.32E−03 | 0.76 | PKD2L2 | 2.57 | 3.89E−02 | 0.71 |
| COL1A1 | 13.48 | 9.01E−40 | 0.66 | PGP | 5.62 | 1.57E−07 | 0.6 | ASRGL1 | 3.66 | 1.33E−03 | 0.56 | COQ7 | 2.57 | 3.90E−02 | 0.47 |
| RPS23 | 13.47 | 1.03E−39 | 1.18 | ATP7A | 5.61 | 1.75E−07 | 2.56 | SNRPB | 3.66 | 1.33E−03 | 0.54 | ZDHHC4 | 2.57 | 3.88E−02 | 0.42 |
| EPHB2 | 13.44 | 1.43E−39 | 1.33 | SAMD3 | 5.61 | 1.74E−07 | 2.17 | KARS | 3.66 | 1.30E−03 | 0.38 | UBFD1 | 2.57 | 3.91E−02 | 0.3 |
| PRSS8 | 13.43 | 1.59E−39 | 1.27 | UBE2L6 | 5.61 | 1.70E−07 | 1.35 | DUS1L | 3.66 | 1.30E−03 | 0.33 | SRA1 | 2.57 | 3.94E−02 | 0.23 |
| MGLL | 13.41 | 2.25E−39 | 0.64 | RPS3A | 5.61 | 1.74E−07 | 0.64 | DGKZ | 3.66 | 1.29E−03 | 0.31 | TRNAU1AP | 2.57 | 3.87E−02 | 0.19 |
| MID1 | 13.39 | 2.85E−39 | 0.7 | AK1 | 5.6 | 1.78E−07 | 1.86 | FOXO3 | 3.66 | 1.31E−03 | 0.16 | CHFR | 2.57 | 3.93E−02 | 0.12 |
| RPL34 | 13.38 | 3.07E−39 | 1.21 | PAK3 | 5.6 | 1.80E−07 | 1.68 | DCC | 3.65 | 1.38E−03 | 1.31 | GSN | 2.57 | 3.94E−02 | 0.12 |
| PIK3C2G | 13.38 | 3.30E−39 | 0.59 | ROR2 | 5.6 | 1.84E−07 | 0.73 | JAK3 | 3.65 | 1.34E−03 | 0.81 | GRM1 | 2.56 | 4.06E−02 | 2.44 |
| MYL6 | 13.31 | 7.94E−39 | 0.83 | TANGO2 | 5.6 | 1.83E−07 | 0.61 | TNPO2 | 3.65 | 1.36E−03 | 0.34 | MAPK7 | 2.56 | 4.04E−02 | 0.86 |
| MX2 | 13.26 | 1.63E−38 | 1.74 | RNF19B | 5.6 | 1.78E−07 | 0.57 | FBXL18 | 3.65 | 1.38E−03 | 0.34 | CLEC12A | 2.56 | 3.97E−02 | 0.81 |
| KRT17 | 13.23 | 2.36E−38 | 0.84 | PPP6R1 | 5.6 | 1.78E−07 | 0.56 | PRICKLE1 | 3.65 | 1.37E−03 | 0.29 | RAB32 | 2.56 | 4.03E−02 | 0.65 |
| RPS25 | 13.19 | 4.01E−38 | 1.23 | MS4A5 | 5.59 | 1.95E−07 | 2.21 | SET | 3.65 | 1.38E−03 | 0.29 | MRPS15 | 2.56 | 4.04E−02 | 0.56 |
| SEC62 | 13.17 | 5.03E−38 | 0.87 | PPIB | 5.59 | 1.86E−07 | 0.87 | ZDHHC14 | 3.65 | 1.37E−03 | 0.2 | SLCO1B1 | 2.56 | 3.98E−02 | 0.47 |
| PRSS23 | 13.13 | 8.37E−38 | 1.62 | C19orf66 | 5.59 | 1.88E−07 | 0.54 | RBM42 | 3.64 | 1.39E−03 | 0.93 | NIPSNAP1 | 2.56 | 3.99E−02 | 0.47 |
| DOK5 | 13.12 | 9.50E−38 | 2.39 | TUFM | 5.59 | 1.87E−07 | 0.46 | CCDC30 | 3.64 | 1.40E−03 | 0.54 | HYDIN | 2.56 | 4.02E−02 | 0.35 |
| COL1A2 | 13.1 | 1.28E−37 | 0.66 | CHMPIA | 5.58 | 2.06E−07 | 0.53 | AMT | 3.63 | 1.44E−03 | 1.86 | BAX | 2.56 | 4.04E−02 | 0.28 |
| RPS18 | 13.09 | 1.41E−37 | 1.3 | SNHG8 | 5.58 | 2.00E−07 | 0.5 | DSCAM | 3.63 | 1.47E−03 | 1.22 | MAFK | 2.56 | 4.02E−02 | 0.22 |
| TAGLN | 13.01 | 4.26E−37 | 1.48 | TSC2 | 5.58 | 2.00E−07 | 0.33 | MYL12B | 3.63 | 1.47E−03 | 0.35 | CEP112 | 2.56 | 4.04E−02 | 0.17 |
| SYNGR2 | 13.01 | 4.04E−37 | 1.22 | CAPS2 | 5.57 | 2.10E−07 | 0.64 | SURF4 | 3.63 | 1.48E−03 | 0.35 | AC100830.3 | 2.55 | 4.10E−02 | 2.91 |
| FN1 | 13.01 | 4.31E−37 | 0.76 | RTCB | 5.57 | 2.17E−07 | 0.54 | PTPN13 | 3.63 | 1.48E−03 | 0.2 | LAGE3 | 2.55 | 4.17E−02 | 1.33 |
| STAT1 | 13 | 4.52E−37 | 0.99 | SLC15A2 | 5.55 | 2.37E−07 | 1.28 | PYCARD | 3.62 | 1.53E−03 | 1.08 | CPXM2 | 2.55 | 4.17E−02 | 1.3 |
| RPS20 | 12.99 | 5.43E−37 | 0.82 | UBALD1 | 5.55 | 2.42E−07 | 0.99 | FAM210B | 3.62 | 1.50E−03 | 0.74 | MIEF1 | 2.55 | 4.11E−02 | 0.54 |
| MSLN | 12.96 | 7.42E−37 | 1.18 | RUNX2 | 5.55 | 2.39E−07 | 0.56 | ZNF577 | 3.62 | 1.54E−03 | 0.54 | SNAP29 | 2.55 | 4.12E−02 | 0.34 |
| TOMM7 | 12.94 | 9.63E−37 | 1.38 | FBXO28 | 5.55 | 2.39E−07 | 0.42 | UHRF1BP1L | 3.62 | 1.53E−03 | 0.35 | PA2G4 | 2.55 | 4.10E−02 | 0.33 |
| SERPINE1 | 12.94 | 1.03E−36 | 1.23 | SYNM | 5.54 | 2.46E−07 | 2.85 | SPEG | 3.61 | 1.60E−03 | 0.91 | ATP6V0D1 | 2.55 | 4.07E−02 | 0.32 |
| IFITM3 | 12.89 | 2.02E−36 | 1.39 | MIEN1 | 5.54 | 2.55E−07 | 1.18 | GATA3 | 3.61 | 1.58E−03 | 0.81 | RCC1 | 2.55 | 4.14E−02 | 0.2 |
| CORO1C | 12.86 | 2.75E−36 | 0.82 | C1orf122 | 5.54 | 2.45E−07 | 1.09 | GAS7 | 3.61 | 1.60E−03 | 0.61 | TMEM248 | 2.55 | 4.13E−02 | 0.19 |
| MMP3 | 12.84 | 3.52E−36 | 1.85 | GADD45GIP1 | 5.54 | 2.54E−07 | 1.02 | BID | 3.61 | 1.55E−03 | 0.42 | PRNCR1 | 2.54 | 4.18E−02 | 2.99 |
| SLC39A11 | 12.84 | 3.71E−36 | 0.57 | PDE4C | 5.53 | 2.69E−07 | 1.26 | SOD1 | 3.61 | 1.57E−03 | 0.41 | MRAS | 2.54 | 4.22E−02 | 1.6 |
| MYO3B | 12.83 | 4.17E−36 | 2.84 | CTNNA2 | 5.53 | 2.67E−07 | 1.17 | RALGDS | 3.61 | 1.60E−03 | 0.33 | NXT1 | 2.54 | 4.20E−02 | 1.05 |
| ELAVL1 | 12.82 | 4.84E−36 | 0.95 | PILRB | 5.53 | 2.70E−07 | 0.47 | SPINT1 | 3.61 | 1.59E−03 | 0.29 | CHODL | 2.54 | 4.21E−02 | 0.74 |
| CHPF | 12.76 | 1.00E−35 | 2.14 | RHBDD3 | 5.52 | 2.75E−07 | 1.05 | ATP1A1 | 3.61 | 1.58E−03 | 0.24 | CISD2 | 2.54 | 4.24E−02 | 0.42 |
| LINC01481 | 12.74 | 1.32E−35 | 3.14 | OLFM4 | 5.52 | 2.73E−07 | 0.92 | LINC01268 | 3.6 | 1.62E−03 | 1.85 | RRAGC | 2.54 | 4.26E−02 | 0.3 |
| TMBIM6 | 12.74 | 1.27E−35 | 0.92 | NUCB2 | 5.52 | 2.78E−07 | 0.82 | CCDC34 | 3.6 | 1.66E−03 | 0.99 | RNF103-CHMP3 | 2.54 | 4.23E−02 | 0.28 |
| TEX41 | 12.71 | 1.82E−35 | 2.47 | EI24 | 5.52 | 2.79E−07 | 0.71 | ZNF768 | 3.6 | 1.66E−03 | 0.82 | OTUD6B-AS1 | 2.54 | 4.19E−02 | 0.26 |
| MYL9 | 12.68 | 2.89E−35 | 1.65 | MAF1 | 5.51 | 2.95E−07 | 0.96 | KIF22 | 3.6 | 1.65E−03 | 0.58 | ARPC5 | 2.54 | 4.20E−02 | 0.26 |
| C16orf45 | 12.63 | 5.12E−35 | 0.74 | DRAM1 | 5.51 | 2.94E−07 | 0.81 | CIRL-AS1 | 3.6 | 1.62E−03 | 0.47 | SNRPD1 | 2.54 | 4.24E−02 | 0.26 |
| KCNQ5 | 12.6 | 7.12E−35 | 1.84 | AC026167.1 | 5.51 | 2.92E−07 | 0.46 | COL23A1 | 3.59 | 1.70E−03 | 1.59 | AGAP4 | 2.54 | 4.19E−02 | 0.19 |
| RHOC | 12.58 | 1.02E−34 | 1.29 | BCAR3 | 5.51 | 2.94E−07 | 0.33 | SPDYA | 3.59 | 1.67E−03 | 0.81 | INVS | 2.54 | 4.26E−02 | 0.16 |
| TRIM56 | 12.57 | 1.02E−34 | 0.74 | LRRC58 | 5.5 | 3.03E−07 | 0.56 | SLC6A16 | 3.59 | 1.69E−03 | 0.78 | GALNT6 | 2.54 | 4.27E−02 | 0.16 |
| COMMD7 | 12.54 | 1.62E−34 | 4.25 | RPL4 | 5.5 | 3.06E−07 | 0.45 | APMAP | 3.59 | 1.69E−03 | 0.58 | EFR3B | 2.53 | 4.34E−02 | 1.31 |
| SLC33A1 | 12.54 | 1.49E−34 | 3.57 | MT-ND3 | 5.5 | 3.04E−07 | 0.36 | GABARAPL1 | 3.59 | 1.72E−03 | 0.49 | ROGDI | 2.53 | 4.32E−02 | 0.62 |
| IGFBP7 | 12.52 | 1.91E−34 | 0.95 | ARHGAP42 | 5.5 | 3.03E−07 | 0.28 | DSE | 3.59 | 1.71E−03 | 0.43 | AFF3 | 2.53 | 4.28E−02 | 0.53 |
| OOEP | 12.5 | 2.69E−34 | 1.24 | HELZ2 | 5.49 | 3.32E−07 | 0.68 | NAT14 | 3.58 | 1.74E−03 | 1.05 | IKBKG | 2.53 | 4.38E−02 | 0.45 |
| HOXB3 | 12.5 | 2.68E−34 | 1.18 | DCAF7 | 5.49 | 3.25E−07 | 0.36 | STAG3 | 3.58 | 1.79E−03 | 0.96 | MPHOSPH10 | 2.53 | 4.36E−02 | 0.31 |
| IL1RAP | 12.48 | 3.33E−34 | 0.72 | MRPS7 | 5.48 | 3.51E−07 | 1.06 | APOL6 | 3.58 | 1.76E−03 | 0.51 | TIPARP | 2.53 | 4.33E−02 | 0.22 |
| AGRN | 12.44 | 5.08E−34 | 0.91 | TACSTD2 | 5.48 | 3.54E−07 | 0.57 | SERPINB5 | 3.58 | 1.73E−03 | 0.42 | TTBK2 | 2.53 | 4.38E−02 | 0.22 |
| TMC6 | 12.42 | 6.67E−34 | 0.98 | SEC22A | 5.48 | 3.44E−07 | 0.34 | ZNF264 | 3.58 | 1.75E−03 | 0.39 | SENP5 | 2.53 | 4.29E−02 | 0.15 |
| ARMC9 | 12.4 | 8.39E−34 | 0.86 | HOXB2 | 5.47 | 3.66E−07 | 2.37 | AKR1A1 | 3.58 | 1.73E−03 | 0.33 | CAP1 | 2.53 | 4.33E−02 | 0.15 |
| RABAC1 | 12.37 | 1.29E−33 | 1.65 | HYOU1 | 5.47 | 3.63E−07 | 1.36 | MBOAT1 | 3.58 | 1.78E−03 | 0.32 | POU5F1B | 2.52 | 4.40E−02 | 3.06 |
| GAPDH | 12.36 | 1.39E−33 | 0.82 | COPE | 5.47 | 3.68E−07 | 0.72 | C7orf50 | 3.58 | 1.75E−03 | 0.29 | BEND6 | 2.52 | 4.41E−02 | 2.19 |
| LITAF | 12.35 | 1.61E−33 | 0.88 | CCDC144NL-AS1 | 5.45 | 4.11E−07 | 1.22 | PKD2 | 3.58 | 1.76E−03 | 0.26 | LSP1 | 2.52 | 4.50E−02 | 1.9 |
| MT-CO3 | 12.31 | 2.60E−33 | 0.62 | HIRA | 5.45 | 4.02E−07 | 0.73 | PIM3 | 3.58 | 1.73E−03 | 0.24 | NOS1 | 2.52 | 4.47E−02 | 1.4 |
| RPS28 | 12.28 | 3.82E−33 | 1.48 | NAT9 | 5.45 | 4.09E−07 | 0.6 | MTDH | 3.58 | 1.73E−03 | 0.16 | G6PC3 | 2.52 | 4.43E−02 | 1.03 |
| FLOT1 | 12.28 | 3.91E−33 | 0.87 | MYDGF | 5.45 | 4.11E−07 | 0.54 | DGCR6L | 3.57 | 1.83E−03 | 1.39 | PPFIA3 | 2.52 | 4.40E−02 | 0.87 |
| AIG1 | 12.28 | 3.97E−33 | 0.71 | GCNT2 | 5.44 | 4.28E−07 | 0.49 | MRPL54 | 3.57 | 1.85E−03 | 1.24 | PDLIM3 | 2.52 | 4.50E−02 | 0.54 |
| PXN | 12.27 | 4.29E−33 | 0.82 | MAPKBP1 | 5.43 | 4.57E−07 | 0.52 | LARP6 | 3.57 | 1.84E−03 | 1.23 | ANKRD54 | 2.52 | 4.46E−02 | 0.52 |
| NME3 | 12.23 | 7.18E−33 | 0.83 | SAMD4B | 5.43 | 4.60E−07 | 0.38 | EIF2S2 | 3.57 | 1.83E−03 | 0.34 | DNAH17 | 2.52 | 4.40E−02 | 0.5 |
| GPC1 | 12.18 | 1.27E−32 | 1.35 | SLC35F1 | 5.42 | 4.75E−07 | 1.05 | GNPTAB | 3.57 | 1.84E−03 | 0.34 | PDCL | 2.52 | 4.45E−02 | 0.38 |
| TNFRSF12A | 12.14 | 2.08E−32 | 1.45 | ARHGAP26-AS1 | 5.42 | 4.88E−07 | 0.85 | PKP2 | 3.57 | 1.83E−03 | 0.23 | RPGRIP1L | 2.52 | 4.41E−02 | 0.37 |
| FAU | 12.1 | 3.26E−32 | 1.21 | IFT57 | 5.42 | 4.87E−07 | 0.65 | FAM3D | 3.56 | 1.91E−03 | 1.05 | RASSF4 | 2.52 | 4.46E−02 | 0.31 |
| EPB42 | 12.09 | 3.57E−32 | 1.92 | PLCE1 | 5.42 | 4.89E−07 | 0.46 | CISD3 | 3.56 | 1.86E−03 | 0.82 | MYADM | 2.52 | 4.50E−02 | 0.28 |
| DCBLD2 | 12.04 | 6.58E−32 | 0.64 | TAGLN2 | 5.42 | 4.86E−07 | 0.44 | ADCY2 | 3.56 | 1.87E−03 | 0.7 | IFT20 | 2.52 | 4.41E−02 | 0.22 |
| CHN2 | 12.01 | 1.04E−31 | 2.46 | COPB1 | 5.42 | 4.80E−07 | 0.36 | CDKL3 | 3.56 | 1.89E−03 | 0.51 | PCDH1 | 2.52 | 4.40E−02 | 0.17 |
| SNRPD2 | 12.01 | 9.79E−32 | 1.8 | ACTN1 | 5.42 | 4.91E−07 | 0.31 | WFDC3 | 3.56 | 1.92E−03 | 0.45 | LAMB3 | 2.52 | 4.50E−02 | 0.14 |
| UBA52 | 12 | 1.13E−31 | 1.18 | POLR2K | 5.41 | 4.95E−07 | 1.31 | H6PD | 3.56 | 1.86E−03 | 0.43 | FRMD6-AS2 | 2.51 | 4.51E−02 | 0.67 |
| IL10RB | 11.98 | 1.44E−31 | 2.62 | TMEM192 | 5.41 | 5.05E−07 | 0.83 | GMEB1 | 3.56 | 1.90E−03 | 0.41 | PHKG1 | 2.51 | 4.51E−02 | 0.58 |
| RPL12 | 11.98 | 1.46E−31 | 0.94 | MAX | 5.41 | 4.95E−07 | 0.33 | LGALS4 | 3.56 | 1.86E−03 | 0.3 | KCNJ14 | 2.51 | 4.51E−02 | 0.52 |
| RPL6 | 11.94 | 2.35E−31 | 1.01 | C16orf58 | 5.4 | 5.38E−07 | 0.68 | DISC1 | 3.56 | 1.92E−03 | 0.3 | FAM213B | 2.51 | 4.59E−02 | 0.37 |
| STAT2 | 11.94 | 2.32E−31 | 0.88 | APRT | 5.4 | 5.39E−07 | 0.64 | IL12RB2 | 3.55 | 1.93E−03 | 1.3 | NAA38 | 2.51 | 4.57E−02 | 0.25 |
| CALR | 11.93 | 2.60E−31 | 1.21 | SNU13 | 5.4 | 5.27E−07 | 0.55 | FAHD2A | 3.55 | 1.93E−03 | 0.59 | PITRM1 | 2.51 | 4.56E−02 | 0.23 |
| IFI44 | 11.92 | 2.98E−31 | 1.09 | C17orf67 | 5.39 | 5.67E−07 | 0.6 | ZNHIT1 | 3.55 | 1.97E−03 | 0.49 | S100PBP | 2.51 | 4.52E−02 | 0.21 |
| SLC7A5 | 11.86 | 6.03E−31 | 2.02 | PAAF1 | 5.39 | 5.55E−07 | 0.59 | H1F0 | 3.55 | 1.98E−03 | 0.39 | ASCC2 | 2.51 | 4.57E−02 | 0.18 |
| AP3S1 | 11.86 | 5.72E−31 | 0.74 | PHLPP2 | 5.39 | 5.63E−07 | 0.59 | TMEM123 | 3.55 | 1.93E−03 | 0.28 | SNRNP25 | 2.5 | 4.64E−02 | 1.19 |
| RBCK1 | 11.85 | 6.73E−31 | 0.89 | MAP3K5 | 5.39 | 5.50E−07 | 0.25 | CMTM4 | 3.55 | 1.93E−03 | 0.22 | CECR2 | 2.5 | 4.68E−02 | 0.95 |
| DEPDC5 | 11.76 | 1.80E−30 | 3.11 | KLF10 | 5.38 | 6.02E−07 | 0.71 | RGS17 | 3.54 | 2.01E−03 | 1.13 | POLR3H | 2.5 | 4.68E−02 | 0.71 |
| PLEC | 11.74 | 2.44E−30 | 0.43 | IFT140 | 5.38 | 6.05E−07 | 0.46 | SF3A2 | 3.54 | 2.04E−03 | 0.62 | NTNG1 | 2.5 | 4.61E−02 | 0.69 |
| FAM129A | 11.73 | 2.58E−30 | 1.39 | TMCC3 | 5.37 | 6.39E−07 | 0.54 | SHKBP1 | 3.54 | 1.99E−03 | 0.5 | USP11 | 2.5 | 4.65E−02 | 0.44 |
| RPS8 | 11.73 | 2.53E−30 | 0.9 | XAF1 | 5.37 | 6.34E−07 | 0.33 | MAP2K3 | 3.54 | 2.05E−03 | 0.33 | CALN1 | 2.5 | 4.66E−02 | 0.36 |
| RORA | 11.73 | 2.60E−30 | 0.53 | LIMK1 | 5.36 | 6.54E−07 | 0.8 | CFAP36 | 3.54 | 2.03E−03 | 0.32 | PEX6 | 2.5 | 4.65E−02 | 0.34 |
| BDNF-AS | 11.71 | 3.36E−30 | 1.11 | EHD4 | 5.36 | 6.61E−07 | 0.5 | KIAA1671 | 3.54 | 2.03E−03 | 0.2 | TMEM185A | 2.5 | 4.63E−02 | 0.33 |
| ZFP36L1 | 11.71 | 3.29E−30 | 0.5 | CASC15 | 5.36 | 6.45E−07 | 0.31 | PAQR7 | 3.53 | 2.09E−03 | 1 | DFFA | 2.5 | 4.64E−02 | 0.32 |
| AC072062.1 | 11.68 | 4.84E−30 | 2.47 | TF | 5.35 | 6.80E−07 | 1.37 | CCDC180 | 3.53 | 2.06E−03 | 0.94 | SRD5A1 | 2.5 | 4.72E−02 | 0.22 |
| TCF7L1 | 11.68 | 4.76E−30 | 1.61 | CREB5 | 5.35 | 6.88E−07 | 0.44 | ARL1 | 3.53 | 2.11E−03 | 0.65 | CLUH | 2.5 | 4.65E−02 | 0.19 |
| H2AFJ | 11.63 | 8.04E−30 | 0.92 | TPST1 | 5.35 | 6.91E−07 | 0.31 | SCARB1 | 3.53 | 2.09E−03 | 0.51 | ABI3BP | 2.5 | 4.66E−02 | 0.13 |
| IGFBP4 | 11.62 | 9.56E−30 | 1.1 | FAM49B | 5.35 | 6.80E−07 | 0.17 | UBTD1 | 3.53 | 2.07E−03 | 0.49 | SLC24A2 | 2.49 | 4.78E−02 | 1.37 |
| RARRES3 | 11.58 | 1.44E−29 | 2.19 | CAMK4 | 5.34 | 7.48E−07 | 1.32 | ARF6 | 3.53 | 2.06E−03 | 0.27 | GPC3 | 2.49 | 4.80E−02 | 0.98 |
| LGALS1 | 11.57 | 1.71E−29 | 1.51 | TMEM160 | 5.34 | 7.18E−07 | 0.94 | TPD52 | 3.53 | 2.09E−03 | 0.21 | PLTP | 2.49 | 4.85E−02 | 0.62 |
| AEBP1 | 11.54 | 2.49E−29 | 1.29 | MFGE8 | 5.34 | 7.40E−07 | 0.92 | CATSPERD | 3.52 | 2.16E−03 | 1.92 | GPANK1 | 2.49 | 4.85E−02 | 0.58 |
| LRRIQ1 | 11.53 | 2.68E−29 | 2.29 | ENY2 | 5.34 | 7.22E−07 | 0.52 | RBKS | 3.52 | 2.14E−03 | 0.71 | EID1 | 2.49 | 4.83E−02 | 0.26 |
| SH3TC1 | 11.53 | 2.57E−29 | 0.96 | SLC13A3 | 5.33 | 7.73E−07 | 0.92 | GRAMDIC | 3.52 | 2.16E−03 | 0.4 | TRIM47 | 2.49 | 4.77E−02 | 0.21 |
| RPL18 | 11.52 | 3.11E−29 | 0.99 | CA13 | 5.33 | 7.92E−07 | 0.58 | PRPF31 | 3.52 | 2.17E−03 | 0.34 | SLC16A5 | 2.49 | 4.79E−02 | 0.19 |
| CLN8 | 11.49 | 4.14E−29 | 3.12 | NMRAL1 | 5.33 | 7.86E−07 | 0.55 | CLASRP | 3.52 | 2.20E−03 | 0.33 | FRMD3 | 2.49 | 4.79E−02 | 0.19 |
| ZFAS1 | 11.44 | 7.26E−29 | 0.76 | MAP1LC3A | 5.33 | 7.72E−07 | 0.52 | C8orf59 | 3.52 | 2.19E−03 | 0.3 | PLEKHM1 | 2.49 | 4.77E−02 | 0.14 |
| ASS1 | 11.43 | 8.59E−29 | 1.34 | LPIN1 | 5.33 | 7.66E−07 | 0.4 | SOAT1 | 3.52 | 2.17E−03 | 0.28 | MT1M | 2.48 | 4.94E−02 | 1.64 |
| EEF1A1 | 11.41 | 1.08E−28 | 0.7 | ZYX | 5.32 | 8.05E−07 | 0.74 | CMSS1 | 3.52 | 2.15E−03 | 0.19 | LINC00942 | 2.48 | 4.93E−02 | 1.33 |
| RUVBL1 | 11.4 | 1.13E−28 | 0.97 | OTUB1 | 5.32 | 8.09E−07 | 0.59 | HSD17B14 | 3.51 | 2.28E−03 | 1.3 | CORO2B | 2.48 | 4.87E−02 | 1.29 |
| PCDH11X | 11.39 | 1.32E−28 | 1.92 | ARPC2 | 5.32 | 8.27E−07 | 0.25 | FLYWCH2 | 3.51 | 2.27E−03 | 0.99 | TMED1 | 2.48 | 4.86E−02 | 0.88 |
| DNASE1 | 11.39 | 1.35E−28 | 0.76 | MTCH1 | 5.32 | 8.10E−07 | 0.17 | ITGA9 | 3.51 | 2.24E−03 | 0.85 | CPLX1 | 2.48 | 4.94E−02 | 0.78 |
| EIF2B5 | 11.35 | 2.12E−28 | 1.05 | TMEM41A | 5.31 | 8.79E−07 | 0.48 | KAT8 | 3.51 | 2.27E−03 | 0.47 | C12orf43 | 2.48 | 4.96E−02 | 0.75 |
| KRT7 | 11.35 | 1.95E−28 | 0.52 | AP002884.2 | 5.3 | 9.11E−07 | 3.24 | ARSD | 3.51 | 2.23E−03 | 0.37 | RBP5 | 2.48 | 4.87E−02 | 0.72 |
| GFPT1 | 11.34 | 2.20E−28 | 0.59 | NOP2 | 5.3 | 8.95E−07 | 2.06 | SNX8 | 3.51 | 2.24E−03 | 0.34 | ZNF524 | 2.48 | 4.91E−02 | 0.69 |
| SPSB3 | 11.28 | 4.43E−28 | 0.86 | EMP1 | 5.3 | 9.28E−07 | 0.41 | ITFG2 | 3.51 | 2.26E−03 | 0.25 | PPP2R5B | 2.48 | 4.96E−02 | 0.35 |
| C4orf36 | 11.26 | 5.81E−28 | 2.64 | HPS4 | 5.3 | 8.92E−07 | 0.38 | IGF2BP2 | 3.51 | 2.27E−03 | 0.01 | TRAM2 | 2.48 | 4.90E−02 | 0.33 |
| ZNFX1 | 11.26 | 5.80E−28 | 0.78 | TAP1 | 5.29 | 9.57E−07 | 0.67 | BOLA3 | 3.5 | 2.34E−03 | 1.55 | FBRS | 2.48 | 4.86E−02 | 0.3 |
| DDR1 | 11.19 | 1.28E−27 | 1.01 | DPP10 | 5.29 | 9.38E−07 | 0.64 | DIXDC1 | 3.5 | 2.31E−03 | 0.54 | RAB8B | 2.48 | 4.95E−02 | 0.15 |
| NAPA | 11.18 | 1.43E−27 | 1.11 | FRMD8 | 5.29 | 9.43E−07 | 0.49 | COX7A2 | 3.5 | 2.34E−03 | 0.48 | SMIM24 | 2.47 | 5.08E−02 | 1.42 |
| BCAP31 | 11.16 | 1.77E−27 | 1.1 | PCDH15 | 5.28 | 1.01E−06 | 1.04 | ABCA13 | 3.5 | 2.35E−03 | 0.25 | CFAP57 | 2.47 | 5.00E−02 | 1.35 |
| XPO5 | 11.16 | 1.78E−27 | 0.83 | PSMC5 | 5.28 | 1.03E−06 | 1.01 | CLCN7 | 3.5 | 2.34E−03 | 0.24 | TOX2 | 2.47 | 4.99E−02 | 0.84 |
| COL6A2 | 11.15 | 1.95E−27 | 1.17 | PRELID1 | 5.28 | 1.02E−06 | 0.76 | TMEM254-AS1 | 3.49 | 2.37E−03 | 1.7 | CITED2 | 2.47 | 5.07E−02 | 0.77 |
| RPS9 | 11.15 | 1.87E−27 | 0.68 | PTDSS1 | 5.28 | 1.01E−06 | 0.73 | MCOLN3 | 3.49 | 2.38E−03 | 1.57 | SERPING1 | 2.47 | 5.00E−02 | 0.72 |
| TUBB | 11.13 | 2.39E−27 | 1.29 | MED10 | 5.28 | 9.88E−07 | 0.51 | LRMP | 3.49 | 2.38E−03 | 0.52 | MTTP | 2.47 | 5.00E−02 | 0.57 |
| TNFSF10 | 11.13 | 2.31E−27 | 1.1 | PRSS21 | 5.27 | 1.08E−06 | 1.26 | VPS4A | 3.49 | 2.41E−03 | 0.46 | TMUB1 | 2.47 | 5.07E−02 | 0.36 |
| BAIAP2L2 | 11.13 | 2.25E−27 | 0.78 | IFIH1 | 5.27 | 1.06E−06 | 0.74 | ARCN1 | 3.49 | 2.39E−03 | 0.34 | FCHO1 | 2.47 | 5.09E−02 | 0.36 |
| SIK3 | 11.12 | 2.57E−27 | 0.4 | COX6C | 5.27 | 1.05E−06 | 0.67 | SLC25A39 | 3.49 | 2.43E−03 | 0.32 | APH1A | 2.47 | 5.08E−02 | 0.3 |
| LGALS3BP | 11.09 | 3.58E−27 | 1.22 | ISPD | 5.27 | 1.07E−06 | 0.4 | ANKRD36B | 3.49 | 2.40E−03 | 0.24 | MFSD12 | 2.47 | 5.05E−02 | 0.22 |
| KCNK5 | 11.05 | 5.93E−27 | 1.33 | MED15 | 5.27 | 1.06E−06 | 0.31 | ASAP1 | 3.49 | 2.42E−03 | 0.14 | LAD1 | 2.47 | 5.08E−02 | 0.22 |
| MAMDC2 | 11.04 | 6.37E−27 | 1.08 | CCDC167 | 5.25 | 1.17E−06 | 1.63 | C21orf2 | 3.48 | 2.50E−03 | 0.5 | SLC25A3 | 2.47 | 5.09E−02 | 0.22 |
| FIRRE | 11.04 | 6.27E−27 | 1.02 | NENF | 5.25 | 1.20E−06 | 1.07 | BCL7A | 3.48 | 2.50E−03 | 0.41 | SLC2A4RG | 2.47 | 4.98E−02 | 0.17 |
| AMPD3 | 11.02 | 7.51E−27 | 1.42 | DDX54 | 5.25 | 1.20E−06 | 0.98 | PHF1 | 3.48 | 2.50E−03 | 0.41 | PISD | 2.47 | 5.09E−02 | 0.14 |
| SPDYE5 | 11 | 1.03E−26 | 1.31 | FBXO36 | 5.25 | 1.20E−06 | 0.66 | ATP10D | 3.48 | 2.48E−03 | 0.34 | TP53RK | 2.46 | 5.15E−02 | 1.13 |
| CTSZ | 10.98 | 1.25E−26 | 1.22 | TSPAN1 | 5.25 | 1.17E−06 | 0.36 | S100A11 | 3.48 | 2.47E−03 | 0.25 | HMCN2 | 2.46 | 5.20E−02 | 0.79 |
| RPN1 | 10.96 | 1.52E−26 | 1.19 | PIR | 5.24 | 1.22E−06 | 0.82 | SEC14L1 | 3.48 | 2.51E−03 | 0.24 | EIF2B3 | 2.46 | 5.11E−02 | 0.28 |
| ATXN2 | 10.96 | 1.60E−26 | 0.44 | IRAK1 | 5.24 | 1.22E−06 | 0.58 | RNMT | 3.48 | 2.47E−03 | 0.21 | CREB3 | 2.46 | 5.11E−02 | 0.24 |
| CYP7B1 | 10.95 | 1.66E−26 | 1.15 | PCYTIA | 5.24 | 1.23E−06 | 0.56 | MAP7D1 | 3.48 | 2.53E−03 | 0.2 | DGUOK | 2.46 | 5.17E−02 | 0.23 |
| RPS14 | 10.95 | 1.75E−26 | 0.92 | CBLC | 5.24 | 1.22E−06 | 0.45 | TRAP1 | 3.48 | 2.46E−03 | 0.17 | ITPR2 | 2.46 | 5.12E−02 | 0.1 |
| PFDN5 | 10.95 | 1.64E−26 | 0.89 | UQCRHL | 5.23 | 1.31E−06 | 2.03 | PCBD1 | 3.47 | 2.57E−03 | 0.84 | TRIO | 2.46 | 5.22E−02 | 0.02 |
| TAPBP | 10.94 | 1.85E−26 | 0.75 | MMP25-AS1 | 5.23 | 1.31E−06 | 0.95 | AMOTL2 | 3.47 | 2.55E−03 | 0.78 | AC159540.2 | 2.45 | 5.24E−02 | 3.16 |
| MIR99AHG | 10.93 | 2.15E−26 | 1.29 | PHLDA3 | 5.23 | 1.30E−06 | 0.73 | TMEM9B | 3.47 | 2.58E−03 | 0.71 | CGB7 | 2.45 | 5.30E−02 | 2.15 |
| SSR4 | 10.9 | 2.88E−26 | 1.64 | ZNF512B | 5.23 | 1.32E−06 | 0.7 | IDH3B | 3.47 | 2.56E−03 | 0.67 | WDR63 | 2.45 | 5.25E−02 | 1.27 |
| RPS6KA2 | 10.9 | 2.88E−26 | 0.71 | PSMC4 | 5.23 | 1.28E−06 | 0.69 | CFAP70 | 3.47 | 2.57E−03 | 0.55 | TSPAN2 | 2.45 | 5.25E−02 | 0.87 |
| RCN1 | 10.87 | 4.10E−26 | 1.78 | ATP2B1 | 5.23 | 1.30E−06 | 0.43 | C2orf68 | 3.47 | 2.56E−03 | 0.43 | FIBP | 2.45 | 5.37E−02 | 0.77 |
| RPS12 | 10.85 | 4.86E−26 | 1.01 | MIR646HG | 5.22 | 1.37E−06 | 1.37 | EIF3D | 3.47 | 2.58E−03 | 0.32 | TXK | 2.45 | 5.26E−02 | 0.67 |
| KDELR1 | 10.84 | 5.57E−26 | 1.18 | MARS | 5.22 | 1.34E−06 | 0.64 | CLTA | 3.47 | 2.61E−03 | 0.24 | ZNF519 | 2.45 | 5.32E−02 | 0.41 |
| OARD1 | 10.82 | 6.53E−26 | 1.24 | AP1B1 | 5.22 | 1.34E−06 | 0.6 | MCCC1 | 3.47 | 2.59E−03 | 0.23 | CYCS | 2.45 | 5.27E−02 | 0.36 |
| SPATS2 | 10.8 | 8.31E−26 | 0.63 | CHST11 | 5.22 | 1.34E−06 | 0.33 | UBA7 | 3.46 | 2.71E−03 | 0.67 | GTF2A2 | 2.45 | 5.24E−02 | 0.29 |
| MEG3 | 10.79 | 9.41E−26 | 3.33 | IGF2 | 5.21 | 1.43E−06 | 7.79 | SLC7A6OS | 3.46 | 2.65E−03 | 0.46 | OPA3 | 2.45 | 5.35E−02 | 0.21 |
| CHKA | 10.79 | 9.53E−26 | 0.58 | RHBDD2 | 5.21 | 1.45E−06 | 1 | TMEM106B | 3.46 | 2.64E−03 | 0.38 | XBP1 | 2.45 | 5.26E−02 | 0.13 |
| CNKSR3 | 10.78 | 1.01E−25 | 0.84 | LOXL1 | 5.21 | 1.43E−06 | 0.98 | HERPUD1 | 3.46 | 2.67E−03 | 0.32 | C16orf96 | 2.44 | 5.49E−02 | 2.14 |
| FTL | 10.77 | 1.21E−25 | 0.72 | CHMP3 | 5.21 | 1.43E−06 | 0.91 | KIAA1841 | 3.46 | 2.69E−03 | 0.29 | TMEM99 | 2.44 | 5.45E−02 | 0.72 |
| SLC26A3 | 10.77 | 1.21E−25 | 0.65 | MROH6 | 5.21 | 1.45E−06 | 0.42 | WNT10A | 3.45 | 2.76E−03 | 1.16 | PHYHD1 | 2.44 | 5.47E−02 | 0.49 |
| COL3A1 | 10.77 | 1.21E−25 | 0.6 | CLN5 | 5.2 | 1.51E−06 | 2.75 | PPIL6 | 3.45 | 2.74E−03 | 0.7 | C20orf96 | 2.44 | 5.46E−02 | 0.48 |
| TXNRD1 | 10.75 | 1.39E−25 | 0.67 | FGF1 | 5.2 | 1.54E−06 | 1.46 | TXN2 | 3.45 | 2.78E−03 | 0.58 | PQLC1 | 2.44 | 5.46E−02 | 0.32 |
| SVIL | 10.72 | 1.92E−25 | 0.45 | STX5 | 5.2 | 1.55E−06 | 0.39 | LINC00276 | 3.45 | 2.79E−03 | 0.57 | IRF1 | 2.44 | 5.49E−02 | 0.32 |
| ZNF254 | 10.68 | 3.05E−25 | 0.74 | ZNF593 | 5.19 | 1.64E−06 | 1.22 | RRP12 | 3.45 | 2.78E−03 | 0.26 | TNFAIP1 | 2.44 | 5.39E−02 | 0.26 |
| PTPRS | 10.66 | 3.89E−25 | 1.26 | ZNF260 | 5.18 | 1.68E−06 | 0.71 | MBD4 | 3.45 | 2.72E−03 | 0.23 | ARHGDIA | 2.44 | 5.47E−02 | 0.24 |
| RPL27 | 10.66 | 3.65E−25 | 1.13 | ANTXR1 | 5.18 | 1.68E−06 | 0.69 | MAP3K19 | 3.44 | 2.86E−03 | 1.17 | TRADD | 2.44 | 5.43E−02 | 0.22 |
| CDH4 | 10.63 | 4.97E−25 | 2.21 | TAF11 | 5.18 | 1.67E−06 | 0.49 | TMEM217 | 3.44 | 2.86E−03 | 1.01 | HIST3H2A | 2.43 | 5.55E−02 | 0.85 |
| THBS1 | 10.61 | 6.11E−25 | 1.24 | SLC16A3 | 5.18 | 1.71E−06 | 0.34 | COX6B2 | 3.44 | 2.88E−03 | 0.74 | MORN4 | 2.43 | 5.59E−02 | 0.61 |
| ONECUT2 | 10.6 | 7.04E−25 | 0.77 | HDAC7 | 5.17 | 1.74E−06 | 0.29 | MUC12 | 3.44 | 2.88E−03 | 0.72 | PPP5D1 | 2.43 | 5.52E−02 | 0.57 |
| ACTG2 | 10.59 | 8.10E−25 | 3.76 | PPP1CA | 5.16 | 1.86E−06 | 0.7 | HEBP1 | 3.44 | 2.87E−03 | 0.66 | EBNA1BP2 | 2.43 | 5.55E−02 | 0.43 |
| KIZ | 10.58 | 8.44E−25 | 1.01 | CXXC5 | 5.16 | 1.91E−06 | 0.53 | MAZ | 3.44 | 2.86E−03 | 0.58 | RTN2 | 2.43 | 5.55E−02 | 0.41 |
| TMCC1 | 10.58 | 8.24E−25 | 0.35 | TAOK3 | 5.16 | 1.88E−06 | 0.21 | ARSA | 3.44 | 2.85E−03 | 0.54 | LAMTOR1 | 2.43 | 5.56E−02 | 0.37 |
| SNHG9 | 10.57 | 9.21E−25 | 1.61 | SNRPB2 | 5.15 | 1.98E−06 | 0.84 | PEX11B | 3.44 | 2.84E−03 | 0.39 | ZNF598 | 2.43 | 5.63E−02 | 0.32 |
| RPS15A | 10.56 | 1.12E−24 | 1.46 | PRKD3 | 5.15 | 1.95E−06 | 0.48 | AGR3 | 3.44 | 2.84E−03 | 0.3 | PLEKHG6 | 2.43 | 5.55E−02 | 0.3 |
| CLSTN3 | 10.56 | 1.09E−24 | 1.06 | ZNF331 | 5.14 | 2.05E−06 | 0.81 | CELSR1 | 3.44 | 2.86E−03 | 0.26 | RARA | 2.43 | 5.51E−02 | 0.27 |
| NRP2 | 10.56 | 1.04E−24 | 0.89 | PLAA | 5.14 | 2.12E−06 | 0.58 | TAF1D | 3.44 | 2.88E−03 | 0.17 | MED21 | 2.43 | 5.60E−02 | 0.27 |
| PDE4D | 10.55 | 1.16E−24 | 0.38 | SF3B2 | 5.14 | 2.08E−06 | 0.46 | RARRES2 | 3.43 | 2.99E−03 | 1.34 | C6orf62 | 2.43 | 5.52E−02 | 0.18 |
| CLDN4 | 10.52 | 1.60E−24 | 0.69 | NCMAP | 5.13 | 2.17E−06 | 0.67 | RNF157 | 3.43 | 2.96E−03 | 0.56 | ACAP3 | 2.43 | 5.54E−02 | 0.17 |
| MT-ND1 | 10.5 | 1.94E−24 | 0.82 | SLC47A1 | 5.12 | 2.27E−06 | 1.26 | USF3 | 3.43 | 2.94E−03 | 0.36 | RGS12 | 2.43 | 5.52E−02 | 0.13 |
| CCDC40 | 10.48 | 2.55E−24 | 1.44 | SLC52A2 | 5.12 | 2.32E−06 | 0.85 | UBTF | 3.43 | 2.94E−03 | 0.32 | LINC01494 | 2.42 | 5.71E−02 | 1.95 |
| TP63 | 10.48 | 2.38E−24 | 1 | TOMM40 | 5.12 | 2.32E−06 | 0.79 | GLI2 | 3.43 | 2.97E−03 | 0.28 | ZAN | 2.42 | 5.69E−02 | 1.91 |
| CAST | 10.48 | 2.49E−24 | 0.43 | FAM114A1 | 5.12 | 2.28E−06 | 0.34 | C6orf47 | 3.42 | 3.07E−03 | 1.3 | MAPK8IP1 | 2.42 | 5.69E−02 | 1.13 |
| ZNF320 | 10.44 | 3.73E−24 | 3.48 | KLF12 | 5.12 | 2.24E−06 | 0.29 | ORAI3 | 3.42 | 3.07E−03 | 1.06 | DNPH1 | 2.42 | 5.72E−02 | 0.8 |
| RPL23 | 10.42 | 4.57E−24 | 0.94 | TBCB | 5.11 | 2.43E−06 | 0.58 | WDR54 | 3.42 | 3.07E−03 | 1.05 | ACD | 2.42 | 5.66E−02 | 0.76 |
| SLC35F3 | 10.42 | 4.81E−24 | 0.72 | PRKAR1B | 5.11 | 2.44E−06 | 0.55 | CNTNAP5 | 3.42 | 3.10E−03 | 0.95 | PPIL3 | 2.42 | 5.64E−02 | 0.37 |
| DTD1 | 10.41 | 5.12E−24 | 2.28 | APOL1 | 5.11 | 2.38E−06 | 0.35 | TXNRD2 | 3.42 | 3.08E−03 | 0.5 | KHDC1 | 2.42 | 5.70E−02 | 0.31 |
| SYTL3 | 10.41 | 4.83E−24 | 1.15 | ITGA5 | 5.1 | 2.49E−06 | 0.95 | SPATA33 | 3.42 | 3.10E−03 | 0.48 | SMARCD2 | 2.42 | 5.71E−02 | 0.23 |
| RPL23A | 10.39 | 6.31E−24 | 1.05 | ZNF302 | 5.1 | 2.59E−06 | 0.41 | KANK2 | 3.42 | 3.07E−03 | 0.45 | GSS | 2.42 | 5.70E−02 | 0.22 |
| COPG1 | 10.39 | 6.07E−24 | 0.98 | RAB40C | 5.1 | 2.50E−06 | 0.35 | ARL8A | 3.42 | 3.07E−03 | 0.44 | EAPP | 2.42 | 5.68E−02 | 0.18 |
| NOL4L | 10.38 | 6.81E−24 | 0.89 | SLC30A8 | 5.09 | 2.65E−06 | 2.1 | AACS | 3.42 | 3.03E−03 | 0.39 | ATP6V0E1 | 2.42 | 5.74E−02 | 0.18 |
| PDLIM7 | 10.37 | 7.36E−24 | 0.54 | SLC26A2 | 5.09 | 2.69E−06 | 1.07 | VAC14 | 3.42 | 3.02E−03 | 0.3 | CMAS | 2.42 | 5.63E−02 | 0.16 |
| NAPRT | 10.36 | 8.80E−24 | 0.83 | DMPK | 5.09 | 2.70E−06 | 0.61 | KIF9-AS1 | 3.42 | 3.08E−03 | 0.27 | PLEKHM2 | 2.42 | 5.72E−02 | 0.15 |
| NBEA | 10.36 | 8.93E−24 | 0.62 | PPM1K | 5.08 | 2.83E−06 | 0.99 | STAG1 | 3.42 | 3.08E−03 | 0.1 | TSPEAR | 2.41 | 5.85E−02 | 1.9 |
| COX6B1 | 10.33 | 1.14E−23 | 1.36 | PTK7 | 5.08 | 2.77E−06 | 0.54 | TMEM11 | 3.41 | 3.16E−03 | 1 | UNC13C | 2.41 | 5.83E−02 | 1.56 |
| RPS13 | 10.32 | 1.32E−23 | 1.15 | PDZRN4 | 5.07 | 2.89E−06 | 2.59 | EML5 | 3.41 | 3.12E−03 | 0.94 | LSM10 | 2.41 | 5.90E−02 | 1.17 |
| ARPC1B | 10.32 | 1.23E−23 | 1 | SPATA20 | 5.07 | 2.95E−06 | 0.53 | SIVA1 | 3.41 | 3.21E−03 | 0.69 | HPSE2 | 2.41 | 5.90E−02 | 1 |
| LAMA5 | 10.31 | 1.42E−23 | 0.6 | TP53I11 | 5.06 | 3.10E−06 | 1.11 | IRS1 | 3.41 | 3.13E−03 | 0.26 | COL4A3 | 2.41 | 5.91E−02 | 0.69 |
| URM1 | 10.3 | 1.57E−23 | 0.98 | EIF5A | 5.06 | 3.05E−06 | 0.89 | ITGA11 | 3.4 | 3.32E−03 | 1.37 | HPS3 | 2.41 | 5.79E−02 | 0.22 |
| COX7C | 10.29 | 1.75E−23 | 1.27 | HLAIDQB1 | 5.05 | 3.28E−06 | 1.77 | CETN2 | 3.4 | 3.30E−03 | 1.28 | FERMT2 | 2.41 | 5.82E−02 | 0.12 |
| C11orf54 | 10.27 | 2.10E−23 | 3.61 | DZIP1 | 5.05 | 3.19E−06 | 1.58 | RFX8 | 3.4 | 3.23E−03 | 1.23 | APOBEC3G | 2.4 | 6.01E−02 | 1.27 |
| ARL17A | 10.27 | 2.03E−23 | 1.02 | DDAH2 | 5.05 | 3.23E−06 | 0.79 | GYG1 | 3.4 | 3.21E−03 | 0.81 | ICMT | 2.4 | 5.94E−02 | 0.6 |
| OST4 | 10.24 | 2.85E−23 | 1.69 | SLC9A3R1 | 5.05 | 3.31E−06 | 0.78 | ZDHHC1 | 3.4 | 3.32E−03 | 0.61 | MED22 | 2.4 | 6.01E−02 | 0.59 |
| P3H4 | 10.2 | 4.17E−23 | 1.6 | TLDC1 | 5.05 | 3.20E−06 | 0.5 | HTATIP2 | 3.4 | 3.22E−03 | 0.45 | TPGS1 | 2.4 | 6.00E−02 | 0.52 |
| MRPS34 | 10.19 | 4.76E−23 | 1.4 | ZNF608 | 5.05 | 3.23E−06 | 0.31 | XPNPEP3 | 3.4 | 3.30E−03 | 0.3 | PREB | 2.4 | 6.04E−02 | 0.49 |
| MYEOV | 10.18 | 5.17E−23 | 0.48 | CACNB1 | 5.04 | 3.35E−06 | 0.97 | ITGB5-AS1 | 3.4 | 3.23E−03 | 0.19 | ZNF639 | 2.4 | 5.99E−02 | 0.41 |
| FXN | 10.17 | 5.68E−23 | 3.63 | BSDC1 | 5.04 | 3.38E−06 | 0.49 | BPGM | 3.39 | 3.37E−03 | 1.1 | DOK4 | 2.4 | 5.97E−02 | 0.34 |
| ARHGAP11B | 10.17 | 6.10E−23 | 3.19 | NUP107 | 5.04 | 3.45E−06 | 0.32 | CNPY3 | 3.39 | 3.36E−03 | 0.71 | PKIG | 2.4 | 6.04E−02 | 0.27 |
| NBL1 | 10.16 | 6.25E−23 | 1.2 | IQSEC2 | 5.03 | 3.66E−06 | 0.68 | EMP3 | 3.39 | 3.40E−03 | 0.71 | DAD1 | 2.4 | 6.01E−02 | 0.22 |
| TPCN1 | 10.16 | 6.16E−23 | 0.68 | MRPS12 | 5.02 | 3.84E−06 | 1.52 | ZNF16 | 3.39 | 3.39E−03 | 0.58 | SCAMP2 | 2.4 | 5.97E−02 | 0.18 |
| RAB31 | 10.11 | 1.05E−22 | 1.01 | CD40 | 5.02 | 3.85E−06 | 1.28 | EVI5L | 3.39 | 3.34E−03 | 0.45 | PPP2R3A | 2.4 | 5.95E−02 | 0.12 |
| FKBP2 | 10.1 | 1.12E−22 | 1.23 | CDA | 5.02 | 3.72E−06 | 0.88 | RBM8A | 3.39 | 3.34E−03 | 0.41 | LAT2 | 2.39 | 6.10E−02 | 0.8 |
| CA3 | 10.1 | 1.13E−22 | 0.99 | FAM96B | 5.02 | 3.75E−06 | 0.87 | NUDT4 | 3.39 | 3.34E−03 | 0.29 | TMEM101 | 2.39 | 6.09E−02 | 0.72 |
| GPR75-ASB3 | 10.09 | 1.28E−22 | 1.12 | ARL4C | 5.02 | 3.84E−06 | 0.69 | GPX3 | 3.38 | 3.45E−03 | 2.07 | SLC35A4 | 2.39 | 6.17E−02 | 0.61 |
| ROBO2 | 10.08 | 1.42E−22 | 1.25 | BCL7C | 5.02 | 3.74E−06 | 0.55 | C8orf34 | 3.38 | 3.54E−03 | 0.73 | MRPL23 | 2.39 | 6.09E−02 | 0.56 |
| RPL32 | 10.04 | 2.20E−22 | 0.9 | COL5A1 | 5.02 | 3.74E−06 | 0.53 | SPARCL1 | 3.38 | 3.53E−03 | 0.64 | HINT2 | 2.39 | 6.09E−02 | 0.53 |
| SUPT6H | 10.03 | 2.40E−22 | 0.69 | FSTL1 | 5.02 | 3.84E−06 | 0.5 | ISYNA1 | 3.38 | 3.45E−03 | 0.53 | TMEM143 | 2.39 | 6.16E−02 | 0.38 |
| EZR | 10.02 | 2.59E−22 | 0.32 | EXOC3L4 | 5.01 | 4.00E−06 | 0.61 | DYNC111 | 3.38 | 3.52E−03 | 0.52 | IRAK1BP1 | 2.39 | 6.14E−02 | 0.29 |
| CTIF | 10.01 | 2.80E−22 | 0.97 | RNF217 | 5.01 | 4.00E−06 | 0.44 | DHDDS | 3.38 | 3.53E−03 | 0.37 | MAMDC2-AS1 | 2.39 | 6.19E−02 | 0.29 |
| FNDC3B | 10.01 | 2.91E−22 | 0.11 | NFIX | 5.01 | 3.93E−06 | 0.34 | KRTCAP3 | 3.38 | 3.55E−03 | 0.29 | ULK2 | 2.39 | 6.09E−02 | 0.25 |
| PLXNA1 | 10 | 3.05E−22 | 1.02 | DYSF | 5 | 4.27E−06 | 1.49 | EIF3M | 3.38 | 3.48E−03 | 0.24 | NOL11 | 2.39 | 6.09E−02 | 0.21 |
| PHLDB2 | 9.98 | 3.87E−22 | 0.88 | PIGR | 5 | 4.21E−06 | 1.22 | POLR2J | 3.37 | 3.67E−03 | 0.79 | CEP83 | 2.39 | 6.16E−02 | 0.2 |
| ABLIM1 | 9.98 | 4.05E−22 | 0.46 | GEM | 5 | 4.16E−06 | 0.97 | DDT | 3.37 | 3.64E−03 | 0.57 | THOC6 | 2.38 | 6.33E−02 | 0.65 |
| NBEAL1 | 9.97 | 4.14E−22 | 0.29 | NDST1 | 5 | 4.22E−06 | 0.88 | HEG1 | 3.37 | 3.67E−03 | 0.53 | GMPR | 2.38 | 6.35E−02 | 0.62 |
| RPL11 | 9.95 | 5.32E−22 | 0.83 | C19orf70 | 5 | 4.10E−06 | 0.78 | ARHGEF17 | 3.37 | 3.58E−03 | 0.51 | SPHK2 | 2.38 | 6.34E−02 | 0.53 |
| RIN2 | 9.95 | 5.36E−22 | 0.5 | NPC2 | 5 | 4.26E−06 | 0.76 | IDH2 | 3.37 | 3.65E−03 | 0.51 | PRPF19 | 2.38 | 6.23E−02 | 0.52 |
| ARL6IP4 | 9.91 | 8.07E−22 | 0.82 | ANKLE2 | 5 | 4.21E−06 | 0.31 | SLC22A15 | 3.37 | 3.56E−03 | 0.44 | ZNF701 | 2.38 | 6.28E−02 | 0.28 |
| UQCRQ | 9.88 | 1.00E−21 | 1.43 | SOSTDC1 | 4.99 | 4.48E−06 | 5.26 | GYS1 | 3.37 | 3.57E−03 | 0.32 | BIN1 | 2.38 | 6.33E−02 | 0.27 |
| RPL24 | 9.88 | 1.02E−21 | 1.02 | AC010967.2 | 4.99 | 4.29E−06 | 3.18 | NCEH1 | 3.37 | 3.64E−03 | 0.25 | SLC15A4 | 2.38 | 6.23E−02 | 0.21 |
| TSC22D3 | 9.88 | 1.01E−21 | 0.55 | OAT | 4.99 | 4.40E−06 | 0.64 | SLC25A27 | 3.36 | 3.72E−03 | 0.65 | LSR | 2.38 | 6.24E−02 | 0.21 |
| TALDO1 | 9.86 | 1.21E−21 | 1.11 | METAP2 | 4.99 | 4.40E−06 | 0.4 | BET1L | 3.36 | 3.67E−03 | 0.47 | PTS | 2.38 | 6.30E−02 | 0.2 |
| DAPK3 | 9.83 | 1.77E−21 | 0.98 | KIZ-AS1 | 4.98 | 4.63E−06 | 1.12 | LEMD2 | 3.36 | 3.70E−03 | 0.38 | ZBTB4 | 2.38 | 6.25E−02 | 0.18 |
| PAPSS2 | 9.79 | 2.61E−21 | 1.32 | KCNMB4 | 4.98 | 4.52E−06 | 0.67 | JADE2 | 3.36 | 3.68E−03 | 0.32 | KLF9 | 2.38 | 6.26E−02 | 0.15 |
| CNTNAP2 | 9.79 | 2.57E−21 | 1.09 | PDIA6 | 4.98 | 4.54E−06 | 0.53 | CARS | 3.36 | 3.75E−03 | 0.25 | FAM214B | 2.38 | 6.30E−02 | 0.12 |
| VAMP8 | 9.77 | 2.94E−21 | 1.02 | MRPS26 | 4.97 | 4.74E−06 | 0.93 | PEF1 | 3.35 | 3.87E−03 | 0.76 | EFHD1 | 2.37 | 6.39E−02 | 1.49 |
| CDK14 | 9.77 | 3.17E−21 | 0.47 | RABL2A | 4.97 | 4.89E−06 | 0.77 | TNNT1 | 3.35 | 3.86E−03 | 0.73 | AC0091204 | 2.37 | 6.48E−02 | 1.49 |
| FBN1 | 9.7 | 5.96E−21 | 1.36 | DIRC2 | 4.97 | 4.86E−06 | 0.5 | TFF2 | 3.35 | 3.93E−03 | 0.34 | NPIPA5 | 2.37 | 6.48E−02 | 1.48 |
| PLA2G16 | 9.7 | 6.16E−21 | 0.7 | DNMT3 A | 4.97 | 4.91E−06 | 0.39 | ZFP36 | 3.35 | 3.80E−03 | 0.33 | CD81-AS1 | 2.37 | 6.47E−02 | 1.36 |
| HSPBAP1 | 9.68 | 7.10E−21 | 1.03 | FOXRED1 | 4.96 | 5.02E−06 | 0.77 | GSDMD | 3.35 | 3.88E−03 | 0.23 | DNAI1 | 2.37 | 6.39E−02 | 1.32 |
| P4HB | 9.68 | 7.35E−21 | 0.69 | SNRPD3 | 4.96 | 5.04E−06 | 0.74 | SCARF2 | 3.34 | 4.03E−03 | 2.38 | CTTNBP2 | 2.37 | 6.51E−02 | 0.87 |
| ZNF609 | 9.63 | 1.21E−20 | 0.35 | PDIA4 | 4.96 | 5.02E−06 | 0.61 | POLR2I | 3.34 | 4.03E−03 | 1.77 | ALDH1L2 | 2.37 | 6.44E−02 | 0.79 |
| CTGF | 9.62 | 1.32E−20 | 0.84 | ZNF350-AS1 | 4.95 | 5.34E−06 | 1.9 | NBAT1 | 3.34 | 4.01E−03 | 1.18 | MEF2C-AS1 | 2.37 | 6.40E−02 | 0.67 |
| SEC61A1 | 9.59 | 1.73E−20 | 0.73 | DSCAML1 | 4.95 | 5.23E−06 | 1.28 | RTP4 | 3.34 | 4.03E−03 | 1.15 | MR1 | 2.37 | 6.43E−02 | 0.51 |
| CTTN | 9.59 | 1.71E−20 | 0.39 | SDCBP2-AS1 | 4.95 | 5.42E−06 | 0.66 | ZNF850 | 3.34 | 3.95E−03 | 0.78 | APIS1 | 2.37 | 6.45E−02 | 0.43 |
| HSP90B1 | 9.58 | 1.95E−20 | 0.81 | BAIAP2 | 4.95 | 5.26E−06 | 0.35 | ZNF493 | 3.34 | 3.94E−03 | 0.76 | JRKL | 2.37 | 6.40E−02 | 0.41 |
| RPLP0 | 9.57 | 2.15E−20 | 0.66 | CAPN3 | 4.94 | 5.69E−06 | 4.38 | TELO2 | 3.34 | 3.94E−03 | 0.31 | PPIE | 2.37 | 6.42E−02 | 0.34 |
| MMP23B | 9.53 | 3.10E−20 | 1.35 | SLC5A1 | 4.94 | 5.57E−06 | 0.79 | PIN1 | 3.33 | 4.20E−03 | 0.53 | DNAJC8 | 2.37 | 6.40E−02 | 0.33 |
| SH3BGRL3 | 9.51 | 3.57E−20 | 1.11 | SERPINH1 | 4.94 | 5.48E−06 | 0.78 | FYTTD1 | 3.33 | 4.21E−03 | 0.37 | AP2A1 | 2.37 | 6.39E−02 | 0.28 |
| LRSAM1 | 9.5 | 3.99E−20 | 0.68 | ZHX3 | 4.94 | 5.60E−06 | 0.52 | PPM1L | 3.33 | 4.19E−03 | 0.35 | ZNF12 | 2.37 | 6.50E−02 | 0.2 |
| CD63 | 9.49 | 4.30E−20 | 0.83 | VPS33B | 4.93 | 6.00E−06 | 3.18 | RRP7A | 3.33 | 4.07E−03 | 0.34 | NDUFV1 | 2.37 | 6.46E−02 | 0.15 |
| IFIT3 | 9.47 | 5.24E−20 | 2.45 | ATF5 | 4.93 | 6.02E−06 | 1.61 | PEA15 | 3.32 | 4.32E−03 | 0.78 | FAM153B | 2.36 | 6.55E−02 | 2.26 |
| NMUR2 | 9.47 | 5.39E−20 | 1.91 | KDM2B | 4.93 | 5.93E−06 | 0.45 | SYNC | 3.32 | 4.31E−03 | 0.63 | MILR1 | 2.36 | 6.56E−02 | 1.07 |
| MFSD14A | 9.46 | 5.86E−20 | 2.42 | BCAR1 | 4.93 | 6.02E−06 | 0.37 | RANBP1 | 3.32 | 4.34E−03 | 0.47 | DNAJC30 | 2.36 | 6.64E−02 | 1.04 |
| ZNF529-AS1 | 9.46 | 5.86E−20 | 1.15 | ABCF2 | 4.92 | 6.17E−06 | 3.11 | PCSK7 | 3.32 | 4.28E−03 | 0.2 | POLE3 | 2.36 | 6.62E−02 | 0.63 |
| ABCA7 | 9.46 | 5.97E−20 | 0.7 | VAT1 | 4.92 | 6.04E−06 | 1.26 | LINC01252 | 3.31 | 4.36E−03 | 0.72 | KIF9 | 2.36 | 6.54E−02 | 0.37 |
| SEC24D | 9.44 | 7.15E−20 | 0.62 | NCBP2-AS2 | 4.92 | 6.26E−06 | 1.14 | TOP3B | 3.31 | 4.37E−03 | 0.68 | CNOT3 | 2.36 | 6.57E−02 | 0.32 |
| CST6 | 9.43 | 8.01E−20 | 3.32 | TCTN1 | 4.92 | 6.31E−06 | 1.11 | GRAMD4 | 3.31 | 4.35E−03 | 0.38 | CIT | 2.36 | 6.52E−02 | 0.24 |
| HIST1H2BB | 9.42 | 8.58E−20 | 2.58 | PDZK1IP1 | 4.92 | 6.17E−06 | 0.85 | ME1 | 3.31 | 4.48E−03 | 0.36 | RTKN | 2.36 | 6.57E−02 | 0.19 |
| RERG | 9.42 | 8.87E−20 | 0.95 | ECH1 | 4.92 | 6.26E−06 | 0.79 | PTPN3 | 3.31 | 4.50E−03 | 0.22 | REL | 2.36 | 6.61E−02 | 0.12 |
| RPS4X | 9.37 | 1.38E−19 | 0.85 | LAPTM4A | 4.92 | 6.13E−06 | 0.61 | HSPA9 | 3.31 | 4.35E−03 | 0.21 | PRX | 2.35 | 6.77E−02 | 1.6 |
| CMIP | 9.37 | 1.37E−19 | 0.35 | CYHR1 | 4.92 | 6.05E−06 | 0.45 | ZNF474 | 3.3 | 4.53E−03 | 5.18 | RUNX3 | 2.35 | 6.68E−02 | 1.48 |
| ZNF37A | 9.36 | 1.54E−19 | 3.77 | NSFL1C | 4.92 | 6.05E−06 | 0.45 | AC005281.1 | 3.3 | 4.55E−03 | 3.19 | AC062017.1 | 2.35 | 6.82E−02 | 1.03 |
| NUPR1 | 9.34 | 1.73E−19 | 2.18 | ANKS1B | 4.91 | 6.59E−06 | 1.11 | CDC42EP1 | 3.3 | 4.53E−03 | 0.49 | POP4 | 2.35 | 6.68E−02 | 0.9 |
| TNC | 9.34 | 1.83E−19 | 1.69 | AES | 4.91 | 6.45E−06 | 0.4 | DUSP5 | 3.3 | 4.54E−03 | 0.35 | CPE | 2.35 | 6.79E−02 | 0.64 |
| SEC61B | 9.31 | 2.39E−19 | 0.98 | ST14 | 4.91 | 6.35E−06 | 0.37 | PSMC1 | 3.3 | 4.55E−03 | 0.31 | MXRA8 | 2.35 | 6.67E−02 | 0.55 |
| CKAP4 | 9.29 | 2.87E−19 | 1.59 | HM13 | 4.91 | 6.45E−06 | 0.31 | SYT17 | 3.3 | 4.61E−03 | 0.25 | VMA21 | 2.35 | 6.74E−02 | 0.46 |
| B2M | 9.29 | 2.87E−19 | 0.52 | C2orf81 | 4.9 | 6.92E−06 | 4.33 | ZNF629 | 3.29 | 4.70E−03 | 1.14 | MEGF8 | 2.35 | 6.73E−02 | 0.36 |
| MRPL41 | 9.28 | 3.18E−19 | 1.17 | NRXN1 | 4.9 | 6.71E−06 | 1.4 | ALG3 | 3.29 | 4.68E−03 | 0.67 | TMEM230 | 2.35 | 6.70E−02 | 0.27 |
| RSPH3 | 9.28 | 3.06E−19 | 0.88 | YWHAB | 4.9 | 6.75E−06 | 0.45 | CRELD2 | 3.29 | 4.71E−03 | 0.54 | PRSS22 | 2.35 | 6.69E−02 | 0.15 |
| ZNRF3 | 9.28 | 3.14E−19 | 0.7 | KIAA1211L | 4.9 | 6.82E−06 | 0.38 | TPST2 | 3.29 | 4.66E−03 | 0.47 | PLBD1 | 2.35 | 6.79E−02 | 0.13 |
| RPL30 | 9.27 | 3.50E−19 | 0.79 | PLD3 | 4.9 | 6.88E−06 | 0.38 | DUSP3 | 3.29 | 4.68E−03 | 0.4 | AC010976.2 | 2.34 | 6.99E−02 | 1.84 |
| RPS24 | 9.24 | 4.33E−19 | 0.59 | JMJD8 | 4.89 | 7.05E−06 | 0.97 | TTF1 | 3.29 | 4.76E−03 | 0.27 | RNF212B | 2.34 | 6.89E−02 | 1.29 |
| PITPNC1 | 9.24 | 4.55E−19 | 0.39 | PLA2G12A | 4.89 | 7.24E−06 | 0.84 | EIF3B | 3.29 | 4.71E−03 | 0.24 | ACOXL | 2.34 | 6.89E−02 | 0.8 |
| LVRN | 9.22 | 5.64E−19 | 3.86 | TBC1D20 | 4.89 | 6.99E−06 | 0.71 | SMOC2 | 3.28 | 4.86E−03 | 0.83 | BST2 | 2.34 | 6.86E−02 | 0.57 |
| TBC1D2 | 9.2 | 6.72E−19 | 0.7 | ALDH7A1 | 4.89 | 6.99E−06 | 0.43 | SSPN | 3.28 | 4.90E−03 | 0.82 | NDUFA8 | 2.34 | 6.92E−02 | 0.53 |
| TPM4 | 9.19 | 7.02E−19 | 0.44 | CACNA2D3 | 4.88 | 7.60E−06 | 1.09 | MRPL50 | 3.28 | 4.87E−03 | 0.74 | HSPBP1 | 2.34 | 6.88E−02 | 0.44 |
| WDFY1 | 9.18 | 7.44E−19 | 0.59 | MRPS11 | 4.88 | 7.56E−06 | 0.78 | YIF1B | 3.28 | 4.84E−03 | 0.59 | NDUFA4 | 2.34 | 6.86E−02 | 0.34 |
| TTC7A | 9.16 | 9.55E−19 | 0.57 | TRAF7 | 4.88 | 7.60E−06 | 0.63 | CUL7 | 3.28 | 4.97E−03 | 0.59 | TOR1B | 2.34 | 6.84E−02 | 0.32 |
| DPYSL3 | 9.15 | 1.02E−18 | 1.13 | HLA-E | 4.88 | 7.63E−06 | 0.43 | ARFRP1 | 3.28 | 4.87E−03 | 0.34 | CYBRD1 | 2.34 | 6.98E−02 | 0.24 |
| HERC6 | 9.15 | 9.98E−19 | 1.05 | RAB1B | 4.87 | 7.72E−06 | 0.77 | COL5A2 | 3.28 | 4.84E−03 | 0.17 | CTBP1 | 2.34 | 6.85E−02 | 0.21 |
| RPS27 | 9.15 | 1.00E−18 | 0.92 | ZNF345 | 4.87 | 8.03E−06 | 0.58 | RRP36 | 3.27 | 5.06E−03 | 0.42 | TIMM23B | 2.34 | 6.88E−02 | 0.16 |
| IL32 | 9.13 | 1.24E−18 | 1.01 | SCYL2 | 4.87 | 7.83E−06 | 0.34 | PSMA4 | 3.27 | 5.01E−03 | 0.34 | PARN | 2.34 | 6.97E−02 | 0.15 |
| VARS2 | 9.1 | 1.62E−18 | 1.5 | LINC00665 | 4.86 | 8.17E−06 | 1.16 | MAP3K14 | 3.27 | 5.09E−03 | 0.32 | AC005253.2 | 2.33 | 7.09E−02 | 2.07 |
| ITGBL1 | 9.1 | 1.55E−18 | 1.03 | SUMO3 | 4.86 | 8.20E−06 | 0.91 | TRMT1 | 3.27 | 5.00E−03 | 0.26 | CUX2 | 2.33 | 7.00E−02 | 1.28 |
| SLC9C1 | 9.09 | 1.80E−18 | 0.86 | CMTR1 | 4.86 | 8.16E−06 | 0.67 | ABCC8 | 3.26 | 5.31E−03 | 1.98 | DXO | 2.33 | 7.01E−02 | 1.05 |
| SYNPO2 | 9.07 | 2.11E−18 | 1.6 | B9D1 | 4.85 | 8.74E−06 | 1.12 | HECW1 | 3.26 | 5.26E−03 | 1.04 | DNAJC27-AS1 | 2.33 | 7.05E−02 | 0.77 |
| TRIM5 | 9.07 | 2.13E−18 | 1.36 | LAMTOR5 | 4.85 | 8.80E−06 | 0.83 | SDF2L1 | 3.26 | 5.18E−03 | 0.98 | CRAT | 2.33 | 7.12E−02 | 0.54 |
| DTX3L | 9.06 | 2.39E−18 | 1.06 | SLIT2 | 4.85 | 8.49E−06 | 0.47 | PMAIP1 | 3.26 | 5.19E−03 | 0.69 | SF3A1 | 2.33 | 7.01E−02 | 0.52 |
| USP31 | 9.04 | 2.67E−18 | 0.78 | LAMC2 | 4.85 | 8.51E−06 | 0.29 | ARHGEF26 | 3.26 | 5.21E−03 | 0.65 | SETD1A | 2.33 | 7.11E−02 | 0.43 |
| ROM01 | 9.03 | 2.93E−18 | 1.73 | POLD4 | 4.84 | 8.86E−06 | 1.33 | ISCU | 3.26 | 5.24E−03 | 0.63 | AVIL | 2.33 | 7.06E−02 | 0.3 |
| ZNF708 | 9.03 | 3.00E−18 | 0.83 | RTF1 | 4.84 | 9.08E−06 | 0.45 | ZNF281 | 3.26 | 5.30E−03 | 0.54 | DUSP23 | 2.33 | 7.06E−02 | 0.28 |
| FILIP1L | 9.03 | 3.12E−18 | 0.77 | EEF2 | 4.84 | 9.25E−06 | 0.33 | HDAC10 | 3.26 | 5.29E−03 | 0.39 | C1orf54 | 2.33 | 7.05E−02 | 0.26 |
| MRC2 | 9.01 | 3.47E−18 | 1.19 | BAD | 4.83 | 9.62E−06 | 0.99 | TMEM30A | 3.26 | 5.27E−03 | 0.35 | ATP13A1 | 2.33 | 7.11E−02 | 0.24 |
| SRPK1 | 9.01 | 3.66E−18 | 0.52 | PSMB8 | 4.83 | 9.63E−06 | 0.94 | CSNK2A1 | 3.26 | 5.26E−03 | 0.2 | HSF2BP | 2.32 | 7.24E−02 | 0.66 |
| SEZ6L2 | 9 | 4.00E−18 | 1.45 | SLC31A1 | 4.83 | 9.69E−06 | 0.71 | AGAP1 | 3.26 | 5.26E−03 | 0.07 | HPN | 2.32 | 7.23E−02 | 0.64 |
| PDE11A | 8.98 | 4.53E−18 | 2.25 | PSMD3 | 4.83 | 9.29E−06 | 0.53 | ZNF358 | 3.25 | 5.44E−03 | 1.09 | SLC38A7 | 2.32 | 7.20E−02 | 0.59 |
| PEBP1 | 8.98 | 4.86E−18 | 1.2 | ASPSCR1 | 4.83 | 9.64E−06 | 0.48 | CPVL | 3.25 | 5.38E−03 | 1.08 | STS | 2.32 | 7.27E−02 | 0.52 |
| EEF1D | 8.98 | 4.55E−18 | 0.64 | ANKRD11 | 4.83 | 9.56E−06 | 0.14 | GTF2H2 | 3.25 | 5.49E−03 | 0.48 | TM2D2 | 2.32 | 7.30E−02 | 0.52 |
| CCDC85B | 8.94 | 6.45E−18 | 1.7 | TNNC2 | 4.82 | 9.94E−06 | 2.9 | BTN3A2 | 3.25 | 5.42E−03 | 0.46 | CDKNIC | 2.32 | 7.31E−02 | 0.41 |
| ATG12 | 8.94 | 6.95E−18 | 0.61 | SOD3 | 4.82 | 1.00E−05 | 2.87 | FAM120AOS | 3.25 | 5.38E−03 | 0.42 | ACTR6 | 2.32 | 7.22E−02 | 0.4 |
| TFDP2 | 8.91 | 8.57E−18 | 0.46 | ELOF1 | 4.82 | 1.00E−05 | 0.93 | NLGN1 | 3.25 | 5.39E−03 | 0.26 | AK8 | 2.32 | 7.30E−02 | 0.3 |
| NNMT | 8.9 | 9.40E−18 | 0.93 | PIGM | 4.81 | 1.07E−05 | 3.33 | SEC16A | 3.25 | 5.42E−03 | 0.19 | KMT2D | 2.32 | 7.24E−02 | 0.18 |
| ZNF611 | 8.9 | 9.60E−18 | 0.68 | GPM6A | 4.8 | 1.10E−05 | 1.52 | CRIP1 | 3.24 | 5.54E−03 | 2.37 | WWTR1 | 2.32 | 7.25E−02 | 0.07 |
| HSD17B3 | 8.89 | 1.02E−17 | 4.69 | FSTL3 | 4.8 | 1.11E−05 | 0.43 | SEMA3F-AS1 | 3.24 | 5.50E−03 | 1.47 | NXF3 | 2.31 | 7.47E−02 | 1.47 |
| C19orf53 | 8.89 | 1.02E−17 | 1.61 | CARHSP1 | 4.8 | 1.12E−05 | 0.43 | ALK | 3.24 | 5.59E−03 | 1.11 | ANAPC15 | 2.31 | 7.39E−02 | 0.96 |
| HES4 | 8.89 | 1.06E−17 | 0.79 | EPS15L1 | 4.8 | 1.09E−05 | 0.36 | LRRC4C | 3.24 | 5.60E−03 | 0.52 | USH2A | 2.31 | 7.40E−02 | 0.96 |
| TPI1 | 8.88 | 1.08E−17 | 0.83 | NAA50 | 4.8 | 1.11E−05 | 0.34 | NAA20 | 3.24 | 5.65E−03 | 0.41 | MALRD1 | 2.31 | 7.36E−02 | 0.72 |
| GPX4 | 8.88 | 1.16E−17 | 0.64 | MTHFD1L | 4.8 | 1.10E−05 | 0.24 | CCDC88C | 3.24 | 5.58E−03 | 0.28 | JDP2 | 2.31 | 7.46E−02 | 0.43 |
| DNAJB11 | 8.87 | 1.21E−17 | 1.61 | ACAT1 | 4.79 | 1.17E−05 | 0.78 | LMF2 | 3.24 | 5.65E−03 | 0.27 | ANKDD1B | 2.31 | 7.37E−02 | 0.42 |
| LINC00685 | 8.84 | 1.58E−17 | 1.39 | LGALS9 | 4.78 | 1.19E−05 | 0.64 | ZCWPW2 | 3.23 | 5.69E−03 | 2.86 | LRCH4 | 2.31 | 7.38E−02 | 0.34 |
| MMP14 | 8.83 | 1.78E−17 | 0.9 | SPSB1 | 4.78 | 1.18E−05 | 0.58 | UNC45A | 3.23 | 5.68E−03 | 0.32 | BTBD19 | 2.31 | 7.45E−02 | 0.34 |
| VCAN | 8.82 | 1.89E−17 | 0.66 | NUDC | 4.78 | 1.22E−05 | 0.49 | DAB1 | 3.23 | 5.80E−03 | 0.23 | NR1H2 | 2.31 | 7.40E−02 | 0.31 |
| ITGB5 | 8.81 | 2.06E−17 | 0.48 | DYNLRB1 | 4.78 | 1.20E−05 | 0.48 | TMEM246 | 3.22 | 5.96E−03 | 0.8 | EPX | 2.3 | 7.59E−02 | 2.36 |
| QTRT1 | 8.78 | 2.77E−17 | 0.72 | CANX | 4.78 | 1.19E−05 | 0.37 | VARS | 3.22 | 5.86E−03 | 0.56 | GPR1 | 2.3 | 7.62E−02 | 1.76 |
| SDC1 | 8.77 | 2.93E−17 | 0.81 | C2orf54 | 4.77 | 1.26E−05 | 1.68 | PIH1D1 | 3.22 | 6.04E−03 | 0.37 | CCBE1 | 2.3 | 7.55E−02 | 1.19 |
| ATP6V1A | 8.77 | 3.04E−17 | 0.71 | CAMKK1 | 4.77 | 1.26E−05 | 1.09 | LINC00667 | 3.22 | 5.89E−03 | 0.36 | ZNF275 | 2.3 | 7.66E−02 | 0.89 |
| B3GALT5 | 8.77 | 3.09E−17 | 0.67 | SCYL1 | 4.77 | 1.27E−05 | 0.72 | ACLY | 3.22 | 5.97E−03 | 0.32 | TMEM92-AS1 | 2.3 | 7.55E−02 | 0.63 |
| WNT5B | 8.74 | 3.81E−17 | 0.86 | NUTF2 | 4.77 | 1.28E−05 | 0.64 | MYO9B | 3.22 | 6.00E−03 | 0.19 | MC1R | 2.3 | 7.66E−02 | 0.58 |
| PML | 8.73 | 4.08E−17 | 0.71 | TSPAN4 | 4.77 | 1.25E−05 | 0.52 | TSKU | 3.21 | 6.24E−03 | 1.39 | SIPA1 | 2.3 | 7.59E−02 | 0.49 |
| NHSL2 | 8.72 | 4.58E−17 | 1.78 | PPP2RIA | 4.77 | 1.28E−05 | 0.51 | SF3B5 | 3.21 | 6.06E−03 | 0.67 | CDCA7L | 2.3 | 7.65E−02 | 0.49 |
| RPL7A | 8.72 | 4.45E−17 | 0.75 | SLC3A2 | 4.77 | 1.26E−05 | 0.43 | NDUFC1 | 3.21 | 6.07E−03 | 0.63 | CCDC97 | 2.3 | 7.56E−02 | 0.47 |
| RPL21 | 8.71 | 4.86E−17 | 0.85 | SPCS2 | 4.77 | 1.25E−05 | 0.41 | LRGUK | 3.21 | 6.24E−03 | 0.54 | BUD31 | 2.3 | 7.56E−02 | 0.46 |
| TCF25 | 8.71 | 5.02E−17 | 0.38 | TMC8 | 4.76 | 1.33E−05 | 1.11 | POLD2 | 3.21 | 6.07E−03 | 0.46 | RSL24D1 | 2.3 | 7.59E−02 | 0.41 |
| NTN4 | 8.7 | 5.36E−17 | 0.57 | BCO2 | 4.76 | 1.31E−05 | 0.98 | MPZL2 | 3.21 | 6.07E−03 | 0.37 | CACNB3 | 2.3 | 7.68E−02 | 0.35 |
| PRKCI | 8.67 | 7.35E−17 | 0.42 | LINC00578 | 4.76 | 1.33E−05 | 0.96 | CARD19 | 3.21 | 6.16E−03 | 0.33 | CDON | 2.3 | 7.55E−02 | 0.26 |
| FAM50A | 8.66 | 7.35E−17 | 0.86 | SFN | 4.76 | 1.34E−05 | 0.61 | FBXW5 | 3.21 | 6.14E−03 | 0.23 | ZNF618 | 2.3 | 7.57E−02 | 0.21 |
| TNS3 | 8.65 | 8.40E−17 | 0.42 | AC026202.3 | 4.76 | 1.31E−05 | 0.54 | B3GALT6 | 3.2 | 6.43E−03 | 1.58 | PEPD | 2.3 | 7.57E−02 | 0.2 |
| TMED2 | 8.64 | 9.35E−17 | 1.33 | PTPRH | 4.76 | 1.34E−05 | 0.5 | ASIP | 3.2 | 6.44E−03 | 1.24 | EME2 | 2.3 | 7.68E−02 | 0.2 |
| SYNE1 | 8.64 | 9.06E−17 | 0.82 | DPP7 | 4.76 | 1.30E−05 | 0.4 | PDZD7 | 3.2 | 6.42E−03 | 1.13 | ZNF532 | 2.3 | 7.58E−02 | 0.15 |
| ZNF565 | 8.64 | 9.49E−17 | 0.63 | RAB7A | 4.76 | 1.31E−05 | 0.21 | DCXR | 3.2 | 6.42E−03 | 0.98 | EEF1B2 | 2.3 | 7.66E−02 | 0.09 |
| TTLL7 | 8.63 | 9.82E−17 | 1.59 | TSR3 | 4.75 | 1.38E−05 | 1.1 | SGIP1 | 3.2 | 6.42E−03 | 0.77 | CEACAM8 | 2.29 | 7.72E−02 | 2.68 |
| KCNK6 | 8.62 | 1.11E−16 | 0.79 | COMMD2 | 4.75 | 1.38E−05 | 0.69 | SULT1A1 | 3.2 | 6.37E−03 | 0.58 | MDGA2 | 2.29 | 7.87E−02 | 1.23 |
| WIPI1 | 8.61 | 1.15E−16 | 0.83 | ALDH3B1 | 4.75 | 1.42E−05 | 0.59 | PLS3 | 3.2 | 6.38E−03 | 0.23 | NAALADL2-AS2 | 2.29 | 7.71E−02 | 0.5 |
| SPAG1 | 8.61 | 1.20E−16 | 0.79 | BFAR | 4.75 | 1.36E−05 | 0.46 | SRGAP1 | 3.2 | 6.41E−03 | 0.15 | PYROXD2 | 2.29 | 7.75E−02 | 0.41 |
| UBALD2 | 8.6 | 1.26E−16 | 0.68 | COPRS | 4.74 | 1.46E−05 | 1.23 | CDC42EP2 | 3.19 | 6.66E−03 | 1.36 | FBXL15 | 2.29 | 7.85E−02 | 0.39 |
| RPL3 | 8.59 | 1.36E−16 | 0.67 | MEF2C | 4.74 | 1.46E−05 | 0.79 | ZNF682 | 3.19 | 6.47E−03 | 0.99 | NDUFAB1 | 2.29 | 7.80E−02 | 0.28 |
| ZNF432 | 8.58 | 1.47E−16 | 0.75 | TMEM159 | 4.74 | 1.44E−05 | 0.56 | LINC00882 | 3.19 | 6.51E−03 | 0.71 | ENO1 | 2.29 | 7.78E−02 | 0.17 |
| CLUAP1 | 8.57 | 1.72E−16 | 1.08 | PLIN3 | 4.74 | 1.46E−05 | 0.48 | C11orf24 | 3.19 | 6.56E−03 | 0.63 | ATP2C1 | 2.29 | 7.74E−02 | 0.12 |
| SNHG25 | 8.55 | 1.98E−16 | 0.66 | MT-ND2 | 4.74 | 1.44E−05 | 0.45 | EBF4 | 3.19 | 6.57E−03 | 0.6 | SRGAP3 | 2.29 | 7.74E−02 | 0.12 |
| CHRM2 | 8.54 | 2.21E−16 | 1.99 | CDK18 | 4.74 | 1.44E−05 | 0.38 | UQCC2 | 3.19 | 6.53E−03 | 0.54 | IFIT5 | 2.28 | 7.88E−02 | 1.16 |
| KDM4A | 8.53 | 2.30E−16 | 3.79 | PITPNA | 4.74 | 1.42E−05 | 0.35 | RHOF | 3.19 | 6.66E−03 | 0.47 | RABIF | 2.28 | 7.96E−02 | 0.74 |
| PDXK | 8.53 | 2.30E−16 | 0.44 | GLG1 | 4.74 | 1.48E−05 | 0.18 | BRAP | 3.19 | 6.60E−03 | 0.32 | C11orf68 | 2.28 | 7.92E−02 | 0.73 |
| ZER1 | 8.52 | 2.52E−16 | 0.65 | USP18 | 4.73 | 1.50E−05 | 1.42 | CEP164 | 3.19 | 6.47E−03 | 0.3 | MRPS33 | 2.28 | 7.94E−02 | 0.72 |
| CD74 | 8.51 | 2.84E−16 | 1.32 | MDC1 | 4.73 | 1.55E−05 | 0.95 | HERC3 | 3.19 | 6.66E−03 | 0.27 | CDK4 | 2.28 | 7.99E−02 | 0.49 |
| CEACAM6 | 8.51 | 2.76E−16 | 0.79 | PCDH11Y | 4.72 | 1.57E−05 | 1.35 | PPCDC | 3.19 | 6.53E−03 | 0.26 | PMP22 | 2.28 | 7.96E−02 | 0.45 |
| PSMB9 | 8.5 | 3.01E−16 | 1.5 | GLIS2 | 4.72 | 1.56E−05 | 1 | HMGXB4 | 3.19 | 6.56E−03 | 0.23 | ZNF675 | 2.28 | 7.96E−02 | 0.32 |
| USP49 | 8.5 | 3.05E−16 | 0.82 | UQCC3 | 4.72 | 1.58E−05 | 0.94 | ST8SIA6-AS1 | 3.18 | 6.72E−03 | 3.5 | RAB8A | 2.28 | 8.04E−02 | 0.28 |
| RAMP1 | 8.49 | 3.22E−16 | 1.11 | PCAT7 | 4.72 | 1.56E−05 | 0.93 | ZFP30 | 3.18 | 6.70E−03 | 0.45 | KLHL22 | 2.28 | 8.05E−02 | 0.28 |
| AUP1 | 8.47 | 3.78E−16 | 0.81 | TMEM214 | 4.72 | 1.60E−05 | 0.66 | CTNNBIP1 | 3.18 | 6.73E−03 | 0.28 | SSU72 | 2.28 | 7.91E−02 | 0.18 |
| BANF1 | 8.45 | 4.69E−16 | 1.49 | STRAP | 4.72 | 1.63E−05 | 0.44 | ITPR3 | 3.18 | 6.81E−03 | 0.19 | WDPCP | 2.28 | 7.93E−02 | 0.16 |
| SCRN1 | 8.45 | 4.38E−16 | 0.7 | CC2D1A | 4.72 | 1.61E−05 | 0.34 | MAPK12 | 3.17 | 7.10E−03 | 1.05 | MICALL2 | 2.28 | 8.04E−02 | 0.16 |
| RILPL1 | 8.43 | 5.19E−16 | 0.89 | TTR | 4.71 | 1.65E−05 | 2.76 | CYP19A1 | 3.17 | 7.03E−03 | 0.82 | PHF20 | 2.28 | 7.98E−02 | 0.13 |
| GABPB1-AS1 | 8.43 | 5.44E−16 | 0.64 | LINC01242 | 4.71 | 1.66E−05 | 1.72 | DEXI | 3.17 | 6.92E−03 | 0.71 | EHBP1 | 2.28 | 8.04E−02 | 0.03 |
| NUAK1 | 8.4 | 6.83E−16 | 1.3 | IGFBP6 | 4.71 | 1.66E−05 | 0.94 | BGN | 3.17 | 6.98E−03 | 0.7 | AC007091.1 | 2.27 | 8.19E−02 | 3.11 |
| TBCEL | 8.38 | 8.24E−16 | 4.42 | GADD45A | 4.71 | 1.70E−05 | 0.73 | POU2F2 | 3.17 | 6.93E−03 | 0.47 | KDELR3 | 2.27 | 8.17E−02 | 0.69 |
| MARK4 | 8.36 | 9.77E−16 | 0.68 | UBXN1 | 4.71 | 1.69E−05 | 0.56 | TGFB2 | 3.17 | 6.96E−03 | 0.31 | FADS1 | 2.27 | 8.07E−02 | 0.65 |
| PTMA | 8.35 | 1.01E−15 | 0.51 | HMGA2 | 4.71 | 1.66E−05 | 0.42 | BIRC2 | 3.17 | 7.05E−03 | 0.26 | POLE4 | 2.27 | 8.22E−02 | 0.44 |
| DMGDH | 8.34 | 1.12E−15 | 1.68 | RHPN1 | 4.71 | 1.64E−05 | 0.32 | TMED10 | 3.17 | 7.04E−03 | 0.24 | NUDT16 | 2.27 | 8.24E−02 | 0.42 |
| TAF8 | 8.34 | 1.15E−15 | 1.01 | B4GALNT2 | 4.7 | 1.74E−05 | 1.63 | GJA1 | 3.16 | 7.28E−03 | 1.29 | CDC34 | 2.27 | 8.22E−02 | 0.32 |
| STK38 | 8.33 | 1.21E−15 | 0.42 | MAATS1 | 4.7 | 1.79E−05 | 0.98 | DOHH | 3.16 | 7.22E−03 | 1.25 | FBXO16 | 2.27 | 8.20E−02 | 0.29 |
| SRRM2 | 8.33 | 1.20E−15 | 0.39 | NUBP2 | 4.7 | 1.72E−05 | 0.83 | CFAP43 | 3.16 | 7.32E−03 | 0.7 | NOSIP | 2.27 | 8.16E−02 | 0.22 |
| RNF7 | 8.32 | 1.35E−15 | 1.16 | MCC | 4.7 | 1.74E−05 | 0.21 | SVIP | 3.16 | 7.17E−03 | 0.5 | ARPP19 | 2.27 | 8.06E−02 | 0.19 |
| CYR61 | 8.3 | 1.57E−15 | 1.07 | UBXN6 | 4.69 | 1.81E−05 | 0.68 | ARMC2 | 3.16 | 7.31E−03 | 0.36 | PDE8B | 2.26 | 8.27E−02 | 2.29 |
| BSG | 8.3 | 1.58E−15 | 0.85 | TBC1D22B | 4.69 | 1.84E−05 | 0.39 | TRIM22 | 3.16 | 7.26E−03 | 0.34 | SRRM3 | 2.26 | 8.34E−02 | 1.39 |
| SSH1 | 8.29 | 1.72E−15 | 0.75 | LINC01429 | 4.68 | 1.93E−05 | 1.7 | CLPTM1L | 3.16 | 7.13E−03 | 0.31 | STXBP5L | 2.26 | 8.34E−02 | 1.12 |
| COX5B | 8.26 | 2.21E−15 | 1.21 | MED29 | 4.68 | 1.92E−05 | 1.07 | ANAPC16 | 3.16 | 7.22E−03 | 0.21 | FILIP1 | 2.26 | 8.27E−02 | 0.98 |
| OAS2 | 8.25 | 2.41E−15 | 2.34 | BRMS1 | 4.68 | 1.89E−05 | 0.86 | CCDC57 | 3.16 | 7.25E−03 | 0.21 | KLF16 | 2.26 | 8.43E−02 | 0.56 |
| LRP1B | 8.25 | 2.37E−15 | 1.3 | DLC1 | 4.68 | 1.94E−05 | 0.46 | PHGDH | 3.15 | 7.45E−03 | 1.27 | FAM110A | 2.26 | 8.36E−02 | 0.51 |
| ITPKC | 8.25 | 2.37E−15 | 0.6 | LDHB | 4.67 | 1.98E−05 | 1.21 | TMEM222 | 3.15 | 7.46E−03 | 0.66 | PGD | 2.26 | 8.29E−02 | 0.44 |
| FKBP8 | 8.24 | 2.52E−15 | 0.9 | MYO16-AS1 | 4.67 | 2.03E−05 | 0.79 | ATXN7L3B | 3.15 | 7.39E−03 | 0.62 | ACOT8 | 2.26 | 8.28E−02 | 0.33 |
| FAM83A | 8.24 | 2.61E−15 | 0.86 | KMT5C | 4.67 | 1.97E−05 | 0.68 | C19orf48 | 3.15 | 7.55E−03 | 0.45 | PPP1R18 | 2.26 | 8.34E−02 | 0.33 |
| MIR4435-2HG | 8.24 | 2.59E−15 | 0.34 | RGN | 4.66 | 2.06E−05 | 2.44 | FEM1B | 3.15 | 7.55E−03 | 0.41 | ARRDC2 | 2.26 | 8.27E−02 | 0.28 |
| DLG2 | 8.22 | 2.95E−15 | 0.74 | AMOTL1 | 4.66 | 2.07E−05 | 0.54 | LRRC23 | 3.15 | 7.49E−03 | 0.34 | JAG1 | 2.26 | 8.37E−02 | 0.27 |
| RPL36AL | 8.2 | 3.53E−15 | 0.83 | HINT1 | 4.66 | 2.10E−05 | 0.45 | CRY2 | 3.15 | 7.44E−03 | 0.33 | HAX1 | 2.26 | 8.33E−02 | 0.26 |
| CDR2 | 8.2 | 3.48E−15 | 0.35 | SSBP3 | 4.66 | 2.06E−05 | 0.33 | CAPS | 3.15 | 7.41E−03 | 0.3 | SGTA | 2.26 | 8.33E−02 | 0.22 |
| LINC01170 | 8.19 | 3.91E−15 | 2.46 | ZBTB38 | 4.66 | 2.11E−05 | 0.28 | STIP1 | 3.15 | 7.35E−03 | 0.29 | ACAD9 | 2.26 | 8.41E−02 | 0.2 |
| SLCO4A1 | 8.19 | 3.83E−15 | 1.05 | MRGBP | 4.65 | 2.16E−05 | 0.74 | UBE20 | 3.15 | 7.40E−03 | 0.23 | CEP70 | 2.26 | 8.30E−02 | 0.06 |
| CAPNS1 | 8.19 | 4.05E−15 | 0.97 | NACC1 | 4.65 | 2.23E−05 | 0.7 | NAA25 | 3.15 | 7.55E−03 | 0.22 | PODNL1 | 2.25 | 8.63E−02 | 1.64 |
| AGGF1 | 8.17 | 4.74E−15 | 2.91 | AZIN1-AS1 | 4.65 | 2.19E−05 | 0.47 | ITGA4 | 3.14 | 7.71E−03 | 1.57 | CFAP61 | 2.25 | 8.57E−02 | 0.9 |
| MYL12A | 8.17 | 4.69E−15 | 0.67 | CLEC19A | 4.64 | 2.34E−05 | 1.46 | C20orf27 | 3.14 | 7.75E−03 | 1.12 | BAK1 | 2.25 | 8.55E−02 | 0.68 |
| TSPAN13 | 8.16 | 5.00E−15 | 1.11 | TMEM104 | 4.64 | 2.26E−05 | 0.98 | MRPL17 | 3.14 | 7.63E−03 | 0.99 | L3MBTL2 | 2.25 | 8.54E−02 | 0.34 |
| THSD7A | 8.16 | 5.04E−15 | 0.71 | SYNPO | 4.64 | 2.30E−05 | 0.65 | SLC25A22 | 3.14 | 7.70E−03 | 0.83 | TSPAN17 | 2.25 | 8.55E−02 | 0.33 |
| GSTP1 | 8.16 | 5.04E−15 | 0.62 | PRKD2 | 4.64 | 2.34E−05 | 0.34 | FAM89B | 3.14 | 7.61E−03 | 0.82 | RRAS | 2.25 | 8.53E−02 | 0.3 |
| HSPG2 | 8.16 | 4.92E−15 | 0.4 | ATP1B3 | 4.64 | 2.33E−05 | 0.21 | EVA1A | 3.14 | 7.79E−03 | 0.73 | LACTB | 2.25 | 8.62E−02 | 0.27 |
| OGFR | 8.15 | 5.52E−15 | 0.76 | CARMN | 4.63 | 2.38E−05 | 1.3 | GTF2F1 | 3.14 | 7.66E−03 | 0.63 | DPY30 | 2.25 | 8.61E−02 | 0.19 |
| ADM | 8.13 | 6.31E−15 | 1.26 | C19orf24 | 4.63 | 2.37E−05 | 0.95 | AGTRAP | 3.14 | 7.77E−03 | 0.47 | MAPK13 | 2.25 | 8.48E−02 | 0.16 |
| SLC27A4 | 8.13 | 6.32E−15 | 0.98 | LRRC20 | 4.63 | 2.37E−05 | 0.83 | MAP3K6 | 3.14 | 7.72E−03 | 0.42 | ESR2 | 2.25 | 8.62E−02 | 0.14 |
| MT-ND4 | 8.13 | 6.46E−15 | 0.66 | SCNM1 | 4.63 | 2.41E−05 | 0.7 | RANGAP1 | 3.14 | 7.77E−03 | 0.26 | ZNF114 | 2.24 | 8.73E−02 | 1.76 |
| CASC9 | 8.11 | 7.28E−15 | 0.74 | KIAA0930 | 4.63 | 2.38E−05 | 0.66 | PRC1 | 3.13 | 7.89E−03 | 1.59 | LAMP3 | 2.24 | 8.64E−02 | 1.47 |
| GCNT1 | 8.1 | 8.30E−15 | 0.97 | CXorf38 | 4.63 | 2.37E−05 | 0.59 | TOMM22 | 3.13 | 7.93E−03 | 0.95 | ST6GALNAC5 | 2.24 | 8.69E−02 | 0.89 |
| LTBR | 8.1 | 8.06E−15 | 0.57 | DCBLD1 | 4.63 | 2.39E−05 | 0.39 | CCHCR1 | 3.13 | 7.92E−03 | 0.36 | UBL4A | 2.24 | 8.78E−02 | 0.85 |
| SLFNL1 | 8.08 | 9.72E−15 | 1.39 | GPR176 | 4.62 | 2.49E−05 | 1.03 | RNF34 | 3.13 | 7.80E−03 | 0.34 | ZFPL1 | 2.24 | 8.69E−02 | 0.52 |
| HSPA5 | 8.08 | 9.17E−15 | 0.86 | RUVBL2 | 4.62 | 2.50E−05 | 0.77 | STARD3 | 3.13 | 7.93E−03 | 0.28 | TMEM44-AS1 | 2.24 | 8.73E−02 | 0.52 |
| HCFCIR1 | 8.06 | 1.13E−14 | 1.3 | NT5DC3 | 4.62 | 2.47E−05 | 0.61 | TPBG | 3.13 | 8.01E−03 | 0.28 | COMMD6 | 2.24 | 8.69E−02 | 0.42 |
| SIPA1L3 | 8.06 | 1.14E−14 | 0.29 | ARL3 | 4.62 | 2.58E−05 | 0.6 | KIAA0319 | 3.12 | 8.24E−03 | 1.01 | PDZRN3 | 2.24 | 8.80E−02 | 0.36 |
| CACNB2 | 8.04 | 1.26E−14 | 1.03 | LMOD1 | 4.61 | 2.66E−05 | 2.34 | GRK4 | 3.12 | 8.17E−03 | 0.76 | TCP11L2 | 2.24 | 8.82E−02 | 0.27 |
| RPS5 | 8.04 | 1.34E−14 | 0.93 | IDH3G | 4.61 | 2.69E−05 | 0.89 | ZNF703 | 3.12 | 8.16E−03 | 0.6 | MTCL1 | 2.24 | 8.68E−02 | 0.16 |
| NABP1 | 8.04 | 1.33E−14 | 0.75 | YIF1A | 4.61 | 2.67E−05 | 0.73 | MYOM3 | 3.12 | 8.21E−03 | 0.57 | PDE10A | 2.24 | 8.69E−02 | 0.16 |
| MARVELD1 | 8.03 | 1.44E−14 | 1.08 | SP14OL | 4.61 | 2.63E−05 | 0.41 | ZFAND2A | 3.12 | 8.22E−03 | 0.57 | DDX60 | 2.24 | 8.76E−02 | 0.13 |
| SPTAN1 | 8.02 | 1.53E−14 | 0.44 | VTCN1 | 4.6 | 2.76E−05 | 1.42 | EIF3I | 3.12 | 8.15E−03 | 0.48 | KBTBD12 | 2.24 | 8.78E−02 | 0.1 |
| EEPD1 | 8.02 | 1.47E−14 | 0.38 | YDJC | 4.6 | 2.72E−05 | 1.1 | RPL22 | 3.12 | 8.17E−03 | 0.34 | MYOM1 | 2.24 | 8.70E−02 | 0.08 |
| DENND6B | 8.01 | 1.65E−14 | 0.93 | CLPTM1 | 4.6 | 2.71E−05 | 0.51 | SLC17A5 | 3.12 | 8.13E−03 | 0.21 | DNAH8 | 2.23 | 8.92E−02 | 1.35 |
| CLSTN1 | 8.01 | 1.72E−14 | 0.48 | HCG27 | 4.59 | 2.90E−05 | 1.56 | Z838441 | 3.11 | 8.42E−03 | 2.53 | MFSD5 | 2.23 | 8.83E−02 | 1.34 |
| VAV2 | 8.01 | 1.61E−14 | 0.46 | CDIPT | 4.59 | 2.88E−05 | 0.98 | CNN1 | 3.11 | 8.40E−03 | 2.09 | CA11 | 2.23 | 8.99E−02 | 1.03 |
| GOS2 | 8 | 1.83E−14 | 2.32 | NCKAP5L | 4.59 | 2.86E−05 | 0.89 | IGSF5 | 3.11 | 8.51E−03 | 1.4 | KCTD12 | 2.23 | 8.83E−02 | 0.83 |
| TM4SF1-AS1 | 8 | 1.78E−14 | 1.42 | SEMA5A | 4.59 | 2.87E−05 | 0.82 | NCAM2 | 3.11 | 8.41E−03 | 1.05 | SMDT1 | 2.23 | 8.83E−02 | 0.69 |
| RHOD | 8 | 1.77E−14 | 1.09 | YKT6 | 4.59 | 2.87E−05 | 0.77 | NPIPB11 | 3.11 | 8.39E−03 | 0.94 | GTF3C6 | 2.23 | 8.96E−02 | 0.51 |
| TMED9 | 7.99 | 2.00E−14 | 1.19 | LYPLA2 | 4.59 | 2.85E−05 | 0.68 | POLR2C | 3.11 | 8.43E−03 | 0.65 | OSBPL10-AS1 | 2.23 | 8.88E−02 | 0.49 |
| GGPS1 | 7.98 | 2.16E−14 | 3 | KIAA0825 | 4.59 | 2.86E−05 | 0.35 | PRKACA | 3.11 | 8.49E−03 | 0.59 | ALG2 | 2.23 | 8.97E−02 | 0.44 |
| RAB26 | 7.98 | 2.06E−14 | 1.48 | CFAP47 | 4.58 | 3.04E−05 | 1.32 | RASGRP3 | 3.11 | 8.41E−03 | 0.58 | ICAM1 | 2.23 | 8.88E−02 | 0.37 |
| DCDC1 | 7.98 | 2.01E−14 | 1.16 | CDR2L | 4.58 | 3.02E−05 | 0.8 | EHMT2 | 3.11 | 8.48E−03 | 0.39 | PLEKHA4 | 2.23 | 8.94E−02 | 0.32 |
| CAPG | 7.98 | 2.14E−14 | 0.99 | CDKL1 | 4.58 | 3.01E−05 | 0.61 | SIGIRR | 3.11 | 8.49E−03 | 0.26 | DCAF15 | 2.23 | 8.98E−02 | 0.26 |
| SLC16A1-AS1 | 7.98 | 2.01E−14 | 0.95 | DNHD1 | 4.58 | 3.01E−05 | 0.51 | RAB12 | 3.11 | 8.40E−03 | 0.2 | NFE2L3 | 2.23 | 8.83E−02 | 0.25 |
| UBE2I | 7.98 | 2.16E−14 | 0.51 | MYO1E | 4.58 | 3.02E−05 | 0.14 | GLTP | 3.1 | 8.63E−03 | 0.46 | VPS29 | 2.23 | 8.88E−02 | 0.15 |
| TLL1 | 7.97 | 2.16E−14 | 1.94 | HBG2 | 4.57 | 3.10E−05 | 3.68 | C9orf78 | 3.1 | 8.69E−03 | 0.42 | NR2C2AP | 2.22 | 9.12E−02 | 0.72 |
| TBC1D17 | 7.96 | 2.51E−14 | 0.77 | APOBEC2 | 4.57 | 3.10E−05 | 2.43 | TMEM179B | 3.1 | 8.65E−03 | 0.4 | CLU | 2.22 | 9.08E−02 | 0.66 |
| NMT1 | 7.96 | 2.45E−14 | 0.51 | SLC2A1-AS1 | 4.57 | 3.18E−05 | 0.9 | PDE9A | 3.1 | 8.71E−03 | 0.39 | KATNB1 | 2.22 | 9.08E−02 | 0.59 |
| HIP1R | 7.95 | 2.55E−14 | 0.66 | LAMTOR2 | 4.57 | 3.13E−05 | 0.88 | KDSR | 3.1 | 8.61E−03 | 0.38 | TRPT1 | 2.22 | 9.19E−02 | 0.57 |
| MICA | 7.91 | 3.66E−14 | 0.86 | BLVRA | 4.57 | 3.23E−05 | 0.81 | URB1 | 3.1 | 8.65E−03 | 0.31 | ERBB4 | 2.22 | 9.19E−02 | 0.32 |
| SH3PXD2A | 7.91 | 3.51E−14 | 0.45 | FYN | 4.57 | 3.23E−05 | 0.47 | TMC2 | 3.09 | 9.00E−03 | 1.76 | STOML2 | 2.22 | 9.20E−02 | 0.31 |
| PAWR | 7.9 | 4.05E−14 | 0.4 | ADAM19 | 4.57 | 3.23E−05 | 0.45 | SNTG1 | 3.09 | 8.85E−03 | 0.99 | EPPK1 | 2.22 | 9.06E−02 | 0.22 |
| SHARPIN | 7.88 | 4.60E−14 | 0.78 | S100A10 | 4.57 | 3.16E−05 | 0.29 | TGFB1I1 | 3.09 | 8.88E−03 | 0.85 | PITHD1 | 2.22 | 9.19E−02 | 0.21 |
| EFCAB3 | 7.87 | 5.12E−14 | 1.84 | KPNB1 | 4.57 | 3.15E−05 | 0.25 | VPS25 | 3.09 | 9.10E−03 | 0.73 | TLL2 | 2.21 | 9.24E−02 | 1.29 |
| CFH | 7.87 | 4.92E−14 | 0.72 | ANAPC13 | 4.56 | 3.33E−05 | 1.34 | OAZ2 | 3.09 | 9.01E−03 | 0.57 | TIMM10 | 2.21 | 9.41E−02 | 1.02 |
| OAS1 | 7.86 | 5.26E−14 | 1.18 | GABRB3 | 4.56 | 3.27E−05 | 0.91 | MGAM2 | 3.09 | 9.10E−03 | 0.52 | CSMD2 | 2.21 | 9.33E−02 | 0.83 |
| COL4A1 | 7.86 | 5.33E−14 | 1.1 | POLR2F | 4.56 | 3.29E−05 | 0.75 | OBSL1 | 3.09 | 9.04E−03 | 0.44 | FADD | 2.21 | 9.28E−02 | 0.77 |
| NUDT3 | 7.84 | 6.27E−14 | 1.21 | MRPL33 | 4.56 | 3.27E−05 | 0.65 | CCDC92 | 3.09 | 8.84E−03 | 0.31 | PARP2 | 2.21 | 9.41E−02 | 0.49 |
| CHCHD2 | 7.82 | 7.34E−14 | 0.83 | SLC25A43 | 4.56 | 3.33E−05 | 0.35 | ZNF766 | 3.09 | 8.98E−03 | 0.28 | PRRG2 | 2.21 | 9.29E−02 | 0.46 |
| TMEM120B | 7.82 | 7.27E−14 | 0.75 | MRPS21 | 4.56 | 3.33E−05 | 0.33 | TUBB6 | 3.09 | 9.05E−03 | 0.2 | AMZ2 | 2.21 | 9.28E−02 | 0.41 |
| IRF8 | 7.81 | 7.68E−14 | 1.91 | TRPM3 | 4.55 | 3.45E−05 | 0.98 | SGSM1 | 3.08 | 9.35E−03 | 0.75 | MDFI | 2.21 | 9.24E−02 | 0.31 |
| DZIP1L | 7.81 | 7.79E−14 | 1.5 | PLXNB2 | 4.55 | 3.53E−05 | 0.3 | CHTF18 | 3.08 | 9.21E−03 | 0.71 | GAS6 | 2.21 | 9.37E−02 | 0.31 |
| MPG | 7.81 | 8.06E−14 | 0.76 | SNRPF | 4.54 | 3.63E−05 | 0.99 | MRPL27 | 3.08 | 9.18E−03 | 0.43 | EFCAB6 | 2.21 | 9.42E−02 | 0.31 |
| RPS26 | 7.8 | 8.22E−14 | 1.64 | CDC42EP3 | 4.54 | 3.63E−05 | 0.81 | DPF2 | 3.08 | 9.31E−03 | 0.36 | HMCES | 2.21 | 9.42E−02 | 0.29 |
| DMKN | 7.8 | 8.22E−14 | 0.59 | SNHG7 | 4.54 | 3.56E−05 | 0.48 | SAMD9L | 3.08 | 9.34E−03 | 0.32 | SARAF | 2.21 | 9.24E−02 | 0.15 |
| BLVRB | 7.79 | 8.96E−14 | 0.66 | TCF4 | 4.54 | 3.64E−05 | 0.48 | SLFN5 | 3.08 | 9.18E−03 | 0.17 | ZNF791 | 2.21 | 9.27E−02 | 0.15 |
| TMEM241 | 7.79 | 9.30E−14 | 0.48 | DDB1 | 4.54 | 3.62E−05 | 0.38 | CPTP | 3.07 | 9.65E−03 | 1.43 | IQCG | 2.21 | 9.28E−02 | 0.14 |
| MED17 | 7.78 | 1.03E−13 | 2.29 | C1orf50 | 4.53 | 3.79E−05 | 3.27 | B3GALT5-AS1 | 3.07 | 9.51E−03 | 1.32 | EPB41 | 2.21 | 9.42E−02 | 0.13 |
| XRN1 | 7.78 | 1.01E−13 | 0.38 | ZNF880 | 4.53 | 3.79E−05 | 1.9 | TMEM203 | 3.07 | 9.43E−03 | 0.96 | HMGN3 | 2.21 | 9.43E−02 | 0.1 |
| PDLIM4 | 7.77 | 1.08E−13 | 1.97 | TAX1BP3 | 4.53 | 3.77E−05 | 0.98 | GRWD1 | 3.07 | 9.54E−03 | 0.92 | B4GALNT1 | 2.2 | 9.60E−02 | 2.68 |
| NDUFB10 | 7.77 | 1.06E−13 | 1.44 | CENPT | 4.53 | 3.83E−05 | 0.42 | CDSN | 3.07 | 9.43E−03 | 0.58 | ZNHIT2 | 2.2 | 9.49E−02 | 1.67 |
| SCAND1 | 7.77 | 1.09E−13 | 1.16 | ANKS3 | 4.53 | 3.82E−05 | 0.36 | MAPK3 | 3.07 | 9.67E−03 | 0.56 | AP3S2 | 2.2 | 9.47E−02 | 0.94 |
| ETV5 | 7.77 | 1.06E−13 | 1.05 | KPNAI | 4.53 | 3.76E−05 | 0.29 | TMEM127 | 3.07 | 9.53E−03 | 0.48 | ZNF134 | 2.2 | 9.62E−02 | 0.93 |
| RPS6 | 7.76 | 1.14E−13 | 0.6 | COX17 | 4.52 | 3.88E−05 | 1.06 | WDR78 | 3.07 | 9.55E−03 | 0.41 | RGS19 | 2.2 | 9.53E−02 | 0.83 |
| SH3PXD2B | 7.76 | 1.14E−13 | 0.57 | GALNTL6 | 4.52 | 3.94E−05 | 1.02 | KLC3 | 3.06 | 9.94E−03 | 1.07 | SNAP25-AS1 | 2.2 | 9.58E−02 | 0.72 |
| GPS2 | 7.75 | 1.23E−13 | 1.3 | UBE2L3 | 4.52 | 4.03E−05 | 0.36 | PPM1E | 3.06 | 9.76E−03 | 1.03 | MAPK11 | 2.2 | 9.64E−02 | 0.64 |
| PIGQ | 7.75 | 1.25E−13 | 0.9 | CEP290 | 4.52 | 3.95E−05 | 0.32 | C19orf18 | 3.06 | 9.92E−03 | 0.92 | MFSD1 | 2.2 | 9.61E−02 | 0.32 |
| FBLN1 | 7.74 | 1.31E−13 | 1.5 | GATA5 | 4.51 | 4.05E−05 | 3.22 | NOMO2 | 3.06 | 9.85E−03 | 0.82 | GJB3 | 2.2 | 9.61E−02 | 0.24 |
| PIEZO1 | 7.73 | 1.43E−13 | 0.59 | EEF1A2 | 4.51 | 4.18E−05 | 1.17 | NDUFA6 | 3.06 | 9.92E−03 | 0.82 | TRIM28 | 2.2 | 9.56E−02 | 0.21 |
| NPRL3 | 7.73 | 1.46E−13 | 0.59 | RFXANK | 4.51 | 4.14E−05 | 0.74 | STMN3 | 3.06 | 9.82E−03 | 0.67 | ATRN | 2.2 | 9.57E−02 | 0.2 |
| RAB11A | 7.71 | 1.64E−13 | 0.43 | PTK6 | 4.51 | 4.15E−05 | 0.31 | TBC1D10B | 3.06 | 9.82E−03 | 0.57 | CIZ1 | 2.2 | 9.64E−02 | 0.2 |
| SKIL | 7.71 | 1.66E−13 | 0.35 | GFER | 4.5 | 4.41E−05 | 1.36 | UBOX5 | 3.06 | 9.84E−03 | 0.53 | SCNNIG | 2.19 | 9.74E−02 | 1.4 |
| NFE2L1 | 7.7 | 1.80E−13 | 0.95 | GOT2 | 4.5 | 4.26E−05 | 0.61 | FBL | 3.06 | 9.73E−03 | 0.48 | MGAT5B | 2.19 | 9.70E−02 | 1.33 |
| FGF19 | 7.67 | 2.24E−13 | 3.55 | NT5E | 4.49 | 4.64E−05 | 0.95 | ADAT1 | 3.06 | 9.81E−03 | 0.47 | CACNA1B | 2.19 | 9.70E−02 | 1.28 |
| NDUFA3 | 7.65 | 2.66E−13 | 2.09 | UQCRFS1 | 4.49 | 4.64E−05 | 0.91 | TMEM50A | 3.06 | 9.84E−03 | 0.29 | PALM2-AKAP2 | 2.19 | 9.72E−02 | 0.88 |
| CEACAM19 | 7.65 | 2.76E−13 | 1.05 | RELB | 4.49 | 4.64E−05 | 0.37 | RXRA | 3.06 | 9.95E−03 | 0.25 | PIK3CD | 2.19 | 9.80E−02 | 0.73 |
| MUC13 | 7.63 | 3.04E−13 | 1.2 | RPL5 | 4.48 | 4.78E−05 | 0.31 | ZFP82 | 3.05 | 1.01E−02 | 1.63 | ZNF699 | 2.19 | 9.87E−02 | 0.71 |
| PRDX1 | 7.63 | 3.16E−13 | 1.01 | IKBIP | 4.47 | 4.97E−05 | 1.04 | C2 | 3.05 | 1.00E−02 | 0.97 | CNPPD1 | 2.19 | 9.82E−02 | 0.63 |
| HMGA1 | 7.63 | 3.05E−13 | 0.86 | SMIM8 | 4.47 | 4.91E−05 | 0.75 | TSEN34 | 3.05 | 1.02E−02 | 0.83 | HTRA2 | 2.19 | 9.77E−02 | 0.62 |
| ASTN2 | 7.63 | 3.01E−13 | 0.83 | TRAPPC6A | 4.47 | 4.88E−05 | 0.65 | ATP2A1 | 3.05 | 1.00E−02 | 0.77 | ADAL | 2.19 | 9.74E−02 | 0.53 |
| PHACTR3 | 7.62 | 3.39E−13 | 0.83 | TAOK2 | 4.47 | 5.01E−05 | 0.63 | CYB561A3 | 3.05 | 1.01E−02 | 0.77 | CCDC142 | 2.19 | 9.70E−02 | 0.46 |
| PSORS1C1 | 7.6 | 4.01E−13 | 0.63 | DHX34 | 4.47 | 4.96E−05 | 0.41 | KLC2 | 3.05 | 1.00E−02 | 0.72 | SUPT4H1 | 2.19 | 9.72E−02 | 0.44 |
| LEMD1 | 7.6 | 4.03E−13 | 0.55 | CCDC124 | 4.46 | 5.13E−05 | 0.81 | GCLM | 3.05 | 1.02E−02 | 0.45 | BBC3 | 2.19 | 9.84E−02 | 0.26 |
| CNTN5 | 7.59 | 4.24E−13 | 1.42 | CSK | 4.46 | 5.14E−05 | 0.77 | ERAP2 | 3.05 | 1.02E−02 | 0.31 | SLC16A4 | 2.19 | 9.87E−02 | 0.24 |
| KRT10 | 7.58 | 4.54E−13 | 0.93 | AMIGO2 | 4.46 | 5.19E−05 | 0.76 | BCL2L14 | 3.05 | 1.00E−02 | 0.24 | POMP | 2.19 | 9.68E−02 | 0.19 |
| SLC51A | 7.57 | 4.74E−13 | 1.81 | CYC1 | 4.46 | 5.20E−05 | 0.62 | ACO2 | 3.05 | 1.02E−02 | 0.2 | CD38 | 2.18 | 9.93E−02 | 1.43 |
| EIF5B | 7.57 | 4.79E−13 | 0.44 | MKKS | 4.45 | 5.36E−05 | 0.81 | ZRANB3 | 3.05 | 1.02E−02 | 0.2 | AC087501.1 | 2.18 | 1.00E−01 | 1.14 |
| CHMP2A | 7.56 | 5.35E−13 | 1.17 | FHAD1 | 4.45 | 5.38E−05 | 0.73 | P2RX7 | 3.04 | 1.04E−02 | 1.65 | IQCD | 2.18 | 1.00E−01 | 1.05 |
| MZT2B | 7.56 | 5.36E−13 | 1 | VDR | 4.45 | 5.50E−05 | 0.42 | POP7 | 3.04 | 1.04E−02 | 1.29 | GLA | 2.18 | 9.88E−02 | 0.87 |
| HOOK2 | 7.56 | 5.36E−13 | 0.43 | ANAPC5 | 4.45 | 5.37E−05 | 0.29 | RHOG | 3.04 | 1.06E−02 | 0.83 | MRPL49 | 2.18 | 9.89E−02 | 0.84 |
| TBCE | 7.55 | 5.61E−13 | 1.78 | PSME2 | 4.44 | 5.63E−05 | 0.91 | POLDIP2 | 3.04 | 1.03E−02 | 0.5 | MN1 | 2.18 | 1.01E−01 | 0.67 |
| CHST3 | 7.55 | 5.56E−13 | 1.36 | CATSPERG | 4.44 | 5.56E−05 | 0.68 | COA4 | 3.04 | 1.05E−02 | 0.5 | IL16 | 2.18 | 9.88E−02 | 0.54 |
| SRPRB | 7.55 | 5.58E−13 | 1.17 | LFNG | 4.44 | 5.63E−05 | 0.58 | PPP1R26-AS1 | 3.04 | 1.03E−02 | 0.39 | NTAN1 | 2.18 | 1.00E−01 | 0.37 |
| HIST2H2BE | 7.55 | 5.56E−13 | 0.86 | CLIP1 | 4.44 | 5.77E−05 | 0.24 | GMPPA | 3.04 | 1.04E−02 | 0.36 | ST3GAL4 | 2.18 | 9.88E−02 | 0.35 |
| PPIA | 7.55 | 5.88E−13 | 0.64 | SLC38A5 | 4.43 | 5.86E−05 | 1.08 | AMDHD2 | 3.03 | 1.08E−02 | 0.46 | SETD1B | 2.18 | 9.92E−02 | 0.32 |
| LOXL4 | 7.54 | 6.03E−13 | 1.61 | SYVN1 | 4.43 | 6.06E−05 | 0.61 | PRKAB1 | 3.03 | 1.08E−02 | 0.34 | PDE4A | 2.18 | 9.99E−02 | 0.29 |
| CSRP1 | 7.54 | 6.24E−13 | 1.19 | BAG6 | 4.43 | 5.88E−05 | 0.47 | ITGAE | 3.02 | 1.11E−02 | 0.52 | E4F1 | 2.18 | 1.01E−01 | 0.26 |
| PDIA3 | 7.54 | 6.27E−13 | 0.65 | HPS5 | 4.43 | 6.00E−05 | 0.39 | H2AFZ | 3.02 | 1.12E−02 | 0.4 | MCRS1 | 2.18 | 9.91E−02 | 0.24 |
| PSMB7 | 7.54 | 5.91E−13 | 0.5 | S100A13 | 4.43 | 5.88E−05 | 0.35 | IL2RG | 3.01 | 1.13E−02 | 3.88 | KCNMB3 | 2.18 | 9.95E−02 | 0.24 |
| GDI1 | 7.53 | 6.40E−13 | 0.72 | CRTC3 | 4.43 | 6.00E−05 | 0.31 | MORN3 | 3.01 | 1.15E−02 | 1.46 | TEC | 2.18 | 1.00E−01 | 0.22 |
| RASSF7 | 7.53 | 6.49E−13 | 0.53 | GNB1L | 4.42 | 6.06E−05 | 0.94 | RSAD2 | 3.01 | 1.15E−02 | 1.05 | WDTC1 | 2.18 | 9.99E−02 | 0.2 |
| NDUFB7 | 7.52 | 7.03E−13 | 1.1 | EHD2 | 4.42 | 6.22E−05 | 0.58 | EFHC2 | 3.01 | 1.13E−02 | 0.84 | DUSP18 | 2.18 | 9.87E−02 | 0.19 |
| MUC16 | 7.5 | 8.41E−13 | 0.8 | MRPL20 | 4.42 | 6.33E−05 | 0.5 | NFKBIL1 | 3.01 | 1.16E−02 | 0.69 | LASP1 | 2.18 | 9.93E−02 | 0.12 |
| HES1 | 7.5 | 8.59E−13 | 0.73 | ATRAID | 4.41 | 6.60E−05 | 0.95 | MLST8 | 3.01 | 1.13E−02 | 0.68 | NPPA | 2.17 | 1.02E−01 | 2.06 |
| YARS | 7.47 | 1.05E−12 | 0.69 | BTBD9 | 4.41 | 6.52E−05 | 0.19 | COQ5 | 3.01 | 1.16E−02 | 0.5 | JAKMIP2 | 2.17 | 1.02E−01 | 1.21 |
| ZNF428 | 7.45 | 1.21E−12 | 1.24 | 1-Sep | 4.4 | 6.73E−05 | 1.72 | MECR | 3.01 | 1.16E−02 | 0.49 | SNHG21 | 2.17 | 1.03E−01 | 0.77 |
| NDUFS8 | 7.45 | 1.19E−12 | 0.97 | ORMDL3 | 4.4 | 6.87E−05 | 1.14 | ATG2A | 3.01 | 1.13E−02 | 0.37 | ANXA6 | 2.17 | 1.01E−01 | 0.34 |
| IFI27L2 | 7.44 | 1.26E−12 | 1.66 | KCTD10 | 4.4 | 6.85E−05 | 0.78 | SZRD1 | 3.01 | 1.14E−02 | 0.36 | FARSA | 2.17 | 1.02E−01 | 0.3 |
| CLDN11 | 7.43 | 1.40E−12 | 3.8 | PSMB3 | 4.4 | 6.91E−05 | 0.57 | IQCH-AS1 | 3.01 | 1.16E−02 | 0.36 | COPS6 | 2.17 | 1.02E−01 | 0.28 |
| STEAP1B | 7.43 | 1.43E−12 | 0.89 | NPIPB4 | 4.4 | 6.86E−05 | 0.52 | PLOD1 | 3.01 | 1.16E−02 | 0.29 | NDUFB1 | 2.17 | 1.03E−01 | 0.25 |
| RNF181 | 7.41 | 1.64E−12 | 2.1 | GDE1 | 4.4 | 6.87E−05 | 0.51 | TMEM219 | 3.01 | 1.14E−02 | 0.28 | EEF2K | 2.17 | 1.02E−01 | 0.16 |
| ARL5A | 7.41 | 1.66E−12 | 0.86 | COASY | 4.4 | 6.67E−05 | 0.48 | PABPC4 | 3.01 | 1.14E−02 | 0.26 | RFNG | 2.17 | 1.02E−01 | 0.15 |
| RPS7 | 7.41 | 1.62E−12 | 0.71 | FBLIM1 | 4.4 | 6.69E−05 | 0.3 | PEX14 | 3.01 | 1.15E−02 | 0.21 | ALOX5 | 2.17 | 1.02E−01 | 0.14 |
| LINC01133 | 7.41 | 1.64E−12 | 0.52 | MVP | 4.4 | 6.64E−05 | 0.27 | PFDN4 | 3 | 1.17E−02 | 0.71 | XRCC6 | 2.17 | 1.03E−01 | 0.14 |
| MMP2 | 7.4 | 1.78E−12 | 1.39 | DHX57 | 4.39 | 6.99E−05 | 0.43 | TPRG1L | 3 | 1.18E−02 | 0.71 | HMG20B | 2.17 | 1.03E−01 | 0.13 |
| IFI35 | 7.4 | 1.77E−12 | 1.37 | PPP1R9B | 4.38 | 7.52E−05 | 1.25 | VWA1 | 3 | 1.17E−02 | 0.59 | AL450992.2 | 2.16 | 1.04E−01 | 2.1 |
| ALKBH6 | 7.4 | 1.72E−12 | 1.24 | PHTF1 | 4.38 | 7.38E−05 | 0.46 | KPNA2 | 3 | 1.18E−02 | 0.44 | GRK7 | 2.16 | 1.04E−01 | 1.78 |
| NDUFS6 | 7.39 | 1.84E−12 | 1.03 | AKT2 | 4.38 | 7.37E−05 | 0.37 | ZFAND2B | 3 | 1.17E−02 | 0.34 | ZNF730 | 2.16 | 1.04E−01 | 1.76 |
| CST3 | 7.38 | 2.06E−12 | 0.86 | LZTR1 | 4.37 | 7.78E−05 | 0.58 | ARL17B | 3 | 1.17E−02 | 0.16 | PI3 | 2.16 | 1.05E−01 | 1.69 |
| DNTTIP1 | 7.38 | 2.00E−12 | 0.76 | IFT74 | 4.37 | 7.77E−05 | 0.47 | GDPGP1 | 2.99 | 1.20E−02 | 2.58 | FUOM | 2.16 | 1.05E−01 | 1.64 |
| CATSPER2 | 7.37 | 2.14E−12 | 0.54 | IFIT2 | 4.36 | 8.20E−05 | 2.18 | PSAT1 | 2.99 | 1.21E−02 | 1.59 | TTLL9 | 2.16 | 1.03E−01 | 1.19 |
| GUK1 | 7.37 | 2.09E−12 | 0.5 | KIAA2013 | 4.36 | 8.22E−05 | 0.96 | SNAI3 | 2.99 | 1.23E−02 | 1.42 | SERTAD4-AS1 | 2.16 | 1.04E−01 | 1.19 |
| ELN | 7.36 | 2.30E−12 | 1.51 | DLGAP1-AS1 | 4.36 | 7.92E−05 | 0.84 | RPUSD1 | 2.99 | 1.22E−02 | 1.17 | PAX6 | 2.16 | 1.03E−01 | 0.95 |
| UQCR10 | 7.35 | 2.47E−12 | 1.18 | SSNA1 | 4.36 | 8.06E−05 | 0.78 | PLBD2 | 2.99 | 1.23E−02 | 0.84 | G6PD | 2.16 | 1.04E−01 | 0.67 |
| CD81 | 7.35 | 2.54E−12 | 1.03 | PARVB | 4.36 | 8.06E−05 | 0.75 | GUCD1 | 2.99 | 1.21E−02 | 0.65 | TLE2 | 2.16 | 1.04E−01 | 0.5 |
| IDS | 7.35 | 2.49E−12 | 0.81 | VWDE | 4.36 | 7.97E−05 | 0.57 | TUBG2 | 2.99 | 1.21E−02 | 0.35 | TWISTNB | 2.16 | 1.04E−01 | 0.45 |
| GDA | 7.35 | 2.54E−12 | 0.49 | LINC00662 | 4.36 | 8.19E−05 | 0.48 | EXOSC1 | 2.99 | 1.22E−02 | 0.29 | MAN2B1 | 2.16 | 1.05E−01 | 0.41 |
| GRAMD1A | 7.33 | 2.93E−12 | 0.52 | CNDP2 | 4.36 | 8.21E−05 | 0.43 | ARL16 | 2.99 | 1.22E−02 | 0.29 | RNF41 | 2.16 | 1.03E−01 | 0.34 |
| ATXN7 | 7.32 | 3.02E−12 | 0.73 | RPS3 | 4.36 | 8.10E−05 | 0.33 | PSMB1 | 2.99 | 1.22E−02 | 0.25 | YIPF5 | 2.16 | 1.04E−01 | 0.31 |
| UBL5 | 7.31 | 3.35E−12 | 1.23 | TMEM132D | 4.35 | 8.57E−05 | 1.07 | JTB | 2.99 | 1.20E−02 | 0.23 | OXA1L | 2.16 | 1.04E−01 | 0.26 |
| SNRPA | 7.31 | 3.43E−12 | 0.69 | TPRA1 | 4.35 | 8.59E−05 | 0.77 | TSTA3 | 2.99 | 1.20E−02 | 0.19 | UROS | 2.16 | 1.04E−01 | 0.25 |
| PPP1R13L | 7.3 | 3.60E−12 | 0.43 | ADAM8 | 4.35 | 8.33E−05 | 0.59 | ZBED3-AS1 | 2.98 | 1.24E−02 | 4.52 | TTLL12 | 2.16 | 1.04E−01 | 0.23 |
| CFAP54 | 7.29 | 3.74E−12 | 1.41 | TSC22D4 | 4.35 | 8.59E−05 | 0.52 | LSM2 | 2.98 | 1.25E−02 | 0.87 | NADSYN1 | 2.16 | 1.04E−01 | 0.18 |
| PRUNE2 | 7.28 | 4.01E−12 | 1.56 | ZNF7 | 4.35 | 8.36E−05 | 0.49 | VPS18 | 2.98 | 1.24E−02 | 0.85 | TTC25 | 2.15 | 1.06E−01 | 0.86 |
| PRR13 | 7.28 | 4.07E−12 | 0.85 | LAMA2 | 4.35 | 8.44E−05 | 0.49 | ABCA2 | 2.98 | 1.26E−02 | 0.56 | CHST6 | 2.15 | 1.06E−01 | 0.86 |
| ZBP1 | 7.27 | 4.37E−12 | 2.46 | SGCD | 4.34 | 8.70E−05 | 1.02 | HS6ST1 | 2.98 | 1.27E−02 | 0.46 | ZNF778 | 2.15 | 1.06E−01 | 0.49 |
| MAGI2 | 7.27 | 4.54E−12 | 0.51 | NAA60 | 4.34 | 8.92E−05 | 0.65 | SIRT6 | 2.98 | 1.24E−02 | 0.24 | HMGN5 | 2.15 | 1.07E−01 | 0.49 |
| CCND2 | 7.26 | 4.76E−12 | 1.19 | SIDT2 | 4.34 | 8.75E−05 | 0.58 | AZIN2 | 2.97 | 1.28E−02 | 0.85 | RBFOX1 | 2.15 | 1.08E−01 | 0.43 |
| CAPZB | 7.26 | 4.81E−12 | 0.36 | RNF40 | 4.34 | 8.69E−05 | 0.46 | CACFD1 | 2.97 | 1.30E−02 | 0.81 | FIS1 | 2.15 | 1.07E−01 | 0.25 |
| AHNAK | 7.26 | 4.73E−12 | 0.31 | PPP1R7 | 4.34 | 8.68E−05 | 0.4 | TTLL1 | 2.97 | 1.28E−02 | 0.65 | ARHGAP31 | 2.15 | 1.06E−01 | 0.24 |
| XDH | 7.25 | 5.30E−12 | 0.6 | CUEDC1 | 4.34 | 8.68E−05 | 0.29 | DDX51 | 2.97 | 1.29E−02 | 0.59 | PTDSS2 | 2.15 | 1.08E−01 | 0.19 |
| PCDH7 | 7.25 | 4.99E−12 | 0.39 | ATP6V1F | 4.33 | 9.05E−05 | 1 | ANKRD52 | 2.97 | 1.30E−02 | 0.53 | HK2 | 2.15 | 1.07E−01 | 0.13 |
| TRMT112 | 7.24 | 5.38E−12 | 1.55 | IMP3 | 4.33 | 9.37E−05 | 0.97 | DEDD2 | 2.97 | 1.31E−02 | 0.51 | STK40 | 2.15 | 1.06E−01 | 0.11 |
| ZNF76 | 7.24 | 5.71E−12 | 0.85 | RRAD | 4.32 | 9.76E−05 | 1.41 | PGRMC1 | 2.97 | 1.29E−02 | 0.49 | NPM3 | 2.14 | 1.08E−01 | 0.71 |
| CEACAM1 | 7.24 | 5.38E−12 | 0.73 | FMN2 | 4.32 | 9.70E−05 | 1.35 | SNRPE | 2.97 | 1.29E−02 | 0.42 | ATP8B3 | 2.14 | 1.08E−01 | 0.63 |
| WBP2 | 7.24 | 5.33E−12 | 0.63 | AURKAIP1 | 4.32 | 9.49E−05 | 0.81 | NSUN5 | 2.97 | 1.28E−02 | 0.31 | TMEM167B | 2.14 | 1.09E−01 | 0.6 |
| GCG | 7.23 | 5.94E−12 | 3.09 | GGACT | 4.32 | 9.62E−05 | 0.69 | ADCY5 | 2.96 | 1.34E−02 | 1.9 | CXCL17 | 2.14 | 1.08E−01 | 0.53 |
| RHOQ | 7.23 | 5.80E−12 | 0.84 | TMEM63B | 4.32 | 9.66E−05 | 0.37 | SURF1 | 2.96 | 1.33E−02 | 0.65 | RNF167 | 2.14 | 1.09E−01 | 0.49 |
| APOL2 | 7.23 | 5.79E−12 | 0.57 | ZSWIM4 | 4.32 | 9.76E−05 | 0.31 | AGFG2 | 2.96 | 1.31E−02 | 0.44 | JRK | 2.14 | 1.10E−01 | 0.24 |
| PAK4 | 7.22 | 6.53E−12 | 0.6 | IFITM10 | 4.31 | 1.02E−04 | 1.8 | ZRSR2 | 2.96 | 1.32E−02 | 0.43 | SDF2 | 2.14 | 1.08E−01 | 0.2 |
| VCP | 7.2 | 7.31E−12 | 0.52 | LOXL1-AS1 | 4.31 | 9.94E−05 | 1.35 | ZFAND5 | 2.96 | 1.34E−02 | 0.23 | SDHB | 2.14 | 1.09E−01 | 0.15 |
| SEC61G | 7.19 | 8.02E−12 | 1.34 | TRIB3 | 4.31 | 1.01E−04 | 1.29 | ECHDC2 | 2.96 | 1.32E−02 | 0.17 | CEP126 | 2.14 | 1.09E−01 | 0.13 |
| BCAM | 7.19 | 7.95E−12 | 1.26 | SWI5 | 4.31 | 9.93E−05 | 1 | CLIP2 | 2.96 | 1.34E−02 | 0.17 | RASGRF1 | 2.13 | 1.11E−01 | 1.91 |
| SLC7A6 | 7.19 | 8.08E−12 | 0.8 | MRPL14 | 4.31 | 9.87E−05 | 0.73 | GBP1 | 2.95 | 1.35E−02 | 0.71 | FNDC4 | 2.13 | 1.10E−01 | 0.95 |
| TXN | 7.18 | 8.18E−12 | 0.71 | PLXND1 | 4.31 | 1.02E−04 | 0.73 | NEXN | 2.95 | 1.38E−02 | 0.59 | THEM6 | 2.13 | 1.11E−01 | 0.78 |
| ODF2 | 7.16 | 9.92E−12 | 0.76 | ISG20 | 4.31 | 1.00E−04 | 0.51 | DDOST | 2.95 | 1.35E−02 | 0.53 | FBXO17 | 2.13 | 1.11E−01 | 0.43 |
| COX4I1 | 7.16 | 9.54E−12 | 0.65 | COPZ1 | 4.31 | 9.86E−05 | 0.47 | PIK3IP1-AS1 | 2.95 | 1.37E−02 | 0.49 | GPC4 | 2.13 | 1.11E−01 | 0.29 |
| ALDH1A1 | 7.15 | 1.06E−11 | 1.06 | MRPL36 | 4.3 | 1.03E−04 | 1.73 | LINC00649 | 2.95 | 1.38E−02 | 0.44 | CEBPZOS | 2.13 | 1.11E−01 | 0.18 |
| COL6A3 | 7.15 | 1.05E−11 | 0.65 | FAAP20 | 4.3 | 1.06E−04 | 0.54 | RBM19 | 2.95 | 1.36E−02 | 0.36 | C12orf49 | 2.13 | 1.12E−01 | 0.17 |
| LINC01605 | 7.14 | 1.11E−11 | 1.92 | DHRS3 | 4.3 | 1.06E−04 | 0.28 | SORT1 | 2.95 | 1.36E−02 | 0.35 | TIMM23 | 2.13 | 1.11E−01 | 0.16 |
| MAFG | 7.14 | 1.10E−11 | 1.15 | GOLIM4 | 4.3 | 1.06E−04 | 0.27 | MRPL42 | 2.95 | 1.35E−02 | 0.34 | SRSF3 | 2.13 | 1.11E−01 | 0.14 |
| DYNLL2 | 7.14 | 1.13E−11 | 1.13 | JUP | 4.3 | 1.06E−04 | 0.24 | FZR1 | 2.95 | 1.37E−02 | 0.24 | UBXN2A | 2.13 | 1.12E−01 | 0.14 |
| CDKN1A | 7.13 | 1.24E−11 | 0.85 | NFIA | 4.3 | 1.04E−04 | 0.18 | TRPV4 | 2.95 | 1.38E−02 | 0.24 | ZNF444 | 2.13 | 1.10E−01 | 0.13 |
| SLC8B1 | 7.13 | 1.23E−11 | 0.84 | PYCR1 | 4.29 | 1.09E−04 | 1.12 | COQ4 | 2.95 | 1.36E−02 | 0.23 | RNF220 | 2.13 | 1.10E−01 | 0.11 |
| CACNA2D1 | 7.13 | 1.21E−11 | 0.81 | KIF7 | 4.29 | 1.07E−04 | 1 | VPS35 | 2.95 | 1.38E−02 | 0.22 | ITFG1 | 2.13 | 1.12E−01 | 0.06 |
| TCEAL4 | 7.12 | 1.28E−11 | 0.96 | RNF187 | 4.29 | 1.08E−04 | 0.77 | PCMT1 | 2.95 | 1.37E−02 | 0.21 | MIR181A1HG | 2.13 | 1.12E−01 | 0.04 |
| KNOP1 | 7.12 | 1.32E−11 | 0.94 | PDP2 | 4.29 | 1.08E−04 | 0.54 | RING1 | 2.95 | 1.36E−02 | 0.18 | HOXB-AS1 | 2.12 | 1.13E−01 | 1.11 |
| KIFC3 | 7.12 | 1.28E−11 | 0.53 | PLPP4 | 4.28 | 1.13E−04 | 1.13 | C20orf194 | 2.95 | 1.36E−02 | 0.18 | MAP4K2 | 2.12 | 1.14E−01 | 0.64 |
| FUT2 | 7.11 | 1.38E−11 | 0.97 | ATG101 | 4.28 | 1.13E−04 | 0.89 | C4orf48 | 2.94 | 1.39E−02 | 0.53 | SLC5A6 | 2.12 | 1.14E−01 | 0.52 |
| ELMOD3 | 7.11 | 1.36E−11 | 0.59 | ARID3A | 4.27 | 1.19E−04 | 0.48 | B4GALT2 | 2.94 | 1.40E−02 | 0.42 | FAHD1 | 2.12 | 1.13E−01 | 0.49 |
| WNT2B | 7.1 | 1.47E−11 | 0.92 | PTPRD | 4.27 | 1.17E−04 | 0.44 | EIF4H | 2.94 | 1.39E−02 | 0.25 | SLC35E2B | 2.12 | 1.14E−01 | 0.28 |
| IQCJ-SCHIP1 | 7.1 | 1.48E−11 | 0.69 | PTCHD1 | 4.26 | 1.23E−04 | 1.87 | SFSWAP | 2.94 | 1.41E−02 | 0.24 | NR2F2 | 2.12 | 1.15E−01 | 0.28 |
| NDUFS5 | 7.08 | 1.76E−11 | 1.04 | PRNP | 4.26 | 1.26E−04 | 0.93 | ST3GAL2 | 2.94 | 1.42E−02 | 0.24 | GTF3A | 2.12 | 1.13E−01 | 0.25 |
| TMED3 | 7.08 | 1.72E−11 | 0.75 | SAA1 | 4.26 | 1.25E−04 | 0.9 | D2HGDH | 2.94 | 1.40E−02 | 0.23 | SPTY2D1 | 2.12 | 1.13E−01 | 0.24 |
| IFIT1 | 7.07 | 1.81E−11 | 2.81 | MRPS10 | 4.26 | 1.22E−04 | 0.76 | OSBP | 2.94 | 1.40E−02 | 0.22 | LINC00894 | 2.12 | 1.14E−01 | 0.23 |
| TERF2IP | 7.07 | 1.86E−11 | 0.55 | RPS21 | 4.26 | 1.26E−04 | 0.47 | CCDC14 | 2.94 | 1.40E−02 | 0.19 | MRPL4 | 2.12 | 1.14E−01 | 0.21 |
| AAK1 | 7.07 | 1.82E−11 | 0.35 | SNX19 | 4.26 | 1.26E−04 | 0.41 | IER3 | 2.94 | 1.39E−02 | 0.18 | UNC93B1 | 2.12 | 1.13E−01 | 0.2 |
| MSRB3 | 7.06 | 2.04E−11 | 1.26 | PIPOX | 4.25 | 1.29E−04 | 1.18 | HSPA14 | 2.93 | 1.45E−02 | 2.72 | COX7B | 2.12 | 1.15E−01 | 0.19 |
| SERINC2 | 7.06 | 1.99E−11 | 0.57 | PEX10 | 4.25 | 1.31E−04 | 0.8 | SLC2A12 | 2.93 | 1.45E−02 | 1.2 | ZMIZ2 | 2.12 | 1.13E−01 | 0.17 |
| NTF4 | 7.05 | 2.13E−11 | 4.06 | MMAB | 4.25 | 1.27E−04 | 0.71 | EMC4 | 2.93 | 1.46E−02 | 0.8 | DDX24 | 2.12 | 1.13E−01 | 0.17 |
| SLC1A5 | 7.05 | 2.06E−11 | 1.39 | DZIP3 | 4.25 | 1.30E−04 | 0.45 | RPL18A | 2.93 | 1.44E−02 | 0.76 | SNHG12 | 2.12 | 1.14E−01 | 0.17 |
| EMC10 | 7.04 | 2.26E−11 | 0.85 | PSMD7 | 4.25 | 1.30E−04 | 0.35 | KSR2 | 2.93 | 1.45E−02 | 0.68 | SOX9 | 2.12 | 1.15E−01 | 0.17 |
| ANAPC2 | 7.03 | 2.46E−11 | 1.33 | PSMD11 | 4.25 | 1.31E−04 | 0.34 | KLRD1 | 2.93 | 1.43E−02 | 0.67 | TMEM109 | 2.11 | 1.16E−01 | 0.87 |
| C16orf46 | 7.02 | 2.58E−11 | 4.01 | BNC2 | 4.25 | 1.31E−04 | 0.33 | NR1D1 | 2.93 | 1.44E−02 | 0.57 | STK31 | 2.11 | 1.16E−01 | 0.58 |
| IL34 | 7.02 | 2.52E−11 | 2.14 | USP36 | 4.25 | 1.27E−04 | 0.31 | LIPA | 2.93 | 1.46E−02 | 0.48 | SPRY2 | 2.11 | 1.16E−01 | 0.35 |
| LYPD6B | 7.02 | 2.56E−11 | 0.53 | SHANK2 | 4.25 | 1.30E−04 | 0.19 | PSME3 | 2.93 | 1.43E−02 | 0.47 | SYF2 | 2.11 | 1.15E−01 | 0.34 |
| GOLGA3 | 7.02 | 2.65E−11 | 0.51 | EFEMP1 | 4.24 | 1.34E−04 | 1.08 | ERGIC3 | 2.93 | 1.44E−02 | 0.24 | IAH1 | 2.11 | 1.16E−01 | 0.32 |
| IQCK | 7.01 | 2.84E−11 | 0.49 | ERLEC1 | 4.24 | 1.36E−04 | 0.6 | GAN | 2.93 | 1.44E−02 | 0.19 | TXNDC9 | 2.11 | 1.16E−01 | 0.24 |
| FADS3 | 7 | 3.07E−11 | 1.4 | LMAN2 | 4.24 | 1.32E−04 | 0.52 | HERC5 | 2.92 | 1.50E−02 | 1.28 | LMO4 | 2.11 | 1.15E−01 | 0.21 |
| OFD1 | 7 | 2.99E−11 | 0.42 | SARS | 4.24 | 1.34E−04 | 0.43 | MIF4GD | 2.92 | 1.48E−02 | 1.16 | LINC01524 | 2.11 | 1.16E−01 | 0.19 |
| DNAJA3 | 6.98 | 3.40E−11 | 0.66 | NFKBIZ | 4.24 | 1.33E−04 | 0.28 | MTUS2 | 2.92 | 1.51E−02 | 1.08 | CPSF1 | 2.11 | 1.16E−01 | 0.18 |
| NUCB1 | 6.97 | 3.72E−11 | 0.78 | ZNF439 | 4.23 | 1.40E−04 | 0.85 | MRPL11 | 2.92 | 1.48E−02 | 0.78 | PHB2 | 2.11 | 1.15E−01 | 0.12 |
| RBMS3 | 6.97 | 3.77E−11 | 0.58 | TIMM13 | 4.23 | 1.39E−04 | 0.63 | ZMAT2 | 2.92 | 1.47E−02 | 0.46 | KCNEl | 2.1 | 1.18E−01 | 1.73 |
| DST | 6.97 | 3.62E−11 | 0.18 | PGAM1 | 4.23 | 1.40E−04 | 0.46 | ADPRHL2 | 2.92 | 1.48E−02 | 0.41 | EFCAB8 | 2.1 | 1.18E−01 | 1.47 |
| DNAJB2 | 6.96 | 4.07E−11 | 0.81 | NACA2 | 4.22 | 1.47E−04 | 1.26 | FAM136A | 2.92 | 1.47E−02 | 0.39 | PPIC | 2.1 | 1.19E−01 | 0.45 |
| AHDC1 | 6.96 | 4.05E−11 | 0.6 | PSMC2 | 4.22 | 1.45E−04 | 0.47 | PPP1R14B | 2.92 | 1.50E−02 | 0.35 | ASB16-AS1 | 2.1 | 1.18E−01 | 0.42 |
| HSF1 | 6.95 | 4.10E−11 | 0.47 | SPDYE3 | 4.22 | 1.49E−04 | 0.45 | ZFP14 | 2.92 | 1.48E−02 | 0.3 | CRACR2B | 2.1 | 1.19E−01 | 0.3 |
| PER1 | 6.94 | 4.59E−11 | 0.57 | RNH1 | 4.22 | 1.46E−04 | 0.35 | ANO1 | 2.92 | 1.47E−02 | 0.22 | GHITM | 2.1 | 1.18E−01 | 0.28 |
| MOB3B | 6.94 | 4.55E−11 | 0.53 | EMC6 | 4.21 | 1.52E−04 | 1.6 | DTNB | 2.92 | 1.48E−02 | 0.16 | DEF6 | 2.1 | 1.18E−01 | 0.27 |
| RPL39 | 6.93 | 4.74E−11 | 1.68 | SERTAD3 | 4.21 | 1.53E−04 | 1.23 | EXOSC4 | 2.91 | 1.51E−02 | 1.17 | MTCH2 | 2.1 | 1.19E−01 | 0.17 |
| TNIP1 | 6.92 | 5.18E−11 | 0.46 | PROSER3 | 4.21 | 1.51E−04 | 0.52 | LRRC75B | 2.91 | 1.52E−02 | 0.67 | RABEP2 | 2.1 | 1.18E−01 | 0.16 |
| UQCRH | 6.88 | 6.90E−11 | 0.81 | ARMCX3 | 4.21 | 1.53E−04 | 0.49 | COX5A | 2.91 | 1.54E−02 | 0.47 | RNF10 | 2.1 | 1.18E−01 | 0.15 |
| DRAP1 | 6.87 | 7.07E−11 | 1.21 | HNRNPA1 | 4.21 | 1.50E−04 | 0.31 | STPG1 | 2.91 | 1.52E−02 | 0.46 | RNF114 | 2.1 | 1.19E−01 | 0.15 |
| TMA7 | 6.87 | 7.41E−11 | 0.85 | ACVR1B | 4.21 | 1.50E−04 | 0.28 | C8orf37-AS1 | 2.91 | 1.55E−02 | 0.41 | PSMG2 | 2.1 | 1.18E−01 | 0.14 |
| ZNF429 | 6.87 | 7.53E−11 | 0.81 | APOO | 4.21 | 1.51E−04 | 0.26 | LINC00843 | 2.91 | 1.52E−02 | 0.34 | BZW1 | 2.1 | 1.19E−01 | 0.09 |
| ZNF75A | 6.87 | 7.28E−11 | 0.76 | SLC39A7 | 4.2 | 1.60E−04 | 0.85 | TMEM92 | 2.9 | 1.60E−02 | 0.87 | IGF2BP1 | 2.09 | 1.20E−01 | 1.58 |
| CBWD5 | 6.87 | 7.21E−11 | 0.48 | SLC9A3R2 | 4.2 | 1.57E−04 | 0.52 | ILDR1 | 2.9 | 1.60E−02 | 0.67 | CLCN4 | 2.09 | 1.23E−01 | 0.73 |
| CYTH3 | 6.86 | 7.90E−11 | 0.6 | RYR3 | 4.19 | 1.63E−04 | 1.27 | ABCD1 | 2.9 | 1.57E−02 | 0.66 | SNX33 | 2.09 | 1.20E−01 | 0.32 |
| HSH2D | 6.85 | 8.46E−11 | 0.71 | GABARAPL2 | 4.19 | 1.68E−04 | 0.75 | TRMT10C | 2.9 | 1.56E−02 | 0.62 | PRR12 | 2.09 | 1.21E−01 | 0.25 |
| NBPF14 | 6.85 | 8.18E−11 | 0.69 | PSME1 | 4.19 | 1.65E−04 | 0.68 | LSM3 | 2.9 | 1.58E−02 | 0.56 | NSMF | 2.09 | 1.22E−01 | 0.23 |
| CAMK2G | 6.85 | 8.24E−11 | 0.45 | GOLT1B | 4.19 | 1.65E−04 | 0.42 | KCNN4 | 2.9 | 1.58E−02 | 0.2 | ARHGEF10L | 2.09 | 1.21E−01 | 0.18 |
| ATG9A | 6.84 | 9.05E−11 | 2.62 | PTPN1 | 4.19 | 1.63E−04 | 0.3 | IRAK3 | 2.9 | 1.57E−02 | 0.18 | CNBP | 2.09 | 1.22E−01 | 0.14 |
| ZBTB16 | 6.84 | 8.97E−11 | 1.03 | CRABP2 | 4.18 | 1.76E−04 | 1.54 | ERC1 | 2.9 | 1.58E−02 | 0.13 | PRDX6 | 2.09 | 1.22E−01 | 0.14 |
| WDR45 | 6.84 | 8.64E−11 | 0.66 | SLIT3 | 4.18 | 1.76E−04 | 1.26 | OR2L13 | 2.89 | 1.65E−02 | 2.71 | TINAGL1 | 2.09 | 1.21E−01 | 0.13 |
| LINC00886 | 6.84 | 8.81E−11 | 0.59 | TIMM50 | 4.18 | 1.71E−04 | 0.81 | CLPSL1 | 2.89 | 1.63E−02 | 2.5 | CLTC | 2.09 | 1.21E−01 | 0.13 |
| P3H2 | 6.84 | 8.69E−11 | 0.34 | CYP27C1 | 4.18 | 1.73E−04 | 0.76 | NRSN2 | 2.89 | 1.62E−02 | 1.69 | HNRNPUL1 | 2.09 | 1.22E−01 | 0.13 |
| GRINA | 6.83 | 9.21E−11 | 0.86 | BACH2 | 4.18 | 1.72E−04 | 0.48 | SCUBE3 | 2.89 | 1.65E−02 | 1.41 | KLK8 | 2.08 | 1.23E−01 | 3.99 |
| DYNLT1 | 6.83 | 9.45E−11 | 0.74 | CALCOC02 | 4.18 | 1.74E−04 | 0.27 | DEGS1 | 2.89 | 1.63E−02 | 0.66 | AC091132.1 | 2.08 | 1.25E−01 | 2.07 |
| SLC14A1 | 6.82 | 9.91E−11 | 2.74 | ARF1 | 4.17 | 1.78E−04 | 0.29 | NIPA2 | 2.89 | 1.62E−02 | 0.21 | SSC5D | 2.08 | 1.25E−01 | 1.19 |
| PALLD | 6.82 | 1.03E−10 | 0.36 | CACNA1C | 4.17 | 1.79E−04 | 0.28 | CDC37 | 2.89 | 1.62E−02 | 0.17 | MSRB1 | 2.08 | 1.25E−01 | 0.8 |
| PFN1 | 6.8 | 1.21E−10 | 0.74 | GLYR1 | 4.17 | 1.78E−04 | 0.26 | BOC | 2.88 | 1.68E−02 | 1.39 | C3orf67-AS1 | 2.08 | 1.24E−01 | 0.64 |
| ARPC3 | 6.8 | 1.14E−10 | 0.47 | U2AF1 | 4.16 | 1.90E−04 | 1.24 | C8orf33 | 2.88 | 1.66E−02 | 0.88 | MRPL21 | 2.08 | 1.24E−01 | 0.63 |
| GGT1 | 6.79 | 1.26E−10 | 1.78 | UFL1-AS1 | 4.16 | 1.92E−04 | 0.64 | MMP15 | 2.88 | 1.67E−02 | 0.7 | DPAGT1 | 2.08 | 1.25E−01 | 0.61 |
| LSM4 | 6.77 | 1.47E−10 | 0.93 | GNG5 | 4.16 | 1.87E−04 | 0.42 | SCPEP1 | 2.88 | 1.67E−02 | 0.41 | ERO1B | 2.08 | 1.24E−01 | 0.43 |
| DNAJC7 | 6.77 | 1.41E−10 | 0.43 | ING4 | 4.16 | 1.89E−04 | 0.37 | SPECC1 | 2.88 | 1.66E−02 | 0.36 | RUNDC1 | 2.08 | 1.25E−01 | 0.26 |
| TANC2 | 6.75 | 1.61E−10 | 0.25 | PLPP3 | 4.15 | 2.00E−04 | 0.81 | IFT27 | 2.88 | 1.66E−02 | 0.29 | PBX2 | 2.08 | 1.24E−01 | 0.2 |
| PTCHD1-AS | 6.74 | 1.73E−10 | 1.21 | CACNB4 | 4.15 | 1.98E−04 | 0.75 | CASP8 | 2.88 | 1.65E−02 | 0.23 | TRIM31-AS1 | 2.08 | 1.25E−01 | 0.19 |
| LSM5 | 6.74 | 1.78E−10 | 0.9 | CRISPLD2 | 4.15 | 1.95E−04 | 0.51 | C6orf106 | 2.88 | 1.65E−02 | 0.15 | SHISA5 | 2.08 | 1.25E−01 | 0.15 |
| FLYWCH1 | 6.74 | 1.72E−10 | 0.63 | NFATC3 | 4.15 | 2.00E−04 | 0.26 | TNRC6B | 2.88 | 1.68E−02 | 0.08 | WIPI2 | 2.08 | 1.23E−01 | 0.14 |
| LDHA | 6.74 | 1.74E−10 | 0.52 | ST6GALNAC6 | 4.14 | 2.09E−04 | 3.99 | PTRHD1 | 2.87 | 1.72E−02 | 1.76 | THUMPD3-AS1 | 2.08 | 1.24E−01 | 0.13 |
| ANKRD27 | 6.73 | 1.87E−10 | 0.51 | PTPRT | 4.14 | 2.03E−04 | 1.15 | SOX12 | 2.87 | 1.71E−02 | 1.2 | MARCKS | 2.08 | 1.23E−01 | 0.12 |
| NMD3 | 6.73 | 1.81E−10 | 0.46 | PFDN6 | 4.14 | 2.05E−04 | 0.97 | MRPS18A | 2.87 | 1.72E−02 | 0.72 | BCL2L13 | 2.08 | 1.24E−01 | 0.12 |
| EML4 | 6.73 | 1.90E−10 | 0.32 | PRKCSH | 4.14 | 2.06E−04 | 0.41 | IMP4 | 2.87 | 1.74E−02 | 0.43 | DPP9 | 2.08 | 1.23E−01 | 0.1 |
| UQCRB | 6.72 | 1.98E−10 | 0.81 | GCN1 | 4.14 | 2.06E−04 | 0.37 | SCGB2B2 | 2.87 | 1.71E−02 | 0.23 | ANXA4 | 2.08 | 1.25E−01 | 0.06 |
| HAGH | 6.72 | 1.97E−10 | 0.73 | DDX18 | 4.14 | 2.01E−04 | 0.34 | ELK3 | 2.87 | 1.75E−02 | 0.22 | ANXA2 | 2.08 | 1.24E−01 | −0.01 |
| COX7A2L | 6.71 | 2.12E−10 | 0.77 | RNF126 | 4.14 | 2.06E−04 | 0.34 | SEMA3B | 2.87 | 1.74E−02 | 0.19 | ZNF486 | 2.07 | 1.27E−01 | 0.67 |
| PLSCR1 | 6.71 | 2.19E−10 | 0.44 | SCAF1 | 4.13 | 2.12E−04 | 0.73 | NAALADL2 | 2.87 | 1.72E−02 | 0.09 | PXDN | 2.07 | 1.26E−01 | 0.65 |
| KIF12 | 6.7 | 2.30E−10 | 1.33 | SLC35B1 | 4.13 | 2.09E−04 | 0.71 | PLEKHF1 | 2.86 | 1.79E−02 | 1.96 | CYB561D1 | 2.07 | 1.26E−01 | 0.47 |
| ERP29 | 6.7 | 2.28E−10 | 0.98 | C4orf47 | 4.13 | 2.15E−04 | 0.66 | OGFOD2 | 2.86 | 1.76E−02 | 0.92 | PDGFB | 2.07 | 1.26E−01 | 0.47 |
| SNRPG | 6.69 | 2.44E−10 | 0.92 | CEBPB | 4.13 | 2.09E−04 | 0.37 | NARS | 2.86 | 1.75E−02 | 0.41 | DDX41 | 2.07 | 1.28E−01 | 0.43 |
| THBS2 | 6.69 | 2.46E−10 | 0.82 | MT-CYB | 4.13 | 2.17E−04 | 0.34 | NR2F6 | 2.86 | 1.76E−02 | 0.24 | LINC00672 | 2.07 | 1.26E−01 | 0.4 |
| RPS29 | 6.68 | 2.66E−10 | 0.67 | PPARD | 4.13 | 2.16E−04 | 0.31 | SAP30BP | 2.86 | 1.77E−02 | 0.19 | GTF3C4 | 2.07 | 1.28E−01 | 0.37 |
| LEKR1 | 6.67 | 2.68E−10 | 0.99 | KLF6 | 4.13 | 2.13E−04 | 0.12 | HECTD4 | 2.86 | 1.75E−02 | 0.17 | ZNF785 | 2.07 | 1.27E−01 | 0.32 |
| PTGES2 | 6.67 | 2.79E−10 | 0.61 | TSSC4 | 4.12 | 2.20E−04 | 0.87 | SLC14A2 | 2.85 | 1.83E−02 | 1.89 | PANX1 | 2.07 | 1.26E−01 | 0.3 |
| TRAM1 | 6.66 | 2.98E−10 | 0.51 | MANSC1 | 4.12 | 2.22E−04 | 0.82 | CXCL16 | 2.85 | 1.83E−02 | 0.96 | ILVBL | 2.07 | 1.26E−01 | 0.27 |
| NELFE | 6.65 | 3.23E−10 | 1.18 | CES2 | 4.12 | 2.24E−04 | 0.5 | ARMC6 | 2.85 | 1.84E−02 | 0.85 | KLHL13 | 2.07 | 1.27E−01 | 0.27 |
| LY6K | 6.64 | 3.43E−10 | 3.08 | ASCC1 | 4.12 | 2.24E−04 | 0.27 | RAB11FIP5 | 2.85 | 1.80E−02 | 0.62 | NSMCE1 | 2.07 | 1.27E−01 | 0.27 |
| COG1 | 6.64 | 3.32E−10 | 0.83 | LINC01609 | 4.11 | 2.27E−04 | 2.08 | HSD11B2 | 2.85 | 1.81E−02 | 0.5 | CHN1 | 2.07 | 1.27E−01 | 0.23 |
| TMEM208 | 6.63 | 3.64E−10 | 1.11 | NXNL2 | 4.11 | 2.31E−04 | 1.45 | ECHS1 | 2.85 | 1.83E−02 | 0.41 | SPG21 | 2.07 | 1.26E−01 | 0.19 |
| ENG | 6.63 | 3.56E−10 | 1.07 | HMGCL | 4.11 | 2.33E−04 | 0.68 | FAM219B | 2.85 | 1.83E−02 | 0.33 | SMC5-AS1 | 2.07 | 1.27E−01 | 0.15 |
| BLCAP | 6.63 | 3.60E−10 | 0.44 | ZNF43 | 4.11 | 2.35E−04 | 0.6 | FAM83H | 2.85 | 1.81E−02 | 0.32 | PLET1 | 2.06 | 1.30E−01 | 2.02 |
| NIPAL3 | 6.62 | 3.87E−10 | 0.66 | PHB | 4.11 | 2.28E−04 | 0.57 | WDR34 | 2.85 | 1.82E−02 | 0.29 | PCSK2 | 2.06 | 1.29E−01 | 1.7 |
| NR2F2-AS1 | 6.61 | 3.98E−10 | 0.56 | GLO1 | 4.11 | 2.31E−04 | 0.57 | GGA2 | 2.85 | 1.83E−02 | 0.18 | RNF5 | 2.06 | 1.30E−01 | 1.05 |
| DYNLL1 | 6.6 | 4.29E−10 | 0.77 | DGCR2 | 4.11 | 2.31E−04 | 0.31 | RASA4B | 2.84 | 1.88E−02 | 3.6 | CXorf40B | 2.06 | 1.29E−01 | 1.02 |
| AC008074.3 | 6.59 | 4.61E−10 | 4 | DLG5 | 4.11 | 2.31E−04 | 0.29 | ZNF385C | 2.84 | 1.85E−02 | 1.45 | PALM | 2.06 | 1.29E−01 | 0.99 |
| RASSF5 | 6.59 | 4.67E−10 | 0.89 | CES4A | 4.1 | 2.40E−04 | 1.32 | LINC01023 | 2.84 | 1.88E−02 | 0.97 | IPO13 | 2.06 | 1.29E−01 | 0.79 |
| MRPL55 | 6.59 | 4.68E−10 | 0.88 | RAB34 | 4.1 | 2.38E−04 | 0.82 | ACADS | 2.84 | 1.87E−02 | 0.82 | AOC1 | 2.06 | 1.29E−01 | 0.7 |
| VASP | 6.58 | 4.89E−10 | 0.38 | UBE2M | 4.1 | 2.44E−04 | 0.81 | TXNDC17 | 2.84 | 1.88E−02 | 0.62 | EIF3C | 2.06 | 1.30E−01 | 0.61 |
| TCEA2 | 6.57 | 5.34E−10 | 1.14 | PHC2 | 4.1 | 2.45E−04 | 0.35 | PITPNM2 | 2.84 | 1.87E−02 | 0.42 | TMEM223 | 2.06 | 1.29E−01 | 0.54 |
| NRXN3 | 6.56 | 5.78E−10 | 1.06 | MGRN1 | 4.1 | 2.38E−04 | 0.32 | SNUPN | 2.84 | 1.90E−02 | 0.42 | EGF | 2.06 | 1.30E−01 | 0.43 |
| AP2S1 | 6.56 | 5.55E−10 | 0.98 | LAMB1 | 4.1 | 2.39E−04 | 0.29 | TMED4 | 2.84 | 1.90E−02 | 0.34 | EFCAB5 | 2.06 | 1.30E−01 | 0.4 |
| ATOX1 | 6.56 | 5.66E−10 | 0.83 | PPIL2 | 4.1 | 2.37E−04 | 0.28 | POLR1A | 2.84 | 1.89E−02 | 0.33 | ERCC2 | 2.06 | 1.28E−01 | 0.34 |
| TSPAN5 | 6.56 | 5.71E−10 | 0.42 | CXCL14 | 4.09 | 2.53E−04 | 1.05 | VRK3 | 2.84 | 1.85E−02 | 0.29 | MRPL2 | 2.06 | 1.29E−01 | 0.34 |
| ABHD11 | 6.55 | 5.88E−10 | 0.65 | C6orf48 | 4.09 | 2.56E−04 | 0.67 | EYA2 | 2.84 | 1.86E−02 | 0.07 | CCDC59 | 2.06 | 1.30E−01 | 0.28 |
| CBX6 | 6.54 | 6.32E−10 | 1.32 | CNTNAP3 | 4.09 | 2.49E−04 | 0.58 | DNAAF3 | 2.83 | 1.94E−02 | 1.64 | RSL1D1 | 2.06 | 1.29E−01 | 0.25 |
| ADRM1 | 6.54 | 6.55E−10 | 1.06 | AAMP | 4.09 | 2.49E−04 | 0.38 | RPP25 | 2.83 | 1.92E−02 | 1.56 | MOCOS | 2.06 | 1.28E−01 | 0.22 |
| MCFD2 | 6.54 | 6.48E−10 | 0.69 | MAVS | 4.09 | 2.46E−04 | 0.32 | IRF2BPL | 2.83 | 1.91E−02 | 0.87 | ARSJ | 2.06 | 1.29E−01 | 0.22 |
| SLC35D2 | 6.54 | 6.57E−10 | 0.52 | ZFPM2 | 4.09 | 2.49E−04 | 0.26 | GATC | 2.83 | 1.94E−02 | 0.78 | GST01 | 2.06 | 1.29E−01 | 0.22 |
| PAFAH1B2 | 6.52 | 7.16E−10 | 0.5 | DOC2A | 4.08 | 2.62E−04 | 1.43 | ZNF540 | 2.83 | 1.91E−02 | 0.7 | FKBP14 | 2.06 | 1.29E−01 | 0.21 |
| HSBP1 | 6.51 | 7.65E−10 | 0.89 | DDIT3 | 4.08 | 2.61E−04 | 0.63 | SEMA3E | 2.83 | 1.93E−02 | 0.66 | HIST1H2BD | 2.06 | 1.29E−01 | 0.21 |
| IARS | 6.51 | 7.86E−10 | 0.45 | FRMD4A | 4.08 | 2.59E−04 | 0.38 | LRPAP1 | 2.83 | 1.93E−02 | 0.51 | PDAP1 | 2.06 | 1.28E−01 | 0.2 |
| COMP | 6.5 | 8.32E−10 | 2.08 | ERGIC2 | 4.08 | 2.57E−04 | 0.29 | LACTB2 | 2.83 | 1.96E−02 | 0.33 | MALT1 | 2.06 | 1.30E−01 | 0.2 |
| PAX8 | 6.5 | 8.37E−10 | 0.6 | MPPED2 | 4.07 | 2.77E−04 | 2.14 | CFAP100 | 2.82 | 2.00E−02 | 2.18 | OSBPL2 | 2.06 | 1.29E−01 | 0.13 |
| PHLDA2 | 6.49 | 8.88E−10 | 0.65 | STARD9 | 4.07 | 2.70E−04 | 1.05 | PLXNA4 | 2.82 | 2.00E−02 | 1.3 | TNFRSF10C | 2.05 | 1.32E−01 | 1.74 |
| GARS | 6.48 | 9.80E−10 | 0.62 | MYO16 | 4.07 | 2.67E−04 | 1.04 | JMJD6 | 2.82 | 2.00E−02 | 0.38 | C1orf216 | 2.05 | 1.32E−01 | 0.98 |
| CD151 | 6.48 | 9.69E−10 | 0.43 | ZNF865 | 4.07 | 2.77E−04 | 0.84 | OGFOD3 | 2.82 | 1.98E−02 | 0.37 | WDR88 | 2.05 | 1.31E−01 | 0.87 |
| PTMS | 6.47 | 1.04E−09 | 1.03 | ENDOD1 | 4.07 | 2.69E−04 | 0.72 | COIL | 2.82 | 1.96E−02 | 0.35 | MIR3681HG | 2.05 | 1.33E−01 | 0.82 |
| ARHGAP22 | 6.47 | 1.03E−09 | 0.81 | SRPRA | 4.07 | 2.74E−04 | 0.68 | CAV1 | 2.82 | 1.98E−02 | 0.28 | KIAA1324 | 2.05 | 1.32E−01 | 0.73 |
| SCO2 | 6.45 | 1.18E−09 | 3.66 | MRFAP1 | 4.07 | 2.77E−04 | 0.48 | ZNF248 | 2.82 | 1.97E−02 | 0.22 | NME5 | 2.05 | 1.31E−01 | 0.56 |
| SNED1 | 6.45 | 1.16E−09 | 0.51 | AARS | 4.07 | 2.68E−04 | 0.35 | ANXA7 | 2.82 | 1.96E−02 | 0.17 | ARHGDIB | 2.05 | 1.31E−01 | 0.51 |
| ABCB9 | 6.44 | 1.21E−09 | 1.09 | LCMT1 | 4.07 | 2.73E−04 | 0.33 | FGF2 | 2.82 | 1.99E−02 | 0.03 | MRPL18 | 2.05 | 1.33E−01 | 0.42 |
| CLIC5 | 6.44 | 1.27E−09 | 0.78 | PLXDC2 | 4.07 | 2.71E−04 | 0.31 | ALG1 | 2.81 | 2.05E−02 | 0.69 | KLHDC3 | 2.05 | 1.32E−01 | 0.27 |
| GOLGA2 | 6.44 | 1.26E−09 | 0.45 | NINJ1 | 4.06 | 2.85E−04 | 1.54 | EMILIN1 | 2.81 | 2.07E−02 | 0.66 | SNAPC4 | 2.05 | 1.33E−01 | 0.21 |
| FBXO2 | 6.43 | 1.35E−09 | 3.12 | FBXO44 | 4.06 | 2.87E−04 | 1 | TRAPPC2L | 2.81 | 2.03E−02 | 0.58 | E2F4 | 2.05 | 1.31E−01 | 0.18 |
| CLIC1 | 6.42 | 1.42E−09 | 0.57 | C12orf65 | 4.06 | 2.79E−04 | 0.79 | ENKUR | 2.81 | 2.04E−02 | 0.31 | HMGN2 | 2.05 | 1.31E−01 | 0.18 |
| PPP1R2 | 6.42 | 1.42E−09 | 0.47 | C12orf76 | 4.06 | 2.78E−04 | 0.72 | ZNF623 | 2.81 | 2.04E−02 | 0.28 | GNA11 | 2.05 | 1.31E−01 | 0.17 |
| SERP1 | 6.41 | 1.53E−09 | 0.42 | LRRC59 | 4.06 | 2.83E−04 | 0.57 | GRN | 2.81 | 2.02E−02 | 0.24 | STT3A | 2.05 | 1.31E−01 | 0.17 |
| FSCN1 | 6.4 | 1.61E−09 | 0.75 | ABCC4 | 4.06 | 2.81E−04 | 0.45 | SCML1 | 2.81 | 2.03E−02 | 0.23 | SLC25A29 | 2.05 | 1.31E−01 | 0.16 |
| S100A6 | 6.4 | 1.56E−09 | 0.25 | SH3GL1 | 4.06 | 2.78E−04 | 0.38 | BCAT1 | 2.81 | 2.03E−02 | 0.03 | RECOL | 2.05 | 1.31E−01 | 0.15 |
| 9-Sep | 6.4 | 1.60E−09 | 0.19 | FLRT3 | 4.05 | 2.91E−04 | 1.06 | U91319.1 | 2.8 | 2.13E−02 | 1.18 | PRKAR1A | 2.05 | 1.32E−01 | 0.11 |
| DDRGK1 | 6.39 | 1.71E−09 | 1.04 | NDUFB11 | 4.05 | 2.98E−04 | 1 | PTGES | 2.8 | 2.08E−02 | 0.92 | DRC7 | 2.04 | 1.34E−01 | 2.16 |
| MECP2 | 6.39 | 1.70E−09 | 0.51 | GPR173 | 4.05 | 3.01E−04 | 0.92 | TLE3 | 2.8 | 2.10E−02 | 0.25 | C16orf91 | 2.04 | 1.34E−01 | 1.06 |
| EVA1C | 6.39 | 1.68E−09 | 0.32 | B3GAT2 | 4.05 | 2.92E−04 | 0.66 | USP37 | 2.8 | 2.08E−02 | 0.18 | FAT3 | 2.04 | 1.34E−01 | 0.8 |
| PTRH1 | 6.38 | 1.85E−09 | 1.24 | MYLK | 4.05 | 2.99E−04 | 0.48 | FAM126B | 2.8 | 2.11E−02 | 0.18 | C22orf23 | 2.04 | 1.34E−01 | 0.71 |
| FAM227A | 6.38 | 1.77E−09 | 0.92 | NQO1 | 4.05 | 2.93E−04 | 0.43 | AC073218.1 | 2.79 | 2.18E−02 | 3.2 | PARP16 | 2.04 | 1.33E−01 | 0.4 |
| ENDOV | 6.38 | 1.80E−09 | 0.81 | PTGS2 | 4.04 | 3.13E−04 | 0.66 | CLDN3 | 2.79 | 2.15E−02 | 1.43 | THRA | 2.04 | 1.35E−01 | 0.31 |
| PAQR8 | 6.37 | 1.86E−09 | 3.02 | TP53BP1 | 4.04 | 3.07E−04 | 0.28 | ZNF771 | 2.79 | 2.19E−02 | 1.12 | FXYD5 | 2.04 | 1.36E−01 | 0.31 |
| MMP24 | 6.37 | 1.86E−09 | 0.68 | NXN | 4.04 | 3.07E−04 | 0.22 | SLC7A8 | 2.79 | 2.18E−02 | 0.93 | DDX49 | 2.04 | 1.34E−01 | 0.27 |
| MDK | 6.37 | 1.93E−09 | 0.66 | EMC8 | 4.03 | 3.15E−04 | 0.51 | SESN2 | 2.79 | 2.15E−02 | 0.71 | PIN4 | 2.04 | 1.36E−01 | 0.18 |
| SNCG | 6.36 | 2.03E−09 | 1.51 | ORAOV1 | 4.03 | 3.23E−04 | 0.48 | CTSL | 2.79 | 2.17E−02 | 0.71 | NSUN7 | 2.04 | 1.34E−01 | 0.17 |
| OAS3 | 6.35 | 2.17E−09 | 1.38 | NCBP2 | 4.03 | 3.21E−04 | 0.47 | TMEM129 | 2.79 | 2.19E−02 | 0.68 | CARS2 | 2.04 | 1.35E−01 | 0.16 |
| PKN1 | 6.34 | 2.29E−09 | 0.52 | HSP90AB1 | 4.03 | 3.25E−04 | 0.33 | WDR62 | 2.79 | 2.15E−02 | 0.65 | MXRA5 | 2.04 | 1.34E−01 | 0.15 |
| ADAM12 | 6.33 | 2.53E−09 | 1.08 | MAPK14 | 4.03 | 3.21E−04 | 0.28 | BCKDK | 2.79 | 2.16E−02 | 0.58 | TRIB1 | 2.04 | 1.36E−01 | 0.11 |
| NDUFB9 | 6.33 | 2.44E−09 | 0.92 | TSPYL2 | 4.02 | 3.38E−04 | 0.8 | BCORL1 | 2.79 | 2.14E−02 | 0.5 | IL1RAPL1 | 2.04 | 1.36E−01 | 0.1 |
| ATP6V1G1 | 6.33 | 2.45E−09 | 0.87 | CCDC24 | 4.02 | 3.26E−04 | 0.5 | PACRG | 2.79 | 2.18E−02 | 0.37 | COX19 | 2.04 | 1.36E−01 | 0.08 |
| SLC12A7 | 6.33 | 2.44E−09 | 0.44 | GPR160 | 4.02 | 3.38E−04 | 0.46 | C5AR1 | 2.79 | 2.15E−02 | 0.26 | HSD11B1 | 2.03 | 1.39E−01 | 1.96 |
| DHRSX | 6.33 | 2.49E−09 | 0.39 | UBE3B | 4.02 | 3.36E−04 | 0.45 | LURAPIL | 2.79 | 2.18E−02 | 0.23 | RAB24 | 2.03 | 1.36E−01 | 1.13 |
| PEX5L | 6.32 | 2.56E−09 | 2.41 | CYB5R3 | 4.02 | 3.28E−04 | 0.38 | CRYAB | 2.78 | 2.20E−02 | 3.61 | TUBA8 | 2.03 | 1.39E−01 | 1.11 |
| RYR2 | 6.32 | 2.69E−09 | 0.86 | CBX3 | 4.02 | 3.27E−04 | 0.3 | RITA1 | 2.78 | 2.24E−02 | 1.56 | AC009299.3 | 2.03 | 1.36E−01 | 0.99 |
| EIF3K | 6.32 | 2.57E−09 | 0.65 | LIPE-AS1 | 4.01 | 3.50E−04 | 0.36 | ANKRD1 | 2.78 | 2.23E−02 | 1.45 | COL14A1 | 2.03 | 1.36E−01 | 0.67 |
| SH2B3 | 6.31 | 2.81E−09 | 1.09 | TRPM4 | 4.01 | 3.45E−04 | 0.35 | RBFOX3 | 2.78 | 2.20E−02 | 1.37 | FAT4 | 2.03 | 1.37E−01 | 0.42 |
| SLC9A7 | 6.31 | 2.78E−09 | 0.55 | BEAN1 | 4.01 | 3.46E−04 | 0.32 | KCNAB2 | 2.78 | 2.23E−02 | 0.66 | NME4 | 2.03 | 1.36E−01 | 0.36 |
| GCSH | 6.3 | 2.98E−09 | 2.27 | TOR4A | 4 | 3.63E−04 | 0.83 | PLB1 | 2.78 | 2.21E−02 | 0.55 | PINK1 | 2.03 | 1.37E−01 | 0.33 |
| MZT2A | 6.3 | 3.05E−09 | 0.79 | ZNF213-AS1 | 4 | 3.54E−04 | 0.72 | ATP6V0A2 | 2.78 | 2.25E−02 | 0.32 | PUS1 | 2.03 | 1.39E−01 | 0.3 |
| EHD1 | 6.3 | 2.90E−09 | 0.74 | DNASE1L1 | 4 | 3.57E−04 | 0.67 | SYAP1 | 2.78 | 2.22E−02 | 0.25 | TBC1D10A | 2.03 | 1.37E−01 | 0.28 |
| CTSA | 6.3 | 2.97E−09 | 0.68 | ATP6V1E1 | 4 | 3.58E−04 | 0.41 | WDR73 | 2.78 | 2.21E−02 | 0.23 | TCHP | 2.03 | 1.38E−01 | 0.25 |
| PHLDB1 | 6.3 | 2.91E−09 | 0.6 | PLEKHB2 | 4 | 3.62E−04 | 0.28 | CMPK2 | 2.77 | 2.30E−02 | 1.73 | NLRP1 | 2.03 | 1.38E−01 | 0.19 |
| RPL29 | 6.3 | 2.88E−09 | 0.54 | 10-Mar | 3.99 | 3.80E−04 | 1.71 | RBP1 | 2.77 | 2.32E−02 | 0.85 | GBP3 | 2.03 | 1.39E−01 | 0.17 |
| HNRNPL | 6.3 | 2.89E−09 | 0.35 | RILPL2 | 3.99 | 3.71E−04 | 0.79 | MED19 | 2.77 | 2.30E−02 | 0.64 | UNC13D | 2.03 | 1.37E−01 | 0.14 |
| CLIC6 | 6.29 | 3.20E−09 | 1.12 | RMDN3 | 3.99 | 3.71E−04 | 0.51 | SPESP1 | 2.77 | 2.26E−02 | 0.62 | TMEM59 | 2.03 | 1.38E−01 | 0.14 |
| SPOCK1 | 6.29 | 3.11E−09 | 0.8 | CCDC65 | 3.99 | 3.72E−04 | 0.51 | NIT2 | 2.77 | 2.27E−02 | 0.54 | ATG13 | 2.03 | 1.38E−01 | 0.13 |
| SPDYE2 | 6.29 | 3.16E−09 | 0.62 | KXD1 | 3.99 | 3.78E−04 | 0.41 | REC8 | 2.77 | 2.31E−02 | 0.51 | CPEB1 | 2.02 | 1.41E−01 | 1.47 |
| YIPF2 | 6.28 | 3.45E−09 | 0.99 | LTBP4 | 3.99 | 3.80E−04 | 0.39 | MTHFD2 | 2.77 | 2.26E−02 | 0.49 | COPZ2 | 2.02 | 1.40E−01 | 1.3 |
| TRIM54 | 6.27 | 3.50E−09 | 1.81 | VWA3A | 3.98 | 3.87E−04 | 1.66 | GPS1 | 2.77 | 2.32E−02 | 0.4 | NPIPB6 | 2.02 | 1.41E−01 | 1.22 |
| ACHE | 6.27 | 3.67E−09 | 1.3 | CD163L1 | 3.98 | 3.85E−04 | 0.74 | AC091153.4 | 2.76 | 2.32E−02 | 3.71 | TEX11 | 2.02 | 1.39E−01 | 0.99 |
| CNTN4 | 6.27 | 3.57E−09 | 1.18 | HADHA | 3.98 | 3.89E−04 | 0.25 | KCNH3 | 2.76 | 2.32E−02 | 1.83 | PPP1R1B | 2.02 | 1.41E−01 | 0.83 |
| SPDYE1 | 6.27 | 3.62E−09 | 1.13 | SSSCA1 | 3.97 | 4.13E−04 | 1.39 | TSPAN10 | 2.76 | 2.35E−02 | 1.24 | WSCD1 | 2.02 | 1.40E−01 | 0.82 |
| RNF4 | 6.27 | 3.58E−09 | 0.45 | CHCHD5 | 3.97 | 4.05E−04 | 1.3 | KBTBD4 | 2.76 | 2.36E−02 | 1.12 | FSTL5 | 2.02 | 1.40E−01 | 0.72 |
| GREM1 | 6.26 | 3.85E−09 | 1.41 | KCNAB1 | 3.97 | 4.06E−04 | 1.14 | MERTK | 2.76 | 2.36E−02 | 0.89 | BLOC1S4 | 2.02 | 1.40E−01 | 0.57 |
| ARSG | 6.26 | 3.84E−09 | 0.76 | MEMO1 | 3.97 | 4.08E−04 | 0.64 | ACP2 | 2.76 | 2.38E−02 | 0.86 | MXRA7 | 2.02 | 1.40E−01 | 0.38 |
| MMP7 | 6.26 | 3.71E−09 | 0.7 | HSCB | 3.97 | 4.03E−04 | 0.57 | SORCS1 | 2.76 | 2.35E−02 | 0.79 | PCYOX1 | 2.02 | 1.40E−01 | 0.38 |
| CYB5B | 6.26 | 3.84E−09 | 0.47 | ACSF2 | 3.97 | 4.08E−04 | 0.36 | ATP12A | 2.76 | 2.33E−02 | 0.77 | MRPS2 | 2.02 | 1.41E−01 | 0.35 |
| ARMC8 | 6.26 | 3.85E−09 | 0.36 | SYN3 | 3.96 | 4.27E−04 | 0.93 | GRB14 | 2.76 | 2.36E−02 | 0.44 | KCNIP4 | 2.02 | 1.40E−01 | 0.27 |
| ALDH1L1 | 6.25 | 4.05E−09 | 1.2 | VAMP5 | 3.96 | 4.30E−04 | 0.91 | POLA2 | 2.76 | 2.38E−02 | 0.35 | TCF7 | 2.02 | 1.39E−01 | 0.23 |
| CALM3 | 6.25 | 3.95E−09 | 1.12 | JOSD2 | 3.96 | 4.24E−04 | 0.75 | RNF121 | 2.76 | 2.32E−02 | 0.23 | PBXIP1 | 2.02 | 1.40E−01 | 0.22 |
| CHCHD10 | 6.25 | 3.94E−09 | 1.06 | RABEPK | 3.96 | 4.21E−04 | 0.5 | EML2 | 2.76 | 2.35E−02 | 0.21 | HNRNPA0 | 2.02 | 1.40E−01 | 0.17 |
| SFXN5 | 6.25 | 4.01E−09 | 0.79 | LAC007389.3 | 3.95 | 4.41E−04 | 2.86 | UNC79 | 2.75 | 2.39E−02 | 0.98 | ARHGAP29 | 2.02 | 1.40E−01 | 0.13 |
| NFAT5 | 6.24 | 4.23E−09 | 0.14 | KREMEN1 | 3.95 | 4.34E−04 | 0.54 | SHMT2 | 2.75 | 2.44E−02 | 0.85 | SFI1 | 2.02 | 1.40E−01 | 0.13 |
| LMTK3 | 6.23 | 4.70E−09 | 0.77 | LINC00884 | 3.94 | 4.52E−04 | 1.02 | LRRTM4 | 2.75 | 2.44E−02 | 0.77 | LINC00954 | 2.01 | 1.43E−01 | 1.88 |
| ULK1 | 6.22 | 5.00E−09 | 0.53 | EPHX1 | 3.94 | 4.51E−04 | 0.72 | DIRC3 | 2.75 | 2.41E−02 | 0.72 | NELL1 | 2.01 | 1.44E−01 | 1.21 |
| ZXDC | 6.22 | 4.85E−09 | 0.45 | PSMD8 | 3.94 | 4.54E−04 | 0.5 | NDUFB3 | 2.75 | 2.43E−02 | 0.53 | SPSB2 | 2.01 | 1.42E−01 | 0.59 |
| SUPT5H | 6.22 | 5.00E−09 | 0.44 | ZNF526 | 3.93 | 4.75E−04 | 1.15 | CBR3 | 2.75 | 2.42E−02 | 0.52 | MRPL37 | 2.01 | 1.42E−01 | 0.5 |
| RAD23A | 6.21 | 5.24E−09 | 0.55 | OPCML | 3.93 | 4.73E−04 | 0.98 | OGFOD1 | 2.75 | 2.39E−02 | 0.32 | PEX16 | 2.01 | 1.42E−01 | 0.48 |
| PURA | 6.21 | 5.27E−09 | 0.47 | GTF2H5 | 3.93 | 4.81E−04 | 0.89 | MRPL19 | 2.75 | 2.44E−02 | 0.32 | PRDX3 | 2.01 | 1.44E−01 | 0.42 |
| C12orf57 | 6.2 | 5.56E−09 | 1.47 | MBD3 | 3.93 | 4.69E−04 | 0.57 | HECW2 | 2.75 | 2.41E−02 | 0.26 | ZP3 | 2.01 | 1.44E−01 | 0.36 |
| WDR66 | 6.2 | 5.50E−09 | 1 | DNAH3 | 3.93 | 4.83E−04 | 0.57 | DAP | 2.75 | 2.39E−02 | 0.24 | IL1R1 | 2.01 | 1.44E−01 | 0.27 |
| PES1 | 6.19 | 5.99E−09 | 0.84 | CA5B | 3.93 | 4.83E−04 | 0.34 | MAP3K3 | 2.75 | 2.43E−02 | 0.23 | STARD3NL | 2.01 | 1.44E−01 | 0.26 |
| LAMP1 | 6.19 | 6.01E−09 | 0.69 | SPTBN4 | 3.92 | 4.95E−04 | 1.21 | MAD1L1 | 2.75 | 2.38E−02 | 0.2 | SNRNP40 | 2.01 | 1.42E−01 | 0.2 |
| CHCHD6 | 6.19 | 5.77E−09 | 0.64 | HSBP1L1 | 3.92 | 5.04E−04 | 0.58 | TMEM67 | 2.75 | 2.45E−02 | 0.2 | STK11 | 2.01 | 1.44E−01 | 0.16 |
| MME | 6.18 | 6.36E−09 | 1.38 | SPON2 | 3.92 | 4.87E−04 | 0.5 | RPH3A | 2.74 | 2.46E−02 | 1.66 | TULP4 | 2.01 | 1.43E−01 | 0.13 |
| SGSM3 | 6.18 | 6.23E−09 | 0.63 | CAPN15 | 3.92 | 4.99E−04 | 0.41 | LINC01470 | 2.74 | 2.49E−02 | 0.95 | ELDR | 2 | 1.46E−01 | 1.28 |
| | | | | | | | | | | | | CIART | 2 | 1.45E−01 | 0.87 |
| | | | | | | | | | | | | TMEM136 | 2 | 1.46E−01 | 0.82 |
| | | | | | | | | | | | | DHCR7 | 2 | 1.46E−01 | 0.7 |
| | | | | | | | | | | | | WDR5B | 2 | 1.47E−01 | 0.68 |
| | | | | | | | | | | | | TMEM19 | 2 | 1.47E−01 | 0.64 |
| | | | | | | | | | | | | ALG12 | 2 | 1.47E−01 | 0.55 |
| | | | | | | | | | | | | HMBS | 2 | 1.46E−01 | 0.52 |
| | | | | | | | | | | | | ERG | 2 | 1.46E−01 | 0.5 |
| | | | | | | | | | | | | CDK2AP2 | 2 | 1.46E−01 | 0.47 |
| | | | | | | | | | | | | FAM66D | 2 | 1.46E−01 | 0.44 |
| | | | | | | | | | | | | EREG | 2 | 1.47E−01 | 0.3 |
| | | | | | | | | | | | | COPS7A | 2 | 1.46E−01 | 0.26 |
| | | | | | | | | | | | | SRSF8 | 2 | 1.46E−01 | 0.21 |
| | | | | | | | | | | | | CDK9 | 2 | 1.45E−01 | 0.19 |
| | | | | | | | | | | | | RARG | 2 | 1.46E−01 | 0.18 |
| | | | | | | | | | | | | BRK1 | 2 | 1.47E−01 | 0.15 |
| | | | | | | | | | | | | CALU | 2 | 1.45E−01 | 0.11 |
| | | | | | | | | | | | | GCLC | 2 | 1.45E−01 | 0.08 |

Abbreviations: FC = fold change

TABLE 2.3
CD8 by treatment

| Gene | Score | Pval | Log₂ FC | Gene | Score | Pval | Log₂ FC | Gene | Score | Pval | Log₂ FC | Gene | Score | Pval | Log₂ FC |
|---|---|---|---|---|---|---|---|---|---|---|---|---|---|---|---|
| PARP8 | 14.69 | 1.57E−44 | 1.46 | ARID1B | 3.85 | 0.0097 | 0.52 | CD8B | 2.86 | 0.1642 | 1.07 | TRPM3 | 2.34 | 0.504 | 3.43 |
| TC2N | 13.88 | 8.97E−40 | 2.22 | PPP1R12A | 3.84 | 0.0098 | 0.69 | LRRC37B | 2.86 | 0.1631 | 0.95 | OPRM1 | 2.34 | 0.4996 | 1.18 |
| IQGAP2 | 12.14 | 4.86E−30 | 1.68 | RORA | 3.84 | 0.0099 | 0.65 | CEP120 | 2.86 | 0.1631 | 0.81 | SRGAP2C | 2.34 | 0.504 | 0.95 |
| PDE7A | 10.78 | 2.28E−23 | 1.57 | HECA | 3.81 | 0.0109 | 0.99 | SP110 | 2.86 | 0.1651 | 0.61 | STAU2 | 2.34 | 0.504 | 0.56 |
| ETS1 | 10.73 | 3.34E−23 | 1.42 | PBX4 | 3.81 | 0.011 | 0.77 | ZNF148 | 2.86 | 0.1651 | 0.55 | MAP4 | 2.34 | 0.4996 | 0.41 |
| PPP2R5C | 10.67 | 5.25E−23 | 1.36 | IL7R | 3.79 | 0.012 | 0.8 | COG5 | 2.86 | 0.1641 | 0.51 | ANKRD26 | 2.33 | 0.5116 | 1.12 |
| CCND3 | 10.29 | 2.53E−21 | 1.25 | SNX25 | 3.78 | 0.0121 | 1.01 | EVL | 2.86 | 0.1637 | 0.45 | ABAT | 2.33 | 0.514 | 1.09 |
| CAMK4 | 9.78 | 3.66E−19 | 1.4 | JAML | 3.78 | 0.0122 | 0.82 | ANTXR2 | 2.85 | 0.1679 | 0.78 | DECR1 | 2.33 | 0.5175 | 0.62 |
| FAM129A | 9.47 | 5.49E−18 | 1.37 | DENND4A | 3.78 | 0.0121 | 0.65 | R3HDM2 | 2.85 | 0.1683 | 0.52 | ANKS1A | 2.33 | 0.514 | 0.52 |
| IKZF1 | 9.14 | 1.12E−16 | 1.2 | KIAA1551 | 3.78 | 0.0122 | 0.62 | CA5B | 2.84 | 0.1733 | 0.98 | LONP2 | 2.33 | 0.5131 | 0.48 |
| AOAH | 9.07 | 1.95E−16 | 1.3 | SIK3 | 3.78 | 0.0122 | 0.55 | ITGB1 | 2.84 | 0.171 | 0.62 | DENND1B | 2.33 | 0.5131 | 0.45 |
| DOCK10 | 9.06 | 2.07E−16 | 1.2 | YPEL1 | 3.77 | 0.0123 | 1.2 | MALT1 | 2.84 | 0.1726 | 0.6 | LCK | 2.33 | 0.5149 | 0.43 |
| PTPRC | 9.05 | 2.07E−16 | 0.76 | FGFR1OP2 | 3.77 | 0.0123 | 1.03 | DNASE1 | 2.83 | 0.1782 | 0.79 | ZNF217 | 2.32 | 0.5241 | 0.9 |
| FYN | 8.88 | 9.09E−16 | 0.86 | CCDC66 | 3.77 | 0.0124 | 0.91 | KIAA2026 | 2.83 | 0.1764 | 0.56 | TMEM71 | 2.32 | 0.5256 | 0.66 |
| PIP4K2A | 8.83 | 1.39E−15 | 1 | SORL1 | 3.77 | 0.0124 | 0.79 | XRCC5 | 2.83 | 0.1793 | 0.55 | NMT1 | 2.32 | 0.5241 | 0.65 |
| EMB | 8.75 | 2.74E−15 | 1.3 | PPP6R3 | 3.75 | 0.0133 | 0.66 | STK17B | 2.83 | 0.178 | 0.46 | PSPC1 | 2.32 | 0.522 | 0.45 |
| ANKRD44 | 8.62 | 7.78E−15 | 0.76 | SENP5 | 3.74 | 0.0135 | 0.93 | NLRC3 | 2.82 | 0.1799 | 0.69 | RBM26 | 2.32 | 0.5241 | 0.44 |
| ITGA4 | 8.56 | 1.27E−14 | 1.32 | EXOC6 | 3.74 | 0.0136 | 0.85 | SP140L | 2.82 | 0.1831 | 0.69 | GIGYF2 | 2.32 | 0.5241 | 0.43 |
| ATP8A1 | 8.54 | 1.38E−14 | 1.24 | ZFAND6 | 3.74 | 0.0136 | 0.74 | RPRD2 | 2.82 | 0.1799 | 0.54 | BTN3A1 | 2.32 | 0.5241 | 0.43 |
| SAMD3 | 8.36 | 6.07E−14 | 1.43 | SLC9A9 | 3.74 | 0.0135 | 0.72 | RALGAPB | 2.81 | 0.1845 | 0.61 | USP3 | 2.32 | 0.5241 | 0.42 |
| RASA3 | 8.22 | 1.82E−13 | 1.6 | ZZEF1 | 3.73 | 0.0142 | 0.77 | PPP4R3A | 2.81 | 0.1851 | 0.55 | DLG2 | 2.31 | 0.5275 | 0.81 |
| SLC38A1 | 8.16 | 2.91E−13 | 1.23 | TNFAIP3 | 3.73 | 0.0142 | 0.77 | PPP3CC | 2.81 | 0.1849 | 0.5 | VPS41 | 2.31 | 0.5266 | 0.54 |
| TTC39C | 8.13 | 3.59E−13 | 1.34 | APBA2 | 3.73 | 0.0138 | 0.74 | PPP1R16B | 2.81 | 0.1868 | 0.46 | HCLS1 | 2.31 | 0.5275 | 0.52 |
| FOXN3 | 8.06 | 6.20E−13 | 0.88 | GPATCH2 | 3.71 | 0.015 | 1.07 | CHST12 | 2.8 | 0.1907 | 0.76 | PPP2R5A | 2.31 | 0.5351 | 0.47 |
| PITPNC1 | 8.05 | 6.78E−13 | 0.97 | ANKH | 3.71 | 0.0149 | 0.89 | GPBP1L1 | 2.8 | 0.1918 | 0.56 | SMARCC1 | 2.31 | 0.5275 | 0.44 |
| CBLB | 7.99 | 1.06E−12 | 0.9 | MVB12B | 3.71 | 0.0149 | 0.74 | GPD1L | 2.79 | 0.1934 | 1.27 | AGO3 | 2.31 | 0.5303 | 0.44 |
| ITK | 7.98 | 1.12E−12 | 1.27 | PLCL2 | 3.71 | 0.0149 | 0.68 | OXCT1 | 2.79 | 0.1963 | 0.68 | LPP | 2.31 | 0.5307 | 0.41 |
| PRKCQ | 7.94 | 1.44E−12 | 1.29 | RBL2 | 3.71 | 0.0149 | 0.64 | MBD5 | 2.79 | 0.1945 | 0.48 | SNTG1 | 2.3 | 0.5476 | 2.72 |
| CHST11 | 7.89 | 2.05E−12 | 0.67 | AL592183.1 | 3.7 | 0.0152 | 1.51 | CCL5 | 2.78 | 0.2021 | 0.76 | TRPM7 | 2.3 | 0.5379 | 0.43 |
| THEMIS | 7.67 | 1.13E−11 | 0.87 | JAK2 | 3.69 | 0.0157 | 0.93 | ASAP1 | 2.78 | 0.202 | 0.53 | GALNT10 | 2.3 | 0.5367 | 0.4 |
| XIST | 7.66 | 1.13E−11 | 2.21 | CEP85L | 3.68 | 0.0163 | 0.64 | FNDC3A | 2.78 | 0.2027 | 0.53 | RASGRF2 | 2.29 | 0.5562 | 0.89 |
| MGAT5 | 7.58 | 2.18E−11 | 1.21 | CAPZB | 3.67 | 0.0167 | 0.61 | LINC00243 | 2.77 | 0.2028 | 1.5 | SPATA5 | 2.29 | 0.5569 | 0.49 |
| ELMO1 | 7.49 | 4.12E−11 | 1.08 | PIK3C3 | 3.66 | 0.0179 | 0.93 | TNRC6C | 2.77 | 0.2033 | 0.54 | B4GALT5 | 2.29 | 0.5574 | 0.49 |
| CD96 | 7.38 | 9.13E−11 | 0.96 | ANXA6 | 3.65 | 0.0182 | 1.13 | R3HDM1 | 2.76 | 0.209 | 0.53 | XPO6 | 2.29 | 0.5574 | 0.45 |
| SYNE1 | 7.31 | 1.44E−10 | 1.12 | PAN3 | 3.65 | 0.0181 | 0.58 | GLIS3 | 2.75 | 0.2168 | 0.92 | PTPN1 | 2.29 | 0.5561 | 0.43 |
| FLI1 | 7.28 | 1.76E−10 | 1.18 | PRMT2 | 3.63 | 0.0196 | 0.76 | DIP2A | 2.75 | 0.2154 | 0.55 | IPO11 | 2.28 | 0.5695 | 1.05 |
| CNOT6L | 7.2 | 3.21E−10 | 1.06 | CDR2 | 3.63 | 0.0197 | 0.74 | TES | 2.74 | 0.2232 | 0.82 | AP2A2 | 2.28 | 0.5654 | 0.67 |
| SLC4A4 | 7.18 | 3.71E−10 | 2.37 | KDM5A | 3.63 | 0.0196 | 0.71 | KDM2B | 2.74 | 0.2244 | 0.56 | KIF21A | 2.28 | 0.5603 | 0.65 |
| WIPF1 | 7.14 | 4.72E−10 | 1.03 | TMEM181 | 3.63 | 0.0195 | 0.67 | ADD3 | 2.74 | 0.222 | 0.51 | FBXO42 | 2.28 | 0.5618 | 0.49 |
| SLFN12L | 7.13 | 4.88E−10 | 1.07 | CAMK2D | 3.63 | 0.0196 | 0.64 | RFX7 | 2.73 | 0.2305 | 0.49 | GLMN | 2.28 | 0.5669 | 0.47 |
| ZNF831 | 7.12 | 5.17E−10 | 1.35 | ATG16L1 | 3.62 | 0.0199 | 1 | BCR | 2.72 | 0.2347 | 0.78 | SPG11 | 2.28 | 0.5605 | 0.41 |
| STAT4 | 7.11 | 5.32E−10 | 0.98 | ARHGEF7 | 3.62 | 0.0199 | 0.67 | VPS13B | 2.72 | 0.2349 | 0.5 | ABCC1 | 2.28 | 0.5648 | 0.41 |
| PREX1 | 7.07 | 7.31E−10 | 1.25 | RFTN1 | 3.61 | 0.0204 | 0.68 | TTC17 | 2.72 | 0.2313 | 0.45 | CRIM1 | 2.27 | 0.5767 | 0.6 |
| SMCHD1 | 7.06 | 7.66E−10 | 0.87 | ATM | 3.61 | 0.0207 | 0.62 | CEL | 2.71 | 0.2393 | 4.36 | AFF4 | 2.27 | 0.5814 | 0.49 |
| PRKCH | 7.04 | 8.26E−10 | 0.62 | TRAPPC9 | 3.61 | 0.0207 | 0.62 | PIK3CB | 2.71 | 0.2377 | 0.62 | XYLT1 | 2.27 | 0.5775 | 0.42 |
| EML4 | 7.03 | 9.05E−10 | 1.02 | GPR171 | 3.6 | 0.0214 | 1.31 | TRIM14 | 2.71 | 0.2377 | 0.59 | AKAP7 | 2.26 | 0.5899 | 0.95 |
| FOXP1 | 6.85 | 3.14E−09 | 0.8 | CCM2 | 3.6 | 0.0214 | 0.66 | TMEM260 | 2.7 | 0.2446 | 0.81 | ZBTB44 | 2.26 | 0.5871 | 0.78 |
| SKAP1 | 6.81 | 4.11E−09 | 0.69 | RAB2A | 3.59 | 0.0218 | 0.66 | MAD1L1 | 2.7 | 0.2459 | 0.73 | DZIP3 | 2.26 | 0.5929 | 0.61 |
| PIK3R1 | 6.76 | 5.36E−09 | 1.2 | TDRD3 | 3.58 | 0.0225 | 0.85 | PEX14 | 2.7 | 0.2424 | 0.63 | RSU1 | 2.26 | 0.5858 | 0.55 |
| SLAMF6 | 6.74 | 5.87E−09 | 1.3 | GCG | 3.55 | 0.0254 | 3.08 | NLRP1 | 2.7 | 0.2433 | 0.52 | WDR70 | 2.26 | 0.5875 | 0.45 |
| CCNH | 6.74 | 5.91E−09 | 1.21 | COMMD10 | 3.54 | 0.0258 | 0.72 | UBL3 | 2.7 | 0.2456 | 0.51 | LRCH1 | 2.26 | 0.5921 | 0.41 |
| GRAP2 | 6.72 | 6.62E−09 | 1.1 | ATF7IP | 3.54 | 0.0261 | 0.62 | CD28 | 2.69 | 0.2476 | 0.96 | EXT2 | 2.25 | 0.6002 | 0.94 |
| NSF | 6.69 | 8.22E−09 | 1.2 | ERO1B | 3.53 | 0.027 | 1.11 | UBR4 | 2.69 | 0.2488 | 0.65 | VPS50 | 2.25 | 0.5962 | 0.68 |
| FAM214A | 6.66 | 9.52E−09 | 1.17 | DPP8 | 3.53 | 0.0266 | 0.86 | CARD8 | 2.69 | 0.2476 | 0.53 | RASSF1 | 2.25 | 0.6009 | 0.61 |
| CCSER2 | 6.66 | 9.78E−09 | 1.07 | TRAF3IP3 | 3.53 | 0.0264 | 0.68 | TRIM33 | 2.69 | 0.2476 | 0.53 | PBRM1 | 2.25 | 0.6009 | 0.44 |
| MBNL1 | 6.64 | 1.10E−08 | 0.59 | HUWE1 | 3.53 | 0.0264 | 0.67 | FTX | 2.69 | 0.2476 | 0.52 | MED13L | 2.25 | 0.6023 | 0.38 |
| SCML4 | 6.62 | 1.25E−08 | 1.18 | CCDC7 | 3.52 | 0.0275 | 0.87 | EP300 | 2.69 | 0.2476 | 0.51 | APMAP | 2.24 | 0.6113 | 0.82 |
| CYTIP | 6.57 | 1.67E−08 | 1.07 | PSME4 | 3.52 | 0.0273 | 0.66 | 7-Mar | 2.69 | 0.2488 | 0.51 | EDEM3 | 2.24 | 0.6108 | 0.62 |
| RBMS1 | 6.56 | 1.76E−08 | 1.04 | MAML2 | 3.52 | 0.0278 | 0.49 | ACAP2 | 2.69 | 0.2476 | 0.49 | PPP1CB | 2.24 | 0.6149 | 0.62 |
| DOCK2 | 6.52 | 2.26E−08 | 0.98 | SLC14A1 | 3.51 | 0.0285 | 1.87 | BTG1 | 2.69 | 0.2506 | 0.46 | DDX10 | 2.24 | 0.6074 | 0.59 |
| ARHGEF3 | 6.5 | 2.51E−08 | 1.02 | HELZ | 3.51 | 0.0285 | 0.66 | MYCBP2 | 2.69 | 0.2476 | 0.44 | SRBD1 | 2.24 | 0.6108 | 0.54 |
| SMAP2 | 6.48 | 2.82E−08 | 1.08 | GLG1 | 3.51 | 0.0282 | 0.62 | ATXN10 | 2.68 | 0.254 | 0.5 | TBC1D14 | 2.24 | 0.6058 | 0.52 |
| ST6GAL1 | 6.46 | 3.28E−08 | 1.18 | KDM4C | 3.51 | 0.0286 | 0.62 | RHOH | 2.68 | 0.2514 | 0.47 | KLHDC4 | 2.24 | 0.6059 | 0.45 |
| ARHGAP15 | 6.39 | 5.08E−08 | 0.56 | FGD3 | 3.5 | 0.029 | 0.71 | EPS15 | 2.68 | 0.2524 | 0.47 | TRPC4AP | 2.24 | 0.6059 | 0.44 |
| INPP5D | 6.33 | 7.28E−08 | 0.99 | SENP7 | 3.5 | 0.0289 | 0.64 | SNHG22 | 2.67 | 0.2583 | 1.37 | BDP1 | 2.24 | 0.6108 | 0.43 |
| KAT2B | 6.32 | 7.97E−08 | 1.1 | AHCYL2 | 3.49 | 0.0304 | 0.86 | CRBN | 2.67 | 0.2583 | 0.92 | HS6ST3 | 2.23 | 0.6152 | 2.56 |
| RAP1GDS1 | 6.32 | 7.81E−08 | 1.04 | EXOC2 | 3.49 | 0.03 | 0.71 | TARSL2 | 2.67 | 0.2583 | 0.71 | TBC1D22B | 2.23 | 0.6231 | 1.06 |
| DOCK8 | 6.28 | 9.81E−08 | 0.8 | FBXW4 | 3.49 | 0.0301 | 0.71 | FBXL20 | 2.67 | 0.2583 | 0.52 | EIF4E3 | 2.23 | 0.6176 | 0.7 |
| RNF125 | 6.22 | 1.45E−07 | 1.39 | ITPR1 | 3.49 | 0.0297 | 0.67 | GRK5 | 2.67 | 0.2586 | 0.51 | CNST | 2.23 | 0.6179 | 0.59 |
| CDC14A | 6.19 | 1.67E−07 | 1.1 | RIMS2 | 3.48 | 0.0308 | 2.4 | PDE3A | 2.66 | 0.2635 | 1.31 | SLC25A26 | 2.23 | 0.6168 | 0.58 |
| AUTS2 | 6.1 | 3.02E−07 | 1.08 | HIBCH | 3.48 | 0.0309 | 0.82 | RGS1 | 2.66 | 0.2677 | 0.61 | USP10 | 2.23 | 0.6231 | 0.54 |
| CAMK1D | 6.08 | 3.27E−07 | 1.17 | RNF149 | 3.48 | 0.0309 | 0.62 | SEL1L3 | 2.66 | 0.2643 | 0.53 | TACC1 | 2.23 | 0.6221 | 0.45 |
| TTC39B | 6.05 | 3.97E−07 | 1.24 | THADA | 3.47 | 0.0315 | 0.66 | YTHDC1 | 2.66 | 0.2635 | 0.5 | FCHSD2 | 2.23 | 0.6231 | 0.41 |
| ZFP36L2 | 6 | 5.21E−07 | 1.01 | CRTC3 | 3.45 | 0.0335 | 0.73 | FARS2 | 2.66 | 0.2644 | 0.5 | DPP6 | 2.22 | 0.6336 | 1.52 |
| ATF7IP2 | 5.92 | 8.58E−07 | 1.22 | DCAF5 | 3.44 | 0.035 | 0.74 | PIAS2 | 2.65 | 0.2685 | 0.88 | GBP5 | 2.22 | 0.6294 | 0.63 |
| PDE3B | 5.9 | 9.36E−07 | 0.83 | SCAF8 | 3.44 | 0.0351 | 0.65 | ZNF827 | 2.65 | 0.2732 | 0.83 | NIPAL3 | 2.22 | 0.6281 | 0.62 |
| HERC1 | 5.88 | 1.04E−06 | 0.93 | ADARB1 | 3.42 | 0.037 | 1.11 | USP28 | 2.65 | 0.2723 | 0.76 | ANO6 | 2.22 | 0.6282 | 0.59 |
| UTRN | 5.87 | 1.15E−06 | 0.71 | GIT2 | 3.42 | 0.0374 | 0.75 | PCCA | 2.65 | 0.2734 | 0.52 | MAU2 | 2.22 | 0.6301 | 0.58 |
| PIK3R5 | 5.79 | 1.82E−06 | 0.97 | ASCC3 | 3.42 | 0.037 | 0.62 | PTPRA | 2.65 | 0.2685 | 0.49 | MBTPS1 | 2.22 | 0.6328 | 0.56 |
| SUCLG2 | 5.75 | 2.31E−06 | 0.94 | RAP1A | 3.42 | 0.0371 | 0.56 | TANK | 2.65 | 0.2739 | 0.48 | ACBD6 | 2.22 | 0.6319 | 0.49 |
| TNFAIP8 | 5.74 | 2.37E−06 | 0.9 | CD44 | 3.42 | 0.0374 | 0.54 | CPT1A | 2.64 | 0.2793 | 1.05 | PSMA1 | 2.22 | 0.6355 | 0.46 |
| BCL11B | 5.71 | 2.74E−06 | 0.87 | MAP2K6 | 3.41 | 0.0387 | 1.03 | HERC4 | 2.64 | 0.2758 | 0.49 | CADPS | 2.21 | 0.6408 | 1.47 |
| APBB1IP | 5.7 | 2.95E−06 | 0.91 | TMEM156 | 3.39 | 0.0406 | 0.77 | UMAD1 | 2.64 | 0.281 | 0.47 | CLNS1A | 2.21 | 0.6368 | 0.91 |
| SAMHD1 | 5.69 | 3.09E−06 | 1.24 | TAF3 | 3.39 | 0.0415 | 0.7 | FCMR | 2.63 | 0.2824 | 1.3 | ELOVL6 | 2.21 | 0.6368 | 0.9 |
| DIAPH1 | 5.68 | 3.25E−06 | 1.01 | SYTL2 | 3.39 | 0.0407 | 0.64 | ADCY9 | 2.63 | 0.282 | 1.18 | ZNF254 | 2.21 | 0.6395 | 0.64 |
| CDC42SE2 | 5.67 | 3.38E−06 | 0.74 | FBXL17 | 3.39 | 0.0406 | 0.54 | ADSS | 2.63 | 0.2852 | 0.7 | CASC4 | 2.21 | 0.6453 | 0.45 |
| MDFIC | 5.66 | 3.52E−06 | 1.02 | NT5DC1 | 3.38 | 0.0415 | 0.91 | NSMAF | 2.63 | 0.2821 | 0.62 | GPBP1 | 2.21 | 0.6423 | 0.43 |
| OXNAD1 | 5.65 | 3.56E−06 | 0.96 | DNAJB6 | 3.38 | 0.0417 | 0.73 | ABHD18 | 2.63 | 0.2855 | 0.56 | NUP210 | 2.21 | 0.6433 | 0.43 |
| SOS1 | 5.65 | 3.56E−06 | 0.95 | MECP2 | 3.38 | 0.0421 | 0.63 | UBASH3A | 2.62 | 0.2885 | 0.85 | SMARCA2 | 2.21 | 0.6393 | 0.41 |
| LEPROTL1 | 5.58 | 5.25E−06 | 1.11 | C12orf75 | 3.37 | 0.0427 | 1.2 | CLINT1 | 2.62 | 0.2864 | 0.59 | GPATCH8 | 2.21 | 0.6443 | 0.36 |
| PRKCB | 5.53 | 6.96E−06 | 0.82 | FAM193A | 3.36 | 0.0441 | 0.82 | RUNX2 | 2.62 | 0.29 | 0.54 | RNF157 | 2.2 | 0.6595 | 0.56 |
| NCOA2 | 5.52 | 7.53E−06 | 0.88 | ZNF131 | 3.36 | 0.045 | 0.68 | TOX | 2.62 | 0.2872 | 0.34 | DOPEY1 | 2.2 | 0.6528 | 0.53 |
| TGFBR3 | 5.5 | 8.33E−06 | 1.4 | NR3C2 | 3.35 | 0.0456 | 1.01 | ZNF385D | 2.61 | 0.2965 | 1.54 | WAPL | 2.2 | 0.6543 | 0.44 |
| STK4 | 5.5 | 8.13E−06 | 0.87 | ARHGDIB | 3.35 | 0.0465 | 0.79 | SLC10A7 | 2.61 | 0.2975 | 0.73 | WDTC1 | 2.19 | 0.6631 | 0.93 |
| HELB | 5.48 | 9.21E−06 | 1.29 | TTN | 3.34 | 0.0479 | 0.92 | SFSWAP | 2.61 | 0.2965 | 0.66 | NCOA1 | 2.19 | 0.6665 | 0.41 |
| LINC00623 | 5.47 | 9.47E−06 | 0.96 | ATP10A | 3.34 | 0.0474 | 0.72 | XPR1 | 2.61 | 0.2932 | 0.51 | NFATC3 | 2.19 | 0.666 | 0.4 |
| TBC1D1 | 5.46 | 1.00E−05 | 1.02 | FAM107B | 3.34 | 0.0474 | 0.65 | XKR6 | 2.61 | 0.2987 | 0.49 | MBNL2 | 2.19 | 0.6684 | 0.38 |
| STK10 | 5.39 | 1.48E−05 | 0.88 | RASSF3 | 3.33 | 0.0484 | 0.66 | RAP1B | 2.61 | 0.2932 | 0.48 | ARRDC3 | 2.18 | 0.6871 | 0.7 |
| PACS1 | 5.39 | 1.48E−05 | 0.77 | ZFYVE28 | 3.33 | 0.0493 | 0.6 | TTN-AS1 | 2.6 | 0.302 | 0.68 | MAX | 2.18 | 0.677 | 0.48 |
| ATP2B1 | 5.37 | 1.61E−05 | 1.15 | TCF12 | 3.33 | 0.0484 | 0.59 | CLEC16A | 2.6 | 0.3034 | 0.62 | ANAPC16 | 2.18 | 0.6764 | 0.44 |
| ARHGAP35 | 5.37 | 1.61E−05 | 1.03 | NR3C1 | 3.33 | 0.0491 | 0.58 | CAB39 | 2.6 | 0.302 | 0.49 | TESPA1 | 2.17 | 0.6938 | 1 |
| ZC3HAV1 | 5.37 | 1.61E−05 | 0.85 | SNTB1 | 3.32 | 0.0495 | 0.84 | TAOK3 | 2.6 | 0.3044 | 0.46 | PXN | 2.17 | 0.7023 | 0.73 |
| PYHIN1 | 5.36 | 1.62E−05 | 0.92 | MAN2A1 | 3.32 | 0.0495 | 0.66 | RALYL | 2.59 | 0.3084 | 2.73 | EEFSEC | 2.17 | 0.7018 | 0.61 |
| PDCD4 | 5.34 | 1.86E−05 | 0.91 | CCDC88C | 3.32 | 0.0504 | 0.58 | ARFIP1 | 2.59 | 0.3068 | 1.06 | SETD2 | 2.17 | 0.7021 | 0.43 |
| TSPAN5 | 5.31 | 2.10E−05 | 0.92 | DUSP16 | 3.31 | 0.0511 | 0.66 | MAGI2 | 2.59 | 0.3072 | 1.06 | MTERF1 | 2.16 | 0.7048 | 0.91 |
| MYBL1 | 5.3 | 2.18E−05 | 1.53 | BRWD1 | 3.31 | 0.0516 | 0.57 | KCNAB2 | 2.59 | 0.3072 | 0.52 | PARVG | 2.16 | 0.708 | 0.75 |
| NCK2 | 5.3 | 2.27E−05 | 0.92 | C12orf42 | 3.3 | 0.0533 | 1.5 | ARID5B | 2.59 | 0.311 | 0.42 | PMS1 | 2.16 | 0.7112 | 0.61 |
| CD69 | 5.29 | 2.36E−05 | 1.06 | WDR7 | 3.3 | 0.053 | 0.65 | UBA6-AS1 | 2.58 | 0.3143 | 1.38 | DISC1 | 2.16 | 0.711 | 0.59 |
| ATXN1 | 5.28 | 2.41E−05 | 0.45 | RPS6KA5 | 3.29 | 0.0554 | 0.68 | CAMK2G | 2.58 | 0.3154 | 0.86 | EVA1C | 2.16 | 0.7062 | 0.58 |
| RALGAPA1 | 5.25 | 2.79E−05 | 0.9 | GNAS | 3.29 | 0.0555 | 0.62 | KLF13 | 2.58 | 0.3167 | 0.68 | ERICH1 | 2.16 | 0.7101 | 0.48 |
| TGFBR2 | 5.23 | 3.11E−05 | 0.82 | RAB30-AS1 | 3.28 | 0.0574 | 1.46 | DENND2D | 2.58 | 0.3148 | 0.57 | CRLF3 | 2.16 | 0.708 | 0.4 |
| SEMA4D | 5.22 | 3.34E−05 | 0.87 | TRIM22 | 3.28 | 0.0564 | 0.68 | PDE4DIP | 2.58 | 0.3159 | 0.55 | ASXL2 | 2.16 | 0.7098 | 0.4 |
| KAT6B | 5.18 | 3.97E−05 | 0.99 | N4BP2 | 3.27 | 0.0576 | 1.09 | RUBCN | 2.57 | 0.3208 | 0.93 | ADGRE5 | 2.16 | 0.708 | 0.36 |
| GABPB1-AS1 | 5.16 | 4.28E−05 | 1.16 | NPIPB5 | 3.27 | 0.0587 | 0.78 | ANKRD36B | 2.57 | 0.3244 | 0.85 | AVEN | 2.15 | 0.7217 | 1.3 |
| CD53 | 5.16 | 4.38E−05 | 0.99 | TMEM131 | 3.27 | 0.0576 | 0.65 | MRPL1 | 2.57 | 0.3198 | 0.81 | ABCD2 | 2.15 | 0.7196 | 1 |
| STK39 | 5.16 | 4.38E−05 | 0.85 | E2F3 | 3.26 | 0.0604 | 0.8 | ERC1 | 2.57 | 0.3204 | 0.53 | AMMECR1 | 2.15 | 0.7159 | 0.73 |
| MSH3 | 5.15 | 4.39E−05 | 1.29 | RAPGEF1 | 3.26 | 0.0604 | 0.52 | TOP2B | 2.57 | 0.323 | 0.5 | NFYC | 2.15 | 0.7196 | 0.67 |
| USP15 | 5.14 | 4.70E−05 | 0.85 | RANBP9 | 3.25 | 0.0622 | 0.65 | LCOR | 2.57 | 0.3231 | 0.48 | FBXO3 | 2.15 | 0.7152 | 0.66 |
| MACF1 | 5.14 | 4.78E−05 | 0.74 | STIM2 | 3.25 | 0.062 | 0.63 | ATAD2B | 2.57 | 0.3244 | 0.48 | ZC3H13 | 2.15 | 0.7217 | 0.48 |
| ARL15 | 5.06 | 7.00E−05 | 0.78 | TBCD | 3.25 | 0.0622 | 0.59 | STX18 | 2.56 | 0.3261 | 0.83 | SFI1 | 2.15 | 0.7136 | 0.43 |
| ARHGAP26 | 5.04 | 7.63E−05 | 0.78 | STAG1 | 3.25 | 0.0617 | 0.53 | PHF12 | 2.56 | 0.328 | 0.71 | CNOT2 | 2.15 | 0.7217 | 0.42 |
| GNG2 | 5.02 | 8.64E−05 | 0.83 | TRGC2 | 3.24 | 0.0629 | 1.11 | DEF6 | 2.56 | 0.3261 | 0.61 | XRN1 | 2.15 | 0.7217 | 0.42 |
| AC016831.7 | 5.02 | 8.67E−05 | 0.62 | PLXNC1 | 3.24 | 0.0637 | 1.09 | DGKD | 2.56 | 0.33 | 0.61 | GZMK | 2.14 | 0.7315 | 1.27 |
| WWP1 | 5.01 | 8.75E−05 | 0.99 | ZNF800 | 3.24 | 0.0634 | 0.9 | ZBTB38 | 2.56 | 0.3297 | 0.55 | ATG12 | 2.14 | 0.7328 | 0.7 |
| SLA2 | 4.99 | 9.54E−05 | 1.39 | APPL2 | 3.24 | 0.0634 | 0.87 | CDK17 | 2.56 | 0.3251 | 0.49 | DCAF10 | 2.14 | 0.7301 | 0.5 |
| TNIK | 4.95 | 0.00012 | 0.86 | YAF2 | 3.24 | 0.0629 | 0.7 | VPS13C | 2.56 | 0.3251 | 0.49 | SLC20A2 | 2.14 | 0.7288 | 0.45 |
| SYNRG | 4.95 | 0.00012 | 0.86 | ZCCHC7 | 3.24 | 0.0629 | 0.52 | GNA13 | 2.55 | 0.3372 | 0.61 | FAM102A | 2.14 | 0.7387 | 0.39 |
| ATG7 | 4.94 | 0.00012 | 0.9 | CPB1 | 3.23 | 0.0647 | 5.6 | LPXN | 2.55 | 0.3372 | 0.59 | CTRB1 | 2.13 | 0.7434 | 6.26 |
| VAV3 | 4.93 | 0.00013 | 0.92 | GNAO1 | 3.23 | 0.065 | 1.23 | MYO1F | 2.55 | 0.3382 | 0.47 | GIMAP2 | 2.13 | 0.7405 | 1.21 |
| LINC00426 | 4.9 | 0.00015 | 1.14 | SETD3 | 3.23 | 0.0647 | 1.01 | CD5 | 2.54 | 0.3416 | 1.97 | SLAIN2 | 2.13 | 0.7419 | 0.53 |
| GPRIN3 | 4.9 | 0.00015 | 0.86 | MFSD6 | 3.23 | 0.0647 | 0.73 | TCAIM | 2.54 | 0.3415 | 1.33 | TM9SF2 | 2.13 | 0.7405 | 0.45 |
| RASA2 | 4.88 | 0.00016 | 0.69 | CACNB2 | 3.22 | 0.0665 | 1.8 | PIBF1 | 2.54 | 0.3451 | 0.48 | SMG7 | 2.13 | 0.7405 | 0.44 |
| AAK1 | 4.87 | 0.00017 | 0.74 | MXD4 | 3.22 | 0.0664 | 1.11 | KIAA0513 | 2.53 | 0.3451 | 0.97 | PDSS2 | 2.13 | 0.7499 | 0.43 |
| SLC4A7 | 4.86 | 0.00018 | 0.82 | CLEC2B | 3.22 | 0.0662 | 0.87 | ASCC1 | 2.53 | 0.3491 | 0.81 | GON4L | 2.13 | 0.7405 | 0.42 |
| CMTM7 | 4.85 | 0.00018 | 1.14 | CREBRF | 3.22 | 0.0664 | 0.79 | RELL1 | 2.53 | 0.3451 | 0.8 | RAB3GAP2 | 2.13 | 0.7434 | 0.41 |
| CFTR | 4.82 | 0.00021 | 1.86 | KDM6A | 3.22 | 0.0662 | 0.56 | ARHGAP10 | 2.53 | 0.3451 | 0.77 | UBE2H | 2.13 | 0.7437 | 0.38 |
| USP25 | 4.82 | 0.00021 | 0.83 | CD8A | 3.21 | 0.0676 | 1.14 | CERK | 2.53 | 0.3451 | 0.73 | KCNB2 | 2.12 | 0.7523 | 3.12 |
| DYRK1A | 4.81 | 0.00023 | 0.79 | RBM5 | 3.21 | 0.0665 | 0.65 | PCAT1 | 2.53 | 0.3451 | 0.71 | EXOSC10 | 2.12 | 0.7505 | 1.1 |
| DNAJC1 | 4.8 | 0.00023 | 0.79 | SP140 | 3.21 | 0.0676 | 0.65 | NPAT | 2.53 | 0.3491 | 0.71 | LY9 | 2.12 | 0.7523 | 0.88 |
| BIN2 | 4.79 | 0.00024 | 1.12 | JPX | 3.21 | 0.0676 | 0.64 | CLYBL | 2.53 | 0.3491 | 0.63 | NHLRC2 | 2.12 | 0.7505 | 0.77 |
| STIM1 | 4.77 | 0.00027 | 0.84 | MAPKAPK2 | 3.2 | 0.0692 | 0.88 | UBAC2 | 2.53 | 0.3451 | 0.5 | CD3E | 2.12 | 0.7523 | 0.71 |
| TLE4 | 4.76 | 0.00027 | 1.01 | REV3L | 3.2 | 0.0686 | 0.64 | CLEC2D | 2.53 | 0.3451 | 0.46 | ANAPC4 | 2.12 | 0.7523 | 0.7 |
| PRKACB | 4.76 | 0.00027 | 0.93 | MGAT4A | 3.2 | 0.0678 | 0.59 | ABLIM1 | 2.53 | 0.3451 | 0.46 | ZNF276 | 2.12 | 0.7523 | 0.65 |
| SYTL3 | 4.76 | 0.00027 | 0.69 | ARHGAP25 | 3.2 | 0.0695 | 0.58 | KIF13B | 2.53 | 0.3451 | 0.45 | MTM1 | 2.12 | 0.7523 | 0.58 |
| PRKX | 4.73 | 0.00032 | 1.03 | FAM120B | 3.19 | 0.0717 | 0.66 | B4GALT1 | 2.52 | 0.3514 | 0.66 | SIK2 | 2.12 | 0.7523 | 0.5 |
| ELOVL5 | 4.73 | 0.00031 | 0.98 | RUNX3 | 3.19 | 0.0708 | 0.59 | TRAPPC12 | 2.52 | 0.3572 | 0.63 | NR2C2 | 2.12 | 0.7564 | 0.43 |
| GLCCI1 | 4.73 | 0.00031 | 0.85 | PIK3IP1 | 3.18 | 0.0724 | 0.71 | PDK4 | 2.51 | 0.3602 | 1.81 | CAPZA1 | 2.12 | 0.7523 | 0.39 |
| RPS6KA3 | 4.72 | 0.00032 | 0.76 | PCGF5 | 3.18 | 0.0719 | 0.62 | NMT2 | 2.51 | 0.3602 | 0.81 | CWF19L1 | 2.11 | 0.7692 | 0.9 |
| PDE4B | 4.71 | 0.00034 | 0.98 | AGTPBP1 | 3.18 | 0.0723 | 0.62 | USP9X | 2.51 | 0.3624 | 0.48 | PLEKHG1 | 2.11 | 0.7706 | 0.67 |
| STK17A | 4.7 | 0.00036 | 0.82 | ABI1 | 3.18 | 0.0724 | 0.6 | RNF115 | 2.51 | 0.3602 | 0.47 | LPGAT1 | 2.11 | 0.7692 | 0.46 |
| LCP1 | 4.65 | 0.00045 | 0.89 | CCNY | 3.18 | 0.0719 | 0.59 | ABR | 2.51 | 0.3608 | 0.47 | HECTD4 | 2.11 | 0.7692 | 0.4 |
| CMIP | 4.65 | 0.00045 | 0.74 | NRXN1 | 3.17 | 0.0752 | 2.39 | SON | 2.51 | 0.3622 | 0.46 | ITSN2 | 2.11 | 0.7602 | 0.39 |
| PLEKHA2 | 4.64 | 0.00045 | 0.91 | UBE2D3 | 3.17 | 0.0752 | 0.59 | ERAP2 | 2.51 | 0.3628 | 0.45 | PICALM | 2.11 | 0.7692 | 0.39 |
| ITFG1 | 4.64 | 0.00047 | 0.81 | NCOR1 | 3.17 | 0.0745 | 0.57 | PTPRN2 | 2.5 | 0.3652 | 1.57 | GGA2 | 2.11 | 0.773 | 0.39 |
| AKNA | 4.61 | 0.00053 | 0.82 | FAM172A | 3.17 | 0.0749 | 0.51 | GLIPR1 | 2.5 | 0.365 | 1.25 | MPC1 | 2.1 | 0.7754 | 1.3 |
| LINC01004 | 4.6 | 0.00056 | 1.23 | BRD1 | 3.16 | 0.0774 | 0.73 | TMEM161B-AS1 | 2.5 | 0.3714 | 0.77 | F2R | 2.1 | 0.7754 | 1.17 |
| SPOCK2 | 4.59 | 0.00058 | 1.23 | COA1 | 3.15 | 0.0783 | 0.63 | GSE1 | 2.5 | 0.365 | 0.67 | LRIG1 | 2.1 | 0.7819 | 0.78 |
| FAM69A | 4.55 | 0.00068 | 0.9 | SDK1 | 3.14 | 0.0817 | 1.24 | DGKA | 2.5 | 0.3719 | 0.54 | ABCC4 | 2.1 | 0.7745 | 0.77 |
| LPIN2 | 4.55 | 0.00069 | 0.87 | ATP2A3 | 3.14 | 0.0816 | 0.77 | TRAPPC10 | 2.5 | 0.3694 | 0.48 | ECHDC2 | 2.1 | 0.7754 | 0.65 |
| RICTOR | 4.55 | 0.00069 | 0.79 | ARNTL | 3.14 | 0.0817 | 0.71 | DYM | 2.5 | 0.365 | 0.47 | SIN3A | 2.1 | 0.7832 | 0.59 |
| OSBPL8 | 4.53 | 0.00076 | 0.75 | NUTM2A-AS1 | 3.14 | 0.08 | 0.64 | CASK | 2.5 | 0.3652 | 0.44 | ADCY7 | 2.1 | 0.7816 | 0.48 |
| CXCR4 | 4.52 | 0.00077 | 1.08 | SERINC5 | 3.14 | 0.081 | 0.62 | PTGER4 | 2.49 | 0.3722 | 1.39 | MLLT10 | 2.1 | 0.7754 | 0.4 |
| CD247 | 4.52 | 0.00077 | 0.7 | CNTN5 | 3.12 | 0.086 | 2.81 | ABL2 | 2.49 | 0.3784 | 0.7 | ANKRD28 | 2.1 | 0.7802 | 0.4 |
| 6-Sep | 4.51 | 0.00079 | 0.82 | SYAP1 | 3.12 | 0.0847 | 1.17 | SLC30A9 | 2.49 | 0.3719 | 0.63 | KMT2C | 2.1 | 0.7832 | 0.39 |
| TNRC6B | 4.51 | 0.00081 | 0.56 | RSBN1 | 3.12 | 0.0861 | 1.12 | DYNLT1 | 2.49 | 0.3784 | 0.59 | LINC01550 | 2.09 | 0.7929 | 0.78 |
| KIAA0825 | 4.5 | 0.00084 | 1.28 | GATAD2B | 3.12 | 0.0859 | 0.54 | PDZD8 | 2.49 | 0.3719 | 0.57 | HERC3 | 2.09 | 0.7897 | 0.7 |
| CD6 | 4.5 | 0.00084 | 0.91 | ADARB2 | 3.11 | 0.0883 | 1.53 | ATP9B | 2.49 | 0.3787 | 0.47 | PFKFB3 | 2.09 | 0.7843 | 0.59 |
| MNAT1 | 4.49 | 0.00088 | 0.82 | ITGAL | 3.11 | 0.0883 | 0.56 | BCAS3 | 2.49 | 0.3734 | 0.43 | LMBRD1 | 2.09 | 0.786 | 0.47 |
| GP2 | 4.48 | 0.00088 | 5.5 | TRANK1 | 3.1 | 0.0915 | 0.74 | CUZD1 | 2.48 | 0.3797 | 4.53 | CSGALNACT2 | 2.09 | 0.7929 | 0.45 |
| LCP2 | 4.48 | 0.00088 | 0.81 | EPC2 | 3.1 | 0.0895 | 0.71 | CCDC69 | 2.48 | 0.3855 | 1.04 | TLK1 | 2.09 | 0.7964 | 0.38 |
| FOXO1 | 4.48 | 0.00088 | 0.63 | NLK | 3.1 | 0.0898 | 0.68 | UBE4B | 2.48 | 0.3814 | 0.5 | ERMP1 | 2.08 | 0.8055 | 0.9 |
| ARAP2 | 4.47 | 0.00091 | 0.67 | FGF14 | 3.09 | 0.0921 | 3.01 | ABHD3 | 2.48 | 0.3855 | 0.49 | ITFG2 | 2.08 | 0.798 | 0.77 |
| IFI16 | 4.46 | 0.00097 | 0.75 | UBR1 | 3.09 | 0.0921 | 0.6 | PKN1 | 2.47 | 0.3894 | 0.79 | ANXA4 | 2.08 | 0.8079 | 0.56 |
| PTGDR | 4.45 | 0.00099 | 1.45 | PCM1 | 3.09 | 0.0934 | 0.57 | RFX3-AS1 | 2.47 | 0.3941 | 0.69 | TBCA | 2.08 | 0.8124 | 0.52 |
| RFX3 | 4.45 | 0.00099 | 0.82 | ULK4 | 3.08 | 0.0951 | 0.75 | MICU2 | 2.47 | 0.393 | 0.47 | GALNT11 | 2.08 | 0.7996 | 0.46 |
| CD226 | 4.44 | 0.00105 | 1.13 | LMBR1 | 3.08 | 0.0939 | 0.65 | RABEP1 | 2.47 | 0.3955 | 0.47 | SACS | 2.07 | 0.8157 | 1.21 |
| CHIC2 | 4.44 | 0.00101 | 0.9 | CAST | 3.08 | 0.0958 | 0.61 | ZNF33B | 2.46 | 0.4058 | 0.82 | IL16 | 2.07 | 0.822 | 0.7 |
| ITPR2 | 4.44 | 0.00105 | 0.75 | MAP4K4 | 3.08 | 0.0958 | 0.58 | ATP6V1H | 2.46 | 0.4024 | 0.7 | AGPAT3 | 2.07 | 0.8267 | 0.44 |
| JAKMIP2 | 4.42 | 0.00113 | 1.45 | MCPH1 | 3.07 | 0.0965 | 0.65 | IL10RA | 2.46 | 0.4012 | 0.64 | ARFGEF1 | 2.07 | 0.8225 | 0.43 |
| TNFSF8 | 4.41 | 0.00118 | 1.4 | RNF38 | 3.07 | 0.0967 | 0.56 | PBXIP1 | 2.45 | 0.4062 | 1.07 | SLF1 | 2.07 | 0.8225 | 0.41 |
| CATSPERB | 4.4 | 0.0012 | 1.24 | CLASP2 | 3.06 | 0.1015 | 0.59 | ZFP90 | 2.45 | 0.4101 | 0.93 | RB1CC1 | 2.07 | 0.8267 | 0.39 |
| SIDT1 | 4.4 | 0.00121 | 0.86 | SLC30A8 | 3.05 | 0.1035 | 3.67 | TFCP2 | 2.45 | 0.4115 | 0.51 | CPSF6 | 2.07 | 0.8214 | 0.36 |
| GSK3B | 4.39 | 0.00127 | 0.78 | SLCO3A1 | 3.05 | 0.1041 | 0.85 | AVL9 | 2.45 | 0.4115 | 0.49 | THEM4 | 2.06 | 0.8377 | 1.1 |
| TRG-AS1 | 4.37 | 0.0014 | 0.88 | STK38 | 3.05 | 0.1041 | 0.6 | WDR33 | 2.45 | 0.4101 | 0.48 | TTC33 | 2.06 | 0.8306 | 0.91 |
| SP100 | 4.35 | 0.00149 | 0.65 | FAM49B | 3.05 | 0.1041 | 0.5 | DPYD | 2.45 | 0.4101 | 0.36 | MAPK9 | 2.06 | 0.831 | 0.86 |
| FKBP5 | 4.35 | 0.00147 | 0.48 | CREM | 3.04 | 0.1058 | 1.16 | MTOR | 2.44 | 0.4149 | 0.8 | ZBTB11 | 2.06 | 0.8338 | 0.79 |
| BAZ1A | 4.34 | 0.00155 | 0.84 | ZNF33A | 3.04 | 0.1049 | 0.7 | HNRNPUL1 | 2.44 | 0.4196 | 0.65 | ECHDC1 | 2.06 | 0.831 | 0.66 |
| DOCK11 | 4.34 | 0.00155 | 0.8 | RNGTT | 3.04 | 0.1057 | 0.59 | TIAM2 | 2.44 | 0.4198 | 0.55 | MANBA | 2.06 | 0.8267 | 0.57 |
| PTPN4 | 4.34 | 0.00155 | 0.8 | PPP1R2 | 3.04 | 0.1049 | 0.58 | HERPUD2 | 2.44 | 0.4166 | 0.53 | SLC38A9 | 2.06 | 0.831 | 0.49 |
| 7-Sep | 4.34 | 0.00155 | 0.76 | RBM33 | 3.04 | 0.1046 | 0.57 | NR5A2 | 2.43 | 0.4322 | 2.09 | TRIM56 | 2.06 | 0.8352 | 0.45 |
| CELF2 | 4.34 | 0.00154 | 0.47 | ABHD17B | 3.03 | 0.1083 | 1.17 | HACD4 | 2.43 | 0.4285 | 1.19 | OSBPL9 | 2.06 | 0.8359 | 0.42 |
| ERN1 | 4.29 | 0.0019 | 0.85 | CDKN1B | 3.03 | 0.1091 | 1.17 | ST6GALNAC3 | 2.43 | 0.4322 | 0.95 | PHF3 | 2.06 | 0.8352 | 0.37 |
| RCSD1 | 4.28 | 0.00199 | 0.79 | VPS13D | 3.03 | 0.108 | 0.55 | TRAPPC8 | 2.43 | 0.4295 | 0.56 | DACH1 | 2.05 | 0.8494 | 1.87 |
| CYTH1 | 4.27 | 0.00203 | 0.7 | ODF2L | 3.02 | 0.1098 | 0.61 | SMAP1 | 2.43 | 0.4295 | 0.54 | SNX14 | 2.05 | 0.852 | 0.45 |
| HMBOX1 | 4.24 | 0.00235 | 0.96 | PPHLN1 | 3.02 | 0.1101 | 0.61 | SH3BGRL | 2.43 | 0.4292 | 0.51 | NCOA7 | 2.05 | 0.848 | 0.44 |
| IPCEF1 | 4.22 | 0.0025 | 0.84 | RASGRP1 | 3.02 | 0.112 | 0.53 | TMOD3 | 2.43 | 0.4316 | 0.49 | CAMKMT | 2.05 | 0.8381 | 0.37 |
| SARAF | 4.22 | 0.0025 | 0.77 | NEK1 | 3.01 | 0.1151 | 0.85 | LINC00861 | 2.42 | 0.4341 | 0.71 | CNTRL | 2.05 | 0.8394 | 0.37 |
| NFATC2 | 4.22 | 0.0025 | 0.74 | SLFN5 | 3.01 | 0.1152 | 0.71 | SHPRH | 2.42 | 0.4398 | 0.6 | FAM117A | 2.04 | 0.8575 | 0.83 |
| STX8 | 4.21 | 0.00263 | 0.79 | TMX4 | 3 | 0.1185 | 1.28 | SCMH1 | 2.42 | 0.4336 | 0.53 | MEF2D | 2.04 | 0.8561 | 0.76 |
| EXOC4 | 4.2 | 0.00267 | 0.69 | KATNAL1 | 3 | 0.1163 | 0.84 | PPP4R3B | 2.42 | 0.4333 | 0.49 | POM121 | 2.04 | 0.8607 | 0.45 |
| SACM1L | 4.19 | 0.00279 | 1.07 | GPR174 | 3 | 0.1163 | 0.69 | NEDD9 | 2.42 | 0.4341 | 0.42 | GABPB1 | 2.04 | 0.8597 | 0.43 |
| PPP2R2B | 4.18 | 0.00293 | 0.81 | MAP2K5 | 3 | 0.1163 | 0.58 | ACSF3 | 2.41 | 0.4473 | 1.18 | ATG5 | 2.04 | 0.8607 | 0.43 |
| MAPK1 | 4.16 | 0.00321 | 0.74 | CREBBP | 3 | 0.1163 | 0.54 | INTS7 | 2.41 | 0.4424 | 0.79 | CD99 | 2.04 | 0.8561 | 0.42 |
| RBM6 | 4.16 | 0.00321 | 0.72 | NCK1 | 2.99 | 0.1189 | 0.67 | TCAF2 | 2.41 | 0.4447 | 0.73 | ATXN2 | 2.04 | 0.857 | 0.41 |
| EPB41 | 4.16 | 0.00321 | 0.68 | PHTF2 | 2.97 | 0.1258 | 0.61 | ZKSCAN1 | 2.41 | 0.4452 | 0.73 | ATR | 2.04 | 0.8622 | 0.41 |
| USP24 | 4.15 | 0.00331 | 0.78 | CEP192 | 2.97 | 0.1265 | 0.6 | MIATNB | 2.41 | 0.4424 | 0.66 | CD2 | 2.04 | 0.8538 | 0.37 |
| NAA50 | 4.14 | 0.00337 | 0.87 | ST8SIA4 | 2.97 | 0.1282 | 0.59 | TMEM135 | 2.41 | 0.4443 | 0.55 | ANKIB1 | 2.04 | 0.859 | 0.36 |
| ROCK1 | 4.13 | 0.00355 | 0.75 | BCL2L1 | 2.96 | 0.1288 | 0.88 | ITPKB | 2.41 | 0.4441 | 0.48 | MUC6 | 2.03 | 0.8752 | 3.97 |
| BTBD9 | 4.13 | 0.00350 | 0.72 | ATP11C | 2.96 | 0.1311 | 0.87 | KMT2A | 2.41 | 0.4447 | 0.45 | DPP4 | 2.03 | 0.8735 | 1.96 |
| VTI1A | 4.13 | 0.00359 | 0.68 | ZBTB16 | 2.96 | 0.129 | 0.78 | MBP | 2.41 | 0.4452 | 0.43 | RHOBTB3 | 2.03 | 0.8735 | 1.17 |
| JAZF1 | 4.11 | 0.00387 | 0.71 | UGP2 | 2.96 | 0.1311 | 0.78 | SLC25A13 | 2.4 | 0.4497 | 0.87 | NUPL2 | 2.03 | 0.8752 | 0.93 |
| KLF12 | 4.1 | 0.00389 | 0.65 | SUPT3H | 2.96 | 0.1289 | 0.55 | CHM | 2.4 | 0.4496 | 0.78 | TMEM154 | 2.03 | 0.8752 | 0.84 |
| BPTF | 4.09 | 0.00406 | 0.69 | USP47 | 2.96 | 0.1311 | 0.51 | VPS45 | 2.4 | 0.4497 | 0.67 | MICAL3 | 2.03 | 0.8752 | 0.71 |
| FNBP1 | 4.09 | 0.00403 | 0.66 | SOX6 | 2.95 | 0.1334 | 1.32 | FAM160B1 | 2.4 | 0.4497 | 0.51 | ANKMY1 | 2.03 | 0.8735 | 0.67 |
| HDAC4 | 4.06 | 0.00464 | 0.79 | KLHDC10 | 2.95 | 0.1334 | 0.89 | VPS53 | 2.4 | 0.4497 | 0.5 | INO80 | 2.03 | 0.8732 | 0.51 |
| AFF1 | 4.04 | 0.00496 | 0.72 | SNRK | 2.95 | 0.1316 | 0.61 | BCLAF1 | 2.4 | 0.4533 | 0.49 | PRR14L | 2.03 | 0.8749 | 0.47 |
| FAR2 | 4.03 | 0.00517 | 1.38 | FBXW7 | 2.95 | 0.1315 | 0.53 | PRIM2 | 2.4 | 0.4533 | 0.47 | PPP1R12B | 2.03 | 0.8752 | 0.41 |
| DENND1A | 4.03 | 0.00517 | 0.74 | EPC1 | 2.95 | 0.1327 | 0.53 | BCL2 | 2.4 | 0.4533 | 0.4 | AGPAT4 | 2.02 | 0.8887 | 1.14 |
| RUNX1 | 4.03 | 0.00524 | 0.55 | SLA | 2.95 | 0.1334 | 0.5 | MALAT1 | 2.4 | 0.4533 | 0.07 | ATP10D | 2.02 | 0.8779 | 0.82 |
| NCKAP1L | 4.02 | 0.00526 | 0.92 | C1QTNF3-AMACR | 2.94 | 0.1359 | 0.77 | BCL2L11 | 2.39 | 0.4548 | 1 | BBS9 | 2.02 | 0.8877 | 0.49 |
| STXBP5 | 4.02 | 0.00524 | 0.77 | CHFR | 2.94 | 0.1358 | 0.74 | XPC | 2.39 | 0.4548 | 0.91 | GOLGB1 | 2.02 | 0.8887 | 0.44 |
| NIN | 4.02 | 0.00528 | 0.77 | MGA | 2.94 | 0.1345 | 0.66 | ZFX | 2.39 | 0.4596 | 0.72 | MBTD1 | 2.02 | 0.8787 | 0.42 |
| KIAA1109 | 4.02 | 0.00524 | 0.71 | DIS3L2 | 2.94 | 0.1362 | 0.62 | PPM1K | 2.39 | 0.4533 | 0.71 | RNF216 | 2.02 | 0.878 | 0.4 |
| ZNRF2 | 4.01 | 0.00544 | 0.86 | TULP4 | 2.94 | 0.1362 | 0.58 | ICE1 | 2.39 | 0.4533 | 0.71 | TNKS2 | 2.02 | 0.8799 | 0.4 |
| NPC1 | 4.01 | 0.00544 | 0.84 | AEBP2 | 2.94 | 0.1359 | 0.57 | HEATR5B | 2.39 | 0.4548 | 0.61 | C2CD5 | 2.02 | 0.8825 | 0.38 |
| ATP2B4 | 4.01 | 0.00539 | 0.72 | ALG13 | 2.93 | 0.1396 | 0.63 | AQR | 2.39 | 0.461 | 0.59 | RAB27A | 2.02 | 0.8903 | 0.37 |
| NIPBL | 4.01 | 0.00538 | 0.62 | EHMT1 | 2.92 | 0.1436 | 0.54 | PARP4 | 2.39 | 0.4533 | 0.44 | SMARCA4 | 2.01 | 0.9002 | 0.56 |
| SLC16A7 | 4 | 0.00562 | 0.84 | EP400 | 2.91 | 0.1454 | 0.7 | TBL1X | 2.39 | 0.4551 | 0.44 | SUMF1 | 2.01 | 0.9042 | 0.45 |
| CRACR2A | 3.98 | 0.0062 | 1.24 | EMSY | 2.91 | 0.1479 | 0.66 | BACH2 | 2.38 | 0.4622 | 0.77 | DNAJC5 | 2.01 | 0.8985 | 0.44 |
| IL6ST | 3.97 | 0.00624 | 0.91 | PHYKPL | 2.91 | 0.1476 | 0.62 | SNTB2 | 2.38 | 0.4692 | 0.59 | FTO | 2.01 | 0.9042 | 0.4 |
| PAG1 | 3.97 | 0.00626 | 0.71 | BRD4 | 2.91 | 0.147 | 0.56 | TTC14 | 2.38 | 0.4618 | 0.47 | CHD1 | 2.01 | 0.8973 | 0.38 |
| KAT6A | 3.97 | 0.00628 | 0.65 | MSN | 2.91 | 0.1479 | 0.51 | ACYP2 | 2.38 | 0.4658 | 0.45 | PPP6R2 | 2.01 | 0.9002 | 0.36 |
| P2RY8 | 3.96 | 0.00654 | 1.04 | JAK1 | 2.91 | 0.146 | 0.5 | GYPC | 2.37 | 0.479 | 1.15 | LRRTM4 | 2 | 0.9122 | 2.12 |
| ARHGAP18 | 3.96 | 0.0065 | 0.91 | MAPRE2 | 2.9 | 0.1501 | 0.66 | ARHGEF6 | 2.37 | 0.4789 | 0.56 | CNTN4 | 2 | 0.9122 | 2.11 |
| RAB8B | 3.96 | 0.0065 | 0.8 | RNF111 | 2.9 | 0.151 | 0.62 | NEK7 | 2.37 | 0.4729 | 0.44 | TYW1B | 2 | 0.9085 | 0.75 |
| MPP7 | 3.95 | 0.00663 | 0.93 | PHKB | 2.9 | 0.1502 | 0.56 | CASP8 | 2.37 | 0.4783 | 0.44 | CLOCK | 2 | 0.9151 | 0.66 |
| ZHX2 | 3.94 | 0.00683 | 0.69 | FBXO11 | 2.9 | 0.1493 | 0.51 | CPA1 | 2.36 | 0.4893 | 27.46 | KCTD20 | 2 | 0.9094 | 0.52 |
| INPP4A | 3.94 | 0.0068 | 0.68 | ZEB1 | 2.9 | 0.1493 | 0.5 | VPS9D1 | 2.36 | 0.4807 | 1.18 | SP4 | 2 | 0.9122 | 0.52 |
| ZNF407 | 3.93 | 0.00719 | 0.75 | INPP4B | 2.9 | 0.1492 | 0.48 | MED14 | 2.36 | 0.483 | 0.72 | PARN | 2 | 0.9122 | 0.43 |
| TBC1D5 | 3.93 | 0.00713 | 0.61 | SSH2 | 2.9 | 0.1487 | 0.41 | PPIP5K2 | 2.36 | 0.4807 | 0.64 | ANKRD17 | 2 | 0.9054 | 0.4 |
| SRGN | 3.92 | 0.0075 | 0.83 | CLIC5 | 2.89 | 0.1542 | 0.86 | MSL2 | 2.36 | 0.4871 | 0.55 | NUTM2B-AS1 | 2 | 0.908 | 0.34 |
| AUH | 3.91 | 0.00771 | 0.79 | MAN1A2 | 2.89 | 0.1528 | 0.57 | ROCK2 | 2.36 | 0.4893 | 0.52 | | | | |
| NRDC | 3.9 | 0.00762 | 0.78 | EFR3A | 2.89 | 0.1538 | 0.56 | PPP3CA | 2.36 | 0.4893 | 0.41 | | | | |
| LNPEP | 3.91 | 0.00757 | 0.73 | NABP1 | 2.88 | 0.1588 | 0.83 | TMEM168 | 2.35 | 0.4947 | 0.68 | | | | |
| TRERF1 | 3.86 | 0.00933 | 0.69 | ZMYND11 | 2.88 | 0.1588 | 0.63 | ZBED4 | 2.35 | 0.4894 | 0.61 | | | | |
| MARK2 | 3.85 | 0.00955 | 0.87 | SND1 | 2.88 | 0.1588 | 0.56 | ANKRD13C | 2.35 | 0.4912 | 0.55 | | | | |
| TTC13 | 3.85 | 0.00957 | 0.83 | CHD6 | 2.88 | 0.157 | 0.55 | ARHGAP30 | 2.35 | 0.4926 | 0.54 | | | | |
| ST3GAL1 | 3.85 | 0.00962 | 0.76 | KIAA1468 | 2.87 | 0.1609 | 0.56 | ZBTB1 | 2.35 | 0.4954 | 0.51 | | | | |
| RASA1 | 3.85 | 0.00968 | 0.64 | 9-Sep | 2.8 | 0.1608 | 0.54 | COL4A3BP | 2.35 | 0.4909 | 0.47 | | | | |
| | | | | UBE2K | 2.87 | 0.1588 | 0.52 | TBC1D22A | 2.35 | 0.4943 | 0.43 | | | | |

Abbreviations:
FC = fold change

TABLE 2.4
Fibroblast by treatment

| Gene | Score | Pval | Log₂FC | Gene | Score | Pval | Log₂FC | Gene | Score | Pval | Log₂FC | Gene | Score | Pval | Log₂FC |
|---|---|---|---|---|---|---|---|---|---|---|---|---|---|---|---|
| ZBTB16 | 36.87 | 8.89E−295 | 1.13 | SMOC2 | 4.88 | 6.30E−06 | 0.35 | MPP2 | 2.57 | 3.64E−02 | 0.87 | IGFN1 | 1.55 | 3.30E−01 | 1.56 |
| XIST | 29.93 | 2.83E−194 | 1.24 | GALNT16 | 4.86 | 6.76E−06 | 1.27 | ATXN7 | 2.57 | 3.58E−02 | 0.64 | LGI4 | 1.55 | 3.29E−01 | 1.22 |
| CRISPLD2 | 29.62 | 2.45E−190 | 0.95 | ESRRG | 4.85 | 7.07E−06 | 1.22 | MYOM1 | 2.57 | 3.58E−02 | 0.47 | ETFDH | 1.55 | 3.31E−01 | 0.24 |
| FKBP5 | 29.19 | 6.84E−185 | 0.72 | PAPPA | 4.85 | 7.30E−06 | 0.32 | KANK2 | 2.57 | 3.64E−02 | 0.31 | HBG2 | 1.54 | 3.34E−01 | 3.24 |
| CACNB2 | 26.24 | 1.93E−149 | 1 | HMCN2 | 4.84 | 7.70E−06 | 1.65 | AFF3 | 2.57 | 3.60E−02 | 0.23 | GRIK5 | 1.54 | 3.33E−01 | 1.14 |
| MALAT1 | 25.81 | 1.12E−144 | 0.23 | EFHD1 | 4.81 | 8.60E−06 | 1.12 | ROR1 | 2.57 | 3.64E−02 | 0.16 | CYP4B1 | 1.53 | 3.38E−01 | 2.63 |
| MYH11 | 25.5 | 3.57E−141 | 1.99 | TENM2 | 4.81 | 8.77E−06 | 0.52 | ZNF665 | 2.55 | 3.76E−02 | 0.94 | MTMR7 | 1.53 | 3.39E−01 | 1.33 |
| DDB2 | 24.77 | 2.52E−133 | 1.46 | RBM20 | 4.8 | 9.03E−06 | 1.82 | MT1M | 2.55 | 3.77E−02 | 0.79 | IL10RB | 1.53 | 3.39E−01 | 1.32 |
| ADAMTS9-AS2 | 24.74 | 6.46E−133 | 0.87 | CEL | 4.8 | 9.17E−06 | 1.77 | LAMA5 | 2.55 | 3.75E−02 | 0.52 | MTSS1 | 1.53 | 3.42E−01 | 0.23 |
| PDZRN4 | 23.78 | 7.07E−123 | 2.17 | PLD5 | 4.78 | 9.98E−06 | 1.59 | TARID | 2.54 | 3.84E−02 | 0.52 | AC098617.1 | 1.52 | 3.43E−01 | 0.92 |
| SPARCL1 | 23.75 | 1.41E−122 | 1.04 | CSRP1 | 4.77 | 1.04E−05 | 0.55 | HSDL2 | 2.54 | 3.85E−02 | 0.32 | GADD45G | 1.52 | 3.48E−01 | 0.92 |
| PRUNE2 | 23.58 | 6.96E−121 | 2.41 | CAB39L | 4.73 | 1.27E−05 | 0.45 | FOSL1 | 2.52 | 4.10E−02 | 1.07 | COL4A6 | 1.52 | 3.46E−01 | 0.84 |
| COL6A2 | 22.82 | 3.35E−113 | 0.67 | REV3L | 4.69 | 1.56E−05 | 0.15 | CHST3 | 2.52 | 4.09E−02 | 0.51 | SOD3 | 1.52 | 3.48E−01 | 0.68 |
| LPP | 22.48 | 5.91E−110 | 0.27 | WNK2 | 4.66 | 1.75E−05 | 1.6 | KIAA1671 | 2.52 | 4.15E−02 | 0.44 | MX1 | 1.52 | 3.48E−01 | 0.51 |
| SORBS1 | 21.47 | 2.68E−100 | 0.75 | AGBL4 | 4.6 | 2.37E−05 | 0.79 | ARHGAP22 | 2.51 | 4.20E−02 | 0.31 | MTMR1 | 1.52 | 3.45E−01 | 0.24 |
| ANKS1B | 21.16 | 1.74E−97 | 1.25 | FHL5 | 4.58 | 2.61E−05 | 0.82 | CELA3A | 2.5 | 4.29E−02 | 3.42 | KCNJ3 | 1.51 | 3.51E−01 | 1.39 |
| RBPMS | 19.73 | 7.54E−85 | 0.32 | IFI44L | 4.58 | 2.63E−05 | 0.66 | DPP10 | 2.49 | 4.38E−02 | 1.27 | NTNG1 | 1.51 | 3.51E−01 | 0.61 |
| SYNPO2 | 19.53 | 3.42E−83 | 0.83 | FGFR1 | 4.57 | 2.73E−05 | 0.27 | COL4A4 | 2.49 | 4.41E−02 | 0.29 | PRKACA | 1.51 | 3.54E−01 | 0.38 |
| DMD | 19.44 | 2.19E−82 | 0.7 | ALOX15B | 4.56 | 2.89E−05 | 1.52 | SOBP | 2.48 | 4.50E−02 | 0.25 | PTP4A1 | 1.51 | 3.54E−01 | 0.29 |
| NR4A1 | 19.38 | 6.76E−82 | 1.3 | CUZD1 | 4.53 | 3.27E−05 | 2.71 | NDE1 | 2.47 | 4.62E−02 | 0.43 | GGPS1 | 1.5 | 3.60E−01 | 2.31 |
| ELN | 19.1 | 1.38E−79 | 0.79 | SMCR5 | 4.51 | 3.52E−05 | 1.15 | SLC33A1 | 2.46 | 4.73E−02 | 2.65 | USP2 | 1.5 | 3.56E−01 | 1.16 |
| RBMS3 | 18.61 | 1.38E−75 | 0.49 | GPHN | 4.5 | 3.75E−05 | 0.12 | LSAMP-AS1 | 2.46 | 4.81E−02 | 0.73 | PPP1R14A | 1.5 | 3.56E−01 | 0.63 |
| IGFBP4 | 18.22 | 1.91E−72 | 0.9 | ADAMTS15 | 4.4 | 5.75E−05 | 1.25 | SNHG19 | 2.45 | 4.94E−02 | 0.96 | DCLK1 | 1.5 | 3.55E−01 | 0.21 |
| TACC1 | 18.12 | 1.17E−71 | 0.6 | LONRF1 | 4.4 | 5.92E−05 | 0.36 | SYNE3 | 2.43 | 5.14E−02 | 0.5 | LTBP1 | 1.5 | 3.57E−01 | −0.02 |
| SAMD4A | 17.88 | 8.53E−70 | 0.69 | ADHFE1 | 4.35 | 7.23E−05 | 1.12 | PACSIN2 | 2.43 | 5.17E−02 | 0.21 | KBTBD12 | 1.49 | 3.62E−01 | 0.84 |
| PDK4 | 17.62 | 7.80E−68 | 1.54 | IL6ST | 4.35 | 7.10E−05 | 0.27 | NALT1 | 2.42 | 5.23E−02 | 1.55 | ESYT1 | 1.49 | 3.62E−01 | 0.6 |
| DES | 17.42 | 2.49E−66 | 4.1 | DEPDC5 | 4.34 | 7.65E−05 | 3.01 | VAV3 | 2.42 | 5.26E−02 | 0.59 | UNC79 | 1.49 | 3.65E−01 | 0.5 |
| TGFBR3 | 16.79 | 1.26E−61 | 1.12 | SLFNL1 | 4.34 | 7.65E−05 | 1.46 | CDH19 | 2.41 | 5.43E−02 | 1.86 | SYNP0 | 1.49 | 3.64E−01 | 0.37 |
| PDE4D | 16.57 | 4.55E−60 | 0.56 | A2M | 4.34 | 7.68E−05 | 0.44 | RPS20 | 2.41 | 5.44E−02 | 0.44 | IGF2 | 1.48 | 3.70E−01 | 6.31 |
| NEGR1 | 15.88 | 2.98E−55 | 0.58 | MTIE | 4.33 | 7.89E−05 | 0.93 | ELAVL1 | 2.41 | 5.37E−02 | 0.38 | C4orf36 | 1.48 | 3.68E−01 | 2.06 |
| DGKG | 15.61 | 2.23E−53 | 2.2 | AC159540.1 | 4.32 | 8.18E−05 | 2.88 | SH3BP5 | 2.41 | 5.44E−02 | 0.38 | PCSK1N | 1.48 | 3.68E−01 | 1.71 |
| ACTG2 | 15.42 | 4.01E−52 | 3.01 | CPED1 | 4.32 | 8.06E−05 | 0.28 | NFATC1 | 2.39 | 5.72E−02 | 0.4 | LINC01242 | 1.48 | 3.71E−01 | 1.16 |
| MT2A | 15.4 | 5.68E−52 | 0.87 | PNLIP | 4.3 | 9.02E−05 | 4.85 | SCO2 | 2.38 | 5.80E−02 | 3.8 | FRAS1 | 1.48 | 3.68E−01 | 0.32 |
| FAM129A | 15.35 | 1.19E−51 | 1.36 | PDE7B | 4.29 | 9.17E−05 | 0.48 | GPRC5C | 2.38 | 5.92E−02 | 1.12 | STOX2 | 1.48 | 3.70E−01 | 0.27 |
| SAMHD1 | 15.31 | 2.10E−51 | 0.86 | RAMP1 | 4.28 | 9.91E−05 | 0.56 | LINC01290 | 2.38 | 5.84E−02 | 0.85 | ITGA9 | 1.48 | 3.67E−01 | 0.21 |
| LMOD1 | 15.02 | 1.75E−49 | 1.08 | RPS19 | 4.28 | 9.57E−05 | 0.48 | RGL3 | 2.38 | 5.81E−02 | 0.69 | CKB | 1.47 | 3.77E−01 | 0.58 |
| SLC7A2 | 14.99 | 2.79E−49 | 1.83 | FDXR | 4.27 | 1.03E−04 | 1.71 | PLEKHA4 | 2.36 | 6.16E−02 | 0.4 | BAX | 1.47 | 3.76E−01 | 0.4 |
| ADAMTS1 | 14.29 | 7.05E−45 | 1.39 | SLC30A8 | 4.26 | 1.08E−04 | 1.54 | SPATA6 | 2.36 | 6.18E−02 | 0.23 | ARHGAP11B | 1.46 | 3.83E−01 | 2.76 |
| KCNAB1 | 14.28 | 7.83E−45 | 2.13 | NR3C2 | 4.18 | 1.51E−04 | 0.36 | AMT | 2.35 | 6.24E−02 | 2.65 | FAM189A2 | 1.46 | 3.84E−01 | 1.65 |
| PID1 | 14.27 | 1.02E−44 | 0.67 | GPX3 | 4.16 | 1.60E−04 | 1.14 | COL4A5 | 2.34 | 6.47E−02 | 0.26 | GNA14-AS1 | 1.46 | 3.82E−01 | 1.03 |
| FHL1 | 14.2 | 2.44E−44 | 1.46 | CACNA1C | 4.16 | 1.60E−04 | 0.05 | ASB2 | 2.33 | 6.54E−02 | 2.69 | NAPA | 1.46 | 3.80E−01 | 0.34 |
| MYOCD | 14.17 | 3.98E−44 | 2.57 | IRF2BP2 | 4.14 | 1.79E−04 | 0.49 | GDF15 | 2.33 | 6.63E−02 | 1.56 | SEMA6A | 1.46 | 3.81E−01 | 0.33 |
| SLC4A4 | 14 | 4.27E−43 | 1.11 | LRRFIP1 | 4.14 | 1.77E−04 | 0.22 | EPHX2 | 2.32 | 6.82E−02 | 0.83 | CTRC | 1.45 | 3.85E−01 | 2.65 |
| COL21A1 | 13.98 | 5.76E−43 | 1.19 | RANBP3L | 4.13 | 1.84E−04 | 1.59 | GPM6A | 2.31 | 6.89E−02 | 1.54 | AC009264.1 | 1.45 | 3.85E−01 | 1.56 |
| PER1 | 13.89 | 2.04E−42 | 0.87 | LIFR | 4.12 | 1.88E−04 | 0.44 | DPT | 2.31 | 6.92E−02 | 1.14 | GPR20 | 1.45 | 3.86E−01 | 1.27 |
| SLC19A2 | 13.73 | 1.79E−41 | 0.88 | HMGCLL1 | 4.11 | 2.01E−04 | 0.98 | CHST7 | 2.3 | 7.08E−02 | 0.71 | FXYD2 | 1.44 | 3.97E−01 | 3.54 |
| ELL2 | 13.67 | 4.05E−41 | 0.66 | GARNL3 | 4.11 | 2.03E−04 | 0.51 | CDC42EP4 | 2.28 | 7.54E−02 | 0.94 | TXLNG | 1.44 | 3.97E−01 | 0.23 |
| AKAP6 | 13.52 | 2.98E−40 | 2.12 | LPAR1 | 4.09 | 2.16E−04 | 0.31 | ZFP36 | 2.28 | 7.42E−02 | 0.36 | AC012593.1 | 1.43 | 4.03E−01 | 2.04 |
| MEG3 | 13.45 | 7.60E−40 | 0.77 | OSMR | 4.08 | 2.27E−04 | 0.25 | ENOSF1 | 2.28 | 7.40E−02 | 0.22 | FSHR | 1.43 | 4.02E−01 | 1.92 |
| COL14A1 | 13.4 | 1.38E−39 | 0.86 | NR5A2 | 4.06 | 2.45E−04 | 0.93 | NR4A2 | 2.27 | 7.71E−02 | 0.56 | SCARA5 | 1.43 | 4.03E−01 | 1.11 |
| LDB3 | 13.32 | 4.18E−39 | 2.36 | MAMLD1 | 4.06 | 2.43E−04 | 0.63 | COMMD7 | 2.26 | 7.87E−02 | 3.58 | PPM1B | 1.43 | 4.02E−01 | 0.16 |
| MDM2 | 13.31 | 4.66E−39 | 0.72 | RGL1 | 4.05 | 2.53E−04 | 0.4 | GALNT15 | 2.26 | 7.84E−02 | 1.03 | C1orf21 | 1.43 | 4.02E−01 | 0.13 |
| MIR34AHG | 13.27 | 8.73E−39 | 0.82 | ERO1B | 4.04 | 2.60E−04 | 0.51 | FBXO42 | 2.26 | 7.87E−02 | 0.17 | BCAM | 1.42 | 4.07E−01 | 0.96 |
| DLG2 | 13.27 | 8.60E−39 | 0.34 | PTGDS | 4.03 | 2.78E−04 | 2.42 | LSP1 | 2.25 | 8.02E−02 | 1.25 | CLN8 | 1.41 | 4.12E−01 | 2.61 |
| DTNA | 13.13 | 4.89E−38 | 1.1 | CD59 | 4.01 | 3.00E−04 | 1.29 | TRIM5 | 2.25 | 7.98E−02 | 0.45 | PART1 | 1.41 | 4.10E−01 | 2.31 |
| PTPRG | 13.12 | 6.06E−38 | 0.07 | SERPINE1 | 4.01 | 3.01E−04 | 0.28 | MPP7 | 2.24 | 8.26E−02 | 0.6 | CLN5 | 1.41 | 4.15E−01 | 1.9 |
| NID1 | 12.71 | 1.08E−35 | 0.67 | RIMS2 | 4 | 3.07E−04 | 0.93 | MX2 | 2.24 | 8.09E−02 | 0.46 | MFAP4 | 1.41 | 4.12E−01 | 0.63 |
| TMTC1 | 12.6 | 4.73E−35 | 0.87 | MBP | 3.99 | 3.27E−04 | 0.44 | SOX13 | 2.24 | 8.27E−02 | 0.34 | SST | 1.4 | 4.22E−01 | 2.86 |
| RYR2 | 12.36 | 9.24E−34 | 0.73 | CTNND2 | 3.98 | 3.40E−04 | 0.54 | RPL34 | 2.23 | 8.46E−02 | 0.54 | LINC01320 | 1.4 | 4.20E−01 | 1.5 |
| ABI3BP | 12.35 | 1.01E−33 | 0.79 | PROS1 | 3.98 | 3.36E−04 | 0.49 | DGKB | 2.23 | 8.33E−02 | 0.44 | DND1 | 1.4 | 4.18E−01 | 1.02 |
| CELF2 | 12.35 | 9.86E−34 | 0.68 | GPC3 | 3.97 | 3.46E−04 | 0.83 | TBCEL | 2.22 | 8.65E−02 | 2.93 | DLGAP2 | 1.4 | 4.18E−01 | 0.87 |
| BCL6 | 12.33 | 1.30E−33 | 0.78 | AC026202.3 | 3.92 | 4.22E−04 | 1.85 | CNTN5 | 2.22 | 8.51E−02 | 0.63 | GCNT2 | 1.4 | 4.22E−01 | 0.86 |
| MTHFD1L | 12.29 | 2.12E−33 | 0.46 | LINC00685 | 3.92 | 4.23E−04 | 0.66 | CLDN11 | 2.21 | 8.69E−02 | 0.97 | PIR | 1.4 | 4.22E−01 | 0.44 |
| CDKNIA | 12.23 | 4.39E−33 | 1.73 | RPL31 | 3.92 | 4.24E−04 | 0.59 | LONRF3 | 2.21 | 8.71E−02 | 0.7 | ACER2 | 1.4 | 4.17E−01 | 0.36 |
| KCNMA1 | 12.16 | 1.03E−32 | 0.98 | MTUS1 | 3.92 | 4.26E−04 | 0.27 | PGF | 2.21 | 8.73E−02 | 0.63 | SOCS2 | 1.4 | 4.19E−01 | 0.3 |
| GP2 | 12.05 | 3.60E−32 | 4.98 | SLC38A1 | 3.91 | 4.49E−04 | 0.58 | SPDYE16 | 2.2 | 8.95E−02 | 0.91 | MATN2 | 1.4 | 4.20E−01 | 0.28 |
| ITGA5 | 11.96 | 1.05E−31 | 0.96 | FBLN5 | 3.91 | 4.38E−04 | 0.35 | SCARA3 | 2.2 | 9.00E−02 | 0.57 | RFX6 | 1.39 | 4.23E−01 | 1.95 |
| ESYT2 | 11.93 | 1.62E−31 | 0.39 | TNS1 | 3.9 | 4.59E−04 | 0.08 | PPARGC1B | 2.18 | 9.30E−02 | 0.63 | NTRK1 | 1.39 | 4.26E−01 | 1.41 |
| NFIB | 11.9 | 2.16E−31 | 0.42 | RYR3 | 3.89 | 4.71E−04 | 1.52 | ARHGEF1OL | 2.16 | 9.81E−02 | 0.28 | FAM124A | 1.38 | 4.33E−01 | 0.54 |
| NAV2 | 11.84 | 4.40E−31 | 0.59 | FMN2 | 3.88 | 5.06E−04 | 1.06 | RBPJL | 2.15 | 1.01E−01 | 4.06 | CSF1 | 1.38 | 4.32E−01 | 0.44 |
| MAMDC2 | 11.79 | 7.91E−31 | 0.97 | ESR1 | 3.88 | 5.04E−04 | 0.5 | RASD1 | 2.15 | 9.98E−02 | 1.38 | G6PC2 | 1.37 | 4.42E−01 | 2.07 |
| COL4A2 | 11.71 | 2.00E−30 | 0.34 | SVIL | 3.87 | 5.22E−04 | 0.06 | HLF | 2.15 | 9.98E−02 | 0.82 | PPP2R2B | 1.37 | 4.36E−01 | 0.41 |
| SPEG | 11.67 | 3.31E−30 | 2.15 | ARPC1A | 3.86 | 5.29E−04 | 3.29 | COLEC12 | 2.15 | 1.01E−01 | 0.38 | C3 | 1.37 | 4.37E−01 | 0.2 |
| STAT3 | 11.54 | 1.42E−29 | 0.35 | CHRDL2 | 3.85 | 5.64E−04 | 3.18 | SERTAD2 | 2.15 | 1.00E−01 | 0.3 | CAPN3 | 1.36 | 4.44E−01 | 3.34 |
| PTCHD4 | 11.34 | 1.49E−28 | 2.03 | CACNA1A | 3.84 | 5.69E−04 | 0.72 | ATP7A | 2.14 | 1.02E−01 | 2.51 | NCAM1 | 1.36 | 4.45E−01 | 1.08 |
| FOXO3 | 11.3 | 2.30E−28 | 0.36 | MAP3K14 | 3.84 | 5.90E−04 | 0.48 | LINC00886 | 2.14 | 1.04E−01 | 0.56 | PRKCB | 1.36 | 4.44E−01 | 0.74 |
| LONRF2 | 11.17 | 9.44E−28 | 1.46 | IFI6 | 3.83 | 5.97E−04 | 1 | EBF2 | 2.14 | 1.03E−01 | 0.3 | POLR2L | 1.36 | 4.48E−01 | 0.66 |
| ECHDC2 | 10.94 | 1.22E−26 | 0.57 | MMP19 | 3.83 | 6.07E−04 | 0.49 | TM7SF3 | 2.14 | 1.02E−01 | 0.26 | MKNK2 | 1.36 | 4.48E−01 | 0.37 |
| FGF14 | 10.89 | 2.13E−26 | 1.15 | PKHD1 | 3.82 | 6.15E−04 | 0.63 | KCNK3 | 2.13 | 1.05E−01 | 2.29 | PIDD1 | 1.35 | 4.50E−01 | 0.46 |
| TAGLN | 10.83 | 3.89E−26 | 0.64 | FAM13C | 3.82 | 6.28E−04 | 0.5 | TRAF3IP2-AS1 | 2.13 | 1.05E−01 | 0.18 | NUP153 | 1.35 | 4.50E−01 | 0.17 |
| COL6A1 | 10.64 | 2.85E−25 | 0.49 | FAM49A | 3.8 | 6.73E−04 | 0.51 | RERGL | 2.11 | 1.09E−01 | 1.17 | SNRPN | 1.34 | 4.62E−01 | 1.6 |
| ZMAT3 | 10.61 | 4.13E−25 | 0.86 | CPA2 | 3.79 | 7.04E−04 | 3.5 | DYSF | 2.11 | 1.10E−01 | 1.1 | ADRA1B | 1.34 | 4.57E−01 | 1.45 |
| CFTR | 10.42 | 3.14E−24 | 0.99 | CRLF1 | 3.79 | 7.08E−04 | 1.23 | TMEM132D | 2.11 | 1.10E−01 | 0.96 | UNC13C | 1.34 | 4.57E−01 | 1.34 |
| ADAMTSL3 | 10.34 | 6.57E−24 | 0.92 | C1QTNF1 | 3.79 | 6.97E−04 | 0.78 | EDNRB | 2.11 | 1.11E−01 | 0.88 | MYT1L | 1.34 | 4.61E−01 | 1.33 |
| TNXB | 10.28 | 1.29E−23 | 1.17 | PNLIPRP1 | 3.77 | 7.64E−04 | 4.45 | SGSM1 | 2.11 | 1.09E−01 | 0.81 | PHYHD1 | 1.34 | 4.59E−01 | 0.55 |
| TACC2 | 10.14 | 5.35E−23 | 1.09 | ZNF483 | 3.76 | 7.84E−04 | 0.48 | LMO3 | 2.1 | 1.12E−01 | 0.3 | ZCCHC2 | 1.34 | 4.57E−01 | 0.28 |
| NTRK3 | 10.05 | 1.28E−22 | 1.51 | IFITM3 | 3.75 | 8.07E−04 | 0.62 | TRIM9 | 2.09 | 1.14E−01 | 0.94 | GABPB2 | 1.34 | 4.61E−01 | 0.19 |
| GCG | 9.98 | 2.61E−22 | 2.44 | KCNB1 | 3.73 | 8.81E−04 | 1.73 | NUPR1 | 2.09 | 1.14E−01 | 0.51 | ADARB2 | 1.33 | 4.68E−01 | 0.89 |
| FILIP1 | 9.97 | 2.85E−22 | 0.82 | LRRTM4 | 3.73 | 8.71E−04 | 0.94 | BCOR | 2.09 | 1.14E−01 | 0.48 | KLKB1 | 1.33 | 4.68E−01 | 0.82 |
| ARHGAP10 | 9.89 | 6.24E−22 | 0.3 | ARHGAP26 | 3.73 | 8.66E−04 | 0.25 | LRP1B | 2.08 | 1.18E−01 | 0.55 | LRRC2 | 1.33 | 4.67E−01 | 0.76 |
| KCNMB2-AS1 | 9.84 | 1.04E−21 | 1.33 | PDK3 | 3.71 | 9.58E−04 | 0.4 | LINC00670 | 2.07 | 1.21E−01 | 1.71 | KCTD12 | 1.33 | 4.63E−01 | 0.33 |
| HIF3A | 9.76 | 2.23E−21 | 0.73 | SLC22A3 | 3.7 | 9.88E−04 | 0.68 | LAMA3 | 2.07 | 1.20E−01 | 0.29 | CHRM3-AS2 | 1.32 | 4.72E−01 | 3.26 |
| MYL9 | 9.75 | 2.45E−21 | 0.61 | CLMN | 3.7 | 9.90E−04 | 0.24 | STEAP4 | 2.06 | 1.23E−01 | 0.81 | EML5 | 1.32 | 4.72E−01 | 0.69 |
| DPP6 | 9.72 | 3.27E−21 | 1.22 | AMY2A | 3.69 | 1.04E−03 | 7.6 | CFAP161 | 2.06 | 1.24E−01 | 0.76 | TM4SF1-AS1 | 1.3 | 4.90E−01 | 1.52 |
| NLGN1 | 9.58 | 1.25E−20 | 0.92 | NPNT | 3.69 | 1.02E−03 | 0.33 | CSNKIE | 2.06 | 1.24E−01 | 0.53 | SRL | 1.3 | 4.90E−01 | 1.21 |
| PLPP3 | 9.47 | 3.57E−20 | 1.06 | RBFOX3 | 3.68 | 1.05E−03 | 2.54 | KIAA1324L | 2.06 | 1.22E−01 | 0.3 | KIF12 | 1.3 | 4.87E−01 | 1.01 |
| SYNE1 | 9.47 | 3.51E−20 | 0.27 | CPE | 3.68 | 1.08E−03 | 0.41 | SLC16A7 | 2.06 | 1.22E−01 | 0.29 | TBCE | 1.3 | 4.90E−01 | 0.71 |
| ST3GAL3 | 9.46 | 3.79E−20 | 2.56 | CXCL12 | 3.67 | 1.11E−03 | 0.8 | ITGA10 | 2.05 | 1.27E−01 | 0.48 | GASIRR | 1.3 | 4.85E−01 | 0.41 |
| AXL | 9.3 | 1.73E−19 | 0.49 | NTN1 | 3.66 | 1.14E−03 | 0.87 | TCEAL4 | 2.05 | 1.26E−01 | 0.39 | HOMER2 | 1.3 | 4.91E−01 | 0.39 |
| HSPA4L | 9.29 | 1.92E−19 | 1.65 | RBPMS2 | 3.65 | 1.20E−03 | 1.18 | SEMA4A | 2.04 | 1.28E−01 | 1.04 | RGS2 | 1.29 | 4.94E−01 | 0.45 |
| BCL2 | 9.14 | 7.68E−19 | 0.64 | ABCA8 | 3.64 | 1.22E−03 | 0.43 | GAS6-AS1 | 2.04 | 1.29E−01 | 0.71 | GNAO1 | 1.29 | 4.95E−01 | 0.34 |
| SLIT3 | 9.14 | 7.55E−19 | 0.47 | ARHGAP29 | 3.64 | 1.21E−03 | 0.37 | KLF15 | 2.03 | 1.30E−01 | 0.73 | GCSH | 1.28 | 5.04E−01 | 1.83 |
| USP39 | 9.12 | 8.91E−19 | 0.92 | CNR1 | 3.63 | 1.29E−03 | 1.77 | NTN4 | 2.03 | 1.32E−01 | 0.42 | CCDC141 | 1.28 | 5.02E−01 | 0.94 |
| SYNM | 9.05 | 1.65E−18 | 2.71 | AC100830.3 | 3.62 | 1.34E−03 | 3.8 | HIPK2 | 2.03 | 1.30E−01 | 0.25 | GLIPR1L2 | 1.28 | 5.02E−01 | 0.83 |
| SORT1 | 9.05 | 1.66E−18 | 0.87 | NFIL3 | 3.61 | 1.37E−03 | 0.75 | PDE8B | 2.02 | 1.35E−01 | 1.12 | C2 | 1.28 | 4.99E−01 | 0.74 |
| CSPG4 | 8.96 | 3.71E−18 | 1.84 | KLHL13 | 3.6 | 1.45E−03 | 0.49 | DSCAM | 2.02 | 1.35E−01 | 1.09 | RSPO3 | 1.28 | 4.99E−01 | 0.53 |
| FBLN1 | 8.91 | 6.15E−18 | 0.49 | TRIM22 | 3.59 | 1.46E−03 | 0.35 | GYPC | 2.02 | 1.35E−01 | 0.51 | SH3RF3 | 1.28 | 5.03E−01 | 0.02 |
| SIK3 | 8.9 | 6.45E−18 | 0.21 | TRPM3 | 3.58 | 1.55E−03 | 0.83 | CBX7 | 2.02 | 1.33E−01 | 0.39 | KCNJ16 | 1.27 | 5.06E−01 | 0.92 |
| MAP2 | 8.83 | 1.26E−17 | 0.79 | ARL17B | 3.58 | 1.53E−03 | 0.41 | SLC9A9 | 2.02 | 1.35E−01 | 0.18 | RPS6KA6 | 1.27 | 5.10E−01 | 0.41 |
| PLCE1 | 8.82 | 1.31E−17 | 0.53 | PRSS1 | 3.57 | 1.60E−03 | 4.69 | CRHR1 | 2.01 | 1.38E−01 | 2.69 | INPP1 | 1.27 | 5.06E−01 | 0.28 |
| EGR1 | 8.78 | 1.81E−17 | 0.61 | MEF2C-AS1 | 3.57 | 1.58E−03 | 0.43 | HSPB6 | 2.01 | 1.37E−01 | 1.49 | RFX3-AS1 | 1.27 | 5.05E−01 | 0.21 |
| JPH2 | 8.71 | 3.48E−17 | 2.07 | SUSD5 | 3.55 | 1.71E−03 | 1.38 | RIMBP2 | 2.01 | 1.37E−01 | 0.86 | PKNOX2 | 1.27 | 5.07E−01 | 0.21 |
| NNMT | 8.55 | 1.36E−16 | 0.48 | ANGPTL1 | 3.55 | 1.70E−03 | 0.87 | COL15A1 | 2.01 | 1.36E−01 | 0.3 | KCNH1 | 1.26 | 5.18E−01 | 1.16 |
| CTNNA3 | 8.54 | 1.45E−16 | 0.86 | ZNF804B | 3.53 | 1.81E−03 | 2.56 | IMMP2L | 2 | 1.39E−01 | −0.07 | PTX3 | 1.26 | 5.17E−01 | 0.96 |
| GABPB1-AS1 | 8.54 | 1.46E−16 | 0.63 | ADARB1 | 3.53 | 1.86E−03 | 0.4 | PTCHD1-AS | 1.99 | 1.44E−01 | 1.04 | ST18 | 1.26 | 5.15E−01 | 0.74 |
| DYNC1I1 | 8.46 | 2.88E−16 | 0.7 | PARL | 3.52 | 1.94E−03 | 2.77 | HFM1 | 1.99 | 1.44E−01 | 0.72 | SLC3A1 | 1.26 | 5.14E−01 | 0.65 |
| RFX2 | 8.44 | 3.32E−16 | 0.67 | FGF2 | 3.52 | 1.87E−03 | 0.47 | OSBP2 | 1.99 | 1.44E−01 | 0.68 | CASP9 | 1.26 | 5.12E−01 | 0.46 |
| SOD2 | 8.37 | 5.96E−16 | 2.23 | CYR61 | 3.52 | 1.87E−03 | 0.26 | SPRY2 | 1.99 | 1.42E−01 | 0.67 | C8orf37-AS1 | 1.25 | 5.24E−01 | 0.47 |
| NOVA1 | 8.27 | 1.44E−15 | 0.91 | ACADL | 3.51 | 1.95E−03 | 1.36 | CHD4 | 1.99 | 1.44E−01 | 0.63 | ID4 | 1.25 | 5.27E−01 | 0.35 |
| TMOD1 | 8.18 | 2.84E−15 | 1.71 | RAB30-AS1 | 3.51 | 1.99E−03 | 0.42 | CLPS | 1.98 | 1.45E−01 | 5.77 | NEURLIB | 1.25 | 5.23E−01 | 0.17 |
| CNN1 | 8.14 | 4.25E−15 | 1.12 | TSPYL2 | 3.49 | 2.08E−03 | 0.38 | PNCK | 1.98 | 1.47E−01 | 3.07 | B3GAT2 | 1.24 | 5.29E−01 | 0.83 |
| JADE1 | 8.09 | 6.03E−15 | 0.6 | FAM85B | 3.48 | 2.18E−03 | 0.34 | ANKRD29 | 1.98 | 1.45E−01 | 0.69 | ZNF98 | 1.24 | 5.33E−01 | 0.36 |
| ASTN2 | 8 | 1.22E−14 | 0.72 | TNFAIP8L3 | 3.47 | 2.30E−03 | 1.3 | CD34 | 1.98 | 1.45E−01 | 0.68 | SIDT2 | 1.24 | 5.32E−01 | 0.34 |
| EGFR | 7.99 | 1.38E−14 | 0.36 | LGR6 | 3.46 | 2.33E−03 | 2.02 | UNC80 | 1.97 | 1.49E−01 | 1.3 | C9orf106 | 1.23 | 5.37E−01 | 1.95 |
| KCTD7 | 7.88 | 3.35E−14 | 2.96 | RGS5 | 3.46 | 2.39E−03 | 0.69 | ALPK3 | 1.96 | 1.53E−01 | 0.79 | CES1 | 1.23 | 5.42E−01 | 0.9 |
| NTRK2 | 7.83 | 4.87E−14 | 1.12 | CNTNAP2 | 3.45 | 2.42E−03 | 0.81 | TNFRSF10D | 1.96 | 1.53E−01 | 0.58 | MRGPRF | 1.23 | 5.38E−01 | 0.69 |
| LINC01482 | 7.82 | 5.19E−14 | 0.9 | CDH13 | 3.45 | 2.42E−03 | 0.56 | CCDC3 | 1.96 | 1.51E−01 | 0.37 | BLOC1S2 | 1.23 | 5.37E−01 | 0.29 |
| SETBP1 | 7.82 | 5.25E−14 | 0.27 | HSPB1 | 3.45 | 2.39E−03 | 0.44 | OSR1 | 1.95 | 1.56E−01 | 1.04 | SHANK2 | 1.23 | 5.38E−01 | 0.28 |
| ADCY5 | 7.8 | 5.83E−14 | 1.07 | CSDC2 | 3.44 | 2.48E−03 | 1.32 | AKAP12 | 1.95 | 1.53E−01 | 0.18 | PLA2G4C | 1.23 | 5.40E−01 | 0.26 |
| ARHGEF26 | 7.7 | 1.30E−13 | 0.6 | REG1A | 3.43 | 2.60E−03 | 2.35 | PCGF5 | 1.95 | 1.55E−01 | 0.18 | NFIX | 1.23 | 5.39E−01 | 0.18 |
| SVEP1 | 7.7 | 1.34E−13 | 0.52 | FRMD3 | 3.43 | 2.61E−03 | 0.5 | SERPINE2 | 1.94 | 1.59E−01 | 0.5 | MED17 | 1.22 | 5.42E−01 | 1.16 |
| RCAN2 | 7.68 | 1.47E−13 | 0.75 | REG3A | 3.41 | 2.78E−03 | 3.78 | ADGRL3 | 1.94 | 1.59E−01 | 0.39 | ANO5 | 1.22 | 5.48E−01 | 1.16 |
| CPB1 | 7.65 | 1.92E−13 | 3.92 | C6 | 3.34 | 3.52E−03 | 1.03 | KCNMB1 | 1.94 | 1.58E−01 | 0.32 | MLXIPL | 1.22 | 5.43E−01 | 1 |
| PAK3 | 7.61 | 2.70E−13 | 0.7 | RPLP2 | 3.34 | 3.56E−03 | 0.51 | OR2A1-AS1 | 1.93 | 1.62E−01 | 3.03 | WARS2-IT1 | 1.22 | 5.43E−01 | 0.8 |
| RALYL | 7.49 | 6.61E−13 | 1.61 | IQCJ-SCHIP1 | 3.34 | 3.50E−03 | 0.39 | RPL13A | 1.93 | 1.60E−01 | 0.32 | CACNA2D3 | 1.22 | 5.49E−01 | 0.37 |
| ZBTB7C | 7.49 | 6.61E−13 | 0.78 | ADAMTS8 | 3.33 | 3.72E−03 | 1.9 | AGFG2 | 1.92 | 1.63E−01 | 0.9 | MIR217HG | 1.21 | 5.53E−01 | 2.58 |
| CHRM3 | 7.48 | 6.67E−13 | 1.54 | MTHFR | 3.33 | 3.71E−03 | 0.51 | DNAH11 | 1.91 | 1.70E−01 | 0.77 | MRAP2 | 1.21 | 5.55E−01 | 1.12 |
| NFASC | 7.4 | 1.21E−12 | 0.66 | CHST9 | 3.32 | 3.74E−03 | 1.37 | TCEA3 | 1.91 | 1.69E−01 | 0.47 | NRG2 | 1.21 | 5.57E−01 | 0.67 |
| NFIA | 7.39 | 1.29E−12 | 0.19 | LINC01322 | 3.32 | 3.74E−03 | 0.96 | CSRNP1 | 1.9 | 1.71E−01 | 0.55 | ENSA | 1.21 | 5.56E−01 | 0.18 |
| TTR | 7.36 | 1.63E−12 | 3.04 | RNASE10 | 3.3 | 4.05E−03 | 1.67 | AKAP1 | 1.9 | 1.71E−01 | 0.48 | TC2N | 1.21 | 5.57E−01 | 0.18 |
| ZFHX3 | 7.3 | 2.58E−12 | 0.27 | ADGRB3 | 3.29 | 4.14E−03 | 0.41 | AGGF1 | 1.89 | 1.73E−01 | 2.07 | PCP4 | 1.2 | 5.61E−01 | 1.56 |
| LRP4 | 7.26 | 3.57E−12 | 1.03 | PPM1L | 3.28 | 4.39E−03 | 0.43 | NTF3 | 1.89 | 1.76E−01 | 0.86 | OTOGL | 1.2 | 5.62E−01 | 1.16 |
| SSBP2 | 7.24 | 3.89E−12 | 0.2 | SLC8A1 | 3.27 | 4.54E−03 | 0.3 | TSIX | 1.89 | 1.74E−01 | 0.67 | GPC1 | 1.2 | 5.61E−01 | 0.48 |
| C7 | 7.19 | 5.72E−12 | 0.49 | PHF20 | 3.25 | 4.80E−03 | 0.24 | LAMC3 | 1.89 | 1.73E−01 | 0.66 | DAAM1 | 1.2 | 5.62E−01 | 0.14 |
| RERG | 7.19 | 5.62E−12 | 0.43 | RPS17 | 3.24 | 4.99E−03 | 1.45 | TRHDE | 1.89 | 1.74E−01 | 0.62 | MLIP | 1.19 | 5.73E−01 | 0.95 |
| LINC01088 | 7.16 | 7.03E−12 | 0.44 | ATP1A1 | 3.21 | 5.51E−03 | 0.3 | LINC01515 | 1.89 | 1.76E−01 | 0.52 | DTD1 | 1.19 | 5.72E−01 | 0.55 |
| ASPA | 7.15 | 7.42E−12 | 1.4 | RTN1 | 3.2 | 5.56E−03 | 1.23 | CCDC107 | 1.89 | 1.75E−01 | 0.43 | RNF4 | 1.19 | 5.66E−01 | 0.28 |
| THRB | 7.14 | 8.01E−12 | 0.45 | GTF2IRD2B | 3.19 | 5.78E−03 | 0.48 | FAM184B | 1.88 | 1.77E−01 | 0.56 | GRK5 | 1.19 | 5.67E−01 | 0.19 |
| ITIH5 | 7.13 | 8.52E−12 | 0.86 | 3-Mar | 3.18 | 6.04E−03 | 0.36 | SPATA22 | 1.86 | 1.84E−01 | 0.84 | VPS33B | 1.18 | 5.80E−01 | 3.06 |
| PER2 | 7.12 | 9.40E−12 | 0.76 | GMPR | 3.17 | 6.28E−03 | 1.49 | CELA3B | 1.85 | 1.90E−01 | 2.63 | RMND5B | 1.18 | 5.76E−01 | 0.32 |
| EDA2R | 7.08 | 1.27E−11 | 1.56 | ZHX2 | 3.17 | 6.16E−03 | 0.18 | Cllorf54 | 1.85 | 1.90E−01 | 2.45 | RRM2B | 1.18 | 5.81E−01 | 0.29 |
| AASS | 7.03 | 1.83E−11 | 0.54 | SCN3A | 3.16 | 6.32E−03 | 1.5 | EXOC3-AS1 | 1.85 | 1.88E−01 | 1.33 | RBM24 | 1.17 | 5.85E−01 | 3.06 |
| NRXN1 | 6.96 | 2.82E−11 | 1.62 | DENND4A | 3.16 | 6.32E−03 | 0.2 | MYEF2 | 1.85 | 1.90E−01 | 0.59 | LIMD2 | 1.17 | 5.88E−01 | 2.47 |
| MCAM | 6.96 | 2.91E−11 | 1.17 | EPB42 | 3.14 | 6.83E−03 | 2.19 | FOSB | 1.85 | 1.89E−01 | 0.45 | ALDH1L1 | 1.17 | 5.88E−01 | 1.21 |
| HPSE2 | 6.94 | 3.40E−11 | 1.96 | STARD9 | 3.14 | 6.73E−03 | 0.3 | PITPNM2 | 1.85 | 1.91E−01 | 0.35 | LIFR-AS1 | 1.17 | 5.84E−01 | 0.8 |
| PGM5 | 6.9 | 4.46E−11 | 1.38 | DAAM2 | 3.13 | 7.06E−03 | 0.33 | GAS6 | 1.85 | 1.90E−01 | 0.25 | PDGFRL | 1.17 | 5.83E−01 | 0.44 |
| TBC1D1 | 6.88 | 4.92E−11 | 0.42 | WISP2 | 3.12 | 7.34E−03 | 2.5 | GLUL | 1.85 | 1.89E−01 | 0.24 | NT5E | 1.17 | 5.86E−01 | 0.4 |
| BTG2 | 6.87 | 5.28E−11 | 1.29 | CDON | 3.11 | 7.53E−03 | 0.65 | OGN | 1.83 | 1.98E−01 | 0.52 | WARS2 | 1.17 | 5.85E−01 | 0.19 |
| ADAM33 | 6.84 | 6.66E−11 | 0.8 | MAPK4 | 3.1 | 7.67E−03 | 2.27 | PHKA1 | 1.83 | 1.96E−01 | 0.51 | DACT3 | 1.16 | 5.93E−01 | 0.7 |
| CTRB1 | 6.83 | 6.97E−11 | 4.48 | AC002066.1 | 3.1 | 7.74E−03 | 0.72 | SPRY1 | 1.83 | 1.97E−01 | 0.24 | STON2 | 1.16 | 5.94E−01 | 0.22 |
| ADAMTS3 | 6.83 | 7.15E−11 | 0.93 | SESN2 | 3.09 | 7.88E−03 | 0.93 | CLIP1 | 1.81 | 2.04E−01 | 0.17 | UHRF1BP1L | 1.16 | 5.89E−01 | 0.18 |
| ABLIM1 | 6.8 | 8.74E−11 | 0.62 | PEAR1 | 3.08 | 8.23E−03 | 0.6 | PRSS2 | 1.8 | 2.08E−01 | 1.83 | ACTN1 | 1.16 | 5.92E−01 | 0.03 |
| AOX1 | 6.78 | 9.76E−11 | 0.9 | GNG7 | 3.07 | 8.52E−03 | 0.61 | MYRIP | 1.8 | 2.09E−01 | 0.58 | DHRS12 | 1.15 | 5.99E−01 | 3.74 |
| ZNF544 | 6.76 | 1.15E−10 | 3.21 | YBX3 | 3.07 | 8.54E−03 | 0.22 | KCND3 | 1.8 | 2.08E−01 | 0.38 | FAM107A | 1.15 | 6.04E−01 | 1.7 |
| ZNF331 | 6.74 | 1.27E−10 | 0.86 | CHN2 | 3.04 | 9.30E−03 | 2.95 | FXN | 1.79 | 2.11E−01 | 3.68 | SGCA | 1.15 | 6.00E−01 | 1.69 |
| IL6R | 6.73 | 1.35E−10 | 1.26 | ADH1B | 3.04 | 9.22E−03 | 0.67 | AP002884.2 | 1.79 | 2.15E−01 | 2.9 | ZNF350-AS1 | 1.15 | 6.01E−01 | 0.77 |
| PARM1 | 6.72 | 1.52E−10 | 1.03 | ATP6V0A1 | 3.02 | 9.96E−03 | 0.27 | STAC2 | 1.79 | 2.11E−01 | 2.37 | GFRA1 | 1.15 | 6.00E−01 | 0.43 |
| CRY1 | 6.71 | 1.58E−10 | 0.54 | TENM1 | 3.01 | 1.02E−02 | 1.01 | GKAP1 | 1.79 | 2.12E−01 | 0.56 | RNF122 | 1.15 | 6.00E−01 | 0.39 |
| ZC3H11A | 6.6 | 3.38E−10 | 3.09 | TLE2 | 3.01 | 1.03E−02 | 0.63 | PER3 | 1.79 | 2.13E−01 | 0.31 | LIG1 | 1.15 | 6.02E−01 | 0.37 |
| KALRN | 6.6 | 3.40E−10 | 0.25 | PPM1D | 3.01 | 1.01E−02 | 0.39 | ATOH8 | 1.78 | 2.18E−01 | 1.76 | TBX18 | 1.15 | 5.99E−01 | 0.31 |
| ITGA7 | 6.56 | 4.35E−10 | 1.34 | NGF | 2.99 | 1.08E−02 | 1.1 | CCDC39 | 1.78 | 2.17E−01 | 1.01 | DDX24 | 1.15 | 5.99E−01 | 0.2 |
| ITGA8 | 6.56 | 4.25E−10 | 1.18 | RAB20 | 2.99 | 1.10E−02 | 0.96 | WDR63 | 1.78 | 2.16E−01 | 0.95 | ABCF2 | 1.14 | 6.05E−01 | 3.15 |
| ARHGEF10 | 6.5 | 6.30E−10 | 2.78 | PPARGCIA | 2.99 | 1.09E−02 | 0.91 | SCML1 | 1.78 | 2.19E−01 | 0.27 | FN3K | 1.14 | 6.06E−01 | 0.7 |
| PRDM16 | 6.42 | 1.05E−09 | 1.31 | NFATC2 | 2.96 | 1.19E−02 | 0.43 | ZFAND5 | 1.78 | 2.19E−01 | 0.21 | PLA2G5 | 1.14 | 6.08E−01 | 0.67 |
| HS6ST3 | 6.37 | 1.51E−09 | 1.85 | MFSD14A | 2.95 | 1.23E−02 | 1.94 | OLFML3 | 1.77 | 2.22E−01 | 0.83 | EIF2B5 | 1.14 | 6.06E−01 | 0.44 |
| ZNF385D | 6.31 | 2.15E−09 | 0.89 | PTN | 2.95 | 1.25E−02 | 1.37 | SLC9B1 | 1.77 | 2.23E−01 | 0.61 | TTN | 1.14 | 6.06E−01 | 0.32 |
| MUC6 | 6.3 | 2.29E−09 | 2.42 | KLF9 | 2.95 | 1.25E−02 | 0.25 | MCF2L | 1.76 | 2.25E−01 | 0.74 | SPTLC3 | 1.14 | 6.07E−01 | 0.3 |
| RAD50 | 6.29 | 2.43E−09 | 2.32 | ATP1A2 | 2.94 | 1.26E−02 | 1.99 | MTERF2 | 1.76 | 2.24E−01 | 0.46 | SLC47A1 | 1.13 | 6.16E−01 | 0.92 |
| EBF1 | 6.27 | 2.78E−09 | 0.27 | GALNT18 | 2.94 | 1.25E−02 | 0.55 | ACTB | 1.76 | 2.25E−01 | 0.15 | VWF | 1.13 | 6.14E−01 | 0.55 |
| NR4A3 | 6.25 | 3.11E−09 | 1.57 | CAMK2G | 2.94 | 1.25E−02 | 0.53 | KIAA1324 | 1.75 | 2.30E−01 | 1.01 | CALN1 | 1.13 | 6.19E−01 | 0.49 |
| ACOT11 | 6.23 | 3.45E−09 | 1.31 | HSPB7 | 2.93 | 1.29E−02 | 2.22 | HSPB8 | 1.75 | 2.32E−01 | 0.96 | IER5 | 1.13 | 6.19E−01 | 0.38 |
| ARHGAP6 | 6.22 | 3.83E−09 | 0.46 | ADRA1A | 2.93 | 1.32E−02 | 2.07 | SLC25A18 | 1.75 | 2.31E−01 | 0.63 | EEF2K | 1.13 | 6.17E−01 | 0.21 |
| MEDAG | 6.21 | 4.07E−09 | 1.01 | TPM2 | 2.93 | 1.31E−02 | 0.38 | PCSK2 | 1.74 | 2.33E−01 | 1.45 | COL4A3 | 1.13 | 6.18E−01 | 0.15 |
| PPP1R12B | 6.16 | 5.50E−09 | 0.29 | SLC35A3 | 2.92 | 1.33E−02 | 2.97 | FGF10 | 1.74 | 2.36E−01 | 0.5 | MROH7-TTC4 | 1.12 | 6.27E−01 | 2.23 |
| ZFP36L1 | 6.12 | 7.02E−09 | 0.24 | TIGAR | 2.92 | 1.35E−02 | 0.52 | CYP4X1 | 1.73 | 2.38E−01 | 1.53 | SPOCK3 | 1.12 | 6.27E−01 | 0.92 |
| ZNF37A | 6.07 | 9.49E−09 | 2.91 | CTNNA2 | 2.91 | 1.39E−02 | 1.12 | ABCC8 | 1.73 | 2.37E−01 | 1.43 | SLC7A5 | 1.12 | 6.21E−01 | 0.55 |
| COL4A1 | 6.06 | 1.02E−08 | 0.24 | SNTG1 | 2.9 | 1.43E−02 | 1.19 | ST8SIA1 | 1.73 | 2.40E−01 | 0.62 | SCGN | 1.11 | 6.36E−01 | 1.74 |
| RIC3 | 6.04 | 1.09E−08 | 2.44 | VIT | 2.88 | 1.53E−02 | 1.83 | MERTK | 1.73 | 2.38E−01 | 0.27 | NMNAT2 | 1.11 | 6.32E−01 | 0.7 |
| RGMA | 6.04 | 1.15E−08 | 1.56 | AC072062.1 | 2.86 | 1.63E−02 | 2.3 | RAB26 | 1.72 | 2.41E−01 | 1.07 | MYO3B | 1.11 | 6.36E−01 | 0.46 |
| P2RX1 | 6.01 | 1.33E−08 | 3.38 | TOB2 | 2.86 | 1.62E−02 | 0.54 | CADM2 | 1.72 | 2.44E−01 | 0.76 | CPT1A | 1.11 | 6.36E−01 | 0.24 |
| SRPX | 5.96 | 1.78E−08 | 0.53 | SLC16A12 | 2.84 | 1.69E−02 | 1.5 | OTUD7A | 1.71 | 2.47E−01 | 0.26 | FAM184A | 1.1 | 6.42E−01 | 0.37 |
| HACD1 | 5.95 | 1.97E−08 | 1.74 | GLIPR1 | 2.83 | 1.74E−02 | 0.59 | DSEL | 1.7 | 2.53E−01 | 0.4 | ST6GALNAC6 | 1.09 | 6.53E−01 | 3.19 |
| TNC | 5.9 | 2.57E−08 | 0.78 | SBSPON | 2.82 | 1.79E−02 | 1.67 | GABRB1 | 1.69 | 2.55E−01 | 2.04 | MIR7-3HG | 1.09 | 6.54E−01 | 2.22 |
| CLU | 5.85 | 3.53E−08 | 1.79 | KIF5C | 2.82 | 1.82E−02 | 0.71 | ISYNA1 | 1.69 | 2.56E−01 | 0.5 | AVPR1A | 1.09 | 6.53E−01 | 1.12 |
| CPA1 | 5.81 | 4.37E−08 | 5.11 | IFNAR2 | 2.81 | 1.87E−02 | 2.97 | CEBPD | 1.68 | 2.63E−01 | 0.24 | NGFR | 1.09 | 6.47E−01 | 0.84 |
| DSCAML1 | 5.78 | 5.18E−08 | 0.91 | CD9 | 2.81 | 1.87E−02 | 0.54 | SNHG25 | 1.67 | 2.68E−01 | 0.56 | ADGRV1 | 1.09 | 6.54E−01 | 0.69 |
| MYLK | 5.76 | 5.97E−08 | 0.46 | DST | 2.8 | 1.94E−02 | −0.1 | PRDM11 | 1.67 | 2.65E−01 | 0.31 | DCDC2 | 1.09 | 6.48E−01 | 0.46 |
| ITPR1 | 5.74 | 6.66E−08 | 0.35 | KCTD8 | 2.79 | 1.96E−02 | 1.76 | SERPING1 | 1.67 | 2.64E−01 | 0.27 | KCNH2 | 1.08 | 6.61E−01 | 2.17 |
| LINC01481 | 5.73 | 6.73E−08 | 2.97 | RPS16 | 2.79 | 1.94E−02 | 0.57 | LINC01197 | 1.66 | 2.73E−01 | 0.39 | AVPI1 | 1.08 | 6.58E−01 | 1.26 |
| CHRM2 | 5.7 | 8.40E−08 | 2.58 | KCNB2 | 2.78 | 2.05E−02 | 1.2 | PPP1R9A | 1.66 | 2.73E−01 | 0.27 | ESAM | 1.08 | 6.56E−01 | 0.79 |
| EPAS1 | 5.7 | 8.34E−08 | 0.49 | ZNF540 | 2.78 | 2.03E−02 | 0.51 | LINC01099 | 1.65 | 2.77E−01 | 1.79 | COCH | 1.08 | 6.61E−01 | 0.77 |
| USP53 | 5.7 | 8.37E−08 | 0.4 | TJP2 | 2.77 | 2.09E−02 | 2.39 | C21orf62-AS1 | 1.65 | 2.73E−01 | 0.43 | LINC00222 | 1.08 | 6.59E−01 | 0.56 |
| RGS6 | 5.68 | 9.20E−08 | 1.16 | PCDH11X | 2.77 | 2.11E−02 | 1.42 | PWRN1 | 1.64 | 2.80E−01 | 1.54 | CITED2 | 1.08 | 6.62E−01 | 0.46 |
| RPS6KA5 | 5.64 | 1.17E−07 | 0.68 | IGF1 | 2.77 | 2.11E−02 | 0.52 | DNAJC27-AS1 | 1.64 | 2.80E−01 | 0.62 | OR2L13 | 1.07 | 6.70E−01 | 3.21 |
| TTLL7 | 5.63 | 1.24E−07 | 0.64 | CASQ2 | 2.76 | 2.13E−02 | 3.01 | BDNF-AS | 1.64 | 2.80E−01 | 0.33 | SLC16A9 | 1.07 | 6.66E−01 | 0.65 |
| REEP1 | 5.59 | 1.51E−07 | 2.15 | CPXM2 | 2.76 | 2.16E−02 | 0.42 | GJA5 | 1.62 | 2.89E−01 | 1.56 | FRZB | 1.07 | 6.67E−01 | 0.31 |
| GFOD1 | 5.55 | 1.93E−07 | 0.74 | LINC00989 | 2.75 | 2.19E−02 | 1.38 | CERS3-AS1 | 1.62 | 2.92E−01 | 1.23 | ATG9A | 1.06 | 6.77E−01 | 1.69 |
| SPATA18 | 5.54 | 2.06E−07 | 1.02 | GAREM1 | 2.74 | 2.24E−02 | 0.28 | CACNB4 | 1.62 | 2.94E−01 | 0.37 | GATM | 1.06 | 6.76E−01 | 0.64 |
| LMNA | 5.53 | 2.16E−07 | 0.4 | MAOA | 2.73 | 2.31E−02 | 0.78 | SPANXA2-OT1 | 1.61 | 3.00E−01 | 1.23 | ADAMTS5 | 1.06 | 6.78E−01 | 0.64 |
| ENPEP | 5.52 | 2.30E−07 | 0.81 | AC058791.1 | 2.7 | 2.52E−02 | 0.9 | SNCA | 1.61 | 2.95E−01 | 1.2 | CORO2B | 1.06 | 6.73E−01 | 0.24 |
| GLDN | 5.44 | 3.58E−07 | 2.02 | NOL4 | 2.68 | 2.66E−02 | 1.44 | SHC4 | 1.61 | 2.96E−01 | 0.76 | SDCBP2-AS1 | 1.06 | 6.74E−01 | 0.22 |
| STOM | 5.43 | 3.59E−07 | 0.59 | NFIC | 2.68 | 2.65E−02 | 0.16 | FGFR2 | 1.61 | 2.95E−01 | 0.59 | EMP2 | 1.06 | 6.78E−01 | 0.2 |
| VWCE | 5.41 | 4.09E−07 | 1.77 | LRRTM3 | 2.67 | 2.76E−02 | 1.11 | RPS28 | 1.6 | 3.03E−01 | 0.73 | PI16 | 1.05 | 6.82E−01 | 1.84 |
| IGSF9B | 5.35 | 5.74E−07 | 1.93 | ZBTB21 | 2.67 | 2.74E−02 | 0.56 | RADIL | 1.6 | 3.01E−01 | 0.68 | CORO6 | 1.05 | 6.85E−01 | 0.56 |
| METTL7A | 5.34 | 5.94E−07 | 0.78 | GNA14 | 2.66 | 2.84E−02 | 0.55 | RELN | 1.59 | 3.06E−01 | 0.69 | CEACAM19 | 1.05 | 6.86E−01 | 0.53 |
| ADIRF | 5.28 | 8.38E−07 | 1.35 | LINC01376 | 2.66 | 2.84E−02 | 0.39 | FGF10-AS1 | 1.59 | 3.10E−01 | 0.67 | ADSL | 1.04 | 6.89E−01 | 2.56 |
| B3GALNT2 | 5.28 | 8.38E−07 | 0.52 | ANGPT1 | 2.66 | 2.80E−02 | 0.35 | SIPR3 | 1.59 | 3.07E−01 | 0.52 | ADPRHL1 | 1.04 | 6.88E−01 | 0.85 |
| DUSP1 | 5.28 | 8.11E−07 | 0.36 | SPDYA | 2.65 | 2.91E−02 | 0.76 | TWIST2 | 1.59 | 3.07E−01 | 0.42 | MUC5B | 1.04 | 6.92E−01 | 0.57 |
| DENND3 | 5.26 | 8.98E−07 | 0.95 | DDIT4 | 2.65 | 2.89E−02 | 0.38 | PRKCE | 1.59 | 3.06E−01 | 0.13 | AGTR1 | 1.04 | 6.91E−01 | 0.36 |
| CNTNAP3B | 5.25 | 9.47E−07 | 1.3 | VIPR2 | 2.63 | 3.05E−02 | 0.81 | GRID2 | 1.58 | 3.13E−01 | 0.85 | CAV1 | 1.04 | 6.90E−01 | 0.26 |
| XPC | 5.25 | 9.58E−07 | 0.45 | TMEM38B | 2.63 | 3.05E−02 | 0.46 | POU6F2 | 1.58 | 3.11E−01 | 0.61 | CYB5R3 | 1.04 | 6.91E−01 | 0.22 |
| ECE1 | 5.25 | 9.70E−07 | 0.39 | ATF6B | 2.63 | 3.11E−02 | 0.45 | SLC44A2 | 1.58 | 3.12E−01 | 0.38 | PAH | 1.03 | 6.99E−01 | 2.39 |
| DPY19L2 | 5.13 | 1.76E−06 | 0.84 | POLH | 2.63 | 3.04E−02 | 0.41 | APOD | 1.58 | 3.15E−01 | 0.24 | DNAJB11 | 1.03 | 6.99E−01 | 0.63 |
| NT5DC3 | 5.13 | 1.77E−06 | 0.69 | WFDC1 | 2.62 | 3.14E−02 | 1.25 | RNF157 | 1.57 | 3.19E−01 | 0.87 | CAMK1 | 1.03 | 6.99E−01 | 0.43 |
| CREB5 | 5.08 | 2.34E−06 | 0.62 | ERBB4 | 2.62 | 3.14E−02 | 0.39 | GPM6B | 1.57 | 3.18E−01 | 0.53 | CTNNAL1 | 1.03 | 6.95E−01 | 0.23 |
| PPP1CB | 5.04 | 2.75E−06 | 0.38 | ZCWPW2 | 2.61 | 3.26E−02 | 2.24 | CIRL | 1.57 | 3.18E−01 | 0.21 | FBXO16 | 1.02 | 7.10E−01 | 0.6 |
| CMKLR1 | 5.02 | 3.16E−06 | 1.26 | BBC3 | 2.59 | 3.39E−02 | 1 | SMYD1 | 1.56 | 3.22E−01 | 5.68 | TESK2 | 1.02 | 7.06E−01 | 0.31 |
| NDUFAF6 | 5.02 | 3.07E−06 | 0.5 | CADM1 | 2.59 | 3.39E−02 | 0.54 | KDM4A | 1.56 | 3.23E−01 | 3.11 | IFIT1 | 1.01 | 7.12E−01 | 1.23 |
| ALDOA | 4.97 | 3.94E−06 | 3.45 | APOLD1 | 2.58 | 3.48E−02 | 0.75 | PCDH15 | 1.56 | 3.21E−01 | 0.9 | NPR1 | 1.01 | 7.17E−01 | 1.19 |
| PLCXD3 | 4.94 | 4.76E−06 | 0.99 | ST6GALNAC3 | 2.58 | 3.52E−02 | 0.7 | EDA | 1.56 | 3.21E−01 | 0.33 | ASIC2 | 1.01 | 7.14E−01 | 0.78 |
| RGN | 4.88 | 6.13E−06 | 1.15 | UBC | 2.58 | 3.50E−02 | 0.25 | SMTN | 1.56 | 3.22E−01 | 0.3 | DCC | 1.01 | 7.20E−01 | 0.53 |
| SSBP3 | 4.88 | 6.33E−06 | 0.38 | SEMA3E | 2.57 | 3.60E−02 | 1.23 | TIMP3 | 1.56 | 3.25E−01 | 0.16 | PLEKHO1 | 1.01 | 7.19E−01 | 0.38 |
| | | | | | | | | | | | | PTPRO | 1 | 7.29E−01 | 0.87 |
| | | | | | | | | | | | | STAG3 | 1 | 7.22E−01 | 0.46 |

Abbreviations: FC = fold change

TABLE 2.5
Macrophage by treatment

| Gene | Score | Pval | Log₂FC | Gene | Score | Pval | Log₂FC | Gene | Score | Pval | Log₂FC | Gene | Score | Pval | Log₂FC |
|---|---|---|---|---|---|---|---|---|---|---|---|---|---|---|---|
| XIST | 22.25 | 2.69E−105 | 2.15 | AP1B1 | 4.47 | 2.81E−04 | 0.34 | PATL2 | 3.12 | 3.53E−02 | 0.93 | GNA13 | 2.42 | 2.13E−01 | 0.18 |
| ZBTB16 | 17.95 | 1.11E−68 | 1.43 | PKHD1 | 4.46 | 2.94E−04 | 1.23 | TPST1 | 3.12 | 3.60E−02 | 0.59 | MAN2A1 | 2.42 | 2.15E−01 | 0.18 |
| CELF2 | 17 | 1.40E−61 | 0.62 | EMILIN2 | 4.46 | 2.93E−04 | 0.42 | SORBS2 | 3.12 | 3.61E−02 | 0.54 | P2RY14 | 2.41 | 2.16E−01 | 0.93 |
| GP2 | 16.04 | 8.76E−55 | 8.08 | CHKA | 4.46 | 2.92E−04 | 0.29 | RASSF1 | 3.12 | 3.61E−02 | 0.4 | PPM1F | 2.41 | 2.17E−01 | 0.4 |
| AOAH | 15.93 | 4.88E−54 | 1.07 | DIRC3 | 4.45 | 3.02E−04 | 0.52 | ATXN10 | 3.11 | 3.66E−02 | 0.25 | CEP63 | 2.41 | 2.19E−01 | 0.35 |
| SLC4A4 | 15.19 | 4.29E−49 | 2.56 | ATF6 | 4.43 | 3.37E−04 | 0.33 | PLD3 | 3.1 | 3.77E−02 | 0.33 | PGS1 | 2.41 | 2.19E−01 | 0.3 |
| ARHGAP24 | 15 | 6.16E−48 | 0.99 | NRXN1 | 4.41 | 3.60E−04 | 1.93 | DPH6 | 3.1 | 3.83E−02 | 0.32 | NDE1 | 2.41 | 2.16E−01 | 0.28 |
| MEF2C | 14.85 | 5.75E−47 | 1.07 | CNTRL | 4.39 | 4.00E−04 | 0.33 | RB1 | 3.1 | 3.81E−02 | 0.21 | BTG1 | 2.41 | 2.18E−01 | 0.21 |
| LDLRAD4 | 14.82 | 8.30E−47 | 0.56 | GPAT3 | 4.37 | 4.42E−04 | 1.36 | NR4A1 | 3.09 | 3.95E−02 | 0.79 | NAA15 | 2.41 | 2.18E−01 | 0.19 |
| MS4A6A | 13.83 | 1.01E−40 | 0.9 | NHLRC2 | 4.37 | 4.42E−04 | 0.51 | FAM193A | 3.08 | 3.99E−02 | 0.24 | RHOQ | 2.41 | 2.17E−01 | 0.18 |
| STARD13 | 13.78 | 2.09E−40 | 0.91 | S100Z | 4.34 | 4.81E−04 | 0.67 | GALNT2 | 3.08 | 4.00E−02 | 0.24 | GIMAP8 | 2.4 | 2.22E−01 | 0.62 |
| SRGAP2 | 13.78 | 2.09E−40 | 0.87 | TYROBP | 4.33 | 5.15E−04 | 0.66 | TTN | 3.06 | 4.29E−02 | 1.37 | SUFU | 2.4 | 2.22E−01 | 0.2 |
| PID1 | 13.74 | 3.25E−40 | 1.73 | RALYL | 4.32 | 5.37E−04 | 1.81 | ZSWIM7 | 3.06 | 4.30E−02 | 0.45 | UNC79 | 2.39 | 2.27E−01 | 1.48 |
| USP53 | 13.38 | 4.14E−38 | 1.16 | SIL1 | 4.32 | 5.37E−04 | 0.38 | ZNF277 | 3.06 | 4.31E−02 | 0.3 | NSUN6 | 2.39 | 2.27E−01 | 0.37 |
| FKBP5 | 13.12 | 1.24E−36 | 0.51 | ELF2 | 4.32 | 5.29E−04 | 0.26 | TGFBR2 | 3.06 | 4.20E−02 | 0.22 | MAVS | 2.39 | 2.27E−01 | 0.29 |
| TBXAS1 | 13.11 | 1.43E−36 | 0.51 | PIK3CD | 4.31 | 5.65E−04 | 0.52 | ZNF532 | 3.05 | 4.40E−02 | 0.36 | ST3GAL5 | 2.39 | 2.28E−01 | 0.28 |
| DISC1 | 12.76 | 1.33E−34 | 0.99 | HIP1 | 4.31 | 5.58E−04 | 0.43 | ATG16L2 | 3.05 | 4.41E−02 | 0.32 | TIAM2 | 2.39 | 2.30E−01 | 0.23 |
| GAS7 | 12.5 | 3.32E−33 | 0.95 | CPA2 | 4.3 | 5.80E−04 | 4.55 | RBPJL | 3.04 | 4.49E−02 | 6.59 | PI4KA | 2.39 | 2.26E−01 | 0.15 |
| PDE3B | 12.43 | 8.09E−33 | 0.91 | LOXL3 | 4.3 | 5.86E−04 | 0.74 | ITPKB | 3.04 | 4.54E−02 | 0.37 | PRKAG2 | 2.39 | 2.29E−01 | 0.06 |
| MAML3 | 11.99 | 1.57E−30 | 0.65 | AL592183.1 | 4.3 | 5.79E−04 | 0.64 | ELL | 3.04 | 4.56E−02 | 0.3 | KLHL18 | 2.38 | 2.33E−01 | 0.38 |
| CSGALNACT1 | 11.98 | 1.87E−30 | 1.2 | JPX | 4.3 | 5.81E−04 | 0.35 | PECAM1 | 3.03 | 4.65E−02 | 0.24 | ATF7IP2 | 2.38 | 2.31E−01 | 0.24 |
| CFTR | 11.91 | 4.17E−30 | 1.85 | CHN2 | 4.29 | 5.93E−04 | 2.13 | FAM117B | 3.02 | 4.78E−02 | 0.35 | ZXDC | 2.38 | 2.31E−01 | 0.19 |
| CD163 | 11.88 | 5.81E−30 | 0.79 | TBC1D5 | 4.27 | 6.53E−04 | 0.14 | NETO2 | 3 | 5.04E−02 | 0.68 | SNX30 | 2.38 | 2.34E−01 | 0.19 |
| SLC9A9 | 11.71 | 4.08E−29 | 1.03 | FAR2 | 4.25 | 7.19E−04 | 0.36 | PLA2G4A | 3 | 5.04E−02 | 0.39 | TTLL11 | 2.37 | 2.40E−01 | 0.75 |
| PIK3R5 | 11.68 | 5.76E−29 | 0.62 | ARMCX4 | 4.24 | 7.49E−04 | 0.67 | SUSD6 | 3 | 5.04E−02 | 0.19 | C5 | 2.37 | 2.38E−01 | 0.61 |
| ELMO1 | 11.41 | 1.27E−27 | 0.43 | CD74 | 4.23 | 7.69E−04 | 0.31 | ZBTB46 | 2.99 | 5.18E−02 | 0.43 | ACCS | 2.37 | 2.40E−01 | 0.59 |
| NAIP | 11.04 | 8.10E−26 | 0.72 | ZFX | 4.22 | 7.98E−04 | 0.47 | RGS1 | 2.99 | 5.24E−02 | 0.22 | ARHGEF1 | 2.37 | 2.40E−01 | 0.32 |
| CPVL | 10.96 | 1.80E−25 | 0.91 | RTN1 | 4.22 | 7.86E−04 | 0.4 | AREG | 2.98 | 5.32E−02 | 0.92 | FGD3 | 2.37 | 2.38E−01 | 0.25 |
| DGKD | 10.51 | 2.25E−23 | 1.1 | TMCC3 | 4.21 | 8.35E−04 | 0.38 | CNTLN | 2.98 | 5.36E−02 | 0.27 | ATG4C | 2.37 | 2.38E−01 | 0.21 |
| ACSL1 | 10.51 | 2.18E−23 | 0.6 | COMMD10 | 4.21 | 8.35E−04 | 0.31 | IL17RA | 2.98 | 5.36E−02 | 0.26 | XPO6 | 2.37 | 2.38E−01 | 0.21 |
| SRGAP2B | 10.43 | 5.03E−23 | 0.84 | HEATR3 | 4.2 | 8.69E−04 | 0.53 | KLHDC1 | 2.97 | 5.51E−02 | 0.47 | ZNF131 | 2.37 | 2.38E−01 | 0.2 |
| PTPRE | 10.36 | 9.82E−23 | 0.85 | TM6SF1 | 4.2 | 8.73E−04 | 0.36 | NISCH | 2.97 | 5.47E−02 | 0.43 | PLAGL1 | 2.37 | 2.40E−01 | 0.2 |
| STAB1 | 10.24 | 3.46E−22 | 1.08 | SORL1 | 4.2 | 8.78E−04 | 0.34 | ENSA | 2.97 | 5.47E−02 | 0.3 | MPEG1 | 2.36 | 2.43E−01 | 1.26 |
| FCHSD2 | 10.13 | 1.08E−21 | 0.46 | RASSF2 | 4.19 | 8.90E−04 | 0.48 | FAM208B | 2.97 | 5.44E−02 | 0.24 | LST1 | 2.36 | 2.42E−01 | 0.48 |
| SRGAP1 | 10.12 | 1.08E−21 | 0.61 | EMB | 4.16 | 1.00E−03 | 0.65 | PTCHD4 | 2.96 | 5.67E−02 | 3.74 | RNPC3 | 2.36 | 2.43E−01 | 0.23 |
| SPIDR | 10.12 | 1.08E−21 | 0.38 | ANKMY1 | 4.16 | 1.03E−03 | 0.48 | SASH3 | 2.96 | 5.59E−02 | 0.79 | SPI1 | 2.36 | 2.42E−01 | 0.22 |
| HDAC9 | 10.01 | 3.25E−21 | 0.94 | CCM2 | 4.15 | 1.04E−03 | 0.39 | PRMT2 | 2.96 | 5.65E−02 | 0.23 | PPP6R2 | 2.36 | 2.43E−01 | 0.18 |
| SLA | 9.8 | 2.62E−20 | 0.67 | XPC | 4.14 | 1.09E−03 | 0.73 | REG1A | 2.95 | 5.89E−02 | 2.97 | MB21D2 | 2.35 | 2.49E−01 | 0.23 |
| MS4A4E | 9.21 | 6.52E−18 | 0.89 | CD86 | 4.13 | 1.14E−03 | 0.37 | SP110 | 2.94 | 5.89E−02 | 0.37 | PNISR | 2.35 | 2.46E−01 | 0.14 |
| LPAR6 | 9.17 | 9.88F−18 | 1.01 | TET2 | 4.13 | 1.14E−03 | 0.28 | DACH1 | 2.93 | 6.10E−02 | 0.94 | MPPED2 | 2.34 | 2.54E−01 | 1.13 |
| ST6GALNAC3 | 9.17 | 9.98E−18 | 0.98 | TAPT1 | 4.12 | 1.18E−03 | 0.58 | ELMO1-AS1 | 2.93 | 6.11E−02 | 0.52 | FAM78A | 2.34 | 2.54E−01 | 0.66 |
| FOXN3 | 9.11 | 1.68E−17 | 0.25 | ARHGAP26 | 4.12 | 1.18E−03 | 0.05 | IRS2 | 2.93 | 6.15E−02 | 0.33 | PQLC1 | 2.34 | 2.56E−01 | 0.27 |
| FCGR2A | 8.92 | 9.24E−17 | 0.64 | SIGLEC1 | 4.11 | 1.25E−03 | 0.59 | SPECC1 | 2.93 | 6.13E−02 | 0.28 | SNX8 | 2.34 | 2.56E−01 | 0.23 |
| XAF1 | 8.88 | 1.31E−16 | 0.94 | SETX | 4.11 | 1.24E−03 | 0.27 | FEZ2 | 2.93 | 6.09E−02 | 0.25 | FILIP1L | 2.34 | 2.52E−01 | 0.2 |
| ARHGAP22 | 8.83 | 2.04E−16 | 0.79 | ARRB2 | 4.08 | 1.40E−03 | 0.34 | RPTOR | 2.93 | 6.14E−02 | 0.23 | VAV1 | 2.34 | 2.52E−01 | 0.2 |
| RAB31 | 8.8 | 2.52E−16 | 0.37 | CHST15 | 4.07 | 1.47E−03 | 0.39 | BLNK | 2.92 | 6.26E−02 | 0.47 | PIGF | 2.33 | 2.58E−01 | 0.32 |
| SIPA1L1 | 8.74 | 4.17E−16 | 0.31 | EPC2 | 4.07 | 1.44E−03 | 0.37 | CLIP2 | 2.92 | 6.22E−02 | 0.43 | UNC93B1 | 2.33 | 2.61E−01 | 0.3 |
| BCL2 | 8.65 | 9.28E−16 | 0.65 | AGBL4 | 4.06 | 1.51E−03 | 1.38 | TIGAR | 2.92 | 6.29E−02 | 0.32 | MS4A4A | 2.33 | 2.57E−01 | 0.21 |
| HCLS1 | 8.59 | 1.48E−15 | 0.64 | CPQ | 4.06 | 1.48E−03 | 0.27 | ABCC4 | 2.92 | 6.33E−02 | 0.25 | PRKCH | 2.33 | 2.61E−01 | 0.16 |
| CEL | 8.57 | 1.72E−15 | 5.71 | MERTK | 4.05 | 1.59E−03 | 0.25 | AGPAT3 | 2.92 | 6.22E−02 | 0.24 | VWCE | 2.32 | 2.64E−01 | 3.51 |
| RNF130 | 8.53 | 2.50E−15 | 0.38 | NR5A2 | 4.03 | 1.70E−03 | 1.52 | ALOX15B | 2.91 | 6.52E−02 | 0.39 | MEIS2 | 2.32 | 2.64E−01 | 0.46 |
| PREX1 | 8.51 | 2.91E−15 | 0.6 | MAF | 4.03 | 1.69E−03 | 0.79 | TRPM3 | 2.9 | 6.62E−02 | 1.49 | PPMID | 2.32 | 2.63E−01 | 0.28 |
| IGSF21 | 8.47 | 4.20E−15 | 1.52 | CREBL2 | 4.03 | 1.70E−03 | 0.51 | REV1 | 2.9 | 6.68E−02 | 0.24 | BCL2L13 | 2.32 | 2.63E−01 | 0.27 |
| MSR1 | 8.38 | 8.48E−15 | 0.5 | SPOP | 4.01 | 1.81E−03 | 0.36 | SLC22A23 | 2.89 | 6.86E−02 | 0.53 | FGF14 | 2.31 | 2.69E−01 | 1.46 |
| F13A1 | 8.28 | 1.97E−14 | 1 | SFSWAP | 4 | 1.89E−03 | 0.38 | EMSY | 2.89 | 6.84E−02 | 0.3 | TNFRSF11A | 2.31 | 2.72E−01 | 0.63 |
| CSF3R | 8.27 | 2.08E−14 | 0.76 | GAB2 | 4 | 1.90E−03 | 0.16 | HGSNAT | 2.89 | 6.79E−02 | 0.23 | GLIPR1 | 2.31 | 2.70E−01 | 0.4 |
| CPB1 | 8.19 | 4.03E−14 | 5.66 | EIF2AK4 | 3.99 | 1.97E−03 | 0.39 | SENP5 | 2.89 | 6.84E−02 | 0.22 | DPP7 | 2.31 | 2.68E−01 | 0.36 |
| AKAP13 | 8.05 | 1.23E−13 | 0.15 | EYA2 | 3.98 | 2.05E−03 | 0.63 | RAB30-AS1 | 2.88 | 6.94E−02 | 0.55 | VPS35 | 2.31 | 2.70E−01 | 0.2 |
| ITSN1 | 8 | 1.73E−13 | 0.54 | ARHGEF6 | 3.98 | 2.08E−03 | 0.45 | INPP5A | 2.88 | 7.04E−02 | 0.49 | USP24 | 2.31 | 2.70E−01 | 0.16 |
| ZFHX3 | 7.96 | 2.29E−13 | 0.49 | PHACTR1 | 3.98 | 2.03E−03 | 0.31 | AC124312.1 | 2.88 | 6.96E−02 | 0.44 | WDFY3 | 2.31 | 2.70E−01 | 0.15 |
| LCP2 | 7.87 | 4.72E−13 | 0.51 | P2RY8 | 3.95 | 2.28E−03 | 0.66 | DDX10 | 2.87 | 7.12E−02 | 0.28 | ABHD6 | 2.3 | 2.77E−01 | 0.37 |
| SRGAP2C | 7.81 | 7.27E−13 | 0.8 | RNF135 | 3.95 | 2.29E−03 | 0.56 | SRRM2 | 2.87 | 7.18E−02 | 0.21 | ZC3H18 | 2.3 | 2.74E−01 | 0.31 |
| IQGAP2 | 7.81 | 7.75E−13 | 0.4 | PLXDC2 | 3.91 | 2.71E−03 | −0.12 | DNMT3A | 2.86 | 7.38E−02 | 0.27 | GALNT11 | 2.3 | 2.77E−01 | 0.21 |
| INPP5D | 7.74 | 1.32E−12 | 0.51 | SDK1 | 3.9 | 2.77E−03 | 0.56 | CSF2RA | 2.86 | 7.51E−02 | 0.17 | TBCD | 2.3 | 2.78E−01 | 0.18 |
| TBC1D14 | 7.6 | 3.65E−12 | 0.52 | TGFB1 | 3.9 | 2.84E−03 | 0.43 | SAP30 | 2.85 | 7.56E−02 | 0.47 | PCTP | 2.29 | 2.83E−01 | 0.57 |
| SH3TC1 | 7.38 | 1.78E−11 | 0.64 | PABPC4 | 3.89 | 2.93E−03 | 0.43 | ETV5 | 2.85 | 7.51E−02 | 0.41 | PHKA2 | 2.29 | 2.81E−01 | 0.35 |
| CSF1R | 7.35 | 2.17E−11 | 0.64 | SCAPER | 3.89 | 2.87E−03 | 0.3 | ALG13 | 2.85 | 7.51E−02 | 0.25 | APH1B | 2.29 | 2.83E−01 | 0.25 |
| AP2A2 | 7.35 | 2.29E−11 | 0.5 | GRAMD1B | 3.88 | 3.06E−03 | 0.29 | ZNF33A | 2.85 | 7.60E−02 | 0.23 | NDUFA10 | 2.29 | 2.81E−01 | 0.23 |
| CIITA | 7.32 | 2.72E−11 | 0.52 | RIMS2 | 3.87 | 3.10E−03 | 1.88 | VENTX | 2.84 | 7.78E−02 | 1.1 | GALM | 2.29 | 2.83E−01 | 0.21 |
| GCG | 7.28 | 3.65E−11 | 3.57 | IKZF1 | 3.87 | 3.08E−03 | 0.29 | SF3B1 | 2.84 | 7.76E−02 | 0.22 | YIPF4 | 2.29 | 2.80E−01 | 0.17 |
| KCNQ1 | 7.28 | 3.73E−11 | 0.81 | PARP14 | 3.87 | 3.09E−03 | 0.27 | RIN3 | 2.84 | 7.92E−02 | 0.18 | RPS6KC1 | 2.29 | 2.83E−01 | 0.17 |
| HLA-DPB1 | 7.21 | 5.90E−11 | 0.66 | PPFIBP2 | 3.86 | 3.24E−03 | 0.33 | SCTR | 2.83 | 8.13E−02 | 1.54 | TMEM71 | 2.28 | 2.87E−01 | 0.7 |
| CPA1 | 7.11 | 1.20E−10 | 6.57 | FMNL1 | 3.86 | 3.24E−03 | 0.33 | RGS18 | 2.83 | 8.02E−02 | 0.75 | DNASE1 | 2.28 | 2.90E−01 | 0.46 |
| RAPH1 | 7.09 | 1.37E−10 | 0.65 | PAPOLG | 3.85 | 3.32E−03 | 0.4 | CCDC30 | 2.83 | 7.93E−02 | 0.74 | ZNF562 | 2.28 | 2.90E−01 | 0.22 |
| MEGF9 | 7.06 | 1.72E−10 | 0.73 | LILRB3 | 3.84 | 3.44E−03 | 0.57 | NIN | 2.82 | 8.22E−02 | 0.22 | DCDC2 | 2.27 | 2.96E−01 | 1.4 |
| GABPB1-AS1 | 7.02 | 2.16E−10 | 1.17 | PFKFB3 | 3.84 | 3.53E−03 | 0.29 | LY96 | 2.81 | 8.55E−02 | 0.37 | FPR1 | 2.27 | 2.91E−01 | 0.29 |
| PALD1 | 7.01 | 2.32E−10 | 0.68 | TIMM23B | 3.83 | 3.58E−03 | 0.42 | RGS10 | 2.81 | 8.53E−02 | 0.28 | KLHL8 | 2.27 | 2.94E−01 | 0.24 |
| TNFRSF1B | 6.99 | 2.65E−10 | 0.62 | UBXN2B | 3.83 | 3.64E−03 | 0.34 | REPS2 | 2.8 | 8.59E−02 | 0.31 | ATP6V1C1 | 2.27 | 2.90E−01 | 0.17 |
| FGD4 | 6.96 | 3.21E−10 | 0.25 | GNG2 | 3.82 | 3.71E−03 | 0.6 | MILR1 | 2.79 | 8.92E−02 | 0.64 | SNTG1 | 2.26 | 2.98E−01 | 1.56 |
| MUC6 | 6.87 | 6.15E−10 | 2.71 | PELI1 | 3.82 | 3.70E−03 | 0.29 | UBA6-AS1 | 2.79 | 8.95E−02 | 0.46 | HTR7 | 2.26 | 2.98E−01 | 0.85 |
| ZNF846 | 6.84 | 7.33E−10 | 0.78 | SYNE3 | 3.81 | 3.86E−03 | 0.86 | TMEM106A | 2.79 | 8.92E−02 | 0.44 | ZNF385D | 2.26 | 2.98E−01 | 0.54 |
| DTNA | 6.83 | 7.92E−10 | 0.55 | NOTCH2 | 3.81 | 3.83E−03 | 0.32 | CAMK2G | 2.79 | 8.85E−02 | 0.35 | ZFP14 | 2.26 | 3.01E−01 | 0.52 |
| RCSD1 | 6.79 | 1.04E−09 | 0.56 | ADCY7 | 3.81 | 3.90E−03 | 0.3 | CRLF3 | 2.79 | 8.98E−02 | 0.22 | GPR155 | 2.26 | 2.98E−01 | 0.25 |
| MALAT1 | 6.79 | 1.07E−09 | 0.1 | PDE7B | 3.8 | 3.98E−03 | 0.87 | FAM49A | 2.79 | 8.92E−02 | 0.19 | RPRD2 | 2.26 | 3.00E−01 | 0.19 |
| MRC1 | 6.77 | 1.18E−09 | 0.46 | FMNL3 | 3.8 | 3.96E−03 | 0.36 | SIRT5 | 2.77 | 9.37E−02 | 0.47 | CNST | 2.26 | 2.97E−01 | 0.18 |
| ERICH1 | 6.76 | 1.28E−09 | 0.71 | ZMAT1 | 3.79 | 4.16E−03 | 0.59 | ITFG2 | 2.77 | 9.32E−02 | 0.38 | KLHL5 | 2.25 | 3.05E−01 | 0.19 |
| SH2B3 | 6.76 | 1.23E−09 | 0.56 | IFI44 | 3.79 | 4.11E−03 | 0.46 | KRCC1 | 2.77 | 9.44E−02 | 0.37 | SEC63 | 2.25 | 3.06E−01 | 0.18 |
| FPR3 | 6.72 | 1.65E−09 | 0.7 | SOX6 | 3.77 | 4.40E−03 | 0.75 | ERCC6L2 | 2.77 | 9.47E−02 | 0.27 | ZNF106 | 2.25 | 3.06E−01 | 0.17 |
| CMTM7 | 6.71 | 1.71E−09 | 0.6 | APAF1 | 3.77 | 4.50E−03 | 0.41 | ADGRE2 | 2.77 | 9.42E−02 | 0.25 | VPS13C | 2.25 | 3.05E−01 | 0.12 |
| SH3KBP1 | 6.71 | 1.73E−09 | 0.48 | CXCR4 | 3.76 | 4.65E−03 | 0.51 | MTMR10 | 2.77 | 9.42E−02 | 0.23 | NR4A3 | 2.24 | 3.15E−01 | 0.86 |
| WBP1L | 6.65 | 2.44E−09 | 0.63 | TBL1X | 3.73 | 5.11E−03 | 0.42 | FCGR2B | 2.76 | 9.58E−02 | 0.46 | PADI2 | 2.24 | 3.15E−01 | 0.45 |
| AFF3 | 6.54 | 4.99E−09 | 0.74 | ANKS1A | 3.73 | 5.06E−03 | 0.23 | RELL1 | 2.76 | 9.58E−02 | 0.3 | CCDC88B | 2.24 | 3.15E−01 | 0.41 |
| MEF2A | 6.53 | 5.20E−09 | 0.19 | LILRB2 | 3.72 | 5.44E−03 | 0.9 | WDFY4 | 2.76 | 9.55E−02 | 0.2 | MEF2D | 2.24 | 3.15E−01 | 0.29 |
| LRRK2 | 6.44 | 9.87E−09 | 0.49 | ANKRD44 | 3.72 | 5.43E−03 | 0.14 | CADPS | 2.75 | 9.90E−02 | 1.1 | HAVCR2 | 2.24 | 3.10E−01 | 0.18 |
| CCND3 | 6.42 | 1.11E−08 | 0.48 | HSPA6 | 3.71 | 5.45E−03 | 0.84 | ZC3H12D | 2.75 | 9.80E−02 | 0.49 | SNX29 | 2.24 | 3.10E−01 | 0.07 |
| SH3BP5 | 6.37 | 1.52E−08 | 0.55 | FES | 3.71 | 5.61E−03 | 0.51 | RGS12 | 2.75 | 9.78E−02 | 0.23 | ARRDC5 | 2.23 | 3.17E−01 | 0.52 |
| SSH2 | 6.36 | 1.61E−08 | 0.15 | LAT2 | 3.71 | 5.51E−03 | 0.44 | CREBBP | 2.75 | 9.94E−02 | 0.2 | ARL14EP | 2.23 | 3.19E−01 | 0.48 |
| ATP8B4 | 6.35 | 1.70E−08 | 0.42 | DPP6 | 3.7 | 5.65E−03 | 1.16 | PRLR | 2.74 | 1.01E−01 | 1.09 | SZT2 | 2.23 | 3.21E−01 | 0.31 |
| SUMF1 | 6.35 | 1.66E−08 | 0.37 | NEGR1 | 3.7 | 5.73E−03 | 0.82 | SCLT1 | 2.74 | 1.01E−01 | 0.25 | SLC25A26 | 2.23 | 3.20E−01 | 0.22 |
| HERPUD1 | 6.33 | 1.85E−08 | 0.53 | TCAIM | 3.7 | 5.66E−03 | 0.48 | CNTN5 | 2.73 | 1.02E−01 | 1.47 | MTMR12 | 2.22 | 3.25E−01 | 0.32 |
| PSMA1 | 6.29 | 2.46E−08 | 0.87 | LUC7L | 3.67 | 6.30E−03 | 0.34 | SERTAD2 | 2.73 | 1.03E−01 | 0.34 | GAS6 | 2.22 | 3.25E−01 | 0.31 |
| MX2 | 6.28 | 2.50E−08 | 1.07 | LRRC37A3 | 3.67 | 6.41E−03 | 0.32 | FGGY | 2.73 | 1.04E−01 | 0.25 | RNF4 | 2.22 | 3.26E−01 | 0.25 |
| ADAP2 | 6.27 | 2.78E−08 | 0.5 | AKNA | 3.66 | 6.56E−03 | 0.36 | ZNF516 | 2.73 | 1.04E−01 | 0.22 | RIN2 | 2.22 | 3.22E−01 | 0.21 |
| RGS2 | 6.17 | 4.98E−08 | 1.09 | TRPS1 | 3.66 | 6.55E−03 | 0.08 | ZNF577 | 2.72 | 1.05E−01 | 0.39 | TTYH3 | 2.22 | 3.25E−01 | 0.18 |
| DPEP2 | 6.17 | 5.01E−08 | 0.81 | DNAJC5 | 3.65 | 6.78E−03 | 0.3 | TRIM56 | 2.72 | 1.04E−01 | 0.31 | LPGAT1 | 2.22 | 3.25E−01 | 0.18 |
| PILRA | 6.17 | 4.92E−08 | 0.67 | SLC30A8 | 3.62 | 7.66E−03 | 2.74 | SPATA6 | 2.72 | 1.07E−01 | 0.29 | PRKD3 | 2.22 | 3.26E−01 | 0.17 |
| DOCK2 | 6.16 | 5.13E−08 | 0.25 | SPEN | 3.62 | 7.53E−03 | 0.31 | BICC1 | 2.71 | 1.07E−01 | 0.43 | PJA2 | 2.22 | 3.22E−01 | 0.15 |
| L3MBTL4 | 6.15 | 5.55E−08 | 0.53 | MDM4 | 3.6 | 8.20E−03 | 0.31 | GAS2L3 | 2.71 | 1.08E−01 | 0.27 | DSE | 2.21 | 3.29E−01 | 0.27 |
| CTRB1 | 6.14 | 5.69E−08 | 5.76 | FLRT2 | 3.59 | 8.45E−03 | 1.05 | CHST9 | 2.7 | 1.11E−01 | 1.55 | SLC7A2 | 2.2 | 3.36E−01 | 1.91 |
| IFI44L | 6.1 | 7.56E−08 | 0.96 | RYR1 | 3.57 | 9.05E−03 | 1.54 | IQSEC2 | 2.7 | 1.12E−01 | 0.32 | PAK3 | 2.2 | 3.37E−01 | 1.56 |
| PARVB | 6.06 | 9.28E−08 | 0.52 | SETBP1 | 3.57 | 8.85E−03 | 0.87 | MAPK8IP3 | 2.7 | 1.11E−01 | 0.3 | MAPK10 | 2.2 | 3.39E−01 | 0.51 |
| CREB5 | 5.99 | 1.40E−07 | 0.66 | KIAA0319L | 3.57 | 8.97E−03 | 0.31 | CA5B | 2.7 | 1.10E−01 | 0.29 | ZNF333 | 2.2 | 3.39E−01 | 0.5 |
| PHF20 | 5.99 | 1.40E−07 | 0.45 | CLEC4E | 3.56 | 9.35E−03 | 1.64 | PPCDC | 2.69 | 1.13E−01 | 0.47 | ZMAT3 | 2.2 | 3.35E−01 | 0.44 |
| NHSL1 | 5.98 | 1.48E−07 | 0.37 | FAM228B | 3.56 | 9.35E−03 | 0.61 | ZNF75D | 2.69 | 1.15E−01 | 0.37 | CRYL1 | 2.2 | 3.37E−01 | 0.25 |
| CASS4 | 5.95 | 1.74E−07 | 1.1 | PDE4A | 3.55 | 9.67E−03 | 0.46 | KIAA1328 | 2.69 | 1.14E−01 | 0.29 | SSBP3 | 2.2 | 3.36E−01 | 0.23 |
| PNLIPRP1 | 5.89 | 2.46E−07 | 6.82 | GPRIN3 | 3.55 | 9.70E−03 | 0.24 | LINC00662 | 2.68 | 1.17E−01 | 0.48 | UBE2G2 | 2.2 | 3.39E−01 | 0.19 |
| DAGLB | 5.89 | 2.46E−07 | 0.59 | PNLIP | 3.54 | 9.90E−03 | 6.22 | PLEKHO1 | 2.68 | 1.18E−01 | 0.43 | YAF2 | 2.2 | 3.34E−01 | 0.17 |
| C1orf162 | 5.87 | 2.71E−07 | 0.9 | PRDM1 | 3.54 | 9.76E−03 | 0.65 | SLC2A9 | 2.68 | 1.16E−01 | 0.23 | MPRIP | 2.2 | 3.39E−01 | 0.17 |
| IL10RA | 5.84 | 3.32E−07 | 0.62 | USP39 | 3.54 | 9.78E−03 | 0.5 | LONRF3 | 2.67 | 1.19E−01 | 0.63 | SIPA1L3 | 2.2 | 3.39E−01 | 0.15 |
| FLI1 | 5.84 | 3.22E−07 | 0.38 | SNRNP70 | 3.53 | 1.01E−02 | 0.33 | TRANK1 | 2.67 | 1.20E−01 | 0.4 | C3AR1 | 2.19 | 3.47E−01 | 0.34 |
| TPCN1 | 5.82 | 3.64E−07 | 0.54 | RBMS3 | 3.52 | 1.07E−02 | 0.87 | PRKAR2B | 2.66 | 1.24E−01 | 0.49 | ZNF33B | 2.19 | 3.48E−01 | 0.31 |
| POU2F2 | 5.8 | 4.01E−07 | 0.88 | THEMIS2 | 3.52 | 1.05E−02 | 0.31 | NFATC2 | 2.66 | 1.22E−01 | 0.25 | MX1 | 2.18 | 3.48E−01 | 0.41 |
| FRMD4B | 5.8 | 4.03E−07 | 0.29 | PRSS1 | 3.5 | 1.13E−02 | 5.2 | PER1 | 2.66 | 1.24E−01 | 0.22 | ENOX2 | 2.18 | 3.55E−01 | 0.21 |
| PLXNA2 | 5.79 | 4.22E−07 | 0.78 | SCIMP | 3.5 | 1.14E−02 | 0.57 | SETD3 | 2.66 | 1.22E−01 | 0.2 | PLEKHM1 | 2.18 | 3.55E−01 | 0.2 |
| FOXP1 | 5.79 | 4.37E−07 | 0.09 | RUFY3 | 3.5 | 1.14E−02 | 0.26 | UBAC2 | 2.66 | 1.23E−01 | 0.19 | PRUNE2 | 2.17 | 3.58E−01 | 0.73 |
| KIAA0513 | 5.74 | 5.71E−07 | 0.84 | ESRRG | 3.49 | 1.18E−02 | 2 | PIGL | 2.65 | 1.27E−01 | 0.32 | HACD4 | 2.17 | 3.57E−01 | 0.56 |
| TRIM22 | 5.67 | 8.48E−07 | 0.58 | RAP1GAP2 | 3.49 | 1.15E−02 | 0.47 | ATXN7L1 | 2.65 | 1.27E−01 | 0.19 | SLC15A2 | 2.17 | 3.57E−01 | 0.37 |
| MAP2K1 | 5.67 | 8.48E−07 | 0.41 | LATS2 | 3.49 | 1.17E−02 | 0.43 | C6 | 2.64 | 1.29E−01 | 2.63 | EIF2B3 | 2.17 | 3.57E−01 | 0.23 |
| MYO1F | 5.67 | 8.27E−07 | 0.34 | SLCO2B1 | 3.49 | 1.18E−02 | 0.17 | KCNB2 | 2.63 | 1.32E−01 | 2.22 | MCM9 | 2.17 | 3.61E−01 | 0.23 |
| NBEA | 5.65 | 9.38E−07 | 0.67 | CCDC149 | 3.48 | 1.21E−02 | 0.86 | FAM102B | 2.63 | 1.33E−01 | 0.2 | CYP20A1 | 2.17 | 3.55E−01 | 0.18 |
| SAMHD1 | 5.61 | 1.19E−06 | 0.34 | DIP2C | 3.47 | 1.25E−02 | 0.49 | NFYC | 2.62 | 1.37E−01 | 0.25 | FAM185A | 2.16 | 3.62E−01 | 0.27 |
| RBM6 | 5.61 | 1.14E−06 | 0.31 | JDP2 | 3.47 | 1.24E−02 | 0.42 | ASH1L | 2.62 | 1.37E−01 | 0.12 | ZNF780B | 2.16 | 3.62E−01 | 0.25 |
| SEC14L1 | 5.56 | 1.49E−06 | 0.42 | C3 | 3.46 | 1.26E−02 | 0.73 | ASCC3 | 2.61 | 1.40E−01 | 0.19 | HNRNPL | 2.16 | 3.66E−01 | 0.23 |
| SPTLC2 | 5.54 | 1.73E−06 | 0.42 | NFKBID | 3.46 | 1.29E−02 | 0.72 | ERCC3 | 2.6 | 1.42E−01 | 0.47 | GGA1 | 2.16 | 3.69E−01 | 0.17 |
| MIR646HG | 5.5 | 2.08E−06 | 0.69 | RUBCN | 3.46 | 1.26E−02 | 0.37 | ATP10A | 2.6 | 1.44E−01 | 0.44 | CACNA1A | 2.15 | 3.71E−01 | 1.14 |
| TGFBI | 5.49 | 2.25E−06 | 0.51 | DMD | 3.44 | 1.37E−02 | 0.58 | B3GNTL1 | 2.6 | 1.42E−01 | 0.43 | IQCH-AS1 | 2.15 | 3.74E−01 | 0.53 |
| CREM | 5.47 | 2.44E−06 | 0.55 | SLC18B1 | 3.44 | 1.35E−02 | 0.53 | GFOD1 | 2.6 | 1.45E−01 | 0.31 | RBM19 | 2.15 | 3.74E−01 | 0.34 |
| FGR | 5.47 | 2.40E−06 | 0.53 | ARRDC2 | 3.44 | 1.36E−02 | 0.49 | ZNF445 | 2.59 | 1.48E−01 | 0.55 | MRPL3 | 2.15 | 3.70E−01 | 0.32 |
| TAPT1-AS1 | 5.45 | 2.68E−06 | 1.12 | SMAP2 | 3.44 | 1.37E−02 | 0.2 | CRK | 2.59 | 1.48E−01 | 0.26 | RNF144B | 2.15 | 3.71E−01 | 0.15 |
| MVB12B | 5.4 | 3.59E−06 | 0.5 | FAM111A | 3.43 | 1.41E−02 | 0.32 | LRRC37B | 2.59 | 1.47E−01 | 0.24 | KDM6A | 2.15 | 3.72E−01 | 0.15 |
| MPHOSPH6 | 5.37 | 4.05E−06 | 0.66 | JAK2 | 3.43 | 1.38E−02 | 0.25 | DENNDIA | 2.59 | 1.48E−01 | 0.13 | LINC00106 | 2.14 | 3.81E−01 | 0.43 |
| MAP3K14 | 5.36 | 4.41E−06 | 0.55 | NAALADL2 | 3.42 | 1.46E−02 | 0.3 | PDE3A | 2.58 | 1.49E−01 | 0.5 | ZNF490 | 2.14 | 3.79E−01 | 0.26 |
| CLEC2D | 5.36 | 4.28E−06 | 0.54 | ATM | 3.42 | 1.43E−02 | 0.23 | SLC37A2 | 2.58 | 1.51E−01 | 0.49 | KAT2B | 2.14 | 3.83E−01 | 0.19 |
| ZFP36L2 | 5.35 | 4.53E−06 | 0.4 | ATG7 | 3.42 | 1.42E−02 | 0.03 | C5AR1 | 2.58 | 1.50E−01 | 0.29 | HNRNPUL1 | 2.14 | 3.80E−01 | 0.18 |
| SRGN | 5.35 | 4.57E−06 | 0.38 | SENP6 | 3.41 | 1.51E−02 | 0.24 | VASH1 | 2.58 | 1.48E−01 | 0.25 | ANP32A | 2.14 | 3.81E−01 | 0.17 |
| TNFSF8 | 5.32 | 5.27E−06 | 1.26 | TLR5 | 3.4 | 1.55E−02 | 0.58 | DENND4B | 2.57 | 1.53E−01 | 0.27 | NOL4 | 2.13 | 3.87E−01 | 2.92 |
| PRKX | 5.32 | 5.35E−06 | 0.65 | KIAA1551 | 3.4 | 1.55E−02 | 0.29 | BBS9 | 2.57 | 1.55E−01 | 0.24 | FAM149A | 2.13 | 3.86E−01 | 0.42 |
| JAML | 5.32 | 5.27E−06 | 0.58 | KIF16B | 3.4 | 1.56E−02 | 0.26 | SHMT1 | 2.56 | 1.56E−01 | 0.6 | ZNF189 | 2.13 | 3.88E−01 | 0.42 |
| CHPT1 | 5.3 | 5.99E−06 | 0.48 | EAF2 | 3.39 | 1.60E−02 | 0.6 | APIS2 | 2.56 | 1.56E−01 | 0.28 | CWF19L2 | 2.13 | 3.86E−01 | 0.22 |
| MDM2 | 5.27 | 6.88E−06 | 0.52 | PPM1L | 3.38 | 1.66E−02 | 0.32 | SPG7 | 2.56 | 1.56E−01 | 0.23 | STXBP5 | 2.13 | 3.88E−01 | 0.17 |
| STK17B | 5.26 | 7.23E−06 | 0.48 | SON | 3.38 | 1.63E−02 | 0.24 | LAPTM5 | 2.56 | 1.57E−01 | 0.19 | CDKN1A | 2.12 | 3.98E−01 | 0.62 |
| MS4A14 | 5.24 | 7.81E−06 | 1.05 | PUDP | 3.37 | 1.70E−02 | 0.89 | ERCC8 | 2.55 | 1.61E−01 | 0.34 | FAM110B | 2.12 | 3.98E−01 | 0.32 |
| CACNB2 | 5.23 | 8.55E−06 | 1.29 | TCP11L1 | 3.37 | 1.70E−02 | 0.56 | KIAA0232 | 2.55 | 1.61E−01 | 0.22 | CATSPER2 | 2.12 | 3.96E−01 | 0.31 |
| NLRP3 | 5.2 | 9.78E−06 | 1.07 | SLC43A2 | 3.37 | 1.68E−02 | 0.31 | AAK1 | 2.55 | 1.63E−01 | 0.15 | USP16 | 2.12 | 3.93E−01 | 0.25 |
| RGL1 | 5.2 | 9.81E−06 | 0.49 | LRRTM4 | 3.36 | 1.74E−02 | 1.94 | NAGK | 2.54 | 1.64E−01 | 0.33 | TANGO6 | 2.12 | 3.93E−01 | 0.17 |
| ST6GAL1 | 5.2 | 9.68E−06 | 0.39 | NPIPB5 | 3.36 | 1.74E−02 | 0.29 | IVD | 2.53 | 1.68E−01 | 0.53 | C6orf62 | 2.12 | 3.92E−01 | 0.16 |
| IFI16 | 5.2 | 9.93E−06 | 0.39 | LMF1 | 3.35 | 1.82E−02 | 0.51 | ADGRG6 | 2.53 | 1.68E−01 | 0.32 | ARHGEF2 | 2.12 | 3.97E−01 | 0.16 |
| OGFRL1 | 5.19 | 1.04E−05 | 0.45 | XRRA1 | 3.34 | 1.86E−02 | 0.45 | PPP1R10 | 2.53 | 1.70E−01 | 0.28 | STIM2 | 2.12 | 3.94E−01 | 0.12 |
| PARVG | 5.17 | 1.13E−05 | 0.42 | RFX3 | 3.34 | 1.85E−02 | 0.28 | NAMPT | 2.53 | 1.68E−01 | 0.23 | JADE1 | 2.11 | 4.03E−01 | 0.42 |
| REV3L | 5.17 | 1.15E−05 | 0.41 | MTHFR | 3.33 | 1.92E−02 | 0.55 | PMS1 | 2.53 | 1.67E−01 | 0.21 | CTNNA2 | 2.1 | 4.12E−01 | 1.49 |
| CUZD1 | 5.12 | 1.41E−05 | 4.34 | DNMBP | 3.33 | 1.90E−02 | 0.5 | SF1 | 2.53 | 1.69E−01 | 0.21 | RNASE6 | 2.1 | 4.10E−01 | 1.48 |
| PDE4B | 5.12 | 1.45E−05 | 0.34 | NFIA | 3.32 | 1.96E−02 | 0.27 | LONP2 | 2.53 | 1.69E−01 | 0.18 | IRF7 | 2.1 | 4.07E−01 | 0.71 |
| TRPM2 | 5.11 | 1.51E−05 | 0.4 | SYAP1 | 3.31 | 2.02E−02 | 0.55 | KCNAB1 | 2.52 | 1.73E−01 | 0.73 | PRPSAP1 | 2.1 | 4.08E−01 | 0.35 |
| DDB2 | 5.09 | 1.66E−05 | 0.66 | AKAP7 | 3.3 | 2.12E−02 | 0.53 | STPG2 | 2.52 | 1.72E−01 | 0.52 | PCGF3 | 2.1 | 4.14E−01 | 0.2 |
| SLC2A13 | 5.07 | 1.85E−05 | 0.44 | ME2 | 3.3 | 2.12E−02 | 0.28 | CROCC | 2.52 | 1.72E−01 | 0.5 | AZIN1-AS1 | 2.09 | 4.23E−01 | 0.73 |
| FAM120B | 5.06 | 1.97E−05 | 0.41 | ATRN | 3.3 | 2.12E−02 | 0.24 | MSANTD2 | 2.52 | 1.71E−01 | 0.44 | CRADD | 2.09 | 4.17E−01 | 0.26 |
| TLR1 | 5.02 | 2.36E−05 | 0.75 | ZC3H3 | 3.29 | 2.14E−02 | 0.4 | REG3A | 2.51 | 1.75E−01 | 3.09 | RBAK-RBAKDN | 2.09 | 4.21E−01 | 0.25 |
| DLEU2 | 4.99 | 2.67E−05 | 0.16 | NCOR2 | 3.29 | 2.15E−02 | 0.24 | MOB1B | 2.51 | 1.76E−01 | 0.2 | BRD1 | 2.09 | 4.18E−01 | 0.23 |
| CD163L1 | 4.96 | 3.18E−05 | 0.52 | CPM | 3.29 | 2.14E−02 | 0.18 | KDM3B | 2.51 | 1.76E−01 | 0.19 | SGMS1 | 2.09 | 4.19E−01 | 0.17 |
| SNX6 | 4.96 | 3.14E−05 | 0.42 | HEXDC | 3.28 | 2.22E−02 | 0.58 | CNTN4 | 2.5 | 1.80E−01 | 1 | GAB3 | 2.09 | 4.19E−01 | 0.14 |
| MAP3K8 | 4.96 | 3.13E−05 | 0.41 | KCTD12 | 3.28 | 2.22E−02 | 0.47 | SIRPB2 | 2.5 | 1.79E−01 | 0.23 | TSIX | 2.08 | 4.28E−01 | 1.97 |
| TLR2 | 4.96 | 3.18E−05 | 0.3 | KCNE1 | 3.28 | 2.20E−02 | 0.34 | SYNE1 | 2.5 | 1.79E−01 | 0.21 | HGF | 2.08 | 4.24E−01 | 0.66 |
| DEPTOR | 4.95 | 3.26E−05 | 0.77 | CTTNBP2 | 3.27 | 2.30E−02 | 0.87 | ATP9B | 2.5 | 1.78E−01 | 0.16 | TRMT1 | 2.08 | 4.28E−01 | 0.56 |
| WNT2B | 4.92 | 3.84E−05 | 0.83 | DAP | 3.27 | 2.31E−02 | 0.42 | FAM19A2 | 2.49 | 1.82E−01 | 0.59 | FRMD3 | 2.08 | 4.25E−01 | 0.41 |
| ITGA4 | 4.92 | 3.76E−05 | 0.54 | SERINC5 | 3.27 | 2.30E−02 | 0.25 | MAGT1 | 2.49 | 1.82E−01 | 0.33 | CXorf21 | 2.08 | 4.26E−01 | 0.39 |
| TNRC18 | 4.92 | 3.76E−05 | 0.35 | HS6ST3 | 3.26 | 2.35E−02 | 2.32 | TTBK2 | 2.49 | 1.83E−01 | 0.22 | RPL31 | 2.08 | 4.26E−01 | 0.36 |
| PSTPIP2 | 4.91 | 4.05E−05 | 0.46 | NUDT16 | 3.26 | 2.33E−02 | 0.82 | SENP7 | 2.49 | 1.85E−01 | 0.2 | SMYD4 | 2.08 | 4.29E−01 | 0.31 |
| INSR | 4.88 | 4.45E−05 | 0.39 | PIGX | 3.26 | 2.32E−02 | 0.74 | ZNF75A | 2.48 | 1.87E−01 | 0.55 | ARID1A | 2.08 | 4.28E−01 | 0.15 |
| NLRP1 | 4.85 | 5.12E−05 | 0.49 | PTPN18 | 3.26 | 2.35E−02 | 0.32 | ZRSR2 | 2.48 | 1.88E−01 | 0.42 | IL18R1 | 2.07 | 4.33E−01 | 0.62 |
| TACC1 | 4.85 | 5.16E−05 | 0.37 | MEF2C-AS1 | 3.25 | 2.43E−02 | 0.76 | ZBTB40 | 2.48 | 1.89E−01 | 0.4 | GPR75-ASB3 | 2.07 | 4.34E−01 | 0.35 |
| PELI2 | 4.84 | 5.49E−05 | 0.58 | ZDHHC14 | 3.25 | 2.45E−02 | 0.29 | ESF1 | 2.48 | 1.89E−01 | 0.34 | MARS | 2.07 | 4.33E−01 | 0.25 |
| RILPL2 | 4.82 | 5.96E−05 | 0.62 | IL13RA1 | 3.25 | 2.45E−02 | 0.29 | TMC8 | 2.48 | 1.86E−01 | 0.32 | STK40 | 2.07 | 4.32E−01 | 0.22 |
| CACNA2D3 | 4.81 | 6.27E−05 | 1.47 | TTC17 | 3.25 | 2.43E−02 | 0.22 | TMCO4 | 2.48 | 1.86E−01 | 0.28 | PDPK1 | 2.07 | 4.36E−01 | 0.16 |
| LINC00937 | 4.79 | 6.78E−05 | 0.7 | ERI3 | 3.24 | 2.47E−02 | 0.41 | TESK2 | 2.48 | 1.88E−01 | 0.23 | ERGIC1 | 2.07 | 4.30E−01 | 0.15 |
| DLG2 | 4.77 | 7.46E−05 | 0.72 | PRKACB | 3.24 | 2.51E−02 | 0.35 | THADA | 2.48 | 1.87E−01 | 0.19 | RABEP1 | 2.07 | 4.33E−01 | 0.15 |
| ANKH | 4.77 | 7.45E−05 | 0.43 | REL | 3.24 | 2.50E−02 | 0.22 | CIRBP | 2.47 | 1.92E−01 | 0.25 | COX19 | 2.06 | 4.45E−01 | 0.38 |
| ERO1B | 4.76 | 7.90E−05 | 0.68 | ARHGEF40 | 3.22 | 2.62E−02 | 0.46 | ENTPD1-AS1 | 2.47 | 1.90E−01 | 0.22 | TECPR2 | 2.06 | 4.44E−01 | 0.36 |
| CEBPD | 4.76 | 7.82E−05 | 0.51 | TRAPPC12 | 3.22 | 2.64E−02 | 0.27 | MAN1A1 | 2.47 | 1.90E−01 | 0.16 | ARHGAP4 | 2.06 | 4.44E−01 | 0.36 |
| SLC22A15 | 4.75 | 8.22E−05 | 0.46 | PLSCR1 | 3.22 | 2.62E−02 | 0.24 | FRAS1 | 2.46 | 1.94E−01 | 0.84 | METTL25 | 2.06 | 4.39E−01 | 0.3 |
| MOB3A | 4.74 | 8.49E−05 | 0.59 | MBP | 3.22 | 2.64E−02 | 0.24 | LILRB5 | 2.46 | 1.94E−01 | 0.83 | NSL1 | 2.06 | 4.42E−01 | 0.22 |
| TRAF3IP2-AS1 | 4.74 | 8.47E−05 | 0.52 | SH3BGRL | 3.22 | 2.62E−02 | 0.23 | RCAN1 | 2.46 | 1.96E−01 | 0.37 | NFAM1 | 2.06 | 4.42E−01 | 0.22 |
| TTR | 4.73 | 8.97E−05 | 3.5 | ARHGAP15 | 3.22 | 2.66E−02 | 0.04 | ZNF251 | 2.46 | 1.96E−01 | 0.34 | SRGAP2-AS1 | 2.05 | 4.51E−01 | 0.71 |
| RCBTB2 | 4.73 | 8.90E−05 | 0.51 | DHX34 | 3.21 | 2.74E−02 | 0.49 | ZNF791 | 2.46 | 1.94E−01 | 0.31 | GKAP1 | 2.05 | 4.47E−01 | 0.56 |
| IFNGR2 | 4.72 | 9.17E−05 | 0.35 | PLTP | 3.2 | 2.78E−02 | 0.89 | TMLHE | 2.46 | 1.97E−01 | 0.21 | GYPC | 2.05 | 4.47E−01 | 0.55 |
| CR1 | 4.71 | 9.83E−05 | 0.9 | MPC1 | 3.2 | 2.82E−02 | 0.58 | SLC1A3 | 2.46 | 1.96E−01 | 0.07 | ASCC1 | 2.05 | 4.47E−01 | 0.18 |
| PEAK1 | 4.71 | 9.62E−05 | 0.33 | CALCOCO2 | 3.2 | 2.78E−02 | 0.42 | PLB1 | 2.45 | 2.01E−01 | 0.61 | KPNA6 | 2.05 | 4.48E−01 | 0.18 |
| HLA-DPA1 | 4.69 | 1.03E−04 | 0.72 | LINC01504 | 3.18 | 3.02E−02 | 0.69 | C12orf45 | 2.45 | 2.01E−01 | 0.58 | AC159540.1 | 2.04 | 4.53E−01 | 3.98 |
| BDP1 | 4.65 | 1.26E−04 | 0.41 | CPED1 | 3.18 | 3.02E−02 | 0.38 | ARL17B | 2.45 | 1.98E−01 | 0.31 | TMEM9B-AS1 | 2.04 | 4.57E−01 | 0.82 |
| SSBP2 | 4.65 | 1.25E−04 | 0.39 | SP100 | 3.18 | 2.98E−02 | 0.21 | TM9SF4 | 2.45 | 2.00E−01 | 0.27 | LINC00886 | 2.04 | 4.59E−01 | 0.59 |
| CTNND2 | 4.64 | 1.35E−04 | 1.91 | C20orf194 | 3.18 | 2.98E−02 | 0.15 | CCDC170 | 2.44 | 2.04E−01 | 0.37 | CD93 | 2.04 | 4.54E−01 | 0.38 |
| CARD8 | 4.64 | 1.34E−04 | 0.4 | PDGFD | 3.17 | 3.06E−02 | 1.33 | KIAA0100 | 2.44 | 2.05E−01 | 0.28 | PRKAG1 | 2.04 | 4.56E−01 | 0.33 |
| ZNF710 | 4.64 | 1.35E−04 | 0.35 | AC012368.1 | 3.17 | 3.09E−02 | 0.98 | IL6R | 2.44 | 2.05E−01 | 0.25 | MAD1L1 | 2.04 | 4.53E−01 | 0.16 |
| GARNL3 | 4.61 | 1.49E−04 | 0.98 | ZNF263 | 3.17 | 3.11E−02 | 0.87 | DUSP16 | 2.44 | 2.05E−01 | 0.24 | USP4 | 2.04 | 4.55E−01 | 0.13 |
| LY86 | 4.61 | 1.54E−04 | 0.53 | CERS4 | 3.16 | 3.19E−02 | 0.94 | CELA3A | 2.43 | 2.09E−01 | 3.69 | TTLL7 | 2.03 | 4.65E−01 | 1.72 |
| GNG7 | 4.6 | 1.56E−04 | 0.56 | ESR1 | 3.16 | 3.14E−02 | 0.38 | AK9 | 2.43 | 2.10E−01 | 0.79 | ZNF41 | 2.03 | 4.67E−01 | 0.66 |
| FGL2 | 4.54 | 2.07E−04 | 1.06 | ABAT | 3.15 | 3.27E−02 | 0.51 | ASTN2 | 2.43 | 2.11E−01 | 0.79 | IL16 | 2.03 | 4.70E−01 | 0.61 |
| MAP3K3 | 4.54 | 2.10E−04 | 0.39 | MR1 | 3.13 | 3.49E−02 | 0.54 | SIN3B | 2.43 | 2.09E−01 | 0.44 | AHDC1 | 2.02 | 4.79E−01 | 0.53 |
| SNCA | 4.49 | 2.62E−04 | 0.64 | PRKCD | 3.13 | 3.48E−02 | 0.36 | APPL2 | 2.43 | 2.11E−01 | 0.2 | SPATA7 | 2.02 | 4.72E−01 | 0.38 |
| RHBDF2 | 4.49 | 2.57E−04 | 0.36 | FRMD4A | 3.13 | 3.49E−02 | 0.07 | LYVE1 | 2.42 | 2.14E−01 | 1.92 | ZC3H7A | 2.02 | 4.71E−01 | 0.16 |
| | | | | | | | | | | | | PLCL2 | 2.02 | 4.79E−01 | 0.14 |
| | | | | | | | | | | | | AMY2A | 2.01 | 4.87E−01 | 7.45 |
| | | | | | | | | | | | | DNHD1 | 2.01 | 4.81E−01 | 0.33 |
| | | | | | | | | | | | | USP10 | 2.01 | 4.82E−01 | 0.14 |
| | | | | | | | | | | | | TM7SF3 | 2 | 4.91E−01 | 0.18 |
| | | | | | | | | | | | | HEXB | 2 | 4.95E−01 | 0.15 |

Abbreviations: FC = fold change

TABLE 2.6
Malignant by treatment

| | | | Log2fold- | | | | Log2fold- | | | | Log2fold- | | | | Log2fold- |
|---|---|---|---|---|---|---|---|---|---|---|---|---|---|---|---|
| Gene | Score | Pval | change | Gene | Score | Pval | change | Gene | Score | Pval | change | Gene | Score | Pval | change |
| ZNF83 | 40.95 | 0.00E+00 | 1.84 | CLDN16 | 6.23 | 4.47E−09 | 1.15 | ITGB1BP1 | 4.11 | 1.99E−04 | 0.18 | MRPS18B | 2.86 | 1.50E−02 | 0.08 |
| TJP2 | 37.53 | 1.84E−304 | 3.06 | ARHGAP26-AS1 | 6.23 | 4.24E−09 | 0.8 | CANT1 | 4.11 | 2.05E−04 | 0.15 | LONRF3 | 2.86 | 1.51E−02 | 0.08 |
| USP39 | 37.29 | 8.96E−301 | 1.98 | KAT8 | 6.23 | 4.25E−09 | 0.5 | KRCC1 | 4.11 | 2.03E−04 | 0.13 | PSD4 | 2.86 | 1.48E−02 | 0.06 |
| HDAC9 | 36.65 | 1.45E−290 | 1.96 | FCGBP | 6.22 | 4.67E−09 | 2.19 | DYNC2LI1 | 4.11 | 2.02E−04 | 0.12 | PLEKHS1 | 2.86 | 1.51E−02 | −0.01 |
| TGM2 | 34.15 | 2.66E−252 | 1.75 | COMP | 6.22 | 4.50E−09 | 1.96 | CACFD1 | 4.1 | 2.15E−04 | 0.8 | ZBED3-AS1 | 2.85 | 1.56E−02 | 4.4 |
| HLA-B | 34 | 4.59E−250 | 1.88 | GPR173 | 6.22 | 4.51E−09 | 0.98 | LRRTM4 | 4.1 | 2.11E−04 | 0.78 | MANIC1 | 2.85 | 1.54E−02 | 1.02 |
| GABRP | 30.22 | 1.62E−197 | 1.77 | AGR3 | 6.22 | 4.74E−09 | 0.41 | C1orf21 | 4.1 | 2.14E−04 | 0.26 | DXO | 2.85 | 1.54E−02 | 1.02 |
| TMSB10 | 29.98 | 2.35E−194 | 1.4 | KIAA1841 | 6.22 | 4.53E−09 | 0.34 | ASAH1 | 4.1 | 2.14E−04 | 0.24 | LURAP1 | 2.85 | 1.57E−02 | 0.86 |
| POU5F1 | 29.04 | 2.03E−182 | 1.71 | TTR | 6.21 | 4.87E−09 | 2.76 | TANGO6 | 4.1 | 2.14E−04 | 0.16 | ADTRP | 2.85 | 1.57E−02 | 0.52 |
| XIST | 28.10 | 8.01E−172 | 3.24 | TRPM3 | 6.21 | 4.91E−09 | 1.06 | EIF4A1 | 4.1 | 2.14E−04 | 0.11 | ZNF519 | 2.85 | 1.53E−02 | 0.36 |
| ACTN4 | 27.67 | 1.23E−165 | 0.93 | ARCN1 | 6.21 | 4.96E−09 | 0.41 | MORN4 | 4.09 | 2.24E−04 | 0.66 | F8 | 2.85 | 1.55E−02 | 0.36 |
| ZNF804A | 27.6 | 9.06E−165 | 5.86 | SRGAP1 | 6.21 | 4.89E−09 | 0.26 | ZNF43 | 4.09 | 2.23E−04 | 0.52 | HDAC11 | 2.85 | 1.54E−02 | 0.26 |
| C19orf33 | 26.76 | 7.47E−155 | 2.55 | SHC3 | 6.2 | 5.12E−09 | 0.98 | PYROXD2 | 4.09 | 2.20E−04 | 0.42 | IFT46 | 2.85 | 1.56E−02 | 0.19 |
| MT2A | 26.72 | 1.88E−154 | 2.23 | FAM96B | 6.2 | 5.28E−09 | 0.82 | IAH1 | 4.09 | 2.20E−04 | 0.35 | POMT1 | 2.85 | 1.54E−02 | 0.15 |
| ZFAND3 | 26.65 | 1.31E−153 | 0.65 | CLPTM1 | 6.2 | 5.18E−09 | 0.49 | RRAGC | 4.09 | 2.23E−04 | 0.34 | NCK1 | 2.85 | 1.54E−02 | 0.11 |
| RPL28 | 26.34 | 3.64E−150 | 1.66 | BTG1 | 6.2 | 5.23E−09 | 0.37 | NSMF | 4.09 | 2.21E−04 | 0.21 | NARS2 | 2.85 | 1.53E−02 | 0.07 |
| LINC01322 | 25.34 | 6.62E−139 | 1.77 | CRLF1 | 6.19 | 5.61E−09 | 1.27 | WSB2 | 4.09 | 2.20E−04 | 0.16 | PPCS | 2.85 | 1.55E−02 | 0.06 |
| LRP4 | 25.22 | 1.18E−137 | 1.77 | UBXN6 | 6.19 | 5.37E−09 | 0.66 | DENR | 4.09 | 2.23E−04 | 0.14 | TRIO | 2.85 | 1.56E−02 | 0.06 |
| ACTB | 25.14 | 1.01E−136 | 1.08 | DESI1 | 6.19 | 5.57E−09 | 0.57 | CERS5 | 4.09 | 2.24E−04 | 0.13 | ZNF710 | 2.85 | 1.54E−02 | 0.04 |
| FKBP10 | 25.04 | 1.07E−135 | 2.72 | XXYLT1 | 6.19 | 5.63E−09 | 0.45 | PDE5A | 4.09 | 2.16E−04 | 0.09 | STK35 | 2.85 | 1.55E−02 | 0.03 |
| FTH1 | 24.84 | 1.57E−133 | 1.33 | HINT1 | 6.19 | 5.67E−09 | 0.41 | SAR1A | 4.09 | 2.18E−04 | 0.08 | UPF1 | 2.85 | 1.53E−02 | 0 |
| AC159540.1 | 24.49 | 8.74E−130 | 4.33 | PSMD7 | 6.19 | 5.57E−09 | 0.33 | SAA1 | 4.08 | 2.32E−04 | 0.8 | KDM6B | 2.85 | 1.56E−02 | 0 |
| SYT1 | 24.44 | 2.48E−129 | 3.6 | LTBP3 | 6.19 | 5.68E−09 | 0.29 | FBP1 | 4.08 | 2.25E−04 | 0.26 | KIF27 | 2.85 | 1.53E−02 | −0.01 |
| NCALD | 24.14 | 4.03E−126 | 1.66 | UBE2L6 | 6.18 | 5.84E−09 | 1.28 | TM2D3 | 4.08 | 2.25E−04 | 0.25 | ARAP3 | 2.85 | 1.56E−02 | −0.02 |
| DDIT4 | 23.53 | 6.81E−120 | 1.01 | SIAH2 | 6.18 | 5.75E−09 | 0.49 | PIP5K1C | 4.08 | 2.31E−04 | 0.19 | RELL1 | 2.85 | 1.56E−02 | −0.09 |
| RPL13A | 23.47 | 2.62E−119 | 1.35 | ATP6V0D1 | 6.18 | 6.02E−09 | 0.45 | AMMECR1 | 4.08 | 2.25E−04 | 0.17 | GDPGP1 | 2.84 | 1.61E−02 | 2.46 |
| CCND1 | 23.24 | 6.79E−117 | 1.51 | RRP12 | 6.18 | 5.86E−09 | 0.32 | PRDX6 | 4.08 | 2.28E−04 | 0.17 | MRAS | 2.84 | 1.58E−02 | 1.53 |
| KRT19 | 23.08 | 2.76E−115 | 0.56 | CAMK4 | 6.17 | 6.15E−09 | 1.29 | GSN | 4.08 | 2.30E−04 | 0.15 | PSAT1 | 2.84 | 1.57E−02 | 1.48 |
| CCND3 | 23.05 | 4.78E−115 | 0.97 | LYPLA2 | 6.17 | 6.07E−09 | 0.67 | TSEN34 | 4.07 | 2.42E−04 | 0.82 | CFAP73 | 2.84 | 1.61E−02 | 0.49 |
| PMEPA1 | 23 | 1.53E−114 | 0.85 | PCSK7 | 6.17 | 6.20E−09 | 0.28 | ALG1 | 4.07 | 2.39E−04 | 0.7 | MAPKAPK3 | 2.84 | 1.58E−02 | 0.32 |
| RPLP1 | 22.75 | 4.50E−112 | 1.5 | CLTA | 6.17 | 6.39E−09 | 0.28 | HOXB-AS3 | 4.07 | 2.40E−04 | 0.57 | ICA1L | 2.84 | 1.60E−02 | 0.3 |
| RPL35 | 22.65 | 4.48E−111 | 1.5 | COPE | 6.16 | 6.79E−09 | 0.66 | APH1A | 4.07 | 2.38E−04 | 0.33 | PGAP3 | 2.84 | 1.60E−02 | 0.18 |
| RPLP2 | 22.63 | 6.80E−111 | 1.37 | PSMD8 | 6.16 | 6.56E−09 | 0.52 | D2HGDH | 4.07 | 2.35E−04 | 0.19 | LINC01106 | 2.84 | 1.59E−02 | 0.17 |
| ZC3H11A | 22.45 | 4.02E−109 | 2.6 | UBE3B | 6.16 | 6.56E−09 | 0.45 | SUSD1 | 4.07 | 2.41E−04 | 0.18 | ETAA1 | 2.84 | 1.57E−02 | 0.11 |
| IFI44L | 22.45 | 3.80E−109 | 1.9 | ST3GAL2 | 6.16 | 6.65E−09 | 0.44 | WBP4 | 4.07 | 2.36E−04 | 0.16 | UFC1 | 2.84 | 1.60E−02 | 0.11 |
| CD59 | 22.25 | 3.27E−107 | 1.69 | SLC16A4 | 6.16 | 6.63E−09 | 0.42 | RBAK-RBAKDN | 4.07 | 2.37E−04 | 0.16 | EXTL2 | 2.84 | 1.59E−02 | 0.05 |
| RPS19 | 22.09 | 1.07E−105 | 1.34 | GNB1L | 6.15 | 7.13E−09 | 0.94 | YES1 | 4.07 | 2.35E−04 | 0.14 | MIER2 | 2.84 | 1.61E−02 | 0.04 |
| NAV2 | 21.69 | 1.00E−104 | 1.19 | FUNDC2 | 6.15 | 7.25E−09 | 0.78 | MFF | 4.07 | 2.40E−04 | 0.14 | TECPR2 | 2.84 | 1.59E−02 | 0.02 |
| ALDOA | 21.98 | 1.07E−104 | 3.27 | AGFG2 | 6.15 | 6.93E−09 | 0.56 | RNF217-AS1 | 4.06 | 2.47E−04 | 1.59 | FOXP4 | 2.84 | 1.60E−02 | 0.01 |
| KRT8 | 21.77 | 1.09E−102 | 0.98 | EIF2S2 | 6.15 | 6.96E−09 | 0.37 | IDH3B | 4.06 | 2.45E−04 | 0.6 | TMEM242 | 2.34 | 1.58E−02 | 0 |
| IFI27 | 21.7 | 4.59E−102 | 1.69 | PABPC4 | 6.15 | 6.88E−09 | 0.32 | SMC1A | 4.06 | 2.51E−04 | 0.18 | UGDH | 2.84 | 1.59E−02 | 0 |
| ZBTB20 | 21.7 | 5.32E−102 | 0.43 | MRPS21 | 6.15 | 6.86E−09 | 0.3 | CCDC191 | 4.06 | 2.49E−04 | 0.16 | IFNAR1 | 2.84 | 1.59E−02 | −0.07 |
| RPL13 | 21.53 | 1.89E−100 | 0.99 | SCO2 | 6.14 | 7.33E−09 | 3.54 | PSMA5 | 4.06 | 2.47E−04 | 0.14 | MRPL21 | 2.83 | 1.66E−02 | 0.6 |
| CSNK1E | 21.04 | 6.58E−96 | 1.51 | PSMB8 | 6.14 | 7.66E−09 | 0.93 | FAM118B | 4.06 | 2.50E−04 | 0.13 | TBL3 | 2.83 | 1.62E−02 | 0.57 |
| IFI6 | 20.96 | 3.10E−95 | 2.59 | AACS | 6.14 | 7.32E−09 | 0.5 | MAP2K2 | 4.06 | 2.48E−04 | 0.12 | SNX10 | 2.83 | 1.65E−02 | 0.29 |
| PKHD1 | 20.66 | 1.74E−92 | 1.34 | RPS3 | 6.14 | 7.45E−09 | 0.32 | ACADS | 4.05 | 2.55E−04 | 0.82 | CCDC59 | 2.83 | 1.65E−02 | 0.24 |
| LDLRAD4 | 20.61 | 4.94E−92 | 1.04 | CCDC144NL-AS1 | 6.13 | 8.03E−09 | 1.16 | PLA2G15 | 4.05 | 2.59E−04 | 0.79 | AIFM2 | 2.83 | 1.63E−02 | 0.22 |
| MAML2 | 20.59 | 6.30E−92 | 0.67 | HMGCL | 6.13 | 7.81E−09 | 0.71 | TMEM129 | 4.05 | 2.58E−04 | 0.7 | CCDC58 | 2.83 | 1.65E−02 | 0.09 |
| ILIRAPL2 | 20.55 | 1.44E−91 | 1.75 | PSMC5 | 6.12 | 8.19E−09 | 0.95 | TMEM9 | 4.05 | 2.57E−04 | 0.58 | PRMT7 | 2.83 | 1.66E−02 | 0.09 |
| TMSB4X | 20.51 | 3.68E−91 | 0.92 | RELB | 6.12 | 8.46E−09 | 0.35 | VMA21 | 4.05 | 2.58E−04 | 0.5 | PQLC3 | 2.83 | 1.62E−02 | 0.08 |
| SLCO3A1 | 20.46 | 9.43E−91 | 0.99 | TANC2 | 6.12 | 8.19E−09 | 0.22 | TTLL12 | 4.05 | 2.57E−04 | 0.29 | KLHL12 | 2.83 | 1.63E−02 | 0.05 |
| TRIM29 | 20.35 | 9.67E−90 | 1.3 | VAT1 | 6.11 | 8.77E−09 | 1.26 | IFT27 | 4.05 | 2.60E−04 | 0.26 | IDH1 | 2.83 | 1.63E−02 | 0.01 |
| PLCB1 | 20.33 | 1.26E−89 | 1.15 | GEM | 6.11 | 8.71E−09 | 0.94 | PITHD1 | 4.05 | 2.62E−04 | 0.25 | TPGS2 | 2.83 | 1.62E−02 | 0 |
| PARP9 | 20.23 | 9.56E−89 | 1.22 | CLPTM1L | 6.11 | 8.84E−09 | 0.38 | USP20 | 4.05 | 2.54E−04 | 0.23 | RMND5B | 2.83 | 1.63E−02 | 0 |
| SOD2 | 20.14 | 5.52E−88 | 2.82 | SLC9A3R2 | 6.1 | 9.60E−09 | 0.54 | S100A11 | 4.05 | 2.63E−04 | 0.19 | HECA | 2.83 | 1.66E−02 | −0.05 |
| RPS16 | 20.05 | 3.95E−87 | 1.32 | MYLK | 6.1 | 9.79E−09 | 0.51 | ZFP64 | 4.05 | 2.59E−04 | 0.13 | DNPH1 | 2.82 | 1.70E−02 | 0.75 |
| IQCH | 19.9 | 6.93E−86 | 1.5 | RAB8B | 6.1 | 9.25E−09 | 0.28 | HKR1 | 4.05 | 2.55E−04 | 0.08 | UNG | 2.82 | 1.69E−02 | 0.54 |
| EDF1 | 19.82 | 3.22E−85 | 1.23 | COCH | 6.09 | 1.03E−08 | 0.93 | KCTD12 | 4.04 | 2.66E−04 | 0.93 | CUEDC2 | 2.82 | 1.71E−02 | 0.5 |
| LY6E | 19.74 | 1.79E−84 | 2.15 | TMC8 | 6.08 | 1.10E−08 | 1.12 | GRWD1 | 4.04 | 2.70E−04 | 0.89 | ZNF324 | 2.82 | 1.67E−02 | 0.48 |
| RPL41 | 19.62 | 1.61E−83 | 1.23 | GADD45GIP1 | 6.08 | 1.07E−08 | 0.94 | VPS18 | 4.04 | 2.73E−04 | 0.85 | DAXX | 2.82 | 1.70E−02 | 0.4 |
| RRBP1 | 19.33 | 4.49E−81 | 0.93 | SNRPB2 | 6.08 | 1.05E−08 | 0.8 | PBXIP1 | 4.04 | 2.66E−04 | 0.28 | ITGAM | 2.82 | 1.70E−02 | 0.37 |
| CTSH | 19.27 | 1.55E−80 | 1.78 | DDA1 | 6.08 | 1.10E−08 | 0.52 | PTOV1 | 4.04 | 2.69E−04 | 0.27 | WDR31 | 2.82 | 1.71E−02 | 0.34 |
| IGF2BP3 | 19.17 | 1.07E−79 | 0.89 | NPIPB4 | 6.08 | 1.08E−08 | 0.49 | GTF3A | 4.04 | 2.70E−04 | 0.26 | PQBP1 | 2.82 | 1.67E−02 | 0.31 |
| RPL36 | 19 | 2.57E−78 | 1.24 | SLC35F1 | 6.07 | 1.14E−08 | 0.99 | TRAF3IP2 | 4.04 | 2.70E−04 | 0.26 | MSANTD4 | 2.82 | 1.68E−02 | 0.19 |
| IFNAR2 | 18.85 | 4.33E−77 | 3.82 | SLC7A6OS | 6.07 | 1.13E−08 | 0.52 | THOC3 | 4.04 | 2.70E−04 | 0.22 | LDLRAP1 | 2.82 | 1.69E−02 | 0.14 |
| PKM | 18.84 | 4.65E−77 | 0.8 | SKP1 | 6.07 | 1.15E−08 | 0.33 | TAF1D | 4.04 | 2.70E−04 | 0.18 | MRPL43 | 2.82 | 1.67E−02 | 0.12 |
| RAD50 | 18.82 | 7.31E−77 | 2.97 | CHFR | 6.07 | 1.14E−08 | 0.22 | RNF185 | 4.04 | 2.74E−04 | 0.13 | GALNT10 | 2.82 | 1.67E−02 | 0.12 |
| KCTD7 | 18.69 | 8.14E−76 | 2.88 | HOXB2 | 6.06 | 1.25E−08 | 2.32 | EPAS1 | 4.04 | 2.70E−04 | 0.09 | TBX15 | 2.82 | 1.70E−02 | 0.11 |
| BMPR1B | 18.65 | 1.58E−75 | 1.25 | ANKDD1B | 6.06 | 1.23E−08 | 0.7 | COX19 | 4.04 | 2.66E−04 | 0.08 | SLC4A1AP | 2.82 | 1.70E−02 | 0.05 |
| HLA-C | 18.6 | 4.34E−75 | 1.28 | RAB9A | 6.06 | 1.23E−08 | 0.62 | IL12RB2 | 4.03 | 2.80E−04 | 1.27 | AP3M1 | 2.82 | 1.70E−02 | −0.06 |
| BCL2L1 | 18.51 | 2.33E−74 | 0.71 | DPP10 | 6.06 | 1.22E−08 | 0.57 | LIPE | 4.03 | 2.80E−04 | 1.03 | NXT1 | 2.81 | 1.72E−02 | 1 |
| CALD1 | 18.4 | 1.74E−73 | 1.13 | CBX3 | 6.06 | 1.20E−08 | 0.3 | CYCS | 4.03 | 2.80E−04 | 0.4 | FILIP1 | 2.81 | 1.74E−02 | 0.97 |
| RPS17 | 18.27 | 1.83E−72 | 2.43 | PAQR8 | 6.05 | 1.28E−08 | 2.9 | RPL22L1 | 4.03 | 2.85E−04 | 0.34 | NR1H4 | 2.81 | 1.72E−02 | 0.76 |
| SERF2 | 18.19 | 8.03E−72 | 1.14 | SLC39A4 | 6.05 | 1.32E−08 | 1.38 | DNAJA2 | 4.03 | 2.76E−04 | 0.33 | MALRD1 | 2.81 | 1.76E−02 | 0.71 |
| C9orf16 | 18.18 | 9.92E−72 | 2.07 | C12orf65 | 6.05 | 1.29E−08 | 0.82 | TTLL4 | 4.03 | 2.77E−04 | 0.18 | COMMD5 | 2.81 | 1.73E−02 | 0.4 |
| AC058791.1 | 18.18 | 9.60E−72 | 1.61 | THBS2 | 6.05 | 1.27E−08 | 0.7 | VOPP1 | 4.03 | 2.82E−04 | 0.17 | HS3ST5 | 2.81 | 1.73E−02 | 0.39 |
| NCOR2 | 18.15 | 1.75E−71 | 0.8 | KCNMB4 | 6.05 | 1.26E−08 | 0.68 | SECISBP2L | 4.03 | 2.77E−04 | 0.16 | ARL4A | 2.81 | 1.73E−02 | 0.3 |
| PARL | 18.12 | 2.68E−71 | 3.21 | DUSP3 | 6.05 | 1.25E−08 | 0.5 | PFDN2 | 4.03 | 2.78E−04 | 0.15 | FAM174A | 2.81 | 1.72E−02 | 0.29 |
| BIRC3 | 18.09 | 4.70E−71 | 1.17 | GTF2A2 | 6.05 | 1.27E−08 | 0.4 | EIF3L | 4.03 | 2.82E−04 | 0.13 | C14orf119 | 2.81 | 1.74E−02 | 0.28 |
| AC019117.2 | 18.03 | 1.36E−70 | 3.38 | NUDT4 | 6.05 | 1.28E−08 | 0.33 | EYA2 | 4.03 | 2.85E−04 | 0.03 | USP30 | 2.81 | 1.75E−02 | 0.28 |
| CA1 | 17.99 | 3.02E−70 | 1.86 | CRY1 | 6.05 | 1.30E−08 | 0.19 | MEA1 | 4.02 | 2.89E−04 | 1.41 | IFITM2 | 2.81 | 1.75E−02 | 0.23 |
| PLAT | 17.93 | 8.48E−70 | 1.61 | CLEC19A | 6.04 | 1.35E−08 | 1.46 | TSKU | 4.02 | 2.97E−04 | 1.4 | BTN3A1 | 2.81 | 1.75E−02 | 0.2 |
| MYOF | 17.72 | 3.35E−68 | 0.58 | U2AF1 | 6.04 | 1.36E−08 | 1.27 | MED19 | 4.02 | 2.91E−04 | 0.63 | CHRNA7 | 2.81 | 1.72E−02 | 0.18 |
| MICAL2 | 17.71 | 4.07E−68 | 0.86 | MRPS26 | 6.04 | 1.35E−08 | 0.88 | MIR762HG | 4.02 | 2.95E−04 | 0.49 | CPOX | 2.81 | 1.73E−02 | 0.1 |
| GTF2IRD1 | 17.63 | 1.55E−67 | 0.83 | ARSJ | 6.04 | 1.35E−08 | 0.46 | FAM213B | 4.02 | 2.97E−04 | 0.39 | ILIRAPL1 | 2.81 | 1.71E−02 | 0.07 |
| RPS27A | 17.59 | 3.43E−67 | 1.14 | SCGB2B2 | 6.04 | 1.33E−08 | 0.34 | FAM3A | 4.02 | 2.89E−04 | 0.34 | SLC22A3 | 2.81 | 1.75E−02 | 0.07 |
| SMCR5 | 17.53 | 9.00E−67 | 1.71 | DGUOK | 6.04 | 1.36E−08 | 0.34 | NIPA1 | 4.02 | 2.92E−04 | 0.18 | ACP1 | 2.81 | 1.72E−02 | 0.03 |
| MYH11 | 17.5 | 1.70E−66 | 1.74 | MAGT1 | 6.03 | 1.44E−08 | 0.27 | H2AFY | 4.02 | 2.94E−04 | 0.16 | LSM12 | 2.81 | 1.73E−02 | 0.02 |
| PIK3C2G | 17.46 | 2.91E−66 | 0.77 | STK40 | 6.03 | 1.47E−08 | 0.26 | AC007319.1 | 4.02 | 2.92E−04 | 0.11 | CRYAB | 2.8 | 1.79E−02 | 3.54 |
| SNHG19 | 17.37 | 1.54E−65 | 1.59 | GLG1 | 6.03 | 1.45E−08 | 0.23 | ATP8A2 | 4.01 | 3.00E−04 | 0.95 | NOS1 | 2.8 | 1.78E−02 | 1.34 |
| MAPK10 | 17.35 | 2.11E−65 | 1.06 | COL26A1 | 6.02 | 1.56E−08 | 1.84 | TPRG1L | 4.01 | 2.97E−04 | 0.7 | LINC01023 | 2.8 | 1.80E−02 | 0.86 |
| RPL38 | 17.3 | 5.04E−65 | 1.69 | RILPL2 | 6.02 | 1.52E−08 | 0.83 | ENKUR | 4.01 | 3.01E−04 | 0.3 | MRPL10 | 2.8 | 1.79E−02 | 0.69 |
| MBOAT7 | 17.28 | 7.23E−65 | 1.38 | ZBTB4 | 6.02 | 1.55E−08 | 0.32 | CHMP4C | 4.01 | 3.00E−04 | 0.26 | AATK | 2.8 | 1.77E−02 | 0.43 |
| RPL27A | 17.23 | 1.49E−64 | 1.08 | PKD2 | 6.02 | 1.55E−08 | 0.31 | COMMD1 | 4.01 | 2.98E−04 | 0.18 | FAM110C | 2.8 | 1.80E−02 | 0.26 |
| LCN2 | 17.13 | 9.22E−64 | 1.03 | RNF121 | 6.02 | 1.56E−08 | 0.31 | LIPH | 4.01 | 2.99E−04 | 0.16 | PSMD1 | 2.8 | 1.79E−02 | 0.12 |
| SPDYE16 | 17.05 | 3.55E−63 | 2.07 | TMEM67 | 6.02 | 1.56E−08 | 0.3 | RNF149 | 4.01 | 3.03E−04 | 0.16 | SLC12A9 | 2.8 | 1.79E−02 | 0.07 |
| MX2 | 17.03 | 5.19E−63 | 1.78 | ACTN1 | 6.02 | 1.58E−08 | 0.28 | DGCR6L | 4 | 3.21E−04 | 1.32 | ELAC2 | 2.8 | 1.80E−02 | 0.06 |
| MLXIP | 16.9 | 4.56E−62 | 0.88 | WDR46 | 6.01 | 1.60E−08 | 0.68 | SDF2L1 | 4 | 3.15E−04 | 0.98 | BTBD1 | 2.8 | 1.80E−02 | 0.04 |
| ZNF544 | 16.84 | 1.24E−61 | 3.43 | ROR2 | 6.01 | 1.61E−08 | 0.67 | GMPR | 4 | 3.11E−04 | 0.7 | TFE3 | 2.8 | 1.76E−02 | 0.02 |
| FKBP5 | 16.8 | 2.11E−61 | 0.69 | EXOC3L4 | 6.01 | 1.65E−08 | 0.55 | ROGDI | 4 | 3.19E−04 | 0.62 | AL450992.2 | 2.79 | 1.82E−02 | 2.12 |
| ZNF665 | 16.79 | 2.91E−61 | 1.72 | MPZL2 | 6.01 | 1.63E−08 | 0.42 | UBA7 | 4 | 3.13E−04 | 0.61 | GABBR2 | 2.79 | 1.86E−02 | 1.62 |
| PPDPF | 16.76 | 4.67E−61 | 1.37 | LACTB2 | 6.01 | 1.60E−08 | 0.41 | NIT2 | 4 | 3.21E−04 | 0.54 | TMEM169 | 2.79 | 1.83E−02 | 1.05 |
| ANXA1 | 16.73 | 7.30E−61 | 0.71 | SYAP1 | 6.01 | 1.58E−08 | 0.37 | LINC01285 | 4 | 3.09E−04 | 0.44 | LINC00908 | 2.79 | 1.83E−02 | 1.02 |
| ARPC1A | 16.71 | 9.88E−61 | 3.04 | MAP3K9 | 6.01 | 1.61E−08 | 0.3 | ARHGAP23 | 4 | 3.13E−04 | 0.22 | POP4 | 2.79 | 1.83E−02 | 0.85 |
| KRT18 | 16.61 | 5.36E−60 | 0.84 | LDHB | 6 | 1.75E−08 | 1.25 | UBE4B | 4 | 3.12E−04 | 0.17 | KATNB1 | 2.79 | 1.84E−02 | 0.56 |
| CAMKID | 16.6 | 5.90E−60 | 1.02 | ATXN7L3B | 6 | 1.74E−08 | 0.8 | RRP1B | 4 | 3.11E−04 | 0.16 | HMGA2 | 2.79 | 1.81E−02 | 0.28 |
| MAP3K13 | 16.57 | 9.62E−60 | 0.61 | REXO2 | 6 | 1.76E−08 | 0.49 | KHDRBS1 | 4 | 3.18E−04 | 0.16 | CDC34 | 2.79 | 1.84E−02 | 0.27 |
| CD82 | 16.53 | 2.14E−59 | 1.29 | GOLT1B | 6 | 1.70E−08 | 0.42 | GFM1 | 4 | 3.18E−04 | 0.11 | PDK4 | 2.79 | 1.83E−02 | 0.17 |
| PSAP | 16.51 | 2.84E−59 | 1.15 | KIF7 | 5.99 | 1.81E−08 | 1.04 | MRPL54 | 3.99 | 3.23E−04 | 1.18 | GSK3B | 2.79 | 1.81E−02 | 0.13 |
| CRIP2 | 16.5 | 3.21E−59 | 1.13 | SORCS2 | 5.99 | 1.87E−08 | 1.02 | BAK1 | 3.99 | 3.22E−04 | 0.8 | NAGLU | 2.79 | 1.82E−02 | 0.1 |
| ZFHX3 | 16.43 | 9.60E−59 | 0.68 | LINC00578 | 5.99 | 1.83E−08 | 0.95 | PPM1N | 3.99 | 3.34E−04 | 0.68 | LHFPL3 | 2.79 | 1.84E−02 | 0.05 |
| MT-CO1 | 16.41 | 1.41E−58 | 0.83 | MT-CO2 | 5.99 | 1.83E−08 | 0.37 | NUBP1 | 3.99 | 3.27E−04 | 0.52 | ANP32B | 2.79 | 1.82E−02 | 0.04 |
| AXL | 16.38 | 2.26E−58 | 1.6 | MIEN1 | 5.98 | 1.91E−08 | 1.11 | GPS1 | 3.99 | 3.30E−04 | 0.39 | RNF215 | 2.79 | 1.82E−02 | 0.03 |
| RANBP17 | 16.32 | 6.32E−58 | 0.74 | RRP36 | 5.98 | 1.95E−08 | 0.49 | GLI2 | 3.99 | 3.22E−04 | 0.23 | ZFP90 | 2.79 | 1.81E−02 | 0.02 |
| THSD4 | 16.26 | 1.71E−57 | 0.57 | RALYL | 5.98 | 1.97E−08 | 0.49 | ADCY6 | 3.99 | 3.23E−04 | 0.16 | MLF2 | 2.79 | 1.82E−02 | −0.01 |
| MX1 | 16.25 | 1.80E−57 | 1.49 | MROH6 | 5.98 | 1.89E−08 | 0.35 | IFT122 | 3.99 | 3.22E−04 | 0.15 | HSPA14 | 2.78 | 1.90E−02 | 2.6 |
| RPL31 | 16.17 | 6.39E−57 | 1.04 | ARL3 | 5.97 | 2.08E−08 | 0.57 | VPS51 | 3.99 | 3.30E−04 | 0.14 | HMCN2 | 2.78 | 1.90E−02 | 0.74 |
| HLA-A | 16.16 | 8.40E−57 | 0.83 | PGAM1 | 5.97 | 2.00E−08 | 0.45 | UBE2G1 | 3.99 | 3.22E−04 | 0.12 | LGALSL | 2.78 | 1.86E−02 | 0.72 |
| ATF6B | 16.13 | 1.34E−56 | 1.13 | FSTL1 | 5.97 | 2.06E−08 | 0.45 | NOMO2 | 3.98 | 3.46E−04 | 0.8 | IL16 | 2.78 | 1.87E−02 | 0.53 |
| ZNF90 | 16.11 | 1.71E−56 | 1.4 | EIF4H | 5.97 | 2.05E−08 | 0.33 | ILDR1 | 3.98 | 3.35E−04 | 0.68 | JMJD4 | 2.78 | 1.88E−02 | 0.42 |
| TMC4 | 16.06 | 3.85E−56 | 0.71 | GSTK1 | 5.97 | 2.00E−08 | 0.32 | MYO16-AS1 | 3.98 | 3.48E−04 | 0.67 | ARMC7 | 2.78 | 1.89E−02 | 0.3 |
| BCOR | 16.04 | 5.27E−56 | 1.37 | RNF126 | 5.97 | 2.05E−08 | 0.32 | COMMD6 | 3.98 | 3.40E−04 | 0.45 | PLPPR2 | 2.78 | 1.89E−02 | 0.29 |
| PRSS8 | 16 | 1.05E−55 | 1.24 | CASP8 | 5.97 | 2.08E−08 | 0.31 | SNRPD1 | 3.98 | 3.43E−04 | 0.24 | LINC00571 | 2.78 | 1.86E−02 | 0.11 |
| MGLL | 15.95 | 2.43E−55 | 0.7 | PEX14 | 5.97 | 2.03E−08 | 0.28 | ZNF263 | 3.98 | 3.38E−04 | 0.17 | CAPN2 | 2.78 | 1.90E−02 | 0.11 |
| PPP2R2B | 15.85 | 1.07E−54 | 2.83 | PACRG | 5.96 | 2.20E−08 | 0.64 | LSM2 | 3.97 | 3.58E−04 | 0.85 | DENND5B | 2.78 | 1.86E−02 | 0.1 |
| RPS11 | 15.82 | 1.81E−54 | 1.01 | MAP2K7 | 5.96 | 2.24E−08 | 0.33 | C8orf33 | 3.97 | 3.55E−04 | 0.84 | PUDP | 2.78 | 1.88E−02 | 0.06 |
| HOXB3 | 15.79 | 2.88E−54 | 1.21 | GCSH | 5.95 | 2.32E−08 | 2.15 | POLR2J | 3.97 | 3.50E−04 | 0.73 | FBXO46 | 2.78 | 1.90E−02 | 0.05 |
| PDXDC1 | 15.76 | 4.77E−54 | 0.54 | DSCAML1 | 5.95 | 2.34E−08 | 1.3 | DEGS1 | 3.97 | 3.57E−04 | 0.65 | ZNF335 | 2.78 | 1.91E−02 | 0.02 |
| TIMP2 | 15.7 | 1.09E−53 | 0.91 | STAG3 | 5.95 | 2.30E−08 | 1.05 | CCDC142 | 3.97 | 3.57E−04 | 0.49 | TROVE2 | 2.78 | 1.86E−02 | −0.01 |
| AIG1 | 15.68 | 1.44E−53 | 0.8 | POLR2K | 5.94 | 2.45E−08 | 1.23 | NEK10 | 3.97 | 3.60E−04 | 0.48 | CCZ1B | 2.78 | 1.88E−02 | −0.06 |
| PARP14 | 15.64 | 3.03E−53 | 0.74 | MYO16 | 5.94 | 2.41E−08 | 1.09 | EBNA1BP2 | 3.97 | 3.55E−04 | 0.47 | NR2E3 | 2.77 | 1.95E−02 | 1.59 |
| STAT1 | 15.63 | 3.19E−53 | 0.98 | TIMM50 | 5.94 | 2.48E−08 | 0.82 | RANBPI | 3.97 | 3.52E−04 | 0.41 | LAGE3 | 2.77 | 1.93E−02 | 1.26 |
| POLR2L | 15.58 | 7.70E−53 | 1.71 | KIZ-AS1 | 5.93 | 2.64E−08 | 1.1 | NBPF1 | 3.97 | 3.55E−04 | 0.33 | STAC | 2.77 | 1.93E−02 | 0.99 |
| LITAF | 15.54 | 1.26E−52 | 0.92 | ZNF439 | 5.93 | 2.56E−08 | 0.88 | PROCR | 3.97 | 3.48E−04 | 0.31 | ITGB2 | 2.77 | 1.93E−02 | 0.89 |
| RPL37A | 15.53 | 1.46E−52 | 0.98 | C19orf70 | 5.93 | 2.55E−08 | 0.72 | TXNDC9 | 3.97 | 3.54E−04 | 0.27 | TTC25 | 2.77 | 1.95E−02 | 0.83 |
| CFL1 | 15.44 | 6.15E−52 | 1.17 | TBCB | 5.93 | 2.67E−08 | 0.52 | TCHP | 3.97 | 3.59E−04 | 0.27 | SRP14-AS1 | 2.77 | 1.94E−02 | 0.78 |
| SAMD4A | 15.43 | 7.53E−52 | 0.73 | ADAP1 | 5.93 | 2.65E−08 | 0.47 | VWA2 | 3.97 | 3.48E−04 | 0.26 | MRPS2 | 2.77 | 1.92E−02 | 0.33 |
| CORO1C | 15.41 | 1.07E−51 | 0.83 | ACLY | 5.93 | 2.58E−08 | 0.38 | UBA6-AS1 | 3.97 | 3.57E−04 | 0.24 | STRN4 | 2.77 | 1.95E−02 | 0.24 |
| RPL10A | 15.33 | 3.60E−51 | 0.98 | CCDC92 | 5.93 | 2.66E−08 | 0.36 | CBWD1 | 3.97 | 3.49E−04 | 0.2 | MMAA | 2.77 | 1.92E−02 | 0.15 |
| RPL19 | 15.3 | 5.45E−51 | 1.16 | FRMD3 | 5.93 | 2.59E−08 | 0.34 | SPATA17 | 3.97 | 3.49E−04 | 0.17 | TOLLIP | 2.77 | 1.91E−02 | 0.05 |
| MID1 | 15.3 | 5.32E−51 | 0.73 | PIM3 | 5.93 | 2.56E−08 | 0.25 | FAM104A | 3.97 | 3.54E−04 | 0.17 | HOXA13 | 2.76 | 2.02E−02 | 1.87 |
| MET | 15.27 | 8.01E−51 | 0.56 | PCDH15 | 5.92 | 2.77E−08 | 0.97 | PLEKHG1 | 3.97 | 3.51E−04 | 0.16 | BLOC1S5-TXNDC5 | 2.76 | 2.02E−02 | 0.54 |
| DES | 15.22 | 1.76E−50 | 4.25 | ZNF213-AS1 | 5.92 | 2.81E−08 | 0.74 | HDHD3 | 3.97 | 3.60E−04 | 0.08 | QDPR | 2.76 | 1.97E−02 | 0.49 |
| MAPK8IP3 | 15.21 | 2.04E−50 | 0.71 | POLR2F | 5.92 | 2.79E−08 | 0.72 | ALOX12-AS1 | 3.97 | 3.55E−04 | 0.04 | MRPL23 | 2.76 | 1.99E−02 | 0.49 |
| TMPRSS4 | 15.21 | 2.07E−50 | 0.68 | ZNF512B | 5.92 | 2.77E−08 | 0.63 | KCNH1 | 3.96 | 3.71E−04 | 1.74 | C20orf96 | 2.76 | 1.97E−02 | 0.43 |
| SEC62 | 15.19 | 2.66E−50 | 0.85 | C8orf37-AS1 | 5.92 | 2.70E−08 | 0.62 | MTUS2 | 3.96 | 3.64E−04 | 1.12 | CEP76 | 2.76 | 1.98E−02 | 0.33 |
| RPS2 | 15.18 | 3.26E−50 | 0.93 | OSBP | 5.92 | 2.78E−08 | 0.29 | NPIPB11 | 3.96 | 3.67E−04 | 0.91 | TIRAP | 2.76 | 1.98E−02 | 0.11 |
| FLOT1 | 15.17 | 3.75E−50 | 0.88 | ODF2L | 5.92 | 2.69E−08 | 0.24 | PCSK5 | 3.96 | 3.66E−04 | 0.26 | ERBB2 | 2.76 | 1.98E−02 | 0.09 |
| SMIM22 | 15.16 | 4.39E−50 | 2.03 | ARMC2 | 5.91 | 2.92E−08 | 0.49 | ZSCAN5A | 3.96 | 3.68E−04 | 0.19 | CASP4 | 2.76 | 1.98E−02 | 0.06 |
| LRRFIP1 | 15.11 | 9.27E−50 | 0.43 | UBTF | 5.91 | 2.98E−08 | 0.35 | EAPP | 3.96 | 3.67E−04 | 0.16 | PGPEP1 | 2.76 | 1.98E−02 | 0 |
| RPL34 | 15.09 | 1.19E−49 | 1.16 | TPBG | 5.91 | 2.83E−08 | 0.33 | PROM2 | 3.96 | 3.64E−04 | 0.15 | C5orf15 | 2.76 | 2.00E−02 | 0 |
| ARMC9 | 15.09 | 1.20E−49 | 0.88 | PRPF40B | 5.9 | 3.18E−08 | 0.35 | TOB2 | 3.96 | 3.64E−04 | 0.14 | ZYG11B | 2.76 | 1.97E−02 | −0.01 |
| KCNK5 | 15.08 | 1.41E−49 | 1.42 | DPP6 | 5.9 | 3.13E−08 | 0.34 | ABCC8 | 3.95 | 3.85E−04 | 2 | DNAAF3 | 2.75 | 2.06E−02 | 1.53 |
| CHD4 | 15.06 | 1.90E−49 | 1.58 | LAMB1 | 5.9 | 3.10E−08 | 0.27 | C8orf34 | 3.95 | 3.76E−04 | 0.67 | GSG1L | 2.75 | 2.06E−02 | 1.52 |
| ARHGEF10 | 15.03 | 2.94E−49 | 2.56 | GREM1 | 5.89 | 3.23E−08 | 1.29 | RNF41 | 3.95 | 3.76E−04 | 0.38 | RAB36 | 2.75 | 2.04E−02 | 1.47 |
| SPATS2L | 15.03 | 3.11E−49 | 0.5 | SUMO3 | 5.89 | 3.36E−08 | 0.88 | SART1 | 3.95 | 3.86E−04 | 0.35 | MFSD5 | 2.75 | 2.06E−02 | 1.33 |
| ITGA3 | 15.01 | 4.02E−49 | 0.56 | PSMB3 | 5.89 | 3.28E−08 | 0.54 | SAMHDI | 3.95 | 3.89E−04 | 0.25 | TEX11 | 2.75 | 2.02E−02 | 0.98 |
| ZNF254 | 14.9 | 2.18E−48 | 0.86 | COX7A2 | 5.89 | 3.27E−08 | 0.52 | PLXDC2 | 3.95 | 3.85E−04 | 0.23 | HPSE2 | 2.75 | 2.03E−02 | 0.93 |
| TMBIM6 | 14.87 | 3.30E−48 | 0.91 | PROSER3 | 5.89 | 3.34E−08 | 0.5 | PLP2 | 3.95 | 3.88E−04 | 0.22 | LINC00893 | 2.75 | 2.06E−02 | 0.77 |
| ELAVL1 | 14.86 | 3.91E−48 | 0.91 | MFN2 | 5.89 | 3.28E−08 | 0.39 | LPCAT1 | 3.95 | 3.78E−04 | 0.17 | ACOXL | 2.75 | 2.06E−02 | 0.74 |
| RPS18 | 14.82 | 7.04E−48 | 1.26 | EIF3D | 5.89 | 3.36E−08 | 0.35 | CTDSP2 | 3.95 | 3.82E−04 | 0.11 | NTNG1 | 2.75 | 2.04E−02 | 0.63 |
| RPS15 | 14.82 | 6.73E−48 | 1.03 | TPD52 | 5.89 | 3.32E−08 | 0.26 | SCAMP3 | 3.95 | 3.84E−04 | 0.08 | MC1R | 2.75 | 2.06E−02 | 0.51 |
| ST3GAL3 | 14.81 | 8.18E−48 | 3.34 | GAN | 5.89 | 3.36E−08 | 0.26 | ST6GALNAC6 | 3.94 | 3.96E−04 | 3.86 | UROD | 2.75 | 2.04E−02 | 0.47 |
| SYNGR2 | 14.8 | 9.74E−48 | 1.23 | JMJD8 | 5.88 | 3.45E−08 | 0.93 | WDR63 | 3.94 | 4.04E−04 | 1.37 | GRPEL1 | 2.75 | 2.05E−02 | 0.41 |
| DGKG | 14.72 | 3.01E−47 | 1.58 | GYG1 | 5.88 | 3.49E−08 | 0.91 | GTF3C4 | 3.94 | 3.98E−04 | 0.43 | TRMT12 | 2.75 | 2.02E−02 | 0.39 |
| NRP2 | 14.72 | 2.98E−47 | 1.05 | NUBP2 | 5.88 | 3.48E−08 | 0.79 | ACTR6 | 3.94 | 3.90E−04 | 0.41 | EAF2 | 2.75 | 2.07E−02 | 0.39 |
| FGD6 | 14.72 | 2.96E−47 | 0.61 | PLBD1 | 5.88 | 3.46E−08 | 0.27 | OGFOD3 | 3.94 | 3.93E−04 | 0.36 | RTN2 | 2.75 | 2.05E−02 | 0.33 |
| MSLN | 14.71 | 3.42E−47 | 1.12 | ARPC2 | 5.88 | 3.51E−08 | 0.25 | MGAT1 | 3.94 | 3.99E−04 | 0.21 | CITED4 | 2.75 | 2.02E−02 | 0.32 |
| SH3TC1 | 14.69 | 4.74E−47 | 1 | PIR | 5.87 | 3.64E−08 | 0.77 | GSDMD | 3.94 | 4.03E−04 | 0.15 | MUTYH | 2.75 | 2.03E−02 | 0.28 |
| SLC39A11 | 14.68 | 4.92E−47 | 0.6 | ACO2 | 5.87 | 3.72E−08 | 0.25 | SUFU | 3.94 | 4.04E−04 | 0.14 | SOCS7 | 2.75 | 2.02E−02 | 0.06 |
| RPL35A | 14.67 | 6.02E−47 | 1.11 | CCDC167 | 5.86 | 3.96E−08 | 1.58 | CFI | 3.93 | 4.07E−04 | 1.8 | PRPSAP1 | 2.75 | 2.04E−02 | 0.05 |
| RPL8 | 14.66 | 6.67E−47 | 0.9 | TF | 5.86 | 3.97E−08 | 1.29 | MERTK | 3.93 | 4.14E−04 | 0.94 | TUBGCP2 | 2.75 | 2.03E−02 | 0.02 |
| FLNA | 14.61 | 1.42E−46 | 0.92 | ARSD | 5.86 | 3.84E−08 | 0.44 | SPEG | 3.93 | 4.07E−04 | 0.85 | MUS81 | 2.75 | 2.06E−02 | 0.02 |
| TRIM56 | 14.56 | 3.14E−46 | 0.75 | BRAP | 5.86 | 4.00E−08 | 0.36 | SCAMP5 | 3.93 | 4.15E−04 | 0.43 | CDC40 | 2.75 | 2.08E−02 | −0.07 |
| TIMP3 | 14.54 | 4.15E−46 | 1.71 | BIRC2 | 5.86 | 3.99E−08 | 0.3 | JMJD6 | 3.93 | 4.10E−04 | 0.37 | NAV2-AS3 | 2.74 | 2.11E−02 | 1.86 |
| SPNS2 | 14.5 | 7.08E−46 | 1.61 | CMTM4 | 5.86 | 3.88E−08 | 0.26 | RAB8A | 3.93 | 4.12E−04 | 0.33 | DUSP14 | 2.74 | 2.08E−02 | 0.82 |
| PXN | 14.5 | 6.80E−46 | 0.8 | SRPRA | 5.85 | 4.25E−08 | 0.69 | SRSF8 | 3.93 | 4.08E−04 | 0.31 | FAM104B | 2.74 | 2.13E−02 | 0.38 |
| CA3 | 14.44 | 1.79E−45 | 1.16 | GPR160 | 5.85 | 4.19E−08 | 0.51 | CDC27 | 3.93 | 4.2LE−04 | 0.16 | ARMCX6 | 2.74 | 2.13E−02 | 0.22 |
| DOK5 | 14.4 | 3.08E−45 | 2.36 | PLEKHG6 | 5.85 | 4.25E−08 | 0.47 | TRIB1 | 3.93 | 4.15E−04 | 0.14 | UBQLN2 | 2.74 | 2.10E−02 | 0.17 |
| RPS23 | 14.37 | 4.48E−45 | 1.1 | RAB20 | 5.85 | 4.05E−08 | 0.4 | HNRNPUL1 | 3.93 | 4.21E−04 | 0.12 | BSPRY | 2.74 | 2.10E−02 | 0.11 |
| RPL10 | 14.35 | 6.32E−45 | 0.61 | JADE2 | 5.85 | 4.08E−08 | 0.37 | FAM89B | 3.92 | 4.23E−04 | 0.78 | CDYL2 | 2.74 | 2.10E−02 | 0.08 |
| OOEP | 14.34 | 7.49E−45 | 1.18 | SNX8 | 5.85 | 4.09E−08 | 0.36 | E2F6 | 3.92 | 4.34E−04 | 0.36 | TRIM25 | 2.74 | 2.13E−02 | 0.08 |
| TMC6 | 14.31 | 1.10E−44 | 0.94 | NCBP2-AS2 | 5.84 | 4.48E−08 | 1.09 | C15orf40 | 3.92 | 4.28E−04 | 0.22 | POLR3A | 2.74 | 2.09E−02 | −0.04 |
| RNASE10 | 14.27 | 1.97E−44 | 1.79 | MANSC1 | 5.84 | 4.37E−08 | 0.89 | GOLGB1 | 3.92 | 4.23E−04 | 0.18 | SBDS | 2.74 | 2.10E−02 | −0.05 |
| SPG7 | 14.27 | 1.96E−44 | 0.62 | EPHX1 | 5.84 | 4.41E−08 | 0.76 | MYO9A | 3.92 | 4.39E−04 | 0.16 | OR2L13 | 2.73 | 2.14E−02 | 2.59 |
| BDNF-AS | 14.26 | 2.04E−44 | 1.11 | RNF8 | 5.84 | 4.28E−08 | 0.36 | THUMPD1 | 3.92 | 4.28E−04 | 0.14 | IGSF23 | 2.73 | 2.15E−02 | 1.53 |
| GFPT1 | 14.25 | 2.40E−44 | 0.66 | S100A13 | 5.84 | 4.46E−08 | 0.3 | KIAA0355 | 3.92 | 4.22E−04 | 0.13 | LSM10 | 2.73 | 2.14E−02 | 1.11 |
| BCAS3 | 14.24 | 2.76E−44 | 0.49 | MSN | 5.84 | 4.33E−08 | 0.22 | EIF2AK3 | 3.92 | 4.22E−04 | 0.09 | C1orf216 | 2.73 | 2.19E−02 | 0.96 |
| RHOC | 14.19 | 5.60E−44 | 1.23 | MS4A5 | 5.83 | 4.63E−08 | 2.13 | AC007389.3 | 3.91 | 4.47E−04 | 2.76 | C15orf39 | 2.73 | 2.16E−02 | 0.7 |
| IFI44 | 14.18 | 6.64E−44 | 1.09 | MDC1 | 5.83 | 4.55E−08 | 0.93 | DOHH | 3.91 | 4.47E−04 | 1.23 | CXCL17 | 2.73 | 2.17E−02 | 0.49 |
| EPHB2 | 14.09 | 2.50E−43 | 1.28 | GALM | 5.83 | 4.69E−08 | 0.56 | POLR3H | 3.91 | 4.54E−04 | 0.75 | GLDN | 2.73 | 2.16E−02 | 0.4 |
| CTSZ | 14.07 | 3.35E−43 | 1.25 | ZFP14 | 5.83 | 4.68E−08 | 0.38 | GPM6B | 3.91 | 4.54E−04 | 0.68 | GPC4 | 2.73 | 2.16E−02 | 0.26 |
| C16orf45 | 14.02 | 6.48E−43 | 0.7 | POLR2B | 5.83 | 4.75E−08 | 0.26 | MIEF1 | 3.91 | 4.55E−04 | 0.6 | EPHA10 | 2.73 | 2.14E−02 | 0.24 |
| TOMM7 | 14 | 8.56E−43 | 1.3 | CMTM3 | 5.82 | 4.79E−08 | 0.98 | KCNJ14 | 3.91 | 4.52E−04 | 0.52 | NKAP | 2.73 | 2.19E−02 | 0.22 |
| COL4A2 | 13.99 | 9.16E−43 | 1.59 | GTF2H5 | 5.82 | 4.86E−08 | 0.94 | ZFAND2B | 3.91 | 4.44E−04 | 0.3 | PAFAH2 | 2.73 | 2.16E−02 | 0.21 |
| MTIE | 13.94 | 1.85E−42 | 2.42 | MMP25-AS1 | 5.82 | 4.89E−08 | 0.88 | EPN1 | 3.91 | 4.47E−04 | 0.21 | PERP | 2.73 | 2.17E−02 | 0.15 |
| MYL6 | 13.93 | 2.27E−42 | 0.76 | BRMS1 | 5.82 | 5.02E−08 | 0.82 | YWHAE | 3.91 | 4.56E−04 | 0.16 | ANKRD40 | 2.73 | 2.19E−02 | 0.14 |
| IFITM3 | 13.91 | 2.71E−42 | 1.31 | MRPS10 | 5.82 | 4.97E−08 | 0.76 | MTM1 | 3.91 | 4.40E−04 | 0.15 | IFI27L1 | 2.73 | 2.16E−02 | 0.12 |
| CHKA | 13.91 | 2.78E−42 | 0.66 | HTATIP2 | 5.82 | 5.01E−08 | 0.49 | SEMA3B | 3.91 | 4.47E−04 | 0.14 | RAD23B | 2.73 | 2.16E−02 | 0.08 |
| AGRN | 13.9 | 3.23E−42 | 0.86 | KARS | 5.82 | 4.81E−08 | 0.4 | STARD7 | 3.91 | 4.52E−04 | 0.13 | BBS9 | 2.73 | 2.17E−02 | 0.07 |
| ILIRAP | 13.9 | 3.23E−42 | 0.69 | NAA38 | 5.82 | 4.81E−08 | 0.37 | TCTN3 | 3.91 | 4.56E−04 | 0.08 | SYNJ1 | 2.73 | 2.14E−02 | 0.04 |
| ONECUT2 | 13.89 | 4.03E−42 | 0.83 | BCL2L14 | 5.82 | 4.81E−08 | 0.32 | TOMM22 | 3.9 | 4.73E−04 | 0.94 | IGBP1 | 2.73 | 2.18E−02 | 0.01 |
| RPS25 | 13.87 | 5.26E−42 | 1.14 | CACNA2D3 | 5.81 | 5.25E−08 | 1.05 | MARCKSL1 | 3.9 | 4.62E−04 | 0.51 | FASTKD2 | 2.73 | 2.17E−02 | −0.01 |
| TAPBP | 13.87 | 4.85E−42 | 0.78 | FIGN | 5.81 | 5.08E−08 | 0.83 | ZNF770 | 3.9 | 4.65E−04 | 0.27 | C1orf43 | 2.73 | 2.18E−02 | −0.06 |
| DNASE1 | 13.87 | 4.94E−42 | 0.75 | PLXND1 | 5.81 | 5.15E−08 | 0.7 | HID1 | 3.9 | 4.62E−04 | 0.22 | FNDC4 | 2.72 | 2.20E−02 | 0.95 |
| DDR1 | 13.85 | 6.82E−42 | 1.02 | LRRC49 | 5.81 | 5.11E−08 | 0.66 | PDCD6 | 3.9 | 4.63E−04 | 0.14 | MUC6 | 2.72 | 2.20E−02 | 0.94 |
| CNKSR3 | 13.85 | 6.50E−42 | 0.89 | TXNRD2 | 5.81 | 5.27E−08 | 0.53 | RGS17 | 3.89 | 4.91E−04 | 1.1 | CLEC12A | 2.72 | 2.23E−02 | 0.72 |
| KIZ | 13.84 | 7.29E−42 | 1.05 | MCEE | 5.81 | 5.12E−08 | 0.45 | TMEM220-AS1 | 3.89 | 4.83E−04 | 0.75 | CLU | 2.72 | 2.24E−02 | 0.64 |
| ZNFX1 | 13.83 | 8.36E−42 | 0.79 | SZRD1 | 5.81 | 5.29E−08 | 0.44 | PFDN4 | 3.89 | 4.91E−04 | 0.7 | GGCT | 2.72 | 2.22E−02 | 0.57 |
| OR2A1-AS1 | 13.82 | 1.02E−41 | 3.67 | CMSS1 | 5.81 | 5.16E−08 | 0.25 | KLF16 | 3.89 | 4.78E−04 | 0.65 | DDX11-AS1 | 2.72 | 2.22E−02 | 0.56 |
| PRSS23 | 13.79 | 1.54E−41 | 1.54 | ERCC5 | 5.8 | 5.56E−08 | 2.7 | IKBKG | 3.89 | 4.91E−04 | 0.43 | C2orf74 | 2.72 | 2.24E−02 | 0.41 |
| CALR | 13.78 | 1.72E−41 | 1.18 | UBXN1 | 5.8 | 5.55E−08 | 0.51 | PPP2R5B | 3.89 | 4.87E−04 | 0.35 | APBA3 | 2.72 | 2.20E−02 | 0.39 |
| HSPB1 | 13.76 | 2.31E−41 | 0.73 | SPINT1 | 5.8 | 5.41E−08 | 0.29 | CRACR2B | 3.89 | 4.84E−04 | 0.27 | NOB1 | 2.72 | 2.23E−02 | 0.35 |
| MYH14 | 13.73 | 3.44E−41 | 0.55 | ZNF587 | 5.8 | 5.33E−08 | 0.26 | EARS2 | 3.89 | 4.88E−04 | 0.25 | LINC00970 | 2.72 | 2.22E−02 | 0.32 |
| ISG15 | 13.72 | 4.02E−41 | 2.15 | TRHDE | 5.79 | 5.82E−08 | 0.78 | ATP13A1 | 3.89 | 4.79E−04 | 0.23 | TMEM30B | 2.72 | 2.24E−02 | 0.32 |
| CHPF | 13.72 | 4.07E−41 | 2.07 | TPRA1 | 5.79 | 5.85E−08 | 0.74 | RNF146 | 3.89 | 4.79E−04 | 0.13 | ESRRG | 2.72 | 2.25E−02 | 0.21 |
| RPS20 | 13.72 | 3.59E−41 | 0.74 | CDC42EP3 | 5.78 | 6.00E−08 | 0.79 | NDUFV1 | 3.89 | 4.83E−04 | 0.11 | SLC8A1 | 2.72 | 2.23E−02 | 0.19 |
| RPL6 | 13.68 | 6.49E−41 | 0.98 | RBKS | 5.78 | 6.18E−08 | 0.79 | LGMN | 3.89 | 4.80E−04 | 0.1 | STK11IP | 2.72 | 2.23E−02 | 0.14 |
| CLDN1 | 13.59 | 2.23E−40 | 0.79 | SOAT1 | 5.78 | 6.08E−08 | 0.33 | FKBP15 | 3.89 | 4.79E−04 | 0.09 | UMPS | 2.72 | 2.21E−02 | 0.12 |
| SLC35A3 | 13.58 | 2.57E−40 | 3.06 | KPNB1 | 5.78 | 6.14E−08 | 0.26 | CSMD2 | 3.88 | 4.97E−04 | 1.03 | HIVEP2 | 2.72 | 2.25E−02 | 0.11 |
| SIK3 | 13.56 | 3.41E−40 | 0.47 | CDIPT | 5.77 | 6.33E−08 | 0.96 | EMC4 | 3.88 | 5.00E−04 | 0.79 | PDPK1 | 2.72 | 2.25E−02 | 0.09 |
| ZFAS1 | 13.51 | 6.55E−40 | 0.75 | GABARAPL2 | 5.77 | 6.67E−08 | 0.74 | RSL1D1 | 3.88 | 5.09E−04 | 0.28 | ZNF142 | 2.72 | 2.21E−02 | 0.06 |
| MYL9 | 13.48 | 9.37E−40 | 1.57 | SFN | 5.77 | 6.68E−08 | 0.56 | FLII | 3.88 | 4.99E−04 | 0.25 | GMPS | 2.72 | 2.22E−02 | 0.04 |
| LGALS3BP | 13.48 | 9.27E−40 | 1.27 | MRFAP1 | 5.77 | 6.53E−08 | 0.49 | GAR1 | 3.88 | 5.09E−04 | 0.23 | FAM122C | 2.72 | 2.25E−02 | 0.03 |
| FOXP2 | 13.46 | 1.33E−39 | 0.8 | GRAMDIC | 5.77 | 6.36E−08 | 0.43 | TSEN54 | 3.88 | 5.06E−04 | 0.2 | RPS6KB2 | 2.72 | 2.23E−02 | 0.02 |
| TNFRSF12A | 13.45 | 1.40E−39 | 1.38 | C8orf59 | 5.77 | 6.36E−08 | 0.32 | SNX4 | 3.88 | 5.09E−04 | 0.16 | MAP3K7 | 2.72 | 2.20E−02 | 0 |
| PDE4D | 13.44 | 1.74E−39 | 0.47 | EIF3M | 5.77 | 6.49E−08 | 0.26 | HMGXB3 | 3.88 | 4.97E−04 | 0.13 | ARL6IP5 | 2.72 | 2.21E−02 | 0 |
| STAT2 | 13.4 | 2.94E−39 | 0.82 | CYC1 | 5.76 | 6.81E−08 | 0.59 | ZNF804B | 3.87 | 5.30E−04 | 1.47 | RELA | 2.72 | 2.20E−02 | −0.03 |
| RARRES3 | 13.39 | 3.24E−39 | 2.14 | LZTR1 | 5.76 | 6.93E−08 | 0.55 | RSL24D1 | 3.87 | 5.13E−04 | 0.46 | RASA4B | 2.71 | 2.31E−02 | 3.48 |
| RPS8 | 13.38 | 3.72E−39 | 0.86 | FYN | 5.76 | 6.96E−08 | 0.45 | L3MBTL2 | 3.87 | 5.28E−04 | 0.36 | ZNF667-AS1 | 2.71 | 2.31E−02 | 1.51 |
| CCDC40 | 13.33 | 7.15E−39 | 1.47 | NCBP2 | 5.76 | 6.99E−08 | 0.44 | BRK1 | 3.87 | 5.26E−04 | 0.18 | C6 | 2.71 | 2.26E−02 | 1.46 |
| COPG1 | 13.33 | 7.43E−39 | 0.99 | RNF34 | 5.76 | 6.74E−08 | 0.39 | LETM1 | 3.87 | 5.13E−04 | 0.13 | SLC24A2 | 2.71 | 2.28E−02 | 1.31 |
| MYEOV | 13.33 | 7.43E−39 | 0.59 | ADCY9 | 5.76 | 6.93E−08 | 0.35 | IER3 | 3.87 | 5.30E−04 | 0.13 | WDR88 | 2.71 | 2.29E−02 | 0.86 |
| RUVBL1 | 13.32 | 7.92E−39 | 0.93 | SFSWAP | 5.76 | 6.90E−08 | 0.29 | SLC25A22 | 3.86 | 5.47E−04 | 0.82 | NPM3 | 2.71 | 2.28E−02 | 0.66 |
| AMPD3 | 13.28 | 1.41E−38 | 1.39 | NAA25 | 5.76 | 6.82E−08 | 0.27 | KLRD1 | 3.86 | 5.49E−04 | 0.69 | ZNF30 | 2.71 | 2.28E−02 | 0.55 |
| NAPA | 13.28 | 1.44E−38 | 1.08 | PLCG2 | 5.76 | 6.90E−08 | 0.21 | TTLL1 | 3.86 | 5.52E−04 | 0.65 | DDX41 | 2.71 | 2.30E−02 | 0.39 |
| NME3 | 13.27 | 1.50E−38 | 0.76 | VWDE | 5.75 | 7.31E−08 | 0.6 | BST2 | 3.86 | 5.50E−04 | 0.55 | PDZRN3 | 2.71 | 2.31E−02 | 0.33 |
| PAPSS2 | 13.23 | 2.84E−38 | 1.42 | LEMD2 | 5.75 | 7.18E−08 | 0.41 | BBOF1 | 3.86 | 5.33E−04 | 0.52 | ZNF426 | 2.71 | 2.26E−02 | 0.23 |
| RBCK1 | 13.23 | 2.51E−38 | 0.82 | CLIP1 | 5.75 | 7.22E−08 | 0.25 | WASF1 | 3.86 | 5.35E−04 | 0.42 | SLC39A13 | 2.71 | 2.27E−02 | 0.23 |
| XPO5 | 13.22 | 3.09E−38 | 0.8 | RGN | 5.74 | 7.88E−08 | 2.49 | CCZ1 | 3.86 | 5.39E−04 | 0.15 | SPAG7 | 2.71 | 2.27E−02 | 0.17 |
| GPR75-ASB3 | 13.2 | 4.08E−38 | 1.15 | ANAPC13 | 5.74 | 7.90E−08 | 1.36 | PEX3 | 3.86 | 5.40E−04 | 0.14 | SMIM14 | 2.71 | 2.31E−02 | 0.07 |
| BCAP31 | 13.2 | 3.94E−38 | 1.06 | SPDYA | 5.74 | 7.84E−08 | 0.89 | SPATA7 | 3.86 | 5.52E−04 | 0.12 | RNLS | 2.71 | 2.31E−02 | 0.07 |
| RPS28 | 13.19 | 4.41E−38 | 1.41 | AMOTL2 | 5.74 | 7.88E−08 | 0.83 | NUDT5 | 3.86 | 5.43E−04 | 0.08 | BCL3 | 2.71 | 2.27E−02 | 0.05 |
| ASS1 | 13.19 | 4.45E−38 | 1.29 | TMEM106B | 5.74 | 7.66E−08 | 0.41 | TTC23 | 3.86 | 5.39E−04 | 0.07 | VPS4B | 2.71 | 2.28E−02 | 0.03 |
| ZNF529-AS1 | 13.15 | 8.01E−38 | 1.25 | ATP6V0E1 | 5.74 | 7.92E−08 | 0.27 | MRPS18A | 3.85 | 5.60E−04 | 0.74 | APPL2 | 2.71 | 2.30E−02 | 0.03 |
| RERG | 13.15 | 7.97E−38 | 1.1 | CES4A | 5.73 | 8.17E−08 | 1.34 | TMEM167B | 3.85 | 5.59E−04 | 0.71 | ARFGAP3 | 2.71 | 2.30E−02 | 0.03 |
| TPCN1 | 13.11 | 1.32E−37 | 0.72 | TAX1BP3 | 5.73 | 7.92E−08 | 0.99 | ZMAT2 | 3.85 | 5.60E−04 | 0.43 | MTPN | 2.71 | 2.28E−02 | 0.02 |
| CRHR1 | 13.1 | 1.35E−37 | 2.84 | APMAP | 5.73 | 7.96E−08 | 0.64 | PIH1D1 | 3.85 | 5.69E−04 | 0.31 | RNF14 | 2.71 | 2.31E−02 | 0.02 |
| RPN1 | 13.1 | 1.42E−37 | 1.16 | ARHGEF17 | 5.73 | 7.96E−08 | 0.55 | RARA | 3.85 | 5.55E−04 | 0.27 | ZNF146 | 2.71 | 2.27E−02 | −0.02 |
| FAU | 13.04 | 3.28E−37 | 1.13 | TRAPPC3 | 5.73 | 8.31E−08 | 0.54 | AK9 | 3.85 | 5.57E−04 | 0.27 | ST6GALNAC5 | 2.7 | 2.32E−02 | 0.88 |
| SPSB3 | 13.04 | 3.01E−37 | 0.82 | HSPA9 | 5.73 | 7.92E−08 | 0.23 | TTC26 | 3.85 | 5.74E−04 | 0.27 | ZSCAN16-AS1 | 2.7 | 2.32E−02 | 0.84 |
| MYO3B | 12.99 | 5.62E−37 | 2.73 | FOXO3 | 5.73 | 7.94E−08 | 0.23 | PTDSS2 | 3.85 | 5.63E−04 | 0.21 | HIST3H2A | 2.7 | 2.37E−02 | 0.8 |
| RPS6KA2 | 12.94 | 1.10E−36 | 0.71 | TLN2 | 5.72 | 8.79E−08 | 0.33 | SRGAP2C | 3.85 | 5.59E−04 | 0.17 | B3GALT4 | 2.7 | 2.31E−02 | 0.71 |
| AP3S1 | 12.93 | 1.37E−36 | 0.68 | RHOBTB3 | 5.72 | 8.83E−08 | 0.27 | RPS6KA1 | 3.85 | 5.73E−04 | 0.12 | ZCWPW1 | 2.7 | 2.33E−02 | 0.46 |
| UBC | 12.91 | 1.67E−36 | 0.48 | KDM5C | 5.72 | 8.62E−08 | 0.26 | ARHGDIB | 3.84 | 5.80E−04 | 0.59 | SLF1 | 2.7 | 2.34E−02 | 0.13 |
| MT-CO3 | 12.89 | 2.03E−36 | 0.61 | VPS35 | 5.72 | 8.80E−08 | 0.26 | POU2F2 | 3.84 | 5.76E−04 | 0.41 | RHOXF1-AS1 | 2.7 | 2.34E−02 | 0.13 |
| PTPRS | 12.88 | 2.30E−36 | 1.23 | NXN | 5.72 | 8.62E−08 | 0.22 | BACE1 | 3.84 | 5.97E−04 | 0.27 | PUF60 | 2.7 | 2.36E−02 | 0.12 |
| FIRRE | 12.88 | 2.54E−36 | 1 | CAMKK1 | 5.71 | 9.00E−08 | 1.06 | FBXO7 | 3.84 | 5.90E−04 | 0.06 | TIAM2 | 2.7 | 2.32E−02 | 0.09 |
| OARD1 | 12.87 | 2.91E−36 | 1.22 | COX17 | 5.71 | 9.23E−08 | 1.04 | KIF22 | 3.83 | 6.10E−04 | 0.5 | NHS | 2.7 | 2.33E−02 | 0.07 |
| TTC7A | 12.87 | 2.64E−36 | 0.68 | CYP19A1 | 5.71 | 9.34E−08 | 0.94 | GRB14 | 3.83 | 6.04E−04 | 0.47 | ALDH3A2 | 2.7 | 2.34E−02 | 0.01 |
| HSPBAP1 | 12.86 | 3.08E−36 | 1.07 | CYB5R1 | 5.71 | 9.31E−08 | 0.72 | AK8 | 3.83 | 6.13E−04 | 0.33 | COPS4 | 2.7 | 2.34E−02 | −0.02 |
| CDK14 | 12.85 | 3.50E−36 | 0.56 | RUVBL2 | 5.71 | 9.35E−08 | 0.72 | COPS7A | 3.83 | 6.13E−04 | 0.32 | LHFPL5 | 2.69 | 2.38E−02 | 1.12 |
| CAST | 12.85 | 3.69E−36 | 0.49 | NR2F2 | 5.71 | 9.24E−08 | 0.44 | AGBL5 | 3.83 | 6.04E−04 | 0.25 | AC009299.3 | 2.69 | 2.39E−02 | 1 |
| RPL12 | 12.83 | 4.63E−36 | 0.86 | TNXB | 5.7 | 9.78E−08 | 1.48 | HACE1 | 3.83 | 6.11E−04 | 0.19 | PDK2 | 2.69 | 2.40E−02 | 0.86 |
| CYP7B1 | 12.81 | 6.12E−36 | 1.15 | ZNF593 | 5.7 | 9.81E−08 | 1.17 | MCTS1 | 3.83 | 6.19E−04 | 0.16 | POLR2M | 2.69 | 2.39E−02 | 0.53 |
| SEC24D | 12.8 | 6.88E−36 | 0.69 | BCAS4 | 5.7 | 9.82E−08 | 0.55 | PLEKHG3 | 3.83 | 6.01E−04 | 0.15 | MRPL34 | 2.69 | 2.42E−02 | 0.37 |
| RABAC1 | 12.78 | 9.03E−36 | 1.56 | RRP7A | 5.7 | 9.76E−08 | 0.4 | SGSM2 | 3.83 | 6.15E−04 | 0.15 | LCAT | 2.69 | 2.40E−02 | 0.31 |
| TAGLN | 12.77 | 1.01E−35 | 1.36 | MYL12B | 5.7 | 9.50E−08 | 0.37 | AIMP1 | 3.83 | 6.07E−04 | 0.12 | SIRPA | 2.69 | 2.39E−02 | 0.29 |
| MAMDC2 | 12.77 | 9.62E−36 | 1.04 | SCAMP2 | 5.7 | 9.46E−08 | 0.33 | CRK | 3.83 | 6.19E−04 | 0.11 | ZDHHC18 | 2.69 | 2.41E−02 | 0.25 |
| BAIAP2L2 | 12.76 | 1.13E−35 | 0.74 | GLO1 | 5.69 | 1.01E−07 | 0.58 | SRM | 3.82 | 6.42E−04 | 0.64 | TMEM138 | 2.69 | 2.44E−02 | 0.24 |
| ANXA8L1 | 12.75 | 1.19E−35 | 2.81 | MECR | 5.69 | 1.00E−07 | 0.56 | SURF1 | 3.82 | 6.38E−04 | 0.62 | ZKSCAN8 | 2.69 | 2.39E−02 | 0.18 |
| MMP3 | 12.74 | 1.36E−35 | 1.73 | ARL17B | 5.69 | 1.01E−07 | 0.26 | ZFAND2A | 3.82 | 6.43E−04 | 0.52 | MRPS23 | 2.69 | 2.40E−02 | 0.15 |
| RSPH3 | 12.71 | 2.11E−35 | 0.93 | MYO1E | 5.69 | 1.03E−07 | 0.19 | PDCL | 3.82 | 6.41E−04 | 0.4 | ZNF827 | 2.69 | 2.39E−02 | 0.07 |
| SERPINE1 | 12.7 | 2.50E−35 | 1.12 | MFGE8 | 5.68 | 1.10E−07 | 0.83 | HDAC10 | 3.82 | 6.21E−04 | 0.32 | ACTR1B | 2.69 | 2.43E−02 | −0.05 |
| PLA2G16 | 12.69 | 2.85E−35 | 0.74 | CACNB4 | 5.68 | 1.07E−07 | 0.77 | MXRA5 | 3.82 | 6.42E−04 | 0.28 | MDGA2 | 2.68 | 2.44E−02 | 1.18 |
| EEF1A1 | 12.68 | 3.11E−35 | 0.67 | SCARB1 | 5.68 | 1.07E−07 | 0.57 | TIGAR | 3.82 | 6.30E−04 | 0.24 | AC087501.1 | 2.68 | 2.46E−02 | 1.14 |
| GPC1 | 12.66 | 3.87E−35 | 1.25 | TCEA3 | 5.68 | 1.05E−07 | 0.54 | STK33 | 3.82 | 6.32E−04 | 0.21 | CFAP61 | 2.68 | 2.49E−02 | 0.85 |
| UBA52 | 12.65 | 4.31E−35 | 1.09 | RALGDS | 5.68 | 1.08E−07 | 0.33 | PLEKHA4 | 3.81 | 6.51E−04 | 0.37 | COL14A1 | 2.68 | 2.45E−02 | 0.66 |
| NOL4L | 12.63 | 6.04E−35 | 0.87 | ARHGAP29 | 5.68 | 1.06E−07 | 0.3 | ESYT1 | 3.81 | 6.49E−04 | 0.36 | ARHGAP20 | 2.68 | 2.45E−02 | 0.58 |
| LINC00511 | 12.62 | 6.44E−35 | 0.33 | PTS | 5.68 | 1.09E−07 | 0.3 | RCC2 | 3.81 | 6.60E−04 | 0.16 | BORCS8-MEF2B | 2.68 | 2.48E−02 | 0.5 |
| ARPC1B | 12.61 | 7.14E−35 | 0.97 | ZRANB3 | 5.68 | 1.07E−07 | 0.26 | NAPG | 3.81 | 6.63E−04 | 0.07 | GNPDA1 | 2.68 | 2.47E−02 | 0.28 |
| TEX41 | 12.59 | 9.21E−35 | 2.35 | CLCN7 | 5.68 | 1.06E−07 | 0.24 | HOTTIP | 3.8 | 6.82E−04 | 2.3 | DEF8 | 2.68 | 2.48E−02 | 0.24 |
| CTIF | 12.57 | 1.27E−34 | 0.96 | SAP30BP | 5.68 | 1.08E−07 | 0.22 | LRRC37A | 3.8 | 6.88E−04 | 0.68 | RBFOX2 | 2.68 | 2.47E−02 | 0.11 |
| RAMP | 12.52 | 2.34E−34 | 1.28 | ORMDL3 | 5.67 | 1.16E−07 | 1.2 | SLC35A2 | 3.8 | 6.93E−04 | 0.54 | SYPL1 | 2.68 | 2.45E−02 | 0.1 |
| TNFSF10 | 12.49 | 3.32E−34 | 1.07 | BTBD19 | 5.67 | 1.16E−07 | 0.6 | LINC00271 | 3.8 | 6.89E−04 | 0.49 | NCSTN | 2.68 | 2.46E−02 | 0.1 |
| CLSTN3 | 12.49 | 3.27E−34 | 1.02 | TEC | 5.67 | 1.11E−07 | 0.4 | RBFOX1 | 3.8 | 6.90E−04 | 0.45 | TMEM125 | 2.68 | 2.48E−02 | 0.08 |
| ZNF609 | 12.48 | 3.56E−34 | 0.44 | TMEM219 | 5.67 | 1.13E−07 | 0.31 | PUS1 | 3.8 | 6.79E−04 | 0.34 | CBX1 | 2.68 | 2.45E−02 | 0.07 |
| SPDYE5 | 12.46 | 4.77E−34 | 1.25 | CLIP2 | 5.67 | 1.14E−07 | 0.25 | FBXO16 | 3.8 | 6.84E−04 | 0.31 | UBA2 | 2.68 | 2.46E−02 | 0.06 |
| SPATS2 | 12.44 | 6.37E−34 | 0.62 | CRLF3 | 5.67 | 1.11E−07 | 0.24 | NDUFAB1 | 3.8 | 6.92E−04 | 0.3 | FERMT2 | 2.68 | 2.47E−02 | 0.06 |
| SVIL | 12.44 | 6.24E−34 | 0.49 | MRGBP | 5.66 | 1.17E−07 | 0.68 | UBAC1 | 3.8 | 6.95E−04 | 0.28 | GLRX | 2.68 | 2.45E−02 | 0.04 |
| ABLIM1 | 12.43 | 6.65E−34 | 0.53 | BIK | 5.66 | 1.18E−07 | 0.66 | ALKBH5 | 3.8 | 6.86E−04 | 0.27 | B3GNT5 | 2.68 | 2.47E−02 | 0.04 |
| TPM4 | 12.41 | 8.39E−34 | 0.55 | KMT5C | 5.66 | 1.23E−07 | 0.63 | NMI | 3.8 | 6.81E−04 | 0.23 | GABPB1 | 2.68 | 2.47E−02 | 0.04 |
| RPS12 | 12.39 | 1.18E−33 | 0.97 | NLRP1 | 5.66 | 1.17E−07 | 0.31 | TIMM23B | 3.8 | 6.89E−04 | 0.17 | MSANTD2 | 2.68 | 2.48E−02 | 0.04 |
| SPAG1 | 12.39 | 1.10E−33 | 0.89 | HNRNPA1 | 5.66 | 1.21E−07 | 0.27 | PLCH1 | 3.8 | 6.73E−04 | 0.16 | PPP6C | 2.68 | 2.48E−02 | 0.02 |
| KCNQ5 | 12.38 | 1.20E−33 | 1.72 | SMARCE1 | 5.65 | 1.28E−07 | 2.95 | HMG20B | 3.8 | 6.80E−04 | 0.13 | MXD4 | 2.68 | 2.46E−02 | 0 |
| MT-ND1 | 12.34 | 2.04E−33 | 0.82 | IMP3 | 5.65 | 1.28E−07 | 0.98 | WBP11 | 3.8 | 6.85E−04 | 0.11 | ZBTB11 | 2.68 | 2.47E−02 | 0 |
| TTLL7 | 12.31 | 2.80E−33 | 1.85 | ACOT7 | 5.65 | 1.28E−07 | 0.63 | PPP5D1 | 3.79 | 7.14E−04 | 0.58 | ALG14 | 2.68 | 2.48E−02 | −0.04 |
| RPS14 | 12.29 | 3.81E−33 | 0.85 | ING4 | 5.65 | 1.24E−07 | 0.34 | MAPRE3 | 3.79 | 7.00E−04 | 0.32 | RNF5 | 2.67 | 2.57E−02 | 1.06 |
| GAPDH | 12.27 | 4.75E−33 | 0.72 | ZNF248 | 5.65 | 1.25E−07 | 0.3 | MISP | 3.79 | 7.22E−04 | 0.24 | PTGES | 2.67 | 2.56E−02 | 0.82 |
| ZNF432 | 12.25 | 6.40E−33 | 0.85 | FAM126B | 5.65 | 1.25E−07 | 0.27 | MRPS16 | 3.79 | 7.17E−04 | 0.21 | GADD45G | 2.67 | 2.55E−02 | 0.78 |
| IGFBP4 | 12.24 | 7.13E−33 | 1.04 | FAM3B | 5.64 | 1.38E−07 | 0.73 | RMND1 | 3.79 | 7.14E−04 | 0.18 | GNAL | 2.67 | 2.57E−02 | 0.15 |
| TUBB | 12.23 | 7.51E−33 | 1.22 | MRPL14 | 5.64 | 1.38E−07 | 0.72 | ITGB8 | 3.79 | 7.14E−04 | 0.17 | TMEM161A | 2.67 | 2.55E−02 | 0.13 |
| TXNRD1 | 12.23 | 7.83E−33 | 0.64 | FAAP20 | 5.64 | 1.37E−07 | 0.5 | RBMS2 | 3.79 | 6.99E−04 | 0.14 | B3GAT3 | 2.67 | 2.52E−02 | 0.08 |
| EZR | 12.23 | 7.92E−33 | 0.39 | PITPNM2 | 5.64 | 1.38E−07 | 0.5 | NACC2 | 3.79 | 6.95E−04 | 0.13 | CYP20A1 | 2.67 | 2.52E−02 | 0.02 |
| SNRPD2 | 12.21 | 1.05E−32 | 1.7 | OS9 | 5.64 | 1.36E−07 | 0.32 | POLR2G | 3.78 | 7.42E−04 | 0.69 | DBN1 | 2.67 | 2.52E−02 | 0 |
| IGFBP7 | 12.21 | 9.94E−33 | 0.85 | ANXA7 | 5.64 | 1.37E−07 | 0.21 | ANXA6 | 3.78 | 7.47E−04 | 0.4 | PIGT | 2.67 | 2.54E−02 | −0.03 |
| H2AFJ | 12.21 | 1.03E−32 | 0.83 | TSPAN3 | 5.63 | 1.42E−07 | 0.37 | GLCCI1 | 3.78 | 7.47E−04 | 0.22 | PI3 | 2.66 | 2.61E−02 | 1.66 |
| RORA | 12.21 | 1.02E−32 | 0.51 | ARMCX4 | 5.63 | 1.42E−07 | 0.31 | ETS1 | 3.78 | 7.22E−04 | 0.18 | JPH2 | 2.66 | 2.63E−02 | 0.9 |
| IL10RB | 12.14 | 2.27E−32 | 2.51 | SLC16A5 | 5.63 | 1.38E−07 | 0.29 | COQ10B | 3.78 | 7.38E−04 | 0.14 | CPLX1 | 2.66 | 2.60E−02 | 0.72 |
| RAB31 | 12.13 | 2.54E−32 | 0.99 | HCG27 | 5.62 | 1.47E−07 | 1.55 | IVD | 3.78 | 7.46E−04 | 0.14 | WDR92 | 2.66 | 2.59E−02 | 0.63 |
| DCBLD2 | 12.09 | 4.05E−32 | 0.54 | CCDC124 | 5.62 | 1.54E−07 | 0.78 | EIF3F | 3.78 | 7.29E−04 | 0.1 | LINC00472 | 2.66 | 2.60E−02 | 0.59 |
| LINC01481 | 12.08 | 4.59E−32 | 3.01 | MFSD1 | 5.62 | 1.54E−07 | 0.49 | GTPBP1 | 3.78 | 7.37E−04 | 0.03 | TRIM69 | 2.66 | 2.62E−02 | 0.57 |
| RIN2 | 12.07 | 5.65E−32 | 0.54 | RABEPK | 5.62 | 1.49E−07 | 0.48 | SLC5A6 | 3.77 | 7.59E−04 | 0.56 | CENPA | 2.66 | 2.61E−02 | 0.45 |
| SCRN1 | 12.05 | 6.60E−32 | 0.79 | LINC00843 | 5.62 | 1.54E−07 | 0.42 | LINC00276 | 3.77 | 7.74E−04 | 0.49 | PARP2 | 2.66 | 2.60E−02 | 0.43 |
| URM1 | 12.04 | 7.54E−32 | 0.93 | PLS3 | 5.62 | 1.48E−07 | 0.27 | C4BPB | 3.77 | 7.77E−04 | 0.41 | ZNF205 | 2.66 | 2.62E−02 | 0.37 |
| B3GALT5 | 12.03 | 8.96E−32 | 0.75 | TRAP1 | 5.62 | 1.47E−07 | 0.18 | RSPH1 | 3.77 | 7.58E−04 | 0.34 | MFAP1 | 2.66 | 2.61E−02 | 0.16 |
| SEC61A1 | 11.98 | 1.57E−31 | 0.73 | TMEM217 | 5.61 | 1.56E−07 | 1.15 | ZCRB1 | 3.77 | 7.75E−04 | 0.25 | PPP1R3B | 2.66 | 2.58E−02 | 0.14 |
| USP31 | 11.97 | 1.87E−31 | 0.8 | ARL1 | 5.61 | 1.57E−07 | 0.72 | SCAMP4 | 3.77 | 7.77E−04 | 0.21 | TMEM94 | 2.66 | 2.63E−02 | 0.06 |
| SUPT6H | 11.96 | 2.03E−31 | 0.66 | GPR132 | 5.61 | 1.63E−07 | 0.6 | ATF3 | 3.77 | 7.61E−04 | 0.16 | STK36 | 2.66 | 2.62E−02 | 0.02 |
| ATXN2 | 11.96 | 2.09E−31 | 0.46 | CERK | 5.61 | 1.61E−07 | 0.55 | ZC3H15 | 3.77 | 7.58E−04 | 0.1 | HEXB | 2.66 | 2.61E−02 | −0.01 |
| COMMD7 | 11.95 | 2.26E−31 | 4.12 | PROS1 | 5.61 | 1.56E−07 | 0.43 | BZW1 | 3.77 | 7.53E−04 | 0.09 | CRYZ | 2.66 | 2.61E−02 | −0.01 |
| SLC33A1 | 11.93 | 2.79E−31 | 3.44 | APOL1 | 5.61 | 1.61E−07 | 0.27 | CD9 | 3.77 | 7.64E−04 | 0.09 | AC073218.1 | 2.65 | 2.69E−02 | 3.08 |
| HERC6 | 11.93 | 2.90E−31 | 1.07 | RGS12 | 5.61 | 1.56E−07 | 0.22 | TSPAN9 | 3.77 | 7.52E−04 | 0.07 | KCNH3 | 2.65 | 2.70E−02 | 1.71 |
| SLC7A5 | 11.92 | 3.15E−31 | 1.92 | LINC00884 | 5.6 | 1.68E−07 | 1.05 | CSNK1G1 | 3.77 | 7.70E−04 | 0.07 | TTLL6 | 2.65 | 2.65E−02 | 1.19 |
| RPL18 | 11.92 | 3.26E−31 | 0.9 | ELOF1 | 5.6 | 1.72E−07 | 0.88 | MRPL17 | 3.76 | 8.04E−04 | 0.95 | CA11 | 2.65 | 2.69E−02 | 0.98 |
| PML | 11.92 | 3.42E−31 | 0.76 | ITGA5 | 5.6 | 1.66E−07 | 0.87 | CNGA1 | 3.76 | 7.84E−04 | 0.65 | RCAN2 | 2.65 | 2.67E−02 | 0.62 |
| MIR99AHG | 11.91 | 3.83E−31 | 1.26 | APOL6 | 5.6 | 1.71E−07 | 0.54 | TMEM234 | 3.76 | 7.93E−04 | 0.48 | PPP1R3F | 2.65 | 2.68E−02 | 0.61 |
| AC072062.1 | 11.88 | 4.95E−31 | 2.38 | BIN1 | 5.6 | 1.70E−07 | 0.4 | DHRS4-AS1 | 3.76 | 7.79E−04 | 0.45 | C3orf67-AS1 | 2.65 | 2.69E−02 | 0.61 |
| SYTL3 | 11.88 | 5.34E−31 | 1.11 | GNB2 | 5.6 | 1.70E−07 | 0.36 | PPP1R14B | 3.76 | 7.79E−04 | 0.3 | ZNF473 | 2.65 | 2.71E−02 | 0.5 |
| NTN4 | 11.88 | 5.03E−31 | 0.65 | TTBK2 | 5.6 | 1.65E−07 | 0.29 | KCTD2 | 3.76 | 7.87E−04 | 0.23 | HTRA1 | 2.65 | 2.71E−02 | 0.48 |
| HSP90B1 | 11.85 | 7.68E−31 | 0.81 | GGA2 | 5.6 | 1.65E−07 | 0.21 | ZNF787 | 3.76 | 7.87E−04 | 0.17 | GTPBP6 | 2.65 | 2.69E−02 | 0.13 |
| NBEAL1 | 11.85 | 7.46E−31 | 0.34 | SGCD | 5.59 | 1.76E−07 | 1.03 | PHB2 | 3.76 | 7.80E−04 | 0.15 | SGCB | 2.65 | 2.69E−02 | 0.11 |
| ATP6V1A | 11.84 | 7.95E−31 | 0.76 | TMEM9B | 5.59 | 1.74E−07 | 0.77 | SRRM1 | 3.76 | 7.79E−04 | 0.11 | RNF169 | 2.65 | 2.67E−02 | 0.06 |
| FN1 | 11.84 | 8.09E−31 | 0.62 | LIPA | 5.59 | 1.81E−07 | 0.59 | OSBPL5 | 3.76 | 7.87E−04 | 0.09 | RBM14-RBM4 | 2.65 | 2.68E−02 | 0.03 |
| PCDH11X | 11.83 | 9.38E−31 | 1.83 | CDKL3 | 5.59 | 1.73E−07 | 0.56 | GTPBP4 | 3.76 | 7.90E−04 | 0.04 | UTP14A | 2.65 | 2.69E−02 | 0.03 |
| KDELR1 | 11.83 | 9.32E−31 | 1.11 | CIRL-AS1 | 5.59 | 1.74E−07 | 0.49 | UNC79 | 3.75 | 8.13E−04 | 1.01 | USP45 | 2.65 | 2.68E−02 | 0 |
| NBEA | 11.82 | 9.87E−31 | 0.62 | NAA20 | 5.59 | 1.77E−07 | 0.44 | ZNF486 | 3.75 | 8.36E−04 | 0.87 | TRABD | 2.65 | 2.70E−02 | 0 |
| COL1A1 | 11.82 | 9.80E−31 | 0.52 | PTPRD | 5.59 | 1.81E−07 | 0.4 | TXNDC17 | 3.75 | 8.13E−04 | 0.62 | NAT10 | 2.65 | 2.68E−02 | −0.02 |
| VAMP8 | 11.8 | 1.34E−30 | 0.98 | ACSF2 | 5.59 | 1.75E−07 | 0.36 | SIPA1 | 3.75 | 8.12E−04 | 0.51 | ISG20L2 | 2.65 | 2.72E−02 | −0.03 |
| KCNK6 | 11.79 | 1.43E−30 | 0.83 | ATP1A1 | 5.59 | 1.81E−07 | 0.28 | CDCA7L | 3.75 | 8.13E−04 | 0.51 | NUP43 | 2.65 | 2.69E−02 | −0.04 |
| ZNF708 | 11.78 | 1.69E−30 | 0.84 | ZNF577 | 5.58 | 1.84E−07 | 0.61 | RNASEH2C | 3.75 | 8.16E−04 | 0.48 | HSD17B10 | 2.64 | 2.75E−02 | 0.68 |
| FAM129A | 11.74 | 2.55E−30 | 1.3 | C5AR1 | 5.58 | 1.83E−07 | 0.32 | STAT5A | 3.75 | 8.35E−04 | 0.34 | WDR5B | 2.64 | 2.78E−02 | 0.64 |
| RPL27 | 11.71 | 3.75E−30 | 1.08 | OPA3 | 5.58 | 1.87E−07 | 0.29 | NFE2L3 | 3.75 | 8.12E−04 | 0.26 | TMEM150A | 2.64 | 2.73E−02 | 0.59 |
| EIF2B5 | 11.7 | 4.26E−30 | 0.95 | ANAPC16 | 5.58 | 1.88E−07 | 0.25 | SNAPC4 | 3.75 | 8.16E−04 | 0.22 | TAC4 | 2.64 | 2.76E−02 | 0.59 |
| ZNRF3 | 11.69 | 4.79E−30 | 0.72 | UNC5B-AS1 | 5.57 | 1.96E−07 | 1.23 | CPSF1 | 3.75 | 8.15E−04 | 0.16 | PHF5A | 2.64 | 2.77E−02 | 0.4 |
| SNHG9 | 11.68 | 5.42E−30 | 1.54 | TMEM132D | 5.57 | 1.96E−07 | 1.09 | POM121C | 3.75 | 8.21E−04 | 0.15 | WDR77 | 2.64 | 2.76E−02 | 0.26 |
| TALD01 | 11.68 | 5.51E−30 | 1.07 | SNRPF | 5.57 | 1.95E−07 | 0.96 | NRP1 | 3.75 | 8.27E−04 | 0.15 | PPP1R11 | 2.64 | 2.72E−02 | 0.21 |
| PFDN5 | 11.67 | 5.82E−30 | 0.8 | SET | 5.57 | 2.02E−07 | 0.29 | PSMF1 | 3.75 | 8.12E−04 | 0.14 | EPHX2 | 2.64 | 2.73E−02 | 0.18 |
| FILIP1L | 11.65 | 7.52E−30 | 0.79 | SMC5-AS1 | 5.57 | 2.01E−07 | 0.28 | CSDE1 | 3.75 | 8.28E−04 | 0.11 | TTC12 | 2.64 | 2.75E−02 | 0.11 |
| NBL1 | 11.59 | 1.58E−29 | 1.14 | RNF114 | 5.56 | 2.15E−07 | 0.25 | HADHB | 3.75 | 8.33E−04 | 0.07 | SKAP1 | 2.64 | 2.74E−02 | 0.08 |
| PSORS1C1 | 11.59 | 1.44E−29 | 0.75 | USP37 | 5.56 | 2.08E−07 | 0.22 | CIAO1 | 3.75 | 8.35E−04 | 0.07 | KLHL42 | 2.64 | 2.74E−02 | 0.04 |
| RCN1 | 11.57 | 1.89E−29 | 1.7 | ATF5 | 5.55 | 2.16E−07 | 1.56 | CHMP2B | 3.75 | 8.22E−04 | 0.06 | GPRC5C | 2.64 | 2.76E−02 | 0 |
| LAMA5 | 11.57 | 1.82E−29 | 0.57 | SLC39A7 | 5.55 | 2.17E−07 | 0.85 | GRM5 | 3.74 | 8.46E−04 | 1.7 | ZNF84 | 2.64 | 2.72E−02 | −0.03 |
| COL1A2 | 11.57 | 1.83E−29 | 0.52 | COL11A1 | 5.55 | 2.26E−07 | 0.76 | CPTP | 3.74 | 8.38E−04 | 1.4 | SLC19A2 | 2.64 | 2.72E−02 | −0.04 |
| NAPRT | 11.55 | 2.40E−29 | 0.76 | DLC1 | 5.55 | 2.20E−07 | 0.42 | TMEM203 | 3.74 | 8.46E−04 | 0.94 | ZNF160 | 2.64 | 2.76E−02 | −0.07 |
| P4HB | 11.53 | 3.12E−29 | 0.68 | VRK3 | 5.55 | 2.15E−07 | 0.33 | FAM71F2 | 3.74 | 8.49E−04 | 0.7 | AC091153.4 | 2.63 | 2.79E−02 | 3.59 |
| NNMT | 11.52 | 3.48E−29 | 1.01 | BEAN1 | 5.55 | 2.20E−07 | 0.3 | EMP3 | 3.74 | 8.40E−04 | 0.64 | LSP1 | 2.63 | 2.82E−02 | 1.83 |
| WDFY1 | 11.52 | 3.43E−29 | 0.6 | EIF4ENIF1 | 5.55 | 2.19E−07 | 0.28 | GHITM | 3.74 | 8.48E−04 | 0.31 | CACNA1B | 2.63 | 2.86E−02 | 1.23 |
| SSR4 | 11.49 | 4.58E−29 | 1.56 | TMEM248 | 5.55 | 2.26E−07 | 0.25 | TCP11L2 | 3.74 | 8.47E−04 | 0.3 | AQP1 | 2.63 | 2.80E−02 | 0.89 |
| GDA | 11.49 | 4.48E−29 | 0.65 | FAM49B | 5.55 | 2.26E−07 | 0.18 | PRR12 | 3.74 | 8.64E−04 | 0.24 | OASL | 2.63 | 2.80E−02 | 0.85 |
| TMCC1 | 11.49 | 4.47E−29 | 0.38 | PRNP | 5.54 | 2.31E−07 | 0.91 | ZNF444 | 3.74 | 8.66E−04 | 0.13 | UCK1 | 2.63 | 2.79E−02 | 0.74 |
| RPL23 | 11.46 | 6.91E−29 | 0.89 | PPIL6 | 5.54 | 2.30E−07 | 0.76 | STK17A | 3.74 | 8.60E−04 | 0.1 | TUSC2 | 2.63 | 2.82E−02 | 0.57 |
| SRPK1 | 11.46 | 6.51E−29 | 0.56 | EFCAB6 | 5.54 | 2.32E−07 | 0.46 | TNFAIP8L3 | 3.73 | 8.85E−04 | 0.66 | NDOR1 | 2.63 | 2.80E−02 | 0.35 |
| CLDN4 | 11.44 | 8.31E−29 | 0.63 | NOC2L | 5.54 | 2.31E−07 | 0.44 | SLC35E3 | 3.73 | 8.91E−04 | 0.21 | CTNS | 2.63 | 2.81E−02 | 0.21 |
| ARL17A | 11.4 | 1.25E−28 | 0.95 | RBM19 | 5.54 | 2.35E−07 | 0.44 | TFG | 3.73 | 8.95E−04 | 0.14 | IRAK2 | 2.63 | 2.79E−02 | 0.17 |
| SEZ6L2 | 11.39 | 1.47E−28 | 1.46 | PEPD | 5.54 | 2.31E−07 | 0.42 | RPS27L | 3.73 | 8.86E−04 | 0.11 | SDR39U1 | 2.63 | 2.83E−02 | 0.15 |
| RPS13 | 11.39 | 1.47E−28 | 1.08 | ZNF264 | 5.54 | 2.29E−07 | 0.4 | EP400 | 3.73 | 8.80E−04 | 0.1 | RAP2C-AS1 | 2.63 | 2.82E−02 | 0.12 |
| DAPK3 | 11.39 | 1.43E−28 | 0.92 | DNPEP | 5.54 | 2.35E−07 | 0.39 | CASC3 | 3.73 | 8.98E−04 | 0.1 | FAF2 | 2.63 | 2.86E−02 | 0.08 |
| FKBP2 | 11.37 | 1.75E−28 | 1.17 | GARNL3 | 5.54 | 2.39E−07 | 0.36 | CETN2 | 3.72 | 9.13E−04 | 1.22 | CLHC1 | 2.63 | 2.81E−02 | 0.01 |
| LING01 | 11.36 | 1.95E−28 | 0.59 | ARHGEF10L | 5.54 | 2.29E−07 | 0.28 | TMEM92 | 3.72 | 9.32E−04 | 0.87 | ZNHIT6 | 2.63 | 2.79E−02 | −0.02 |
| CHN2 | 11.34 | 2.66E−28 | 2.33 | MFSD14B | 5.54 | 2.37E−07 | 0.27 | RAB34 | 3.72 | 9.06E−04 | 0.7 | NLRC5 | 2.63 | 2.80E−02 | −0.04 |
| EPB42 | 11.34 | 2.45E−28 | 1.79 | SEC16A | 5.54 | 2.32E−07 | 0.2 | FAM32A | 3.72 | 9.32E−04 | 0.49 | SMARCB1 | 2.63 | 2.79E−02 | −0.06 |
| LRRIQ1 | 11.33 | 2.81E−28 | 2.18 | SYNM | 5.53 | 2.52E−07 | 2.74 | IRF1 | 3.72 | 9.20E−04 | 0.31 | AC100830.3 | 2.62 | 2.91E−02 | 2.81 |
| P3H4 | 11.33 | 2.91E−28 | 1.55 | ZNF350-AS1 | 5.53 | 2.50E−07 | 1.83 | NAAA | 3.72 | 9.05E−04 | 0.26 | FAXC | 2.62 | 2.92E−02 | 1.15 |
| CD74 | 11.32 | 3.17E−28 | 1.39 | CCDC180 | 5.53 | 2.48E−07 | 1.02 | ZCCHC2 | 3.72 | 9.22E−04 | 0.22 | MSH4 | 2.62 | 2.92E−02 | 1.07 |
| LRSAM1 | 11.32 | 3.38E−28 | 0.65 | C19orf24 | 5.53 | 2.41E−07 | 0.93 | SMARCD2 | 3.72 | 9.34E−04 | 0.22 | TMEM109 | 2.62 | 2.88E−02 | 0.85 |
| RPLP0 | 11.29 | 4.33E−28 | 0.64 | IDH3G | 5.53 | 2.48E−07 | 0.84 | TOB1 | 3.72 | 9.28E−04 | 0.18 | MAP1B | 2.62 | 2.87E−02 | 0.27 |
| MRPS34 | 11.27 | 5.40E−28 | 1.33 | FYTTD1 | 5.53 | 2.47E−07 | 0.38 | FAM174B | 3.72 | 9.06E−04 | 0.15 | INAFM1 | 2.62 | 2.93E−02 | 0.2 |
| RPL11 | 11.24 | 7.58E−28 | 0.76 | SURF4 | 5.53 | 2.41E−07 | 0.34 | RFX3-AS1 | 3.72 | 9.06E−04 | 0.13 | SNX6 | 2.62 | 2.88E−02 | 0.11 |
| DTX3L | 11.23 | 9.14E−28 | 1.04 | STARD3 | 5.53 | 2.52E−07 | 0.31 | FAM76A | 3.72 | 9.15E−04 | 0.13 | PTPN22 | 2.62 | 2.92E−02 | 0.11 |
| RPS9 | 11.23 | 8.55E−28 | 0.61 | LOXL1-AS1 | 5.52 | 2.55E−07 | 1.36 | UBXN7 | 3.72 | 9.03E−04 | 0.11 | RTN3 | 2.62 | 2.91E−02 | 0.1 |
| TMED3 | 11.21 | 1.04E−27 | 0.92 | SCNM1 | 5.52 | 2.60E−07 | 0.65 | CATSPERD | 3.71 | 9.49E−04 | 1.83 | CDC23 | 2.62 | 2.87E−02 | 0.05 |
| TBC1D2 | 11.21 | 1.11E−27 | 0.68 | EVI5L | 5.52 | 2.54E−07 | 0.47 | TMEM254-AS1 | 3.71 | 9.64E−04 | 1.62 | MCF2L | 2.62 | 2.91E−02 | 0.03 |
| RHOD | 11.18 | 1.57E−27 | 1.15 | KIF9-AS1 | 5.52 | 2.55E−07 | 0.29 | FAHD1 | 3.71 | 9.55E−04 | 0.56 | TUBA1C | 2.62 | 2.91E−02 | −0.01 |
| DEPDC5 | 11.17 | 1.66E−27 | 2.98 | IRS1 | 5.52 | 2.62E−07 | 0.29 | RNF167 | 3.71 | 9.50E−04 | 0.54 | PSMG4 | 2.62 | 2.88E−02 | −0.06 |
| TCF7L1 | 11.16 | 1.97E−27 | 1.49 | VWA3A | 5.51 | 2.69E−07 | 1.71 | KCNIP4 | 3.71 | 9.46E−04 | 0.36 | INO80E | 2.62 | 2.90E−02 | −0.06 |
| PEBP1 | 11.13 | 2.55E−27 | 1.23 | KIAA2013 | 5.51 | 2.75E−07 | 0.94 | CRACR2A | 3.71 | 9.39E−04 | 0.3 | C16orf96 | 2.61 | 2.96E−02 | 2.06 |
| PLXNA1 | 11.13 | 2.70E−27 | 0.96 | SEMA5A | 5.51 | 2.71E−07 | 0.8 | KLHDC3 | 3.71 | 9.46E−04 | 0.3 | LINC01494 | 2.61 | 2.98E−02 | 1.89 |
| C4orf36 | 11.12 | 2.79E−27 | 2.53 | GGACT | 5.51 | 2.78E−07 | 0.69 | HABP4 | 3.71 | 9.41E−04 | 0.23 | PODNL1 | 2.61 | 2.99E−02 | 1.59 |
| GABPB1-AS1 | 11.12 | 3.06E−27 | 0.68 | DNASE1L1 | 5.51 | 2.75E−07 | 0.66 | SLC37A1 | 3.71 | 9.46E−04 | 0.18 | APOBEC3G | 2.61 | 3.01E−02 | 1.21 |
| TSC22D3 | 11.12 | 3.08E−27 | 0.51 | ANTXR1 | 5.51 | 2.72E−07 | 0.63 | HPS1 | 3.71 | 9.59E−04 | 0.16 | DNAJC12 | 2.61 | 3.02E−02 | 0.93 |
| CLUAP1 | 11.1 | 3.71E−27 | 1.08 | MAPK3 | 5.51 | 2.79E−07 | 0.62 | AC002066.1 | 3.71 | 9.43E−04 | 0.15 | RGS5 | 2.61 | 2.99E−02 | 0.65 |
| CD63 | 11.07 | 5.17E−27 | 0.78 | SAMD9L | 5.51 | 2.70E−07 | 0.37 | ZC3H7B | 3.71 | 9.37E−04 | 0.13 | EMILIN1 | 2.61 | 2.99E−02 | 0.55 |
| RPL32 | 11.06 | 5.51E−27 | 0.83 | DUSP18 | 5.51 | 2.72E−07 | 0.3 | SMG9 | 3.71 | 9.38E−04 | 0.1 | TSR2 | 2.61 | 3.01E−02 | 0.52 |
| RPS15A | 11.03 | 7.67E−27 | 1.38 | COQ4 | 5.51 | 2.69E−07 | 0.25 | VEZF1 | 3.71 | 9.39E−04 | 0.1 | APOL3 | 2.61 | 2.99E−02 | 0.42 |
| SLC9C1 | 11.02 | 9.21E−27 | 0.84 | REL | 5.51 | 2.68E−07 | 0.22 | MAP3K19 | 3.7 | 9.86E−04 | 1.09 | DBI | 2.61 | 3.01E−02 | 0.35 |
| ABCA7 | 11.02 | 8.81E−27 | 0.65 | VAMP5 | 5.5 | 2.88E−07 | 0.91 | C19orf18 | 3.7 | 9.77E−04 | 0.88 | RNF144B | 2.61 | 2.99E−02 | 0.19 |
| SSH1 | 11.01 | 9.65E−27 | 0.77 | MMAB | 5.5 | 2.92E−07 | 0.71 | SLC6A16 | 3.7 | 9.93E−04 | 0.7 | RNF6 | 2.61 | 2.99E−02 | 0.19 |
| RPL23A | 11 | 1.12E−26 | 0.96 | GNPTAB | 5.5 | 2.86E−07 | 0.33 | ISYNA1 | 3.7 | 9.86E−04 | 0.45 | RBMX | 2.61 | 2.96E−02 | 0.11 |
| MRPL41 | 10.98 | 1.41E−26 | 1.12 | ABI3BP | 5.5 | 2.90E−07 | 0.24 | NUDT16 | 3.7 | 1.00E−03 | 0.44 | BAALC-AS1 | 2.61 | 2.99E−02 | 0.11 |
| MEG3 | 10.97 | 1.46E−26 | 3.25 | FLRT3 | 5.49 | 3.05E−07 | 1.06 | PPIE | 3.7 | 9.81E−04 | 0.36 | AHI1 | 2.61 | 2.98E−02 | 0.09 |
| RPL24 | 10.97 | 1.57E−26 | 0.95 | ASRGL1 | 5.49 | 3.02E−07 | 0.68 | CLDN15 | 3.7 | 9.81E−04 | 0.34 | B3GNT2 | 2.61 | 2.96E−02 | 0.07 |
| CLN8 | 10.94 | 2.10E−26 | 3 | FSCN1 | 5.49 | 3.05E−07 | 0.62 | CHID1 | 3.7 | 9.73E−04 | 0.3 | CSMD1 | 2.61 | 2.95E−02 | 0.06 |
| PHLDB2 | 10.94 | 2.12E−26 | 0.82 | MTG2 | 5.49 | 3.03E−07 | 0.32 | GPR107 | 3.7 | 9.79E−04 | 0.13 | MAML1 | 2.61 | 2.96E−02 | 0.06 |
| ZER1 | 10.94 | 2.03E−26 | 0.64 | CCDC88C | 5.48 | 3.24E−07 | 0.29 | MARCKS | 3.7 | 9.80E−04 | 0.12 | SGPL1 | 2.61 | 2.97E−02 | 0.06 |
| DTD1 | 10.92 | 2.48E−26 | 2.18 | RBBP8 | 5.48 | 3.23E−07 | 0.28 | RPA1 | 3.7 | 9.79E−04 | 0.1 | NPC1 | 2.61 | 2.95E−02 | 0.02 |
| UQCRQ | 10.92 | 2.59E−26 | 1.36 | EIF3B | 5.48 | 3.18E−07 | 0.25 | MKNK1 | 3.7 | 9.99E−04 | 0.09 | CDK11A | 2.61 | 2.99E−02 | 0 |
| YARS | 10.89 | 3.40E−26 | 0.74 | NPC2 | 5.47 | 3.40E−07 | 0.68 | SMIM3 | 3.69 | 1.03E−03 | 1.09 | ESRRA | 2.61 | 2.99E−02 | −0.01 |
| ZNF320 | 10.87 | 4.48E−26 | 3.39 | SCML1 | 5.47 | 3.31E−07 | 0.3 | ZNF358 | 3.69 | 1.04E−03 | 1.03 | PHKG2 | 2.61 | 2.99E−02 | −0.02 |
| COX6B1 | 10.86 | 5.04E−26 | 1.27 | PIWIL4 | 5.47 | 3.44E−07 | 0.26 | TMEM223 | 3.69 | 1.03E−03 | 0.56 | BEND6 | 2.6 | 3.09E−02 | 2.11 |
| SEC61B | 10.86 | 4.90E−26 | 0.93 | ST6GALNAC2 | 5.46 | 3.51E−07 | 0.79 | CABP4 | 3.69 | 1.02E−03 | 0.5 | EFHD1 | 2.6 | 3.04E−02 | 1.43 |
| SYNE1 | 10.86 | 4.90E−26 | 0.86 | SCAF1 | 5.46 | 3.68E−07 | 0.7 | EFCAB5 | 3.69 | 1.02E−03 | 0.46 | CORO2B | 2.6 | 3.07E−02 | 1.22 |
| CATSPER2 | 10.86 | 5.04E−26 | 0.62 | PCBP1 | 5.46 | 3.56E−07 | 0.4 | CHSY3 | 3.69 | 1.01E−03 | 0.28 | TRIAP1 | 2.6 | 3.02E−02 | 1 |
| PITPNC1 | 10.86 | 5.01E−26 | 0.43 | KXD1 | 5.46 | 3.52E−07 | 0.38 | C3orf38 | 3.69 | 1.03E−03 | 0.19 | CITED2 | 2.6 | 3.10E−02 | 0.69 |
| ARL6IP4 | 10.85 | 5.40E−26 | 0.75 | ANXA4 | 5.46 | 3.64E−07 | 0.18 | GLUD1 | 3.69 | 1.04E−03 | 0.18 | IL3RA | 2.6 | 3.06E−02 | 0.33 |
| NPRL3 | 10.84 | 5.83E−26 | 0.63 | LIMD2 | 5.45 | 3.74E−07 | 3.83 | SENP5 | 3.69 | 1.02E−03 | 0.17 | AAED1 | 2.6 | 3.06E−02 | 0.28 |
| CTGF | 10.83 | 7.02E−26 | 0.8 | DZIP1 | 5.45 | 3.74E−07 | 1.54 | TMEM8A | 3.69 | 1.01E−03 | 0.16 | MATR3 | 2.6 | 3.04E−02 | 0.26 |
| KRT17 | 10.82 | 7.30E−26 | 0.68 | AGPAT1 | 5.45 | 3.74E−07 | 1.05 | NBR1 | 3.69 | 1.02E−03 | 0.15 | WDR81 | 2.6 | 3.06E−02 | 0.25 |
| IQCK | 10.82 | 7.39E−26 | 0.6 | FBXO44 | 5.45 | 3.88E−07 | 0.99 | REEP5 | 3.69 | 1.01E−03 | 0.12 | KLHL13 | 2.6 | 3.03E−02 | 0.23 |
| FAM83A | 10.8 | 9.29E−26 | 0.85 | LAMTOR2 | 5.45 | 3.70E−07 | 0.84 | METTL16 | 3.69 | 1.03E−03 | 0.12 | TAF6L | 2.6 | 3.05E−02 | 0.17 |
| B2M | 10.76 | 1.48E−25 | 0.53 | MAZ | 5.45 | 3.71E−07 | 0.59 | LAMP2 | 3.69 | 1.03E−03 | 0.11 | KRT23 | 2.6 | 3.05E−02 | 0.16 |
| OST4 | 10.74 | 1.79E−25 | 1.6 | SNHG7 | 5.45 | 3.83E−07 | 0.43 | CTTNBP2NL | 3.69 | 1.02E−03 | 0.09 | HACD1 | 2.6 | 3.07E−02 | 0.14 |
| PDIA3 | 10.74 | 1.83E−25 | 0.71 | TRIM22 | 5.45 | 3.88E−07 | 0.42 | SDC3 | 3.68 | 1.08E−03 | 0.82 | NPEPL1 | 2.6 | 3.05E−02 | 0.12 |
| MMP23B | 10.73 | 1.98E−25 | 1.29 | PARK7 | 5.45 | 3.86E−07 | 0.38 | CWC15 | 3.68 | 1.07E−03 | 0.4 | CCT7 | 2.6 | 3.03E−02 | 0.1 |
| STK38 | 10.73 | 2.06E−25 | 0.48 | TGFBI | 5.44 | 4.04E−07 | 0.84 | ANKFN1 | 3.68 | 1.06E−03 | 0.23 | CD2AP | 2.6 | 3.08E−02 | 0.09 |
| CAPNS1 | 10.72 | 2.24E−25 | 0.97 | CELSR1 | 5.44 | 4.09E−07 | 0.26 | FMR1 | 3.68 | 1.06E−03 | 0.21 | 2-Mar | 2.6 | 3.07E−02 | 0.05 |
| AEBP1 | 10.71 | 2.44E−25 | 1.16 | CHST11 | 5.44 | 4.02E−07 | 0.25 | HDAC5 | 3.68 | 1.07E−03 | 0.16 | DNAJC9 | 2.6 | 3.07E−02 | 0.04 |
| SLC27A4 | 10.71 | 2.39E−25 | 0.98 | CDK10 | 5.44 | 3.89E−07 | 0.21 | FAM157C | 3.68 | 1.07E−03 | 0.09 | ALG8 | 2.6 | 3.09E−02 | 0.01 |
| CDH4 | 10.7 | 2.68E−25 | 2.1 | UQCRFS1 | 5.43 | 4.32E−07 | 0.87 | ZNF771 | 3.67 | 1.11E−03 | 1.12 | CHIC1 | 2.6 | 3.03E−02 | 0 |
| COL6A2 | 10.7 | 2.87E−25 | 1.05 | LINC01524 | 5.43 | 4.13E−07 | 0.71 | ADAL | 3.67 | 1.10E−03 | 0.58 | MOV10 | 2.6 | 3.07E−02 | 0 |
| ZNF429 | 10.69 | 3.14E−25 | 0.95 | LRFN4 | 5.43 | 4.19E−07 | 0.68 | ZNF778 | 3.67 | 1.12E−03 | 0.53 | MGAT4B | 2.6 | 3.05E−02 | −0.09 |
| CEACAM6 | 10.69 | 3.05E−25 | 0.82 | VPS4A | 5.43 | 4.23E−07 | 0.46 | KIF9 | 3.67 | 1.12E−03 | 0.4 | SUPT7L | 2.6 | 3.10E−02 | −0.1 |
| TMEM120B | 10.68 | 3.32E−25 | 0.78 | DYSF | 5.42 | 4.52E−07 | 1.43 | ZP3 | 3.67 | 1.09E−03 | 0.37 | ALDOB | 2.59 | 3.13E−02 | 1.44 |
| ROBO2 | 10.65 | 4.88E−25 | 1.18 | TSR3 | 5.42 | 4.44E−07 | 1.06 | H2AFZ | 3.67 | 1.10E−03 | 0.34 | IQCD | 2.59 | 3.15E−02 | 1.02 |
| PLCE1 | 10.65 | 4.63E−25 | 0.78 | ZNHIT1 | 5.42 | 4.56E−07 | 0.47 | PDCD7 | 3.67 | 1.11E−03 | 0.28 | TIGD5 | 2.59 | 3.14E−02 | 0.68 |
| ITPKC | 10.65 | 4.66E−25 | 0.62 | C2orf68 | 5.42 | 4.56E−07 | 0.43 | MPV17 | 3.67 | 1.10E−03 | 0.2 | CCDC62 | 2.59 | 3.10E−02 | 0.58 |
| TNS3 | 10.65 | 4.54E−25 | 0.46 | CCT4 | 5.42 | 4.35E−07 | 0.23 | RNMT | 3.67 | 1.09E−03 | 0.14 | PHYHD1 | 2.59 | 3.14E−02 | 0.41 |
| CMIP | 10.64 | 5.04E−25 | 0.39 | ALDH18A1 | 5.42 | 4.46E−07 | 0.21 | WHAMM | 3.67 | 1.10E−03 | 0.07 | KATNAL2 | 2.59 | 3.14E−02 | 0.32 |
| PAK4 | 10.62 | 6.27E−25 | 0.69 | ATP7A | 5.41 | 4.69E−07 | 2.44 | TGFB111 | 3.66 | 1.14E−03 | 0.8 | IGFBP3 | 2.59 | 3.13E−02 | 0.18 |
| LTBR | 10.61 | 6.96E−25 | 0.58 | ZNF880 | 5.41 | 4.79E−07 | 1.85 | PRR15L | 3.66 | 1.14E−03 | 0.36 | TGFA | 2.59 | 3.17E−02 | 0.05 |
| COX7C | 10.6 | 8.11E−25 | 1.18 | EFHC2 | 5.41 | 4.59E−07 | 0.98 | ADI1 | 3.66 | 1.15E−03 | 0.26 | NDUFA5 | 2.59 | 3.10E−02 | 0 |
| SH3PXD2A | 10.6 | 7.80E−25 | 0.49 | FEM1B | 5.41 | 4.78E−07 | 0.46 | IL6R | 3.66 | 1.12E−03 | 0.17 | SFT2D2 | 2.59 | 3.13E−02 | 0 |
| TAF8 | 10.59 | 8.65E−25 | 1 | RPS21 | 5.41 | 4.64E−07 | 0.43 | MTF1 | 3.66 | 1.14E−03 | 0.14 | OSER1 | 2.59 | 3.14E−02 | 0 |
| THBS1 | 10.58 | 1.01E−24 | 1.14 | PEX6 | 5.41 | 4.68E−07 | 0.42 | ZNF552 | 3.66 | 1.16E−03 | 0.13 | UPF3B | 2.59 | 3.16E−02 | −0.01 |
| CNTNAP2 | 10.58 | 9.41E−25 | 1.01 | AAMP | 5.41 | 4.59E−07 | 0.34 | RAB11FIP4 | 3.66 | 1.16E−03 | 0.09 | H2AFV | 2.59 | 3.16E−02 | −0.01 |
| RPS4X | 10.58 | 9.25E−25 | 0.82 | LINC00894 | 5.41 | 4.80E−07 | 0.33 | TRIM38 | 3.66 | 1.14E−03 | 0.06 | ZFP57 | 2.58 | 3.24E−02 | 3.27 |
| SH3BGRL3 | 10.57 | 1.06E−24 | 1.04 | ARPC5 | 5.41 | 4.59E−07 | 0.31 | NAT14 | 3.65 | 1.20E−03 | 0.96 | DNAH8 | 2.58 | 3.23E−02 | 1.3 |
| HSPA5 | 10.56 | 1.25E−24 | 0.9 | CLASRP | 5.41 | 4.77E−07 | 0.31 | LIPC | 3.65 | 1.18E−03 | 0.93 | SNRNP25 | 2.58 | 3.19E−02 | 1.1 |
| RILPL1 | 10.55 | 1.30E−24 | 0.87 | CHMP4B | 5.41 | 4.57E−07 | 0.3 | LINC01252 | 3.65 | 1.20E−03 | 0.66 | ANAPC15 | 2.58 | 3.22E−02 | 0.89 |
| MCFD2 | 10.55 | 1.39E−24 | 0.83 | PPARA | 5.41 | 4.64E−07 | 0.23 | PLB1 | 3.65 | 1.17E−03 | 0.52 | GPR34 | 2.58 | 3.21E−02 | 0.78 |
| EVA1C | 10.55 | 1.35E−24 | 0.48 | ITM2C | 5.4 | 4.94E−07 | 1.23 | HEG1 | 3.65 | 1.17E−03 | 0.46 | EIF3C | 2.58 | 3.22E−02 | 0.57 |
| SDC1 | 10.54 | 1.51E−24 | 0.8 | MAMDC2-AS1 | 5.4 | 5.06E−07 | 0.47 | NEU1 | 3.65 | 1.17E−03 | 0.4 | CASC8 | 2.58 | 3.24E−02 | 0.55 |
| MARK4 | 10.54 | 1.52E−24 | 0.66 | TLE3 | 5.4 | 4.87E−07 | 0.32 | CDON | 3.65 | 1.18E−03 | 0.28 | ADGRG7 | 2.58 | 3.23E−02 | 0.5 |
| DLG2 | 10.53 | 1.65E−24 | 0.79 | SLC47A1 | 5.39 | 5.31E−07 | 1.18 | ABTB1 | 3.65 | 1.19E−03 | 0.22 | NUP21OL | 2.58 | 3.19E−02 | 0.44 |
| RPL30 | 10.53 | 1.59E−24 | 0.73 | NEK8 | 5.39 | 5.29E−07 | 0.67 | POLM | 3.65 | 1.19E−03 | 0.2 | MOSPD1 | 2.58 | 3.18E−02 | 0.42 |
| LGALS1 | 10.52 | 1.90E−24 | 1.38 | ZNF16 | 5.39 | 5.10E−07 | 0.62 | METAP1D | 3.65 | 1.19E−03 | 0.19 | MRPL16 | 2.58 | 3.21E−02 | 0.38 |
| ATG12 | 10.52 | 1.74E−24 | 0.58 | THNSL2 | 5.39 | 5.10E−07 | 0.62 | NELFB | 3.65 | 1.18E−03 | 0.17 | NEK5 | 2.58 | 3.22E−02 | 0.32 |
| USP49 | 10.5 | 2.27E−24 | 0.81 | BETIL | 5.39 | 5.28E−07 | 0.49 | C20orf194 | 3.65 | 1.18E−03 | 0.17 | ASB13 | 2.58 | 3.18E−02 | 0.22 |
| HIP1R | 10.5 | 2.27E−24 | 0.66 | SPTLC3 | 5.39 | 5.11E−07 | 0.42 | MTA1 | 3.65 | 1.20E−03 | 0.17 | NDUFS3 | 2.58 | 3.18E−02 | 0.16 |
| KIFC3 | 10.5 | 2.16E−24 | 0.61 | LSR | 5.39 | 5.15E−07 | 0.29 | SFXN3 | 3.65 | 1.19E−03 | 0.1 | ZNF354A | 2.58 | 3.21E−02 | 0.15 |
| PAX8 | 10.49 | 2.34E−24 | 0.73 | CREB3L2 | 5.39 | 5.31E−07 | 0.28 | PSMD12 | 3.65 | 1.17E−03 | 0.06 | DZANK1 | 2.58 | 3.25E−02 | 0.08 |
| MUC13 | 10.48 | 2.67E−24 | 1.3 | TMED10 | 5.39 | 5.09E−07 | 0.25 | SLC2A12 | 3.64 | 1.21E−03 | 1.21 | WBP1L | 2.58 | 3.23E−02 | 0.02 |
| PRKCI | 10.48 | 2.77E−24 | 0.46 | MBD4 | 5.39 | 5.31E−07 | 0.22 | SPHK2 | 3.64 | 1.24E−03 | 0.52 | MTERF4 | 2.58 | 3.18E−02 | 0 |
| PDLIM7 | 10.48 | 2.79E−24 | 0.45 | UQCC3 | 5.38 | 5.38E−07 | 0.87 | MRPL18 | 3.64 | 1.24E−03 | 0.51 | CHD3 | 2.58 | 3.25E−02 | −0.01 |
| GPX4 | 10.46 | 3.54E−24 | 0.6 | TMEM160 | 5.38 | 5.54E−07 | 0.84 | SLC41A3 | 3.64 | 1.24E−03 | 0.48 | CCDC120 | 2.58 | 3.21E−02 | −0.1 |
| ZNF565 | 10.45 | 3.73E−24 | 0.59 | OGFOD1 | 5.38 | 5.53E−07 | 0.39 | RSAD1 | 3.64 | 1.22E−03 | 0.35 | ANKRD1 | 2.57 | 3.29E−02 | 1.33 |
| CKAP4 | 10.43 | 4.40E−24 | 1.55 | FAM81A | 5.38 | 5.51E−07 | 0.38 | LINC01088 | 3.64 | 1.23E−03 | 0.32 | HOXB-AS1 | 2.57 | 3.33E−02 | 1.08 |
| CEACAM19 | 10.43 | 4.64E−24 | 1.08 | ZFP36 | 5.38 | 5.51E−07 | 0.37 | VNN1 | 3.64 | 1.24E−03 | 0.15 | SAXO2 | 2.57 | 3.29E−02 | 0.93 |
| RHOQ | 10.43 | 4.49E−24 | 0.93 | SPARC | 5.38 | 5.47E−07 | 0.29 | APLF | 3.64 | 1.25E−03 | 0.15 | FEM1A | 2.57 | 3.28E−02 | 0.79 |
| UBE2I | 10.41 | 5.87E−24 | 0.51 | UNC93B1 | 5.38 | 5.57E−07 | 0.28 | PEX13 | 3.64 | 1.21E−03 | 0.1 | MAPK11 | 2.57 | 3.32E−02 | 0.6 |
| TRIM5 | 10.4 | 6.40E−24 | 1.32 | CORO2A | 5.38 | 5.44E−07 | 0.26 | BOLA3 | 3.63 | 1.28E−03 | 1.46 | HINT2 | 2.57 | 3.32E−02 | 0.45 |
| CYTH3 | 10.4 | 6.54E−24 | 0.71 | SSNA1 | 5.37 | 5.95E−07 | 0.75 | KIAA0319 | 3.63 | 1.26E−03 | 1.01 | WDR74 | 2.57 | 3.29E−02 | 0.19 |
| DNAJB11 | 10.39 | 6.57E−24 | 1.57 | BID | 5.37 | 5.83E−07 | 0.44 | TMEM139 | 3.63 | 1.27E−03 | 0.93 | SPEF2 | 2.57 | 3.26E−02 | 0.12 |
| ZFP36L1 | 10.38 | 8.02E−24 | 0.42 | BCL2L13 | 5.37 | 5.86E−07 | 0.22 | C15orf48 | 3.63 | 1.27E−03 | 0.51 | TMOD3 | 2.57 | 3.27E−02 | 0.12 |
| NUDT3 | 10.37 | 8.22E−24 | 1.23 | GPM6A | 5.36 | 5.99E−07 | 1.45 | IFT20 | 3.63 | 1.30E−03 | 0.19 | MORN1 | 2.57 | 3.31E−02 | 0.12 |
| IRF8 | 10.36 | 9.12E−24 | 1.98 | RASGRP3 | 5.36 | 6.08E−07 | 0.72 | STIM1 | 3.63 | 1.27E−03 | 0.16 | TM7SF3 | 2.57 | 3.26E−02 | 0.11 |
| FTL | 10.35 | 1.09E−23 | 0.61 | ATP6AP2 | 5.36 | 6.19E−07 | 0.62 | TMC5 | 3.63 | 1.30E−03 | 0.16 | KIF1B | 2.57 | 3.33E−02 | 0.1 |
| MMP14 | 10.34 | 1.14E−23 | 0.85 | CREB5 | 5.36 | 6.16E−07 | 0.37 | SCARB2 | 3.63 | 1.28E−03 | 0.15 | MALSU1 | 2.57 | 3.28E−02 | 0.05 |
| SLC16A1-AS1 | 10.33 | 1.25E−23 | 0.98 | TSG101 | 5.36 | 6.00E−07 | 0.24 | ZNF26 | 3.63 | 1.28E−03 | 0.07 | IDH3A | 2.57 | 3.29E−02 | 0.05 |
| TP63 | 10.33 | 1.22E−23 | 0.9 | CEP126 | 5.36 | 6.11E−07 | 0.22 | LHB | 3.62 | 1.34E−03 | 1.58 | C5orf24 | 2.57 | 3.32E−02 | 0.05 |
| NABP1 | 10.33 | 1.31E−23 | 0.75 | GALNTL6 | 5.35 | 6.29E−07 | 0.99 | PDZD7 | 3.62 | 1.33E−03 | 1.07 | APEX1 | 2.57 | 3.27E−02 | 0.04 |
| KRT7 | 10.3 | 1.80E−23 | 0.42 | TSPYL1 | 5.35 | 6.46E−07 | 0.97 | CERCAM | 3.62 | 1.30E−03 | 0.96 | SRP68 | 2.57 | 3.28E−02 | 0.01 |
| SYNP02 | 10.29 | 1.89E−23 | 1.55 | ZNF347 | 5.35 | 6.50E−07 | 0.96 | CHODL | 3.62 | 1.30E−03 | 0.86 | GNE | 2.57 | 3.29E−02 | −0.01 |
| CASC9 | 10.29 | 1.95E−23 | 0.78 | MR1 | 5.35 | 6.55E−07 | 0.7 | NDUFB3 | 3.62 | 1.34E−03 | 0.51 | ZNF718 | 2.57 | 3.32E−02 | −0.01 |
| NBPF14 | 10.28 | 2.23E−23 | 0.75 | VARS | 5.35 | 6.51E−07 | 0.6 | PCYOX1 | 3.62 | 1.30E−03 | 0.47 | ZNF525 | 2.57 | 3.27E−02 | −0.02 |
| ZNF611 | 10.27 | 2.45E−23 | 0.63 | EHD2 | 5.35 | 6.30E−07 | 0.52 | TOMM34 | 3.62 | 1.32E−03 | 0.36 | PRTFDC1 | 2.57 | 3.30E−02 | −0.07 |
| RASSF5 | 10.26 | 2.53E−23 | 1.03 | SARM1 | 5.35 | 6.43E−07 | 0.48 | MALT1 | 3.62 | 1.32E−03 | 0.18 | GRM1 | 2.56 | 3.41E−02 | 2.35 |
| RPL7A | 10.26 | 2.53E−23 | 0.71 | JRK | 5.35 | 6.30E−07 | 0.35 | MLLT3 | 3.62 | 1.30E−03 | 0.16 | SLC22A18AS | 2.56 | 3.37E−02 | 0.99 |
| ELMOD3 | 10.25 | 2.88E−23 | 0.62 | ASCC2 | 5.35 | 6.59E−07 | 0.24 | NPLOC4 | 3.62 | 1.32E−03 | 0.16 | FADS1 | 2.56 | 3.39E−02 | 0.61 |
| TFDP2 | 10.25 | 2.77E−23 | 0.46 | RNF11 | 5.35 | 6.33E−07 | 0.22 | MPP7 | 3.62 | 1.32E−03 | 0.14 | TRABD2B | 2.56 | 3.36E−02 | 0.59 |
| EML4 | 10.25 | 2.87E−23 | 0.44 | ARL13B | 5.35 | 6.51E−07 | 0.21 | PRMT9 | 3.62 | 1.34E−03 | 0.12 | MYPOP | 2.56 | 3.38E−02 | 0.29 |
| VARS2 | 10.24 | 3.12E−23 | 1.44 | DLGAP1-AS1 | 5.34 | 6.68E−07 | 0.8 | KIAA1958 | 3.62 | 1.31E−03 | 0.11 | TIPIN | 2.56 | 3.39E−02 | 0.25 |
| MT-ND4 | 10.23 | 3.70E−23 | 0.7 | GRIK1 | 5.34 | 6.76E−07 | 0.77 | DNAL1 | 3.62 | 1.32E−03 | 0.11 | TCTA | 2.56 | 3.35E−02 | 0.22 |
| LINC01133 | 10.23 | 3.42E−23 | 0.57 | CEBPG | 5.34 | 6.82E−07 | 0.47 | SYTL1 | 3.62 | 1.33E−03 | 0.09 | ASPHD2 | 2.56 | 3.40E−02 | 0.17 |
| SRRM2 | 10.21 | 4.22E−23 | 0.43 | MPHOSPH10 | 5.34 | 6.72E−07 | 0.39 | TLL2 | 3.61 | 1.36E−03 | 1.51 | ATG4D | 2.56 | 3.39E−02 | 0.16 |
| ASTN2 | 10.2 | 4.75E−23 | 0.9 | GBP3 | 5.34 | 6.83E−07 | 0.33 | PPM1E | 3.61 | 1.35E−03 | 1 | ZDHHC24 | 2.56 | 3.39E−02 | 0.13 |
| ZNF76 | 10.18 | 5.78E−23 | 0.9 | LAPTM4B | 5.34 | 6.85E−07 | 0.23 | HMGN5 | 3.61 | 1.37E−03 | 0.58 | TCFL5 | 2.56 | 3.36E−02 | 0.06 |
| NMT1 | 10.18 | 5.98E−23 | 0.51 | FUCA1 | 5.33 | 7.31E−07 | 1.08 | FBL | 3.61 | 1.37E−03 | 0.42 | MYT1L | 2.55 | 3.44E−02 | 1.34 |
| PLEC | 10.18 | 5.98E−23 | 0.36 | CEBPB | 5.33 | 7.05E−07 | 0.32 | TTPAL | 3.61 | 1.39E−03 | 0.33 | CPXM2 | 2.55 | 3.47E−02 | 1.21 |
| RPL21 | 10.17 | 6.56E−23 | 0.79 | LMAN1 | 5.33 | 7.32E−07 | 0.32 | FAM227B | 3.61 | 1.39E−03 | 0.21 | BCO1 | 2.55 | 3.48E−02 | 0.71 |
| RPS24 | 10.17 | 6.56E−23 | 0.53 | ZMPSTE24 | 5.33 | 7.26E−07 | 0.31 | AC011997.1 | 3.61 | 1.35E−03 | 0.16 | ACD | 2.55 | 3.51E−02 | 0.68 |
| MICA | 10.15 | 7.93E−23 | 0.85 | ITPR3 | 5.33 | 7.11E−07 | 0.22 | SH3BP1 | 3.6 | 1.43E−03 | 3.94 | TSPAN6 | 2.55 | 3.43E−02 | 0.52 |
| NR2F2-AS1 | 10.15 | 7.89E−23 | 0.7 | CAP1 | 5.33 | 7.14E−07 | 0.2 | ANKRD54 | 3.6 | 1.44E−03 | 0.52 | PLEKHG7 | 2.55 | 3.49E−02 | 0.3 |
| CACNB2 | 10.14 | 8.56E−23 | 1.03 | TOR4A | 5.32 | 7.60E−07 | 0.85 | TM4SF4 | 3.6 | 1.42E−03 | 0.49 | POU6F1 | 2.55 | 3.51E−02 | 0.2 |
| ACTG2 | 10.13 | 9.89E−23 | 3.64 | RNF187 | 5.32 | 7.48E−07 | 0.73 | FAM196A | 3.6 | 1.40E−03 | 0.17 | ZNF808 | 2.55 | 3.43E−02 | 0.14 |
| RPL3 | 10.13 | 1.00E−22 | 0.67 | PHB | 5.32 | 7.61E−07 | 0.57 | MICALL2 | 3.6 | 1.42E−03 | 0.16 | DENND1A | 2.55 | 3.52E−02 | 0.09 |
| ALDH1A1 | 10.12 | 1.05E−22 | 1.17 | DOK4 | 5.32 | 7.71E−07 | 0.46 | EIF3J | 3.6 | 1.44E−03 | 0.09 | GTF2H2C | 2.55 | 3.44E−02 | 0.07 |
| DNTTIP1 | 10.12 | 1.07E−22 | 0.79 | SPNS1 | 5.32 | 7.64E−07 | 0.45 | RPUSD1 | 3.59 | 1.45E−03 | 1.13 | ELMOD2 | 2.55 | 3.50E−02 | −0.01 |
| WIPI1 | 10.09 | 1.39E−22 | 0.8 | ARRB1 | 5.32 | 7.34E−07 | 0.37 | RTP4 | 3.59 | 1.49E−03 | 1.08 | COPS2 | 2.55 | 3.47E−02 | −0.02 |
| IDS | 10.08 | 1.60E−22 | 0.83 | MAP3K3 | 5.32 | 7.54E−07 | 0.27 | CXCL14 | 3.59 | 1.49E−03 | 0.93 | RER1 | 2.55 | 3.44E−02 | −0.04 |
| IL32 | 10.07 | 1.83E−22 | 0.94 | ANKRD11 | 5.32 | 7.49E−07 | 0.17 | LSM3 | 3.59 | 1.48E−03 | 0.52 | ZNF592 | 2.55 | 3.45E−02 | −0.04 |
| HSPG2 | 10.07 | 1.74E−22 | 0.44 | UQCRHL | 5.31 | 8.07E−07 | 1.94 | ALG2 | 3.59 | 1.49E−03 | 0.45 | TBP | 2.55 | 3.48E−02 | −0.04 |
| SLC35F3 | 10.06 | 2.01E−22 | 0.64 | DSTN | 5.31 | 7.99E−07 | 0.26 | ZNF639 | 3.59 | 1.46E−03 | 0.4 | MAPKAPK2 | 2.55 | 3.44E−02 | −0.06 |
| NFE2L1 | 10.05 | 2.18E−22 | 0.95 | GNA11 | 5.31 | 8.08E−07 | 0.26 | FAM98A | 3.59 | 1.49E−03 | 0.4 | NPIPA5 | 2.54 | 3.53E−02 | 1.41 |
| ABLIM2 | 10.05 | 2.16E−22 | 0.73 | TXK | 5.3 | 8.22E−07 | 0.85 | SNAP47 | 3.59 | 1.46E−03 | 0.28 | COPZ2 | 2.54 | 3.57E−02 | 1.27 |
| RAB11A | 10.05 | 2.19E−22 | 0.46 | RFXANK | 5.3 | 8.19E−07 | 0.69 | PM20D2 | 3.59 | 1.45E−03 | 0.27 | CCDC102A | 2.54 | 3.57E−02 | 1.15 |
| GOLGA3 | 10.03 | 2.53E−22 | 0.55 | GMEB1 | 5.3 | 8.50E−07 | 0.42 | PIGX | 3.59 | 1.49E−03 | 0.24 | UBXN10 | 2.54 | 3.53E−02 | 0.62 |
| TMED2 | 10.02 | 2.80E−22 | 1.3 | SLC17A5 | 5.3 | 8.38E−07 | 0.24 | TCF7 | 3.59 | 1.47E−03 | 0.23 | C8orf37 | 2.54 | 3.56E−02 | 0.56 |
| GCNT1 | 10.02 | 3.01E−22 | 0.98 | CCDC34 | 5.29 | 8.92E−07 | 1.08 | PUS10 | 3.59 | 1.47E−03 | 0.18 | SLC7A2 | 2.54 | 3.58E−02 | 0.54 |
| CDR2 | 10.02 | 2.78E−22 | 0.4 | ATRAID | 5.29 | 8.73E−07 | 0.89 | HCG17 | 3.59 | 1.45E−03 | 0.16 | TMEM199 | 2.54 | 3.58E−02 | 0.51 |
| CTTN | 10.01 | 3.25E−22 | 0.39 | COL8A1 | 5.29 | 8.76E−07 | 0.88 | PWP1 | 3.59 | 1.46E−03 | 0.15 | ARSK | 2.54 | 3.57E−02 | 0.49 |
| RPS27 | 10 | 3.42E−22 | 0.86 | SCN9A | 5.29 | 8.68E−07 | 0.78 | SLC25A36 | 3.59 | 1.47E−03 | 0.13 | SIGMAR1 | 2.54 | 3.58E−02 | 0.47 |
| PIEZO1 | 10 | 3.58E−22 | 0.59 | PIP4K2C | 5.29 | 8.64E−07 | 0.46 | TMUB2 | 3.59 | 1.46E−03 | 0.12 | KLF11 | 2.54 | 3.56E−02 | 0.43 |
| VAV2 | 10 | 3.45E−22 | 0.48 | GNPTG | 5.29 | 8.89E−07 | 0.45 | RAB11FIP3 | 3.59 | 1.48E−03 | 0.12 | P3H1 | 2.54 | 3.57E−02 | 0.34 |
| AAK1 | 10 | 3.67E−22 | 0.44 | PRKCSH | 5.29 | 8.78E−07 | 0.37 | CTSC | 3.59 | 1.49E−03 | 0.12 | GAP43 | 2.54 | 3.54E−02 | 0.24 |
| SLC7A6 | 9.99 | 4.04E−22 | 0.87 | PEX26 | 5.29 | 8.80E−07 | 0.28 | SRGAP3 | 3.59 | 1.48E−03 | 0.11 | ASB6 | 2.54 | 3.60E−02 | 0.24 |
| PSMB7 | 9.99 | 4.01E−22 | 0.52 | KCNQ1OT1 | 5.29 | 8.86E−07 | 0.24 | ADSL | 3.58 | 1.54E−03 | 3.1 | LMNB2 | 2.54 | 3.55E−02 | 0.21 |
| ANKS1A | 9.99 | 3.95E−22 | 0.48 | FMN2 | 5.28 | 9.10E−07 | 1.35 | HECW1 | 3.58 | 1.54E−03 | 0.98 | MRPL47 | 2.54 | 3.60E−02 | 0.19 |
| LEMD1 | 9.98 | 4.28E−22 | 0.59 | ZNF493 | 5.28 | 9.13E−07 | 0.83 | ATP6VIE2 | 3.58 | 1.52E−03 | 0.85 | F2RL1 | 2.54 | 3.57E−02 | 0.13 |
| TSPAN13 | 9.96 | 5.11E−22 | 1.1 | ZNF865 | 5.28 | 9.06E−07 | 0.81 | TPGS1 | 3.58 | 1.55E−03 | 0.54 | GIPC1 | 2.54 | 3.53E−02 | 0.1 |
| AHDC1 | 9.94 | 6.20E−22 | 0.65 | KANK2 | 5.28 | 9.12E−07 | 0.45 | KMT5A | 3.58 | 1.51E−03 | 0.33 | CYB5D2 | 2.54 | 3.55E−02 | 0.1 |
| CYR61 | 9.93 | 6.94E−22 | 1.07 | MRPL42 | 5.28 | 9.19E−07 | 0.39 | ATP13A2 | 3.58 | 1.51E−03 | 0.24 | TUBA4A | 2.54 | 3.55E−02 | 0.05 |
| AUP1 | 9.93 | 6.76E−22 | 0.75 | ARFRPI | 5.28 | 9.24E−07 | 0.34 | HGD | 3.58 | 1.51E−03 | 0.21 | GOSR1 | 2.54 | 3.59E−02 | 0.05 |
| MOB3B | 9.91 | 8.52E−22 | 0.59 | CEBPZOS | 5.28 | 9.45E−07 | 0.27 | NOSIP | 3.58 | 1.54E−03 | 0.19 | BCAT1 | 2.54 | 3.52E−02 | −0.06 |
| PDXK | 9.91 | 8.50E−22 | 0.43 | MPZL3 | 5.28 | 9.11E−07 | 0.24 | EME2 | 3.58 | 1.53E−03 | 0.17 | STRIP1 | 2.54 | 3.61E−02 | −0.06 |
| COX7A2L | 9.9 | 9.61E−22 | 0.81 | DPP9 | 5.28 | 9.31E−07 | 0.19 | DCUN1D1 | 3.58 | 1.54E−03 | 0.13 | DGCR6 | 2.53 | 3.65E−02 | 7.28 |
| PHACTR3 | 9.89 | 1.05E−21 | 0.86 | PPP2R3A | 5.28 | 9.22E−07 | 0.18 | ARF4 | 3.58 | 1.50E−03 | 0.1 | ZNHIT2 | 2.53 | 3.69E−02 | 1.61 |
| ETV5 | 9.88 | 1.09E−21 | 1.05 | NOP2 | 5.27 | 9.77E−07 | 1.95 | MACROD1 | 3.58 | 1.54E−03 | 0.09 | DND1 | 2.53 | 3.68E−02 | 0.53 |
| EEF1D | 9.87 | 1.27E−21 | 0.58 | C12orf76 | 5.27 | 9.86E−07 | 0.7 | ADH1C | 3.57 | 1.57E−03 | 4.11 | PYURF | 2.53 | 3.68E−02 | 0.46 |
| FBN1 | 9.86 | 1.35E−21 | 1.27 | RNF157 | 5.27 | 9.68E−07 | 0.57 | SORCS1 | 3.57 | 1.60E−03 | 0.77 | GJC1 | 2.53 | 3.66E−02 | 0.42 |
| DYNLT1 | 9.85 | 1.49E−21 | 0.78 | SRSF9 | 5.27 | 9.53E−07 | 0.52 | SRF | 3.57 | 1.57E−03 | 0.68 | FAM200A | 2.53 | 3.66E−02 | 0.36 |
| NIPAL3 | 9.85 | 1.59E−21 | 0.72 | DPF2 | 5.27 | 9.75E−07 | 0.41 | MAN2B1 | 3.57 | 1.57E−03 | 0.42 | PHF13 | 2.53 | 3.66E−02 | 0.27 |
| C11orf54 | 9.83 | 1.89E−21 | 3.49 | AP2A1 | 5.27 | 9.75E−07 | 0.34 | SNX21 | 3.57 | 1.56E−03 | 0.34 | ERCC2 | 2.53 | 3.66E−02 | 0.26 |
| IL34 | 9.82 | 2.08E−21 | 2.31 | NSUN7 | 5.27 | 9.50E−07 | 0.28 | TPMT | 3.57 | 1.55E−03 | 0.2 | BUD13 | 2.53 | 3.61E−02 | 0.25 |
| KPNA6 | 9.81 | 2.34E−21 | 0.55 | ZFAND5 | 5.27 | 9.65E−07 | 0.23 | PLSCR4 | 3.57 | 1.55E−03 | 0.17 | LRRC75A | 2.53 | 3.69E−02 | 0.16 |
| CBWD5 | 9.81 | 2.29E−21 | 0.52 | ALMS1 | 5.27 | 9.64E−07 | 0.22 | C19orf12 | 3.57 | 1.58E−03 | 0.16 | PHAX | 2.53 | 3.68E−02 | 0.09 |
| GRAMD1A | 9.78 | 3.00E−21 | 0.52 | HMGN3 | 5.27 | 9.97E−07 | 0.18 | AGAP3 | 3.57 | 1.59E−03 | 0.15 | LANCL1 | 2.53 | 3.66E−02 | 0.08 |
| FNDC3B | 9.77 | 3.31E−21 | 0.11 | TXNRD3 | 5.26 | 1.01E−06 | 0.75 | CDK11B | 3.57 | 1.57E−03 | 0.13 | QSOX2 | 2.53 | 3.67E−02 | 0.05 |
| NMUR2 | 9.76 | 3.61E−21 | 1.84 | STS | 5.26 | 1.02E−06 | 0.68 | TNFRSF1A | 3.57 | 1.56E−03 | 0.12 | GBA | 2.53 | 3.68E−02 | 0.05 |
| CFH | 9.76 | 3.59E−21 | 0.72 | BCL7A | 5.26 | 1.00E−06 | 0.42 | ITGB5-AS1 | 3.57 | 1.59E−03 | 0.11 | HECTD3 | 2.53 | 3.68E−02 | 0.04 |
| ARHGAP42 | 9.76 | 3.80E−21 | 0.45 | ITM2B | 5.26 | 1.01E−06 | 0.38 | YIPF4 | 3.57 | 1.60E−03 | 0.1 | GNA13 | 2.53 | 3.69E−02 | 0.04 |
| PAFAH1B2 | 9.75 | 4.06E−21 | 0.56 | UNC45A | 5.26 | 1.01E−06 | 0.33 | SEH1L | 3.57 | 1.58E−03 | 0.09 | SLC44A3 | 2.53 | 3.69E−02 | 0.03 |
| MAGI2 | 9.75 | 4.24E−21 | 0.52 | TIPARP | 5.26 | 1.02E−06 | 0.27 | G6PC3 | 3.56 | 1.64E−03 | 1.07 | TMEM33 | 2.53 | 3.69E−02 | −0.01 |
| UQCRH | 9.74 | 4.43E−21 | 0.83 | LINC00665 | 5.25 | 1.06E−06 | 1.1 | SHMT2 | 3.56 | 1.64E−03 | 0.86 | UBE2Q2 | 2.53 | 3.69E−02 | −0.02 |
| DENND6B | 9.73 | 5.04E−21 | 0.88 | OAZ2 | 5.25 | 1.07E−06 | 0.65 | AFF3 | 3.56 | 1.64E−03 | 0.58 | WRB | 2.53 | 3.68E−02 | −0.04 |
| SH3PXD2B | 9.73 | 5.10E−21 | 0.55 | DIXDC1 | 5.25 | 1.07E−06 | 0.56 | MIR22HG | 3.56 | 1.65E−03 | 0.28 | SEC22B | 2.53 | 3.62E−02 | −0.06 |
| ATXN7 | 9.72 | 5.18E−21 | 0.74 | MBD3 | 5.25 | 1.06E−06 | 0.52 | SUGP1 | 3.56 | 1.63E−03 | 0.22 | IFNGR1 | 2.53 | 3.69E−02 | −0.13 |
| RNF7 | 9.71 | 6.20E−21 | 1.12 | DOCK8 | 5.25 | 1.09E−06 | 0.44 | NXF1 | 3.56 | 1.65E−03 | 0.19 | RASGRF1 | 2.52 | 3.75E−02 | 1.86 |
| TPI1 | 9.71 | 6.01E−21 | 0.76 | PTPN3 | 5.25 | 1.07E−06 | 0.25 | EZH1 | 3.56 | 1.62E−03 | 0.17 | SRRM3 | 2.52 | 3.76E−02 | 1.32 |
| BLCAP | 9.7 | 6.79E−21 | 0.52 | RHPN1 | 5.25 | 1.08E−06 | 0.24 | NACA | 3.56 | 1.62E−03 | 0.16 | SLC10A3 | 2.52 | 3.75E−02 | 0.65 |
| FXN | 9.68 | 7.69E−21 | 3.5 | TULP4 | 5.25 | 1.09E−06 | 0.24 | PNRC1 | 3.56 | 1.62E−03 | 0.11 | NME5 | 2.52 | 3.78E−02 | 0.53 |
| SFXN5 | 9.68 | 8.07E−21 | 0.89 | UBE20 | 5.25 | 1.09E−06 | 0.24 | UBE3D | 3.56 | 1.65E−03 | 0.09 | COL4A4 | 2.52 | 3.79E−02 | 0.37 |
| SPTAN1 | 9.68 | 8.09E−21 | 0.45 | SUMF2 | 5.25 | 1.05E−06 | 0.22 | PIK3AP1 | 3.56 | 1.63E−03 | 0 | MT-ND6 | 2.52 | 3.70E−02 | 0.35 |
| MALL | 9.67 | 8.32E−21 | 0.82 | CAV2 | 5.25 | 1.10E−06 | 0.19 | NCAM2 | 3.55 | 1.70E−03 | 1 | SLC39A6 | 2.52 | 3.79E−02 | 0.31 |
| EIF5B | 9.67 | 8.98E−21 | 0.46 | PCDH11Y | 5.24 | 1.14E−06 | 1.29 | IRF2BPL | 3.55 | 1.71E−03 | 0.85 | C12orf45 | 2.52 | 3.74E−02 | 0.17 |
| ARHGAP11B | 9.65 | 1.11E−20 | 3.06 | PEF1 | 5.24 | 1.15E−06 | 0.8 | GBP1 | 3.55 | 1.67E−03 | 0.67 | IGIP | 2.52 | 3.79E−02 | 0.12 |
| BSG | 9.65 | 1.06E−20 | 0.82 | PLAU | 5.24 | 1.12E−06 | 0.78 | STK16 | 3.55 | 1.67E−03 | 0.61 | SPRTN | 2.52 | 3.74E−02 | 0.1 |
| COL3A1 | 9.65 | 1.10E−20 | 0.47 | LRRC20 | 5.24 | 1.15E−06 | 0.76 | MRPL37 | 3.55 | 1.68E−03 | 0.52 | ADGRG1 | 2.52 | 3.73E−02 | 0.09 |
| ATP2B1 | 9.64 | 1.17E−20 | 0.62 | TIMM13 | 5.24 | 1.12E−06 | 0.58 | NDUFA4 | 3.55 | 1.68E−03 | 0.36 | CPPED1 | 2.52 | 3.79E−02 | 0.04 |
| ISPD | 9.64 | 1.16E−20 | 0.58 | TMEM127 | 5.24 | 1.16E−06 | 0.53 | ATL1 | 3.55 | 1.67E−03 | 0.29 | TRIP6 | 2.52 | 3.77E−02 | −0.02 |
| SERINC2 | 9.64 | 1.17E−20 | 0.58 | IRAK1BP1 | 5.24 | 1.16E−06 | 0.44 | CHTF8 | 3.55 | 1.70E−03 | 0.24 | AGO1 | 2.52 | 3.73E−02 | −0.07 |
| TMED9 | 9.63 | 1.26E−20 | 1.17 | GOPC | 5.24 | 1.15E−06 | 0.31 | NIPAL1 | 3.55 | 1.67E−03 | 0.21 | GMEB2 | 2.52 | 3.79E−02 | −0.08 |
| NDUFS6 | 9.63 | 1.31E−20 | 1.02 | EEF2K | 5.24 | 1.15E−06 | 0.25 | COX7B | 3.55 | 1.70E−03 | 0.18 | USF2 | 2.52 | 3.80E−02 | −0.09 |
| PRR13 | 9.63 | 1.25E−20 | 0.84 | COL16A1 | 5.24 | 1.11E−06 | 0.24 | ZNF341 | 3.55 | 1.68E−03 | 0.17 | HOTAIRM1 | 2.51 | 3.88E−02 | 0.89 |
| MYL12A | 9.63 | 1.25E−20 | 0.64 | MORF4L1 | 5.24 | 1.16E−06 | 0.23 | ATP2C1 | 3.55 | 1.70E−03 | 0.14 | DHCR7 | 2.51 | 3.85E−02 | 0.73 |
| DMGDH | 9.62 | 1.37E−20 | 1.65 | SLC17A9 | 5.24 | 1.12E−06 | 0.22 | LPIN3 | 3.55 | 1.70E−03 | 0.13 | LINC01098 | 2.51 | 3.81E−02 | 0.59 |
| FAM50A | 9.62 | 1.45E−20 | 0.78 | JAK3 | 5.23 | 1.18E−06 | 0.85 | SMYD4 | 3.55 | 1.68E−03 | 0.11 | FRMD6-AS2 | 2.51 | 3.85E−02 | 0.57 |
| SIPA1L3 | 9.62 | 1.43E−20 | 0.34 | EVA1A | 5.23 | 1.18E−06 | 0.82 | AAMDC | 3.55 | 1.67E−03 | 0.09 | TMEM92-AS1 | 2.51 | 3.83E−02 | 0.56 |
| PSMB9 | 9.61 | 1.47E−20 | 1.44 | SLC35B1 | 5.23 | 1.22E−06 | 0.68 | PLEKHA7 | 3.55 | 1.71E−03 | 0.09 | HSD3B7 | 2.51 | 3.85E−02 | 0.31 |
| PURA | 9.6 | 1.62E−20 | 0.54 | ERGIC3 | 5.23 | 1.22E−06 | 0.25 | DHRS12 | 3.54 | 1.73E−03 | 2.79 | CRIPT | 2.51 | 3.84E−02 | 0.29 |
| ROM01 | 9.59 | 1.92E−20 | 1.64 | FGF1 | 5.22 | 1.25E−06 | 1.36 | ZFPL1 | 3.54 | 1.73E−03 | 0.52 | CDC26 | 2.51 | 3.83E−02 | 0.26 |
| OAS1 | 9.59 | 1.83E−20 | 1.16 | RYR3 | 5.22 | 1.29E−06 | 1.24 | DNAJC4 | 3.54 | 1.75E−03 | 0.29 | INPP5K | 2.51 | 3.81E−02 | 0.08 |
| CCPG1 | 9.59 | 1.89E−20 | 0.56 | ATG101 | 5.22 | 1.25E−06 | 0.85 | HAX1 | 3.54 | 1.74E−03 | 0.27 | FBXO21 | 2.51 | 3.85E−02 | 0.01 |
| TMEM241 | 9.59 | 1.81E−20 | 0.47 | FAH | 5.22 | 1.26E−06 | 0.72 | ENOX1 | 3.54 | 1.77E−03 | 0.24 | ATXN2L | 2.51 | 3.88E−02 | 0.01 |
| CAMK2G | 9.58 | 2.10E−20 | 0.49 | HSBP1L1 | 5.22 | 1.23E−06 | 0.55 | HNF4G | 3.54 | 1.77E−03 | 0.18 | ARAP1 | 2.51 | 3.83E−02 | −0.02 |
| CYB5B | 9.56 | 2.47E−20 | 0.56 | IDH2 | 5.22 | 1.25E−06 | 0.54 | MYNN | 3.54 | 1.72E−03 | 0.13 | HCFC1 | 2.51 | 3.86E−02 | −0.03 |
| PDE11A | 9.53 | 3.15E−20 | 2.17 | B4GALT2 | 5.22 | 1.28E−06 | 0.5 | DNAJB12 | 3.54 | 1.77E−03 | 0.13 | PGGT1B | 2.51 | 3.88E−02 | −0.06 |
| CNTN5 | 9.53 | 3.27E−20 | 1.44 | FAM219B | 5.22 | 1.27E−06 | 0.38 | HDGF | 3.54 | 1.77E−03 | 0.07 | GPI | 2.51 | 3.86E−02 | −0.07 |
| WDR45 | 9.51 | 3.90E−20 | 0.69 | AK2 | 5.22 | 1.24E−06 | 0.19 | TMEM178B | 3.54 | 1.77E−03 | −0.05 | LRTOMT | 2.5 | 3.99E−02 | 3.99 |
| TNIP1 | 9.51 | 4.08E−20 | 0.49 | PDZRN4 | 5.21 | 1.34E−06 | 2.51 | CTSL | 3.53 | 1.83E−03 | 0.68 | PTGDS | 2.5 | 3.94E−02 | 1.74 |
| ZNF75A | 9.48 | 5.40E−20 | 0.77 | PYCR1 | 5.21 | 1.35E−06 | 1.12 | TLE2 | 3.53 | 1.79E−03 | 0.52 | NIM1K | 2.5 | 3.93E−02 | 1.35 |
| MRC2 | 9.47 | 5.63E−20 | 1.1 | MKKS | 5.21 | 1.36E−06 | 0.76 | RNF152 | 3.53 | 1.82E−03 | 0.34 | TRPV1 | 2.5 | 3.94E−02 | 0.71 |
| MARVELD1 | 9.45 | 7.13E−20 | 1.04 | DPM2 | 5.21 | 1.31E−06 | 0.46 | HRAS | 3.53 | 1.80E−03 | 0.23 | ASB3 | 2.5 | 3.94E−02 | 0.51 |
| OGFR | 9.45 | 7.14E−20 | 0.7 | ZRSR2 | 5.21 | 1.34E−06 | 0.46 | MLX | 3.53 | 1.80E−03 | 0.17 | VBP1 | 2.5 | 3.90E−02 | 0.19 |
| LINC00886 | 9.45 | 6.62E−20 | 0.63 | PRICKLE2 | 5.21 | 1.29E−06 | 0.29 | YIPF1 | 3.53 | 1.84E−03 | 0.17 | RNF141 | 2.5 | 3.98E−02 | 0.09 |
| TERF2IP | 9.45 | 7.23E−20 | 0.55 | TRADD | 5.21 | 1.32E−06 | 0.29 | CREM | 3.53 | 1.79E−03 | 0.07 | DDX43 | 2.5 | 3.95E−02 | 0.07 |
| GCG | 9.44 | 7.33E−20 | 3.11 | RXRA | 5.21 | 1.35E−06 | 0.26 | SEMA3F-AS1 | 3.52 | 1.87E−03 | 1.39 | TADA3 | 2.5 | 3.95E−02 | 0.06 |
| WNT2B | 9.44 | 7.79E−20 | 0.94 | AGAP4 | 5.21 | 1.30E−06 | 0.23 | SF3A1 | 3.52 | 1.90E−03 | 0.54 | RGS3 | 2.5 | 3.90E−02 | 0.05 |
| IFIT3 | 9.43 | 8.35E−20 | 2.34 | ORC3 | 5.21 | 1.32E−06 | 0.23 | PREB | 3.52 | 1.85E−03 | 0.49 | TATDN1 | 2.5 | 3.98E−02 | 0.04 |
| SRPRB | 9.41 | 9.68E−20 | 1.17 | MAP7D1 | 5.21 | 1.33E−06 | 0.2 | KPNA2 | 3.52 | 1.85E−03 | 0.36 | SLC30A1 | 2.5 | 3.90E−02 | 0.02 |
| FUT2 | 9.41 | 1.02E−19 | 0.99 | RRAD | 5.2 | 1.40E−06 | 1.4 | TSR1 | 3.52 | 1.85E−03 | 0.28 | LPCAT3 | 2.5 | 3.93E−02 | −0.01 |
| ITGBL1 | 9.41 | 9.78E−20 | 0.95 | TCF4 | 5.2 | 1.41E−06 | 0.44 | VPS52 | 3.52 | 1.87E−03 | 0.12 | PDPR | 2.5 | 3.97E−02 | −0.02 |
| CLSTN1 | 9.4 | 1.10E−19 | 0.46 | OSBPL11 | 5.2 | 1.42E−06 | 0.31 | SEC24C | 3.52 | 1.89E−03 | 0.09 | NEGR1 | 2.5 | 3.92E−02 | −0.04 |
| LOXL4 | 9.39 | 1.18E−19 | 1.61 | NADSYN1 | 5.2 | 1.38E−06 | 0.25 | DNAJC18 | 3.51 | 1.92E−03 | 1.1 | NCOA4 | 2.5 | 3.91E−02 | −0.06 |
| NUAK1 | 9.38 | 1.38E−19 | 1.23 | PSMB1 | 5.2 | 1.41E−06 | 0.24 | KCNAB2 | 3.51 | 1.96E−03 | 0.62 | COLGALT1 | 2.5 | 3.92E−02 | −0.07 |
| FKBP8 | 9.38 | 1.32E−19 | 0.83 | CARS2 | 5.2 | 1.41E−06 | 0.23 | FBXL15 | 3.51 | 1.95E−03 | 0.38 | GGA1 | 2.5 | 3.94E−02 | −0.07 |
| PHLPP2 | 9.38 | 1.35E−19 | 0.76 | RHBDL2 | 5.2 | 1.40E−06 | 0.16 | CHN1 | 3.51 | 1.94E−03 | 0.27 | CHST4 | 2.49 | 4.00E−02 | 0.46 |
| PIGQ | 9.35 | 1.75E−19 | 0.86 | YIF1A | 5.19 | 1.44E−06 | 0.66 | CFAP221 | 3.51 | 1.95E−03 | 0.17 | HLA-DRB1 | 2.49 | 4.04E−02 | 0.39 |
| MLEC | 9.35 | 1.82E−19 | 0.78 | GAS7 | 5.19 | 1.44E−06 | 0.64 | ZBTB7A | 3.51 | 1.98E−03 | 0.11 | SPRYD3 | 2.49 | 4.08E−02 | 0.22 |
| SAMD4B | 9.35 | 1.73E−19 | 0.51 | NXNL2 | 5.18 | 1.54E−06 | 1.41 | PTBP1 | 3.51 | 1.92E−03 | 0.09 | ATG4A | 2.49 | 4.01E−02 | 0.18 |
| CAPG | 9.34 | 1.94E−19 | 0.93 | USP18 | 5.18 | 1.51E−06 | 1.36 | ADCY7 | 3.51 | 1.93E−03 | 0.08 | ACAP2 | 2.49 | 4.06E−02 | 0.1 |
| ARL5A | 9.34 | 1.94E−19 | 0.85 | PPP1R9B | 5.18 | 1.54E−06 | 1.2 | LMAN2L | 3.51 | 1.98E−03 | 0.05 | FAM213A | 2.49 | 4.08E−02 | 0.06 |
| SHARPIN | 9.34 | 1.85E−19 | 0.73 | SLC2A1-AS1 | 5.18 | 1.55E−06 | 0.83 | KCNQ3 | 3.5 | 2.03E−03 | 0.69 | OSBPL7 | 2.49 | 4.08E−02 | 0.03 |
| ITGB5 | 9.34 | 1.95E−19 | 0.44 | GUCD1 | 5.18 | 1.54E−06 | 0.77 | GGT7 | 3.5 | 1.99E−03 | 0.16 | PPP1CC | 2.49 | 4.03E−02 | 0 |
| LINC01605 | 9.33 | 2.03E−19 | 2.03 | RABL2B | 5.18 | 1.54E−06 | 0.48 | CPSF3 | 3.5 | 2.05E−03 | 0.16 | ZDHHC16 | 2.49 | 4.04E−02 | −0.06 |
| MMP7 | 9.33 | 2.17E−19 | 0.78 | TNNC2 | 5.17 | 1.62E−06 | 2.81 | FAM192A | 3.5 | 1.98E−03 | 0.12 | PIK3CD | 2.48 | 4.17E−02 | 0.67 |
| MECP2 | 9.32 | 2.28E−19 | 0.56 | MIR646HG | 5.17 | 1.66E−06 | 1.26 | UBE2J2 | 3.5 | 2.01E−03 | 0.1 | EFCAB10 | 2.48 | 4.13E−02 | 0.61 |
| NHSL2 | 9.31 | 2.64E−19 | 1.71 | SNAP29 | 5.17 | 1.63E−06 | 0.43 | RBBP4 | 3.5 | 1.99E−03 | 0.09 | SERPINB9P1 | 2.48 | 4.14E−02 | 0.33 |
| OFD1 | 9.31 | 2.59E−19 | 0.45 | ANKRD6 | 5.17 | 1.65E−06 | 0.25 | YTHDF2 | 3.5 | 2.01E−03 | 0.06 | COG8 | 2.48 | 4.17E−02 | 0.28 |
| VCP | 9.3 | 2.73E−19 | 0.5 | SHANK2 | 5.17 | 1.60E−06 | 0.21 | AMD1 | 3.5 | 1.98E−03 | 0.01 | TYSND1 | 2.48 | 4.12E−02 | 0.18 |
| PDLIM1 | 9.3 | 2.80E−19 | 0.47 | E2F3 | 5.17 | 1.65E−06 | 0.18 | ARL4D | 3.49 | 2.12E−03 | 1.67 | ZDHHC23 | 2.48 | 4.14E−02 | 0.14 |
| XRN1 | 9.3 | 2.86E−19 | 0.41 | GCLM | 5.16 | 1.71E−06 | 0.51 | ZDHHC4 | 3.49 | 2.11E−03 | 0.4 | RAD9A | 2.48 | 4.10E−02 | 0.1 |
| SLC8B1 | 9.29 | 3.07E−19 | 0.84 | GSS | 5.16 | 1.70E−06 | 0.29 | COPS6 | 3.49 | 2.07E−03 | 0.26 | TMEM45B | 2.48 | 4.18E−02 | 0.09 |
| CHRM2 | 9.28 | 3.18E−19 | 1.92 | MAD1L1 | 5.16 | 1.69E−06 | 0.24 | VIM | 3.49 | 2.09E−03 | 0.2 | GRB7 | 2.48 | 4.16E−02 | 0.07 |
| DNAJA3 | 9.27 | 3.59E−19 | 0.66 | CCDC57 | 5.16 | 1.75E−06 | 0.22 | PPFIBP1 | 3.49 | 2.06E−03 | 0.16 | VAMP7 | 2.48 | 4.15E−02 | 0.06 |
| PPIA | 9.27 | 3.73E−19 | 0.61 | ZNF791 | 5.16 | 1.73E−06 | 0.2 | EMP2 | 3.49 | 2.11E−03 | 0.16 | NEDD1 | 2.48 | 4.15E−02 | 0.05 |
| HES1 | 9.26 | 4.15E−19 | 0.7 | POLD4 | 5.15 | 1.84E−06 | 1.24 | PTGFRN | 3.49 | 2.09E−03 | 0.14 | PDCD5 | 2.48 | 4.19E−02 | 0.05 |
| MAPKBP1 | 9.26 | 3.93E−19 | 0.67 | PRKACA | 5.15 | 1.82E−06 | 0.64 | FAM185A | 3.49 | 2.09E−03 | 0.13 | MT1M | 2.47 | 4.24E−02 | 1.55 |
| ZXDC | 9.26 | 3.96E−19 | 0.51 | ZFP30 | 5.15 | 1.76E−06 | 0.51 | ARHGAP35 | 3.49 | 2.09E−03 | 0.12 | SYP | 2.47 | 4.23E−02 | 1.06 |
| C19orf53 | 9.25 | 4.54E−19 | 1.51 | CDC42EP1 | 5.15 | 1.83E−06 | 0.51 | EXOC7 | 3.49 | 2.07E−03 | 0.1 | MIS18A | 2.47 | 4.22E−02 | 0.6 |
| RPS7 | 9.24 | 4.77E−19 | 0.68 | ATG2A | 5.15 | 1.77E−06 | 0.38 | JMY | 3.49 | 2.10E−03 | 0.09 | MTTP | 2.47 | 4.22E−02 | 0.48 |
| CAPZB | 9.24 | 4.82E−19 | 0.4 | HMGXB4 | 5.15 | 1.77E−06 | 0.22 | DBNL | 3.49 | 2.10E−03 | 0.07 | SPATA2 | 2.47 | 4.31E−02 | 0.32 |
| GPS2 | 9.23 | 5.08E−19 | 1.29 | FOXK1 | 5.15 | 1.79E−06 | 0.22 | ZNF814 | 3.49 | 2.08E−03 | 0.06 | ZMYND19 | 2.47 | 4.31E−02 | 0.28 |
| DPYSL3 | 9.23 | 5.15E−19 | 1.03 | PCDH1 | 5.15 | 1.83E−06 | 0.22 | TBRG1 | 3.49 | 2.06E−03 | 0.04 | TOR1A | 2.47 | 4.22E−02 | 0.2 |
| TMEM263 | 9.22 | 5.49E−19 | 0.63 | GFER | 5.14 | 1.93E−06 | 1.3 | PABPCIL | 3.49 | 2.08E−03 | 0.04 | TMEM116 | 2.47 | 4.25E−02 | 0.18 |
| XDH | 9.22 | 5.75E−19 | 0.59 | COPRS | 5.14 | 1.87E−06 | 1.16 | GPX3 | 3.48 | 2.16E−03 | 1.99 | SERHL2 | 2.47 | 4.27E−02 | 0.13 |
| ANKRD27 | 9.2 | 6.57E−19 | 0.52 | C4orf47 | 5.14 | 1.94E−06 | 0.63 | DIRC3 | 3.48 | 2.16E−03 | 0.73 | HADH | 2.47 | 4.23E−02 | 0.11 |
| MCC | 9.2 | 6.63E−19 | 0.47 | HYDIN | 5.14 | 1.88E−06 | 0.49 | MRPS15 | 3.48 | 2.17E−03 | 0.56 | CDHR5 | 2.47 | 4.25E−02 | 0.11 |
| EIF3K | 9.19 | 7.71E−19 | 0.67 | GTF2H2 | 5.14 | 1.93E−06 | 0.47 | PLSCR2 | 3.48 | 2.15E−03 | 0.52 | GDPD5 | 2.47 | 4.21E−02 | 0.09 |
| FTO | 9.18 | 8.41E−19 | 0.36 | SRA1 | 5.14 | 1.89E−06 | 0.33 | NKIRAS2 | 3.48 | 2.19E−03 | 0.47 | GLE1 | 2.47 | 4.31E−02 | 0.05 |
| DNAJC7 | 9.17 | 9.25E−19 | 0.44 | ARPP19 | 5.14 | 1.92E−06 | 0.25 | HNRNPAO | 3.48 | 2.19E−03 | 0.17 | NDUFA10 | 2.47 | 4.23E−02 | 0.03 |
| DCDC1 | 9.16 | 9.58E−19 | 1.13 | APOO | 5.14 | 1.94E−06 | 0.22 | CLTC | 3.48 | 2.18E−03 | 0.16 | NQO2 | 2.47 | 4.27E−02 | 0.01 |
| LINC00685 | 9.15 | 1.04E−18 | 1.3 | CFAP43 | 5.13 | 1.99E−06 | 0.77 | NPAT | 3.48 | 2.17E−03 | 0.09 | GID8 | 2.47 | 4.30E−02 | 0 |
| LAMP1 | 9.15 | 1.10E−18 | 0.74 | ZNF331 | 5.13 | 1.96E−06 | 0.71 | IGF2R | 3.48 | 2.16E−03 | 0.08 | AC008074.1 | 2.47 | 4.22E−02 | −0.01 |
| TRAM1 | 9.15 | 1.05E−18 | 0.54 | TXN2 | 5.13 | 1.97E−06 | 0.63 | HEBP2 | 3.48 | 2.18E−03 | 0 | GNG12-AS1 | 2.47 | 4.25E−02 | −0.01 |
| FAM114A1 | 9.15 | 1.08E−18 | 0.48 | PDP2 | 5.13 | 2.02E−06 | 0.48 | SCUBE3 | 3.47 | 2.23E−03 | 1.41 | COQ9 | 2.47 | 4.27E−02 | −0.01 |
| KNOP1 | 9.14 | 1.14E−18 | 0.93 | FXYD5 | 5.13 | 2.03E−06 | 0.39 | OGFOD2 | 3.47 | 2.20E−03 | 0.87 | CROCC | 2.47 | 4.25E−02 | −0.03 |
| ODF2 | 9.14 | 1.22E−18 | 0.74 | DRG1 | 5.13 | 2.00E−06 | 0.28 | NEURL1 | 3.47 | 2.22E−03 | 0.85 | GCC2-AS1 | 2.47 | 4.31E−02 | −0.04 |
| IFT81 | 9.13 | 1.31E−18 | 0.73 | WDPCP | 5.13 | 2.04E−06 | 0.24 | AOC1 | 3.47 | 2.24E−03 | 0.78 | MED28 | 2.47 | 4.24E−02 | −0.12 |
| CA13 | 9.13 | 1.28E−18 | 0.68 | KCNAB1 | 5.12 | 2.11E−06 | 1.12 | STMN3 | 3.47 | 2.25E−03 | 0.6 | AP3B2 | 2.46 | 4.33E−02 | 1 |
| PLSCR1 | 9.13 | 1.25E−18 | 0.48 | RAB11FIP5 | 5.12 | 2.10E−06 | 0.72 | PGD | 3.47 | 2.27E−03 | 0.44 | ALKBH2 | 2.46 | 4.38E−02 | 0.96 |
| ARPC3 | 9.13 | 1.35E−18 | 0.47 | EIF3I | 5.12 | 2.05E−06 | 0.53 | DEF6 | 3.47 | 2.26E−03 | 0.27 | PALM | 2.46 | 4.33E−02 | 0.95 |
| STEAP1B | 9.12 | 1.37E−18 | 0.89 | INPP5B | 5.12 | 2.11E−06 | 0.32 | EPPK1 | 3.47 | 2.20E−03 | 0.22 | CDH23 | 2.46 | 4.36E−02 | 0.66 |
| CST3 | 9.11 | 1.58E−18 | 0.88 | NME7 | 5.12 | 2.11E−06 | 0.21 | FAM200B | 3.47 | 2.23E−03 | 0.2 | ATP8B3 | 2.46 | 4.37E−02 | 0.56 |
| GSTP1 | 9.11 | 1.48E−18 | 0.56 | AZIN2 | 5.11 | 2.21E−06 | 0.96 | CIAPIN1 | 3.47 | 2.23E−03 | 0.18 | NTHL1 | 2.46 | 4.33E−02 | 0.53 |
| DZIP1L | 9.1 | 1.70E−18 | 1.48 | JDP2 | 5.11 | 2.18E−06 | 0.56 | ADIPOR1 | 3.47 | 2.25E−03 | 0.18 | USE1 | 2.46 | 4.36E−02 | 0.3 |
| CEACAM1 | 9.1 | 1.68E−18 | 0.7 | COA4 | 5.11 | 2.26E−06 | 0.53 | CLCN6 | 3.47 | 2.25E−03 | 0.14 | AIMP2 | 2.46 | 4.40E−02 | 0.11 |
| SLC5A1 | 9.09 | 1.78E−18 | 1.01 | ADAT1 | 5.11 | 2.19E−06 | 0.5 | MYO5A | 3.47 | 2.23E−03 | 0.12 | DPH5 | 2.46 | 4.41E−02 | 0.1 |
| GARS | 9.08 | 1.95E−18 | 0.64 | TMEM230 | 5.11 | 2.23E−06 | 0.33 | HHAT | 3.47 | 2.24E−03 | 0.12 | ARL6 | 2.46 | 4.42E−02 | 0.1 |
| WBP2 | 9.08 | 2.11E−18 | 0.61 | TRAK2 | 5.11 | 2.20E−06 | 0.24 | NBPF19 | 3.47 | 2.23E−03 | 0.11 | GNA15 | 2.46 | 4.31E−02 | 0.09 |
| NOL3 | 9.08 | 2.09E−18 | 0.59 | PDE4C | 5.1 | 2.29E−06 | 1.14 | COG7 | 3.47 | 2.23E−03 | 0.1 | ATXN7L3 | 2.46 | 4.34E−02 | 0.05 |
| PRDX1 | 9.07 | 2.24E−18 | 0.98 | HEBP1 | 5.1 | 2.37E−06 | 0.71 | QRSL1 | 3.47 | 2.19E−03 | 0.06 | SLC25A12 | 2.46 | 4.31E−02 | 0.02 |
| UBALD2 | 9.07 | 2.15E−18 | 0.6 | TRAPPC6A | 5.1 | 2.28E−06 | 0.59 | TRAF3IP2-AS1 | 3.47 | 2.26E−03 | 0.05 | PCCB | 2.46 | 4.34E−02 | 0.01 |
| HES4 | 9.06 | 2.49E−18 | 0.69 | MRPL19 | 5.1 | 2.34E−06 | 0.38 | C7orf61 | 3.46 | 2.32E−03 | 1.26 | AP4E1 | 2.46 | 4.34E−02 | −0.01 |
| QTRT1 | 9.06 | 2.32E−18 | 0.63 | ZDHHC9 | 5.1 | 2.30E−06 | 0.32 | C20orf27 | 3.46 | 2.30E−03 | 1.06 | MKNK2 | 2.46 | 4.35E−02 | −0.08 |
| NMD3 | 9.06 | 2.33E−18 | 0.48 | PIN4 | 5.1 | 2.30E−06 | 0.29 | SERPING1 | 3.46 | 2.28E−03 | 0.71 | TPM2 | 2.46 | 4.35E−02 | −0.2 |
| SUPT5H | 9.06 | 2.50E−18 | 0.48 | SYMPK | 5.1 | 2.37E−06 | 0.24 | CPE | 3.46 | 2.32E−03 | 0.71 | POU5F1B | 2.45 | 4.50E−02 | 2.94 |
| CHCHD6 | 9.04 | 2.86E−18 | 0.69 | YDJC | 5.09 | 2.45E−06 | 1.03 | NFKBIL1 | 3.46 | 2.28E−03 | 0.64 | EPX | 2.45 | 4.53E−02 | 2.27 |
| ATOX1 | 9.03 | 3.19E−18 | 0.83 | RPGRIPIL | 5.09 | 2.47E−06 | 0.49 | BCKDK | 3.46 | 2.33E−03 | 0.54 | PLET1 | 2.45 | 4.50E−02 | 1.96 |
| TSPAN5 | 9.03 | 3.08E−18 | 0.47 | LCA5 | 5.09 | 2.48E−06 | 0.46 | C4orf48 | 3.46 | 2.34E−03 | 0.46 | NELL1 | 2.45 | 4.50E−02 | 1.16 |
| PCDH7 | 9.03 | 3.27E−18 | 0.43 | ZBTB46 | 5.09 | 2.50E−06 | 0.41 | AGTRAP | 3.46 | 2.28E−03 | 0.4 | C16orf91 | 2.45 | 4.53E−02 | 1.03 |
| MFSD14A | 9 | 4.28E−18 | 2.3 | PBX2 | 5.09 | 2.48E−06 | 0.37 | MTSS1L | 3.46 | 2.28E−03 | 0.32 | AC024084.1 | 2.45 | 4.53E−02 | 0.82 |
| CDKL1 | 9 | 4.06E−18 | 0.83 | MTMR1 | 5.09 | 2.41E−06 | 0.27 | FAS | 3.46 | 2.28E−03 | 0.26 | GHDC | 2.45 | 4.43E−02 | 0.43 |
| ZHX3 | 9 | 4.13E−18 | 0.67 | LPIN2 | 5.09 | 2.44E−06 | 0.23 | PECR | 3.46 | 2.33E−03 | 0.23 | ADAMTS17 | 2.45 | 4.50E−02 | 0.36 |
| TXN | 9 | 4.20E−18 | 0.67 | ROBO1 | 5.09 | 2.47E−06 | 0.13 | CAV1 | 3.46 | 2.32E−03 | 0.22 | CLPP | 2.45 | 4.44E−02 | 0.32 |
| HOOK2 | 8.99 | 4.65E−18 | 0.41 | STARD9 | 5.08 | 2.56E−06 | 1.03 | MRPL4 | 3.46 | 2.30E−03 | 0.2 | ZNF14 | 2.45 | 4.47E−02 | 0.19 |
| CST6 | 8.98 | 4.90E−18 | 3.2 | GATA3 | 5.08 | 2.59E−06 | 0.82 | TARS | 3.46 | 2.27E−03 | 0.11 | FBXO41 | 2.45 | 4.42E−02 | 0.1 |
| CSRP1 | 8.98 | 5.13E−18 | 1.14 | FAM210B | 5.08 | 2.52E−06 | 0.77 | FGF2 | 3.46 | 2.34E−03 | −0.01 | DTNBP1 | 2.45 | 4.43E−02 | 0.04 |
| DNAJB2 | 8.98 | 4.96E−18 | 0.78 | AKAP12 | 5.08 | 2.57E−06 | 0.44 | B3GALT6 | 3.45 | 2.43E−03 | 1.51 | SAMD12-AS1 | 2.45 | 4.43E−02 | 0 |
| MPG | 8.98 | 4.75E−18 | 0.7 | FAM83H | 5.08 | 2.61E−06 | 0.34 | WDR54 | 3.45 | 2.41E−03 | 0.95 | DTWD1 | 2.45 | 4.51E−02 | 0 |
| CLIC1 | 8.98 | 5.03E−18 | 0.59 | RAB11B | 5.08 | 2.56E−06 | 0.27 | CLIC2 | 3.45 | 2.37E−03 | 0.89 | AFG3L2 | 2.45 | 4.52E−02 | −0.03 |
| PRKAG1 | 8.97 | 5.26E−18 | 0.67 | TTF1 | 5.08 | 2.60E−06 | 0.25 | PRELID3A | 3.45 | 2.35E−03 | 0.85 | PNPLA3 | 2.45 | 4.51E−02 | −0.1 |
| DMKN | 8.97 | 5.53E−18 | 0.53 | CEP112 | 5.08 | 2.56E−06 | 0.24 | ACP2 | 3.45 | 2.42E−03 | 0.83 | QPCT | 2.44 | 4.63E−02 | 1.48 |
| DHRSX | 8.97 | 5.21E−18 | 0.44 | MCOLN3 | 5.07 | 2.66E−06 | 1.7 | CHCHD1 | 3.45 | 2.37E−03 | 0.56 | SPNS3 | 2.44 | 4.60E−02 | 1.3 |
| PALLD | 8.97 | 5.42E−18 | 0.4 | CIC | 5.07 | 2.68E−06 | 0.54 | GTF3C6 | 3.45 | 2.40E−03 | 0.56 | CD81-AS1 | 2.44 | 4.54E−02 | 1.28 |
| COX5B | 8.96 | 5.85E−18 | 1.13 | PEX11B | 5.07 | 2.63E−06 | 0.39 | BAHD1 | 3.45 | 2.38E−03 | 0.37 | ZNF628 | 2.44 | 4.58E−02 | 1.16 |
| IQSEC2 | 8.96 | 5.88E−18 | 0.82 | EIF4E2 | 5.07 | 2.65E−06 | 0.18 | AAAS | 3.45 | 2.37E−03 | 0.28 | VAT1L | 2.44 | 4.60E−02 | 0.83 |
| SLC12A7 | 8.96 | 6.09E−18 | 0.47 | CFAP47 | 5.06 | 2.88E−06 | 1.27 | GPN2 | 3.45 | 2.39E−03 | 0.24 | ERG | 2.44 | 4.59E−02 | 0.45 |
| CBX6 | 8.95 | 6.68E−18 | 1.38 | PILRA | 5.06 | 2.88E−06 | 0.69 | NRAS | 3.45 | 2.41E−03 | 0.2 | TRMT6 | 2.44 | 4.64E−02 | 0.28 |
| MAFG | 8.95 | 6.55E−18 | 1.14 | SOD1 | 5.06 | 2.76E−06 | 0.39 | PARM1 | 3.45 | 2.42E−03 | 0.18 | PATZ1 | 2.44 | 4.58E−02 | 0.11 |
| ZBTB16 | 8.94 | 7.09E−18 | 1.1 | SLC25A39 | 5.06 | 2.84E−06 | 0.31 | CTSS | 3.45 | 2.35E−03 | 0.14 | ENKD1 | 2.44 | 4.63E−02 | 0.11 |
| BANF1 | 8.93 | 7.91E−18 | 1.41 | STIP1 | 5.06 | 2.78E−06 | 0.29 | NDE1 | 3.45 | 2.36E−03 | 0.1 | VAPA | 2.44 | 4.55E−02 | 0.09 |
| PAWR | 8.93 | 7.86E−18 | 0.39 | NUPL2 | 5.06 | 2.89E−06 | 0.25 | DCUN1D4 | 3.45 | 2.36E−03 | 0.1 | ZNF484 | 2.44 | 4.59E−02 | 0.08 |
| NDUFS8 | 8.92 | 8.45E−18 | 0.92 | ELK3 | 5.06 | 2.84E−06 | 0.24 | NFYA | 3.45 | 2.37E−03 | 0.02 | NT5C | 2.44 | 4.59E−02 | 0.04 |
| COTL1 | 8.92 | 8.63E−18 | 0.69 | C4orf19 | 5.06 | 2.89E−06 | 0.24 | CLPSL1 | 3.44 | 2.47E−03 | 2.47 | CLDN12 | 2.44 | 4.55E−02 | 0.02 |
| ZNF37A | 8.91 | 9.00E−18 | 3.64 | CTBP1 | 5.06 | 2.88E−06 | 0.23 | METRNL | 3.44 | 2.48E−03 | 0.73 | XPOT | 2.44 | 4.56E−02 | 0.02 |
| RAB26 | 8.91 | 9.02E−18 | 1.42 | CASC15 | 5.06 | 2.82E−06 | 0.2 | POLE3 | 3.44 | 2.44E−03 | 0.63 | OCRL | 2.44 | 4.62E−02 | 0.01 |
| RPS5 | 8.91 | 8.92E−18 | 0.86 | C2orf54 | 5.05 | 2.99E−06 | 1.62 | ICMT | 3.44 | 2.45E−03 | 0.6 | RBX1 | 2.44 | 4.54E−02 | −0.02 |
| EHD1 | 8.91 | 9.39E−18 | 0.75 | CUL7 | 5.05 | 2.94E−06 | 0.6 | TRAPPC2L | 3.44 | 2.46E−03 | 0.53 | ABCA3 | 2.44 | 4.64E−02 | −0.02 |
| ZNF280D | 8.91 | 8.80E−18 | 0.48 | TBC1D10B | 5.05 | 2.98E−06 | 0.58 | CBR1 | 3.44 | 2.46E−03 | 0.47 | CUL9 | 2.44 | 4.55E−02 | −0.04 |
| HIST1H2BB | 8.9 | 1.03E−17 | 2.46 | GABARAPL1 | 5.05 | 3.03E−06 | 0.5 | DAGLA | 3.44 | 2.44E−03 | 0.42 | PSMD5 | 2.44 | 4.59E−02 | −0.08 |
| CACNA2D1 | 8.9 | 9.95E−18 | 0.88 | LRRC14 | 5.05 | 2.97E−06 | 0.41 | PINK1 | 3.44 | 2.48E−03 | 0.32 | NUDT2 | 2.44 | 4.55E−02 | −0.09 |
| THSD7A | 8.9 | 9.80E−18 | 0.65 | SERPINB5 | 5.05 | 2.99E−06 | 0.41 | DCAF15 | 3.44 | 2.46E−03 | 0.23 | RXRB | 2.44 | 4.55E−02 | −0.11 |
| SNRPA | 8.9 | 9.80E−18 | 0.65 | EXOSC1 | 5.05 | 3.01E−06 | 0.33 | SURF6 | 3.44 | 2.49E−03 | 0.22 | AC159540.2 | 2.43 | 4.68E−02 | 3.05 |
| TBC1D17 | 8.89 | 1.08E−17 | 0.7 | IPPK | 5.05 | 2.91E−06 | 0.29 | TBC1D25 | 3.44 | 2.45E−03 | 0.15 | CUX2 | 2.13 | 4.74E−02 | 1.22 |
| LRRC69 | 8.89 | 1.05E−17 | 0.67 | LHPP | 5.05 | 2.92E−06 | 0.28 | RITA1 | 3.43 | 2.51E−03 | 1.56 | FAM184A | 2.43 | 4.76E−02 | 0.65 |
| TOM1 | 8.89 | 1.04E−17 | 0.57 | NCS1 | 5.04 | 3.19E−06 | 0.9 | NDUFA6 | 3.43 | 2.57E−03 | 0.77 | SPA17 | 2.43 | 4.65E−02 | 0.64 |
| NUPR1 | 8.86 | 1.44E−17 | 2.05 | C6orf48 | 5.04 | 3.10E−06 | 0.65 | MRPS33 | 3.43 | 2.52E−03 | 0.75 | LURAP1L-AS1 | 2.43 | 4.66E−02 | 0.21 |
| LRP1B | 8.86 | 1.35E−17 | 1.22 | RHOF | 5.04 | 3.05E−06 | 0.52 | MRPL11 | 3.43 | 2.56E−03 | 0.73 | RAPGEF4 | 2.43 | 4.73E−02 | 0.2 |
| CHCHD2 | 8.86 | 1.42E−17 | 0.77 | LAMTOR4 | 5.04 | 3.06E−06 | 0.43 | FBXL14 | 3.43 | 2.55E−03 | 0.48 | COMTD1 | 2.43 | 4.69E−02 | 0.1 |
| HSH2D | 8.86 | 1.46E−17 | 0.69 | SLC44A2 | 5.04 | 3.11E−06 | 0.31 | NSA2 | 3.43 | 2.56E−03 | 0.26 | B4GALNT3 | 2.43 | 4.75E−02 | 0.07 |
| LVRN | 8.85 | 1.57E−17 | 3.74 | DPP7 | 5.04 | 3.13E−06 | 0.3 | BBC3 | 3.43 | 2.58E−03 | 0.26 | SDAD1 | 2.43 | 4.70E−02 | −0.03 |
| IFT57 | 8.85 | 1.50E−17 | 0.79 | LAMC2 | 5.04 | 3.07E−06 | 0.24 | ULK2 | 3.43 | 2.52E−03 | 0.24 | ATAD3B | 2.43 | 4.68E−02 | −0.04 |
| MUC16 | 8.85 | 1.56E−17 | 0.76 | CHP1 | 5.04 | 3.09E−06 | 0.19 | MCRS1 | 3.43 | 2.56E−03 | 0.24 | LRRC6 | 2.43 | 4.66E−02 | −0.05 |
| HAGH | 8.85 | 1.49E−17 | 0.73 | AP002884.2 | 5.03 | 3.25E−06 | 3.11 | LYRM1 | 3.43 | 2.55E−03 | 0.22 | PRNCR1 | 2.42 | 4.84E−02 | 2.87 |
| GOLGA2 | 8.85 | 1.54E−17 | 0.45 | CHST12 | 5.03 | 3.21E−06 | 0.74 | WDYHV1 | 3.43 | 2.54E−03 | 0.2 | ZAN | 2.42 | 4.82E−02 | 1.82 |
| TCF25 | 8.85 | 1.53E−17 | 0.36 | SPATA33 | 5.03 | 3.25E−06 | 0.48 | PKP3 | 3.43 | 2.58E−03 | 0.17 | TTLL9 | 2.42 | 4.79E−02 | 1.14 |
| LINC01091 | 8.84 | 1.66E−17 | 0.77 | ARL8A | 5.03 | 3.34E−06 | 0.43 | NIPSNAP3A | 3.43 | 2.55E−03 | 0.16 | ETV7 | 2.42 | 4.86E−02 | 0.85 |
| CTSA | 8.83 | 1.89E−17 | 0.69 | ARFIP2 | 5.03 | 3.24E−06 | 0.38 | SYNJ2 | 3.43 | 2.55E−03 | 0.09 | PORCN | 2.42 | 4.79E−02 | 0.72 |
| MMP24 | 8.83 | 1.76E−17 | 0.67 | ANKRD13A | 5.03 | 3.29E−06 | 0.31 | RNF220 | 3.43 | 2.57E−03 | 0.08 | SPIN4 | 2.42 | 4.82E−02 | 0.66 |
| SH2B3 | 8.82 | 2.07E−17 | 1.12 | LRRC41 | 5.03 | 3.26E−06 | 0.29 | AMT | 3.42 | 2.67E−03 | 1.74 | C22orf23 | 2.42 | 4.83E−02 | 0.66 |
| HMGAI | 8.82 | 1.92E−17 | 0.81 | NCL | 5.03 | 3.22E−06 | 0.27 | ITGA11 | 3.42 | 2.60E−03 | 1.28 | DGUOK-AS1 | 2.42 | 4.88E−02 | 0.61 |
| RASAL1 | 8.81 | 2.15E−17 | 0.68 | RANGAP1 | 5.03 | 3.25E−06 | 0.24 | RSAD2 | 3.42 | 2.63E−03 | 1.01 | FAM198B | 2.42 | 4.77E−02 | 0.44 |
| FANK1 | 8.81 | 2.13E−17 | 0.51 | STAMBP | 5.03 | 3.31E−06 | 0.18 | ABCA2 | 3.42 | 2.67E−03 | 0.48 | ZNF513 | 2.42 | 4.78E−02 | 0.35 |
| RPS6 | 8.8 | 2.29E−17 | 0.54 | PLPP4 | 5.02 | 3.48E−06 | 1.13 | BRI3BP | 3.42 | 2.64E−03 | 0.25 | PLCL1 | 2.42 | 4.85E−02 | 0.11 |
| CCDC85B | 8.79 | 2.47E−17 | 1.58 | PHF1 | 5.02 | 3.36E−06 | 0.41 | AGAP5 | 3.42 | 2.63E−03 | 0.21 | ZFYVE21 | 2.42 | 4.80E−02 | 0.05 |
| SNRPG | 8.78 | 2.77E−17 | 0.92 | DFFA | 5.02 | 3.43E−06 | 0.41 | MPPE1 | 3.42 | 2.64E−03 | 0.13 | UTP20 | 2.42 | 4.79E−02 | 0 |
| IARS | 8.78 | 2.93E−17 | 0.46 | TCP11L1 | 5.02 | 3.46E−06 | 0.35 | IQCB1 | 3.42 | 2.68E−03 | 0.1 | PICK1 | 2.42 | 4.85E−02 | −0.03 |
| LINC01170 | 8.77 | 3.00E−17 | 2.4 | ZCCHC17 | 5.02 | 3.50E−06 | 0.27 | POMGNT1 | 3.42 | 2.63E−03 | 0.08 | EFTUD2 | 2.42 | 4.86E−02 | −0.03 |
| PRKD3 | 8.77 | 3.01E−17 | 0.58 | PKD1 | 5.02 | 3.40E−06 | 0.26 | COPS8 | 3.42 | 2.67E−03 | −0.03 | DUSP11 | 2.42 | 4.84E−02 | −0.04 |
| GOLIM4 | 8.77 | 3.18E−17 | 0.49 | WLS | 5.02 | 3.48E−06 | 0.24 | LINC00942 | 3.41 | 2.74E−03 | 1.33 | STXBP2 | 2.42 | 4.87E−02 | −0.07 |
| SNHG25 | 8.76 | 3.27E−17 | 0.56 | TSTA3 | 5.02 | 3.53E−06 | 0.18 | DENND2A | 3.41 | 2.73E−03 | 0.73 | FUOM | 2.41 | 4.94E−02 | 1.59 |
| SERP1 | 8.76 | 3.39E−17 | 0.44 | MEF2C-AS1 | 5.01 | 3.68E−06 | 1.05 | PRDX3 | 3.41 | 2.72E−03 | 0.46 | CCBE1 | 2.41 | 4.91E−02 | 1.1 |
| OLFM4 | 8.75 | 3.54E−17 | 1.11 | STARD3NL | 5.01 | 3.60E−06 | 0.43 | HSPBP1 | 3.41 | 2.74E−03 | 0.42 | TCN2 | 2.41 | 4.90E−02 | 0.82 |
| CDKNIA | 8.75 | 3.65E−17 | 0.87 | TMED4 | 5.01 | 3.68E−06 | 0.39 | UPP2 | 3.41 | 2.72E−03 | 0.3 | UNC119B | 2.41 | 4.88E−02 | 0.53 |
| FLYWCH1 | 8.75 | 3.60E−17 | 0.61 | RPL22 | 5.01 | 3.66E−06 | 0.35 | C6orf203 | 3.41 | 2.72E−03 | 0.25 | GPR135 | 2.41 | 4.97E−02 | 0.29 |
| GUK1 | 8.75 | 3.59E−17 | 0.45 | PIGL | 5.01 | 3.53E−06 | 0.23 | HECW2 | 3.41 | 2.70E−03 | 0.21 | ZNF579 | 2.41 | 4.98E−02 | 0.28 |
| HNRNPL | 8.74 | 3.88E−17 | 0.4 | TMCO1 | 5.01 | 3.55E−06 | 0.22 | MEF2A | 3.41 | 2.69E−03 | 0.14 | ADGRG2 | 2.41 | 4.96E−02 | 0.24 |
| MAP3K5 | 8.74 | 4.12E−17 | 0.36 | ERI3 | 5.01 | 3.61E−06 | 0.22 | MOB1A | 3.41 | 2.72E−03 | 0.14 | APEH | 2.41 | 4.90E−02 | 0.19 |
| GGT1 | 8.73 | 4.53E−17 | 1.81 | RNF10 | 5.01 | 3.58E−06 | 0.2 | ZMAT3 | 3.41 | 2.76E−03 | 0.14 | NOL7 | 2.41 | 4.91E−02 | 0.1 |
| RNF4 | 8.73 | 4.38E−17 | 0.45 | MYOM1 | 5.01 | 3.58E−06 | 0.17 | SPIN1 | 3.41 | 2.77E−03 | 0.14 | ZDHHC11 | 2.41 | 4.92E−02 | 0.04 |
| SLC35D2 | 8.72 | 4.85E−17 | 0.51 | SOD3 | 5 | 3.84E−06 | 2.8 | ZNF461 | 3.41 | 2.74E−03 | 0.12 | EIF4E | 2.41 | 4.95E−02 | 0 |
| RTCB | 8.71 | 5.33E−17 | 0.61 | UBOX5 | 5 | 3.76E−06 | 0.57 | CFAP100 | 3.4 | 2.87E−03 | 2.15 | NDUFB2 | 2.41 | 4.90E−02 | −0.03 |
| TEAD3 | 8.71 | 5.18E−17 | 0.57 | UBB | 5 | 3.86E−06 | 0.39 | HSF2BP | 3.4 | 2.79E−03 | 0.69 | YPEL5 | 2.41 | 4.93E−02 | −0.08 |
| LYPD6B | 8.71 | 5.16E−17 | 0.56 | XBP1 | 5 | 3.89E−06 | 0.22 | PKD2L2 | 3.4 | 2.81E−03 | 0.68 | EFCAB8 | 2.4 | 5.10E−02 | 1.41 |
| PER1 | 8.71 | 5.18E−17 | 0.54 | PIPOX | 4.99 | 4.01E−06 | 1.13 | NTAN1 | 3.4 | 2.87E−03 | 0.38 | ADAMTSL3 | 2.4 | 5.06E−02 | 0.83 |
| TDG | 8.7 | 5.85E−17 | 0.66 | SVIP | 4.99 | 3.96E−06 | 0.57 | YIPF5 | 3.4 | 2.79E−03 | 0.31 | CFAP77 | 2.4 | 5.02E−02 | 0.61 |
| RASSF7 | 8.7 | 5.47E−17 | 0.48 | CARD19 | 4.99 | 3.98E−06 | 0.33 | RRAS | 3.4 | 2.83E−03 | 0.25 | CCDC73 | 2.4 | 5.02E−02 | 0.6 |
| EHD4 | 8.69 | 5.92E−17 | 0.59 | TELO2 | 4.99 | 3.91E−06 | 0.3 | MNT | 3.4 | 2.82E−03 | 0.13 | ABHD10 | 2.4 | 5.09E−02 | 0.39 |
| DYNLL2 | 8.68 | 6.77E−17 | 1.12 | SPG21 | 4.99 | 3.97E−06 | 0.29 | DENND2D | 3.4 | 2.85E−03 | 0.12 | SLC2A6 | 2.4 | 5.07E−02 | 0.3 |
| EIF2AK2 | 8.68 | 6.44E−17 | 0.45 | SLC12A8 | 4.99 | 4.06E−06 | 0.28 | SLC2A4RG | 3.4 | 2.78E−03 | 0.11 | AK7 | 2.4 | 5.11E−02 | 0.27 |
| BTBD9 | 8.68 | 6.39E−17 | 0.34 | IGF2 | 4.98 | 4.10E−06 | 7.66 | NT5C3A | 3.4 | 2.84E−03 | 0.11 | NDUFS7 | 2.4 | 5.11E−02 | 0.17 |
| SLFNL1 | 8.67 | 7.09E−17 | 1.31 | NINJ1 | 4.98 | 4.11E−06 | 1.53 | RPL15 | 3.4 | 2.86E−03 | 0.08 | SNX11 | 2.4 | 5.02E−02 | 0.15 |
| LEKR1 | 8.67 | 7.31E−17 | 1 | ZNF850 | 4.98 | 4.19E−06 | 0.81 | FAM162A | 3.4 | 2.82E−03 | 0.06 | GMIP | 2.4 | 5.06E−02 | 0.11 |
| COPB2 | 8.66 | 7.78E−17 | 0.47 | ZDHHC1 | 4.98 | 4.23E−06 | 0.59 | SLC5A11 | 3.39 | 2.88E−03 | 0.99 | TAPBPL | 2.4 | 5.02E−02 | 0.08 |
| TNC | 8.65 | 8.76E−17 | 1.56 | COIL | 4.98 | 4.11E−06 | 0.4 | FADD | 3.39 | 2.91E−03 | 0.82 | TAB2 | 2.4 | 5.12E−02 | 0.07 |
| SSBP3 | 8.65 | 8.95E−17 | 0.47 | ADAM19 | 4.98 | 4.20E−06 | 0.37 | RAB32 | 3.39 | 2.88E−03 | 0.64 | KRTAP5-AS1 | 2.4 | 5.11E−02 | 0.06 |
| TM4SF1-AS1 | 8.64 | 9.53E−17 | 1.35 | UBE2J1 | 4.98 | 4.10E−06 | 0.31 | SUPT4H1 | 3.39 | 2.96E−03 | 0.47 | OCIAD2 | 2.4 | 5.08E−02 | 0.01 |
| BCO2 | 8.64 | 9.60E−17 | 1.19 | ARL16 | 4.98 | 4.11E−06 | 0.28 | PNO1 | 3.39 | 2.97E−03 | 0.42 | CORO1B | 2.4 | 5.11E−02 | −0.01 |
| EMC10 | 8.64 | 9.55E−17 | 0.81 | S100A6 | 4.98 | 4.10E−06 | 0.18 | TFIP11 | 3.39 | 2.93E−03 | 0.4 | KNTC1 | 2.4 | 5.12E−02 | −0.01 |
| TNS1 | 8.64 | 9.50E−17 | 0.64 | ORAI3 | 4.97 | 4.32E−06 | 1.1 | ST3GAL4 | 3.39 | 2.95E−03 | 0.34 | EDEM2 | 2.4 | 5.03E−02 | −0.02 |
| RSRC2 | 8.64 | 9.26E−17 | 0.46 | JOSD1 | 4.97 | 4.32E−06 | 0.25 | ERBB4 | 3.39 | 2.88E−03 | 0.33 | TRMU | 2.4 | 5.11E−02 | −0.02 |
| SLC13A3 | 8.63 | 1.05E−16 | 1.11 | TMEM59 | 4.97 | 4.36E−06 | 0.23 | ISCA1 | 3.39 | 2.90E−03 | 0.32 | CTDSP1 | 2.4 | 5.02E−02 | −0.04 |
| NEK11 | 8.63 | 9.80E−17 | 0.46 | PTPN11 | 4.97 | 4.50E−06 | 0.23 | FAM228B | 3.39 | 2.89E−03 | 0.27 | MAPKAPK5 | 2.4 | 5.08E−02 | −0.05 |
| GRINA | 8.62 | 1.15E−16 | 0.84 | EML2 | 4.97 | 4.31E−06 | 0.21 | GJB3 | 3.39 | 2.93E−03 | 0.27 | PIGB | 2.4 | 5.04E−02 | −0.06 |
| PPP1R2 | 8.6 | 1.29E−16 | 0.45 | DDX24 | 4.97 | 4.33E−06 | 0.21 | TYW3 | 3.39 | 2.97E−03 | 0.24 | USP44 | 2.39 | 5.16E−02 | 1.69 |
| KIF12 | 8.58 | 1.53E−16 | 1.3 | MYO9B | 4.97 | 4.40E−06 | 0.2 | CCDC112 | 3.39 | 2.96E−03 | 0.22 | PAX6 | 2.39 | 5.22E−02 | 0.9 |
| CLIC6 | 8.58 | 1.50E−16 | 1.25 | RABL3 | 4.96 | 4.55E−06 | 0.36 | LINC01504 | 3.39 | 2.93E−03 | 0.13 | ORAI1 | 2.39 | 5.19E−02 | 0.86 |
| DST | 8.58 | 1.62E−16 | 0.24 | MITF | 4.96 | 4.72E−06 | 0.25 | MBNL1 | 3.39 | 2.94E−03 | 0.08 | NCDN | 2.39 | 5.24E−02 | 0.69 |
| GTF2IRD2B | 8.57 | 1.74E−16 | 0.65 | PRSS21 | 4.95 | 4.86E−06 | 1.14 | ZNF567 | 3.39 | 2.95E−03 | 0.03 | KRT86 | 2.39 | 5.13E−02 | 0.45 |
| ABHD11 | 8.57 | 1.64E−16 | 0.64 | CTTNBP2 | 4.95 | 4.92E−06 | 1.12 | MGAT4C | 3.38 | 2.97E−03 | 1.09 | EGF | 2.39 | 5.24E−02 | 0.38 |
| BLVRB | 8.57 | 1.72E−16 | 0.58 | PTPRT | 4.95 | 4.97E−06 | 1.12 | MAPK12 | 3.38 | 3.01E−03 | 0.98 | ZNF528 | 2.39 | 5.24E−02 | 0.22 |
| ADM | 8.55 | 2.01E−16 | 1.19 | TMEM179B | 4.95 | 4.87E−06 | 0.42 | FAM84A | 3.38 | 2.99E−03 | 0.83 | DHRS4L2 | 2.39 | 5.24E−02 | 0.2 |
| PLA2G12A | 8.55 | 1.96E−16 | 1.01 | HPS3 | 4.95 | 4.79E−06 | 0.27 | CALN1 | 3.38 | 2.97E−03 | 0.37 | ERC2 | 2.39 | 5.23E−02 | 0.16 |
| RPL36AL | 8.55 | 2.07E−16 | 0.75 | LATS2 | 4.95 | 4.95E−06 | 0.27 | NIFK | 3.38 | 2.97E−03 | 0.17 | ARRDC3-AS1 | 2.39 | 5.20E−02 | 0.09 |
| APOL2 | 8.55 | 2.00E−16 | 0.51 | KMT2D | 4.95 | 4.74E−06 | 0.23 | CBY1 | 3.38 | 3.05E−03 | 0.17 | HK1 | 2.39 | 5.13E−02 | 0.07 |
| GDI1 | 8.54 | 2.28E−16 | 0.65 | HECTD4 | 4.95 | 4.77E−06 | 0.22 | CISD1 | 3.38 | 3.05E−03 | 0.13 | ICE1 | 2.39 | 5.23E−02 | 0.04 |
| PPM1L | 8.53 | 2.41E−16 | 0.66 | CDC42EP4 | 4.95 | 4.95E−06 | 0.15 | MDH1 | 3.38 | 3.05E−03 | 0.11 | MRPL48 | 2.39 | 5.23E−02 | 0.03 |
| HSF1 | 8.53 | 2.28E−16 | 0.44 | HLA-DQB1 | 4.94 | 5.12E−06 | 1.66 | CAAP1 | 3.38 | 2.99E−03 | 0.06 | DHX38 | 2.39 | 5.21E−02 | 0.01 |
| LSM4 | 8.52 | 2.70E−16 | 0.9 | KDSR | 4.94 | 5.11E−06 | 0.38 | MRPS35 | 3.38 | 3.00E−03 | 0.06 | COA5 | 2.39 | 5.22E−02 | 0 |
| MFSD11 | 8.52 | 2.61E−16 | 0.46 | PITRM1 | 4.94 | 5.00E−06 | 0.28 | SSR2 | 3.38 | 3.03E−03 | 0.04 | PLEKHM3 | 2.39 | 5.14E−02 | −0.03 |
| ZDHHC14 | 8.52 | 2.57E−16 | 0.42 | ARHGAP44 | 4.94 | 5.03E−06 | 0.28 | FOSL2 | 3.38 | 2.97E−03 | 0.03 | ZNF823 | 2.39 | 5.19E−02 | −0.03 |
| HSD17B3 | 8.51 | 2.77E−16 | 4.56 | LRRK1 | 4.94 | 5.04E−06 | 0.27 | POLR2I | 3.37 | 3.13E−03 | 1.7 | VPS26B | 2.39 | 5.14E−02 | −0.06 |
| DENND3 | 8.51 | 2.72E−16 | 0.57 | UNC13D | 4.94 | 5.01E−06 | 0.2 | RPP25 | 3.37 | 3.11E−03 | 1.53 | CNBD1 | 2.38 | 5.28E−02 | 0.8 |
| CNN2 | 8.5 | 2.98E−16 | 0.63 | CNBP | 4.94 | 5.00E−06 | 0.18 | HERC5 | 3.37 | 3.17E−03 | 1.23 | CRYM | 2.38 | 5.27E−02 | 0.72 |
| LDHA | 8.5 | 3.00E−16 | 0.5 | CLN5 | 4.93 | 5.40E−06 | 2.63 | BUD31 | 3.37 | 3.17E−03 | 0.44 | MXRA8 | 2.38 | 5.33E−02 | 0.46 |
| OAS2 | 8.49 | 3.29E−16 | 2.25 | MRPL36 | 4.93 | 5.43E−06 | 1.69 | USP11 | 3.37 | 3.10E−03 | 0.41 | RDH14 | 2.38 | 5.27E−02 | 0.36 |
| SP100 | 8.49 | 3.33E−16 | 0.37 | PCBD1 | 4.93 | 5.29E−06 | 0.88 | LINC00672 | 3.37 | 3.12E−03 | 0.4 | NME4 | 2.38 | 5.28E−02 | 0.3 |
| NDUFB10 | 8.48 | 3.77E−16 | 1.36 | SSR1 | 4.93 | 5.47E−06 | 0.32 | IFNLR1 | 3.37 | 3.13E−03 | 0.38 | LOXL2 | 2.38 | 5.30E−02 | 0.27 |
| TBC1D22B | 8.48 | 3.76E−16 | 0.51 | ZNF267 | 4.93 | 5.43E−06 | 0.24 | MRPL2 | 3.37 | 3.09E−03 | 0.35 | TMEM72-AS1 | 2.38 | 5.36E−02 | 0.22 |
| IFT140 | 8.48 | 3.76E−16 | 0.51 | TXLNG | 4.93 | 5.43E−06 | 0.23 | SSRP1 | 3.37 | 3.15E−03 | 0.18 | RCCD1 | 2.38 | 5.38E−02 | 0.11 |
| TPM1 | 8.47 | 4.01E−16 | 0.38 | MTCH2 | 4.93 | 5.21E−06 | 0.22 | GAS6-AS1 | 3.37 | 3.15E−03 | 0.16 | TMLHE | 2.38 | 5.33E−02 | 0.1 |
| SP140L | 8.46 | 4.22E−16 | 0.51 | ZNF790 | 4.93 | 5.26E−06 | 0.2 | DDX56 | 3.37 | 3.16E−03 | 0.11 | KLHL21 | 2.38 | 5.37E−02 | 0.03 |
| TMEM184B | 8.44 | 5.12E−16 | 0.44 | GCLC | 4.93 | 5.26E−06 | 0.2 | EHBP1 | 3.37 | 3.16E−03 | 0.08 | YBX1 | 2.38 | 5.26E−02 | −0.11 |
| PFN1 | 8.43 | 5.45E−16 | 0.7 | SLFN5 | 4.93 | 5.39E−06 | 0.19 | ATN1 | 3.37 | 3.15E−03 | 0.04 | ZNF627 | 2.38 | 5.32E−02 | −0.11 |
| AHNAK | 8.43 | 5.39E−16 | 0.34 | TRMT61B | 4.92 | 5.71E−06 | 0.24 | SUN2 | 3.37 | 3.17E−03 | 0.03 | PRX | 2.37 | 5.48E−02 | 1.5 |
| KIAA1211L | 8.42 | 6.04E−16 | 0.47 | NOTCH2 | 4.92 | 5.64E−06 | 0.23 | DCXR | 3.36 | 3.18E−03 | 0.89 | SERTAD4-AS1 | 2.37 | 5.44E−02 | 1.11 |
| TBCE | 8.41 | 6.76E−16 | 1.72 | RPL7L1 | 4.92 | 5.60E−06 | 0.22 | SNHG21 | 3.36 | 3.24E−03 | 0.77 | CCDC113 | 2.37 | 5.38E−02 | 0.64 |
| TPST1 | 8.41 | 6.52E−16 | 0.38 | ADPGK | 4.92 | 5.60E−06 | 0.21 | PLCXD2 | 3.36 | 3.22E−03 | 0.62 | CTU1 | 2.37 | 5.50E−02 | 0.52 |
| VASP | 8.41 | 6.35E−16 | 0.37 | CLUH | 4.92 | 5.69E−06 | 0.21 | ERICH6-AS1 | 3.36 | 3.19E−03 | 0.57 | MRPL44 | 2.37 | 5.41E−02 | 0.41 |
| SDC4 | 8.4 | 7.28E−16 | 0.54 | XRCC6 | 4.92 | 5.71E−06 | 0.19 | C19orf48 | 3.36 | 3.18E−03 | 0.37 | TRAF1 | 2.37 | 5.49E−02 | 0.38 |
| LPINI | 8.4 | 7.23E−16 | 0.46 | PLEKHM1 | 4.92 | 5.66E−06 | 0.17 | TET3 | 3.36 | 3.24E−03 | 0.13 | OSCP1 | 2.37 | 5.42E−02 | 0.36 |
| WNT5B | 8.38 | 8.10E−16 | 0.75 | PSME2 | 4.91 | 5.91E−06 | 0.84 | POLR2E | 3.36 | 3.27E−03 | 0.12 | BCAS2 | 2.37 | 5.44E−02 | 0.08 |
| VCAN | 8.38 | 8.22E−16 | 0.58 | ACTR1A | 4.91 | 5.95E−06 | 0.4 | SLC4A2 | 3.36 | 3.24E−03 | 0.11 | C19orf25 | 2.37 | 5.49E−02 | −0.02 |
| ESF1 | 8.37 | 8.83E−16 | 0.45 | OSGIN2 | 4.91 | 5.74E−06 | 0.29 | UNK | 3.36 | 3.18E−03 | 0.09 | ZNF571 | 2.37 | 5.48E−02 | −0.03 |
| ACHE | 8.36 | 9.68E−16 | 1.36 | C6orf62 | 4.91 | 5.91E−06 | 0.21 | TMEM134 | 3.36 | 3.18E−03 | 0.06 | CFAP97 | 2.37 | 5.41E−02 | −0.05 |
| NUCB1 | 8.36 | 9.64E−16 | 0.74 | MCCC1 | 4.91 | 5.83E−06 | 0.2 | CALCOCO1 | 3.36 | 3.19E−03 | 0.05 | NOLC1 | 2.37 | 5.43E−02 | −0.1 |
| PKN1 | 8.36 | 9.64E−16 | 0.5 | SPR | 4.9 | 6.28E−06 | 1.78 | P2RX7 | 3.35 | 3.30E−03 | 1.6 | POLH | 2.37 | 5.38E−02 | −0.11 |
| TSPAN1 | 8.36 | 1.02E−15 | 0.46 | DYRK1B | 4.9 | 6.21E−06 | 0.81 | DNAH9 | 3.35 | 3.28E−03 | 0.92 | AC007091.1 | 2.36 | 5.63E−02 | 3.06 |
| WARS | 8.35 | 1.10E−15 | 0.71 | YWHAH | 4.9 | 6.19E−06 | 0.53 | LINC01470 | 3.35 | 3.37E−03 | 0.91 | AC010976.2 | 2.36 | 5.53E−02 | 1.75 |
| FRMD8 | 8.35 | 1.06E−15 | 0.54 | PSMA4 | 4.9 | 6.07E−06 | 0.34 | THOC6 | 3.35 | 3.31E−03 | 0.63 | SSC5D | 2.36 | 5.54E−02 | 1.13 |
| COG1 | 8.34 | 1.14E−15 | 0.82 | PISD | 4.9 | 6.06E−06 | 0.17 | ILIRN | 3.35 | 3.37E−03 | 0.29 | RPL26L1 | 2.36 | 5.57E−02 | 0.69 |
| NFIA | 8.34 | 1.14E−15 | 0.33 | TMEM39A | 4.9 | 6.03E−06 | 0.15 | TRUB2 | 3.35 | 3.28E−03 | 0.27 | MN1 | 2.36 | 5.52E−02 | 0.61 |
| CHST3 | 8.33 | 1.23E−15 | 1.3 | KLHL5 | 4.9 | 6.23E−06 | 0.15 | AP1S2 | 3.35 | 3.36E−03 | 0.26 | TMEM186 | 2.36 | 5.59E−02 | 0.6 |
| MPHOSPH9 | 8.33 | 1.28E−15 | 0.63 | ORMDL2 | 4.89 | 6.49E−06 | 1.22 | TFF2 | 3.35 | 3.33E−03 | 0.25 | ZNF606 | 2.36 | 5.59E−02 | 0.48 |
| CLEC2D | 8.33 | 1.28E−15 | 0.6 | SLC38A5 | 4.89 | 6.43E−06 | 1.01 | OXA1L | 3.35 | 3.32E−03 | 0.23 | AC016735.1 | 2.36 | 5.60E−02 | 0.48 |
| FAM227A | 8.32 | 1.38E−15 | 0.91 | TSSC4 | 4.89 | 6.58E−06 | 0.82 | CREB3 | 3.35 | 3.28E−03 | 0.2 | ANKEF1 | 2.36 | 5.60E−02 | 0.39 |
| ARPC5L | 8.32 | 1.32E−15 | 0.87 | PARVB | 4.89 | 6.39E−06 | 0.7 | SIKE1 | 3.35 | 3.30E−03 | 0.19 | FITM2 | 2.36 | 5.56E−02 | 0.31 |
| SSR3 | 8.32 | 1.42E−15 | 0.6 | BCORL1 | 4.89 | 6.5OE−06 | 0.53 | MAN1B1 | 3.35 | 3.28E−03 | 0.12 | ADHFE1 | 2.36 | 5.58E−02 | 0.26 |
| PTMA | 8.32 | 1.41E−15 | 0.43 | ZNF785 | 4.89 | 6.41E−06 | 0.39 | COL5A2 | 3.35 | 3.33E−03 | 0.09 | ANXA13 | 2.36 | 5.64E−02 | 0.18 |
| TRERF1 | 8.31 | 1.54E−15 | 0.37 | CRIM1 | 4.89 | 6.38E−06 | 0.24 | KIF3A | 3.35 | 3.32E−03 | 0.07 | CAPN1 | 2.36 | 5.52E−02 | 0.07 |
| ERP29 | 8.3 | 1.67E−15 | 0.97 | SFI1 | 4.89 | 6.42E−06 | 0.17 | DCAF16 | 3.35 | 3.33E−03 | 0.01 | SFTA2 | 2.36 | 5.52E−02 | 0.05 |
| PITPNA | 8.3 | 1.57E−15 | 0.44 | SLPI | 4.88 | 6.84E−06 | 0.56 | AP3S2 | 3.34 | 3.47E−03 | 0.98 | DAGLB | 2.36 | 5.55E−02 | −0.03 |
| FAM102A | 8.3 | 1.57E−15 | 0.39 | MUC3A | 4.88 | 6.64E−06 | 0.51 | CLCN4 | 3.34 | 3.48E−03 | 0.8 | SSBP4 | 2.36 | 5.63E−02 | −0.03 |
| 9-Sep | 8.3 | 1.67E−15 | 0.25 | POLA2 | 4.88 | 6.85E−06 | 0.38 | ZDHHC11B | 3.34 | 3.40E−03 | 0.7 | CCNC | 2.36 | 5.64E−02 | −0.03 |
| CD81 | 8.29 | 1.81E−15 | 1.01 | LACTB | 4.88 | 6.90E−06 | 0.38 | CDK4 | 3.34 | 3.41E−03 | 0.48 | NECAP1 | 2.36 | 5.53E−02 | −0.04 |
| C9orf3 | 8.29 | 1.75E−15 | 0.25 | TSN | 4.88 | 6.71E−06 | 0.29 | SLFN12 | 3.34 | 3.50E−03 | 0.3 | CBWD2 | 2.36 | 5.62E−02 | −0.04 |
| CHMP2A | 8.28 | 1.89E−15 | 1.1 | RARG | 4.88 | 6.72E−06 | 0.26 | MDFI | 3.34 | 3.44E−03 | 0.29 | CCT8 | 2.36 | 5.57E−02 | −0.08 |
| DRAM1 | 8.28 | 1.84E−15 | 0.89 | TIMM23 | 4.88 | 6.66E−06 | 0.22 | ZNF362 | 3.34 | 3.49E−03 | 0.28 | RAB30-AS1 | 2.36 | 5.64E−02 | −0.1 |
| PHLDB1 | 8.28 | 1.83E−15 | 0.57 | KIAA0556 | 4.88 | 6.71E−06 | 0.21 | CALM1 | 3.34 | 3.43E−03 | 0.15 | BEST4 | 2.35 | 5.75E−02 | 1.87 |
| ABCB9 | 8.27 | 2.05E−15 | 1.08 | TRAF3IP1 | 4.88 | 6.85E−06 | 0.21 | MAPRE1 | 3.34 | 3.46E−03 | 0.12 | LINC00954 | 2.35 | 5.75E−02 | 1.86 |
| PRKAR1B | 8.27 | 2.10E−15 | 0.63 | RAB12 | 4.88 | 6.84E−06 | 0.2 | SMARCD1 | 3.34 | 3.41E−03 | 0.11 | KCNE1 | 2.35 | 5.67E−02 | 1.67 |
| SCAND1 | 8.26 | 2.32E−15 | 1.07 | VPS29 | 4.38 | 6.74E−06 | 0.19 | DAZAP2 | 3.34 | 3.44E−03 | 0.11 | AC062017.1 | 2.35 | 5.68E−02 | 0.94 |
| ARHGAP22 | 8.26 | 2.22E−15 | 0.82 | SWI5 | 4.87 | 7.06E−06 | 0.94 | ERCC3 | 3.34 | 3.45E−03 | 0.04 | TOX2 | 2.35 | 5.65E−02 | 0.74 |
| CHMPIA | 8.26 | 2.28E−15 | 0.55 | PFDN6 | 4.87 | 7.03E−06 | 0.91 | TMC2 | 3.33 | 3.61E−03 | 1.69 | EXOSC5 | 2.35 | 5.78E−02 | 0.67 |
| BFAR | 8.26 | 2.33E−15 | 0.53 | SNX17 | 4.87 | 7.23E−06 | 0.91 | C12orf43 | 3.33 | 3.61E−03 | 0.73 | ATRNL1 | 2.35 | 5.67E−02 | 0.66 |
| CNTN4 | 8.25 | 2.39E−15 | 1.24 | PSME1 | 4.87 | 7.22E−06 | 0.63 | RBP5 | 3.33 | 3.57E−03 | 0.7 | LCA5L | 2.35 | 5.73E−02 | 0.57 |
| HCFC1R1 | 8.25 | 2.34E−15 | 1.2 | TEX2 | 4.87 | 7.14E−06 | 0.3 | SLC35A4 | 3.33 | 3.53E−03 | 0.64 | TIMM22 | 2.35 | 5.77E−02 | 0.55 |
| ACVR1B | 8.25 | 2.43E−15 | 0.43 | GTF3C1 | 4.87 | 7.18E−06 | 0.18 | DPAGT1 | 3.33 | 3.57E−03 | 0.62 | CKS1B | 2.35 | 5.67E−02 | 0.46 |
| DNMT3A | 8.24 | 2.64E−15 | 0.47 | TINAGL1 | 4.87 | 7.27E−06 | 0.18 | THAP7 | 3.33 | 3.59E−03 | 0.61 | KDF1 | 2.35 | 5.67E−02 | 0.27 |
| ZBTB38 | 8.24 | 2.67E−15 | 0.4 | AXIN1 | 4.87 | 7.21E−06 | 0.16 | YBEY | 3.33 | 3.51E−03 | 0.38 | SMIM2-AS1 | 2.35 | 5.74E−02 | 0.17 |
| NDUFB7 | 8.23 | 2.85E−15 | 1.02 | IGSF5 | 4.86 | 7.38E−06 | 1.51 | TPRN | 3.33 | 3.60E−03 | 0.37 | LIX1L | 2.35 | 5.68E−02 | 0.14 |
| YWHAB | 8.23 | 2.97E−15 | 0.52 | PEA15 | 4.86 | 7.53E−06 | 0.81 | CACNB3 | 3.33 | 3.52E−03 | 0.32 | SIK2 | 2.35 | 5.72E−02 | 0.08 |
| SNED1 | 8.23 | 2.78E−15 | 0.49 | LFNG | 4.86 | 7.50E−06 | 0.52 | MMD | 3.33 | 3.56E−03 | 0.3 | ZPR1 | 2.35 | 5.66E−02 | 0.07 |
| TCEAL4 | 8.22 | 3.01E−15 | 0.91 | TPST2 | 4.86 | 7.54E−06 | 0.48 | STOML2 | 3.33 | 3.58E−03 | 0.3 | CD99L2 | 2.35 | 5.65E−02 | 0.04 |
| BSDC1 | 8.22 | 3.22E−15 | 0.55 | BTN3A2 | 4.86 | 7.36E−06 | 0.46 | MKS1 | 3.33 | 3.60E−03 | 0.3 | GRB10 | 2.35 | 5.75E−02 | 0.03 |
| ARMC8 | 8.22 | 3.06E−15 | 0.39 | SLC22A18 | 4.86 | 7.36E−06 | 0.27 | TRAPPC2 | 3.33 | 3.52E−03 | 0.26 | METTL21A | 2.35 | 5.67E−02 | 0.02 |
| SLC26A8 | 8.21 | 3.34E−15 | 1.19 | HOMER1 | 4.86 | 7.53E−06 | 0.27 | SMIM4 | 3.33 | 3.58E−03 | 0.23 | SPINT2 | 2.35 | 5.75E−02 | 0 |
| EEPD1 | 8.21 | 3.28E−15 | 0.36 | SOX9 | 4.86 | 7.46E−06 | 0.26 | DNM3 | 3.33 | 3.54E−03 | 0.21 | GIPC2 | 2.35 | 5.72E−02 | −0.05 |
| RNF19B | 8.19 | 3.96E−15 | 0.6 | JTB | 4.86 | 7.32E−06 | 0.23 | ZMIZ2 | 3.33 | 3.51E−03 | 0.13 | ARFGAP2 | 2.35 | 5.65E−02 | −0.08 |
| SLC26A3 | 8.19 | 3.81E−15 | 0.51 | NR6A1 | 4.86 | 7.43E−06 | 0.2 | RALBP1 | 3.33 | 3.53E−03 | 0.13 | IDUA | 2.35 | 5.68E−02 | −0.11 |
| PRUNE2 | 8.17 | 4.49E−15 | 1.52 | ADAM8 | 4.85 | 7.96E−06 | 0.52 | MSI2 | 3.33 | 3.56E−03 | 0.11 | PDZD11 | 2.34 | 5.81E−02 | 0.66 |
| TMA7 | 8.17 | 4.71E−15 | 0.8 | WFDC3 | 4.85 | 7.66E−06 | 0.46 | CWF19L2 | 3.33 | 3.55E−03 | 0.09 | TFAP2E | 2.34 | 5.90E−02 | 0.61 |
| AP1B1 | 8.17 | 4.59E−15 | 0.65 | ATP6V0A2 | 4.85 | 7.97E−06 | 0.35 | TPD52L2 | 3.33 | 3.61E−03 | 0.09 | TUBG1 | 2.34 | 5.83E−02 | 0.55 |
| SLC25A6 | 8.16 | 4.87E−15 | 0.91 | TGFB2 | 4.85 | 7.95E−06 | 0.34 | UBA5 | 3.33 | 3.53E−03 | 0.05 | CCDC173 | 2.34 | 5.80E−02 | 0.53 |
| KRT10 | 8.16 | 4.88E−15 | 0.85 | ARF6 | 4.85 | 7.72E−06 | 0.25 | EIF4EBP2 | 3.33 | 3.57E−03 | 0.04 | RXFP1 | 2.34 | 5.86E−02 | 0.51 |
| LRRC37A2 | 8.16 | 5.18E−15 | 0.59 | RCC1 | 4.85 | 7.66E−06 | 0.23 | SLC14A2 | 3.32 | 3.67E−03 | 1.83 | TBX6 | 2.34 | 5.85E−02 | 0.47 |
| DIRC2 | 8.16 | 5.18E−15 | 0.58 | UHRF1BP1 | 4.85 | 7.96E−06 | 0.22 | RAB37 | 3.32 | 3.69E−03 | 1.83 | PDGFB | 2.34 | 5.85E−02 | 0.4 |
| RPL37 | 8.16 | 5.08E−15 | 0.57 | EMC6 | 4.84 | 8.30E−06 | 1.58 | DNAJC30 | 3.32 | 3.73E−03 | 1.06 | FBXO17 | 2.34 | 5.81E−02 | 0.37 |
| COL4A1 | 8.15 | 5.25E−15 | 1.03 | TMEM147 | 4.84 | 8.20E−06 | 0.79 | SLC7A8 | 3.32 | 3.69E−03 | 0.91 | MCAT | 2.34 | 5.88E−02 | 0.36 |
| IFI35 | 8.14 | 5.71E−15 | 1.31 | MAP3K6 | 4.84 | 8.23E−06 | 0.41 | TMEM86B | 3.32 | 3.73E−03 | 0.7 | KCTD16 | 2.34 | 5.79E−02 | 0.34 |
| MAP3K11 | 8.14 | 5.96E−15 | 0.77 | CCDC14 | 4.84 | 8.31E−06 | 0.22 | PCTP | 3.32 | 3.71E−03 | 0.47 | AKIP1 | 2.34 | 5.87E−02 | 0.32 |
| CLIC5 | 8.14 | 6.08E−15 | 0.77 | JUP | 4.84 | 8.31E−06 | 0.21 | TWISTNB | 3.32 | 3.73E−03 | 0.45 | DDX19A | 2.34 | 5.78E−02 | 0.23 |
| CYB561 | 8.14 | 6.14E−15 | 0.65 | PLEKHM2 | 4.84 | 8.13E−06 | 0.2 | DDX49 | 3.32 | 3.72E−03 | 0.26 | MRPS22 | 2.34 | 5.83E−02 | 0.15 |
| PHLDA2 | 8.14 | 5.77E−15 | 0.62 | CPT1A | 4.84 | 8.29E−06 | 0.2 | NDUFB1 | 3.32 | 3.62E−03 | 0.22 | SLC35A1 | 2.34 | 5.85E−02 | 0.14 |
| BANK1 | 8.14 | 5.83E−15 | 0.45 | PDGFC | 4.84 | 8.11E−06 | 0.18 | CD2BP2 | 3.32 | 3.62E−03 | 0.18 | GIT1 | 2.34 | 5.87E−02 | 0.12 |
| KDM4A | 8.13 | 6.44E−15 | 3.66 | DDX60 | 4.84 | 8.01E−06 | 0.17 | PXDC1 | 3.32 | 3.71E−03 | 0.16 | DOCK9 | 2.34 | 5.86E−02 | 0.11 |
| NDUFS5 | 8.13 | 6.40E−15 | 0.98 | YWHAQ | 4.84 | 8.27E−06 | 0.17 | PLEKHG2 | 3.32 | 3.68E−03 | 0.15 | BAZ2A | 2.34 | 5.89E−02 | 0.04 |
| YIPF2 | 8.13 | 6.55E−15 | 0.98 | SERTAD3 | 4.83 | 8.76E−06 | 1.19 | DDAH1 | 3.32 | 3.69E−03 | 0.15 | SCO1 | 2.34 | 5.83E−02 | 0 |
| COX4I1 | 8.13 | 6.38E−15 | 0.59 | ZNF540 | 4.83 | 8.70E−06 | 0.84 | GPALPP1 | 3.32 | 3.73E−03 | 0.09 | PAPOLG | 2.34 | 5.89E−02 | −0.05 |
| SEC22A | 8.13 | 6.21E−15 | 0.39 | CYP27C1 | 4.83 | 8.45E−06 | 0.71 | EIF2AK1 | 3.32 | 3.67E−03 | 0.07 | GNL2 | 2.34 | 5.78E−02 | −0.17 |
| ZNF428 | 8.12 | 6.89E−15 | 1.17 | IL1R1 | 4.83 | 8.57E−06 | 0.44 | RFWD3 | 3.32 | 3.67E−03 | 0.05 | KCNH7 | 2.33 | 5.95E−02 | 1.46 |
| NELFE | 8.12 | 7.14E−15 | 1.15 | TMEM185A | 4.83 | 8.76E−06 | 0.4 | LATS1 | 3.32 | 3.67E−03 | 0.03 | CPEB1 | 2.33 | 5.93E−02 | 1.42 |
| MZT2B | 8.12 | 6.97E−15 | 0.93 | SNRNP40 | 4.83 | 8.65E−06 | 0.29 | IFT22 | 3.31 | 3.76E−03 | 1.22 | LINC01099 | 2.33 | 5.95E−02 | 1.34 |
| COPB1 | 8.12 | 6.70E−15 | 0.4 | ARHGDIA | 4.83 | 8.57E−06 | 0.28 | EXOSC4 | 3.31 | 3.75E−03 | 1.12 | FDXR | 2.33 | 6.03E−02 | 0.54 |
| SLC51A | 8.11 | 7.34E−15 | 1.73 | STXBP4 | 4.83 | 8.42E−06 | 0.25 | UBL4A | 3.31 | 3.80E−03 | 0.89 | KMT2A | 2.33 | 6.04E−02 | 0.11 |
| ST14 | 8.1 | 7.89E−15 | 0.45 | MRPL3 | 4.83 | 8.61E−06 | 0.21 | C11orf68 | 3.31 | 3.83E−03 | 0.75 | PDE6D | 2.33 | 5.92E−02 | 0.07 |
| SKIL | 8.1 | 8.37E−15 | 0.33 | MFSD3 | 4.82 | 9.04E−06 | 1 | CHTF18 | 3.31 | 3.75E−03 | 0.64 | GAA | 2.33 | 5.92E−02 | 0.06 |
| PIGR | 8.09 | 8.64E−15 | 1.56 | TMEM222 | 4.82 | 9.11E−06 | 0.68 | GPANK1 | 3.31 | 3.82E−03 | 0.56 | DNAJB14 | 2.33 | 6.00E−02 | 0.06 |
| FBXO36 | 8.09 | 9.08E−15 | 0.7 | SNUPN | 4.82 | 9.09E−06 | 0.47 | TTC31 | 3.31 | 3.82E−03 | 0.32 | TNFAIP3 | 2.33 | 5.95E−02 | 0.05 |
| DCAF7 | 8.09 | 8.93E−15 | 0.4 | ELMO2 | 4.82 | 9.16E−06 | 0.4 | CDK9 | 3.31 | 3.84E−03 | 0.18 | REXO4 | 2.33 | 6.04E−02 | 0 |
| STX5 | 8.08 | 9.64E−15 | 0.44 | GAS6 | 4.82 | 8.96E−06 | 0.33 | IL15 | 3.31 | 3.76E−03 | 0.16 | SASH1 | 2.33 | 5.92E−02 | −0.03 |
| MYH9 | 8.08 | 9.54E−15 | 0.37 | YARS2 | 4.82 | 9.09E−06 | 0.31 | OSBP2 | 3.31 | 3.82E−03 | 0.12 | RNF44 | 2.33 | 5.96E−02 | −0.11 |
| HSBP1 | 8.07 | 1.04E−14 | 0.86 | TMEM50A | 4.82 | 9.13E−06 | 0.29 | VPS37C | 3.31 | 3.75E−03 | 0.1 | PLK3 | 2.33 | 5.97E−02 | −0.2 |
| CFAP54 | 8.06 | 1.16E−14 | 1.36 | GYS1 | 4.82 | 8.89E−06 | 0.28 | PGAP2 | 3.31 | 3.77E−03 | 0.09 | AC124861.1 | 2.32 | 6.12E−02 | 0.92 |
| CD99 | 8.06 | 1.16E−14 | 0.54 | RIC8B | 4.82 | 9.06E−06 | 0.23 | GPAA1 | 3.31 | 3.80E−03 | 0.09 | AC067956.1 | 2.32 | 6.12E−02 | 0.83 |
| LGALS4 | 8.06 | 1.12E−14 | 0.48 | LINC01317 | 4.81 | 9.57E−06 | 1.56 | CCS | 3.31 | 3.80E−03 | 0.09 | TBCC | 2.32 | 6.15E−02 | 0.54 |
| ARSG | 8.05 | 1.25E−14 | 0.79 | POLR2C | 4.81 | 9.61E−06 | 0.69 | MGAT4A | 3.31 | 3.76E−03 | 0.07 | ZNF622 | 2.32 | 6.20E−02 | 0.51 |
| RAD23A | 8.05 | 1.16E−14 | 0.52 | ARL4C | 4.81 | 9.52E−06 | 0.59 | MTHFR | 3.31 | 3.84E−03 | 0.01 | CNTN1 | 2.32 | 6.07E−02 | 0.47 |
| IFT172 | 8.04 | 1.30E−14 | 0.69 | OXLD1 | 4.81 | 9.65E−06 | 0.49 | TIMM17B | 3.3 | 3.97E−03 | 0.6 | EMC7 | 2.32 | 6.07E−02 | 0.27 |
| IRAK1 | 8.04 | 1.34E−14 | 0.61 | CRISPLD2 | 4.81 | 9.30E−06 | 0.46 | PPIC | 3.3 | 3.88E−03 | 0.45 | BTN3A3 | 2.32 | 6.10E−02 | 0.2 |
| SOX6 | 8.04 | 1.34E−14 | 0.45 | NSUN5 | 4.81 | 9.53E−06 | 0.29 | ZNF385D | 3.3 | 3.98E−03 | 0.36 | TRIQK | 2.32 | 6.14E−02 | 0 |
| LRRC37A3 | 8.03 | 1.37E−14 | 0.38 | KRTCAP3 | 4.81 | 9.48E−06 | 0.26 | TMEM143 | 3.3 | 3.93E−03 | 0.35 | MAT2B | 2.32 | 6.19E−02 | −0.01 |
| NEDD9 | 8.02 | 1.59E−14 | 0.43 | TPD52L1 | 4.81 | 9.29E−06 | 0.25 | ILVBL | 3.3 | 3.92E−03 | 0.27 | SH3GLB2 | 2.32 | 6.17E−02 | −0.04 |
| PTGES2 | 8.01 | 1.65E−14 | 0.55 | CABLES1 | 4.81 | 9.34E−06 | 0.2 | NT5C3B | 3.3 | 3.92E−03 | 0.22 | ZNF397 | 2.32 | 6.18E−02 | −0.04 |
| TMEM208 | 8 | 1.76E−14 | 1.06 | FAM3D | 4.8 | 9.68E−06 | 1.04 | SNX12 | 3.3 | 3.93E−03 | 0.21 | TMEM167A | 2.32 | 6.10E−02 | −0.07 |
| SLCO4A1 | 8 | 1.87E−14 | 0.94 | ADPRHL2 | 4.8 | 9.72E−06 | 0.44 | CIZ1 | 3.3 | 3.98E−03 | 0.15 | FBXO9 | 2.32 | 6.10E−02 | −0.12 |
| PES1 | 8 | 1.83E−14 | 0.83 | METTL2A | 4.8 | 9.73E−06 | 0.43 | RNF216 | 3.3 | 3.92E−03 | 0.13 | TPTE2 | 2.31 | 6.22E−02 | 0.94 |
| RPS29 | 8 | 1.84E−14 | 0.62 | CTNNBIP1 | 4.8 | 9.82E−06 | 0.25 | FBXL19 | 3.3 | 3.97E−03 | 0.12 | FLT3 | 2.31 | 6.31E−02 | 0.79 |
| TBCEL | 7.99 | 1.90E−14 | 4.3 | TRPC1 | 4.8 | 9.68E−06 | 0.23 | SLC22A5 | 3.3 | 3.96E−03 | 0.11 | SDSL | 2.31 | 6.34E−02 | 0.65 |
| STRAP | 7.99 | 1.94E−14 | 0.5 | GANAB | 4.8 | 1.00E−05 | 0.21 | MTO1 | 3.3 | 3.90E−03 | 0.09 | SLC15A3 | 2.31 | 6.21E−02 | 0.6 |
| MIR4435-2HG | 7.99 | 1.89E−14 | 0.31 | EPB41 | 4.8 | 9.98E−06 | 0.19 | MIB2 | 3.3 | 3.92E−03 | 0.09 | ZBTB8A | 2.31 | 6.23E−02 | 0.32 |
| HIRA | 7.98 | 2.10E−14 | 0.76 | STX12 | 4.8 | 9.68E−06 | 0.18 | DGCR8 | 3.3 | 3.93E−03 | 0.07 | TMEM37 | 2.31 | 6.35E−02 | 0.3 |
| TAF11 | 7.98 | 2.12E−14 | 0.52 | WIPF3 | 4.79 | 1.05E−05 | 0.85 | ANAPC7 | 3.3 | 3.91E−03 | 0.05 | LLPH | 2.31 | 6.28E−02 | 0.25 |
| NFIX | 7.98 | 2.11E−14 | 0.41 | DGKI | 4.79 | 1.02E−05 | 0.8 | SCARF2 | 3.29 | 4.10E−03 | 2.28 | CIT | 2.31 | 6.28E−02 | 0.16 |
| SCYL2 | 7.98 | 2.06E−14 | 0.4 | GRK4 | 4.79 | 1.02E−05 | 0.8 | MYOCD | 3.29 | 4.00E−03 | 1.54 | FAM19A2 | 2.31 | 6.28E−02 | 0.05 |
| TMBIM1 | 7.98 | 2.11E−14 | 0.38 | SLC22A4 | 4.79 | 1.01E−05 | 0.52 | PRRG2 | 3.29 | 4.08E−03 | 0.44 | UBXN11 | 2.31 | 6.26E−02 | 0.01 |
| RPS26 | 7.97 | 2.33E−14 | 1.54 | KLHL22 | 4.79 | 1.02E−05 | 0.34 | TMTC3 | 3.29 | 4.03E−03 | 0.15 | SLC37A3 | 2.31 | 6.33E−02 | −0.01 |
| TANGO2 | 7.97 | 2.31E−14 | 0.61 | RTKN | 4.79 | 1.06E−05 | 0.22 | TNFSF13B | 3.29 | 4.07E−03 | 0.14 | VSIG10 | 2.31 | 6.32E−02 | −0.04 |
| ADRM1 | 7.96 | 2.45E−14 | 1.01 | SOSTDC1 | 4.78 | 1.07E−05 | 5.14 | CYP27A1 | 3.29 | 4.09E−03 | 0.14 | MAGOH | 2.31 | 6.23E−02 | −0.07 |
| DYNLL1 | 7.96 | 2.56E−14 | 0.71 | ABCF2 | 4.78 | 1.11E−05 | 3 | NGDN | 3.29 | 4.02E−03 | 0.1 | CDC123 | 2.31 | 6.36E−02 | −0.09 |
| MME | 7.95 | 2.64E−14 | 1.46 | SLIT3 | 4.78 | 1.08E−05 | 1.21 | EIF2B1 | 3.29 | 4.05E−03 | 0.1 | CGB7 | 2.3 | 6.49E−02 | 2.04 |
| ASAP3 | 7.95 | 2.67E−14 | 1.32 | ZNF526 | 4.78 | 1.07E−05 | 1.11 | PRPF38B | 3.29 | 4.07E−03 | 0.1 | MORN2 | 2.3 | 6.42E−02 | 1.27 |
| ATP6V1G1 | 7.95 | 2.68E−14 | 0.85 | NHP2 | 4.78 | 1.10E−05 | 0.85 | SAP130 | 3.29 | 4.10E−03 | 0.05 | CTU2 | 2.3 | 6.40E−02 | 1.1 |
| FPGS | 7.94 | 2.86E−14 | 0.48 | DEXI | 4.78 | 1.09E−05 | 0.78 | DAPK2 | 3.29 | 4.07E−03 | 0.03 | RADIL | 2.3 | 6.49E−02 | 1.06 |
| KPNA1 | 7.93 | 3.22E−14 | 0.39 | NEXN | 4.78 | 1.09E−05 | 0.66 | RASSF8 | 3.29 | 4.09E−03 | −0.1 | TMEM115 | 2.3 | 6.51E−02 | 0.67 |
| UQCR10 | 7.92 | 3.29E−14 | 1.11 | FBRS | 4.78 | 1.07E−05 | 0.33 | ZNF699 | 3.28 | 4.21E−03 | 0.71 | CASKIN2 | 2.3 | 6.41E−02 | 0.6 |
| PPP1R13L | 7.92 | 3.42E−14 | 0.36 | PLOD1 | 4.78 | 1.07E−05 | 0.3 | COX6B2 | 3.28 | 4.19E−03 | 0.64 | FSIP1 | 2.3 | 6.44E−02 | 0.53 |
| XAF1 | 7.92 | 3.26E−14 | 0.35 | WBP2NL | 4.78 | 1.07E−05 | 0.26 | MED22 | 3.28 | 4.12E−03 | 0.58 | SPATA21 | 2.3 | 6.51E−02 | 0.52 |
| LMTK3 | 7.91 | 3.52E−14 | 0.74 | PNPLA2 | 4.78 | 1.09E−05 | 0.22 | RASSF8-AS1 | 3.28 | 4.12E−03 | 0.53 | EIF1AD | 2.3 | 6.50E−02 | 0.38 |
| UBL5 | 7.9 | 3.97E−14 | 1.16 | MPP3 | 4.78 | 1.11E−05 | 0.22 | CCDC97 | 3.28 | 4.15E−03 | 0.44 | NFKBID | 2.3 | 6.46E−02 | 0.14 |
| MT-ATP6 | 7.9 | 4.04E−14 | 0.46 | LMOD1 | 4.77 | 1.15E−05 | 2.24 | ZBTB21 | 3.28 | 4.12E−03 | 0.35 | LDB2 | 2.3 | 6.40E−02 | 0.08 |
| RHBDD2 | 7.89 | 4.32E−14 | 1.03 | ARHGEF26 | 4.77 | 1.14E−05 | 0.71 | CRTAP | 3.28 | 4.12E−03 | 0.28 | PRDM11 | 2.3 | 6.48E−02 | −0.04 |
| WDR66 | 7.89 | 4.44E−14 | 1 | STT3A | 4.77 | 1.15E−05 | 0.24 | FARSA | 3.28 | 4.17E−03 | 0.28 | ERCC1 | 2.3 | 6.45E−02 | −0.05 |
| LSM5 | 7.89 | 4.25E−14 | 0.85 | CRADD | 4.77 | 1.12E−05 | 0.21 | PIK3IP1 | 3.28 | 4.23E−03 | 0.27 | NFRKB | 2.3 | 6.48E−02 | −0.05 |
| CD151 | 7.88 | 4.64E−14 | 0.39 | INVS | 4.77 | 1.15E−05 | 0.2 | PRDM15 | 3.28 | 4.25E−03 | 0.24 | SYNGAP1 | 2.3 | 6.38E−02 | −0.09 |
| SLC1A5 | 7.87 | 5.02E−14 | 1.33 | PEX10 | 4.76 | 1.19E−05 | 0.74 | LRP10 | 3.28 | 4.18E−03 | 0.17 | MCF2L2 | 2.3 | 6.51E−02 | −0.28 |
| RYR2 | 7.87 | 5.03E−14 | 0.89 | LRCH4 | 4.76 | 1.18E−05 | 0.37 | FBXL12 | 3.28 | 4.23E−03 | 0.12 | MDGA1 | 2.29 | 6.54E−02 | 1.24 |
| ANKS3 | 7.86 | 5.59E−14 | 0.43 | EDC3 | 4.76 | 1.20E−05 | 0.26 | EEFSEC | 3.28 | 4.15E−03 | 0.08 | TEF | 2.29 | 6.65E−02 | 0.82 |
| AGGF1 | 7.85 | 5.80E−14 | 2.79 | LAD1 | 4.76 | 1.19E−05 | 0.25 | CNIH3 | 3.28 | 4.16E−03 | 0.07 | LINC01476 | 2.29 | 6.60E−02 | 0.76 |
| SBF2-AS1 | 7.85 | 5.95E−14 | 0.71 | OSTM1 | 4.76 | 1.22E−05 | 0.22 | EFHC1 | 3.28 | 4.23E−03 | 0.07 | CCNJL | 2.29 | 6.53E−02 | 0.74 |
| SNX19 | 7.85 | 5.88E−14 | 0.5 | AC010967.2 | 4.75 | 1.25E−05 | 3.06 | BRCC3 | 3.28 | 4.12E−03 | 0.04 | TCN1 | 2.29 | 6.57E−02 | 0.56 |
| RHBDF2 | 7.85 | 6.05E−14 | 0.45 | LINC01429 | 4.75 | 1.28E−05 | 1.61 | ILK | 3.28 | 4.12E−03 | 0.03 | C16orf95 | 2.29 | 6.65E−02 | 0.38 |
| TMEM192 | 7.84 | 6.40E−14 | 0.87 | DSCAM | 4.75 | 1.25E−05 | 1.22 | MORN3 | 3.27 | 4.33E−03 | 1.4 | ZFHX2 | 2.29 | 6.62E−02 | 0.34 |
| FBLN1 | 7.83 | 6.80E−14 | 1.4 | ATP6VIF | 4.75 | 1.25E−05 | 0.94 | POP7 | 3.27 | 4.36E−03 | 1.24 | UBE2D4 | 2.29 | 6.59E−02 | 0.22 |
| ALKBH6 | 7.83 | 6.79E−14 | 1.16 | PLBD2 | 4.75 | 1.23E−05 | 0.91 | KBTBD4 | 3.27 | 4.26E−03 | 1.08 | MAP3K10 | 2.29 | 6.58E−02 | 0.11 |
| TMEM30A | 7.83 | 6.63E−14 | 0.66 | ZNF768 | 4.75 | 1.23E−05 | 0.81 | MTRF1L | 3.27 | 4.38E−03 | 0.41 | TRIM65 | 2.29 | 6.61E−02 | 0.07 |
| LRRC58 | 7.83 | 6.98E−14 | 0.59 | ZNF281 | 4.75 | 1.27E−05 | 0.54 | ATMIN | 3.27 | 4.31E−03 | 0.28 | RBBP6 | 2.29 | 6.67E−02 | 0.02 |
| ELN | 7.82 | 7.21E−14 | 1.44 | FAM136A | 4.75 | 1.25E−05 | 0.38 | MYADM | 3.27 | 4.35E−03 | 0.25 | SRGAP2 | 2.29 | 6.57E−02 | 0.01 |
| MZT2A | 7.82 | 7.55E−14 | 0.75 | SUGCT | 4.75 | 1.28E−05 | 0.33 | DHRS1 | 3.27 | 4.38E−03 | 0.15 | METTL6 | 2.29 | 6.55E−02 | −0.06 |
| PPP6R1 | 7.82 | 7.23E−14 | 0.56 | LTBP4 | 4.75 | 1.26E−05 | 0.32 | TESK2 | 3.27 | 4.26E−03 | 0.14 | ASL | 2.29 | 6.66E−02 | −0.09 |
| CRTC3 | 7.82 | 7.22E−14 | 0.39 | CAPS | 4.75 | 1.23E−05 | 0.27 | CNNM2 | 3.27 | 4.25E−03 | 0.11 | GPR89A | 2.29 | 6.61E−02 | −0.14 |
| COPZ1 | 7.81 | 7.63E−14 | 0.57 | ZNF12 | 4.75 | 1.24E−05 | 0.23 | BOC | 3.26 | 4.51E−03 | 1.35 | AC009120.4 | 2.28 | 6.72E−02 | 1.38 |
| EI24 | 7.8 | 8.36E−14 | 0.74 | ICAM2 | 4.74 | 1.30E−05 | 1.14 | RARRES2 | 3.26 | 4.42E−03 | 1.22 | NPIPB6 | 2.28 | 6.71E−02 | 1.16 |
| PTDSS1 | 7.79 | 9.57E−14 | 0.75 | B3GAT2 | 4.74 | 1.33E−05 | 0.6 | RFX8 | 3.26 | 4.47E−03 | 1.12 | GUCA1B | 2.28 | 6.82E−02 | 0.78 |
| TLDC1 | 7.79 | 9.24E−14 | 0.53 | HDX | 4.74 | 1.30E−05 | 0.45 | DNASE2 | 3.26 | 4.40E−03 | 1.03 | PCBP3 | 2.28 | 6.71E−02 | 0.76 |
| GOS2 | 7.78 | 1.02E−13 | 2.21 | OBSL1 | 4.74 | 1.29E−05 | 0.41 | CYB561D1 | 3.26 | 4.46E−03 | 0.5 | APBB1 | 2.28 | 6.69E−02 | 0.71 |
| LINC01242 | 7.78 | 1.02E−13 | 2.1 | SPECC1 | 4.74 | 1.32E−05 | 0.38 | ARSB | 3.26 | 4.51E−03 | 0.5 | FBF1 | 2.28 | 6.75E−02 | 0.49 |
| KLF10 | 7.78 | 9.81E−14 | 0.74 | SIRT6 | 4.74 | 1.32E−05 | 0.24 | CNTNAP3 | 3.26 | 4.50E−03 | 0.45 | MADCAM1 | 2.28 | 6.77E−02 | 0.45 |
| PHTF1 | 7.78 | 9.83E−14 | 0.55 | TRIM31-AS1 | 4.74 | 1.30E−05 | 0.21 | AMZ2 | 3.26 | 4.45E−03 | 0.42 | LACTB2-AS1 | 2.28 | 6.74E−02 | 0.44 |
| HADHA | 7.78 | 9.91E−14 | 0.36 | EPB41L4A | 4.74 | 1.33E−05 | 0.19 | EPM2A | 3.26 | 4.51E−03 | 0.33 | PPP1R3E | 2.28 | 6.79E−02 | 0.37 |
| TRMT112 | 7.77 | 1.05E−13 | 1.47 | CPVL | 4.73 | 1.38E−05 | 1.14 | SLC35C1 | 3.26 | 4.41E−03 | 0.28 | SMAD7 | 2.28 | 6.78E−02 | 0.34 |
| ALDH1L1 | 7.77 | 1.11E−13 | 1.19 | KSR2 | 4.73 | 1.37E−05 | 0.8 | ELOVL1 | 3.26 | 4.48E−03 | 0.28 | MID1IP1 | 2.28 | 6.81E−02 | 0.32 |
| CD24 | 7.77 | 1.08E−13 | 0.53 | PQLC1 | 4.73 | 1.35E−05 | 0.38 | CHPF2 | 3.26 | 4.43E−03 | 0.23 | NIP7 | 2.28 | 6.82E−02 | 0.26 |
| AES | 7.77 | 1.06E−13 | 0.43 | SP110 | 4.73 | 1.40E−05 | 0.33 | GRHPR | 3.26 | 4.44E−03 | 0.14 | KPNA4 | 2.28 | 6.70E−02 | 0.09 |
| PSMB5 | 7.76 | 1.16E−13 | 0.73 | ZNF717 | 4.73 | 1.38E−05 | 0.24 | RFNG | 3.26 | 4.43E−03 | 0.12 | BBX | 2.28 | 6.67E−02 | 0.08 |
| NFKBIZ | 7.76 | 1.18E−13 | 0.4 | ZBTB8OS | 4.73 | 1.37E−05 | 0.2 | LIN7C | 3.26 | 4.51E−03 | 0.12 | PPP2R5D | 2.28 | 6.71E−02 | 0.05 |
| MSRB3 | 7.75 | 1.27E−13 | 1.21 | CXCL16 | 4.72 | 1.47E−05 | 1.09 | ENOPH1 | 3.26 | 4.45E−03 | 0.11 | TMEM232 | 2.28 | 6.75E−02 | 0.05 |
| ZNF500 | 7.75 | 1.25E−13 | 1.05 | IKBIP | 4.72 | 1.41E−05 | 0.95 | NSUN4 | 3.26 | 4.46E−03 | 0.07 | ZNF155 | 2.28 | 6.68E−02 | 0.04 |
| ZNF345 | 7.75 | 1.30E−13 | 0.68 | JOSD2 | 4.72 | 1.43E−05 | 0.69 | PPFIBP2 | 3.26 | 4.43E−03 | 0.01 | CCT6A | 2.28 | 6.80E−02 | −0.01 |
| CANX | 7.75 | 1.25E−13 | 0.43 | POLDIP2 | 4.72 | 1.43E−05 | 0.56 | DPM1 | 3.26 | 4.51E−03 | −0.01 | WDR4 | 2.28 | 6.68E−02 | −0.05 |
| PRRC2A | 7.74 | 1.33E−13 | 0.57 | GLTP | 4.72 | 1.46E−05 | 0.49 | PHGDH | 3.25 | 4.57E−03 | 1.19 | RCAN3 | 2.28 | 6.79E−02 | −0.1 |
| GCNT2 | 7.73 | 1.41E−13 | 0.51 | PPP1R26-AS1 | 4.72 | 1.43E−05 | 0.39 | MAP4K2 | 3.25 | 4.56E−03 | 0.65 | FAM86C1 | 2.27 | 6.97E−02 | 0.85 |
| ALDH3B1 | 7.72 | 1.61E−13 | 0.65 | UBFD1 | 4.72 | 1.46E−05 | 0.36 | NDUFA8 | 3.25 | 4.55E−03 | 0.51 | CSF1 | 2.27 | 6.90E−02 | 0.71 |
| RUNX2 | 7.72 | 1.59E−13 | 0.58 | DUS1L | 4.72 | 1.42E−05 | 0.29 | FAM66D | 3.25 | 4.60E−03 | 0.49 | TMEM132B | 2.27 | 6.89E−02 | 0.7 |
| PPIB | 7.71 | 1.73E−13 | 0.92 | PSMC1 | 4.72 | 1.43E−05 | 0.28 | SYT12 | 3.25 | 4.55E−03 | 0.37 | VASN | 2.27 | 6.85E−02 | 0.49 |
| ZNF189 | 7.71 | 1.76E−13 | 0.71 | NR2F6 | 4.72 | 1.43E−05 | 0.27 | SCPEP1 | 3.25 | 4.57E−03 | 0.36 | DPM3 | 2.27 | 6.97E−02 | 0.36 |
| FBXO28 | 7.71 | 1.73E−13 | 0.43 | STYXL1 | 4.72 | 1.41E−05 | 0.21 | LINC00342 | 3.25 | 4.65E−03 | 0.34 | ZNF268 | 2.27 | 6.97E−02 | 0.29 |
| BAIAP2 | 7.71 | 1.71E−13 | 0.39 | CMC2 | 4.72 | 1.44E−05 | 0.21 | SPRY2 | 3.25 | 4.57E−03 | 0.33 | ADGRF1 | 2.27 | 6.85E−02 | 0.2 |
| NPAS2 | 7.71 | 1.76E−13 | 0.25 | EEF1B2 | 4.72 | 1.42E−05 | 0.13 | RBMX2 | 3.25 | 4.58E−03 | 0.2 | SLC9B1 | 2.27 | 6.83E−02 | 0.18 |
| TLL1 | 7.7 | 1.80E−13 | 1.82 | CSAD | 4.72 | 1.43E−05 | 0.13 | SDF2 | 3.25 | 4.54E−03 | 0.19 | NUDT16L1 | 2.27 | 6.83E−02 | 0.16 |
| HYOU1 | 7.7 | 1.84E−13 | 1.41 | CAPN3 | 4.71 | 1.49E−05 | 4.26 | AGAP1 | 3.25 | 4.56E−03 | 0.1 | KIAA0895L | 2.27 | 6.95E−02 | 0.16 |
| SEC61G | 7.7 | 1.85E−13 | 1.26 | CYBRD1 | 4.71 | 1.50E−05 | 0.35 | PLPP5 | 3.25 | 4.60E−03 | 0.02 | GAS2L1 | 2.27 | 6.85E−02 | 0.14 |
| LIMK1 | 7.7 | 1.87E−13 | 0.82 | RABEP2 | 4.71 | 1.53E−05 | 0.22 | PDZD2 | 3.25 | 4.55E−03 | 0.01 | AREG | 2.27 | 6.92E−02 | 0.11 |
| P2RX4 | 7.7 | 1.78E−13 | 0.66 | KPNA3 | 4.71 | 1.50E−05 | 0.18 | Z83844.1 | 3.24 | 4.79E−03 | 2.45 | INTS6-AS1 | 2.27 | 6.96E−02 | 0.11 |
| BCL7C | 7.7 | 1.88E−13 | 0.58 | C11orf58 | 4.71 | 1.48E−05 | 0.16 | STXBP5L | 3.24 | 4.81E−03 | 1.14 | UVRAG | 2.27 | 6.92E−02 | 0.1 |
| PCYT1A | 7.7 | 1.84E−13 | 0.56 | SYN3 | 4.7 | 1.58E−05 | 0.9 | KDELR3 | 3.24 | 4.69E−03 | 0.69 | TVP23B | 2.27 | 6.94E−02 | 0.07 |
| VDR | 7.7 | 1.85E−13 | 0.51 | AURKAIP1 | 4.7 | 1.58E−05 | 0.73 | CNPPD1 | 3.24 | 4.79E−03 | 0.65 | UXT | 2.27 | 6.97E−02 | 0.06 |
| ATR | 7.7 | 1.85E−13 | 0.34 | UQCC2 | 4.7 | 1.60E−05 | 0.57 | EBF4 | 3.24 | 4.72E−03 | 0.52 | SRC | 2.27 | 6.95E−02 | 0.03 |
| P3H2 | 7.7 | 1.85E−13 | 0.34 | DAD1 | 4.7 | 1.61E−05 | 0.26 | TBL2 | 3.24 | 4.77E−03 | 0.46 | DCTN4 | 2.27 | 6.98E−02 | 0.02 |
| MRPS11 | 7.69 | 2.00E−13 | 0.86 | SF3B6 | 4.7 | 1.54E−05 | 0.19 | CBX7 | 3.24 | 4.76E−03 | 0.44 | FAM96A | 2.27 | 6.90E−02 | 0 |
| ZNF600 | 7.69 | 1.95E−13 | 0.69 | ARSA | 4.69 | 1.68E−05 | 0.51 | PPP1R8 | 3.24 | 4.69E−03 | 0.3 | MYO1C | 2.27 | 6.94E−02 | −0.01 |
| EFCAB3 | 7.68 | 2.15E−13 | 1.72 | STK19 | 4.69 | 1.62E−05 | 0.5 | PGLS | 3.24 | 4.73E−03 | 0.13 | MOB3A | 2.27 | 6.97E−02 | −0.01 |
| ENG | 7.68 | 2.08E−13 | 1.01 | CISD2 | 4.69 | 1.62E−05 | 0.49 | BAZ1B | 3.24 | 4.74E−03 | 0.1 | OTUD5 | 2.27 | 6.83E−02 | −0.03 |
| PSMA7 | 7.68 | 2.08E−13 | 0.61 | SLIT2 | 4.69 | 1.68E−05 | 0.39 | CYTH2 | 3.24 | 4.83E−03 | 0.1 | SIRT3 | 2.27 | 6.92E−02 | −0.03 |
| PTPN1 | 7.68 | 2.19E−13 | 0.42 | PER3 | 4.69 | 1.67E−05 | 0.33 | ADAM15 | 3.24 | 4.80E−03 | 0.09 | GTPBP2 | 2.27 | 6.83E−02 | −0.04 |
| CLEC16A | 7.68 | 2.17E−13 | 0.37 | SPAG17 | 4.69 | 1.63E−05 | 0.32 | FKBP9 | 3.24 | 4.72E−03 | 0.06 | LNX1 | 2.27 | 6.87E−02 | −0.04 |
| NDUFA12 | 7.67 | 2.30E−13 | 0.87 | MED21 | 4.69 | 1.67E−05 | 0.3 | SZT2 | 3.24 | 4.77E−03 | 0.05 | TUBE1 | 2.27 | 6.91E−02 | −0.07 |
| SDF4 | 7.67 | 2.31E−13 | 0.77 | ROR1 | 4.69 | 1.62E−05 | 0.24 | SYBU | 3.24 | 4.83E−03 | 0.05 | EOGT | 2.27 | 6.87E−02 | −0.12 |
| IQCJ-SCHIP1 | 7.66 | 2.49E−13 | 0.63 | VPS33B | 4.68 | 1.69E−05 | 3.06 | GTF2H1 | 3.24 | 4.83E−03 | 0.04 | UGT2B15 | 2.26 | 7.01E−02 | 1.53 |
| NR2C1 | 7.66 | 2.51E−13 | 0.4 | TUBB8 | 4.68 | 1.70E−05 | 1.23 | TSPAN10 | 3.23 | 4.89E−03 | 1.21 | TIMM8A | 2.26 | 7.04E−02 | 0.91 |
| EIF1 | 7.66 | 2.41E−13 | 0.39 | NPIPB3 | 4.68 | 1.70E−05 | 1.14 | MS4A8 | 3.23 | 4.84E−03 | 0.38 | DOLPP1 | 2.26 | 7.10E−02 | 0.74 |
| EPS15L1 | 7.66 | 2.49E−13 | 0.39 | PALM3 | 4.68 | 1.71E−05 | 0.85 | WFDC10B | 3.23 | 4.92E−03 | 0.29 | COL25A1 | 2.26 | 7.11E−02 | 0.64 |
| PRDX5 | 7.65 | 2.74E−13 | 0.73 | TRPV4 | 4.68 | 1.76E−05 | 0.27 | TTLL11 | 3.23 | 4.84E−03 | 0.19 | DLL1 | 2.26 | 7.02E−02 | 0.58 |
| NAT9 | 7.65 | 2.68E−13 | 0.62 | ZNF766 | 4.68 | 1.72E−05 | 0.26 | TIMM44 | 3.23 | 4.99E−03 | 0.17 | PPFIA2 | 2.26 | 7.06E−02 | 0.4 |
| SNU13 | 7.65 | 2.66E−13 | 0.58 | B3GNT3 | 4.68 | 1.72E−05 | 0.25 | LSM1 | 3.23 | 4.93E−03 | 0.15 | ADIRF | 2.26 | 7.11E−02 | 0.4 |
| PILRB | 7.65 | 2.64E−13 | 0.47 | RING1 | 4.68 | 1.74E−05 | 0.17 | CTPS1 | 3.23 | 4.92E−03 | 0.08 | MORC2-AS1 | 2.26 | 7.07E−02 | 0.34 |
| NDUFA3 | 7.64 | 2.90E−13 | 1.99 | PRSS22 | 4.68 | 1.75E−05 | 0.15 | NMNAT1 | 3.23 | 4.88E−03 | 0.06 | SLC48A1 | 2.26 | 7.06E−02 | 0.11 |
| ZYX | 7.64 | 2.78E−13 | 0.74 | C2orf81 | 4.67 | 1.80E−05 | 4.21 | SMAGP | 3.23 | 4.85E−03 | 0.03 | 9-Mar | 2.26 | 7.13E−02 | 0.08 |
| NMRAL1 | 7.64 | 2.97E−13 | 0.54 | 1-Sep | 4.67 | 1.80E−05 | 1.63 | PFDN1 | 3.23 | 4.98E−03 | 0.01 | ZNF322 | 2.26 | 7.08E−02 | 0.07 |
| SLC9A3R1 | 7.63 | 3.05E−13 | 0.82 | CRABP2 | 4.67 | 1.83E−05 | 1.47 | ZNF385C | 3.22 | 5.10E−03 | 1.39 | SULT1B1 | 2.26 | 7.03E−02 | 0.06 |
| FGF19 | 7.61 | 3.65E−13 | 3.44 | TSPAN2 | 4.67 | 1.85E−05 | 1.15 | TMEM44-AS1 | 3.22 | 5.03E−03 | 0.5 | TP53 | 2.26 | 7.08E−02 | 0.03 |
| MED17 | 7.61 | 3.57E−13 | 2.18 | CDSN | 4.67 | 1.81E−05 | 0.61 | PPP1R18 | 3.22 | 5.10E−03 | 0.29 | CABIN1 | 2.26 | 6.99E−02 | 0.02 |
| CACNA1A | 7.61 | 3.59E−13 | 1.19 | TRAFD1 | 4.67 | 1.82E−05 | 0.44 | LMCD1 | 3.22 | 5.03E−03 | 0.25 | CARD6 | 2.26 | 7.10E−02 | 0.01 |
| COL6A3 | 7.6 | 3.84E−13 | 0.57 | BACH2 | 4.67 | 1.83E−05 | 0.43 | ANKRD42 | 3.22 | 5.09E−03 | 0.08 | SAMM50 | 2.26 | 7.10E−02 | 0.01 |
| PHC2 | 7.6 | 3.79E−13 | 0.45 | ARID3A | 4.67 | 1.79E−05 | 0.42 | MTF2 | 3.22 | 5.00E−03 | 0.07 | PPP1R12C | 2.26 | 7.13E−02 | 0 |
| DDB1 | 7.6 | 3.94E−13 | 0.43 | COL5A1 | 4.67 | 1.84E−05 | 0.42 | TIMM17A | 3.22 | 5.07E−03 | 0.05 | HUS1 | 2.26 | 7.05E−02 | −0.01 |
| MGRN1 | 7.6 | 3.94E−13 | 0.41 | KIF1C | 4.67 | 1.79E−05 | 0.3 | APIM1 | 3.22 | 5.14E−03 | 0.04 | TRAPPC13 | 2.26 | 7.01E−02 | −0.02 |
| PDIA6 | 7.59 | 4.32E−13 | 0.56 | WDTC1 | 4.67 | 1.77E−05 | 0.25 | TICAM1 | 3.22 | 5.04E−03 | 0.02 | GATA4 | 2.26 | 7.07E−02 | −0.02 |
| RPL4 | 7.59 | 4.20E−13 | 0.47 | SMARCAL1 | 4.67 | 1.83E−05 | 0.22 | BRD2 | 3.22 | 5.00E−03 | 0 | ERI1 | 2.26 | 7.10E−02 | −0.02 |
| ENDOV | 7.58 | 4.55E−13 | 0.77 | KLHL7 | 4.67 | 1.82E−05 | 0.2 | RUNX3 | 3.21 | 5.21E−03 | 1.51 | PPARGC1B | 2.26 | 7.07E−02 | −0.06 |
| BCAR1 | 7.58 | 4.55E−13 | 0.41 | UEVLD | 4.67 | 1.79E−05 | 0.19 | TMEM19 | 3.21 | 5.33E−03 | 0.67 | CCT5 | 2.26 | 7.11E−02 | −0.07 |
| EIF1AX | 7.58 | 4.62E−13 | 0.41 | UBXN2A | 4.67 | 1.79E−05 | 0.18 | BLOC1S4 | 3.21 | 5.17E−03 | 0.65 | GRK7 | 2.25 | 7.18E−02 | 1.7 |
| GGPS1 | 7.57 | 4.89E−13 | 2.88 | ZNF532 | 4.67 | 1.82E−05 | 0.18 | PEX16 | 3.21 | 5.30E−03 | 0.48 | SLC4A10 | 2.25 | 7.17E−02 | 0.94 |
| TMEM173 | 7.57 | 4.77E−13 | 0.96 | UBE2M | 4.66 | 1.88E−05 | 0.75 | REC8 | 3.21 | 5.22E−03 | 0.43 | NDUFAF1 | 2.25 | 7.14E−02 | 0.31 |
| PAAF1 | 7.57 | 4.72E−13 | 0.61 | FAM120AOS | 4.66 | 1.92E−05 | 0.4 | CNOT3 | 3.21 | 5.30E−03 | 0.26 | BNIP1 | 2.25 | 7.27E−02 | 0.29 |
| ZNF608 | 7.57 | 5.03E−13 | 0.39 | LINC00667 | 4.66 | 1.94E−05 | 0.36 | TBC1D13 | 3.21 | 5.29E−03 | 0.25 | MTCP1 | 2.25 | 7.24E−02 | 0.24 |
| CMTR1 | 7.56 | 5.18E−13 | 0.71 | FUCA2 | 4.66 | 1.89E−05 | 0.33 | KCTD5 | 3.21 | 5.31E−03 | 0.23 | FTSJ3 | 2.25 | 7.19E−02 | 0.15 |
| NSFL1C | 7.56 | 5.30E−13 | 0.47 | CNKSR1 | 4.66 | 1.92E−05 | 0.28 | WDR60 | 3.21 | 5.30E−03 | 0.15 | COL18A1 | 2.25 | 7.17E−02 | 0 |
| MEF2C | 7.55 | 5.75E−13 | 0.97 | CMAS | 4.66 | 1.86E−05 | 0.19 | ZBED4 | 3.21 | 5.20E−03 | 0.07 | RAB13 | 2.25 | 7.16E−02 | −0.02 |
| RABL2A | 7.55 | 5.61E−13 | 0.81 | DDX21 | 4.66 | 1.89E−05 | 0.15 | PSMD13 | 3.21 | 5.16E−03 | 0.06 | APIP | 2.25 | 7.21E−02 | −0.04 |
| SLC9A7 | 7.55 | 5.47E−13 | 0.52 | B4GALNT2 | 4.65 | 1.98E−05 | 1.52 | SDHAF1 | 3.2 | 5.37E−03 | 1.32 | SACS | 2.25 | 7.16E−02 | −0.09 |
| GPR176 | 7.54 | 6.22E−13 | 1.21 | FAHD2A | 4.65 | 1.97E−05 | 0.55 | CDH18 | 3.2 | 5.36E−03 | 1.25 | SLC18B1 | 2.25 | 7.19E−02 | −0.09 |
| TMEM106A | 7.54 | 5.87E−13 | 1.04 | KREMEN1 | 4.55 | 1.97E−05 | 0.48 | TMEM101 | 3.2 | 5.48E−03 | 0.71 | THAP4 | 2.25 | 7.26E−02 | −0.11 |
| MRPL33 | 7.53 | 6.45E−13 | 0.71 | PHF20 | 4.65 | 1.98E−05 | 0.2 | ZNF524 | 3.2 | 5.47E−03 | 0.68 | GINM1 | 2.25 | 7.15E−02 | −0.14 |
| AGPAT2 | 7.53 | 6.76E−13 | 0.63 | SNX3 | 4.65 | 1.98E−05 | 0.14 | PLXNC1 | 3.2 | 5.50E−03 | 0.35 | SMTN | 2.25 | 7.23E−02 | −0.14 |
| ASB7 | 7.53 | 6.70E−13 | 0.54 | SPTBN4 | 4.64 | 2.05E−05 | 1.17 | CADM2 | 3.2 | 5.34E−03 | 0.19 | PPIL4 | 2.25 | 7.29E−02 | −0.17 |
| TAGLN2 | 7.53 | 6.54E−13 | 0.46 | STPG1 | 4.54 | 2.06E−05 | 0.53 | CCDC122 | 3.2 | 5.42E−03 | 0.13 | SHISA6 | 2.24 | 7.31E−02 | 0.98 |
| SYNP0 | 7.52 | 6.97E−13 | 0.73 | LAMTOR1 | 4.64 | 2.05E−05 | 0.41 | EHHADH | 3.2 | 5.35E−03 | 0.09 | TIMM10 | 2.24 | 7.42E−02 | 0.92 |
| TAP1 | 7.52 | 6.99E−13 | 0.7 | EID1 | 4.64 | 2.04E−05 | 0.32 | AKIRIN1 | 3.2 | 5.48E−03 | −0.07 | FAT3 | 2.24 | 7.38E−02 | 0.74 |
| AP5Z1 | 7.52 | 7.23E−13 | 0.6 | NECAP2 | 4.64 | 2.06E−05 | 0.29 | CCDC178 | 3.19 | 5.55E−03 | 1.42 | ZNF749 | 2.24 | 7.35E−02 | 0.72 |
| NUDC | 7.52 | 6.97E−13 | 0.53 | KITLG | 4.64 | 2.12E−05 | 0.29 | MIR3681HG | 3.19 | 5.55E−03 | 0.95 | CHCHD4 | 2.24 | 7.46E−02 | 0.71 |
| PPP4C | 7.52 | 6.81E−13 | 0.49 | ATP2A2 | 4.64 | 2.09E−05 | 0.21 | SGF29 | 3.19 | 5.55E−03 | 0.35 | MMEL1 | 2.24 | 7.40E−02 | 0.57 |
| HLA-E | 7.52 | 7.05E−13 | 0.46 | TMX4 | 4.64 | 2.06E−05 | 0.2 | THOC5 | 3.19 | 5.54E−03 | 0.32 | NABP2 | 2.24 | 7.46E−02 | 0.49 |
| HPS5 | 7.52 | 7.27E−13 | 0.45 | ANKFY1 | 4.54 | 2.12E−05 | 0.18 | EMD | 3.19 | 5.64E−03 | 0.19 | ZNF563 | 2.24 | 7.34E−02 | 0.44 |
| CEP290 | 7.52 | 7.14E−13 | 0.38 | NAALADL2 | 4.64 | 2.12E−05 | 0.15 | SLC36A1 | 3.19 | 5.57E−03 | 0.18 | LINC00598 | 2.24 | 7.36E−02 | 0.31 |
| OAS3 | 7.51 | 7.45E−13 | 1.33 | PAQR7 | 4.63 | 2.13E−05 | 0.98 | ZNF566 | 3.19 | 5.57E−03 | 0.16 | MRPS14 | 2.24 | 7.42E−02 | 0.28 |
| TBC1D16 | 7.51 | 7.46E−13 | 0.47 | NKAIN2 | 4.63 | 2.13E−05 | 0.72 | MAST3 | 3.19 | 5.57E−03 | 0.13 | RHOBTB2 | 2.24 | 7.42E−02 | 0.22 |
| TRIOBP | 7.51 | 7.79E−13 | 0.39 | PARVA | 4.63 | 2.14E−05 | 0.2 | RAB5B | 3.19 | 5.55E−03 | 0.12 | TMEM64 | 2.24 | 7.42E−02 | 0.1 |
| NFAT5 | 7.51 | 7.61E−13 | 0.19 | VPS37B | 4.63 | 2.13E−05 | 0.19 | SH2B1 | 3.19 | 5.59E−03 | 0.11 | FXYD3 | 2.24 | 7.41E−02 | 0.08 |
| AP2S1 | 7.5 | 8.26E−13 | 0.92 | MIR181A1HG | 4.63 | 2.18E−05 | 0.11 | SNF8 | 3.19 | 5.66E−03 | 0.11 | C3orf67 | 2.24 | 7.44E−02 | 0.06 |
| TAZ | 7.5 | 8.02E−13 | 0.52 | CARMN | 4.62 | 2.29E−05 | 1.2 | FAXDC2 | 3.19 | 5.60E−03 | 0.05 | MARVELD3 | 2.24 | 7.31E−02 | 0.04 |
| RTF1 | 7.5 | 8.06E−13 | 0.47 | EFEMP1 | 4.62 | 2.25E−05 | 1.04 | UBLCP1 | 3.19 | 5.68E−03 | 0.04 | RORC | 2.24 | 7.34E−02 | 0.04 |
| DCBLD1 | 7.5 | 8.32E−13 | 0.41 | ST3GAL5 | 4.62 | 2.27E−05 | 0.83 | PRRG4 | 3.19 | 5.59E−03 | 0 | RAP2B | 2.24 | 7.37E−02 | 0 |
| MTCH1 | 7.5 | 8.15E−13 | 0.16 | FBXL6 | 4.62 | 2.30E−05 | 0.63 | ZNF474 | 3.18 | 5.76E−03 | 5.06 | USP13 | 2.24 | 7.34E−02 | −0.03 |
| GDE1 | 7.49 | 8.48E−13 | 0.59 | CD109 | 4.62 | 2.24E−05 | 0.39 | TUBA8 | 3.18 | 5.77E−03 | 1.17 | UFL1 | 2.24 | 7.36E−02 | −0.06 |
| CA5B | 7.49 | 8.42E−13 | 0.46 | FSTL3 | 4.62 | 2.24E−05 | 0.34 | VCAM1 | 3.18 | 5.77E−03 | 1.05 | FGFR1OP | 2.24 | 7.37E−02 | −0.1 |
| PDLIM4 | 7.48 | 9.61E−13 | 1.85 | ANKRD49 | 4.62 | 2.24E−05 | 0.3 | RMI2 | 3.18 | 5.86E−03 | 0.84 | MPHOSPH6 | 2.24 | 7.42E−02 | −0.13 |
| CHMP3 | 7.48 | 9.52E−13 | 0.94 | ACAD9 | 4.62 | 2.23E−05 | 0.23 | MUL1 | 3.18 | 5.70E−03 | 0.83 | CEACAM7 | 2.23 | 7.63E−02 | 1.25 |
| DYNLRB1 | 7.48 | 9.19E−13 | 0.51 | PLAUR | 4.62 | 2.31E−05 | 0.19 | SYF2 | 3.18 | 5.82E−03 | 0.34 | LINC00535 | 2.23 | 7.48E−02 | 0.89 |
| UBE2L3 | 7.48 | 9.57E−13 | 0.42 | R3HDM1 | 4.62 | 2.32E−05 | 0.19 | PMS2 | 3.18 | 5.85E−03 | 0.21 | FUNDC1 | 2.23 | 7.47E−02 | 0.81 |
| NAA50 | 7.47 | 1.02E−12 | 0.38 | ABCD1 | 4.61 | 2.42E−05 | 0.71 | PPIH | 3.18 | 5.83E−03 | 0.2 | DOK1 | 2.23 | 7.58E−02 | 0.64 |
| PPP2RIA | 7.46 | 1.07E−12 | 0.53 | CNPY3 | 4.61 | 2.42E−05 | 0.69 | XRRA1 | 3.18 | 5.82E−03 | 0.09 | GPATCH3 | 2.23 | 7.52E−02 | 0.52 |
| ZNF302 | 7.46 | 1.07E−12 | 0.41 | DDT | 4.61 | 2.34E−05 | 0.59 | WIPF1 | 3.18 | 5.79E−03 | 0.08 | FRMD6-AS1 | 2.23 | 7.54E−02 | 0.5 |
| SNCG | 7.45 | 1.19E−12 | 1.47 | HSD11B2 | 4.61 | 2.35E−05 | 0.49 | ZNF714 | 3.18 | 5.69E−03 | 0.04 | CRB3 | 2.23 | 7.50E−02 | 0.41 |
| MTHFDIL | 7.45 | 1.17E−12 | 0.32 | ECHS1 | 4.61 | 2.41E−05 | 0.4 | TSPO | 3.18 | 5.69E−03 | −0.01 | NXPE3 | 2.23 | 7.56E−02 | 0.41 |
| IL20RB | 7.44 | 1.23E−12 | 0.55 | NFKBIB | 4.61 | 2.42E−05 | 0.38 | RBFOX3 | 3.17 | 6.04E−03 | 1.33 | EPB41L4A-AS1 | 2.23 | 7.50E−02 | 0.3 |
| DAP | 7.44 | 1.29E−12 | 0.45 | PI4KA | 4.61 | 2.37E−05 | 0.18 | MILR1 | 3.17 | 5.87E−03 | 1.07 | MYRFL | 2.23 | 7.63E−02 | 0.18 |
| KIAA0825 | 7.44 | 1.30E−12 | 0.42 | KCNN4 | 4.61 | 2.42E−05 | 0.18 | FAM53A | 3.17 | 5.97E−03 | 0.68 | SPON1 | 2.23 | 7.60E−02 | 0.12 |
| ANKLE2 | 7.44 | 1.27E−12 | 0.33 | TOP3B | 4.6 | 2.52E−05 | 0.66 | SERTAD1 | 3.17 | 5.96E−03 | 0.59 | NFATC2 | 2.23 | 7.48E−02 | 0.1 |
| CXorf38 | 7.43 | 1.39E−12 | 0.65 | SF3A2 | 4.6 | 2.44E−05 | 0.59 | EML6 | 3.17 | 5.94E−03 | 0.33 | ARAF | 2.23 | 7.52E−02 | 0.1 |
| TSC2 | 7.43 | 1.36E−12 | 0.3 | C9orf78 | 4.6 | 2.51E−05 | 0.45 | ZNF836 | 3.17 | 6.00E−03 | 0.31 | PTTG1IP | 2.23 | 7.47E−02 | 0.06 |
| FADS3 | 7.42 | 1.44E−12 | 1.33 | BOK | 4.6 | 2.51E−05 | 0.45 | MRPL57 | 3.17 | 5.88E−03 | 0.26 | SLC36A4 | 2.23 | 7.59E−02 | 0.02 |
| SKIV2L | 7.42 | 1.46E−12 | 0.9 | ECI2 | 4.6 | 2.47E−05 | 0.44 | DNAJC22 | 3.17 | 6.05E−03 | 0.21 | KIF3B | 2.23 | 7.55E−02 | 0 |
| WDR78 | 7.42 | 1.42E−12 | 0.68 | KHDC1 | 4.6 | 2.51E−05 | 0.37 | S100A16 | 3.17 | 5.87E−03 | 0.12 | HDDC2 | 2.23 | 7.58E−02 | −0.01 |
| RPS3A | 7.42 | 1.48E−12 | 0.61 | LYRM2 | 4.6 | 2.51E−05 | 0.31 | YWHAG | 3.17 | 5.89E−03 | 0.08 | MRPL45 | 2.23 | 7.56E−02 | −0.03 |
| TMEM63B | 7.42 | 1.42E−12 | 0.44 | SIRT2 | 4.6 | 2.46E−05 | 0.28 | MRRF | 3.17 | 5.94E−03 | 0.08 | TTF2 | 2.23 | 7.63E−02 | −0.04 |
| NUCB2 | 7.41 | 1.55E−12 | 0.84 | WDR35 | 4.6 | 2.48E−05 | 0.25 | NEK6 | 3.17 | 5.92E−03 | 0.06 | GNS | 2.23 | 7.52E−02 | −0.05 |
| PGP | 7.41 | 1.58E−12 | 0.58 | GIT2 | 4.6 | 2.47E−05 | 0.2 | TSTD2 | 3.17 | 6.04E−03 | 0.05 | LTBP1 | 2.23 | 7.57E−02 | −0.07 |
| MYDGF | 7.41 | 1.63E−12 | 0.55 | RUBCN | 4.6 | 2.50E−05 | 0.2 | RRN3 | 3.17 | 5.98E−03 | −0.03 | FAM20B | 2.23 | 7.47E−02 | −0.09 |
| MAP2 | 7.41 | 1.58E−12 | 0.49 | ATG13 | 4.6 | 2.50E−05 | 0.17 | SSB | 3.17 | 5.96E−03 | −0.04 | RASGRF2 | 2.23 | 7.61E−02 | −0.11 |
| BCAR3 | 7.4 | 1.65E−12 | 0.34 | LASP1 | 4.6 | 2.54E−05 | 0.17 | ZCCHC24 | 3.16 | 6.24E−03 | 0.36 | SNX16 | 2.23 | 7.47E−02 | −0.27 |
| IFI27L2 | 7.39 | 1.88E−12 | 1.55 | PSME3 | 4.59 | 2.59E−05 | 0.54 | TAF13 | 3.16 | 6.06E−03 | 0.32 | GPR1 | 2.22 | 7.71E−02 | 1.65 |
| AKAP6 | 7.39 | 1.80E−12 | 1.08 | LINC00649 | 4.59 | 2.57E−05 | 0.47 | MEGF8 | 3.16 | 6.07E−03 | 0.32 | UNC80 | 2.22 | 7.80E−02 | 1.41 |
| UQCRB | 7.39 | 1.82E−12 | 0.74 | MUC4 | 4.59 | 2.66E−05 | 0.41 | DUSP23 | 3.16 | 6.13E−03 | 0.25 | CD38 | 2.22 | 7.68E−02 | 1.36 |
| LAPTM4A | 7.39 | 1.81E−12 | 0.64 | RBM8A | 4.59 | 2.58E−05 | 0.37 | NUDT22 | 3.16 | 6.07E−03 | 0.15 | ADO | 2.22 | 7.69E−02 | 0.47 |
| MED10 | 7.38 | 1.94E−12 | 0.52 | MVB12A | 4.59 | 2.62E−05 | 0.28 | COL9A2 | 3.16 | 6.07E−03 | 0.15 | ZNF22 | 2.22 | 7.77E−02 | 0.46 |
| PLIN3 | 7.38 | 1.98E−12 | 0.5 | CSRNP3 | 4.59 | 2.60E−05 | 0.24 | LINC01184 | 3.16 | 6.16E−03 | 0.1 | TTLL10 | 2.22 | 7.72E−02 | 0.44 |
| CALM3 | 7.36 | 2.28E−12 | 1.09 | TRNAU1AP | 4.59 | 2.58E−05 | 0.19 | BCR | 3.16 | 6.20E−03 | 0.1 | CKMT1B | 2.22 | 7.65E−02 | 0.35 |
| CACNB1 | 7.36 | 2.31E−12 | 0.99 | TCEAL8 | 4.58 | 2.75E−05 | 1.14 | KIAA1328 | 3.16 | 6.06E−03 | 0.09 | DUOXA1 | 2.22 | 7.72E−02 | 0.32 |
| MARS | 7.36 | 2.31E−12 | 0.63 | DDIT3 | 4.58 | 2.77E−05 | 0.56 | HSDL2 | 3.16 | 6.18E−03 | 0.06 | XRCC1 | 2.22 | 7.64E−02 | 0.31 |
| DHX57 | 7.36 | 2.19E−12 | 0.5 | KCNMB3 | 4.58 | 2.71E−05 | 0.27 | NUDT21 | 3.16 | 6.19E−03 | 0.03 | ESPN | 2.22 | 7.69E−02 | 0.29 |
| MAPK14 | 7.36 | 2.28E−12 | 0.36 | BCL9L | 4.58 | 2.68E−05 | 0.24 | CRIP1 | 3.15 | 6.32E−03 | 2.26 | LRRC29 | 2.22 | 7.66E−02 | 0.24 |
| SPCS2 | 7.35 | 2.52E−12 | 0.44 | DPY30 | 4.58 | 2.76E−05 | 0.24 | ZNF382 | 3.15 | 6.33E−03 | 1.59 | PDE10A | 2.22 | 7.78E−02 | 0.08 |
| RPL39 | 7.34 | 2.58E−12 | 1.6 | PIGM | 4.57 | 2.79E−05 | 3.21 | SNAI3 | 3.15 | 6.33E−03 | 1.34 | LAT | 2.22 | 7.74E−02 | 0.04 |
| TMEM54 | 7.34 | 2.66E−12 | 1.31 | ENTPD8 | 4.57 | 2.83E−05 | 1.47 | MIF4GD | 3.15 | 6.33E−03 | 1.12 | DHX36 | 2.22 | 7.74E−02 | 0.03 |
| NDST1 | 7.34 | 2.64E−12 | 0.9 | PPP1R1B | 4.57 | 2.85E−05 | 1.07 | NBAT1 | 3.15 | 6.26E−03 | 1.07 | FAM98C | 2.22 | 7.69E−02 | 0.02 |
| NCMAP | 7.34 | 2.63E−12 | 0.72 | SEMA3E | 4.57 | 2.88E−05 | 0.82 | RUNX1T1 | 3.15 | 6.28E−03 | 0.69 | RDX | 2.22 | 7.69E−02 | −0.02 |
| PDIA5 | 7.34 | 2.68E−12 | 0.53 | ISCU | 4.57 | 2.83E−05 | 0.69 | SGIP1 | 3.15 | 6.30E−03 | 0.67 | CREBL2 | 2.22 | 7.72E−02 | −0.03 |
| KCTD20 | 7.34 | 2.62E−12 | 0.39 | AMIGO2 | 4.57 | 2.80E−05 | 0.67 | TNFSF15 | 3.15 | 6.35E−03 | 0.66 | JPH1 | 2.22 | 7.71E−02 | −0.04 |
| IL20RA | 7.33 | 2.88E−12 | 0.69 | SPTY2D1 | 4.57 | 2.82E−05 | 0.36 | DUSP5 | 3.15 | 6.29E−03 | 0.28 | RFX1 | 2.22 | 7.80E−02 | −0.16 |
| ATF4 | 7.33 | 2.85E−12 | 0.43 | PKIG | 4.57 | 2.91E−05 | 0.33 | EXOSC6 | 3.15 | 6.40E−03 | 0.2 | GNG4 | 2.21 | 7.85E−02 | 1.33 |
| IFIH1 | 7.31 | 3.15E−12 | 0.74 | ATP6V0B | 4.57 | 2.86E−05 | 0.26 | DLAT | 3.15 | 6.34E−03 | 0.14 | IL12A-AS1 | 2.21 | 7.88E−02 | 1.25 |
| RNF181 | 7.3 | 3.46E−12 | 1.99 | HMG20A | 4.57 | 2.88E−05 | 0.19 | SSX2IP | 3.15 | 6.34E−03 | 0.14 | KLF8 | 2.21 | 7.96E−02 | 0.84 |
| CENPB | 7.3 | 3.45E−12 | 1.36 | SEC24A | 4.57 | 2.82E−05 | 0.17 | SLU7 | 3.15 | 6.35E−03 | 0.11 | AP5S1 | 2.21 | 7.96E−02 | 0.76 |
| PTRH1 | 7.3 | 3.53E−12 | 1.17 | ITGA9 | 4.56 | 2.94E−05 | 0.82 | FRAS1 | 3.15 | 6.26E−03 | 0.07 | MGAT2 | 2.21 | 7.96E−02 | 0.53 |
| TP53BP1 | 7.3 | 3.42E−12 | 0.35 | MYOM3 | 4.56 | 2.95E−05 | 0.6 | GTF2B | 3.15 | 6.28E−03 | −0.02 | DCLK2 | 2.21 | 7.94E−02 | 0.5 |
| ATP6AP1 | 7.29 | 3.86E−12 | 1.01 | YIF1B | 4.56 | 2.96E−05 | 0.57 | AC005281.1 | 3.14 | 6.55E−03 | 3.07 | MTHFS | 2.21 | 7.83E−02 | 0.36 |
| ATG4B | 7.29 | 3.66E−12 | 0.32 | PARP16 | 4.56 | 3.02E−05 | 0.5 | ITGA4 | 3.14 | 6.64E−03 | 1.5 | CEP131 | 2.21 | 7.85E−02 | 0.27 |
| CLTCL1 | 7.28 | 4.17E−12 | 1.09 | AC026167.1 | 4.56 | 3.02E−05 | 0.35 | BRICD5 | 3.14 | 6.47E−03 | 0.75 | MVK | 2.21 | 7.92E−02 | 0.21 |
| CXXC5 | 7.28 | 4.08E−12 | 0.55 | BAX | 4.56 | 2.96E−05 | 0.3 | ALKBH7 | 3.14 | 6.65E−03 | 0.7 | SC5D | 2.21 | 7.96E−02 | 0.12 |
| IFT74 | 7.28 | 3.97E−12 | 0.53 | PARP15 | 4.55 | 3.15E−05 | 2.32 | MEIS2 | 3.14 | 6.65E−03 | 0.15 | CREBBP | 2.21 | 7.97E−02 | 0.1 |
| ARF1 | 7.28 | 4.06E−12 | 0.36 | TMEM11 | 4.55 | 3.12E−05 | 1.02 | FEM1C | 3.14 | 6.50E−03 | 0.12 | RPN2 | 2.21 | 7.89E−02 | 0.05 |
| GLYR1 | 7.28 | 3.98E−12 | 0.33 | SLC38A7 | 4.55 | 3.14E−05 | 0.68 | PTPRU | 3.14 | 6.54E−03 | 0.12 | BNIP3L | 2.21 | 7.91E−02 | 0.02 |
| DDRGK1 | 7.27 | 4.35E−12 | 0.98 | CBR3 | 4.55 | 3.17E−05 | 0.59 | ITFG1 | 3.14 | 6.66E−03 | 0.1 | MTMR2 | 2.21 | 7.96E−02 | −0.01 |
| MRPL55 | 7.27 | 4.21E−12 | 0.8 | SNRPB | 4.55 | 3.18E−05 | 0.49 | UGDH-AS1 | 3.14 | 6.58E−03 | 0.04 | SRGAP2B | 2.21 | 7.82E−02 | −0.04 |
| SPSB1 | 7.27 | 4.23E−12 | 0.66 | FKBP14 | 4.55 | 3.11E−05 | 0.31 | MED24 | 3.14 | 6.60E−03 | 0.03 | LONP1 | 2.21 | 7.98E−02 | −0.13 |
| DZIP3 | 7.27 | 4.24E−12 | 0.57 | TTC39A | 4.55 | 3.13E−05 | 0.28 | MSL1 | 3.14 | 6.65E−03 | 0.02 | PGM3 | 2.21 | 7.97E−02 | −0.2 |
| NUTF2 | 7.26 | 4.69E−12 | 0.7 | CSNK2A1 | 4.55 | 3.07E−05 | 0.21 | TP53RK | 3.13 | 6.71E−03 | 1.13 | ZNF730 | 2.2 | 8.14E−02 | 1.69 |
| C17orf67 | 7.26 | 4.57E−12 | 0.59 | ERC1 | 4.55 | 3.11E−05 | 0.18 | MAPK8IP1 | 3.13 | 6.81E−03 | 1.1 | LAMP3 | 2.2 | 8.05E−02 | 1.36 |
| CARHSP1 | 7.26 | 4.54E−12 | 0.42 | SPATA6 | 4.55 | 3.14E−05 | 0.17 | ALDH1L2 | 3.13 | 6.72E−03 | 0.8 | ERICH6 | 2.2 | 8.15E−02 | 1.11 |
| NRXN3 | 7.25 | 5.11E−12 | 1.01 | MTOR | 4.55 | 3.16E−05 | 0.15 | PMP22 | 3.13 | 6.79E−03 | 0.46 | FER1L5 | 2.2 | 8.08E−02 | 1.01 |
| SMIM8 | 7.25 | 5.02E−12 | 0.85 | SNTG1 | 4.54 | 3.26E−05 | 1.08 | TMEM14C | 3.13 | 6.77E−03 | 0.16 | SLC22A1 | 2.2 | 8.03E−02 | 0.93 |
| HIST2H2BE | 7.25 | 5.02E−12 | 0.75 | RWDD1 | 4.54 | 3.23E−05 | 0.56 | PAM | 3.13 | 6.76E−03 | 0.12 | OSGIN1 | 2.2 | 8.02E−02 | 0.74 |
| SPDYE1 | 7.24 | 5.34E−12 | 1.07 | H1FX | 4.54 | 3.22E−05 | 0.42 | MRPL32 | 3.13 | 6.84E−03 | 0.1 | PPP1R42 | 2.2 | 8.16E−02 | 0.73 |
| MAX | 7.24 | 5.48E−12 | 0.33 | TRAM2 | 4.54 | 3.19E−05 | 0.4 | M6PR | 3.13 | 6.73E−03 | 0.07 | UNC5D | 2.2 | 7.99E−02 | 0.44 |
| ZBP1 | 7.23 | 5.68E−12 | 2.37 | ZNF736 | 4.54 | 3.29E−05 | 0.34 | DNAJC17 | 3.13 | 6.68E−03 | 0.06 | GMPPB | 2.2 | 8.16E−02 | 0.37 |
| LOXL1 | 7.23 | 5.96E−12 | 1.03 | SLC25A3 | 4.54 | 3.26E−05 | 0.24 | HACD2 | 3.13 | 6.78E−03 | 0.05 | TP53TG1 | 2.2 | 8.16E−02 | 0.34 |
| NPIPA1 | 7.23 | 5.79E−12 | 0.96 | ASPHD1 | 4.54 | 3.22E−05 | 0.21 | NBN | 3.13 | 6.73E−03 | 0.03 | SDR42E1 | 2.2 | 8.03E−02 | 0.33 |
| SLC25A27 | 7.23 | 5.92E−12 | 0.93 | NANS | 4.54 | 3.25E−05 | 0.19 | NBPF15 | 3.13 | 6.76E−03 | −0.02 | IL18R1 | 2.2 | 8.15E−02 | 0.24 |
| SRP14 | 7.23 | 5.66E−12 | 0.74 | PRKAR1A | 4.54 | 3.27E−05 | 0.16 | DVL1 | 3.13 | 6.76E−03 | −0.02 | KCTD21 | 2.2 | 8.03E−02 | 0.2 |
| DGCR2 | 7.23 | 5.96E−12 | 0.38 | NUS1 | 4.54 | 3.28E−05 | 0.13 | KCNB2 | 3.12 | 6.92E−03 | 1.23 | SEC24B-AS1 | 2.2 | 8.03E−02 | 0.17 |
| PAK3 | 7.22 | 6.30E−12 | 1.76 | TMED7-TICAM2 | 4.53 | 3.44E−05 | 1 | SPSB2 | 3.12 | 6.89E−03 | 0.61 | POP5 | 2.2 | 8.10E−02 | 0.15 |
| WNT7B | 7.22 | 6.06E−12 | 1.27 | WTIP | 4.53 | 3.40E−05 | 0.61 | CASC1 | 3.12 | 6.89E−03 | 0.51 | MTERF2 | 2.2 | 8.16E−02 | 0.13 |
| BHLHE41 | 7.22 | 6.38E−12 | 0.88 | AVIL | 4.53 | 3.38E−05 | 0.33 | UBAP1L | 3.12 | 7.00E−03 | 0.48 | TONSL | 2.2 | 8.15E−02 | 0.07 |
| RNF217 | 7.22 | 6.00E−12 | 0.51 | FBXW5 | 4.53 | 3.41E−05 | 0.19 | TMOD2 | 3.12 | 6.89E−03 | 0.27 | INTS12 | 2.2 | 8.00E−02 | 0.04 |
| C19orf66 | 7.22 | 6.13E−12 | 0.51 | KLF9 | 4.53 | 3.35E−05 | 0.18 | USB1 | 3.12 | 7.05E−03 | 0.2 | BAG5 | 2.2 | 8.06E−02 | −0.02 |
| PTK6 | 7.22 | 6.28E−12 | 0.35 | MRPL50 | 4.52 | 3.55E−05 | 0.74 | GSTO1 | 3.12 | 6.88E−03 | 0.19 | CNIH1 | 2.2 | 8.14E−02 | −0.03 |
| METAP2 | 7.21 | 6.86E−12 | 0.41 | EHMT2 | 4.52 | 3.50E−05 | 0.39 | LRP11 | 3.12 | 6.99E−03 | 0.16 | CCDC47 | 2.2 | 8.03E−02 | −0.08 |
| SPOCK1 | 7.2 | 7.35E−12 | 0.76 | RIC8A | 4.52 | 3.57E−05 | 0.34 | KIF1BP | 3.12 | 7.07E−03 | 0.15 | HNRNPAB | 2.2 | 8.07E−02 | −0.11 |
| CENPT | 7.2 | 7.15E−12 | 0.54 | NVL | 4.52 | 3.60E−05 | 0.19 | GTF3C5 | 3.12 | 6.92E−03 | 0.14 | BMI1 | 2.2 | 8.11E−02 | −0.14 |
| SLC25A43 | 7.19 | 7.64E−12 | 0.4 | ZNF682 | 4.51 | 3.71E−05 | 1.02 | COMMD4 | 3.12 | 7.06E−03 | 0.14 | PDE8B | 2.19 | 8.28E−02 | 2.18 |
| BCAM | 7.18 | 8.12E−12 | 1.16 | MAPKAPK5-AS1 | 4.51 | 3.76E−05 | 0.81 | ETFA | 3.12 | 6.88E−03 | 0.13 | PHF21B | 2.19 | 8.25E−02 | 2.03 |
| CCDC30 | 7.18 | 8.22E−12 | 0.72 | PALM2 | 4.51 | 3.66E−05 | 0.67 | DYNC1LI2 | 3.12 | 6.96E−03 | 0.13 | AC005253.2 | 2.19 | 8.19E−02 | 1.95 |
| MDK | 7.18 | 8.22E−12 | 0.59 | SYNC | 4.51 | 3.77E−05 | 0.61 | SEC14L2 | 3.12 | 7.04E−03 | 0.13 | LRFN3 | 2.19 | 8.24E−02 | 1.16 |
| ULK1 | 7.18 | 8.04E−12 | 0.46 | PRPF19 | 4.51 | 3.72E−05 | 0.59 | GFOD2 | 3.12 | 6.89E−03 | 0.06 | ABCC11 | 2.19 | 8.25E−02 | 1.04 |
| SF3B2 | 7.18 | 8.10E−12 | 0.45 | ZNF675 | 4.51 | 3.75E−05 | 0.38 | NR2F1-AS1 | 3.12 | 6.92E−03 | 0.05 | PXDN | 2.19 | 8.20E−02 | 0.58 |
| CCND2 | 7.17 | 8.82E−12 | 1.1 | TUBG2 | 4.51 | 3.66E−05 | 0.35 | THEM6 | 3.11 | 7.09E−03 | 0.82 | DPH2 | 2.19 | 8.31E−02 | 0.5 |
| NDUFB9 | 7.17 | 8.93E−12 | 0.85 | TIMP1 | 4.51 | 3.77E−05 | 0.33 | CCDC189 | 3.11 | 7.17E−03 | 0.64 | LINC01250 | 2.19 | 8.24E−02 | 0.42 |
| MAP1LC3A | 7.17 | 8.55E−12 | 0.52 | ARHGAP31 | 4.51 | 3.72E−05 | 0.32 | PRICKLE3 | 3.11 | 7.25E−03 | 0.54 | ARNTL2 | 2.19 | 8.27E−02 | 0.12 |
| U2SURP | 7.17 | 8.81E−12 | 0.3 | GRN | 4.51 | 3.79E−05 | 0.25 | CCDC152 | 3.11 | 7.25E−03 | 0.52 | PKN2-AS1 | 2.19 | 8.19E−02 | 0.07 |
| FBXO2 | 7.16 | 9.37E−12 | 3.07 | SDHB | 4.51 | 3.78E−05 | 0.21 | CDK2AP2 | 3.11 | 7.11E−03 | 0.48 | POLB | 2.19 | 8.34E−02 | 0.05 |
| USP5 | 7.16 | 9.72E−12 | 0.84 | UBN1 | 4.51 | 3.67E−05 | 0.17 | FAT4 | 3.11 | 7.21E−03 | 0.47 | SPRYD4 | 2.19 | 8.34E−02 | 0.04 |
| BCAT2 | 7.16 | 9.77E−12 | 0.84 | ANP32A | 4.51 | 3.68E−05 | 0.16 | MRPS18C | 3.11 | 7.13E−03 | 0.17 | LINC00265 | 2.19 | 8.24E−02 | 0.03 |
| PLAA | 7.16 | 9.74E−12 | 0.58 | TBC1D23 | 4.51 | 3.73E−05 | 0.14 | TBX19 | 3.11 | 7.26E−03 | 0.14 | WDR25 | 2.19 | 8.30E−02 | 0.03 |
| PDZK1IP1 | 7.15 | 9.87E−12 | 0.88 | TMEM246 | 4.5 | 3.83E−05 | 0.82 | WDSUB1 | 3.11 | 7.09E−03 | 0.13 | AIFM1 | 2.19 | 8.18E−02 | 0.01 |
| PSMG3 | 7.15 | 1.05E−11 | 0.64 | SULT1A1 | 4.5 | 3.85E−05 | 0.56 | SLC6A20 | 3.11 | 7.16E−03 | 0.12 | DUT | 2.19 | 8.16E−02 | −0.02 |
| ANAPC2 | 7.14 | 1.07E−11 | 1.23 | LRPAP1 | 4.5 | 3.86E−05 | 0.54 | GANC | 3.11 | 7.18E−03 | 0.08 | MRPL24 | 2.19 | 8.23E−02 | −0.02 |
| DRAP1 | 7.14 | 1.08E−11 | 1.13 | PANX1 | 4.5 | 3.94E−05 | 0.44 | CEBPZ | 3.11 | 7.13E−03 | 0.05 | PLCD3 | 2.19 | 8.28E−02 | −0.05 |
| RCAN1 | 7.14 | 1.08E−11 | 1.09 | ZNF701 | 4.5 | 3.87E−05 | 0.31 | RMDN2 | 3.11 | 7.24E−03 | 0.05 | RBM23 | 2.19 | 8.26E−02 | −0.07 |
| NQO1 | 7.14 | 1.12E−11 | 0.5 | ZNF618 | 4.5 | 3.96E−05 | 0.26 | PURB | 3.11 | 7.12E−03 | 0.04 | BRPF3 | 2.19 | 8.23E−02 | −0.09 |
| SNRPC | 7.13 | 1.16E−11 | 1.14 | EPHB4 | 4.5 | 3.82E−05 | 0.23 | PRPF6 | 3.11 | 7.27E−03 | 0.04 | SCRN2 | 2.19 | 8.27E−02 | −0.09 |
| PLEKHH3 | 7.13 | 1.14E−11 | 0.52 | CAMKK2 | 4.5 | 3.92E−05 | 0.17 | RAB18 | 3.11 | 7.13E−03 | 0.01 | CEACAM8 | 2.18 | 8.52E−02 | 2.56 |
| URB1 | 7.13 | 1.18E−11 | 0.46 | DLGAP4 | 4.5 | 3.95E−05 | 0.17 | SF3A3 | 3.11 | 7.17E−03 | −0.01 | NPPA | 2.18 | 8.48E−02 | 1.96 |
| MT-ND3 | 7.13 | 1.16E−11 | 0.38 | SNX24 | 4.5 | 3.90E−05 | 0.15 | PTPN6 | 3.11 | 7.28E−03 | −0.01 | SLC12A3 | 2.18 | 8.36E−02 | 1.17 |
| RPL5 | 7.13 | 1.20E−11 | 0.34 | MMRN2 | 4.49 | 4.00E−05 | 1.44 | TNNT1 | 3.1 | 7.46E−03 | 0.62 | KCNIP3 | 2.18 | 8.52E−02 | 1.13 |
| MMP2 | 7.12 | 1.22E−11 | 1.28 | SSSCA1 | 4.49 | 4.13E−05 | 1.34 | ABRACL | 3.1 | 7.48E−03 | 0.55 | TENM1 | 2.18 | 8.43E−02 | 0.93 |
| CHCHD10 | 7.12 | 1.23E−11 | 1 | C11orf24 | 4.49 | 4.00E−05 | 0.65 | CRKL | 3.1 | 7.48E−03 | 0.16 | GLA | 2.18 | 8.49E−02 | 0.77 |
| SLC22A15 | 7.12 | 1.24E−11 | 0.59 | SETD1B | 4.49 | 4.15E−05 | 0.41 | NAGK | 3.1 | 7.47E−03 | 0.14 | LINC01389 | 2.18 | 8.37E−02 | 0.46 |
| HDAC7 | 7.11 | 1.36E−11 | 0.3 | PRPF31 | 4.49 | 3.99E−05 | 0.29 | ENSA | 3.1 | 7.53E−03 | 0.12 | GPSM1 | 2.18 | 8.36E−02 | 0.2 |
| ADAM12 | 7.1 | 1.44E−11 | 1.02 | TMEM144 | 4.49 | 4.15E−05 | 0.29 | NUP37 | 3.1 | 7.33E−03 | 0.11 | GTF2E1 | 2.18 | 8.42E−02 | 0.16 |
| SLC35G2 | 7.1 | 1.49E−11 | 0.43 | ALOX5 | 4.49 | 4.04E−05 | 0.19 | LAP3 | 3.1 | 7.37E−03 | 0.11 | RIMS1 | 2.18 | 8.40E−02 | 0.12 |
| EMP1 | 7.1 | 1.41E−11 | 0.42 | STK11 | 4.49 | 4.15E−05 | 0.19 | TNRC6B | 3.1 | 7.46E−03 | 0.11 | PEX5 | 2.18 | 8.47E−02 | 0.11 |
| VEPH1 | 7.09 | 1.60E−11 | 1.26 | HK2 | 4.49 | 3.99E−05 | 0.15 | RPF1 | 3.1 | 7.31E−03 | 0.1 | ZNF334 | 2.18 | 8.40E−02 | 0.09 |
| ZNF529 | 7.09 | 1.60E−11 | 0.56 | OPCML | 4.48 | 4.29E−05 | 0.93 | GPR180 | 3.1 | 7.31E−03 | 0.1 | PRDM4 | 2.18 | 8.42E−02 | 0 |
| CBLC | 7.09 | 1.53E−11 | 0.43 | ARMC6 | 4.48 | 4.28E−05 | 0.91 | VPS9D1 | 3.1 | 7.40E−03 | 0.09 | UGGT1 | 2.18 | 8.44E−02 | −0.02 |
| ERGIC2 | 7.09 | 1.53E−11 | 0.35 | SSPN | 4.48 | 4.29E−05 | 0.83 | COL6A1 | 3.1 | 7.43E−03 | 0.09 | DNAJC11 | 2.18 | 8.43E−02 | −0.07 |
| ZNF260 | 7.08 | 1.60E−11 | 0.71 | POLD2 | 4.48 | 4.17E−05 | 0.43 | NDFIP1 | 3.1 | 7.35E−03 | 0.07 | LRRC8B | 2.18 | 8.40E−02 | −0.08 |
| CAPS2 | 7.08 | 1.61E−11 | 0.61 | ACOT8 | 4.48 | 4.31E−05 | 0.38 | TOX4 | 3.1 | 7.35E−03 | 0.03 | TBPL1 | 2.18 | 8.54E−02 | −0.21 |
| TMEM159 | 7.08 | 1.64E−11 | 0.58 | SLC35E2B | 4.48 | 4.23E−05 | 0.34 | BCCIP | 3.1 | 7.46E−03 | 0 | HRASLS5 | 2.17 | 8.71E−02 | 1.65 |
| CLDN11 | 7.07 | 1.76E−11 | 3.67 | BNC2 | 4.48 | 4.21E−05 | 0.25 | TRIM26 | 3.1 | 7.30E−03 | −0.02 | MEI1 | 2.17 | 8.68E−02 | 0.84 |
| AQP5 | 7.07 | 1.83E−11 | 1.06 | FHL2 | 4.48 | 4.28E−05 | 0.24 | ST8SIA6-AS1 | 3.09 | 7.54E−03 | 3.38 | ZNF497 | 2.17 | 8.65E−02 | 0.68 |
| SLC31A1 | 7.07 | 1.83E−11 | 0.76 | ZNF346 | 4.48 | 4.33E−05 | 0.21 | CFAP57 | 3.09 | 7.69E−03 | 1.33 | MED20 | 2.17 | 8.58E−02 | 0.56 |
| DNHD1 | 7.07 | 1.75E−11 | 0.55 | KBTBD12 | 4.48 | 4.26E−05 | 0.2 | BAG2 | 3.09 | 7.57E−03 | 1.1 | NFKBIE | 2.17 | 8.55E−02 | 0.41 |
| MBOAT1 | 7.07 | 1.81E−11 | 0.46 | EEF1A2 | 4.47 | 4.38E−05 | 1.07 | IPO13 | 3.09 | 7.73E−03 | 0.83 | TMEM150C | 2.17 | 8.58E−02 | 0.34 |
| EEF2 | 7.07 | 1.77E−11 | 0.38 | CYB561A3 | 4.47 | 4.51E−05 | 0.76 | SYS1-DBNDD2 | 3.09 | 7.53E−03 | 0.36 | CCDC115 | 2.17 | 8.63E−02 | 0.16 |
| MT-ND2 | 7.06 | 1.92E−11 | 0.51 | ANKRD52 | 4.47 | 4.53E−05 | 0.51 | DNAH12 | 3.09 | 7.64E−03 | 0.29 | RAD51C | 2.17 | 8.62E−02 | 0.15 |
| ENY2 | 7.06 | 1.85E−11 | 0.49 | RNF103-CHMP3 | 4.47 | 4.39E−05 | 0.31 | SRPX2 | 3.09 | 7.55E−03 | 0.22 | PKDCC | 2.17 | 8.58E−02 | 0.03 |
| MED15 | 7.06 | 1.86E−11 | 0.31 | MAPK13 | 4.47 | 4.43E−05 | 0.25 | NRSN2-AS1 | 3.09 | 7.72E−03 | 0.17 | ACAD10 | 2.17 | 8.61E−02 | 0.02 |
| TGFB1 | 7.05 | 2.09E−11 | 0.48 | MFSD12 | 4.47 | 4.46E−05 | 0.23 | LSM14A | 3.09 | 7.62E−03 | 0.14 | IFFO2 | 2.17 | 8.66E−02 | −0.02 |
| PPCDC | 7.05 | 2.10E−11 | 0.39 | NBPF9 | 4.47 | 4.51E−05 | 0.22 | ABCG1 | 3.09 | 7.63E−03 | 0.13 | LINC01301 | 2.17 | 8.59E−02 | −0.09 |
| PPM1K | 7.04 | 2.26E−11 | 1.01 | USP42 | 4.47 | 4.37E−05 | 0.2 | TUBGCP6 | 3.09 | 7.70E−03 | 0.13 | TPPP3 | 2.16 | 8.87E−02 | 1.07 |
| TAOK2 | 7.04 | 2.12E−11 | 0.66 | KLF12 | 4.47 | 4.53E−05 | 0.19 | TTC9C | 3.09 | 7.55E−03 | 0.09 | FUZ | 2.16 | 8.80E−02 | 0.82 |
| SDCBP2-AS1 | 7.04 | 2.26E−11 | 0.66 | TOMM20 | 4.47 | 4.43E−05 | 0.18 | CAMTA1 | 3.09 | 7.59E−03 | 0.08 | RNFT2 | 2.16 | 8.84E−02 | 0.48 |
| SERTAD2 | 7.04 | 2.16E−11 | 0.64 | ATP2A1 | 4.46 | 4.71E−05 | 0.77 | DCTN2 | 3.09 | 7.72E−03 | 0.07 | C12orf10 | 2.16 | 8.77E−02 | 0.47 |
| PLD3 | 7.04 | 2.26E−11 | 0.38 | SESN2 | 4.46 | 4.74E−05 | 0.77 | SLC7A1 | 3.09 | 7.60E−03 | 0.05 | C7 | 2.16 | 8.93E−02 | 0.43 |
| PPARD | 7.04 | 2.23E−11 | 0.35 | NTM | 4.46 | 4.58E−05 | 0.51 | ZFAND1 | 3.09 | 7.70E−03 | 0.05 | LRRC27 | 2.16 | 8.82E−02 | 0.14 |
| RPS19BP1 | 7.03 | 2.41E−11 | 1.18 | ZNF623 | 4.46 | 4.65E−05 | 0.26 | ANKMY2 | 3.09 | 7.74E−03 | 0.04 | SNHG22 | 2.16 | 8.89E−02 | 0.14 |
| CFAP70 | 7.03 | 2.31E−11 | 0.75 | DEPTOR | 4.16 | 4.65E−05 | 0.18 | GORASP2 | 3.09 | 7.55E−03 | 0.02 | SCIN | 2.16 | 8.80E−02 | 0.07 |
| ATP1B3 | 7.03 | 2.29E−11 | 0.25 | KYNU | 4.46 | 4.70E−05 | 0.16 | FAM153B | 3.08 | 7.77E−03 | 2.27 | TOP1MT | 2.16 | 8.92E−02 | 0.06 |
| VTCN1 | 7.02 | 2.50E−11 | 1.66 | IFIT2 | 4.45 | 4.83E−05 | 2.09 | PLEKHF1 | 3.08 | 7.83E−03 | 1.88 | ABR | 2.16 | 8.92E−02 | 0.06 |
| CDR2L | 7.02 | 2.54E−11 | 0.88 | CISD3 | 4.45 | 4.76E−05 | 0.79 | THBS4 | 3.08 | 7.78E−03 | 1.04 | RPUSD4 | 2.16 | 8.93E−02 | 0.06 |
| CES2 | 7.02 | 2.61E−11 | 0.59 | FBXL7 | 4.45 | 4.90E−05 | 0.61 | PALM2-AKAP2 | 3.08 | 7.85E−03 | 0.9 | DDX6 | 2.16 | 8.78E−02 | 0.04 |
| TSC22D4 | 7.02 | 2.54E−11 | 0.56 | PIK3IP1-AS1 | 4.45 | 4.83E−05 | 0.5 | TMED1 | 3.08 | 7.80E−03 | 0.84 | CALCRL | 2.16 | 8.89E−02 | 0 |
| RPL29 | 7.01 | 2.62E−11 | 0.47 | FAM83F | 4.45 | 4.81E−05 | 0.48 | RPL18A | 3.08 | 7.85E−03 | 0.68 | SKI | 2.16 | 8.80E−02 | −0.01 |
| TACSTD2 | 7 | 2.99E−11 | 0.55 | H1F0 | 4.45 | 4.89E−05 | 0.35 | GSK3A | 3.08 | 7.98E−03 | 0.48 | AVEN | 2.16 | 8.87E−02 | −0.01 |
| HM13 | 6.99 | 3.08E−11 | 0.33 | GTF2IRD2 | 4.45 | 4.93E−05 | 0.3 | ASB16-AS1 | 3.08 | 7.83E−03 | 0.39 | CHEK2 | 2.16 | 8.79E−02 | −0.03 |
| IFIT1 | 6.98 | 3.25E−11 | 2.71 | PARP11 | 4.45 | 4.91E−05 | 0.29 | RFK | 3.08 | 7.89E−03 | 0.37 | POLR3C | 2.16 | 8.87E−02 | −0.04 |
| RAN | 6.98 | 3.35E−11 | 0.82 | DTX2 | 4.45 | 4.81E−05 | 0.22 | SLC25A17 | 3.08 | 7.77E−03 | 0.08 | ENTPD7 | 2.16 | 8.87E−02 | −0.04 |
| TMEM41A | 6.98 | 3.37E−11 | 0.45 | C2CD2 | 4.45 | 4.88E−05 | 0.2 | TP5313 | 3.08 | 7.87E−03 | −0.01 | RSBN1 | 2.16 | 8.89E−02 | −0.07 |
| MAATS1 | 6.97 | 3.70E−11 | 1.07 | SIL1 | 4.45 | 4.79E−05 | 0.19 | CDV3 | 3.08 | 7.85E−03 | −0.02 | CUTC | 2.16 | 8.77E−02 | −0.09 |
| PTMS | 6.97 | 3.65E−11 | 0.94 | SSU72 | 4.45 | 4.93E−05 | 0.19 | MEAF6 | 3.08 | 7.82E−03 | −0.05 | TDP2 | 2.16 | 8.78E−02 | −0.14 |
| HELZ2 | 6.97 | 3.57E−11 | 0.64 | G3BP2 | 4.45 | 4.93E−05 | 0.11 | ZCWPW2 | 3.07 | 8.17E−03 | 2.74 | MGAT5B | 2.15 | 8.95E−02 | 1.23 |
| GCN1 | 6.97 | 3.65E−11 | 0.4 | APOBEC2 | 4.44 | 5.16E−05 | 2.31 | SPATC1 | 3.07 | 8.15E−03 | 1.14 | CUBN | 2.15 | 9.04E−02 | 0.36 |
| FAM184B | 6.96 | 3.71E−11 | 0.99 | LARP6 | 4.44 | 5.02E−05 | 1.22 | NOXO1 | 3.07 | 8.04E−03 | 0.92 | BTN2A2 | 2.15 | 8.96E−02 | 0.35 |
| FOXRED1 | 6.96 | 3.81E−11 | 0.77 | PYCARD | 4.44 | 5.13E−05 | 1.05 | MRPL49 | 3.07 | 8.14E−03 | 0.89 | C1orf109 | 2.15 | 9.02E−02 | 0.34 |
| CEP89 | 6.96 | 3.88E−11 | 0.43 | ADCY2 | 4.44 | 5.03E−05 | 0.7 | SMOC2 | 3.07 | 8.13E−03 | 0.73 | CCDC127 | 2.15 | 8.95E−02 | 0.32 |
| RAB40C | 6.96 | 3.85E−11 | 0.33 | MTHFD2 | 4.44 | 4.98E−05 | 0.52 | NLRX1 | 3.07 | 8.03E−03 | 0.31 | SLC25A10 | 2.15 | 9.12E−02 | 0.32 |
| PLXNB2 | 6.96 | 3.95E−11 | 0.33 | ZMAT1 | 4.44 | 5.12E−05 | 0.47 | MCPH1-AS1 | 3.07 | 8.08E−03 | 0.26 | MRPL46 | 2.15 | 8.94E−02 | 0.24 |
| SLC14A1 | 6.95 | 4.01E−11 | 2.64 | IFT52 | 4.44 | 5.06E−05 | 0.25 | ERMAP | 3.07 | 8.15E−03 | 0.19 | GLMP | 2.15 | 9.06E−02 | 0.17 |
| GABRB3 | 6.95 | 3.96E−11 | 1.12 | COPS7B | 4.44 | 4.99E−05 | 0.24 | ACYP2 | 3.07 | 8.01E−03 | 0.13 | IL18 | 2.15 | 9.10E−02 | 0.04 |
| PTCHD1-AS | 6.95 | 4.01E−11 | 1.12 | TFEB | 4.44 | 5.14E−05 | 0.17 | FKBP4 | 3.07 | 8.21E−03 | 0.12 | MCM8 | 2.15 | 9.01E−02 | 0.03 |
| DNAH7 | 6.95 | 4.08E−11 | 0.45 | IQCG | 4.44 | 5.02E−05 | 0.15 | FURIN | 3.07 | 8.21E−03 | 0.06 | CDKL5 | 2.15 | 9.06E−02 | 0.02 |
| S100A10 | 6.95 | 4.20E−11 | 0.34 | COL24A1 | 4.43 | 5.39E−05 | 1.22 | SLC35C2 | 3.07 | 8.07E−03 | 0.02 | HCFC2 | 2.15 | 9.06E−02 | 0.01 |
| PPP1R7 | 6.94 | 4.33E−11 | 0.45 | SPARCL1 | 4.43 | 5.34E−05 | 0.64 | PARP6 | 3.07 | 8.09E−03 | 0 | ANKRD13D | 2.15 | 9.02E−02 | 0 |
| RAB1B | 6.93 | 4.68E−11 | 0.8 | ZNF703 | 4.43 | 5.19E−05 | 0.62 | YTHDF1 | 3.07 | 8.02E−03 | −0.01 | PLCG1 | 2.15 | 9.06E−02 | −0.04 |
| APRT | 6.93 | 4.77E−11 | 0.6 | ANKRD29 | 4.43 | 5.23E−05 | 0.46 | AGAP6 | 3.07 | 8.22E−03 | −0.02 | BICD2 | 2.15 | 9.00E−02 | −0.09 |
| AZIN1-AS1 | 6.93 | 4.76E−11 | 0.49 | AMDHD2 | 4.43 | 5.37E−05 | 0.45 | PSCA | 3.07 | 8.13E−03 | −0.33 | TMED5 | 2.15 | 9.12E−02 | −0.13 |
| KDM2B | 6.93 | 4.68E−11 | 0.44 | GCA | 4.43 | 5.20E−05 | 0.22 | RABIF | 3.06 | 8.41E−03 | 0.72 | HSPB8 | 2.14 | 9.24E−02 | 1.51 |
| DGKZ | 6.93 | 4.61E−11 | 0.37 | PARN | 4.43 | 5.20E−05 | 0.2 | NR2C2AP | 3.06 | 8.29E−03 | 0.71 | C9orf116 | 2.14 | 9.30E−02 | 1.26 |
| KCTD10 | 6.92 | 5.18E−11 | 0.86 | NDUFC1 | 4.42 | 5.46E−05 | 0.63 | PMAIP1 | 3.06 | 8.44E−03 | 0.58 | MMP13 | 2.14 | 9.32E−02 | 0.92 |
| ENDOD1 | 6.92 | 4.95E−11 | 0.81 | DNAH6 | 4.42 | 5.52E−05 | 0.6 | SNAPC5 | 3.06 | 8.47E−03 | 0.54 | NCR3LG1 | 2.14 | 9.22E−02 | 0.14 |
| COMMD2 | 6.92 | 4.89E−11 | 0.72 | ITGAE | 4.42 | 5.58E−05 | 0.51 | MTFR1L | 3.06 | 8.32E−03 | 0.38 | TEX14 | 2.14 | 9.31E−02 | 0.07 |
| HOXB7 | 6.91 | 5.29E−11 | 1.66 | LRMP | 4.42 | 5.62E−05 | 0.48 | PRCP | 3.06 | 8.43E−03 | 0.3 | AFTPH | 2.14 | 9.35E−02 | 0.04 |
| C4orf3 | 6.91 | 5.35E−11 | 0.72 | PNPLA6 | 4.42 | 5.46E−05 | 0.24 | UBOX5-AS1 | 3.06 | 8.41E−03 | 0.29 | RPL9 | 2.14 | 9.25E−02 | −0.03 |
| MAP3K14 | 6.91 | 5.47E−11 | 0.43 | NDRG3 | 4.42 | 5.52E−05 | 0.23 | ALDH1A3 | 3.06 | 8.47E−03 | 0.29 | DPH7 | 2.14 | 9.26E−02 | −0.08 |
| TCEA2 | 6.9 | 5.83E−11 | 1.05 | LMF2 | 4.42 | 5.41E−05 | 0.22 | NFU1 | 3.06 | 8.41E−03 | 0.15 | TRIM37 | 2.14 | 9.34E−02 | −0.08 |
| TRAF7 | 6.9 | 5.68E−11 | 0.64 | FZR1 | 4.42 | 5.56E−05 | 0.21 | DUSP10 | 3.06 | 8.28E−03 | 0.12 | ZSWIM3 | 2.14 | 9.24E−02 | −0.11 |
| EVPL | 6.9 | 5.88E−11 | 0.59 | SEC11A | 4.42 | 5.61E−05 | 0.17 | LAMB3 | 3.06 | 8.35E−03 | 0.12 | MMADHC | 2.14 | 9.24E−02 | −0.15 |
| MUC12 | 6.89 | 6.35E−11 | 1.04 | ZBTB43 | 4.42 | 5.54E−05 | 0.15 | SH3BGRL | 3.06 | 8.41E−03 | 0.1 | LRRIQ4 | 2.13 | 9.42E−02 | 1.78 |
| SPON2 | 6.89 | 6.21E−11 | 0.57 | DMD | 4.42 | 5.52E−05 | −0.05 | WDR5 | 3.06 | 8.48E−03 | 0.1 | ZNF114 | 2.13 | 9.40E−02 | 1.64 |
| MAP2K3 | 6.89 | 6.02E−11 | 0.42 | SLC35B2 | 4.41 | 5.79E−05 | 1.25 | UBE2Z | 3.06 | 8.30E−03 | 0.09 | RRS1-AS1 | 2.13 | 9.36E−02 | 1.34 |
| ARRDC1 | 6.89 | 6.29E−11 | 0.35 | GRID2 | 4.41 | 5.70E−05 | 1.19 | CCDC68 | 3.06 | 8.38E−03 | 0.09 | PLK5 | 2.13 | 9.44E−02 | 0.94 |
| MANBA | 6.89 | 6.09E−11 | 0.33 | STXBP5-AS1 | 4.41 | 5.76E−05 | 0.69 | METTL5 | 3.06 | 8.26E−03 | 0.06 | MEPCE | 2.13 | 9.35E−02 | 0.29 |
| AMOTL1 | 6.88 | 6.79E−11 | 0.61 | ETFB | 4.41 | 5.72E−05 | 0.55 | ZNF431 | 3.06 | 8.45E−03 | −0.02 | WDR83 | 2.13 | 9.35E−02 | 0.2 |
| SNHG8 | 6.88 | 6.38E−11 | 0.44 | DOCK3 | 4.41 | 5.73E−05 | 0.46 | DHTKD1 | 3.06 | 8.25E−03 | −0.04 | ENOSF1 | 2.13 | 9.35E−02 | 0.11 |
| CYBA | 6.88 | 6.39E−11 | 0.41 | APIS1 | 4.41 | 5.81E−05 | 0.42 | PCSK2 | 3.05 | 8.74E−03 | 1.8 | APTX | 2.13 | 9.36E−02 | 0 |
| C1orf122 | 6.87 | 6.90E−11 | 1.06 | NARS | 4.41 | 5.81E−05 | 0.41 | PTRHD1 | 3.05 | 8.55E−03 | 1.69 | SLC45A4 | 2.13 | 9.46E−02 | −0.01 |
| SNRPD3 | 6.87 | 6.90E−11 | 0.75 | MAP9 | 4.41 | 5.69E−05 | 0.27 | CLDN3 | 3.05 | 8.58E−03 | 1.36 | SAMD9 | 2.13 | 9.37E−02 | −0.03 |
| NAA60 | 6.87 | 6.93E−11 | 0.7 | PTPDC1 | 4.41 | 5.76E−05 | 0.2 | LINC01411 | 3.05 | 8.69E−03 | 1.11 | SPCS1 | 2.13 | 9.36E−02 | −0.04 |
| RMDN3 | 6.87 | 7.06E−11 | 0.57 | TGOLN2 | 4.41 | 5.64E−05 | 0.19 | SLC6A9 | 3.05 | 8.60E−03 | 0.7 | CLDND1 | 2.13 | 9.38E−02 | −0.08 |
| H6PD | 6.87 | 7.21E−11 | 0.57 | CAPN12 | 4.41 | 5.79E−05 | 0.19 | ATP13A4 | 3.05 | 8.52E−03 | 0.55 | FRMPD2 | 2.12 | 9.67E−02 | 1.65 |
| GRIK2 | 6.86 | 7.35E−11 | 1.29 | SARAF | 4.41 | 5.64E−05 | 0.17 | PAF1 | 3.05 | 8.64E−03 | 0.44 | AC009264.1 | 2.12 | 9.56E−02 | 1.09 |
| PDIA4 | 6.86 | 7.61E−11 | 0.63 | STX2 | 4.41 | 5.71E−05 | 0.16 | ADAM22 | 3.05 | 8.58E−03 | 0.43 | GREB1 | 2.12 | 9.74E−02 | 1.09 |
| DMPK | 6.85 | 7.96E−11 | 0.57 | ASIC2 | 4.4 | 6.05E−05 | 1.23 | ZMIZ1-AS1 | 3.05 | 8.74E−03 | 0.27 | OSR2 | 2.12 | 9.56E−02 | 0.98 |
| OTUB1 | 6.85 | 8.05E−11 | 0.54 | DHX37 | 4.4 | 6.06E−05 | 0.48 | CMBL | 3.05 | 8.51E−03 | 0.25 | LINC01277 | 2.12 | 9.56E−02 | 0.76 |
| CUEDC1 | 6.85 | 8.15E−11 | 0.34 | PIK3R4 | 4.4 | 5.94E−05 | 0.21 | ANKRD23 | 3.05 | 8.73E−03 | 0.13 | TCEAL3 | 2.12 | 9.58E−02 | 0.4 |
| LINC01060 | 6.84 | 8.89E−11 | 1.63 | EXPH5 | 4.4 | 6.05E−05 | 0.18 | GPATCH1 | 3.05 | 8.69E−03 | 0.07 | MIIP | 2.12 | 9.77E−02 | 0.36 |
| TMEM214 | 6.84 | 8.88E−11 | 0.68 | ZFPM2 | 4.4 | 6.08E−05 | 0.18 | ATG14 | 3.05 | 8.65E−03 | 0.04 | PIGP | 2.12 | 9.71E−02 | 0.16 |
| ZEB1 | 6.84 | 8.81E−11 | 0.41 | CRTC1 | 4.4 | 6.11E−05 | 0.15 | WAC-AS1 | 3.05 | 8.68E−03 | 0.03 | SLC2A9 | 2.12 | 9.66E−02 | 0.13 |
| ARFGAP1 | 6.84 | 8.77E−11 | 0.34 | EFCAB14 | 4.4 | 6.04E−05 | 0.14 | ZNF266 | 3.05 | 8.70E−03 | 0 | C2orf42 | 2.12 | 9.67E−02 | 0.02 |
| PTK7 | 6.83 | 9.26E−11 | 0.54 | IGFBP6 | 4.39 | 6.32E−05 | 0.83 | CLNS1A | 3.05 | 8.58E−03 | −0.01 | PDHX | 2.12 | 9.61E−02 | −0.06 |
| ARMCX3 | 6.83 | 9.11E−11 | 0.53 | HS1BP3 | 4.39 | 6.34E−05 | 0.42 | CMPK2 | 3.04 | 8.82E−03 | 1.66 | CASC11 | 2.11 | 9.95E−02 | 1.67 |
| PDE9A | 6.83 | 9.27E−11 | 0.52 | POMP | 4.39 | 6.27E−05 | 0.22 | UNC13C | 3.04 | 8.79E−03 | 1.65 | C15orf65 | 2.11 | 9.88E−02 | 1.16 |
| SARS | 6.83 | 9.43E−11 | 0.47 | CASP10 | 4.39 | 6.27E−05 | 0.22 | PCDH19 | 3.04 | 8.85E−03 | 1.14 | ZSCAN2 | 2.11 | 9.80E−02 | 0.52 |
| HPS4 | 6.83 | 9.13E−11 | 0.34 | DCUN1D5 | 4.39 | 6.22E−05 | 0.16 | KLC3 | 3.04 | 8.75E−03 | 0.97 | HMCN1 | 2.11 | 9.97E−02 | 0.31 |
| SLC16A3 | 6.83 | 9.38E−11 | 0.32 | KLF6 | 4.39 | 6.32E−05 | 0.13 | COMMD9 | 3.04 | 8.80E−03 | 0.63 | RERGL | 2.11 | 9.79E−02 | 0.28 |
| SPRY1 | 6.82 | 9.68E−11 | 1.03 | RBM42 | 4.38 | 6.66E−05 | 0.89 | PLTP | 3.04 | 8.87E−03 | 0.57 | MYLIP | 2.11 | 9.93E−02 | 0.28 |
| DAB1 | 6.82 | 1.00E−10 | 0.42 | GTF2F1 | 4.38 | 6.69E−05 | 0.63 | NAALADL2-AS2 | 3.04 | 8.86E−03 | 0.51 | AKTIP | 2.11 | 9.80E−02 | 0.27 |
| WDR1 | 6.81 | 1.07E−10 | 0.37 | OTUD6B-AS1 | 4.38 | 6.48E−05 | 0.28 | RGPD8 | 3.04 | 8.79E−03 | 0.47 | MPDU1 | 2.11 | 9.85E−02 | 0.18 |
| MAVS | 6.81 | 1.05E−10 | 0.35 | PRKCE | 4.38 | 6.56E−05 | 0.2 | ICAM1 | 3.04 | 8.92E−03 | 0.34 | CDH17 | 2.11 | 9.93E−02 | 0.16 |
| TCTN1 | 6.8 | 1.15E−10 | 1.16 | THUMPD3-AS1 | 4.38 | 6.64E−05 | 0.16 | URGCP | 3.04 | 8.88E−03 | 0.23 | ZNF219 | 2.11 | 9.78E−02 | 0.13 |
| SLC52A2 | 6.8 | 1.09E−10 | 0.83 | PCNP | 4.38 | 6.61E−05 | 0.15 | DNM1 | 3.04 | 8.85E−03 | 0.14 | MTX1 | 2.11 | 9.91E−02 | 0.05 |
| EIF5A | 6.79 | 1.20E−10 | 0.9 | HSD17B14 | 4.37 | 6.93E−05 | 1.28 | ENO1 | 3.04 | 8.99E−03 | 0.11 | DNAJC19 | 2.11 | 9.95E−02 | 0.05 |
| FHAD1 | 6.79 | 1.22E−10 | 0.77 | NDUFB11 | 4.37 | 6.92E−05 | 0.92 | MAN2B2 | 3.04 | 8.82E−03 | 0 | PRDM5 | 2.11 | 1.00E−01 | 0.03 |
| CD276 | 6.79 | 1.23E−10 | 0.71 | DDOST | 4.37 | 6.85E−05 | 0.54 | CNN1 | 3.03 | 9.09E−03 | 1.98 | ZCCHC8 | 2.11 | 9.85E−02 | −0.01 |
| ORAOV1 | 6.79 | 1.24E−10 | 0.53 | NPHP1 | 4.37 | 6.86E−05 | 0.36 | NXF3 | 3.03 | 9.10E−03 | 1.49 | TENM3 | 2.11 | 1.00E−01 | −0.03 |
| PKP2 | 6.79 | 1.22E−10 | 0.33 | TXLNA | 4.37 | 6.95E−05 | 0.34 | KIAA1324 | 3.03 | 9.01E−03 | 0.8 | GAS8 | 2.11 | 9.98E−02 | −0.09 |
| CDA | 6.78 | 1.27E−10 | 0.91 | MANBAL | 4.37 | 6.81E−05 | 0.23 | CHRM5 | 3.03 | 9.14E−03 | 0.74 | FAM229B | 2.1 | 1.01E−01 | 1.49 |
| PPP1CA | 6.78 | 1.27E−10 | 0.68 | TRIM47 | 4.37 | 6.95E−05 | 0.21 | METTL7A | 3.03 | 9.23E−03 | 0.51 | HIST1H2BG | 2.1 | 1.00E−01 | 0.91 |
| PPM1G | 6.78 | 1.27E−10 | 0.41 | HBG2 | 4.36 | 7.24E−05 | 3.56 | CTSO | 3.03 | 9.09E−03 | 0.49 | EID2 | 2.1 | 1.01E−01 | 0.86 |
| WDR73 | 6.78 | 1.28E−10 | 0.36 | CHCHD5 | 4.36 | 7.08E−05 | 1.24 | VAV1 | 3.03 | 9.24E−03 | 0.3 | C6orf226 | 2.1 | 1.02E−01 | 0.82 |
| CARS | 6.78 | 1.32E−10 | 0.33 | ZDHHC12 | 4.36 | 7.18E−05 | 1.09 | TCEANC2 | 3.03 | 9.17E−03 | 0.25 | COLEC10 | 2.1 | 1.01E−01 | 0.69 |
| C6orf106 | 6.78 | 1.31E−10 | 0.29 | KLC2 | 4.36 | 7.19E−05 | 0.72 | PBLD | 3.03 | 9.12E−03 | 0.18 | RNF165 | 2.1 | 1.02E−01 | 0.66 |
| COA3 | 6.77 | 1.40E−10 | 1.58 | HS6ST1 | 4.36 | 7.15E−05 | 0.47 | UTP14C | 3.03 | 9.20E−03 | 0.18 | SLC38A4 | 2.1 | 1.00E−01 | 0.64 |
| ISOC2 | 6.77 | 1.41E−10 | 1.38 | C16orf70 | 4.36 | 7.07E−05 | 0.33 | TRIM28 | 3.03 | 9.05E−03 | 0.16 | POLR3K | 2.1 | 1.02E−01 | 0.64 |
| NDUFA1 | 6.77 | 1.41E−10 | 1.27 | NF2 | 4.36 | 7.07E−05 | 0.23 | HACD3 | 3.03 | 9.12E−03 | 0.06 | DEPDC4 | 2.1 | 1.02E−01 | 0.57 |
| DDX54 | 6.77 | 1.35E−10 | 0.98 | C1orf159 | 4.36 | 7.22E−05 | 0.22 | METTL2B | 3.03 | 9.24E−03 | 0.06 | NBPF20 | 2.1 | 1.02E−01 | 0.5 |
| SORT1 | 6.77 | 1.36E−10 | 0.49 | ST13 | 4.36 | 7.16E−05 | 0.21 | GJA1 | 3.02 | 9.47E−03 | 1.19 | ESR1 | 2.1 | 1.02E−01 | 0.45 |
| VAC14 | 6.77 | 1.38E−10 | 0.39 | PNPT1 | 4.36 | 7.04E−05 | 0.16 | FRMPD4 | 3.02 | 9.33E−03 | 0.93 | SPDEF | 2.1 | 1.00E−01 | 0.39 |
| PLA2G4C | 6.76 | 1.52E−10 | 1.13 | 10-Mar | 4.35 | 7.36E−05 | 1.65 | SMDT1 | 3.02 | 9.44E−03 | 0.73 | TP53TG5 | 2.1 | 1.00E−01 | 0.21 |
| IGF2BP2-AS1 | 6.76 | 1.52E−10 | 0.82 | CDC42EP2 | 4.35 | 7.45E−05 | 1.38 | G6PD | 3.02 | 9.48E−03 | 0.65 | IGHMBP2 | 2.1 | 1.02E−01 | 0.09 |
| TOMM40 | 6.76 | 1.48E−10 | 0.77 | FUK | 4.35 | 7.53E−05 | 0.32 | NR1D1 | 3.02 | 9.51E−03 | 0.48 | CAMK2N1 | 2.1 | 1.02E−01 | 0.03 |
| SCYL1 | 6.76 | 1.46E−10 | 0.73 | SLC15A4 | 4.35 | 7.59E−05 | 0.24 | CREB3L1 | 3.02 | 9.39E−03 | −0.03 | MIS12 | 2.1 | 1.01E−01 | −0.03 |
| OAT | 6.76 | 1.44E−10 | 0.64 | MAFK | 4.35 | 7.42E−05 | 0.21 | ZFP82 | 3.01 | 9.80E−03 | 1.53 | ADCK1 | 2.1 | 1.01E−01 | −0.06 |
| EXOC3 | 6.76 | 1.44E−10 | 0.5 | SIGIRR | 4.35 | 7.60E−05 | 0.21 | FBP2 | 3.01 | 9.72E−03 | 1.46 | FAM53C | 2.1 | 1.00E−01 | −0.1 |
| ABCC4 | 6.75 | 1.54E−10 | 0.56 | ASAP1 | 4.35 | 7.47E−05 | 0.17 | PCP4 | 3.01 | 9.78E−03 | 1.18 | EML3 | 2.1 | 1.02E−01 | −0.1 |
| NOP10 | 6.74 | 1.69E−10 | 1.02 | STAG1 | 4.35 | 7.58E−05 | 0.14 | IFIT5 | 3.01 | 9.67E−03 | 1.13 | ELDR | 2.09 | 1.03E−01 | 1.19 |
| LGALS9 | 6.74 | 1.65E−10 | 0.64 | TRIB3 | 4.34 | 7.75E−05 | 1.2 | FAM110A | 3.01 | 9.62E−03 | 0.48 | ZNF286B | 2.09 | 1.03E−01 | 1.16 |
| ATP10D | 6.74 | 1.67E−10 | 0.46 | ZNF629 | 4.34 | 7.92E−05 | 1.14 | SLC25A1 | 3.01 | 9.61E−03 | 0.28 | CCDC69 | 2.09 | 1.02E−01 | 1.08 |
| ELL | 6.74 | 1.71E−10 | 0.4 | ALG3 | 4.34 | 7.70E−05 | 0.64 | ARMC3 | 3.01 | 9.77E−03 | 0.26 | UNC13A | 2.09 | 1.03E−01 | 1.05 |
| CDK18 | 6.73 | 1.85E−10 | 0.37 | JRKL | 4.34 | 7.71E−05 | 0.45 | ATAD3A | 3.01 | 9.77E−03 | 0.24 | MDC1-AS1 | 2.09 | 1.02E−01 | 1.02 |
| NTF4 | 6.72 | 1.96E−10 | 3.94 | PSTPIP2 | 4.34 | 7.80E−05 | 0.36 | PPM1F | 3.01 | 9.60E−03 | 0.19 | AC005355.1 | 2.09 | 1.04E−01 | 0.73 |
| EIF2B3 | 6.72 | 1.97E−10 | 0.48 | MOCOS | 4.34 | 7.75E−05 | 0.27 | ACTA2 | 3.01 | 9.77E−03 | 0.11 | PRKCQ | 2.09 | 1.03E−01 | 0.7 |
| LCMT1 | 6.72 | 1.95E−10 | 0.36 | WDR34 | 4.34 | 7.81E−05 | 0.26 | HDAC6 | 3.01 | 9.80E−03 | 0.11 | COL5A3 | 2.09 | 1.03E−01 | 0.7 |
| USP36 | 6.72 | 1.87E−10 | 0.33 | LURAPIL | 4.34 | 7.92E−05 | 0.23 | PRELID3B | 3.01 | 9.68E−03 | 0.09 | KIAA1586 | 2.09 | 1.03E−01 | 0.51 |
| NFATC3 | 6.72 | 1.96E−10 | 0.32 | EPCAM | 4.34 | 7.75E−05 | 0.18 | ETHE1 | 3.01 | 9.80E−03 | 0.08 | TCTN2 | 2.09 | 1.03E−01 | 0.42 |
| ATG9A | 6.71 | 1.98E−10 | 2.51 | TXNDC12 | 4.34 | 7.83E−05 | 0.18 | EBPL | 3.01 | 9.80E−03 | 0.04 | BCL2L12 | 2.09 | 1.04E−01 | 0.41 |
| NT5DC3 | 6.71 | 2.06E−10 | 0.62 | C21orf2 | 4.33 | 7.95E−05 | 0.44 | ZNF561 | 3.01 | 9.81E−03 | 0.04 | DHX58 | 2.09 | 1.03E−01 | 0.27 |
| LINC01376 | 6.71 | 2.06E−10 | 0.39 | FCHO1 | 4.33 | 8.26E−05 | 0.44 | P4HA2 | 3.01 | 9.83E−03 | 0.02 | TMEM266 | 2.09 | 1.03E−01 | 0.23 |
| UBALD1 | 6.7 | 2.12E−10 | 0.96 | FAM182B | 4.33 | 8.01E−05 | 0.3 | DDX27 | 3.01 | 9.57E−03 | 0.01 | SYNE4 | 2.09 | 1.03E−01 | 0.17 |
| UFL1-AS1 | 6.7 | 2.16E−10 | 0.72 | POLDIP3 | 4.33 | 8.12E−05 | 0.28 | SMIM24 | 3 | 1.01E−02 | 1.39 | ZNF830 | 2.09 | 1.04E−01 | 0.15 |
| IQCH-AS1 | 6.7 | 2.19E−10 | 0.5 | LINC01473 | 4.33 | 8.28E−05 | 0.22 | FADS2 | 3 | 1.00E−02 | 0.7 | MEGF9 | 2.09 | 1.03E−01 | −0.03 |
| ALDH7A1 | 6.7 | 2.12E−10 | 0.41 | NFIC | 4.33 | 8.12E−05 | 0.17 | BGN | 3 | 9.87E−03 | 0.6 | ME3 | 2.09 | 1.04E−01 | −0.03 |
| TUFM | 6.7 | 2.24E−10 | 0.41 | UGP2 | 4.33 | 8.01E−05 | 0.14 | MED12L | 3 | 9.91E−03 | 0.41 | STK32C | 2.09 | 1.04E−01 | −0.04 |
| CALCOCO2 | 6.7 | 2.13E−10 | 0.29 | C6orf47 | 4.32 | 8.50E−05 | 1.3 | SMIM10L1 | 3 | 9.96E−03 | 0.34 | FAM199X | 2.09 | 1.03E−01 | −0.1 |
| C16orf46 | 6.69 | 2.30E−10 | 3.88 | EML5 | 4.32 | 8.54E−05 | 0.93 | CXorf56 | 3 | 1.01E−02 | 0.31 | ATG3 | 2.09 | 1.04E−01 | −0.11 |
| CC2D1A | 6.69 | 2.32E−10 | 0.33 | TNFAIP1 | 4.32 | 8.59E−05 | 0.28 | NINJ2 | 3 | 1.00E−02 | 0.24 | TSIX | 2.08 | 1.05E−01 | 2.84 |
| MED29 | 6.68 | 2.48E−10 | 1.11 | GALNT6 | 4.32 | 8.32E−05 | 0.27 | LBH | 3 | 1.00E−02 | 0.22 | B4GALNT1 | 2.08 | 1.06E−01 | 2.56 |
| BAD | 6.68 | 2.46E−10 | 0.99 | RIPK2 | 4.32 | 8.46E−05 | 0.17 | AKAP7 | 3 | 1.01E−02 | 0.18 | LINC01340 | 2.08 | 1.05E−01 | 1.13 |
| NACC1 | 6.68 | 2.54E−10 | 0.74 | C1orf50 | 4.31 | 8.89E−05 | 3.15 | C5orf22 | 3 | 1.00E−02 | 0.11 | SRRM2-AS1 | 2.08 | 1.06E−01 | 0.84 |
| GOT2 | 6.68 | 2.46E−10 | 0.62 | NACA2 | 4.31 | 8.94E−05 | 1.17 | WWTR1 | 3 | 9.86E−03 | 0.09 | CHST6 | 2.08 | 1.05E−01 | 0.77 |
| SERPINH1 | 6.67 | 2.65E−10 | 0.77 | COL4A3 | 4.31 | 8.90E−05 | 0.92 | C2orf88 | 3 | 9.87E−03 | 0.08 | MYBL1 | 2.08 | 1.05E−01 | 0.7 |
| PSMC4 | 6.67 | 2.73E−10 | 0.65 | SNRNP35 | 4.31 | 8.77E−05 | 0.35 | CEP57 | 3 | 9.93E−03 | 0.08 | ITGB2-AS1 | 2.08 | 1.06E−01 | 0.59 |
| RBMS3 | 6.67 | 2.59E−10 | 0.47 | SDHA | 4.31 | 8.69E−05 | 0.25 | STK39 | 3 | 1.01E−02 | 0.05 | DLG4 | 2.08 | 1.05E−01 | 0.54 |
| DHRS3 | 6.67 | 2.59E−10 | 0.33 | OAF | 4.31 | 8.78E−05 | 0.23 | SIAE | 3 | 1.01E−02 | 0.04 | ADGRG5 | 2.08 | 1.06E−01 | 0.54 |
| TSPYL2 | 6.66 | 2.88E−10 | 0.91 | TTC38 | 4.31 | 8.82E−05 | 0.23 | BECN1 | 3 | 9.93E−03 | 0.02 | ZNF805 | 2.08 | 1.06E−01 | 0.53 |
| ZNF7 | 6.66 | 2.89E−10 | 0.49 | PDAP1 | 4.31 | 8.89E−05 | 0.23 | USP6 | 2.99 | 1.04E−02 | 1.24 | RGMB | 2.08 | 1.05E−01 | 0.3 |
| C16orf58 | 6.65 | 3.08E−10 | 0.64 | SHROOM1 | 4.31 | 8.92E−05 | 0.21 | PPFIA3 | 2.99 | 1.04E−02 | 0.82 | CARD14 | 2.08 | 1.05E−01 | 0.23 |
| ITFG2 | 6.65 | 3.09E−10 | 0.32 | STX4 | 4.31 | 8.78E−05 | 0.18 | TRPT1 | 2.99 | 1.02E−02 | 0.54 | PDRG1 | 2.08 | 1.05E−01 | 0.15 |
| TMCC3 | 6.64 | 3.28E−10 | 0.51 | MTDH | 4.31 | 8.88E−05 | 0.17 | C2CD2L | 2.99 | 1.03E−02 | 0.41 | NIF3L1 | 2.08 | 1.05E−01 | 0.15 |
| CAPN15 | 6.64 | 3.17E−10 | 0.45 | SEC63 | 4.31 | 8.87E−05 | 0.15 | KCNMA1 | 2.99 | 1.03E−02 | 0.27 | VPS11 | 2.08 | 1.05E−01 | 0.11 |
| SH3GL1 | 6.64 | 3.25E−10 | 0.4 | OSTC | 4.31 | 8.84E−05 | 0.13 | TRAPPC4 | 2.99 | 1.02E−02 | 0.19 | C11orf65 | 2.08 | 1.06E−01 | 0.09 |
| UGT2B7 | 6.64 | 3.28E−10 | 0.24 | GATA5 | 4.3 | 9.32E−05 | 3.1 | B3GALNT1 | 2.99 | 1.02E−02 | 0.18 | MOB4 | 2.08 | 1.06E−01 | 0.09 |
| PEX5L | 6.63 | 3.54E−10 | 2.32 | FLYWCH2 | 4.3 | 9.21E−05 | 0.95 | ATP11B | 2.99 | 1.04E−02 | 0.13 | LCLAT1 | 2.08 | 1.04E−01 | 0.07 |
| LAMTOR5 | 6.63 | 3.47E−10 | 0.84 | LRRC75B | 4.3 | 9.41E−05 | 0.69 | CAD | 2.99 | 1.02E−02 | 0.12 | CDK2 | 2.08 | 1.04E−01 | 0.07 |
| SGSM3 | 6.63 | 3.45E−10 | 0.55 | APOLD1 | 4.3 | 9.25E−05 | 0.62 | ZNF573 | 2.99 | 1.02E−02 | 0.07 | TRMT61A | 2.08 | 1.05E−01 | 0.07 |
| LMAN2 | 6.63 | 3.50E−10 | 0.55 | ABCA12 | 4.3 | 9.24E−05 | 0.55 | LMBR1L | 2.99 | 1.03E−02 | 0.05 | DNAH1 | 2.08 | 1.06E−01 | 0.03 |
| NRBP1 | 6.63 | 3.53E−10 | 0.4 | POLE4 | 4.3 | 9.10E−05 | 0.52 | MIR181A2HG | 2.99 | 1.02E−02 | 0.03 | STAP2 | 2.08 | 1.06E−01 | 0.01 |
| DSE | 6.62 | 3.68E−10 | 0.57 | ATRN | 4.3 | 9.24E−05 | 0.23 | DCTN5 | 2.99 | 1.02E−02 | 0.02 | ZW10 | 2.08 | 1.05E−01 | −0.01 |
| AHCYL1 | 6.62 | 3.69E−10 | 0.33 | NOD1 | 4.3 | 9.10E−05 | 0.19 | MTRR | 2.99 | 1.04E−02 | 0.02 | VAMP3 | 2.08 | 1.05E−01 | −0.05 |
| ECH1 | 6.61 | 3.91E−10 | 0.78 | PTPN13 | 4.3 | 9.36E−05 | 0.17 | ZNF720 | 2.99 | 1.03E−02 | 0.01 | SARIB | 2.08 | 1.06E−01 | −0.08 |
| COASY | 6.61 | 3.88E−10 | 0.48 | PREPL | 4.3 | 9.29E−05 | 0.1 | CARM1 | 2.99 | 1.04E−02 | 0 | ITGB3BP | 2.08 | 1.06E−01 | −0.1 |
| DHDDS | 6.61 | 3.94E−10 | 0.48 | ZDHHC5 | 4.3 | 9.41E−05 | 0.1 | LSS | 2.99 | 1.03E−02 | −0.02 | EBAG9 | 2.08 | 1.05E−01 | −0.11 |
| PSMB2 | 6.61 | 3.99E−10 | 0.47 | SMARCA1 | 4.3 | 9.31E−05 | 0.05 | GNAI2 | 2.99 | 1.04E−02 | −0.03 | BBIP1 | 2.08 | 1.05E−01 | −0.19 |
| DISC1 | 6.61 | 3.85E−10 | 0.39 | CNTNAP5 | 4.29 | 9.47E−05 | 0.92 | DNAI1 | 2.98 | 1.06E−02 | 1.31 | HSD11B1 | 2.07 | 1.09E−01 | 1.88 |
| TRPM4 | 6.61 | 3.91E−10 | 0.36 | GATC | 4.29 | 9.55E−05 | 0.83 | HAAO | 2.98 | 1.05E−02 | 0.92 | CIART | 2.07 | 1.09E−01 | 0.78 |
| DDX18 | 6.61 | 3.85E−10 | 0.35 | AC026202.3 | 4.29 | 9.48E−05 | 0.42 | ZNF134 | 2.98 | 1.05E−02 | 0.91 | TTC23L | 2.07 | 1.09E−01 | 0.71 |
| PLEKHB2 | 6.61 | 3.97E−10 | 0.32 | NARFL | 4.29 | 9.54E−05 | 0.31 | RGS19 | 2.98 | 1.05E−02 | 0.84 | TMEM147-AS1 | 2.07 | 1.09E−01 | 0.64 |
| MAGEF1 | 6.6 | 4.30E−10 | 1.29 | POLR3B | 4.29 | 9.55E−05 | 0.26 | PRPS1 | 2.98 | 1.07E−02 | 0.77 | SLC16A9 | 2.07 | 1.08E−01 | 0.4 |
| EDARADD | 6.6 | 4.24E−10 | 1.19 | CCDC186 | 4.29 | 9.46E−05 | 0.15 | DNAJC27-AS1 | 2.98 | 1.06E−02 | 0.74 | ADGRE3 | 2.07 | 1.07E−01 | 0.25 |
| CEP83 | 6.6 | 4.11E−10 | 0.39 | WIPI2 | 4.29 | 9.44E−05 | 0.14 | ATP12A | 2.98 | 1.07E−02 | 0.69 | TLR1 | 2.07 | 1.09E−01 | 0.24 |
| AKT2 | 6.6 | 4.32E−10 | 0.36 | WDR61 | 4.29 | 9.53E−05 | 0.14 | CNP | 2.98 | 1.06E−02 | 0.51 | GPKOW | 2.07 | 1.08E−01 | 0.19 |
| C7orf50 | 6.6 | 4.34E−10 | 0.34 | SUMO1 | 4.29 | 9.69E−05 | 0.1 | POLR3F | 2.98 | 1.05E−02 | 0.26 | SEC31A | 2.07 | 1.07E−01 | 0.08 |
| CCDC146 | 6.6 | 4.28E−10 | 0.33 | BPGM | 4.28 | 1.00E−04 | 1.09 | ZDHHC8 | 2.98 | 1.05E−02 | 0.24 | SCAPER | 2.07 | 1.08E−01 | 0.07 |
| IRF7 | 6.59 | 4.62E−10 | 0.78 | SGSM1 | 4.28 | 9.89E−05 | 0.78 | HINFP | 2.98 | 1.07E−02 | 0.2 | C9orf72 | 2.07 | 1.07E−01 | 0.03 |
| FRMD4A | 6.59 | 4.43E−10 | 0.43 | MYLK-AS1 | 4.28 | 1.01E−04 | 0.41 | PTAFR | 2.98 | 1.07E−02 | 0.18 | PAQR3 | 2.07 | 1.08E−01 | 0.01 |
| RPL7 | 6.59 | 4.44E−10 | 0.41 | RUNDC1 | 4.28 | 9.90E−05 | 0.33 | TWSG1 | 2.98 | 1.07E−02 | 0.14 | HSF4 | 2.07 | 1.08E−01 | −0.02 |
| ZSWIM4 | 6.59 | 4.47E−10 | 0.33 | SGTA | 4.28 | 9.85E−05 | 0.24 | DHCR24 | 2.98 | 1.06E−02 | 0.12 | ANXA5 | 2.07 | 1.09E−01 | −0.04 |
| IQCE | 6.59 | 4.47E−10 | 0.33 | C1orf52 | 4.28 | 1.03E−04 | 0.17 | SLC25A25 | 2.98 | 1.07E−02 | 0.1 | DPCD | 2.07 | 1.09E−01 | −0.07 |
| ITPR2 | 6.59 | 4.47E−10 | 0.3 | BTBD2 | 4.28 | 9.97E−05 | 0.16 | TTYH3 | 2.98 | 1.06E−02 | 0.08 | EMC1 | 2.07 | 1.07E−01 | −0.1 |
| NCKAP5L | 6.58 | 4.89E−10 | 0.92 | DOC2A | 4.27 | 1.07E−04 | 1.36 | LINC01515 | 2.98 | 1.05E−02 | 0.07 | CLK3 | 2.07 | 1.09E−01 | −0.13 |
| CFAP36 | 6.58 | 4.64E−10 | 0.39 | DCC | 4.27 | 1.04E−04 | 1.27 | HPCAL1 | 2.98 | 1.08E−02 | 0.07 | DGKK | 2.06 | 1.11E−01 | 1.44 |
| ASCC1 | 6.58 | 4.75E−10 | 0.3 | C2 | 4.27 | 1.03E−04 | 1.01 | FUT11 | 2.98 | 1.06E−02 | 0.04 | DAAM2 | 2.06 | 1.11E−01 | 1.27 |
| NDUFA2 | 6.57 | 5.18E−10 | 1.23 | SPESP1 | 4.27 | 1.04E−04 | 0.63 | ZNF136 | 2.98 | 1.06E−02 | −0.04 | KIAA1024 | 2.06 | 1.11E−01 | 1.22 |
| NENF | 6.56 | 5.33E−10 | 1.05 | DDX51 | 4.27 | 1.05E−04 | 0.58 | TSPEAR | 2.97 | 1.08E−02 | 1.89 | RAB24 | 2.06 | 1.09E−01 | 1.03 |
| MRPS7 | 6.56 | 5.34E−10 | 1.01 | COX5A | 4.27 | 1.05E−04 | 0.48 | GPC3 | 2.97 | 1.10E−02 | 0.93 | CXorf40B | 2.06 | 1.09E−01 | 0.94 |
| TYMP | 6.56 | 5.37E−10 | 0.48 | DNAJC8 | 4.27 | 1.03E−04 | 0.36 | HTRA2 | 2.97 | 1.08E−02 | 0.61 | NDUFAF3 | 2.06 | 1.11E−01 | 0.7 |
| FBRSL1 | 6.56 | 5.37E−10 | 0.38 | TOR1B | 4.27 | 1.07E−04 | 0.35 | ALG12 | 2.97 | 1.08E−02 | 0.56 | RBM43 | 2.06 | 1.11E−01 | 0.53 |
| KIAA0930 | 6.55 | 5.91E−10 | 0.65 | ZNF140 | 4.27 | 1.06E−04 | 0.3 | GPN1 | 2.97 | 1.09E−02 | 0.31 | ZNF646 | 2.06 | 1.10E−01 | 0.52 |
| LRRC59 | 6.55 | 5.99E−10 | 0.65 | EIF2AK4 | 4.27 | 1.04E−04 | 0.19 | MRFAPIL1 | 2.97 | 1.10E−02 | 0.24 | DOK6 | 2.06 | 1.10E−01 | 0.4 |
| SPDYE2 | 6.55 | 5.76E−10 | 0.54 | ACAP3 | 4.27 | 1.07E−04 | 0.17 | SGCE | 2.97 | 1.09E−02 | 0.17 | C11orf71 | 2.06 | 1.09E−01 | 0.37 |
| SPDYE3 | 6.55 | 5.95E−10 | 0.47 | RAB21 | 4.27 | 1.04E−04 | 0.14 | ASB1 | 2.97 | 1.09E−02 | 0.1 | ZSCAN9 | 2.06 | 1.10E−01 | 0.11 |
| PRKG1 | 6.55 | 5.68E−10 | 0.35 | SRSF3 | 4.27 | 1.06E−04 | 0.14 | TMEM44 | 2.97 | 1.09E−02 | 0.09 | ZNF316 | 2.06 | 1.09E−01 | 0.07 |
| GATAD2A | 6.55 | 5.95E−10 | 0.31 | GNB5 | 4.27 | 1.03E−04 | 0.13 | NEIL1 | 2.97 | 1.08E−02 | 0.05 | GSTCD | 2.06 | 1.11E−01 | 0 |
| C17orf99 | 6.54 | 6.40E−10 | 3.19 | WNT10A | 4.26 | 1.08E−04 | 1.13 | CDS2 | 2.97 | 1.09E−02 | 0.05 | LSM7 | 2.06 | 1.10E−01 | −0.01 |
| GADD45A | 6.54 | 6.02E−10 | 0.76 | MMP15 | 4.26 | 1.11E−04 | 0.72 | ZSWIM8 | 2.97 | 1.10E−02 | 0.02 | DDX11 | 2.06 | 1.11E−01 | −0.03 |
| SLC3A2 | 6.54 | 6.23E−10 | 0.42 | MRPL27 | 4.26 | 1.10E−04 | 0.43 | JAKMIP2 | 2.96 | 1.14E−02 | 1.27 | APTR | 2.06 | 1.09E−01 | −0.04 |
| DLG5 | 6.54 | 6.04E−10 | 0.33 | PPIL3 | 4.26 | 1.10E−04 | 0.36 | TMEM98 | 2.96 | 1.11E−02 | 0.54 | ALKBH3 | 2.06 | 1.10E−01 | −0.06 |
| ABCA13 | 6.53 | 6.65E−10 | 0.48 | CDC37 | 4.26 | 1.08E−04 | 0.14 | HMBS | 2.96 | 1.12E−02 | 0.51 | MBTPS1 | 2.06 | 1.11E−01 | −0.06 |
| RNPS1 | 6.53 | 6.74E−10 | 0.29 | ADCY5 | 4.25 | 1.13E−04 | 2.13 | PPT1 | 2.96 | 1.12E−02 | 0.38 | CC2D2A | 2.06 | 1.10E−01 | −0.08 |
| PGM2L1 | 6.53 | 6.49E−10 | 0.28 | LINC01268 | 4.25 | 1.15E−04 | 1.81 | BRD3 | 2.96 | 1.13E−02 | 0.1 | TWF1 | 2.06 | 1.09E−01 | −0.09 |
| KCNT2 | 6.52 | 6.85E−10 | 1.41 | TFPT | 4.25 | 1.14E−04 | 0.98 | TMEM51-AS1 | 2.96 | 1.13E−02 | 0.07 | CASP7 | 2.06 | 1.09E−01 | −0.1 |
| NT5E | 6.52 | 7.01E−10 | 1 | VPS25 | 4.25 | 1.16E−04 | 0.74 | UNKL | 2.96 | 1.13E−02 | 0.07 | HDAC2 | 2.06 | 1.09E−01 | −0.11 |
| BLVRA | 6.52 | 7.29E−10 | 0.84 | MTCL1 | 4.25 | 1.15E−04 | 0.25 | SEC23B | 2.96 | 1.13E−02 | 0.02 | AFAP1-AS1 | 2.06 | 1.10E−01 | −0.13 |
| TAOK3 | 6.52 | 7.20E−10 | 0.26 | ADH5 | 4.25 | 1.14E−04 | 0.22 | WFS1 | 2.96 | 1.11E−02 | −0.1 | AKAP8 | 2.06 | 1.10E−01 | −0.13 |
| SYVN1 | 6.51 | 7.61E−10 | 0.63 | TUBB6 | 4.25 | 1.13E−04 | 0.19 | SOX12 | 2.95 | 1.17E−02 | 1.12 | DNAJC24 | 2.06 | 1.11E−01 | −0.15 |
| COMT | 6.51 | 7.56E−10 | 0.38 | AGO2 | 4.25 | 1.14E−04 | 0.19 | SLC5A9 | 2.95 | 1.16E−02 | 0.98 | SLC16A12 | 2.05 | 1.11E−01 | 2.07 |
| VEZT | 6.51 | 7.38E−10 | 0.25 | TPP1 | 4.25 | 1.15E−04 | 0.19 | USH2A | 2.95 | 1.17E−02 | 0.92 | CA5A | 2.05 | 1.13E−01 | 1.76 |
| AK1 | 6.5 | 8.07E−10 | 1.82 | ZNF688 | 4.24 | 1.17E−04 | 1.25 | PSMB6 | 2.95 | 1.15E−02 | 0.81 | STAB2 | 2.05 | 1.13E−01 | 1.32 |
| SYT17 | 6.5 | 7.97E−10 | 0.32 | VWA1 | 4.24 | 1.18E−04 | 0.61 | FIBP | 2.95 | 1.15E−02 | 0.72 | DNAH10 | 2.05 | 1.13E−01 | 1.17 |
| NRXN1 | 6.49 | 8.48E−10 | 1.43 | PIN1 | 4.24 | 1.18E−04 | 0.48 | TNFRSF11B | 2.95 | 1.16E−02 | 0.52 | MEIS3 | 2.05 | 1.13E−01 | 0.73 |
| TIMM8B | 6.49 | 8.60E−10 | 1.34 | DUOX1 | 4.24 | 1.19E−04 | 0.2 | GPR137 | 2.95 | 1.14E−02 | 0.15 | PCIF1 | 2.05 | 1.13E−01 | 0.5 |
| ORAI2 | 6.49 | 8.48E−10 | 0.78 | TIMMDC1 | 4.24 | 1.19E−04 | 0.17 | KAT5 | 2.95 | 1.17E−02 | 0.15 | SLCO1B1 | 2.05 | 1.13E−01 | 0.35 |
| ERLEC1 | 6.49 | 8.72E−10 | 0.63 | CALU | 4.24 | 1.20E−04 | 0.14 | MIRLET7BHG | 2.95 | 1.14E−02 | 0.13 | PODXL2 | 2.05 | 1.12E−01 | 0.22 |
| ISG20 | 6.49 | 8.43E−10 | 0.54 | HS6ST3 | 4.23 | 1.21E−04 | 1.03 | SLC39A1 | 2.95 | 1.15E−02 | 0.13 | ANO1 | 2.05 | 1.12E−01 | 0.13 |
| CYB5R3 | 6.49 | 8.69E−10 | 0.4 | DEDD2 | 4.23 | 1.22E−04 | 0.49 | SMIM12 | 2.95 | 1.14E−02 | 0.12 | XYLT2 | 2.05 | 1.13E−01 | 0.09 |
| ECHDC2 | 6.49 | 8.32E−10 | 0.29 | SNRPE | 4.23 | 1.24E−04 | 0.41 | IRAK4 | 2.95 | 1.17E−02 | 0.08 | ANKS6 | 2.05 | 1.13E−01 | 0.03 |
| USF3 | 6.48 | 9.19E−10 | 0.44 | TBC1D10A | 4.23 | 1.23E−04 | 0.36 | CLTB | 2.95 | 1.16E−02 | 0.05 | EXT1 | 2.05 | 1.12E−01 | 0 |
| PPIL2 | 6.48 | 9.07E−10 | 0.29 | TSPAN17 | 4.23 | 1.22E−04 | 0.33 | CAMLG | 2.95 | 1.17E−02 | 0.01 | BRD9 | 2.05 | 1.12E−01 | −0.01 |
| NUP107 | 6.48 | 9.39E−10 | 0.29 | ZNF598 | 4.23 | 1.24E−04 | 0.33 | QPRT | 2.94 | 1.19E−02 | 2.24 | ZNF862 | 2.05 | 1.12E−01 | −0.03 |
| CTNNA2 | 6.47 | 9.81E−10 | 1.13 | NSMCE1 | 4.23 | 1.23E−04 | 0.31 | NRSN2 | 2.94 | 1.19E−02 | 1.6 | DECR2 | 2.05 | 1.13E−01 | −0.05 |
| PLPP3 | 6.46 | 1.06E−09 | 0.92 | JAG1 | 4.23 | 1.25E−04 | 0.29 | U91319.1 | 2.94 | 1.18E−02 | 1.1 | MUT | 2.05 | 1.12E−01 | −0.07 |
| DDAH2 | 6.46 | 1.02E−09 | 0.77 | DGKE | 4.23 | 1.22E−04 | 0.2 | ZNF275 | 2.94 | 1.18E−02 | 0.85 | STX10 | 2.05 | 1.13E−01 | −0.12 |
| GADD45B | 6.46 | 1.03E−09 | 0.72 | FNIP2 | 4.23 | 1.24E−04 | 0.17 | CCDC157 | 2.94 | 1.19E−02 | 0.83 | DRC7 | 2.04 | 1.14E−01 | 2.07 |
| SRD5A1 | 6.46 | 1.04E−09 | 0.45 | POC1B | 4.23 | 1.25E−04 | 0.15 | RBP1 | 2.94 | 1.20E−02 | 0.76 | IGF2BP1 | 2.04 | 1.14E−01 | 1.48 |
| ANAPC11 | 6.45 | 1.13E−09 | 1 | COL23A1 | 4.22 | 1.28E−04 | 1.55 | ZNF559.ZNF177 | 2.94 | 1.19E−02 | 0.62 | SCNN1G | 2.04 | 1.14E−01 | 1.29 |
| MRPL28 | 6.45 | 1.13E−09 | 0.9 | ADAMTS2 | 4.22 | 1.29E−04 | 0.62 | HPN | 2.94 | 1.18E−02 | 0.6 | ST6GALNAC4 | 2.04 | 1.16E−01 | 0.89 |
| COX6C | 6.45 | 1.11E−09 | 0.62 | COQ7 | 4.22 | 1.27E−04 | 0.49 | PATL2 | 2.94 | 1.19E−02 | 0.58 | SLC1A4 | 2.04 | 1.15E−01 | 0.77 |
| CYHR1 | 6.45 | 1.09E−09 | 0.41 | YIPF3 | 4.22 | 1.30E−04 | 0.44 | WDR62 | 2.94 | 1.19E−02 | 0.57 | ZNF668 | 2.04 | 1.15E−01 | 0.57 |
| DHX34 | 6.45 | 1.10E−09 | 0.39 | IMP4 | 4.22 | 1.28E−04 | 0.41 | ZNF737 | 2.94 | 1.20E−02 | 0.45 | WDR24 | 2.04 | 1.14E−01 | 0.5 |
| AARS | 6.45 | 1.08E−09 | 0.36 | SNX33 | 4.22 | 1.29E−04 | 0.4 | VTI1B | 2.94 | 1.19E−02 | 0.38 | PIP5KL1 | 2.04 | 1.16E−01 | 0.49 |
| TP53I11 | 6.44 | 1.21E−09 | 1.11 | TMUB1 | 4.22 | 1.27E−04 | 0.35 | PLBD1-AS1 | 2.94 | 1.18E−02 | 0.28 | TFAP4 | 2.04 | 1.16E−01 | 0.49 |
| SIDT2 | 6.44 | 1.19E−09 | 0.59 | ME1 | 4.22 | 1.29E−04 | 0.35 | RNFT1 | 2.94 | 1.18E−02 | 0.17 | ZNF446 | 2.04 | 1.14E−01 | 0.48 |
| PRICKLE1 | 6.44 | 1.16E−09 | 0.4 | APH1B | 4.22 | 1.30E−04 | 0.35 | PMVK | 2.94 | 1.20E−02 | 0.14 | LYSMD2 | 2.04 | 1.14E−01 | 0.46 |
| GLIS2 | 6.43 | 1.26E−09 | 1.02 | GMPPA | 4.22 | 1.31E−04 | 0.32 | HMGN2 | 2.94 | 1.20E−02 | 0.12 | CENPN | 2.04 | 1.14E−01 | 0.43 |
| PHLDA3 | 6.43 | 1.27E−09 | 0.71 | TRMT1 | 4.22 | 1.30E−04 | 0.21 | CRELD1 | 2.94 | 1.21E−02 | 0.08 | CDKN2AIPNL | 2.04 | 1.14E−01 | 0.37 |
| LMO4 | 6.43 | 1.23E−09 | 0.41 | STK17B | 4.22 | 1.30E−04 | 0.16 | PPID | 2.94 | 1.18E−02 | 0.04 | ALS2CR12 | 2.04 | 1.15E−01 | 0.36 |
| CD40 | 6.42 | 1.36E−09 | 1.27 | GNL1 | 4.22 | 1.27E−04 | 0.12 | NRG4 | 2.94 | 1.20E−02 | 0.02 | MED8 | 2.04 | 1.15E−01 | 0.25 |
| B9D1 | 6.42 | 1.37E−09 | 1.12 | OPTN | 4.22 | 1.29E−04 | 0.12 | ARNTL | 2.94 | 1.20E−02 | 0.01 | NBPF3 | 2.04 | 1.15E−01 | 0.23 |
| PCAT7 | 6.42 | 1.33E−09 | 0.94 | C12orf75 | 4.22 | 1.27E−04 | 0.09 | CTR9 | 2.94 | 1.20E−02 | 0 | SPATA2L | 2.04 | 1.14E−01 | 0.15 |
| PRELID1 | 6.42 | 1.30E−09 | 0.71 | SMPD1 | 4.21 | 1.36E−04 | 1.36 | SERINC1 | 2.94 | 1.18E−02 | −0.05 | CEP85 | 2.04 | 1.14E−01 | 0.07 |
| PRKAB1 | 6.42 | 1.34E−09 | 0.47 | AC007099.1 | 4.21 | 1.33E−04 | 1 | RASSF9 | 2.93 | 1.21E−02 | 0.67 | PJA2 | 2.04 | 1.15E−01 | −0.01 |
| IRAK3 | 6.42 | 1.34E−09 | 0.32 | STK31 | 4.21 | 1.33E−04 | 0.72 | TM2D2 | 2.93 | 1.23E−02 | 0.47 | BOD1 | 2.04 | 1.14E−01 | −0.02 |
| SEC14L1 | 6.42 | 1.32E−09 | 0.3 | SF3B5 | 4.21 | 1.32E−04 | 0.66 | TMEM106C | 2.93 | 1.24E−02 | 0.27 | CEP68 | 2.04 | 1.14E−01 | −0.03 |
| CATSPERG | 6.41 | 1.41E−09 | 0.69 | FTSJ1 | 4.21 | 1.34E−04 | 0.48 | SLC40A1 | 2.93 | 1.21E−02 | 0.21 | PIGK | 2.04 | 1.14E−01 | −0.05 |
| HMOX2 | 6.41 | 1.44E−09 | 0.43 | WDR72 | 4.21 | 1.36E−04 | 0.42 | TRMT2A | 2.93 | 1.24E−02 | 0.19 | ZCCHC10 | 2.04 | 1.14E−01 | −0.07 |
| GNG5 | 6.41 | 1.41E−09 | 0.42 | E4F1 | 4.21 | 1.33E−04 | 0.26 | FAM129B | 2.93 | 1.23E−02 | 0.12 | KHSRP | 2.04 | 1.14E−01 | −0.13 |
| SART3 | 6.41 | 1.46E−09 | 0.38 | ABCF1 | 4.21 | 1.37E−04 | 0.19 | U2AF2 | 2.93 | 1.24E−02 | 0.12 | ATAT1 | 2.04 | 1.15E−01 | −0.15 |
| MRPS12 | 6.4 | 1.53E−09 | 1.51 | SMG6 | 4.21 | 1.32E−04 | 0.18 | ZNF19 | 2.93 | 1.23E−02 | 0.1 | KCNB1 | 2.03 | 1.18E−01 | 1.32 |
| MAF1 | 6.4 | 1.53E−09 | 0.91 | PSMG2 | 4.21 | 1.37E−04 | 0.16 | ATPAF1 | 2.93 | 1.21E−02 | 0.08 | MAST1 | 2.03 | 1.17E−01 | 0.67 |
| CUTA | 6.4 | 1.50E−09 | 0.82 | NUP93 | 4.21 | 1.33E−04 | 0.15 | ZNF846 | 2.93 | 1.24E−02 | 0.06 | RELL2 | 2.03 | 1.17E−01 | 0.65 |
| RETSAT | 6.4 | 1.49E−09 | 0.49 | TTC1 | 4.21 | 1.35E−04 | 0.13 | DNAJC2 | 2.93 | 1.24E−02 | 0.04 | CNBD2 | 2.03 | 1.18E−01 | 0.65 |
| PSMC2 | 6.4 | 1.53E−09 | 0.48 | CEP70 | 4.21 | 1.32E−04 | 0.1 | C3orf33 | 2.92 | 1.26E−02 | 2.25 | ADPRHL1 | 2.03 | 1.17E−01 | 0.57 |
| POLR1A | 6.4 | 1.47E−09 | 0.45 | B3GALT5-AS1 | 4.2 | 1.39E−04 | 1.38 | RNF26 | 2.92 | 1.26E−02 | 1.18 | MINCR | 2.03 | 1.17E−01 | 0.39 |
| RNH1 | 6.4 | 1.55E−09 | 0.35 | CPS1 | 4.2 | 1.38E−04 | 1 | GSTT2B | 2.92 | 1.26E−02 | 1.01 | DNAJC6 | 2.03 | 1.17E−01 | 0.36 |
| PLAG1 | 6.4 | 1.54E−09 | 0.34 | LINC00882 | 4.2 | 1.42E−04 | 0.69 | MAPK7 | 2.92 | 1.27E−02 | 0.8 | ZNF200 | 2.03 | 1.18E−01 | 0.31 |
| NIPA2 | 6.4 | 1.53E−09 | 0.32 | HOXA-AS2 | 4.2 | 1.41E−04 | 0.58 | ROR1-AS1 | 2.92 | 1.27E−02 | 0.6 | GBP4 | 2.03 | 1.16E−01 | 0.27 |
| CX3CL1 | 6.39 | 1.63E−09 | 1.57 | LRRC4C | 4.2 | 1.38E−04 | 0.49 | FAM207A | 2.92 | 1.27E−02 | 0.49 | ISM1 | 2.03 | 1.18E−01 | 0.26 |
| CNDP2 | 6.39 | 1.62E−09 | 0.43 | C1orf54 | 4.2 | 1.43E−04 | 0.3 | HNRNPH2 | 2.92 | 1.28E−02 | 0.44 | SYDE2 | 2.03 | 1.17E−01 | 0.09 |
| ERGIC1 | 6.39 | 1.63E−09 | 0.31 | ACACB | 4.2 | 1.39E−04 | 0.17 | CDKN1C | 2.92 | 1.28E−02 | 0.37 | ABHD8 | 2.03 | 1.17E−01 | 0.08 |
| ANAPC5 | 6.39 | 1.65E−09 | 0.28 | PER2 | 4.2 | 1.40E−04 | 0.16 | MPLKIP | 2.92 | 1.25E−02 | 0.2 | ALDH9A1 | 2.03 | 1.17E−01 | 0.07 |
| AC008074.3 | 6.38 | 1.66E−09 | 3.89 | SIVA1 | 4.19 | 1.48E−04 | 0.64 | PLCB3 | 2.92 | 1.27E−02 | 0.18 | NTN1 | 2.03 | 1.18E−01 | 0.06 |
| YKT6 | 6.38 | 1.67E−09 | 0.77 | LRGUK | 4.19 | 1.49E−04 | 0.52 | HEXIM1 | 2.92 | 1.27E−02 | 0.15 | MAGOHB | 2.03 | 1.18E−01 | 0 |
| MEM01 | 6.38 | 1.74E−09 | 0.65 | ERO1B | 4.19 | 1.49E−04 | 0.5 | NRG3 | 2.92 | 1.26E−02 | 0.09 | HINT3 | 2.03 | 1.18E−01 | 0 |
| UBTD1 | 6.38 | 1.74E−09 | 0.59 | PA2G4 | 4.19 | 1.45E−04 | 0.35 | ZNF480 | 2.92 | 1.26E−02 | 0.09 | LRRC37B | 2.03 | 1.17E−01 | −0.03 |
| CCDC65 | 6.38 | 1.69E−09 | 0.52 | RASSF4 | 4.19 | 1.47E−04 | 0.35 | AMPD2 | 2.92 | 1.26E−02 | 0.09 | BANP | 2.03 | 1.16E−01 | −0.04 |
| PTPRH | 6.38 | 1.66E−09 | 0.47 | PDE4A | 4.19 | 1.45E−04 | 0.34 | RHOB | 2.92 | 1.28E−02 | 0.09 | MSH2 | 2.03 | 1.17E−01 | −0.04 |
| ATP6V1E1 | 6.38 | 1.75E−09 | 0.47 | RECQL | 4.19 | 1.47E−04 | 0.19 | ZNF490 | 2.92 | 1.27E−02 | 0.06 | DHX8 | 2.03 | 1.16E−01 | −0.06 |
| TMEM104 | 6.37 | 1.84E−09 | 0.99 | CCDC82 | 4.19 | 1.47E−04 | 0.13 | SMS | 2.92 | 1.27E−02 | 0.05 | PIGF | 2.03 | 1.17E−01 | −0.06 |
| SPATA20 | 6.37 | 1.84E−09 | 0.48 | ASIP | 4.18 | 1.51E−04 | 1.23 | ZNF765 | 2.92 | 1.28E−02 | 0.04 | DIS3 | 2.03 | 1.18E−01 | −0.07 |
| LIPE-AS1 | 6.37 | 1.78E−09 | 0.37 | PGRMC1 | 4.18 | 1.50E−04 | 0.51 | FAM84B | 2.92 | 1.27E−02 | 0.02 | DNAH11 | 2.03 | 1.16E−01 | −0.13 |
| HSP90AB1 | 6.37 | 1.83E−09 | 0.35 | E2F4 | 4.18 | 1.54E−04 | 0.21 | AC009313.1 | 2.91 | 1.32E−02 | 1.46 | TXNL4A | 2.03 | 1.18E−01 | −0.13 |
| SAMD3 | 6.36 | 1.97E−09 | 2.14 | ESR2 | 4.18 | 1.52E−04 | 0.17 | TCEAL1 | 2.91 | 1.30E−02 | 1.3 | YPEL3 | 2.03 | 1.16E−01 | −0.17 |
| DKK3 | 6.36 | 1.93E−09 | 1.87 | AKAP17A | 4.18 | 1.50E−04 | 0.16 | PLXNA4 | 2.91 | 1.30E−02 | 1.22 | ZG16B | 2.03 | 1.17E−01 | −0.2 |
| C12orf57 | 6.36 | 1.89E−09 | 1.37 | MUM1 | 4.18 | 1.54E−04 | 0.16 | LAT2 | 2.91 | 1.28E−02 | 0.74 | MIR7-3HG | 2.02 | 1.19E−01 | 2.95 |
| HSCB | 6.36 | 1.91E−09 | 0.62 | PHKG1 | 4.17 | 1.57E−04 | 0.6 | SNAP25-AS1 | 2.91 | 1.29E−02 | 0.72 | ECHDC3 | 2.02 | 1.19E−01 | 2.51 |
| CACNA1C | 6.36 | 1.89E−09 | 0.3 | CRELD2 | 4.17 | 1.57E−04 | 0.52 | CERKL | 2.91 | 1.30E−02 | 0.71 | ZMAT5 | 2.02 | 1.20E−01 | 0.58 |
| KIAA1671 | 6.36 | 1.91E−09 | 0.29 | SHKBP1 | 4.17 | 1.58E−04 | 0.43 | ZSCAN31 | 2.91 | 1.32E−02 | 0.51 | GRM7 | 2.02 | 1.19E−01 | 0.44 |
| MVP | 6.36 | 1.90E−09 | 0.28 | ANK2 | 4.17 | 1.58E−04 | 0.38 | PTCHD4 | 2.91 | 1.31E−02 | 0.41 | AXDND1 | 2.02 | 1.21E−01 | 0.41 |
| SLC30A8 | 6.35 | 2.04E−09 | 2.12 | SGK1 | 4.17 | 1.57E−04 | 0.31 | GCNT3 | 2.91 | 1.32E−02 | 0.17 | COA6 | 2.02 | 1.21E−01 | 0.33 |
| EMC8 | 6.35 | 2.08E−09 | 0.53 | CASP6 | 4.17 | 1.56E−04 | 0.23 | MGME1 | 2.91 | 1.30E−02 | 0.14 | TRMT10A | 2.02 | 1.19E−01 | 0.31 |
| CCDC24 | 6.35 | 2.12E−09 | 0.53 | SNHG12 | 4.17 | 1.57E−04 | 0.19 | NUDT19 | 2.91 | 1.29E−02 | 0.13 | ZNF273 | 2.02 | 1.18E−01 | 0.17 |
| MRPL20 | 6.35 | 2.12E−09 | 0.49 | SHISA5 | 4.17 | 1.60E−04 | 0.19 | SLIRP | 2.91 | 1.29E−02 | 0.11 | ENC1 | 2.02 | 1.21E−01 | 0.04 |
| AKR1A1 | 6.35 | 2.03E−09 | 0.39 | SLC25A29 | 4.17 | 1.57E−04 | 0.16 | CCDC149 | 2.91 | 1.30E−02 | 0.1 | VEGFB | 2.02 | 1.20E−01 | 0.01 |
| ANKS1B | 6.34 | 2.14E−09 | 1.14 | FAM214B | 4.17 | 1.58E−04 | 0.12 | MORF4L2 | 2.91 | 1.31E−02 | 0.1 | LINC01135 | 2.02 | 1.19E−01 | 0 |
| CSK | 6.34 | 2.18E−09 | 0.77 | KLC4 | 4.17 | 1.58E−04 | 0.11 | METTL22 | 2.91 | 1.30E−02 | 0.07 | FOXJ2 | 2.02 | 1.20E−01 | 0 |
| CFTR | 6.34 | 2.17E−09 | 0.65 | ANXA2 | 4.17 | 1.60E−04 | 0.06 | TAF12 | 2.91 | 1.31E−02 | 0.06 | FBXO33 | 2.02 | 1.19E−01 | −0.03 |
| HAT1 | 6.34 | 2.23E−09 | 0.4 | RPF2 | 4.16 | 1.68E−04 | 0.32 | TFCP2L1 | 2.91 | 1.31E−02 | 0.06 | UTP18 | 2.02 | 1.19E−01 | −0.04 |
| MT-CYB | 6.34 | 2.14E−09 | 0.39 | UROS | 4.16 | 1.66E−04 | 0.29 | SERAC1 | 2.91 | 1.30E−02 | 0.03 | PANX2 | 2.01 | 1.21E−01 | 1.58 |
| RAB7A | 6.34 | 2.24E−09 | 0.25 | AP1M2 | 4.16 | 1.67E−04 | 0.19 | LRRC42 | 2.91 | 1.29E−02 | −0.01 | ZNF599 | 2.01 | 1.21E−01 | 1.16 |
| ODF3B | 6.33 | 2.35E−09 | 0.88 | GNA12 | 4.16 | 1.67E−04 | 0.19 | TTL | 2.91 | 1.31E−02 | −0.01 | PAMI6 | 2.01 | 1.23E−01 | 1.08 |
| ASPSCR1 | 6.33 | 2.35E−09 | 0.45 | GRB2 | 4.16 | 1.64E−04 | 0.18 | LMLN | 2.91 | 1.30E−02 | −0.02 | CENPW | 2.01 | 1.22E−01 | 1.03 |
| RNF40 | 6.33 | 2.36E−09 | 0.45 | MPPED2 | 4.15 | 1.72E−04 | 2.06 | IL2RG | 2.9 | 1.35E−02 | 3.76 | DACH2 | 2.01 | 1.21E−01 | 0.8 |
| TNPO2 | 6.33 | 2.29E−09 | 0.37 | LINC01609 | 4.15 | 1.72E−04 | 1.99 | RPH3A | 2.9 | 1.35E−02 | 1.57 | C19orf54 | 2.01 | 1.23E−01 | 0.38 |
| FBLIM1 | 6.33 | 2.35E−09 | 0.28 | IFITM10 | 4.15 | 1.74E−04 | 1.69 | EFR3B | 2.9 | 1.35E−02 | 1.28 | GDF11 | 2.01 | 1.23E−01 | 0.31 |
| HERC3 | 6.32 | 2.56E−09 | 0.34 | MLST8 | 4.15 | 1.74E−04 | 0.65 | CECR2 | 2.9 | 1.34E−02 | 0.92 | DRC3 | 2.01 | 1.21E−01 | 0.27 |
| TRIM54 | 6.31 | 2.66E−09 | 1.71 | KEAP1 | 4.15 | 1.73E−04 | 0.38 | MSRB1 | 2.9 | 1.33E−02 | 0.81 | PLEKHG5 | 2.01 | 1.21E−01 | 0.11 |
| PSMD3 | 6.31 | 2.61E−09 | 0.5 | FIS1 | 4.15 | 1.73E−04 | 0.28 | OSBPL10-AS1 | 2.9 | 1.34E−02 | 0.44 | MYRF | 2.01 | 1.22E−01 | 0.08 |
| LINC00662 | 6.31 | 2.60E−09 | 0.49 | ARRDC2 | 4.15 | 1.72E−04 | 0.26 | PDGFD | 2.9 | 1.32E−02 | 0.25 | ATP5S | 2.01 | 1.22E−01 | −0.01 |
| BAG6 | 6.31 | 2.72E−09 | 0.46 | NOL11 | 4.15 | 1.71E−04 | 0.22 | TXNL1 | 2.9 | 1.33E−02 | 0.05 | IMMP1L | 2.01 | 1.22E−01 | −0.04 |
| CRY2 | 6.31 | 2.70E−09 | 0.43 | POFUT2 | 4.15 | 1.70E−04 | 0.2 | RNPEP | 2.9 | 1.34E−02 | 0.01 | PAIP2 | 2.01 | 1.21E−01 | −0.06 |
| PSMD11 | 6.31 | 2.72E−09 | 0.34 | PPIP5K1 | 4.15 | 1.74E−04 | 0.19 | RNF212B | 2.89 | 1.38E−02 | 1.27 | GTF2H3 | 2.01 | 1.21E−01 | −0.08 |
| TSPAN4 | 6.3 | 2.75E−09 | 0.51 | CFAP69 | 4.15 | 1.70E−04 | 0.17 | CDIP1 | 2.89 | 1.37E−02 | 1.09 | ALAD | 2.01 | 1.23E−01 | −0.15 |
| LAMA2 | 6.3 | 2.77E−09 | 0.5 | DTNB | 4.15 | 1.75E−04 | 0.15 | TMEM136 | 2.89 | 1.36E−02 | 0.79 | POGK | 2.01 | 1.22E−01 | −0.2 |
| XPNPEP3 | 6.3 | 2.88E−09 | 0.36 | MYBPC1 | 4.14 | 1.82E−04 | 2.32 | RMDN2-AS1 | 2.89 | 1.36E−02 | 0.78 | LINC01591 | 2 | 1.26E−01 | 1.96 |
| NCEH1 | 6.3 | 2.83E−09 | 0.33 | ALK | 4.14 | 1.80E−04 | 1.1 | FSTL5 | 2.89 | 1.36E−02 | 0.71 | ALDH1B1 | 2 | 1.24E−01 | 1.58 |
| MGAM2 | 6.29 | 2.99E−09 | 0.75 | RHOG | 4.14 | 1.78E−04 | 0.82 | TYRO3 | 2.89 | 1.38E−02 | 0.66 | SNRPN | 2 | 1.25E−01 | 1.47 |
| FBXL18 | 6.29 | 2.97E−09 | 0.37 | TRMT10C | 4.14 | 1.80E−04 | 0.62 | PDE3A | 2.89 | 1.38E−02 | 0.37 | KCNN1 | 2 | 1.24E−01 | 1.15 |
| SLC15A2 | 6.28 | 3.25E−09 | 1.21 | PTGS2 | 4.14 | 1.78E−04 | 0.59 | MXRA7 | 2.89 | 1.39E−02 | 0.36 | SLA | 2 | 1.26E−01 | 0.74 |
| CD163L1 | 6.28 | 3.21E−09 | 0.82 | COQ5 | 4.14 | 1.79E−04 | 0.49 | FAM217B | 2.89 | 1.36E−02 | 0.21 | MRPL40 | 2 | 1.26E−01 | 0.71 |
| ACAT1 | 6.28 | 3.23E−09 | 0.77 | ERCC4 | 4.14 | 1.80E−04 | 0.37 | PDE1A | 2.89 | 1.36E−02 | 0.15 | ARMC5 | 2 | 1.26E−01 | 0.29 |
| DYNC1I1 | 6.28 | 3.10E−09 | 0.74 | CCHCR1 | 4.14 | 1.77E−04 | 0.33 | MRPL52 | 2.89 | 1.38E−02 | 0.1 | GLRX5 | 2 | 1.24E−01 | 0.19 |
| NDUFB4 | 6.28 | 3.24E−09 | 0.7 | HMCES | 4.14 | 1.77E−04 | 0.32 | CCM2 | 2.89 | 1.37E−02 | 0.09 | ASNS | 2 | 1.24E−01 | 0.08 |
| IRF2BP2 | 6.28 | 3.21E−09 | 0.33 | CDK16 | 4.14 | 1.80E−04 | 0.3 | MFAP3 | 2.89 | 1.36E−02 | 0.06 | UPP1 | 2 | 1.24E−01 | 0.07 |
| ANKRD36B | 6.28 | 3.17E−09 | 0.28 | ERAP2 | 4.14 | 1.80E−04 | 0.27 | AP3D1 | 2.89 | 1.38E−02 | 0.04 | MIDN | 2 | 1.24E−01 | 0.02 |
| TBC1D20 | 6.27 | 3.29E−09 | 0.69 | NLGN1 | 4.14 | 1.82E−04 | 0.27 | LSG1 | 2.89 | 1.40E−02 | 0.02 | GCKR | 2 | 1.26E−01 | 0.01 |
| DNAH3 | 6.27 | 3.30E−09 | 0.61 | PLCL2 | 4.14 | 1.77E−04 | 0.23 | PRC1 | 2.88 | 1.42E−02 | 1.47 | LUC7L | 2 | 1.24E−01 | 0 |
| LRRC23 | 6.27 | 3.42E−09 | 0.41 | MED27 | 4.14 | 1.79E−04 | 0.15 | STX16-NPEPL1 | 2.88 | 1.42E−02 | 0.69 | RPA2 | 2 | 1.26E−01 | −0.02 |
| TMEM123 | 6.27 | 3.36E−09 | 0.33 | NIPSNAP1 | 4.13 | 1.87E−04 | 0.52 | VSTM5 | 2.88 | 1.40E−02 | 0.66 | TNKS1BP1 | 2 | 1.24E−01 | −0.03 |
| PGM1 | 6.27 | 3.44E−09 | 0.28 | NR1H2 | 4.13 | 1.85E−04 | 0.32 | ASNA1 | 2.88 | 1.41E−02 | 0.36 | CDH3 | 2 | 1.26E−01 | −0.03 |
| LY6K | 6.26 | 3.58E−09 | 2.96 | PWWP2B | 4.13 | 1.83E−04 | 0.27 | AEN | 2.88 | 1.43E−02 | 0.36 | DNAJC16 | 2 | 1.25E−01 | −0.05 |
| PSMC3 | 6.26 | 3.56E−09 | 1.14 | POFUT1 | 4.13 | 1.87E−04 | 0.24 | ARL14 | 2.88 | 1.43E−02 | 0.19 | POLL | 2 | 1.24E−01 | −0.06 |
| SLC26A2 | 6.26 | 3.52E−09 | 1.03 | ECI1 | 4.13 | 1.90E−04 | 0.15 | TAB1 | 2.88 | 1.42E−02 | 0.15 | PLEKHA8 | 2 | 1.24E−01 | −0.11 |
| CBWD3 | 6.26 | 3.56E−09 | 0.64 | PTCHD1 | 4.12 | 1.96E−04 | 1.77 | SUMO2 | 2.88 | 1.43E−02 | 0.09 | INIP | 2 | 1.25E−01 | −0.11 |
| UHRF1BP1L | 6.26 | 3.60E−09 | 0.38 | SETD1A | 4.12 | 1.92E−04 | 0.48 | GRHL1 | 2.88 | 1.41E−02 | 0.05 | WDR19 | 2 | 1.25E−01 | −0.13 |
| P′CMT1 | 6.26 | 3.70E−09 | 0.3 | THRA | 4.12 | 1.94E−04 | 0.35 | SUDS3 | 2.88 | 1.41E−02 | 0.03 | HIF1AN | 2 | 1.25E−01 | −0.16 |
| LAMC1 | 6.26 | 3.66E−09 | 0.28 | C12orf49 | 4.12 | 1.98E−04 | 0.25 | BBS4 | 2.88 | 1.41E−02 | 0.02 | NUDT9 | 2 | 1.25E−01 | −0.17 |
| RHBDD3 | 6.25 | 3.75E−09 | 0.99 | SOGA1 | 4.12 | 1.95E−04 | 0.21 | DCLRE1C | 2.88 | 1.42E−02 | 0.02 | CEP104 | 2 | 1.25E−01 | −0.17 |
| HERPUD1 | 6.25 | 3.80E−09 | 0.38 | NEK1 | 4.12 | 1.97E−04 | 0.17 | DCTPP1 | 2.87 | 1.46E−02 | 0.66 | PIGC | 2 | 1.25E−01 | −0.22 |
| CEP164 | 6.25 | 3.90E−09 | 0.36 | PGRMC2 | 4.12 | 1.93E−04 | 0.16 | PDLIM3 | 2.87 | 1.47E−02 | 0.5 | | | | |
| S100PBP | 6.25 | 3.75E−09 | 0.33 | OSBPL2 | 4.12 | 1.98E−04 | 0.15 | POLN | 2.87 | 1.46E−02 | 0.47 | | | | |
| PRKD2 | 6.25 | 3.75E−09 | 0.31 | SLC44A4 | 4.12 | 1.97E−04 | 0.14 | TUBA1A | 2.87 | 1.45E−02 | 0.42 | | | | |
| MRPL51 | 6.24 | 4.11E−09 | 1.1 | SBNO1 | 4.12 | 1.98E−04 | 0.14 | HIST1H2BD | 2.87 | 1.45E−02 | 0.17 | | | | |
| RAB35 | 6.24 | 3.97E−09 | 0.69 | TMEM99 | 4.11 | 1.99E−04 | 0.8 | ZNF821 | 2.87 | 1.48E−02 | 0.13 | | | | |
| GRAMD4 | 6.24 | 4.10E−09 | 0.45 | CRAT | 4.11 | 2.02E−04 | 0.64 | ARID5B | 2.87 | 1.44E−02 | 0.08 | | | | |
| CIB1 | 6.24 | 4.16E−09 | 0.27 | CLMP | 4.11 | 2.04E−04 | 0.51 | C9orf41-AS1 | 2.86 | 1.50E−02 | 0.45 | | | | |
| BPTF | 6.24 | 3.98E−09 | 0.23 | DUS2 | 4.11 | 2.02E−04 | 0.22 | DNAH17 | 2.86 | 1.50E−02 | 0.41 | | | | |

Interferon Signaling and Immune-Promoting Responses in Malignant Cells after Treatment

Analysis of genes differentially expressed by malignant cells in untreated vs. treated tumors (FIG. 2C) revealed elevated expression of type I and II interferon (IFN) response genes following neoadjuvant CRT. This was detected even when cells from the two patients with germline BRCA2 mutations were removed from the treated group (PDAC_T_1, PDAC_T_2; Table 1; FIG. 9C). This phenomenon could be due to a release of damage associated molecular patterns (DAMPs) and pattern recognition receptors (PRRs) by CRT that converge on interferon signaling⁷¹⁻⁷³.

Notably, the IFN-γ pathway genes that are elevated in the malignant cells post treatment (FIG. 2C) appear to be associated with immune-promoting rather than immune suppressive programs. None of the nonclassical MHC class I (MHC-I) genes that are thought to be immunosuppressive were differentially overexpressed post-treatment; the classical HLA-B was the only MHC-I molecule that was higher in the post-treatment context (FIG. 2C)^{23,74}. Furthermore, inferred interactions between HLA-A, -B, or -C on malignant cells and CD3G on CD8⁺ T-cells were higher post-treatment (FIG. 2B). These data suggest that the addition of type I interferons may improve PDAC outcomes, although adjuvant CRT plus interferon alpha 2b did not improve survival compared to chemotherapy^{75,76}. Overall, these results are consistent with reports that CRT may enhance MHC-I expression for recognition by cytotoxic immune cells⁷⁷.

Subtype-Specific Treatment Response in Malignant Cells Suggests that Basal-Like Malignant Cells are More Resistant to CRT than Classical-Like Cells

The differentially expressed genes in malignant cells suggest a relative increase in basal-like cells and a decrease in classical-like cells in treated vs. untreated tumors. Genes differentially overexpressed in malignant cells from post-treatment tumors were enriched in the basal-like signature^{9,78}(p=2.05×10⁻⁶, hypergeometric test), and included TP63, a master transcription factor for the PDAC squamous subtype⁷⁹ (FIG. 2C). Conversely, differentially underexpressed genes were enriched for the classical-like signature (p=4.46×10⁻²⁴), including the hallmark transcription factor GATA6^{7,9,78,80} (FIG. 2C). Consistently, human PDAC cell lines become enriched for the basal-like subtype after FOLFIRINOX^{14,81}. These observations could reflect either cell state changes following CRT, differential sensitivity to CRT, or both. Notably, the response to first-line combination chemotherapy for advanced PDAC is significantly better in patients with GATA6-expressing classical subtype tumors, supporting a model of increased sensitivity of classical-like cells to CRT.

Furthermore, genes differentially expressed within the treated cohort between malignant cells from patients with high (≥10%) vs. low residual neoplastic content (FIG. 9D; Tables 1,2) were also associated with the basal-like vs. classical-like distinction. Specifically, genes elevated in the high residual group were enriched for the basal-like signature (p=8.34×10⁻⁸, hypergeometric test), and those underexpressed were enriched for the classical-like subtype (p=1.65×10⁻⁵¹) (FIG. 9D). Interestingly, MUC16, a source of neoantigens when mutated, and a potential target for chimeric antigen receptor (CAR) T cell therapy⁸², was elevated in high-residual tumors. This may provide rationale for testing immunomodulatory neoadjuvant strategies to complement CRT. Overall, this analysis supports a model of a subtype-dependent response to CRT, such that basal-like neoplastic cells are more resistant to cytotoxic therapy than classical-like neoplastic cells. Future studies of matched pre- and post-treatment tumors will help distinguish direct selection on existing cell states from treatment-induced state changes.

In malignant cells, treatment was also correlated with distinct expression of genes needed to maintain the Wnt/β-catenin niche, which is crucial to treatment resistance, and can be mediated by either paracrine interactions with CAFs or autocrine signaling by malignant cells.⁸³ The expression of the Wnt family receptor LRP5 was sustained in surviving malignant cells post-treatment, but there appeared to be shifts in the source of Wnt signaling. CRT was associated with a concomitant differential underexpression in WNT5A by CAFs and overexpression in autocrine WNT7B signaling by malignant cells (FIGS. 2B-2C), which has previously been shown to drive anchorage-independent growth and worse disease-specific survival in PDAC.⁸⁴

### Novel Malignant Cell Programs Reveal a Refined Molecular Classification

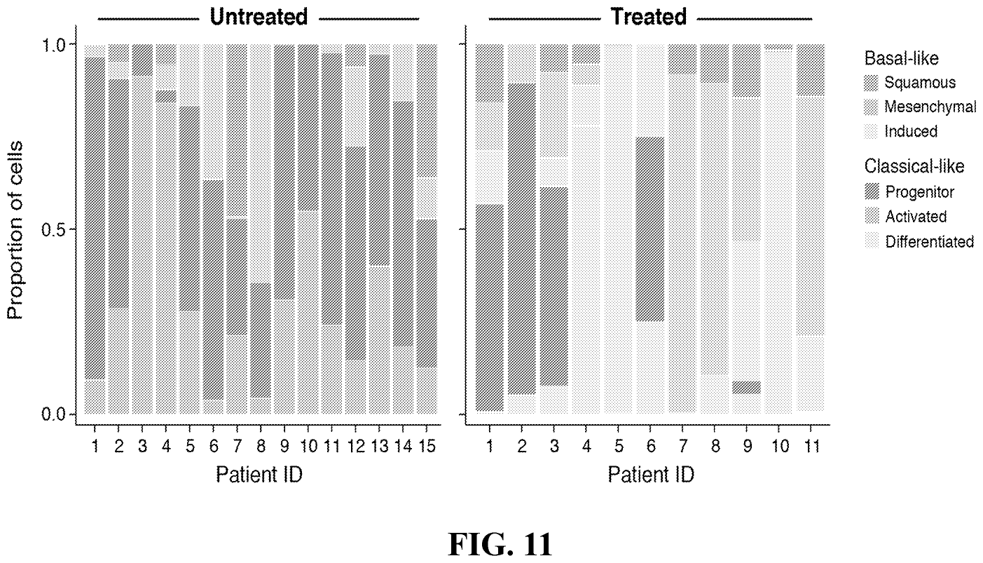

Next, it was sought to better characterize expression states within the malignant cells across patients. Consistent with recent reports of intra-tumoral subtype heterogeneity, nearly every untreated tumor contained both basal-like and classical-like cells (FIG. 11; Methods)^{78,85}, with the two states being largely mutually exclusive. In the treated cohort, bulk signatures overlapped in the same nucleus, suggesting that the basal-like and classical-like signatures derived in the treatment-naive setting may be less relevant in the neoadjuvant treatment context. This further supports the possibility of state changes and highlights the need to identify de novo molecular subtypes following CRT.

Despite substantial inter-tumor heterogeneity (FIG. 1C; FIGS. 5A-5C), recurrent gene expression programs were learned across malignant cells of different tumors by consensus non-negative matrix factorization (cNMF). cNMF was performed separately for the untreated and treated malignant cells and focused on the programs shared between patients that were biologically distinct (FIGS. 3A-3B; Tables 3,4; Methods)⁸⁶. Each program was annotated based on its top 200 weighted genes (Methods). In both untreated and treated tumors, nine malignant programs were identified that reflected either their lineage or cell state, though there was partial overlap among them (FIGS. 3A-3B).

TABLE 3

| | IFN | | IFN |
|---|---|---|---|
| Gene | signaling | Gene | signaling |
| LINC01496 | 0.0005166 | SPNS2 | 9.25E−05 |
| LYPD2 | 0.00031677 | CCDC60 | 9.22E−05 |
| MUC16 | 0.00027176 | EYA2 | 9.19E−05 |
| POU6F2 | 0.00022407 | GNA14 | 9.18E−05 |
| CDH4 | 0.00020897 | LY6E | 9.17E−05 |
| MUC4 | 0.00020748 | STK17B | 9.16E−05 |
| HECW2 | 0.00020594 | SP6 | 9.10E−05 |
| HDAC9 | 0.00020355 | MRVI1-AS1 | 9.09E−05 |
| MYO16- | 0.00017643 | HLA-A | 9.04E−05 |
| AS1 | | | |
| IFI44L | 0.0001763 | ARHGAP12 | 8.96E−05 |
| BCAT1 | 0.00017457 | DTX2 | 8.95E−05 |
| NCALD | 0.00016927 | B3GALT5 | 8.86E−05 |
| SLC15A2 | 0.00016783 | MX2 | 8.82E−05 |
| ALOX12- | 0.00016777 | FAM155A | 8.81E−05 |
| AS1 | | | |
| AC011306.1 | 0.00015842 | PROM2 | 8.78E−05 |
| IGSF1 | 0.0001551 | KRT13 | 8.73E−05 |
| GNGT1 | 0.00015248 | EPB41L4B | 8.73E−05 |
| ABCA12 | 0.00014645 | PTPRZ1 | 8.73E−05 |
| AC024084.1 | 0.00014621 | CASP10 | 8.72E−05 |
| KYNU | 0.00014401 | MAP3K9 | 8.68E−05 |
| SAMD9L | 0.00014159 | NR2F2-AS1 | 8.68E−05 |
| CLDN3 | 0.00014058 | ARFGAP3 | 8.62E−05 |
| CCDC146 | 0.00014012 | SP110 | 8.62E−05 |
| ASS1 | 0.00014006 | HLA-DRB5 | 8.61E−05 |
| LYN | 0.00013936 | NXPH1 | 8.52E−05 |
| DPPA5 | 0.00013856 | NCCRP1 | 8.50E−05 |
| ZBP1 | 0.00013607 | CD82 | 8.44E−05 |
| ZNF83 | 0.00013575 | MXD1 | 8.44E−05 |
| GABRB3 | 0.0001356 | C1GALT1 | 8.44E−05 |
| DOC2B | 0.00013543 | PPM1L | 8.43E−05 |
| POU6F2- | 0.00013481 | MYH14 | 8.43E−05 |
| AS2 | | | |
| IKZF2 | 0.00013255 | MBOAT1 | 8.41E−05 |
| ISG15 | 0.00013074 | IDO1 | 8.41E−05 |
| ADGRF1 | 0.00013054 | ALDH3B2 | 8.38E−05 |
| C17orf99 | 0.00012916 | STRIP2 | 8.38E−05 |
| KCNQ3 | 0.00012914 | APOL1 | 8.38E−05 |
| XAF1 | 0.00012818 | BCAS1 | 8.37E−05 |
| SLC15A1 | 0.00012705 | MUC13 | 8.34E−05 |
| GRHL1 | 0.00012704 | MIR3681HG | 8.33E−05 |
| VTCN1 | 0.00012673 | RHBDL2 | 8.29E−05 |
| SAMD9 | 0.00012667 | IGSF5 | 8.28E−05 |
| GPCPD1 | 0.00012608 | KRT4 | 8.28E−05 |
| VWA5B1 | 0.00012545 | SMPD3 | 8.22E−05 |
| CDH26 | 0.00012319 | SLC12A6 | 8.22E−05 |
| TTC9 | 0.00012308 | IFI27 | 8.16E−05 |
| APOL6 | 0.00012305 | C4orf19 | 8.15E−05 |
| AC026167.1 | 0.00012298 | HLA-B | 8.13E−05 |
| IFI44 | 0.00012253 | DDX60 | 8.11E−05 |
| TRIM22 | 0.00012122 | SREBF2 | 8.09E−05 |
| MACC1 | 0.00012024 | KRT23 | 8.09E−05 |
| EPSTI1 | 0.00011913 | TJP3 | 8.08E−05 |
| MBOAT2 | 0.00011824 | FNBP1 | 8.08E−05 |
| SLCO3A1 | 0.0001178 | SGPP2 | 8.03E−05 |
| NTN1 | 0.00011627 | PRRX2 | 7.99E−05 |
| ANKRD13A | 0.00011599 | VAV1 | 7.98E−05 |
| MPP7 | 0.00011527 | ERBB4 | 7.97E−05 |
| RARRES3 | 0.00011521 | PACSIN2 | 7.97E−05 |
| SOX5 | 0.00011343 | SPRR3 | 7.92E−05 |
| LY6D | 0.00011213 | CLDN4 | 7.92E−05 |
| ST3GAL4 | 0.00011167 | WARS | 7.91E−05 |
| B4GALT5 | 0.00011085 | TMEM106B | 7.89E−05 |
| GLI2 | 0.0001106 | ZNF185 | 7.89E−05 |
| SCEL | 0.00011043 | CFH | 7.85E−05 |
| LRMP | 0.00011037 | SRPX2 | 7.85E−05 |
| PLEKHG7 | 0.00010982 | STS | 7.85E−05 |
| RHOV | 0.00010927 | MUC5B | 7.82E−05 |
| BTNL2 | 0.0001088 | NCF4 | 7.81E−05 |
| NRP2 | 0.00010856 | FA2H | 7.79E−05 |
| NLRC5 | 0.00010667 | LINC01558 | 7.78E−05 |
| AGR3 | 0.00010655 | SYTL2 | 7.76E−05 |
| OAS1 | 0.00010607 | ISG20 | 7.75E−05 |
| CLIC5 | 0.00010549 | TMEM184A | 7.74E−05 |
| SLFN5 | 0.00010541 | AC019117.1 | 7.73E−05 |
| MAP3K13 | 0.00010494 | IFIT1 | 7.72E−05 |
| PARP14 | 0.00010478 | CBX6 | 7.72E−05 |
| PARP9 | 0.00010458 | EPS8L1 | 7.71E−05 |
| TNFAIP2 | 0.00010297 | BIK | 7.71E−05 |
| STAT1 | 0.00010209 | CHD3 | 7.71E−05 |
| RAB8B | 0.00010137 | MGAT4A | 7.69E−05 |
| SP140L | 0.00010135 | GNE | 7.67E−05 |
| TRIM31 | 0.00010104 | SWAP70 | 7.66E−05 |
| FBXO34 | 0.00010025 | DMGDH | 7.66E−05 |
| SLC44A5 | 9.99E−05 | GFPT1 | 7.65E−05 |
| PCLO | 9.92E−05 | IRF1 | 7.65E−05 |
| C2orf91 | 9.90E−05 | LINC01376 | 7.64E−05 |
| BIRC3 | 9.89E−05 | APBA1 | 7.63E−05 |
| PLCH1 | 9.81E−05 | RND3 | 7.62E−05 |
| CASC9 | 9.77E−05 | SORBS2 | 7.62E−05 |
| QPRT | 9.75E−05 | UNC5B | 7.61E−05 |
| PLCB1 | 9.70E−05 | ST14 | 7.60E−05 |
| C15orf48 | 9.66E−05 | DHRS9 | 7.58E−05 |
| GAN | 9.65E−05 | GALNT5 | 7.55E−05 |
| DOCK8 | 9.62E−05 | LIFR | 7.54E−05 |
| B2M | 9.61E−05 | SLC9A7 | 7.54E−05 |
| SP140 | 9.55E−05 | COL28A1 | 7.54E−05 |
| OAS3 | 9.51E−05 | CACNA2D3 | 7.53E−05 |
| CXCL17 | 9.47E−05 | NTN4 | 7.53E−05 |
| CEACAM1 | 9.44E−05 | EML4 | 7.53E−05 |
| A2ML1 | 9.42E−05 | | |
| NMUR2 | 9.38E−05 | | |
| TCF20 | 9.38E−05 | | |
| IFI6 | 9.35E−05 | | |

In untreated tumors, there were five lineage-specific programs: three spanned basal-like phenotypes involving the epithelial-mesenchymal transition^{87,88} (squamous, mesenchymal cytoskeletal, mesenchymal matrisomal), with the squamous program closely overlapping the basal-like A subtype⁷⁸; two spanned classical-like phenotypes (classicalprogenitor, classical activated) (FIG. 3A) with the classical progenitor program containing transcription factors (TFs) involved in endoderm lineage development (HNF1A, DLX2, PRDM6, and FOXO4) and the classical activated program also containing genes involved in secretion, cell polarization, and cytoskeletal remodeling. The remaining four cell state programs were cycling, hypoxic, TNF-NFkB signaling, and interferon signaling. In treated tumors, there were six lineage-specific programs (squamous, mesenchymal, induced basal-like, classical progenitor, classical acinar-like, classical neuroendocrine-like) and three cell state programs (cycling, TNF-NFkB signaling, interferon signaling) (FIG. 3B).

Programs varied in the extent to which they co-occurred within the same cell and associated with one another. In both untreated and treated tumors, IFN signaling and squamous program scores were correlated across nuclei (FIG. 12), as were those of mesenchymal with TNF-NFkB signaling. TNF-NFkB signaling was also correlated with the induced basal-like and classical neuroendocrine-like programs in the post-treatment context. Thus, basal-like cells may be more inflammatory/immunogenic overall than classical-like cells. Taken together, the increase in expression of basal-like genes, interferon signaling, and immune response promoting genes (FIG. 2C) in treated vs. untreated samples may reflect coupled programs in the same individual cells (FIG. 12), which could in turn drive immune cell state changes (FIGS. 9A-9B). One possibility is that these are driven through the effects of p63, especially the ΔNp63 isoform, though this remains an area of controversy in the field^{11,89,90}.

A Shift from Classical-Like to Induced Basal-Like or Terminally-Differentiated Pancreatic Cell Programs May Contribute to Resistance following Treatment

Compared to the untreated group, post-treatment malignant cells scoring highly for basal-like programs were enriched, while those scoring highly for classical-like programs were depleted (66% vs 19%; p0.0001, Fisher's exact test; FIG. 3C; FIG. 11). This was consistent with the differential expression analysis (FIG. 2C). Despite the overall reduction of classical-like cells post-treatment, the classical-like programs present in residual cells spanned a broader range of pancreatic cell lineages: classical progenitor (GATA6 TF), classical acinar-like (endoderm lineage TFs and characteristic digestive enzymes), and classical neuroendocrine-like (enriched for neural genes and target genes of pancreatic neuroendocrine TFs such as HNF3 and NKX6). This suggests that neoadjuvant CRT either selects for or drives cells towards a state of increased pancreatic differentiation (FIG. 3B) and mirrors the relative enrichment in non-neoplastic acinar and endocrine cells after CRT (FIG. 2A; FIGS. 8A-8B).

In addition, the post-treatment induced basal-like program shared features of both classical-like (classical activated) and basal-like (squamous) programs, along with MUC16 (also a member of the untreated interferon signaling program, FIG. 3A) and genes associated with chemoresistance (MUC13, CEACAM6, MUC5, FGF19, ABCC3, TNFAIP2, KLK11, DUSP1, OLFM4, AQP5)⁹¹⁻¹⁰¹ (FIG. 3B). This induced basal-like post-treatment program may reflect a phenotypic shift from the more therapeutically vulnerable classical-like subtype to the more resistant basal-like state reminiscent of recent ex vivo observations⁸¹.

More generally, these programs may reflect a shift towards either basal-like or terminally-differentiated pancreatic cell states that are advantageous for surviving CRT compared to a less differentiated classical-like phenotype (FIG. 3D). This model is consistent with in vitro and in vivo evidence for lineage plasticity in normal pancreatic ductal progenitor cells¹⁰² and differentiated pancreatic cells including a role for MYT1/MYT1L, a member of the classical neuroendocrine-like program, in ductal-neuroendocrine reprogramming (FIG. 3B; Tables 3,4)¹⁰³⁻¹⁰⁸.

TABLE 4

| | | |
|---|---|---|
| Topic | Gene ontology term | -Log(q-value) |

Untreated

| | | |
|---|---|---|
| Squamous | Charafe breast cancer luminal vs | 18.8 |
| | basal dn | |
| | Huper breast basal bs luminal up | 16.4 |
| | Hollern squamous breast tumor | 15.9 |
| | GO epidermis development | 12.2 |
| | GO epithelial cell differentiation | 12.0 |
| | GO epithelium development | 11.2 |
| | GO skin development | 11.1 |
| | GO keratinocyte development | 10.4 |
| | GO epidermal cell differentiation | 10.3 |
| | Smid breast cancer basal up | 10.1 |
| Mesenchymal | Module 47 (ECM and collagens) | 23.7 |
| cytoskeletal | Hallmark epithelial mesenchymal | 22.4 |
| | transition | |
| | Anastassiou multicancer | 22.1 |
| | invasiveness signature | |
| | Naba matrisome | 17.2 |
| | GO extracellular matrix | 15.9 |
| | GO locomotion | 15.5 |
| | GO supramolecular fiber | 15.5 |
| | organization | |
| | GO actin filament based process | 14.9 |
| | GO actin binding | 14.2 |
| | GO cell motility | 13.2 |
| Mesenchymal | Hallmark epithelial mesenchymal | 65.6 |
| matrisomal | transition | |
| | Anastassiou multicancer | 64.3 |
| | invasiveness signature | |
| | Module 47 (ECM and collagens) | 59.6 |
| | GO extracellular structure | 47.0 |
| | organization | |
| | GO collagen containing extracellular | 46.7 |
| | matrix | |
| | GO extracellular matrix | 42.5 |
| | NABA matrisome | 41.7 |
| | NABA core matrisome | 38.9 |
| | GO extracellular matrix structural | 37.2 |
| | constituent | |
| | Reactome extracellular matrix | 36.0 |
| | organization | |
| Classical Progenitor | Vecchi gastric cancer advanced vs | 19.4 |
| | early dn | |
| | PRDM6 target genes | 17.5 |
| | DLX2 target genes | 14.0 |
| | SMID breast cancer basal dn | 12.8 |
| | HNF1A target genes | 12.3 |
| | GO plasma membrane region | 10.3 |
| | GATA1 03 | 8.5 |
| | HNF1 01 | 7.5 |
| | Charafe breast cancer luminal vs | 6.9 |
| | mesenchymal up | |
| | GO ion transport | 4.6 |
| Classical activated | Dodd nasopharyngeal carcinoma up | 13.2 |
| | GO cell leading edge | 5.8 |
| | GO apical part of cell | 4.5 |
| | GO secretory vesicle | 4.2 |
| | GO cell projection membrane | 3.9 |
| | GO secretion | 3.9 |
| | GO cytoskeletal protein binding | 3.9 |
| | HNF1A target genes | 3.6 |
| | GO actin binding | 3.0 |
| | Smid breast cancer basal dn | 2.9 |
| Cycling | Fischer dream targets | 285.9 |
| | GO cell cycle | 139.1 |
| | Benporath cycling genes | 126.6 |
| | GO mitotic cell cycle | 123.3 |
| | GO cell cycle process | 122.3 |
| | Reactome cell cycle | 105.1 |
| Hypoxic | Hallmark hypoxia | 50.5 |
| | Mense hypoxia up | 49.1 |
| | Nakamura tumor zone | 47.7 |
| | Elvidge hypoxia up | 46.9 |
| | Winter hypoxia metagene | 42.6 |
| | Hallmark glycolysis | 21.8 |
| TNF-NFkB | Zhang response to IKK inhibitor and | 43.0 |
| signaling | TNF up | |
| | Hallmark TNFA signaling via | 32.0 |
| | NFkbB | |
| | Phong TNF response not via p38 | 27.5 |
| | Phong TNF targets up | 22.8 |
| | Charafe breast cancer luminal vs | 21.4 |
| | basal dn | |
| | Phong TNF response via p38 partial | 20.7 |
| | Hinata NFKB targets keratinocyte up | 18.7 |
| | GO epithelium development | 17.9 |
| | Tian TNF signaling via NFKB | 15.6 |
| | GO cytokine mediated signaling | 14.9 |
| | pathway | |
| IFN signaling | Hallmark interferon gamma response | 25.7 |
| | Sana response to IFNG up | 20.9 |
| | Hecker IFNB 1 targets | 19.3 |
| | Hallmark interferon alpha response | 17.5 |
| | Browne interferon responsive genes | 16.9 |
| | Zhang interferon response | 13.8 |
| | Reactome interferon alpha beta | 13.2 |
| | signaling | |
| | Reactome interferon signaling | 12.3 |
| | GO response to type I interferon | 11.5 |
| | Einav interferon signature in cancer | 11.3 |

Treated

| | | |
|---|---|---|
| Squamous | Koniuma targets of SMAD2 or | 11.5 |
| | SMAD3 | |
| | Pece mammary stem cell up | 11.5 |
| | Wu cell migration | 8.8 |
| | Enk UV response keratinocyte up | 8.4 |
| | Huper breast basal vs luminal up | 5.6 |
| | GO epithelium development | 5.3 |
| | GO epithelial cell differentiation | 4.2 |
| Mesenchymal | Charafe breast cancer luminal vs | 11.9 |
| | basal dn | |
| | Vecchi gastric cancer advanced vs | 11.4 |
| | early up | |
| | Charafe breast cancer luminal vs | 10.9 |
| | mesenchymal dn | |
| | Nakamura tumor zone peripheral vs | 8.6 |
| | central up | |
| | Hallmark epithelial mesenchymal | 8.6 |
| | transition | |
| | Reactome extracellular matrix | 8.3 |
| | organization | |
| | Module 47 (ECM and collagens) | 8.0 |
| | Wu cell migration | 8.0 |
| | GO extracellular structure | 7.1 |
| | organization | |
| | GO extracellular matrix | 3.8 |
| Induced basal-like | Dodd nasopharyngeal carcinoma up | 18.5 |
| | Wu cell migration | 7.8 |
| | Charafe breast cancer basal vs | 7.3 |
| | mesenchymal up | |
| | Bosco epithelial differentiation | 5.8 |
| | module | |
| | HFNA1A target genes | 5.3 |
| | Reactome development biology | 5.0 |
| | Smid breast cancer basal up | 4.6 |
| | Enk UV response keratinocyte up | 4.4 |
| | GO response to drug | 3.8 |
| | GO response to organic cyclic | 3.8 |
| | compound | |
| Classical progenitor | Vecchi gastric cancer advanced vs | 18.5 |
| | early dn | |
| | Wu cell migration | 7.8 |
| | Charafe breast cancer basal vs | 7.3 |
| | mesenchymal up | |
| | Bosco epithelial differentiation | 5.8 |
| | module | |
| | HFNA1A target genes | 5.3 |
| | Dodd nasopharyngeal carcinoma up | 3.3 |
| | Smid breast cancer basal dn | 2.9 |
| | DLX2 target genes | 2.4 |
| | HNF1A target genes | 2.3 |
| | Smid breast cancer luminal b up | 2.3 |
| | GO apical part of cell | 2.2 |
| Classical acinar-like | GNF2 SPINK 1 | 28.1 |
| | GNF2 SERPINI2 | 19.3 |
| | GO digestion | 8.7 |
| | Smid breast cancer basal dn | 5.0 |
| | Reactome digestion of dietary lipid | 4.3 |
| | GO digestive system process | 4.1 |
| | GO proteolysis | 3.8 |
| | GATA6 01 | 3.7 |
| | GO peptidase activity | 3.1 |
| | Reactome digestion | 2.4 |
| Classical | GO synapse | 13.8 |
| neuroendocrine-like | Reactome neuronal system | 11.2 |
| | GO synaptic signaling | 9.1 |
| | GO neuron differentiation | 7.4 |
| | GO vesicle mediated transport in | 7.3 |
| | synapse | |
| | GO neuron development | 6.7 |
| | Reactome regulation of insulin | |
| | secretion | |
| Cycling | Fischer dream targets | 220.7 |
| | GO cell cycle | 111.6 |
| | Benoporath cycling genes | 110.4 |
| | GO mitotic cell cycle | 101.8 |
| | GO cell cycle process | 94.1 |
| | Reactome cell cycle | 85.0 |
| | GO cell division | 73.1 |
| | Hallmark E2F targets | 72.9 |
| TNF-NFkB | Zwang clas 3 transiently induced by | 22.3 |
| signaling | EGF | |
| | Charafe breast cancer luminal vs | 13.9 |
| | basal dn | |
| | GO locomotion | 12.5 |
| | Phong TNF response not via p38 | 12.5 |
| | GO cell motility | 10.9 |
| | Zhang response to IKK inhibitor and | 9.0 |
| | TNF up | |
| | Hallmark TNFA signaling via NFKB | 8.6 |
| | GO regulation of epithelial cell | 5.1 |
| | migration | |
| | GO taxis | 5.0 |
| | Smid breast cancer basal up | 4.9 |
| IFN signaling | Reactome interferon signaling | 7.3 |
| | Hallmark interferon alpha response | 6.9 |
| | Reactome innate immune system | 6.6 |
| | Reactome interferon alpha beta | 5.4 |
| | signaling | |
| | Hallmark interferon gamma response | 5.3 |
| | Reactome antigen processing cross | 4.5 |
| | presentation | |
| | Reactome AP folding assembly and | 3.6 |
| | peptide loading of clas I MHC | |
| | Reactome cytokine signaling in | 3.6 |
| | immune system | |
| | Reactome interferon signaling | 7.3 |
| | Hallmark interferon alpha response | 6.9 |

TABLE 5

| | | |
|---|---|---|
| Topic | Gene ontology term | -Log(q-value) |

Untreated

| | | |
|---|---|---|
| Myofibroblast | Hallmark epithelial | 35.2 |
| | mesenchymal transition | |
| | GO extracellular matrix | 26.5 |
| | GO biological adhesion | 24.4 |
| | GO collagen containing | 24.3 |
| | extracellular matrix | |
| | GO locomotion | 24.0 |
| | Naba matrisome | 20.7 |
| | GO cell motility | 20.4 |
| | GO actin cytoskeleton | 18.1 |
| Secretory | Reactome eukaryotic | 86.4 |
| | translation elongation | |
| | Kegg ribosome | 81.1 |
| | GO SRP dependent | 78.0 |
| | cotranslational protein | |
| | targeting to membrane | |
| | GO establishment of protein | 74.9 |
| | localization to endoplasmic | |
| | reticulum | |
| | GO translational initiation | 63.9 |
| | GO protein targeting to | 62.1 |
| | memebrane | |
| | GO secretory granule | 17.5 |
| | GO secretory vesicle | 16.1 |
| | GO exocytosis | 14.7 |
| | GO cytokine mediated | 11.7 |
| | signaling pathway | |
| Neurotropic | GO neurogenesis | 13.9 |
| | GO neuron differentiation | 13.4 |
| | GO neuron development | 12.1 |
| | GO synapse | 9.6 |
| | GO synapse organization | 9.4 |
| | GO postsynapse | 7.4 |
| Mesodermal progenitor | PRDM6 target genes | 24.1 |
| | Lim mammary stem cell up | 17.3 |
| | GO embryo development | 16.7 |
| | Boquest stem cell up | 15.2 |
| | GO skeletal system | 1.4 |
| | development | |
| | GO circulatory system | 12.7 |
| | development | |
| | GO cardiovascular system | 11.1 |
| | development | |
| | GO cartilage development | 10.0 |
| | GO connective tissue | 9.4 |
| | development | |
| | GO mesenchyme | 7.4 |
| | development | |

Treated

| | | |
|---|---|---|
| Myofibroblast | Hallmark epithelial | 24.3 |
| | mesenchymal transition | |
| | GO biological adhesion | 18.8 |
| | GO locomotion | 18.8 |
| | GO cell motility | 18.6 |
| | GO extracellular structure | 16.9 |
| | organization | |
| | Naba matrisome | 16.2 |
| | GO cell adhesion molecule | 15.0 |
| | binding | |
| | GO extracellular matrix | 14.5 |
| | GO collagen containing | 13.9 |
| | extracellular matrix | |
| | Go response to wounding | 3.4 |
| Secretory | Reactome eukaryotic | 125.7 |
| | transition elongation | |
| | GO cotranslational protein | 115.8 |
| | targeting to membrane | |
| | GO establishment of protein | 111 |
| | localization to ER | |
| | GO protein targeting to | 92.6 |
| | membrane | |
| | GO peptide biosynthetic | 65.4 |
| | process | |
| | GO secretory vesicle | 12.4 |
| | GO exocytosis | 12.2 |
| | GO secretory granule | 12.1 |
| | GO response to cytokine | 10.6 |
| | GO secretion | 7.7 |
| Neurotropic | GO neurogenesis | 14.9 |
| | GO neuron differentiation | 12.9 |
| | GO neuron development | 10.1 |
| | GO cell projection | 9.4 |
| | organization | |
| | GO postsynapse | 7.6 |
| Neuromuscular | GO actin filament based | 16.5 |
| | process | |
| | GO muscle system process | 16.1 |
| | GO muscle structure | 14.4 |
| | development | |
| | GO contractile fiber | 13.5 |
| | GO muscle contraction | 13.4 |
| | GO I band | 12.1 |
| | GO action potential | 11.1 |
| | GO regulation of membrane | 11.0 |
| | potential | |
| | GO synapse | 10.4 |
| | GO muscle cell | 10.1 |
| | differentiation | |

### Treatment-Associated Contractile Phenotype in Cancer-Associated Fibroblasts

Because fibroblasts have emerged as a key instigator of tumor-immune evasion and therapeutic target¹⁰⁹, the successful capture of fibroblasts were leveraged in the data to assess their subsets, programs, and response to treatment. Although three signatures were recently reported in a scRNA-seq study of human and murine PDAC (myofibroblastic CAFs (myCAF), inflammatory CAFs (iCAF), and antigen-presenting CAFs (apCAF))si, these did not adequately segregatethe snRNA-seq data, likely reflecting the substantial underrepresentation of CAFs in earlier scRNA-seq studies⁵¹ (FIG. 14). Thus, cNMF was separately applied to learn four snRNA-seq programs each for the untreated and treated CAFs (FIG. 15B; Table 5; Methods).

Three of the programs were shared between untreated and treated CAFs: myofibroblast, neurotropic, and secretory, while a mesodermal progenitor program (mesodermal developmental genes and TFs) was discovered only in untreated CAFs, and a neuromuscular program (muscle development, contractility, synapse, and action potential genes) was only learned in treated CAFs (FIG. 15B). The myofibroblast program included extracellular matrix, motility, and wound response genes; the neurotropic program featured neural development, synaptic, and axonal guidance genes; and the secretory program encompassed protein targeting, secretory vesicle, exocytosis, and cytokine signaling genes including modulators of diverse immune functions (e.g., CXCL14, LGALS1, CST3, PPIA, LGALS3BP, CD59, CD81, and IFITM3)¹¹⁰⁻¹¹⁵. In untreated CAFs, the myofibroblast and mesodermal progenitor programs were positively correlated (FIG. 15C).

The marked enrichment of the myofibroblast phenotype after neoadjuvant CRT (FIG. 15D) may be consistent with an induction of genes associated with muscle development and contraction after CRT seen in differential expression analysis (FIG. 15A). BCL2, an anti-apoptotic protein, was also expressed at a higher level in post-CRT myofibroblasts (FIG. 15A). BCL2 inhibition with Navitoclax (ABT-263) induced apoptosis of myofibroblasts and impeded tumor growth in a xenograft mouse model of hepatocellular cancer¹¹⁶, and could thus be a relevant therapeutic strategy in the neoadjuvant setting. Conversely, the expression of FAP, a member of the mesodermal progenitor program, was significantly reduced after CRT, suggesting that depletion of CAFs by blocking FAP may not confer added value, despite prior studies showing promising anti-tumor effects in some model systems^{27,117} (FIG. 15A). Treatment was also associated with higher CXCL12 expression in CAFs (FIG. 15A) and increased predicted CXCL12-CXCR4 interactions between CAFs and CD8⁺ T-cells (FIG. 2B), which has been linked to inhibition of T-cell migration to the TME¹¹⁸. Moreover, CXCR4 inhibition improves PDAC sensitivity to anti-PD-1/PD-L1 immunotherapy^{119,120}. Thus, modulating this axis may further improve clinical outcomes associated with neoadjuvant CRT.

### Clinically Relevant Molecular Taxonomy Based on Malignant and Fibroblast Programs Improves Prognostication

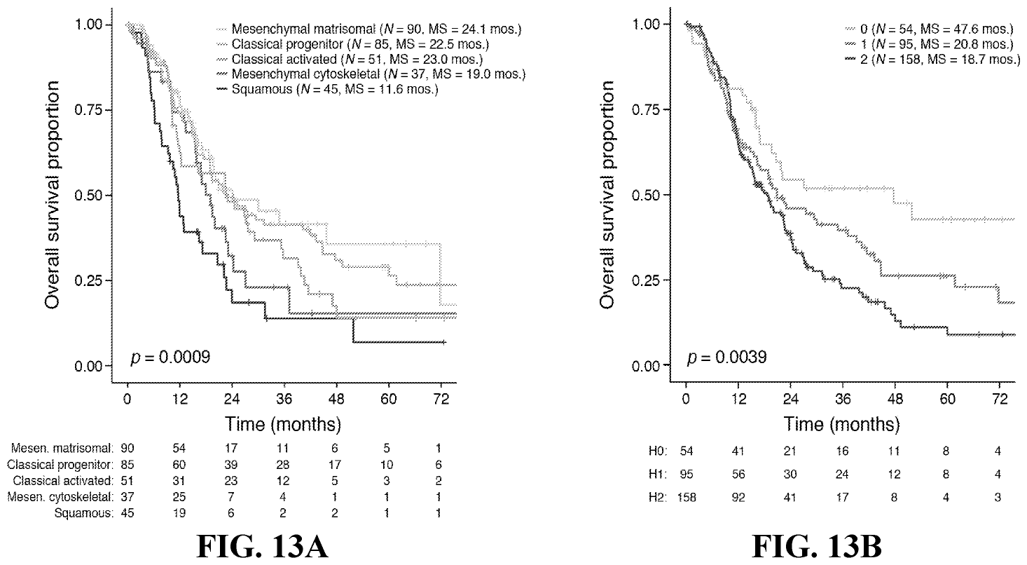

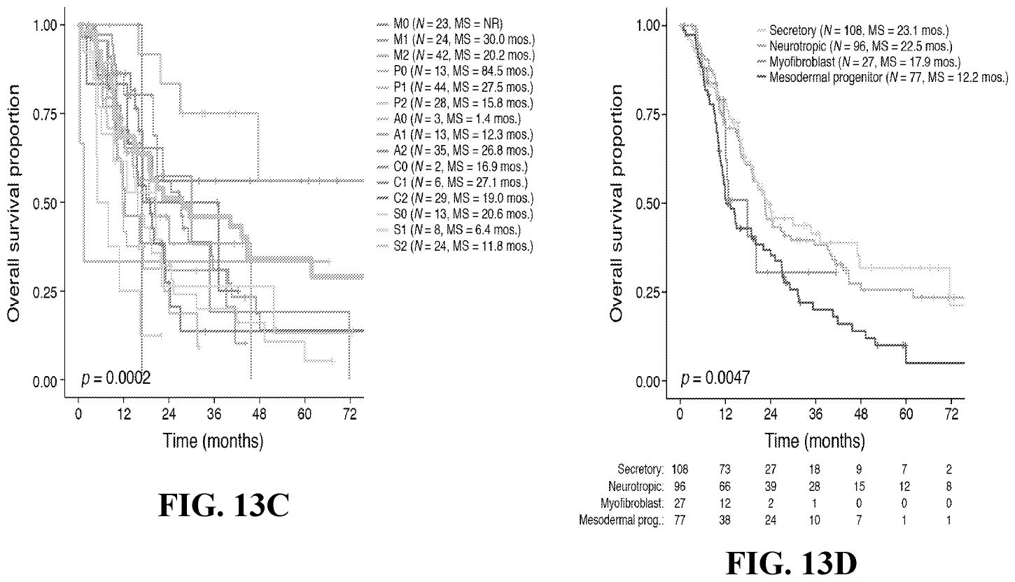

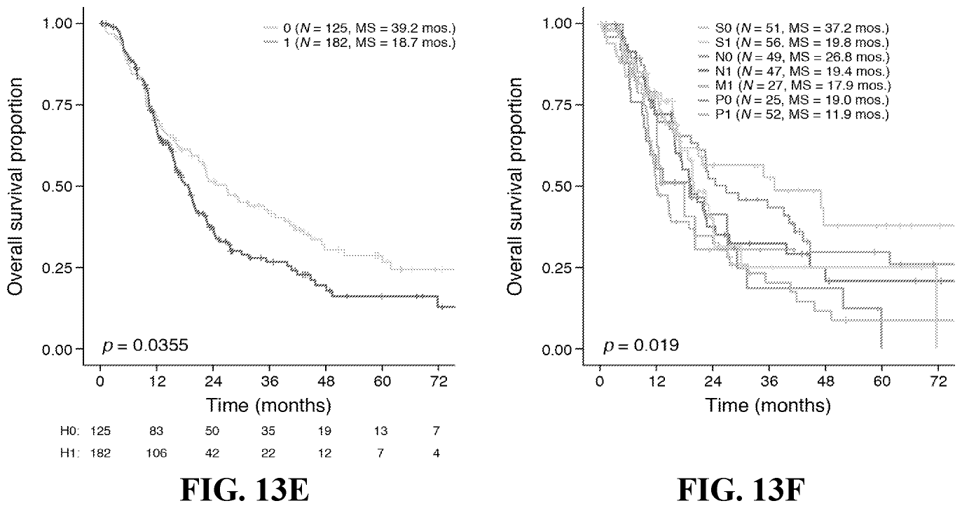

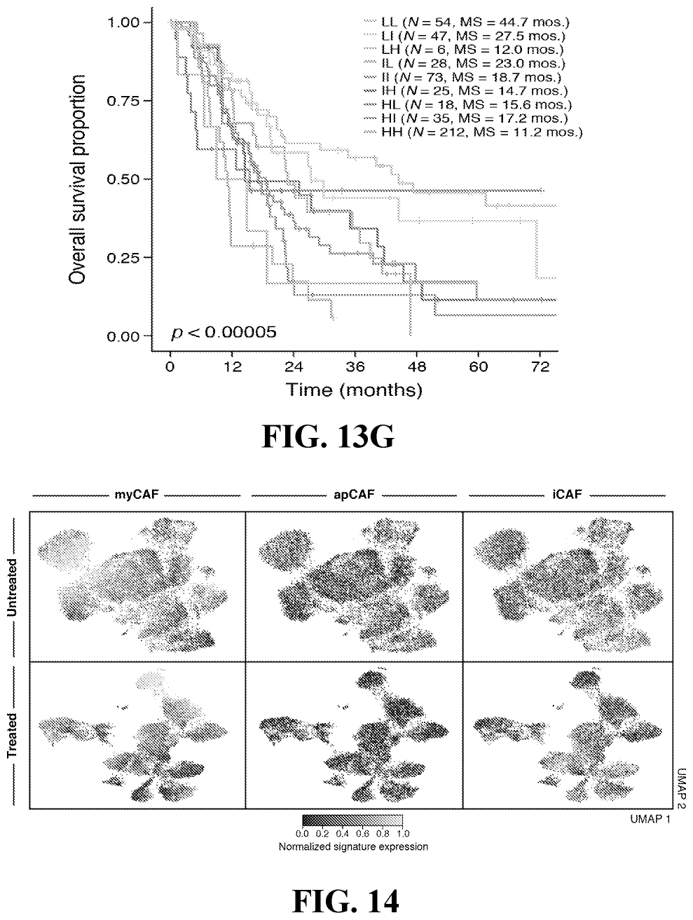

Prior survival analyses that stratified patients by bulk expression subtypes only discerned binary prognostic differences between basal-like and classical-like tumors^{7,10}, but finer subsets, such as three non-basal bulk subtypes¹⁰ (pancreatic progenitor, immunogenic, ADEX) were indistinguishable. The de novo snRNA-seq programs were used for untreated malignant cells and fibroblasts to stratify bulk RNA-seq profiles from untreated, resected primary PDAC in the TCGA¹¹ and PanCuRx⁷⁸ cohorts (n=307; Methods). To account for the effects of different cell types on survival, both malignant and fibroblast untreated programs were considered, separately and in combination, to assign patients to risk categories (Methods). Briefly, each untreated tumor was scored by the five lineage malignant programs and four fibroblast programs. Each tumor was characterized by (1) its top scoring program (primary program) and (2) the number of highly expressed programs (heterogeneity score) and assigned by these two criteria to one of 15 possible malignant classes (FIG. 3E, rows) and 8 possible fibroblast classes (FIG. 3E; columns). Next, the survival curves associated with each class were inspected (FIGS. 13A-13F) and aggregated them into three putative risk categories (separately for malignant or fibroblast): low, intermediate, and high (FIG. 3E, red and blue color bars). Finally, tumors were also assigned to four combined risk strata by integrating the malignant and fibroblast risk groups: low (both low), high (both high), high-intermediate (one high, one intermediate), low-intermediate (all others) (FIG. 3E, internal grid, purple color code).

Kaplan-Meier (KM) analyses of overall survival (OS) stratified by either primary program, heterogeneity score, or both were separately prognostic for malignant cells and fibroblasts (FIGS. 13A-13F). Among the malignant programs, mesenchymal matrisomal and classicalprogenitor were associated with the best OS, classical activated was associated with intermediate OS, and squamous and mesenchymal cytoskeletal were associated with the worst OS (FIG. 13A). Notably, the basal-like mesenchymal matrisomal program had survival outcomes comparable to classicalprogenitor. Moreover, the classical activated program was identified as a classical-like subset with worse outcomes than the classicalprogenitor program. Among fibroblast programs, the secretory and neurotropic programs were associated with longer survival while the myofibroblast and mesodermal progenitor programs were associated with shorter survival (FIG. 13D). These findings were consistent with prior work associating myofibroblasts and mesenchymal stem-like cells with poor prognosis in a range of solid cancers¹²¹⁻¹²³. For both malignant cells and CAFs, an increasing number of highly scoring programs in one tumor (heterogeneity score) was associated with worse OS (FIGS. 13B and 13E), consistent with the known association between intratumoral heterogeneity, treatment resistance, and poor outcomes^{29,124}. When combining the malignant and fibroblast risk groups into a four-tier risk stratification (FIG. 3E), there was a significant prognostic difference among the combined strata (log-rank p<0.00005; FIG. 311) with a greater dynamic range (median survival: 11.2 to 44.7 months) than seen with prior classifications¹⁰.

### Digital Spatial Profiling Reveals Malignant- and Fibroblast-Intrinsic Programs May Modulate Local Immune Microniches

Our snRNA-seq analysis highlighted multiple potential inter-compartmental interactions among malignant, stromal and immune cells, including those associated with CRT. Identifying cancer cell- and fibroblast-intrinsic programs that may govern local immune microniches remains an open question in PDAC research, with prior studies disagreeing on whether the basal-like or classical-like subtype is correlated with immune exclusion^{11,89,90}. Moreover, elucidating the relationships between the newly-identified neoplastic, lineage-specific programs^{81,102-108} and local immune infiltration will be critical in guiding therapeutic development.

To address this challenge, digital spatial profiling (DSP) was performed with the GeoMx platform (NanoString) and a cancer transcriptome atlas (CTA) probe set. In this method, UV-photocleavable barcode-conjugated RNA ISH probes against 1,412 target mRNAs were used to capture and profile mRNA from user-defined regions of interest (ROI) (FIG. 16A; Methods)¹²⁵. A four-color immunofluorescence slide scan for each specimen (FIG. 4A) showed intra-tumoral diversity in tissue architecture, allowing us to profile three distinct classes of ROIs encompassing neoplastic cells with either (1) immune and CAF infiltration, (2) immune infiltration only, or (3) CAF infiltration only (FIG. 4B). Custom illumination masks were then used to separately capture RNA from areas of illumination (AOI) enriched for one cell type within the ROI, collected RNA ISH barcodes from each AOI in a spatially-indexed manner, and counted transcripts by sequencing (FIG. 16A; Methods).

The snRNA-seq cell type signatures were used to deconvolve the spatial profiles. The malignant, CAF, and immune AOIs clustered appropriately by cell type, demonstrating the coherence and complementarity of the two platforms (FIG. 16B). The expression of each malignant was also mapped and CAF program onto the spatial data (FIG. 3A, 3B; FIG. 15B). Consistent with the snRNA-seq results, within the malignant compartment, CRT was associated with a higher basal-like score (mixed effect model p=0.0035) but not classical-like score (mixed effect model p>0.2) compared to untreated specimens (FIG. 4C). Because only 269 genes were shared between the 1,412 gene CTA panel and the 2,037 program genes, and also profiled three untreated specimens using a whole transcriptome atlas (WTA; 18,269 genes; one probe per gene; 1,918 of the program genes) (FIG. 4D; Methods). Programs from untreated patients had concordant expression within the WTA and CTA data (Spearman's ρ=0.54-0.999; FIG. 16E) and mapped to locally-defined architecture within the tumor in a comparable manner (FIG. 4D).

Relating the expression of each malignant and CAF program (FIG. 3A-3B; FIG. 15B) to the physical architecture of the tumor (Methods), the CAF myofibroblast program was associated with immune exclusion in treated samples (mixed effect model p=0.021), consistent with the role of myofibroblasts in mediating an immunosuppressive TME¹²⁶ (FIG. 15B). Immune-excluded ROIs (class 3) were also associated with higher untreated classical activated malignant program expression (mixed effect model p=0.020), while the untreated mesenchymal matrisomal malignant program was associated with immune infiltration (mixed effect model p=0.0035) (Table 6A-6C. The association between the classical activated program and immune exclusion may be related to the poorer prognosis ound for this program in untreated patients compared to other classical-like programs (FIGS. 3E-3F). Similarly, the association between the mesenchymal matrisomal program and immune infiltration may be related to the longer survival found for this program in untreated patients compared to other basal-like programs (FIGS. 3E-3F). In post-treatment tumors, the classical neuroendocrine-like, squamous, and induced basal-like malignant programs associated with or trended towards an association with immune-infiltrated ROIs (mixed effect p=0.029, 0.074, 0.091, respectively). These spatially resolved findings support an overall association between basal-like programs and immune infiltration and conversely, between specific classical-like programs and immune exclusion with certain exceptions. Despite these overall trends, some ROIs that contained high classical scoring epithelial segments also featured an immune infiltrate, which warranted further exploration.

TABLE 6A
Untreated Malignant Topics

| Topic | Test | AOI_type | Treatment_status | Pval | Directionality |
|---|---|---|---|---|---|
| Cycling | Immune infiltration | Epithelial | Untreated | 0.802486 | |
| Mesenchymal | Immune infiltration | Epithelial | Untreated | 0.361175 | |
| cytoskeletal | | | | | |
| Squamous | Immune infiltration | Epithelial | Untreated | 0.421951 | |
| Classical | Immune infiltration | Epithelial | Untreated | 0.871704 | |
| progenitor | | | | | |
| Classical | Immune infiltration | Epithelial | Untreated | 0.020290 | Higher in |
| activated | | | | | excluded |
| Mesenchymal | Immune infiltration | Epithelial | Untreated | 0.003452 | Higher in |
| matrisomal | | | | | infiltrated |
| TNF-NFKB | Immune infiltration | Epithelial | Untreated | 0.596529 | |
| signaling | | | | | |
| IFN signaling | Immune infiltration | Epithelial | Untreated | 0.226591 | |
| Hypoxic | Immune infiltration | Epithelial | Untreated | 0.073327 | Higher in |
| | | | | | excluded |
| Cycling | Fibroblast presence | Epithelial | Untreated | 0.016917 | Higher with |
| | | | | | fibroblasts |
| Mesenchymal | Fibroblast presence | Epithelial | Untreated | 0.000317 | Higher with |
| cytoskeletal | | | | | fibroblasts |
| Squamous | Fibroblast presence | Epithelial | Untreated | 0.285271 | |
| Classical | Fibroblast presence | Epithelial | Untreated | 0.733061 | |
| progenitor | | | | | |
| Classical | Fibroblast presence | Epithelial | Untreated | 0.853558 | |
| activated | | | | | |
| \Mesenchymal | Fibroblast presence | Epithelial | Untreated | 0.317429 | |
| matrisomal | | | | | |
| TNF-NFKB | Fibroblast presence | Epithelial | Untreated | 0.006850 | Higher with |
| signaling | | | | | fibroblasts |
| IFN signaling | Fibroblast presence | Epithelial | Untreated | 0.203898 | |
| Hypoxic | Fibroblast presence | Epithelial | Untreated | 0.017399 | Higher with |
| | | | | | fibroblasts |

TABLE 6B
Treated malignant topics

| Topic | Test | AOI_type | Treatment_status | Pval | Directionality |
|---|---|---|---|---|---|
| Squamous | Immune | Epithelial | Treated | 0.073620 | Higher in |
| | infiltration | | | | excluded |
| TNF-NFKB | Immune | Epithelial | Treated | 0.070746 | Higher in |
| signaling | infiltration | | | | excluded |
| Mesenchymal | Immune | Epithelial | Treated | 0.253188 | |
| | infiltration | | | | |
| Cycling | Immune | Epithelial | Treated | 0.928114 | |
| | infiltration | | | | |
| Induced basal- | Immune | Epithelial | Treated | 0.091379 | Higher in |
| like | infiltration | | | | infiltrated |
| Classical | Immune | Epithelial | Treated | 0.028733 | Higher in |
| neuroendocrine- | infiltration | | | | infiltrated |
| like | | | | | |
| Classical | Immune | Epithelial | Treated | 0.740882 | |
| progenitor | infiltration | | | | |
| Classical acinar- | Immune | Epithelial | Treated | 0.126878 | |
| Like | infiltration | | | | |
| IFN signaling | Immune | Epithelial | Treated | 0.686557 | |
| | infiltration | | | | |
| Squamous | Fibroblast | Epithelial | Treated | 0.015349 | Higher with |
| | presence | | | | fibroblasts |
| TNF-NFkB | Fibroblast | Epithelial | Treated | 0.138150 | |
| Signaling | presence | | | | |
| Mesenchymal | Fibroblast | Epithelial | Treated | 0.007410 | Higher with |
| | presence | | | | fibroblasts |
| Cycling | Fibroblast | Epithelial | Treated | 0.854280 | |
| | presence | | | | |
| Induced basal- | Fibroblast | Epithelial | Treated | 0.546008 | |
| like | presence | | | | |
| Classical | Fibroblast | Epithelial | Treated | 0.976679 | |
| neuroendocrine- | presence | | | | |
| like | | | | | |
| Classical | Fibroblast | Epithelial | Treated | 0.016719 | Higher |
| progenitor | presence | | | | without |
| | | | | | fibroblasts |
| Classical acinar- | Fibroblast | Epithelial | Treated | 0.496951 | |
| Like | presence | | | | |
| IFN signaling | Fibroblast | Epithelial | Treated | 0.346570 | |
| | presence | | | | |

TABLE 6C
Fibroblast Topics

| Topic | Test | AOI_type | Treatment_status | Pval | Directionality |
|---|---|---|---|---|---|
| Myofibroblast | Immune infiltration | Fibroblast | Treated | 0.020630 | Higher in |
| | | | | | excluded |
| Secretory | Immune infiltration | Fibroblast | Treated | 0.113055 | |
| Neuromuscular | Immune infiltration | Fibroblast | Treated | 0.271436 | |
| Neurotropic | Immune infiltration | Fibroblast | Treated | 0.282765 | |
| Myofibroblast | Immune infiltration | Fibroblast | Untreated | 0.310751 | |
| Neurotropic | Immune infiltration | Fibroblast | Untreated | 0.444209 | |
| Secretory | Immune infiltration | Fibroblast | Untreated | 0.927667 | |
| Mesodermal | Immune infiltration | Fibroblast | Untreated | 0.592326 | |
| progenitor | | | | | |

There were also differences in the types of immune infiltrates surrounding basal-like or classical-like malignant cells. The association between immune infiltrates with malignant cell subtype was observed by unsupervised clustering of immune cell type-specific and functional module genes measured within immune AOIs (p=0.0339, χ² test; FIG. 4E; Methods). Moreover, immune AOIs in classical-like segments had higher expression of clusters of macrophage-rich genes (p=0.0341, t-test), while those from basal-like segments had higher expression of clusters of T lymphocyte, B lymphocyte and dendritic cell genes (and depleted of macrophage genes) (p=6.74×10⁻⁵ and 0.047, t-test, for macrophage-depleted gene clusters A and B, respectively, FIG. 4E; FIG. 16C). Moreover, at the individual gene level, epithelial AOI subtype (classical-like vs basal-like) was associated with expression of distinct immune lineage-restricted and modulatory genes (FIG. 4F; Table 7): basal-like segments had immune AOIs with higher expression of IFNG-induced chemokines (CXCL9, CXCL10, CXCL11), markers of cytotoxic T lymphocytes and NK cells (CD8A, CD3E, CD247, GZMA, GZMB, GZMK, PRF1, NKG7, NCAM1), immune checkpoints/markers of cytotoxic T lymphocyte exhaustion (PDCD1, CD274, LAG3, ENTPD1, TIGIT, IDO1), and markers of regulatory T cells (NT5E, FOXP3) (FIG. 4F). In contrast, classical-like segments had immune AOIs with higher expression of MHC-II (HLA-DQA1/2, HLA-DOA, HLA-DMA, HLA-DPA1) and macrophage markers (CD68, CD163, CSFIR). Taken together, these analyses suggest distinct immune niches associated with basal-like and classical-like malignant cells.

TABLE 7
Subtype Associated Immune Genes

| | Log2 | | |
|---|---|---|---|
| | Fold | | FDR |
| Gene | change | Pval | (BH) |
| ADA | 0.451913 | 0.001079 | 0.013903 |
| ALCAM | −0.50628 | 0.001479 | 0.01806 |
| APOL6 | 0.157205 | 0.108611 | 0.227007 |
| ARID1A | −0.24179 | 0.012571 | 0.056862 |
| ARID1B | −0.26098 | 0.022132 | 0.072319 |
| ARID2 | 0.063986 | 0.543766 | 0.643641 |
| ATG5 | 0.178618 | 0.073762 | 0.169434 |
| ATG7 | −0.17932 | 0.07268 | 0.168874 |
| ATR | 0.16948 | 0.09545 | 0.208908 |
| ATRX | 0.085582 | 0.451097 | 0.568775 |
| BCAT1 | 0.344332 | 0.019267 | 0.072098 |
| BCL6 | −0.08351 | 0.33211 | 0.452504 |
| BMP6 | 0.291265 | 0.013738 | 0.057948 |
| C1QA | −0.38063 | 0.236802 | 0.366254 |
| C1QB | −0.38555 | 0.284927 | 0.413145 |
| C2 | −0.27738 | 0.153288 | 0.284503 |
| C3AR1 | −0.17013 | 0.231605 | 0.366254 |
| C5AR1 | −0.03762 | 0.823437 | 0.858596 |
| CACNA2D3 | 0.189741 | 0.063451 | 0.154954 |
| CASP10 | 0.332997 | 0.016936 | 0.067744 |
| CASP3 | 0.147925 | 0.261926 | 0.395569 |
| CCR6 | 0.17796 | 0.121491 | 0.240905 |
| CD14 | −0.11321 | 0.608083 | 0.678296 |
| CD163 | −0.2497 | 0.423283 | 0.54255 |
| CD19 | 0.397377 | 0.00092 | 0.013333 |
| CD1A | 0.077483 | 0.608128 | 0.678296 |
| CD1C | 0.337134 | 0.003115 | 0.025814 |
| CD200 | 0.235399 | 0.03441 | 0.096183 |
| CD247 | 0.131999 | 0.333512 | 0.452504 |
| CD27 | 0.52889 | 0.004942 | 0.033721 |
| CD274 | 0.276525 | 0.000587 | 0.010484 |
| CD300A | 0.128735 | 0.203245 | 0.336471 |
| CD3E | 0.109561 | 0.393887 | 0.523955 |
| CD4 | −0.45253 | 0.01732 | 0.068104 |
| CD40 | 0.15072 | 0.084025 | 0.189261 |
| CD40LG | 0.227025 | 0.004816 | 0.033721 |
| CD47 | −0.00282 | 0.9706 | 0.9706 |
| CD5 | 0.19247 | 0.068194 | 0.163102 |
| CD53 | −0.39266 | 0.021421 | 0.072319 |
| CD68 | −0.90783 | 0.00909 | 0.049058 |
| CD7 | 0.113288 | 0.371466 | 0.49815 |
| CD79A | 0.806861 | 0.015904 | 0.064731 |
| CD79B | 0.384774 | 0.002186 | 0.02205 |
| CD81 | −0.94915 | 0.000376 | 0.009276 |
| CD84 | −0.07112 | 0.60347 | 0.678296 |
| CD8A | 0.271582 | 0.021666 | 0.072319 |
| CDC25B | 0.271579 | 0.008998 | 0.049058 |
| CDC7 | 0.285325 | 0.01311 | 0.056862 |
| CDKN1A | −0.32992 | 0.008781 | 0.049058 |
| CDKN1B | 0.163549 | 0.396215 | 0.523955 |
| CLEC4C | 0.184507 | 0.199715 | 0.335752 |
| CLEC5A | 0.254089 | 0.141155 | 0.270644 |
| CLEC7A | −0.18187 | 0.202943 | 0.336471 |
| CLU | −0.0199 | 0.849562 | 0.875993 |
| CMKLR1 | −0.01275 | 0.925637 | 0.937763 |
| COLEC12 | 0.011364 | 0.946499 | 0.95473 |
| CPA3 | −0.5522 | 0.01245 | 0.056862 |
| CR2 | 0.403944 | 0.021842 | 0.072319 |
| CRLF2 | 0.292287 | 0.021955 | 0.072319 |
| CSF1R | −0.40535 | 0.031354 | 0.090928 |
| CSF3R | −0.06577 | 0.602103 | 0.678296 |
| CTLA4 | 0.368167 | 7.57E−05 | 0.004309 |
| CTNNB1 | −0.61501 | 0.0004 | 0.009276 |
| CTSL | −0.3943 | 0.152108 | 0.284503 |
| CTSS | −0.48815 | 0.056744 | 0.146275 |
| CXCL10 | 0.47569 | 0.004906 | 0.033721 |
| CXCL11 | 0.257268 | 0.030527 | 0.090797 |
| CXCL13 | 0.5935 | 0.000128 | 0.004957 |
| CXCL9 | 0.756073 | 0.000537 | 0.010389 |
| CXCR5 | 0.231871 | 0.059151 | 0.147559 |
| CXCR6 | 0.206875 | 0.009584 | 0.049058 |
| CYBB | −0.20751 | 0.324715 | 0.451101 |
| DAB2 | −0.63965 | 0.002548 | 0.023217 |
| DNMT1 | −0.11056 | 0.129912 | 0.253644 |
| DNMT3A | 0.043667 | 0.593059 | 0.677782 |
| DOCK9 | 0.153536 | 0.102196 | 0.215541 |
| DPP4 | 0.259088 | 0.045565 | 0.124367 |
| DUSP2 | 0.295524 | 0.018383 | 0.069916 |
| DUSP4 | 0.041936 | 0.671959 | 0.735351 |
| DUSP5 | 0.344179 | 0.0077 | 0.04828 |
| EIF2AK2 | 0.069752 | 0.531953 | 0.632887 |
| ENG | −0.62626 | 0.000367 | 0.009276 |
| ENTPD1 | −0.01951 | 0.873945 | 0.897147 |
| EOMES | 0.1271 | 0.173616 | 0.305375 |
| ETS2 | −0.03715 | 0.769468 | 0.815144 |
| EWSR1 | −0.22616 | 0.02397 | 0.076178 |
| EZH2 | 0.240089 | 0.051797 | 0.137567 |
| F13A1 | −0.11055 | 0.557979 | 0.649504 |
| FBP1 | −0.11808 | 0.449808 | 0.568775 |
| FBXW7 | 0.340913 | 0.009653 | 0.049058 |
| FCER1G | −0.38525 | 0.113167 | 0.232342 |
| FCER2 | 0.269362 | 0.025 | 0.077801 |
| FCGR2A | −0.19386 | 0.222704 | 0.358801 |
| FCGR2B | 0.072937 | 0.600125 | 0.678296 |
| FCGRT | −0.58051 | 0.006393 | 0.041198 |
| FCRL2 | 0.270226 | 0.012745 | 0.056862 |
| FLT1 | 0.170433 | 0.322126 | 0.4502 |
| FOXP3 | 0.252392 | 5.31E−05 | 0.004309 |
| FPR1 | −0.0995 | 0.456278 | 0.572198 |
| FPR3 | −0.17361 | 0.262546 | 0.395569 |
| GLS | −0.05307 | 0.66028 | 0.729453 |
| GLUL | −0.24498 | 0.118056 | 0.240255 |
| GNAQ | −0.22244 | 0.052181 | 0.137567 |
| GNLY | 0.14597 | 0.265901 | 0.397993 |
| GZMA | 0.176706 | 0.235401 | 0.366254 |
| GZMB | 0.160326 | 0.131245 | 0.25374 |
| GZMK | 0.09366 | 0.405964 | 0.529121 |
| HAVCR2 | −0.17666 | 0.183751 | 0.315779 |
| HCK | −0.05882 | 0.669037 | 0.735351 |
| HDAC4 | −0.0239 | 0.753909 | 0.802325 |
| HDC | 0.161526 | 0.20944 | 0.342184 |
| HIF1A | 0.013789 | 0.921612 | 0.937763 |
| HLA-DMA | −0.42377 | 0.055177 | 0.143831 |
| HLA-DMB | −0.35567 | 0.179733 | 0.313457 |
| HLA-DOA | −0.14408 | 0.321625 | 0.4502 |
| HLA-DOB | 0.497365 | 8.65E−05 | 0.004309 |
| HLA-DPA1 | −0.29498 | 0.181049 | 0.313457 |
| HLA-DPB1 | 0.322182 | 0.001735 | 0.020131 |
| HLA-DQA1/2 | −0.12997 | 0.55175 | 0.649504 |
| HLA-DQB1 | 0.20552 | 0.092207 | 0.203733 |
| HLA-F | −0.04445 | 0.813907 | 0.854418 |
| ICAM2 | 0.294002 | 0.070247 | 0.166298 |
| IDO1 | 0.261794 | 0.022892 | 0.073762 |
| IFNG | 0.183478 | 0.021796 | 0.072319 |
| IFNGR2 | −0.23491 | 0.025151 | 0.077801 |
| IGF2R | −0.07079 | 0.49135 | 0.603139 |
| IL11RA | 0.20145 | 0.033767 | 0.095781 |
| IL15 | 0.24516 | 0.000749 | 0.011591 |
| IL17RA | 0.023122 | 0.803512 | 0.84734 |
| IL18RAP | 0.121474 | 0.234374 | 0.366254 |
| IL22RA2 | 0.399005 | 0.002004 | 0.021138 |
| IL2RB | 0.048847 | 0.685744 | 0.739965 |
| IL3RA | 0.276373 | 0.020706 | 0.072319 |
| IL4R | −0.12123 | 0.194097 | 0.32869 |
| ILF3 | −0.28191 | 0.011576 | 0.054808 |
| IRAK4 | 0.05516 | 0.657666 | 0.729453 |
| IRF2 | 0.13758 | 0.193837 | 0.32869 |
| ITCH | −0.15581 | 0.067706 | 0.163102 |
| ITGA9 | 0.068825 | 0.515099 | 0.619186 |
| ITGAM | −0.08651 | 0.482617 | 0.597265 |
| ITGAX | −0.39587 | 0.07279 | 0.168874 |
| ITGB2 | −0.58719 | 0.031002 | 0.090928 |
| ITGB7 | 0.228546 | 0.030323 | 0.090797 |
| KIT | −0.2384 | 0.11071 | 0.229328 |
| KLRB1 | 0.224918 | 0.08169 | 0.185805 |
| KLRF1 | 0.271937 | 0.019894 | 0.072319 |
| LAG3 | 0.310389 | 0.000334 | 0.009276 |
| LAIR1 | −0.38111 | 0.04639 | 0.125144 |
| LAMP3 | 0.155462 | 0.275544 | 0.40259 |
| LILRB4 | −0.15131 | 0.521488 | 0.623635 |
| MAP3K1 | 0.114533 | 0.172056 | 0.305375 |
| MAP3K5 | 0.047146 | 0.581393 | 0.667738 |
| MAPK8 | 0.123829 | 0.1578 | 0.290413 |
| MAPKAPK2 | −0.15626 | 0.226222 | 0.361956 |
| MARCO | 0.131407 | 0.510299 | 0.616611 |
| MERTK | −0.32749 | 0.099796 | 0.214377 |
| MNAT1 | 0.355727 | 0.002607 | 0.023217 |
| MRC1 | −0.18388 | 0.397483 | 0.523955 |
| MS4A2 | 0.025261 | 0.828855 | 0.858596 |
| MS4A6A | −0.31052 | 0.120111 | 0.240905 |
| MSR1 | −0.33419 | 0.086474 | 0.192904 |
| MYB | 0.161135 | 0.220817 | 0.358249 |
| NCAM1 | 0.08487 | 0.329799 | 0.452504 |
| NCR1 | 0.153461 | 0.142464 | 0.270667 |
| NFAM1 | −0.09018 | 0.48399 | 0.597265 |
| NFATC1 | 0.137037 | 0.162447 | 0.292153 |
| NFKBIA | −0.49826 | 0.000483 | 0.010191 |
| NKG7 | 0.114293 | 0.333527 | 0.452504 |
| NLRP3 | 0.356779 | 0.003323 | 0.026452 |
| NOS2 | 0.199728 | 0.097403 | 0.211192 |
| NOTCH2 | −0.3127 | 0.009778 | 0.049058 |
| NT5E | 0.297784 | 0.003482 | 0.026452 |
| PAX5 | 0.318709 | 0.009938 | 0.049058 |
| PBX3 | −0.12152 | 0.27397 | 0.40259 |
| PDCD1 | 0.294241 | 9.29E−05 | 0.004309 |
| PDGFD | −0.051 | 0.712527 | 0.765307 |
| PDK1 | 0.594028 | 0.013235 | 0.056862 |
| PECAM1 | −0.19983 | 0.275913 | 0.40259 |
| PIK3CB | 0.006084 | 0.958839 | 0.96299 |
| PIK3R1 | −0.32757 | 0.003535 | 0.026452 |
| PPP3CA | −0.0523 | 0.562035 | 0.649504 |
| PRF1 | 0.128054 | 0.307702 | 0.435783 |
| PRKACB | −0.14001 | 0.234709 | 0.366254 |
| PRKCE | 0.076834 | 0.421382 | 0.54255 |
| PSMB10 | −0.01128 | 0.91288 | 0.932988 |
| PSMB9 | 0.120425 | 0.269016 | 0.400075 |
| PTGS2 | 0.144345 | 0.307896 | 0.435783 |
| RAB7A | −0.64944 | 7.29E−05 | 0.004309 |
| RAC2 | 0.069776 | 0.67549 | 0.735746 |
| RAD21 | −0.1034 | 0.400614 | 0.525099 |
| RASGRF1 | 0.509061 | 0.000662 | 0.010966 |
| RASGRF2 | 0.242188 | 0.033853 | 0.095781 |
| RELB | −0.15156 | 0.251184 | 0.385926 |
| REPS1 | 0.050236 | 0.679337 | 0.736478 |
| RFC3 | 0.200974 | 0.062622 | 0.154556 |
| ROCK1 | −0.01803 | 0.828989 | 0.858596 |
| RPTOR | 0.090841 | 0.289181 | 0.416708 |
| RRAS2 | 0.122756 | 0.204493 | 0.336471 |
| RUNX1 | −0.26718 | 0.008968 | 0.049058 |
| S100A8 | 0.573907 | 0.009816 | 0.049058 |
| SDHA | −0.23669 | 0.120556 | 0.240905 |
| SFXN1 | 0.223601 | 0.020299 | 0.072319 |
| SGK1 | −0.38258 | 0.058778 | 0.147559 |
| SH2D1B | 0.358768 | 0.002448 | 0.023217 |
| SIGLEC1 | 0.100114 | 0.495625 | 0.605184 |
| SIRPA | −0.29677 | 0.058176 | 0.147559 |
| SIRPB2 | 0.083841 | 0.562717 | 0.649504 |
| SNCA | 0.16927 | 0.173748 | 0.305375 |
| SOS1 | 0.064908 | 0.436542 | 0.556471 |
| SPP1 | −0.49736 | 0.361513 | 0.487622 |
| SRP54 | 0.088153 | 0.308053 | 0.435783 |
| STAT2 | −0.09218 | 0.507199 | 0.616075 |
| TBX21 | 0.113164 | 0.161113 | 0.292017 |
| TBXAS1 | −0.26005 | 0.1435 | 0.270667 |
| TCF3 | 0.283122 | 0.010669 | 0.051565 |
| TCL1A | 0.303002 | 0.014799 | 0.061311 |
| TGFBR1 | −0.37644 | 0.002702 | 0.023217 |
| TGFBR2 | −0.33777 | 0.008277 | 0.049058 |
| THEM4 | 0.278471 | 0.028679 | 0.087548 |
| TIGIT | 0.281366 | 0.001027 | 0.013903 |
| TLR1 | 0.033078 | 0.721227 | 0.771081 |
| TLR10 | 0.197252 | 0.130102 | 0.253644 |
| TLR2 | −0.1085 | 0.4196 | 0.54255 |
| TNFRSF13B | 0.2558 | 0.037751 | 0.104266 |
| TNFRSF1B | −0.1921 | 0.101019 | 0.215013 |
| TNFRSF4 | 0.279533 | 0.017747 | 0.068624 |
| TNR | 0.129133 | 0.262576 | 0.395569 |
| TREM2 | −0.2427 | 0.158976 | 0.290413 |
| TSHR | 0.388141 | 0.005237 | 0.034713 |
| TYK2 | 0.055139 | 0.556308 | 0.649504 |
| TYMP | −0.1464 | 0.475425 | 0.593003 |
| WNT10B | 0.358413 | 0.001837 | 0.020294 |

### Discussion

Leveraging single-nucleus RNA-seq of frozen archival PDAC, common biological programs were comprehensively identified among untreated and post-CRT malignant cells and cancer-associated fibroblasts. This refined molecular taxonomy of PDAC (FIG. 3D) allowed us to stratify patients from bulk RNA-seq profiles of their tumors into prognostic risk groups defined by malignant cell and fibroblast program scores. Differences in malignant and stromal cell composition and programs following treatment were detected, including an enrichment of basal-like and differentiated states in malignant cells and contractile phenotypes in CAFs. Integrating the snRNA-seq and spatial profiling data from the same tumors, it was demonstrated how these differences associate with distinct immune microniches.

Although the study does not include matched pre- and post-treatment specimens and the treated cohort size is modest and cannot be stratified by treatment regimen, the data refine and clarify the overarching distinction between basal-like and classical-like programs and how they are affected by CRT. Squamous and mesenchymal subclasses were identified within the basal-like subtype in both the untreated and treated contexts, as well as bi-lineage differentiated classical-pancreatic states and an induced basal-like phenotype that arise in the treated setting. Collectively, the analysis suggests that CRT may drive a shift towards basal-like and differentiated classical phenotypes and away from classical progenitor states, which may be due to a combination of direct selection on pre-existing states and induced plasticity (FIGS. 2C and 3d). The basal-like programs are also intimately connected to immune-activating interferon and TNF-NFkB programs in the malignant cells (FIG. 12), and their enrichment in the CRT context may be conducive to a more activated, immunogenic environment. Thus, at least some of the CRT-resistant cell states enriched or induced by CRT may yield a tumor microenvironment that is more susceptible to some immunotherapies.

Our spatially-resolved transcriptomics analysis further supported the hypothesis that basal-like malignant cell programs may facilitate a greater degree of immune infiltration compared with classical-like programs⁸⁹. Moreover, the immune infiltrates associated with basal-like and classical-like malignant cells were distinct, suggesting potential strategies for differentially targeting these phenotypes, with immune checkpoint inhibitors for the former and myeloid-directed therapies such as CD40 agonists and TGF-beta modulators (e.g., losartan) for the latter. Similar phenomena have been observed in other cancer types such as breast, in which the triple-negative subtype has enhanced basal-like features compared to others and is similarly associated with elevated immune cell infiltration that correlates with greater response to immune checkpoint inhibitors^{127,128}.

The snRNA-seq data also helps address the open question of whether the previously identified exocrine-like and ADEX subtypes of PDAC truly exist or if they represent normal tissue contamination^{7,8,10,11,129-131}. The existence of both exocrine- and endocrine-like cancer cells (with inferred CNAs) was confirmed in post-treatment but not untreated tumors. Thus, it is plausible that endoderm-differentiated cancer cell phenotypes are only prevalent enough to be detected under treatment selection pressure, and may alternatively reflect normal cell contamination in treatment-naïve bulk studies.

The presence of relatively resistant neuroendocrine- and exocrine-like cancer cells after neoadjuvant CRT is also clinically important. Neuroendocrine cells in PDAC and precursor lesions have been shown to promote tumorigenesis via neuronal cross-talk, and may thus be partly responsible for the enrichment in Schwann cells associated with treatment (FIG. 2A)^{130,132}. Moreover, primary PDAC cell lines from xenografts featuring the HNF1A-expressing exocrine subtype¹³⁰ are relatively resistant to small molecule tyrosine kinase inhibitors (TKIs), in a manner dependent on an inducible CYP3A5 that oxidizes and inactivates the TKIs. Notably, CYP3A5 is a member of some of the classical-like topics in the cohort, but CYP3A5 and its regulator HNF4A were repressed in both the post-CRT cohort and its high-residual post-CRT subgroup (FIG. 2C; FIG. 9D; Table 2.1-2.6). NFII2, which modulates drug-induced CYP3A5 upregulation is also repressed in the high-residual post-CRT subset. Taken together, these results suggest that CRT may sensitize residual basal-like cancer cells to TKIs and warrants further investigation.

Overall, this study provides a high-resolution molecular framework for understanding the intra-tumoral diversity of pancreatic cancer and treatment-associated changes, spatial associations among malignant/fibroblast-intrinsic programs and both quantitative and qualitative differences in immune microniches, and clinically-relevant prognostication. These findings can be harnessed to augment precision oncology efforts in pancreatic cancer.

### Methods

### Human Patient Specimens

For inclusion in this study, patients had non-metastatic pancreatic ductal adenocarcinoma and went to surgical resection with or without neoadjuvant radiotherapy and/or chemotherapy. Some patients received additional neoadjuvant therapy in the form of immune checkpoint inhibitors or losartan, an angiotensin II receptor type 1 antagonist. All patients were consented to protocol 2003P001289 (principal investigator: CFC; co-investigators: ASL, WLH), which was reviewed and approved by the Massachusetts General Hospital (MGH) Institutional Review Board. Resected primary tumor samples were examined to confirm neoplastic content by a board-certified pathologist (MMK) and then snap frozen and stored at −80° C. for up to 7 years prior to processing. Specimens were screened for an RNA integrity number (RIN; Agilent RNA 6000 Pico Kit, cat. No. 5067-1513) greater than an empirically determined threshold of 6; only specimens with RIN>6 were processed further.

Nucleus Isolation from Frozen Samples

The recently published toolbox for snRNA-seq of tumors spanning a broad range of nucleus isolation techniques for various tissue/tumor types⁴³ does not include PDAC. The following protocol is an adaptation and optimization of this prior work specifically for the unique tissue requirements of pancreatic tumors. A 2× stock of STc buffer in nuclease-free water was prepared with a final concentration of 292 mM NaCl (ThermoFisher Scientific, cat. no. AM9759), 40 mM Tricine (VWR, cat. no. E170-100G), 2 mM CaCl₂ (VWR, cat. no. 97062-820), and 42 mM MgCl₂ (Sigma Aldrich, cat. no. M1028). For each specimen, 2 mL of NSTcPA buffer was prepared by combining 1 mL of 2× STc buffer, 40 μL of 10% Nonidet P40 Substitute (Fisher Scientific, cat. no. AAJ19628AP), 10 μL of 2% bovine serum albumin (New England Biolabs, cat. no. B9000S), 0.3 μL of 1M spermine (Sigma-Aldrich, cat. no. S3256-1G), 1 μL of 1M spermidine (Sigma-Aldrich, cat. no. S2626-1G), and 948.7 μL of nuclease-free water. For each specimen, 3 mL of 1x working STc buffer was made by diluting 2× STc 1:1 in nuclease-free water.

NSTcPA buffer (1 mL) was pipetted into one well of a 6-well plate (Stem Cell Technologies, cat. no. 38015) on ice. The frozen tumor specimen was removed from −80° C. and placed in a petri dish on dry ice. Using a clean razor blade, the desired regions of the tissue were cut while the specimen remained frozen (at least 10-20 mg). The remainder of the specimen was returned to −80° C. for subsequent use. The selected tissue was transferred into the NSTcPA buffer and manually minced with fine straight tungsten carbide scissors (Fine Science Tools, cat. no. 14568-12) for 8 minutes. The homogenized tissue solution was then filtered through a 40 μm Falcon cell filter (Thermo Fisher Scientific, cat. no. 08-771-1) into a 50 mL conical tube. An additional 1 mL of NSTcPA buffer was used to rinse the well and filter. The total volume was brought up to 5 mL with 3 mL of 1× STc buffer and transferred into a 15 mL conical tube. The sample was spun for 5 min at 500 xg, 4° C. and the supernatant was removed. The pellet was resuspended in 100-200 μL 1× STc and then filtered through a 35 μm Falcon cell strainer (Corning, cat. no. 352235). Nuclei were quantified using a C-chip disposable hemocytometer (VWR, cat. no. 82030-468) and diluted in 1× STc as necessary to achieve a final concentration of 300-2,000 nuclei/μL.

SINGLE-Nucleus RNA-Seq (snRNA-Seq)

Approximately 8,000 nuclei per sample were loaded into each channel of a Chromium single-cell 3′ chip (V2 or V3, 10× Genomics) according to the manufacturer's instructions. Single nuclei were partitioned into droplets with gel beads in the Chromium Controller to form emulsions, after which nucleus lysis, barcoded reverse transcription of mRNA, cDNA amplification, enzymatic fragmentation, and 5′ adaptor and sample index attachment were performed according to manufacturer's instructions. Up to four sample libraries were sequenced on the HiSeq X Version 2.5 (Illumina) with the following paired end read configuration: read 1, 26-28 nt; read 2, 96-98 nt; index read, 8 nt.

snRNA-Seq Data Pre-Processing

BCL files were converted to FASTQ using bcl2fastq2-v2.20. CellRanger v3.0.2 was used to demultiplex the FASTQ reads, align them to the hg38 human transcriptome (pre-mRNA) and extract the UMI and nuclei barcodes. The output of this pipeline is a digital gene expression (DGE) matrix for each sample, which has quantified for each nucleus barcode the number of UMIs that aligned to each gene.

Low-quality nuclei profiles were filtered by baseline quality control measures. First, profiles were discarded with fewer than 400 genes expressed or with greater than 20% of reads originating from mitochondrial genes. Additionally, doublet detection was performed over all nuclei profiles by using Scrublet¹³³ and removed all profiles with a Scrublet score greater than 0.2. To account for differences in sequencing depth across nuclei, UMI counts were normalized by the total number of UMIs per nucleus and converted to transcripts-per-10,000 (TP10K) as the final expression unit.

### Dimensionality Reduction, Clustering and Annotation

Following these quality control steps, treatment-naïve and neoadjuvant-treated specimens were aggregated into two separate datasets. The log₂(TP10K+1) expression matrix for each dataset was used for the following downstream analyses. For each dataset, the top 2,000 highly variable genes across the entire dataset were identified using the Scanpy¹³⁴ highly variable genes function with the sample id as input for the batch, and then performed a Principal Component Analysis (PCA) over the top 2,000 highly variable genes and identified the top 40 principle components (PCs) beyond which negligible additional variance was explained in the data (the analysis was performed with 30, 40, and 50 PCs and robust to this choice). Subsequently, a k-nearest neighbors graph of nuclei profiles (k=10) was built based on the top 40 PCs and performed community detection on this neighborhood graph using the Leiden graph clustering method¹³⁵. Distinct cell populations were identified and annotated using known cell type-specific gene expression signatures^{40,47-49}. Individual nuclei profiles were visualized using the uniform manifold approximation and projection (UMAP)⁴⁴.

Inferring Copy Number Aberrations from Single Nucleus Profiles

InferCNV v3.9¹³⁶ was run on all nuclei profiles for each tumor separately with a common set of high confidence non neoplastic cells used as the reference. A 100 gene window was used in sub-clustering mode and an HMM to predict the copy number aberration (CNA) count in each nucleus.

### Multiplexed Ion Beam Imaging (MIBI)

Formalin-fixed paraffin-embedded pancreatic tissue sections were cut onto gold MIBI slides (IONpath, cat. no. 567001) and stained at IONpath (Menlo Park, CA) with the internal Epithelial i-Onc isotopically-labelled antibody panel (IONpath): dsDNA_89 [3519 DNA] (1:100), β-tubulin_166 [D3U1W] (3:200), CD163_142 [EPR14643-36] (3:1600), CD4_143 [EPR6855] (1:100), CD11c_144 [EP1347Y] (1:100), LAG3_147 [17B4] (1:250), PD-1_148 [D4W2J] (1:100), PD-L1_149 [E1L3N] (1:100), Granzyme B_150 [D6E9W](1:400), CD56_151 [MRQ-42] (1:1000), CD31_152 [EP3095] (1:1000), K1-67_153 [D2H10](1:250), CD11b_155 [D6X₁N] (1:500), CD68_156 [D4B9C] (1:100), CD8_158 [C8/144B](1:100), CD3_159 [D7A6E] (1:100), CD45RO_161 [UCHL1] (1:100), Vimentin_163 [D21H3] (1:100), Keratin_165 [AE1/AE3] (1:100), CD20_167 [L26] (1:400), Podoplanin_170 [D2-40] (1:100), IDO1_171 [EPR20374] (1:100), HLA-DR_172 [EPR3692] (1:100), DC-SIGN_173 [DCN46] (1:250), CD45_175 [2B11 & PD7/26] (3:200), HLA class 1 A, B, and C_176 [EMR8⁻⁵] (1:100), Na/K-ATPase_176 [D4Y7E] (1:100).

Quantitative imaging was performed using a beta unit MIBIscope (IONpath) equipped with a duoplasmatron ion source. This instrument sputters samples with O₂⁺ primary ions line-by-line, while detecting secondary ions with a time-of-flight mass spectrometer tuned to 1-200 m/z+ and mass resolution of 1000 m/Δm, operating at a 100 KHz repetition rate. The primary ion beam was aligned daily to minimize imaging astigmatism and ensure consistent secondary ion detection levels using a built-in molybdenum calibration sample. In addition to the secondary ion detector, the MIBIscope is equipped with a secondary electron detector which enables sample identification and navigation prior to imaging.

For data collection, three fields of view were acquired for each sample by matching the secondary electron morphological signal to annotated locations on sequential H&E stained slides. The experimental parameters used in acquiring all imaging runs were as follows: pixel dwell time (12 ms), image size (500 μm² at 1024×1024 pixels), primary ion current (5 nA O₂⁺), aperture (300 m), stage bias (+67 V).

### MIBI Image Processing Segmentation and Quantification

Mass spectrometer run files were converted to multichannel tiff images using MIB.io software (IONpath). Mass channels were filtered individually to remove gold-ion background and spatially uncorrelated noise. HLA Class 1 and Na/K-ATPase signals were combined into a single membrane marker. These image files (tiff) were used as a starting point for single cell segmentation, quantification and interactive analysis using histoCAT (v1.76)¹³⁷. A similar approach was followed for segmentation as proposed for Imaging Mass Cytometry data¹³⁷⁻¹³⁹. Briefly, Ilastik¹⁴⁰ was used to manually train three classes (nuclei, cytoplasm and background) to improve subsequent watershed segmentation using CellProfiler¹⁴¹. Finally, the tiff images and masks were combined for histoCAT loading with a script optimized for MIBI image processing. All code, classifiers and configuration files are available at github.com/DenisSch/MIBI

### Differential Gene Expression Analysis

For each annotated cell type detected in both untreated and treated tumors, a differential gene analysis was performed between cells in the two populations to identify upregulated and downregulated genes. A Wilcoxon statistical test was used to compute the p-values for each gene and Bonferroni correction was applied to correct for multiple testing.

### Scoring Gene Signatures for Each Nucleus Profile

A signature score for each nucleus profile was computed as the mean log₂(TP10K+1) expression across all genes in the gene signature. Subsequently, to identify statistically significant gene expression patterns, The mean log₂(TP10K+1) expression was computed across a background set of 50 genes randomly selected with matching expression levels to those of the genes in the signature iterated 25 times. The gene signature score was defined to be the excess in expression found across the genes in the gene signature compared to the background set.

### Cell-Cell Interaction Analysis by Receptor-Ligand Pair Expression

To characterize potential cell-cell interactions, identifying pairs of cell types where one expresses a receptor gene and the other expresses its cognate ligand was attempted. First, known receptor ligand pairs defined in the FANTOM5 receptor ligand database¹⁴² were identified. Next, a log fold change and p-value was computed for the expression of each gene in each cell type vs. profiles from all other cell types to identify genes differentially expressed in each cell type (separately for untreated and treated data).Receptor ligand pairs where either receptor or ligand have a log fold change below 1.5 were discarded, and a Receptor-Ligand (RL) score was calculated by multiplying the log₂ fold change of the receptor (in cell type i) and ligand (in cell type j) to give higher priority to receptor ligand pairs that were highly specific to the respective cell types.

### Consensus Non-Negative Matrix Factorization

The task of dissecting gene expression programs was formulated as a matrix factorization problem where the input gene expression matrix is decomposed into two matrices =x s. t. W, H >0. The solution to this formulation can be identified by solving the following minimization problem:

argmin{+(1−α)∥∥

+(1−α)++}

The non-negative matrix factorization implemented in sklearn was utilized to derive the tumor and CAF expression programs. Because the result of NMF optimization can vary between runs based on random seeding, and repeated NMF 50 times per cell type category and computed a set of consensus programs by aggregating results from all 50 runs and computed a stability and reconstruction error. This consensus NMF was performed by making custom updates to the cNMF python package. To determine the optimal number of programs (p) for each cell type and condition, balanced between maximizing stability and minimizing error of the cNMF solution, while ensuring that the resulting programs were as biologically coherent and parsimonious as possible. Each program was annotated utilizing a combination of GSEA¹⁴³ and comparison to bulk expression signatures.

### Survival Analysis of Bulk RNA-Seq Data

Bulk RNA-seq data from two previously published resected primary PDAC cohorts with overall survival annotated were obtained (The Cancer Genome Atlas, n=139; PanCuRx, n=168)^{11,78}. Patients with metastases or those that received neoadjuvant therapy were excluded from this analysis. Gene expression levels from RNA-seq data was estimated using RSEM¹⁴⁴.

To score untreated malignant and fibroblast programs in each tumor, calculated cNMF-weighted expression scores for each program and normalized the expression scores by calculating z-scores. Notably, for malignant programs the five lineage programs were scored (due to the overlap between cell state and lineage programs and to reduce complexity). For fibroblasts, all four programs were scored.

For the malignant only analysis, each tumor for the five untreated malignant lineage cNMF programs (normalized by z-scores) were scored, and identified the top scoring program (primary program) as well as the number of highly-expressed programs defined as expression greater than the mean of the cohort (heterogeneity score, H). Patients with 0 or 1 highly-expressed programs were assigned H=0, those with 2 highly-expressed programs were assigned H=1, and those with 3 or more highly-expressed programs were assigned H=2, and then stratified each tumor into one of 15 groups based on the combination of the primary program (5) and heterogeneity score (3) (FIG. 3E, row labels). Analogously, for the fibroblast only analysis, each tumor was scored based on primary program (4) and heterogeneity score (2) to stratify each tumor into one of 8 groups (FIG. 3E, column labels). For the fibroblasts, there was one fewer program than the malignant cells so patients with 0 or 1 high-expressed programs were scored with H=0 and those with 2 or more high-expressed programs with H=1.

Next, the tumors were partitioned in the malignant- and fibroblast-only analyses into three risk groups: low, intermediate, high. To this end, preliminary Kaplan-Meier (KM) analyses were performed for overall survival (OS) based on each individual primary program, heterogeneity score, and both (FIG. 13A-13F). The survival curves were inspected to consider any prognosis trends and used this information to assign combinations of primary programs and heterogeneity score values into low, intermediate and high-risk groups pre-hoc (i.e., prior to the KM analyses of FIG. 3F-3G). This was done separately for the malignant cells (FIG. 3E, left vertical red color bar) and the fibroblasts (FIG. 3E, top horizontal blue color bar).

To assign tumors into risk strata based on both malignant and fibroblast program scores and heterogeneity indices, the tumor assignment to malignant and fibroblast risk groups above were used, which defined nine combinations (matrix entries in FIG. 3E, FIG. 13G), and then grouped possible combinations into four risk strata: both low (“low”), both high (“high”), one high and one intermediate (“high-intermediate”), and all other combinations (“low-intermediate”) (color grid inside matrix in FIG. 3E, purple color legend).

Finally, survival analyses were performed for the risk stratified data based on three malignant risk groups (FIG. 3F), three fibroblast risk groups (FIG. 3G), and four combined risk groups (FIG. 311). Survival analysis was performed using the Kaplan-Meier estimate and the survival distributions for each patient strata were compared using the log-rank test for equality of survivor functions (Stata/SE 15.1).

### Digital Spatial Profiling (DSP)

Published experimental methods¹²⁵ were used with modifications as noted below. Briefly, formalin-fixed paraffin-embedded (FFPE) sections (5 μm) of 12 specimens (8 untreated, 4 treated) were prepared by the MGH Histopathology Core on the IRB-approved protocol (2003P001289). Slides were baked at 37° C. overnight, deparaffinized, rehydrated, antigen-retrieved in pressure cooker for 20 min at 100° C. and low pressure, proteinase-K digestion for 15 min, post-fixed in neutral-buffered formalin for 10 min, hybridized to UV-photocleavable barcode-conjugated RNA in situ hybridization probe set (cancer transcriptome atlas/CTA with 1,412 targets or whole transcriptome atlas/WTA) overnight, washed to remove off-target probes, and then counterstained with morphology markers for 2 hours. The morphology markers consisted of: 1:10 SYTO13 (ThermoFisher Scientific, cat. no. 57575), 1:40 anti-panCK-Alexa Fluor 532 (clone AE-1/AE-3; Novus Biologicals, cat. no. NBP2-33200AF532), 1:40 anti-CD45-Alexa Fluor 594 (clone 2B11+PD7/26; Novus Biologicals, cat. no. NBP2-34528AF594), and 1:100 anti-aSMA-Alexa Fluor 647 (clone 1A4; Novus Biologicals, cat. no. IC1420R). These four morphology markers allowed delineation of the nuclear, epithelial, immune, and fibroblast compartments, respectively. Immunofluorescence images, region of interest (ROI) selection, segmentation into marker-specific areas of interest (AOI), and spatially-indexed barcode cleavage and collection were performed on a GeoMx Digital Spatial Profiling instrument (NanoString) using either the pre-commercial Cancer Transcriptome Atlas (CTA) probe set or a whole transcriptome atlas assay (NanoString). Approximately 8-10 ROIs and 23-25 AOIs were collected per specimen. Library preparation was performed according to the manufacturer's instructions and involved PCR amplification to add Illumina adapter sequences and unique dual sample indices. Up to 96 AOIs were pooled and sequenced on a NextSeq High Output v2.5 (75 cycles, 2×38 bp; Illumina, cat. no. 20024906).

### Computational Analysis of DSP Data

FASTQ files for DSP were aggregated into count matrices as described previously¹²⁵. Briefly, deduplicated sequencing counts were calculated based on UMI and molecular target tag sequences. Outlier probes were removed per target when multiple probes were available, and target expression values were calculated as the geometric mean of the remaining probes. Single probe genes were reported as the deduplicated count value. The limit of quantitation (LOQ) was estimated as the geometric mean of the negative control probes plus 2 geometric standard deviations of the negative control probes. Targets were removed that consistently fell below the LOQ, and the datasets were normalized using upper quartile (Q3) normalization.

Statistical analysis was performed using R. For DSP analysis of individual data points, when feasible, linear mixed effect models¹⁴⁵ were used to control for multiple sampling within a slide, using Satterthwaite's approximation¹⁴⁶ for degrees of freedom for p-value calculation. When replication was insufficiently powered for mixed effect models, Student's t-test was used to test associations with subtype classification. All analyses were two-sided and used a significant level of p-value ≤0.05 and were adjusted for multiple testing where appropriate using the false discovery rate¹⁴⁷. Programs were scored for each DSP sample within each region of interest using single-sample gene set enrichment analysis (ssGSEA)¹⁴⁸. To further alignthe malignant programs with the conventional classification, comparisons between classical-like and basal-like subtypes were performed after mean-centering the various cNMF program scores within these aggregate categories as described previously.

### Data Availability

Raw data will be available in the controlled access repository Data Use Oversight System (DUOS) at the Broad Institute: duos.broadinstitute.org/under its Data Access Committee. Processed annotated datasets will be provided in GEO and the Single Cell Portal upon publication.

### Code Availability

All code will be available upon publication in Github at github.com/karthikj89/humanpdac.

### REFERENCES

1. Rahib, L. et al. Projecting cancer incidence and deaths to 2030: The unexpected burden of thyroid, liver, and pancreas cancers in the united states. Cancer Research vol. 74 2913-2921 (2014).
2. Siegel, R. L., Miller, K. D. & Jemal, A. Cancer statistics, 2020. CA. Cancer J. Clin. 70, 7-30 (2020).
3. Ben-Aharon, I. et al. Genomic landscape of pancreatic adenocarcinoma in younger versus older patients: Does age matter? Clin. Cancer Res. 25, 2185-2193 (2019).
4. Siegel, R. et al. Cancer treatment and survivorship statistics, 2012. CA. Cancer J. C/in. 62, 220-241 (2012).
5. Versteijne, E. et al. Preoperative Chemoradiotherapy Versus Immediate Surgery for Resectable and Borderline Resectable Pancreatic Cancer: Results of the Dutch Randomized Phase III PREOPANC Trial. J. Clin. Oncol. 38, 1763-1773 (2020).
6. Murphy, J. E. et al. Total Neoadjuvant Therapy with FOLFIRINOX in Combination with Losartan Followed by Chemoradiotherapy for Locally Advanced Pancreatic Cancer: A Phase 2 Clinical Trial. JAMA Oncol. 5, 1020-1027 (2019).
7. Collisson, E. A., Bailey, P., Chang, D. K. & Biankin, A. V. Molecular subtypes of pancreatic cancer. Nature Reviews Gastroenterology and Hepatology vol. 16 207-220 (2019).
8. Collisson, E. A. et al. Subtypes of pancreatic ductal adenocarcinoma and their differing responses to therapy. Nat. Med. 17, 500⁻⁵⁰³ (2011).
9. Moffitt, R. A. et al. Virtual microdissection identifies distinct tumor- and stroma-specific subtypes of pancreatic ductal adenocarcinoma. Nat. Genet. 47, 1168-1178 (2015).
10. Bailey, P. et al. Genomic analyses identify molecular subtypes of pancreatic cancer. Nature 531, 47(2016).
11. Raphael, B. J. et al. Integrated Genomic Characterization of Pancreatic Ductal Adenocarcinoma. Cancer Cell 32, 185-203.e13 (2017).
12. Sivakumar, S., de Santiago, I., Chlon, L. & Markowetz, F. Master Regulators of Oncogenic KRAS Response in Pancreatic Cancer: An Integrative Network Biology Analysis. PLoS Med. 14, (2017).
13. de Santiago, I. et al. Immuno-phenotypes of Pancreatic Ductal Adenocarcinoma: Metaanalysis of transcriptional subtypes. bioRxiv (2017) doi:10.1101/198903.
14. Aung, K. L. et al. Genomics-driven precision medicine for advanced pancreatic cancer: Early results from the COMPASS trial. Clin. Cancer Res. (2018) doi:10.1158/1078-0432.CCR-17-2994.
15. Kroemer, G., Galluzzi, L., Kepp, O. & Zitvogel, L. Immunogenic cell death in cancer therapy. Annual Review of Immunology vol. 31 51-72 (2013).
16. Coffelt, S. B. & de Visser, K. E. Immune-mediated mechanisms influencing the efficacy of anticancer therapies. Trends in Immunology vol. 36 198-216 (2015).
17. Zitvogel, L., Apetoh, L., Ghiringhelli, F. & Kroemer, G. Immunological aspects of cancer chemotherapy. Nature Reviews Immunology vol. 8 59-73 (2008).
18. Burnette, B. C. et al. The efficacy of radiotherapy relies upon induction of type I interferon-dependent innate and adaptive immunity. Cancer Res. 71, 2488-2496 (2011).
19. Gupta, A. et al. Radiotherapy Promotes Tumor-Specific Effector CD8⁺ T Cells via Dendritic Cell Activation. J. Immunol. 189, 558-566 (2012).
20. Ma, Y. et al. Anticancer chemotherapy-induced intratumoral recruitment and differentiation of antigen-presenting cells. Immunity 38, 729-741 (2013).
21. Zitvogel, L., Galluzzi, L., Kepp, O., Smyth, M. J. & Kroemer, G. Type I interferons in anticancer immunity. Nature Reviews Immunology vol. 15 405-414 (2015).
22. Deng, L. et al. STING-dependent cytosolic DNA sensing promotes radiation-induced type I interferon-dependent antitumor immunity in immunogenic tumors. Immunity 41, 843-852 (2014).
23. Minn, A. J. Interferons and the Immunogenic Effects of Cancer Therapy. Trends in Immunology vol. 36 725-737 (2015).
24. Benci, J. L. et al. Opposing Functions of Interferon Coordinate Adaptive and Innate Immune Responses to Cancer Immune Checkpoint Blockade. Cell 178, 933-948.e14 (2019).
25. Kalluri, R. The biology and function of fibroblasts in cancer. Nature Reviews Cancer vol. 16 582⁻⁵⁹⁸ (2016).
26. Sun, Q. et al. The impact of cancer-associated fibroblasts on major hallmarks of pancreatic cancer. Theranostics vol. 8 5072⁻⁵⁰⁸⁷ (2018).
27. Liu, T. et al. Cancer-associated fibroblasts: An emerging target of anti-cancer immunotherapy. Journal of Hematology and Oncology vol. 12 (2019).
28. Özdemir, B. C. et al. Depletion of carcinoma-associated fibroblasts and fibrosis induces immunosuppression and accelerates pancreas cancer with reduced survival. Cancer Cell 25, 719-734 (2014).
29. Patel, A. P. et al. Single-cell RNA-seq highlights intratumoral heterogeneity in primary glioblastoma. Science (80-.). 344, 1396-1401 (2014).
30. Tirosh, I. et al. Single-cell RNA-seq supports a developmental hierarchy in human oligodendroglioma. Nature 539, 309-313 (2016).
31. Venteicher, A. S. et al. Decoupling genetics, lineages, and microenvironment in IDH-mutant gliomas by single-cell RNA-seq. Science (80-.). 355, (2017).
32. Puram, S. V et al. Single-Cell Transcriptomic Analysis of Primary and Metastatic Tumor Ecosystems in Head and Neck Cancer. Cell 171, 1611-1624.e24 (2017).
33. Azevedo-Pouly, A. C. P., Elgamal, O. A. & Schmittgen, T. D. RNA isolation from mouse pancreas: A ribonuclease-rich tissue. J. Vis. Exp. (2014) doi:10.3791/51779.
34. Augereau, C., Lemaigre, F. P. & Jacquemin, P. Extraction of high-quality RNA from pancreatic tissues for gene expression studies. Anal. Biochem. 500, 60-62 (2016).
35. Jun, E. et al. Method Optimization for Extracting High-Quality RNA From the Human Pancreas Tissue. Transl. Oncol. 11, 800-807 (2018).
36. Von Ahrens, D., Bhagat, T. D., Nagrath, D., Maitra, A. & Verma, A. The role of stromal cancer-associated fibroblasts in pancreatic cancer. Journal of Hematology and Oncology vol. 10 (2017).
37. Habib, N. et al. Div-Seq: Single-nucleus RNA-Seq reveals dynamics of rare adult newborn neurons. Science (80-.). 353, 925-928 (2016).
38. Habib, N. et al. Massively parallel single-nucleus RNA-seq with DroNc-seq. Nat. Methods 14, 955-958 (2017).
39. Drokhlyansky, E. et al. The enteric nervous system of the human and mouse colon at a single-cell resolution. bioRxiv (2019) doi:10.1101/746743.
40. Tosti, L. et al. Single nucleus and in situ RNA sequencing reveals cell topographies in the human pancreas. bioRxiv (2020).
41. Barthelson, R. A., Lambert, G. M., Vanier, C., Lynch, R. M. & Galbraith, D. W. Comparison of the contributions of the nuclear and cytoplasmic compartments to global gene expression in human cells. BMC Genomics (2007) doi:10.1186/1471-2164-8-340.
42. Abdelmoez, M. N. et al. SINC-seq: Correlation of transient gene expressions between nucleus and cytoplasm reflects single-cell physiology. Genome Biol. 19, (2018).
43. Slyper, M. et al. A single-cell and single-nucleus RNA-Seq toolbox for fresh and frozen human tumors. Nat. Med. 26, 792-802 (2020).
44. McInnes, L., Healy, J. & Melville, J. UMAP: Uniform Manifold Approximation and Projection for Dimension Reduction. (2018).
45. Tirosh, I. et al. Dissecting the multicellular ecosystem of metastatic melanoma by single-cell RNA-seq. Science (80-.). (2016) doi:10.1126/science.aad0501.
46. Izar, B. et al. A single-cell landscape of high-grade serous ovarian cancer. Nat. Med. (2020) doi:10.1038/s41591-020-0926-0.
47. Gentles, A. J. et al. The prognostic landscape of genes and infiltrating immune cells across human cancers. Nat. Med. 21, 938-945 (2015).
48. Muraro, M. J. et al. A Single-Cell Transcriptome Atlas of the Human Pancreas. Cell Syst. 3, 385-394.e3 (2016).
49. Zilionis, R. et al. Single-Cell Transcriptomics of Human and Mouse Lung Cancers Reveals Conserved Myeloid Populations across Individuals and Species. Immunity 50, 1317-1334.e10 (2019).
50. Bernard, V. et al. Single Cell Transcriptomics of Pancreatic Cancer Precursors Demonstrates Epithelial and Microenvironmental Heterogeneity as an Early Event in Neoplastic Progression. Clin. Cancer Res. (2018) doi:10.1158/1078-0432.CCR-18-1955.
51. Elyada, E. et al. Cross-species single-cell analysis of pancreatic ductal adenocarcinoma reveals antigen-presenting cancer-associated fibroblasts. Cancer Discovery vol. 9 1102-1123 (2019).
52. Moncada, R. et al. Integrating microarray-based spatial transcriptomics and single-cell RNA-seq reveals tissue architecture in pancreatic ductal adenocarcinomas. Nat. Biotechnol. 38, 333-342 (2020).
53. Angelo, M. et al. Multiplexed ion beam imaging of human breast tumors. Nat. Med. 20, 436-442 (2014).
54. Keren, L. et al. A Structured Tumor-Immune Microenvironment in Triple Negative Breast Cancer Revealed by Multiplexed Ion Beam Imaging. Cell 174, 1373-1387.e19 (2018).
55. Ahmadu-Suka, F. et al. Pathologic response of the pancreas and duodenum to experimental intraoperative irradiation. Int. J Radiat. Oncol. Biol. Phys. 14, 1197-1204 (1988).
56. Yen, T. W. F. et. al. Myofibroblasts are responsible for the desmoplastic reaction surrounding human pancreatic carcinomas. Surgery 131, 129-134 (2002).
57. Bakst, R. L. et al. Are Schwann Cells a Target in Radiation for Perineural Invasion? Int. J. Radiat. Oncol. 87, S629-S630 (2013).
58. Li, Q. et al. Reg proteins promote acinar-to-ductal metaplasia and act as novel diagnostic and prognostic markers in pancreatic ductal adenocarcinoma. Oncotarget 7, 77838-77853 (2016).
59. Liu, X. et al. REG3A accelerates pancreatic cancer cell growth under IL-6-associated inflammatory condition: Involvement of a REG3A-JAK2/STAT3 positive feedback loop. Cancer Lett. 362, 45-60 (2015).
60. Maier, B. et al. A conserved dendritic-cell regulatory program limits antitumour immunity. Nature 580, 257-262 (2020).
61. Gao, Y. et al. Control of T helper 2 responses by transcription factor IRF4-dependent dendritic cells. Immunity 39, 722-732 (2013).
62. Koucký, V., Bou\ddot{c}ek, J. & Fialová, A. Immunology of plasmacytoid dendritic cells in solid tumors: A brief review. Cancers vol. 11 (2019).
63. Guilliams, M. et al. Unsupervised High-Dimensional Analysis Aligns Dendritic Cells across Tissues and Species. Immunity 45, 669-684 (2016).
64. O'Hara, M. H. et al. Abstract CT004: A Phase Ib study of CD40 agonistic monoclonal antibody APX005M together with gemcitabine (Gem) and nab-paclitaxel (NP) with or without nivolumab (Nivo) in untreated metastatic ductal pancreatic adenocarcinoma (PDAC) patients. CT004-CT004 (2019) doi:10.1158/1538-7445.sabcs18-ct004.
65. Blake, S. J., Dougall, W. C., Miles, J. J., Teng, M. W. L. & Smyth, M. J. Molecular pathways: Targeting CD96 and TIGIT for cancer immunotherapy. Clin. Cancer Res. 22, 5183(2016).
66. Tarique, A. A. et al. Phenotypic, functional, and plasticity features of classical and alternatively activated human macrophages. Am. J. Respir. Cell Mol. Biol. 53, 676-688 (2015).
67. Ostuni, R., Kratochvill, F., Murray, P. J. & Natoli, G. Macrophages and cancer: From mechanisms to therapeutic implications. Trends in Immunology vol. 36 229-239 (2015).
68. Murray, P. J. et al. Macrophage Activation and Polarization: Nomenclature and Experimental Guidelines. Immunity vol. 41 14-20 (2014).
69. Calderon, B. et al. The pancreas anatomy conditions the origin and properties of resident macrophages. J. Exp. Med. 212, 1497-1512 (2015).
70. Murphy, J. E. et al. Total neoadjuvant therapy with FOLFIRINOX followed by individualized chemoradiotherapy for borderline resectable pancreatic adenocarcinoma: A phase 2 clinical trial. JAMA Oncol. 4, 963-969 (2018).
71. Weichselbaum, R. R. et al. An interferon-related gene signature for DNA damage resistance is a predictive marker for chemotherapy and radiation for breast cancer. Proc. Natl. Acad. Sci. U.S.A 105, 18490-18495 (2008).
72. Sistigu, A. et al. Cancer cell-autonomous contribution of type I interferon signaling to the efficacy of chemotherapy. Nat. Med. 20, 1301-1309 (2014).
73. Mills, B. N. et al. Stereotactic Body Radiation and Interleukin-12 Combination Therapy Eradicates Pancreatic Tumors by Repolarizing the Immune Microenvironment. Cell Rep. 29, 406-421.e5 (2019).
74. Halenius, A., Gerke, C. & Hengel, H. Classical and non-classical MHC I molecule manipulation by human cytomegalovirus: So many targets—But how many arrows in the quiver? Cellular and Molecular Immunology vol. 12 139-153 (2015).
75. Schmidt, J. et al. Open-label, multicenter, randomized phase III trial of adjuvant chemoradiation plus interferon alfa-2b versus fluorouracil and folinic acid for patients with resected pancreatic adenocarcinoma. J. Clin. Oncol. 30, 4077-4083 (2012).
76. Booy, S., Hofland, L. & Van Eijck, C. Potentials of interferon therapy in the treatment of pancreatic cancer. J. Interf Cytokine Res. 35, 327-339 (2015).
77. Reits, E. A. et al. Radiation modulates the peptide repertoire, enhances MHC class I expression, and induces successful antitumor immunotherapy. J. Exp. Med. 203, 1259-1271 (2006).
78. Chan-Seng-Yue, M. et al. Transcription phenotypes of pancreatic cancer are driven by genomic events during tumor evolution. Nat. Genet. 52, 231-240 (2020).
79. Somerville, T. D. D. et al. TP63-Mediated Enhancer Reprogramming Drives the Squamous Subtype of Pancreatic Ductal Adenocarcinoma. Cell Rep. 25, 1741-1755.e7 (2018).
80. Decker, K., Goldman, D. C., L. Grasch, C. & Sussel, L. Gata6 is an important regulator of mouse pancreas development. Dev. Biol. 298, 415-429 (2006).
81. Porter, R. L. et al. Epithelial to mesenchymal plasticity and differential response to therapies in pancreatic ductal adenocarcinoma. Proc. Natl. Acad. Sci. U.S.A 116, 26835-26845 (2019).
82. Martinez, M. & Moon, E. K. CAR T cells for solid tumors: New strategies for finding, infiltrating, and surviving in the tumor microenvironment. Front. Immunol. 10, 128 (2019).
83. Seino, T. et al. Human Pancreatic Tumor Organoids Reveal Loss of Stem Cell Niche Factor Dependence during Disease Progression. Cell Stem Cell 22, 454-467.e6 (2018).
84. Arensman, M. D. et al. WNT7B mediates autocrine Wnt/β-catenin signaling and anchorage-independent growth in pancreatic adenocarcinoma. Oncogene 33, 899-908 (2014).
85. Juiz, N. et al. Basal-like and classical cells coexist in pancreatic cancer revealed by single-cell analysis on biopsy-derived pancreatic cancer organoids from the classical subtype. FASEB J. (2020) doi:10.1096/fj.202000363RR.
86. Bielecki, P. et al. Skin inflammation driven by differentiation of quiescent tissue-resident ILCs into a spectrum of pathogenic effectors. bioRxiv.
87. Beuran, M. et al. The epithelial to mesenchymal transition in pancreatic cancer: A systematic review. Pancreatology vol. 15 217-225 (2015).
88. Aiello, N. M. et al. EMT Subtype Influences Epithelial Plasticity and Mode of Cell Migration. Dev. Cell 45, 681-695.e4 (2018).
89. Somerville, T. D. D. et al. Squamous trans-differentiation of pancreatic cancer cells promotes stromal inflammation. Elife 9, (2020).
90. Karamitopoulou, E. Tumour microenvironment of pancreatic cancer: immune landscape is dictated by molecular and histopathological features. British Journal of Cancer vol. 121 5-14 (2019).
91. Sheng, Y. H. et al. MUC13 protects colorectal cancer cells from death by activating the NF-κB pathway and is a potential therapeutic target. Oncogene 36, 700-713 (2017).
92. Skrypek, N. et al. The MUC4 mucin mediates gemcitabine resistance of human pancreatic cancer cells via the Concentrative Nucleoside Transporter family. Oncogene 32, 1714-1723 (2013).
93. Ieta, K., Tanaka, F., Utsunomiya, T., Kuwano, H. & Mori, M. CEACAM6 gene expression in intrahepatic cholangiocarcinoma. Br. J. Cancer (2006) doi:10.1038/sj.bjc.6603276.
94. Roidl, A. et al. Resistance to chemotherapy is associated with fibroblast growth factor receptor 4 up-regulation. Clin. Cancer Res. 15, 2058-2066 (2009).
95. Balaji, S. A., Udupa, N., Chamallamudi, M. R., Gupta, V. & Rangarajan, A. Role of the drug transporter ABCC3 in breast cancer chemoresistance. PLoS One 11, (2016).
96. Niwa, N. et al. TNFAIP2 expression induces epithelial-to-mesenchymal transition and confers platinum resistance in urothelial cancer cells. Lab. Investig. 99, 1702-1713 (2019).
97. Fang, L. et al. Prognostic role of multidrug resistance-associated protein 1 expression and platelet count in operable non-small cell lung cancer. Oncol. Lett. 16, 1123-1132 (2018).
98. Ohkuma, R. et al. High expression of olfactomedin-4 is correlated with chemoresistance and poor prognosis in pancreatic cancer. PLoS One 15, (2020).
99. Shi, X. et al. AQP5 silencing suppresses p38 MAPK signaling and improves drug resistance in colon cancer cells. Tumor Biol. 35, 7035-7045 (2014).
100. Lakshmanan, I. et al. MUC16 regulates TSPYL5 for lung cancer cell growth and chemoresistance by suppressing p53. Clin. Cancer Res. 23, 3906-3917 (2017).
101. Balachandran, V. P. et al. Identification of unique neoantigen qualities in long-term survivors of pancreatic cancer. Nature 551, S12-S16 (2017).
102. Qadir, M. M. F. et al. Single-cell resolution analysis of the human pancreatic ductal progenitor cell niche. Proc. Natl. Acad. Sci. U.S.A 117, 10876-10887 (2020).
103. Swales, N. et al. Plasticity of Adult Human Pancreatic Duct Cells by Neurogenin3-Mediated Reprogramming. PLoS One 7, e37055 (2012).
104. Farrell, A. S. et al. MYC regulates ductal-neuroendocrine lineage plasticity in pancreatic ductal adenocarcinoma associated with poor outcome and chemoresistance. Nat. Commun. 8, 1-12 (2017).
105. Means, A. L. et al. Pancreatic epithelial plasticity mediated by acinar cell transdifferentiation and generation of nestin-positive intermediates. Development 132, 3767-3776 (2005).
106. Storz, P. Acinar cell plasticity and development of pancreatic ductal adenocarcinoma. Nature Reviews Gastroenterology and Hepatology vol. 14 296-304 (2017).
107. Adams, C. R. et al. Transcriptional control of subtype switching ensures adaptation and growth of pancreatic cancer. Elife 8, (2019).
108. Camolotto, S. A. et al. Reciprocal regulation of pancreatic ductal adenocarcinoma growth and molecular subtype by HNF4$α$ and SIX1/4. bioRxiv Cancer Biol. 814525 (2019) doi:10.1101/814525.
109. Sahai, E. et al. A framework for advancing our understanding of cancer-associated fibroblasts. Nature Reviews Cancer vol. 20 174-186 (2020).
110. Cedeno-Laurent, F. et al. Improved pruritus correlates with lower levels of IL-31 in CTCL patients under different therapeutic modalities. Clin. Immunol. 158, 1-7 (2015).
111. Magister, Š. & Kos, J. Cystatins in immune system. Journal of Cancer vol. 4 45(2013).
112. Ullrich, A. et al. The secreted tumor-associated antigen 90K is a potent immune stimulator. J. Biol. Chem. 269, 18401-18407 (1994).
113. Colgan, J., Asmal, M., Yu, B. & Luban, J. Cyclophilin A-Deficient Mice Are Resistant to Immunosuppression by Cyclosporine. J. Immunol. 174, 6030-6038 (2005).
114. Rocha-Perugini, V. et al. CD81 Controls Sustained T Cell Activation Signaling and Defines the Maturation Stages of Cognate Immunological Synapses. Mol. Cell. Biol. 33, 3644-3658 (2013).
115. Zhang, R. et al. Cancer-associated fibroblasts enhance tumor-associated macrophages enrichment and suppress NK cells function in colorectal cancer. Cell Death Dis. 10, 1-14 (2019).
116. Lagares, D. et al. Targeted apoptosis of myofibroblasts with the BH3 mimetic ABT-263 reverses established fibrosis. Sci. Transl. Med 9, (2017).
117. Gunderson, A. J. et al. Blockade of fibroblast activation protein in combination with radiation treatment in murine models of pancreatic adenocarcinoma. PLoS One 14, e0211117 (2019).
118. Chen, I. X. et al. Blocking CXCR4 alleviates desmoplasia, increases T-lymphocyte infiltration, and improves immunotherapy in metastatic breast cancer. Proc. Natl. Acad Sci. U.S.A 116, 4558-4566 (2019).
119. Feig, C. et al. Targeting CXCL12 from FAP-expressing carcinoma-associated fibroblasts synergizes with anti-PD-L1 immunotherapy in pancreatic cancer. Proc. Natl. Acad Sci. U.S.A 110, 20212-20217 (2013).
120. Bockorny, B. et al. BL-8040, a CXCR4 antagonist, in combination with pembrolizumab and chemotherapy for pancreatic cancer: the COMBAT trial. Nat. Med 26, 878-885 (2020).
121. Liu, L. et al. Stromal myofibroblasts are associated with poor prognosis in solid cancers: A meta-analysis of published studies. PLoS One 11, (2016).
122. Cuiffo, B. G. & Karnoub, A. E. Mesenchymal stem cells in tumor development: Emerging roles and concepts. Cell Adhesion and Migration vol. 6 220-230 (2012).
123. Mathew, E. et al. Mesenchymal Stem Cells Promote Pancreatic Tumor Growth by Inducing Alternative Polarization of Macrophages. Neoplasia 18, 142-151 (2016).
124. Dagogo-Jack, I. & Shaw, A. T. Tumour heterogeneity and resistance to cancer therapies. Nature Reviews Clinical Oncology vol. 15 81-94 (2018).
125. Merritt, C. R. et al. Multiplex digital spatial profiling of proteins and RNA in fixed tissue. Nat. Biotechnol. 38, 586⁻⁵⁹⁹ (2020).
126. Monteran, L. & Erez, N. The dark side of fibroblasts: Cancer-associated fibroblasts as mediators of immunosuppression in the tumor microenvironment. Frontiers in Immunology vol. 10 (2019).
127. Vinayak, S. et al. TOPACIO/Keynote-162: Niraparib+pembrolizumab in patients (pts) with metastatic triple-negative breast cancer (TNBC), a phase 2 trial. J. Clin. Oncol. 36, 1011 (2018).
128. Vikas, P., Borcherding, N. & Zhang, W. The clinical promise of immunotherapy in triple-negative breast cancer. Cancer Manag. Res. 10, 6823-6833 (2018).
129. Puleo, F. et al. Stratification of Pancreatic Ductal Adenocarcinomas Based on Tumor and Microenvironment Features. Gastroenterology (2018) doi:10.1053/j.gastro.2018.08.033.
130. Noll, E. M. et al. CYP3A5 mediates basal and acquired therapy resistance in different subtypes of pancreatic ductal adenocarcinoma. Nat. Med. 22, 278-287 (2016).
131. Knudsen, E. S. et al. Pancreatic cancer cell lines as patient-derived avatars: Genetic characterisation and functional utility. Gut 67, 508-502(2018).
132. Sinha, S. et al. PanIN neuroendocrine cells promote tumorigenesis via neuronal cross-talk. Cancer Res. 77, 1868-1879 (2017).
133. Wolock, S. L., Lopez, R. & Klein, A. M. Scrublet: Computational Identification of Cell Doublets in Single-Cell Transcriptomic Data. Cell Syst. 8, 281-291.e9 (2019).
134. Wolf, F. A., Angerer, P. & Theis, F. J. SCANPY: Large-scale single-cell gene expression data analysis. Genome Biol. 19, 15 (2018).
135. Traag, V. A., Waltman, L. & van Eck, N. J. From Louvain to Leiden: guaranteeing well-connected communities. Sci. Rep. 9, (2019).
136. inferCNV of the Trinity CTAT Project.
137. Schapiro, D. et al. HistoCAT: Analysis of cell phenotypes and interactions in multiplex image cytometry data. Nat. Methods 14, 873-876 (2017).
138. Zanotelli, V. R. & Bodenmiller, B. ImcSegmentationPipeline: A pixelclassification based multiplexed image segmentation pipeline. (2017) doi:10.5281/zenodo.3841961.
139. Zanotelli, V., Ndamond & Strotton, M. BodenmillerGroup/ImcSegmentationPipeline: IMC Segmentation Pipeline. (2020) doi:http://doi.org/10.5281/zenodo.3841961.
140. Berg, S. et al. ilastik: interactive machine learning for (bio)image analysis. Nat. Methods 16, 1226-1232 (2019).
141. McQuin, C. et al. CellProfiler 3.0: Next-generation image processing for biology. PLOS Biol. 16, e2005970 (2018).
142. Ramilowski, J. A. et al. A draft network of ligand-receptor-mediated multicellular signalling in human. Nat. Commun. 6, (2015).
143. Subramanian, A. et al. Gene set enrichment analysis: A knowledge-based approach for interpreting genome-wide expression profiles. Proc. Natl. Acad. Sci. U.S.A. 102, 15545-15550 (2005).
144. Li, B. & Dewey, C. N. RSEM: Accurate transcript quantification from RNA-Seq data with or without a reference genome. BMC Bioinformatics 12, (2011).
145. Bates, D., Mächler, M., Bolker, B. & Walker, S. Fitting Linear Mixed-Effects Models Using lme4. J. Stat. Softw. 67, (2015).
146. Kuznetsova, A., Brockhoff, P. B. & Christensen, R. H. B. lmerTest Package: Tests in Linear Mixed Effects Models. J. Stat. Softw. 82, (2017).
147. Benjamini, Y. & Hochberg, Y. Controlling the False Discovery Rate: A Practical and Powerful Approach to Multiple Testing. J. R. Stat. Soc. Ser. B 57, 289-300 (1995).
148. Hanzelmann, S., Castelo, R. & Guinney, J. GSVA: Gene set variation analysis for microarray and RNA-Seq data. BMC Bioinformatics 14, (2013).
149. Liberzon, A. et al. The Molecular Signatures Database Hallmark Gene Set Collection. Cell Syst. 1, 417-425 (2015).
150. Liberzon, A. et al. Molecular signatures database (MSigDB) 3.0. Bioinformatics 27, 1739-1740 (2011).

### Example 2—Single-Nucleus and Spatial RNA Profiling of Frozen Pancreatic Cancer Reveals Multicellular Neighborhoods and Enrichment of a Neuronal-Like Phenotype after Neoadjuvant Treatment

Pancreatic ductal adenocarcinoma (PDAC) is a highly lethal and treatment-refractory cancer. Given the prevalence of resistance to cytotoxic therapy, there is an urgent need to characterize the transcriptional and spatial characteristics of different cell types within and across tumors, particularly in residual disease. Here, Applicant developed a robust single-nucleus RNA-seq technique for banked frozen primary tumor specimens, applied it to 43 independent tumors that either received neoadjuvant therapy or were treatment-naive, and integrated these findings with whole-transcriptome digital spatial profiling (DSP) to provide a high-resolution landscape of the multicellular subtypes and spatial communities that compose PDAC. Applicant learned expression programs across malignant cell and fibroblast profiles and uncovered a refined molecular taxonomy, including a newly-identified neuronal-like malignant cell program. Neoadjuvant therapy was associated with a depletion in classical-like and squamoid phenotypes and an enrichment in mesenchymal, neuroendocrine-like, and neuronal-like malignant programs, with the neuronal-like program associated with the worst prognosis in bulk profiles from prior independent cohorts. Spatial profiling mapped cell types and programs onto the tumor architecture to reveal three multicellular microcommunities, which Applicant annotated as classical, squamoid-basaloid, and treatment-enriched. Opportunities to improve neoadjuvant and adjuvant therapy for PDAC may arise from the observed enrichment of a module featuring the neuronal-like malignant program; neurotropic CAF program; and CD8+ T cells as well as CXCL12-CXCR4, CXCL3 CXCL5-CXCR2, CCL19-CCR7, TNFSF15-TNFRSF25, ILIA-ILIR1, IL33-ILIR1, IL10-IL10RA, and ERBB2 interactions in post-treatment residual disease. Our refined molecular taxonomy coupled with spatial resolution and insights into treatment-associated reprogramming may help advance precision oncology strategies in PDAC through informative stratification in clinical trials and provide a roadmap for therapeutic targeting based on multicellular phenotypes and interactions.

Pancreatic ductal adenocarcinoma (PDAC) is projected to become the second leading cause of cancer death in the United States within the next two to three decades^{1,2}. Despite advancements in systemic therapy, tumor resistance to cytotoxic therapy remains a profound challenge for PDAC. There is an urgent need to understand how preoperative treatment enriches for and impacts residual cancer cells and stroma to identify additional therapeutic vulnerabilities that can be exploited in combination with or after neoadjuvant chemotherapy and/or radiotherapy^{3,4}.

Unlike many other common cancers, molecular subtyping of pancreatic cancer remains in its nascent stages and does not currently inform clinical management or therapeutic development^{5,6}. RNA profiling of bulk PDAC tumors⁷⁻¹² identified two consensus subtypes: (1) classical/epithelial, encompassing a spectrum of pancreatic lineage precursors, and (2) basal-like/squamous/quasi-mesenchymal, characterized by loss of endodermal identity and genetic aberrations in chromatin modifiers⁵. While the basal-like, squamous, and quasi-mesenchymal subtypes have generally been thought of as a single consensus classification in the literature^{5,13}, there are indications that there may be non-overlapping characteristics among them. For example, when comparing the basal-like subtype⁸ to the quasi-mesenchymal subtype⁷, the latter was purported to be significantly influenced by genes that may derive from stromal contamination¹⁰. Basal-like tumors were associated with worse survival and poorer responses to chemotherapy¹⁴, but attempts to refine this classification (beyond basal-like vs. classical) have failed to further stratify patient survival^{5,9}. Additional proposed subtypes, such as exocrine, aberrantly-differentiated endocrine exocrine (ADEX), and immunogenic, are associated with lower tumor purity and have been postulated to characterize stromal contributions rather than neoplastic cell-intrinsic programs^{9,10}. Bulk profiling studies attempted to address this challenge by enriching for neoplastic content through specimen selection or microdissection⁷⁻¹⁰, but these introduced sampling bias and still precluded the assignment of transcripts to individual cells. Thus, it remains unclear if genes expressed in ‘normal’ differentiated pancreatic tissue have a role in cancer or represent non-malignant tissue contamination. Furthermore, most studies have been performed in the untreated setting and do not offer guidance for treatment approaches after neoadjuvant therapy.

Distinguishing the relationships among malignant and non-malignant cells in the tumor microenvironment (TME) is also important because the therapeutic effect of cytotoxic treatments may be significantly modulated by cancer-associated fibroblasts (CAFs) and the immune system¹-1⁷. These observations have motivated the deployment of angiotensin system inhibitors such as losartan to reduce stromal collagen and hyaluronan, resulting in increased vascular perfusion, enhanced T cell and antigen presenting activity, and improved chemotherapy and oxygen delivery to tumors—the latter of which is known to potentiate the efficacy of radiotherapy²⁶-2⁹. A phase 2 trial of patients with locally-advanced PDAC receiving a total neoadjuvant approach comprised of FOLFIRINOX (5-fluorouracil, oxaliplatin, irinotecan, leucovorin) and losartan followed by chemoradiotherapy resulted in favorable outcomes compared to historical controls⁴. These promising results have inspired an ongoing randomized phase 2 trial for patients with locally-advanced PDAC to examine the impact of adding neoadjuvant losartan and/or the anti-PD-1 inhibitor nivolumab to a backbone of FOLFIRINOX chemotherapy and stereotactic body radiotherapy (NCT03563248).

Single-cell RNA-seq (scRNA-seq) is optimally positioned to distinguish the diversity of malignant and non-malignant cells in the tumor³⁰-3⁴, and elucidate the impact of therapy on each cellular compartment and their interactions. However, scRNA-seq in PDAC has lagged behind other cancer types, due in part to high intrinsic nuclease content and dense desmoplastic stroma³⁵⁻³⁸, compromising RNA quality, cell viability, capture of malignant and stromal cells, and compatibility with treated tumors. Single-nucleus RNA-seq (snRNA-seq) provides a compelling alternative for difficult-to-dissociate specimens or frozen banked samples³⁹⁻⁴², and can often better recover malignant cells, stroma, and their native cell states, while reducing stress signatures (introduced by dissociation)⁴³⁻⁴⁵. However, snRNA-seq has not yet been successfully applied to PDAC tissues previously, and like scRNA-seq, is unable to provide the spatial context offered by complementary—albeit lower resolution—spatial transcriptomic methods⁴⁶.

Here, Applicant optimized snRNA-seq for banked frozen PDAC specimens stored for up to five years, profiled 224,988 nuclei across 18 tumors from untreated patients and 25 tumors from those who received neoadjuvant treatment prior to resection, and achieved comparable quality for untreated and treated tumors. The recovered cellular composition was similar overall to that determined by a histological standard of multiplex protein profiling in situ. Applicant discovered treatment-associated remodeling of the malignant, fibroblast, and immune compartments; identified changes in expression programs in malignant cells and fibroblasts associated with treatment; and refined the molecular taxonomy of PDAC in a clinically relevant manner. By mapping these expression programs using spatially resolved mRNA profiles, Applicant associated neoplastic- and fibroblast-intrinsic programs with various immune cell types/states to uncover distinct communities in the PDAC microenvironment and identified neighborhood and intercellular interactions enriched in post-treatment residual disease. Our work provides a high-resolution window into treatment selection pressures and associated changes in the molecular composition of tumors, and offers a blueprint for exploring therapeutic strategies tailored for the reprogrammed tumor.

### Single-Nucleus RNA-Seq Accurately Represents the Malignant and Non-Malignant Compartments of Human PDAC Tumors

Of the 50 patients enrolled in this excess tissue study, Applicant performed snRNA-seq on flash frozen, histologically-confirmed, primary PDAC specimens from 43 patients who underwent surgical resection with (n=25) or without (n=18) neoadjuvant treatment (FIG. 17A;), and analyzed 224,988 high quality single nucleus profiles (Methods). Most treated patients had received multiple cycles of cytotoxic chemotherapy (FOLFIRINOX) followed by multi-fraction radiotherapy with concurrent 5-FU or capecitabine (CRT; n=14). Seven additional patients were enrolled on therapeutic interventional clinical trials (NCT03563248, NCT01821729) investigating neoadjuvant CRT with the addition of losartan (CRTL; n=5); nivolumab (CRTN; n=1); or both (CRTLN; n=1); another four patients received other forms of neoadjuvant.

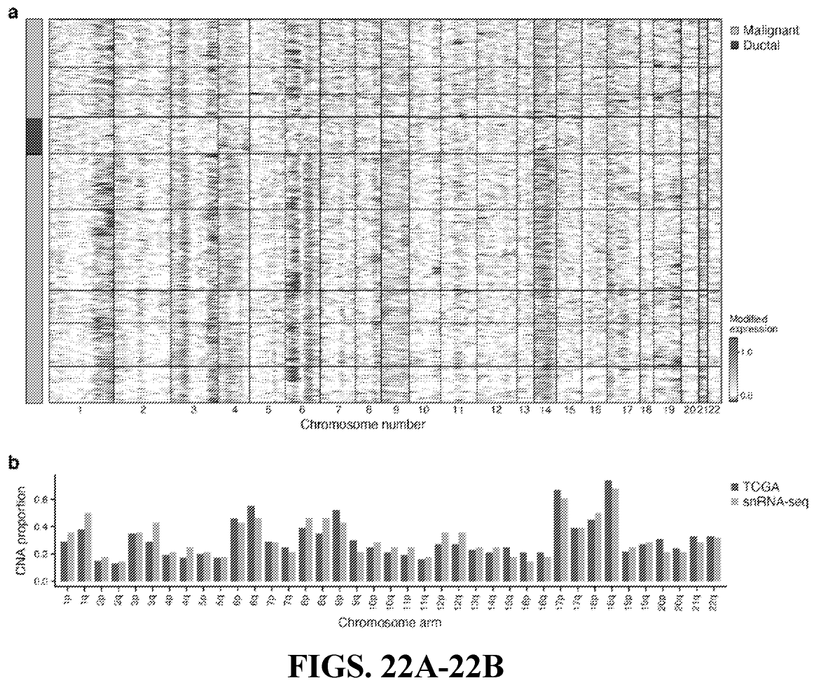

Applicant performed unsupervised clustering of single nucleus profiles and annotated cell subsets using known cell type-specific gene signatures (FIGS. 17B-17D; Methods)^{42,47-50}. The identity of malignant cells was confirmed by inferred Copy Number Alterations (CNAs) (FIG. 22A)³⁰. The frequencies and distributions of inferred chromosome arm-level somatic CNAs in the malignant cells of these patient samples were comparable to those derived from The Cancer Genome Atlas (TCGA) pancreatic adenocarcinoma cohort as assessed by single nucleotide polymorphisms and whole-exome sequencing (FIG. 22B)¹⁰. There was marked heterogeneity in cell type distribution across tumors (FIGS. 21A-21B). Additionally, Applicant observed the malignant cells to be partitioning into distinct clusters by patient measured by the adjusted mutual information (AMI) score of 0.87 in malignant cells compared to 0.18 in non-malignant cells. Beyond the epithelial compartment, Applicant found a diverse range of cell types known to compose exocrine pancreatic tumors including immune, endocrine, and other stromal cells such as CAFs, endothelial cells, vascular smooth muscle, pericytes, intra-pancreatic neurons, Schwann cells, and adipocytes (FIGS. 17B, 17D).

To assess if our method captured representative cell type proportions, Applicant compared it to estimates from Multiplexed Ion Beam Imaging (MIBI). Applicant applied a 27-plex epithelial oncology panel to formalin-fixed paraffin-embedded (FFPE) sections derived from tumor specimens in a subset of seven individuals (FIG. 17A; Methods)^{51,52}. Quantification of broad cell types revealed that snRNA-seq captures a representative distribution of epithelial, fibroblast, endothelial, and immune cells present in PDAC, across all tumors (FIG. 17E), and within individual tumors (FIG. 24 While the broad compartments were in good agreement, within immune cell subsets (Methods), there were greater differences between snRNA-seq and MIBI (FIG. 17E; FIG. 24), suggesting differential capture of different immune cell subsets⁴⁵. For example, B and plasma cells in the treated samples (but not the untreated samples) were identified in situ but under-represented by snRNA-seq whereas a higher proportion of macrophages was captured by snRNA-seq regardless of treatment status (FIG. 17E, FIG. 24).

While earlier scRNA-seq studies in PDAC did not fully capture the stromal milieu and necessitated enrichment strategies for CAFs such as fluorescence-activated cell sorting⁵³⁻⁵⁶, they are well-represented in our samples ((FIGS. 17B, 17D; FIGS. 21A-21B). Specifically, our snRNA-seq had a higher yield of high quality nuclei per patient in the untreated group (6,054±1,529) than a recent scRNA-seq study of primary untreated PDAC⁵⁶ (1,718±773), despite comparable quantities of loaded cells/nuclei (p=1.92×10⁻⁹, Mann-Whitney U test; Methods; FIG. 23), recovered six additional cell types absent in scRNA-seq, and captured significantly higher proportions of CAFs, pericytes, and endocrine cells and lower proportions of vascular smooth muscle cells, myeloid cells, lymphoid cells, and endothelial cells (p<0.05; Mann-Whitney U Test; comparable results using Dirichlet-multinomial regression; FIG. 23).

Epithelial Cell Profiles Suggest that Acinar to Ductal Metaplasia (ADM) and Atypical Ductal Cells are Intermediate States in PDAC Development

Within the epithelial compartment, Applicant also identified the presence of low CNA nuclei co-expressing markers of ductal and acinar lineages (FIGS. 17B, 17D) with the number of unique molecular identifiers typical for single nuclei. These may represent the putative acinar-to-ductal metaplasia (ADM) that has been shown to play an initiating role in mouse pancreatic tumorigenesis, though direct evidence for ADM as a route of malignant transformation in humans is inadequate^{57,58}. Chronic inflammation and somatic KRAS mutations have been linked to persistence of the ADM state and progression to pre-invasive lesions (e.g., pancreatic intraepithelial neoplasia or PanIN)^{58,59}. Consistent with these prior observations, the epithelial cells annotated as ADM exhibit greater expression of the HALLMARK_KRAS_SIGNALING_UP signature⁶⁰ compared to acinar cells (FIG. 17F). Notably, a small subset of acinar cells (acinar-REG+) expressed high levels of regenerating family member genes (e.g., REG1A, REG3A), which have been implicated in promoting pancreatic inflammation, ADM and PanIN (FIG. 17D)^{42,48,61,62}. Moreover, a distinct subset of ductal cells featured markers of both ductal (e.g., CFTR) and malignant (e.g., KRT5, KRT17, KRT19) cells without elevated CNAs, which Applicant termed atypical ductal cells. The atypical ductal cell population featured genes (e.g., KRT17)⁶³ that are expressed as early as the PanIN2/3 stage and had higher levels of the HALLMARK_KRAS_SIGNALING_UP signature relative to the ADM population, suggesting that the atypical ductal cells may represent progression from ADM to precursor lesions such as PanINs (FIGS. 17B, 17D, 17F). Supporting this interpretation, a partition-based graph abstraction (PAGA) that estimates the connectivity of manifold partitions⁶⁴ inferred a dominant pseudotemporal trajectory from acinar to ADM to ductal to atypical ductal to malignant cells (FIG. 17G). This inferred trajectory combined with a parallel monotonic increase in the HALLMARK_KRAS_SIGNALING_UP signature expression lends support to the notion that ADM and atypical ductal cells are likely relevant intermediate states in the clinical development of PDAC (FIG. 17F-17G).

### Alterations in Tumor Composition Following Neoadjuvant Treatment Reveal Increased Abundance of Cytotoxic T Cells and Decreased Prevalence of Regulatory T Cells

Applicant compared the snRNA-seq cell type proportions across our clinical subgroups, focusing on those with at least five patients (untreated, CRT, CRTL) (FIGS. 18A and 25). Consistent with treatment effect and confirmed by histology, the proportion of malignant cells was significantly lower in tumors treated with neoadjuvant therapy (CRT vs. untreated, p=3.87×10⁻⁴; CRTL vs. untreated, p=1.27×10⁻³; Mann-Whitney U test; also significant when accounting for the interdependence of proportions with inclusion probability pᵢ>0.99 using Dirichlet-multinomial regression) (FIG. 18A). The proportions of several non-malignant cell types also varied across treatment groups (FIGS. 18A and 25), but only the lymphoid (pᵢ=0.795; Dirichlet-multinomial regression) and myeloid (pᵢ=0.826) populations were significantly higher in CRTL vs. untreated after adjusting for the interdependence of proportions.

Within the immune compartment, neoadjuvant CRTL was associated with a higher fraction of CD8+ T cells compared to untreated (p=3.34×10⁻²; Mann-Whitney U test) and CRT (p=1.17×10⁻²) (FIGS. 18A and 25). In contrast, regulatory T cells (T_{regs}) were present in lower proportions for both treatment groups (CRT vs. untreated, p=7.56×10⁻³; CRTL vs. untreated, p=3.49×10⁻²; Mann-Whitney U test). Differential gene expression analysis between treatment groups using patient ID as a random effect (FIG. 26; Methods) revealed that CD8+ T cells in the CRTL tumors also exhibited greater effector function (e.g., IL2, CCL4, CCL5)^{65,66} and reduced expression of quiescence and dysfunction markers (e.g., TIGIT, TCF7, KLF2, LEFI)⁶⁷⁻⁷³ compared with untreated and CRT-treated tumors. These results are consistent with the previously identified link between losartan-mediated increase in intra-tumoral cytotoxic T cell activity with concomitant reduction in T_{regs}^{28,74}

### Learning Shared Expression Programs in Malignant Cells and Fibroblasts Across Tumors

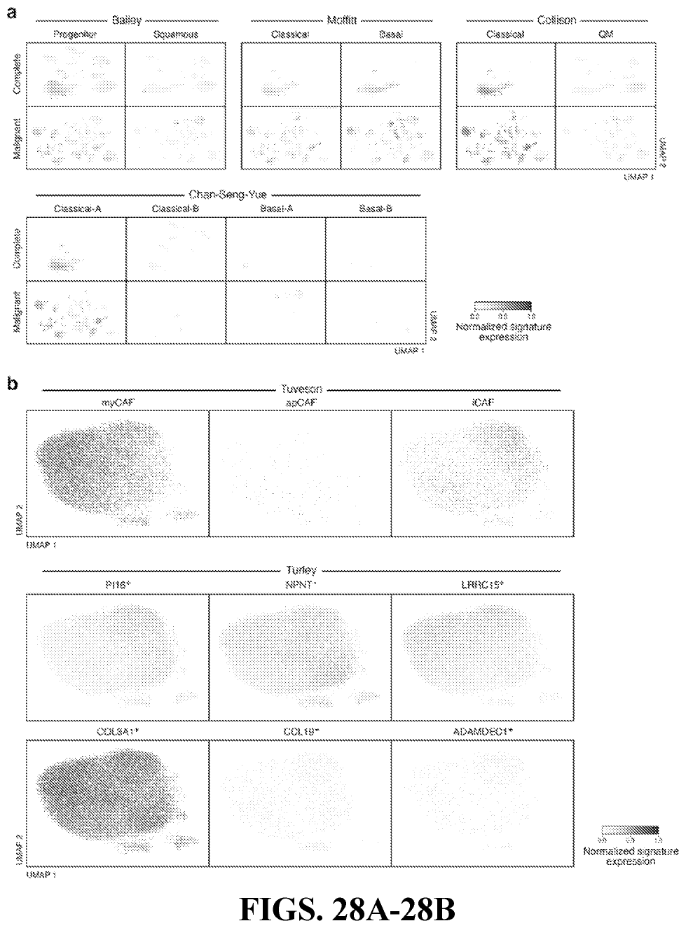

Next, Applicant sought to better characterize expression states within the malignant cells and CAFs across patients. Consistent with recent reports of intra-tumoral subtype heterogeneity, most tumors contained both basal-like/squamous/quasi-mesenchymal and classical/epithelial cells (FIG. 28A; Methods)^{75,76}, but these states (defined from bulk profiles) overlapped in some cells while other cells did not strongly express either signature. This suggests signature promiscuity, dynamic transitions, the existence of “hybrid” cells^{76,77}, and/or previously unidentified phenotypes. The myofibroblastic CAF (myCAF) and inflammatory CAF (iCAF) signatures were expressed in somewhat distinct subsets of CAFs in our cohort, but the antigen-presenting (apCAF) signature was not clearly identified in our data (FIG. 28B; Methods)^{54,78}. In addition, cross-tissue fibroblast lineage signatures (e.g., COL3A1⁺ myofibroblast, LRRC15⁺ myofibroblast, CCL19⁺ colitis, ADAMDECI⁺ colitis, NPNT⁺ alveolar, and PI16⁺ adventitial) were moderately capable of segregating our CAF snRNA-seq data (FIG. 28B).

To address these challenges, Applicant pursued a de novo approach, by learning recurrent expression programs across malignant cells and CAFs of different tumors, using consensus non-negative matrix factorization (cNMF)⁷⁹. Applicant focused on programs shared among multiple patients that were biologically distinct (FIG. 18B; Methods) and annotated each program based on its top-200 weighted genes (Methods).

### Refined and Expanded Malignant Cell Classification Highlights Seven Lineage States, Including a Novel Neuronal-Like State

For malignant cells, Applicant identified 14 programs that reflected either lineage (classical, squamoid, basaloid, mesenchymal, acinar-like, neuroendocrine-like, neuronal-like) or cell state (cycling (S), cycling (G2/M), MYC, interferon, TNF-NFκB, ribosomal, adhesive) (FIG. 18B).

While the classical-like program strongly overlapped with previously defined classical signatures^{8,76} [Moffitt classical subtype (p=8.12×10⁻¹¹; hypergeometric test), classical A subtype (p=5.61×10⁻⁶⁶), classical B subtype (p=1.00×10⁻¹⁰)], Applicant also identified three other distinct programs corresponding to squamoid, basaloid, and mesenchymal, in lieu of a joint basal-like/squamous/quasi-mesenchymal program⁷⁻⁹ captured by bulk studies. Both the squamoid and basaloid programs overlapped significantly with the Moffitt basal-like signature (p=3.72×10⁻⁹ and 1.93×10⁻³, respectively)⁸, but the squamoid and mesenchymal programs did not exhibit significant overlap with the bulk-defined squamous subtype⁹ and quasi-mesenchymal subtype⁷, respectively. Further substantiating our nomenclature, the squamoid program was enriched in genes (GSEA) that play a role in epidermis development/proliferation, keratinocyte differentiation, and cornification (e.g., KRT13, KRT16, SCEL)⁸⁰⁻⁸², which are associated with the suprabasal layers of the epidermis, and genes downregulated in breast cancer cells exhibiting epithelial-mesenchymal transition (EMT) (FIG. 18B). In contrast, the basaloid program was enriched in genes associated with stemness, ribosome proteins and rRNA processing, neurogenesis, cell migration, tumor invasiveness, cell-cell and cell-extracellular matrix (ECM) junctions, and EMT (FIG. 18B). Analogously, benign prostatic basal epithelial cells preferentially express genes associated with stemness, neurogenesis, and rRNA biogenesis compared to luminal epithelial cells⁸³. Moreover, the basaloid program included several heavy metal-binding metallothioneins (e.g., MT1A, MT1E, MT2A) whose elevated expression in basal cells has been implicated in epidermal hyperplasia⁸⁴. Finally, the mesenchymal program featured strong enrichment for genes involved in EMT, matrisome, ECM production, and stemness (FIG. 18B)^{85,86}.

Our analysis further revealed the existence of neuroendocrine-like and acinar-like programs in high CNA bona fide malignant cells (FIG. 18B), the former of which is reminiscent of neuroendocrine-like differentiation states observed in other epithelial malignancies including prostate, bladder, and non-small cell lung cancer⁸⁷⁻⁸⁹. While prior bulk studies have suggested the possibility of endocrine-like or exocrine-like expression phenotypes, they were often ascribed to non-malignant cells because of the lower tumor purity scores associated with these subtypes^{5,7,9,10}. Our analysis shows that these are present in malignant cells.

Finally, Applicant uncovered a novel neuronal-like program that was enriched for pathways involved in neuron development/differentiation and axon guidance but not marked endocrine lineage features, distinguishing it from the neuroendocrine-like program (FIG. 18B). This program features secreted factors with neuromodulatory roles (e.g., RELN, SEMA3E, NRP1, NRG1)⁹⁰⁻⁹³ and is reminiscent of studies exhibiting the impact of tumor-nerve crosstalk on tumor initiation, growth, invasion, dissemination, post-treatment recurrence, and pain, as well as those detailing frequent and diverse somatic aberrations in genes linked to axonal guidance^{94,95}. Supporting this role, the neuronal-like malignant program (but not the neuroendocrine-like program) is significantly enriched (p=0.006; Kolmogorov-Smirnov test) in genes that are differentially expressed between untreated PDAC tumors with (n=134) or without (n=25) perineural invasion (PNI) by histologic assessment⁹⁶ (Methods, FIG. 29). A neuronal subtype featuring genes involved in neuronal differentiation has been described in other malignancies such as bladder cancer and is associated with a poor prognosis^{88,97}, but has been difficult to distinguish from neuroendocrine-like histology and expression^{98,99}. Our single-nucleus resolution appeared to decouple these two programs (FIG. 18C).

Applicant also identified seven additional ‘cell state’ programs in malignant cells (FIG. 18B). These spanned cell cycle programs in S and G2/M phases; a ribosomal program enriched in genes associated with ribosome components, translation, and protein targeting to the membrane; an interferon program enriched in type 1 and 2 interferon response genes and other cytokines; a TNF-NFkB program with targets of the TNF-α and NF-κB signaling pathways; a MYC program with targets of MYC signaling; and an adhesive program with genes important in cell adhesion (e.g., CDH1) and motility (FIG. 18B). Inter-program correlation scores showed strong associations between the ribosomal and basaloid; MYC and classical; adhesive and neuronal-like; and among the cycling (S), cycling (G2/M), and MYC programs, some of which is consistent with prior works^{83,100-102} (FIGS. 30A-30B).

### Myofibroblastic-Progenitor, Neurotropic, Immunomodulatory, and Adhesive CAF Expression Programs

Among CAFs, Applicant identified four distinct programs which Applicant termed: myofibroblastic-progenitor, neurotropic, immunomodulatory, and adhesive (FIG. 18B). Comparisons to previously-described CAF signatures revealed that the ACTA2-enriched myofibroblastic-progenitor program significantly overlapped with a single-cell myCAF signature (p=1.04×10⁻¹²; hypergeometric test)⁵⁴ (FIG. 18B) but was differentiated by an enrichment of genes involved in embryonic mesodermal development and Wnt signaling (e.g., RUNX1, RUNX2, LEF1, SALL4, WNT5A, NKD1, FOXP1)¹⁰³⁻¹¹⁰. The neurotropic, immunomodulatory, and adhesive programs all overlapped with the single-cell iCAF (p=1.90×10⁻¹⁰, 3.22×10⁻¹⁴, and 3.85×10⁻⁴, respectively; hypergeometric test) but not the myCAF (p=0.149, 0.851, and 0.851, respectively) signatures, suggesting they may reflect different iCAF subsets. None of the four CAF programs identified had significant overlap with the single-cell apCAF signature⁵⁴. In addition, cross-tissue fibroblast clusters defined in the context of inflammatory, fibrotic, and malignant conditions in the lung, colon, and pancreas¹¹¹ had significant concordance with our cNMF programs. For example, the myofibroblastic progenitor program had significant overlap with the LRCC15⁺ myofibroblast (p=8.71×10⁻⁴²; hypergeometric test) and COL3A1⁺ myofibroblast (p=2.86×10⁻¹³) clusters; the neurotropic program with the PI16⁺ adventitial (p=7.65×10⁻²⁴) and NPNTP⁺ alveolar (p=1.62×10⁻³⁷) clusters; the immunomodulatory program with the CCL19⁺ colitis (p=1.30×10⁻²⁹) and ADAMDECI⁺ colitis (p=5.28×10⁻¹²) clusters; and the adhesive program with the PI16⁺ adventitial (p=2.58×10⁻⁵¹) and LRRC15⁺ myofibroblast (p=1.73×10⁻¹²) clusters.

Further examination of our CAF programs independent of prior signatures revealed that the immunomodulatory program was enriched in pathways involving cytokine production/response, inflammation, and TNF-α/NF-κB signaling. Specifically, this program included CXCL12, which recruits lymphocytes and monocytes but has an overall immunosuppressive and protumorigenic role in pancreatic cancer^{112,113}; CCL19 and CCL21, which are chemoattractants for CCR7-expressing lymphocytes and play other roles in the pathogenesis of pancreatic cancer^{114,115}; IL34, which induces proliferation and differentiation in monocytes and macrophages¹¹⁶; and several members of the complement pathway (e.g., C3, C7, CR1, and CR2), which may play a role in neutrophil recruitment among other effects^{117,118} (FIG. 18B). The adhesive program featured pathways involved in cell-cell/cell-ECM adhesion (e.g., CDH2), cytoskeletal remodeling, and motility. The neurotropic program was enriched for genes involved in neurogenesis, neuron differentiation, and neuronal projections (FIG. 18B). CAFs have been linked directly and indirectly to neurotropic phenomenon in pancreatic cancer¹¹⁹.

Treatment-Associated Patterns in Malignant and CAF Programs Provide Insights into Potential Resistance Programs

Neoadjuvant therapy was associated with significant differences in the pattern and prevalence of malignant and CAF programs at the patient level (FIG. 18D; FIG. 31; Methods). The malignant classical (p=4.44×10⁻⁴; Mann-Whitney U test) and squamoid (p=1.11×10⁻³) programs were significantly lower in CRT vs. untreated, whereas the neuroendocrine-like (p=4.63×10⁻³) and neuronal-like (p=2.33×10⁻³) programs were higher (FIG. 18D; FIG. 31). Neoadjuvant CRTL had higher expression of neuronal-like (vs. untreated, p=7.68×10⁻⁴; Mann-Whitney U Test) and mesenchymal (vs. untreated, p=1.24×10⁻¹) programs and lower expression of the classical (vs. untreated, p=2.59×10⁻³), squamoid (vs. untreated, p=4.55×10⁻⁴; vs. CRT, p=3.55×10⁻²), basaloid (vs. untreated, p=5.06×10⁻³; vs. CRT, p=4-33×10⁻²), and acinar-like (vs. untreated, p=2.87×10⁻²; vs. CRT, p=3.55×10⁻²) programs (FIG. 18D; FIG. 31).

Neoadjuvant treatment was associated with lower expression of cycling programs (S: CRT vs. untreated, p=3.27×10⁻²; CRTL vs. untreated, p=2.02×10⁻² and G2/M: CRT vs. untreated, p=2.94×10⁻³; CRTL vs. untreated, p=2.02×10⁻²; Mann-Whitney U test). Furthermore, CRTL was associated with higher expression of the adhesive program (p=1.11×10⁻⁵; Mann-Whitney U test) and lower expression of the ribosomal program (p=3.39×10⁻²) compared to untreated samples (FIG. 18D; FIG. 31). In the CAF compartment, treatment was associated with lower myofibroblastic-progenitor program expression (CRT vs. untreated, p=5.08×10⁻⁴; CRTL vs. untreated, p=1.62×10⁻³; Mann-Whitney U test) and higher adhesive program expression (CRT vs. untreated, p=6.42×10⁻³; CRTL vs. untreated, p=9.9×10⁻⁴; CRTL vs. CRT, p=1.17×10⁻²) (FIG. 18D; FIG. 31). Both the immunomodulatory and the neurotropic CAF programs were higher in CRT (immunomodulatory: CRT vs. untreated, p=1.75×10⁻²; CRT vs. CRTL, p=3.16×10⁻³; neurotropic: CRT vs. untreated, p=3.00×10⁻², Mann-Whitney U test). These differences were overall consistent with differential gene expression among untreated, CRT, and CRTL samples (FIGS. 27 and 32; Methods; linear mixed-effects model with patient ID as a random effect).

Irrespective of the treatment regimen, Applicant further annotated each of the 25 treated samples based on the patient's surgical pathology treatment response grade (poor, minimal, or moderate), and scored their remaining (residual) malignant cells for the seven malignant lineage programs. The residual malignant cells in patients with moderate response were enriched in the neuronal-like program and depleted in the classical-like and squamoid programs relative to the more abundant malignant cells in untreated tumors (p<0.01, Mann-Whitney U test) and those exhibiting poor response to treatment (p<0.08) (FIG. 33). Notably, the neuronal-like program mean normalized score monotonically increased from untreated to poor response to minimal or moderate response, whereas classical-like and squamoid programs monotonically decreased in this same order (FIG. 33).

Neuronal-Like Malignant Program is Associated with Poor Clinical Outcomes

To assess the potential prognostic relevance of the malignant cell and CAF programs¹²⁰, Applicant scored them in bulk RNA-seq data from clinically-annotated patients in TCGA¹⁰ and the PanCuRx^{14,76} cohorts with untreated, resected primary PDAC (n=269), annotated by age, sex, stage, grade, overall survival (OS, 167 deaths), and time to progression (TTP, 154 progression events) (Methods). Applicant performed a multivariable Cox regression analysis of the TTP and OS endpoints with age, sex, stage, and grade and all the CAF and malignant cell programs as covariates. Age, sex, stage, and grade were not prognostic for TTP (FIG. 18E).

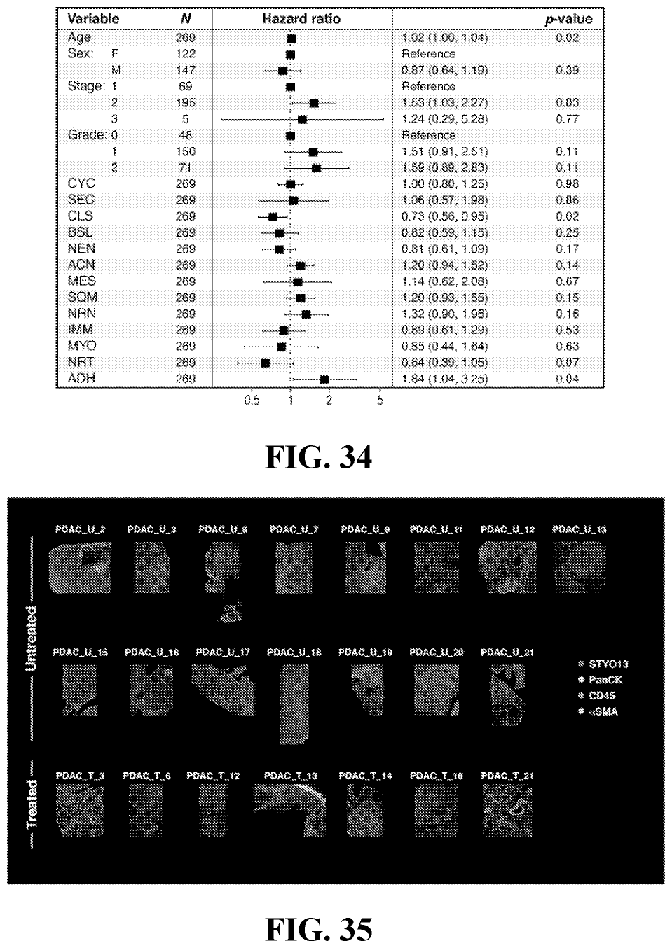

The classical-like malignant program (HR 0.61, 95% CI: 0.46-0.82) and immunomodulatory CAF program (HR 0.59, 95% CI: 0.39-0.89) were significantly associated with longer TTP, whereas the neuronal-like (HR 1.62, 95% CI: 1.08-2.42) and squamoid programs (HR 1.35, 95% CI: 1.02-1.78) were significantly associated with a shorter TTP (FIG. 18E). For OS, age (HR 1.02, 95% CI: 1.00-1.04) and the adhesive CAF program (HR 1.84, 95% CI: 1.04-3.25) were significantly correlated with shorter survival whereas the classical-like malignant program (HR 0.73, 95% CI: 0.56-0.95) was significantly associated with longer survival. The neuronal-like program (HR 1.32, 95% CI: 0.90-1.96) and squamoid program (HR 1.20, 95% CI: 0.93-1.55) trended towards a negative association with OS but did not reach significance (FIG. 34). An important caveat of applying our snRNA-seq programs to bulk transcriptomic data is confounding by the presence of non-malignant cell types that may express program genes to varying degrees.

High-Resolution Mapping of Cell Types and Gene Expression Programs onto Tumor Architecture by Spatial mRNA Profiling

Our snRNA-seq analysis provided a detailed window into the diversity and clinical relevance of expression programs in malignant cells and CAFs in untreated and treated tumors, but could only indirectly infer associations among malignant, stromal and immune cells. Uncovering how malignant cell- and fibroblast-intrinsic programs modulate the composition of local microniches remains an open question in PDAC research and will be critical in guiding therapeutic and biomarker development^{10,121,122}.

To address this question, Applicant performed digital spatial profiling (DSP) with the GeoMx platform (NanoString) using a human whole transcriptome atlas (WTA; 18,269 mRNA targets). In this method, UV-photocleavable barcode-conjugated RNA ISH probes were hybridized on FFPE tissue sections and used to capture and profile mRNA counts from user-defined regions of interest (ROIs) (FIGS. 19A and 35; Methods)¹²³. A four-color immunofluorescence slide scan for each specimen showed intra-tumoral heterogeneity in tissue architecture and ROIs with diverse patterns of neoplastic cells, CAFs, and immune cells were selected for profiling (FIGS. 19A and 35; Methods). Applicant then created custom masks or areas of illumination (AOI) for each cell type segment within the ROI, cleaved and collected barcodes from each AOI in a spatially-indexed manner, and quantified barcode abundance by sequencing (Methods).

Applicant analyzed 21 tumors by DSP whole transcriptome analysis, 18 of which were also profiled by snRNA-seq (FIGS. 17A, 19A, and 35; Methods)¹²³. Applicant deconvolved the WTA data with our snRNA-seq cell type signatures. The epithelial, CAF, and immune AOIs clustered appropriately by cell type, demonstrating the coherence and complementarity of the two platforms (FIG. 36). Next, Applicant used the WTA data to map the expression of each malignant and CAF program onto the ROIs (FIG. 19B), with 54% of the top 200-weighted program genes detected above background (Methods).

Overall, there was greater spatial heterogeneity in the expression of most snRNA-seq programs across tumors than across ROIs within a tumor, except for the mesenchymal malignant and immunomodulatory and myofibroblastic progenitor CAF programs (FIG. 19B). Only the neuronal-like and neuroendocrine-like programs were enriched in ROIs from CRT vs. untreated samples (p=1.07×10⁻², 4.98×10⁻², respectively; linear mixed-effects model with patient ID as random effect; FIG. 37), consistent with their enrichment in the snRNA-seq data (FIG. 18D).

### Spatial Association Among Malignant Programs, Fibroblast Programs and Immune Cell Composition in Tumors Defines Distinct Neighborhoods

To investigate potential spatial associations among cells of different types or programs, Applicant constructed a ROI-based feature correlation matrix composed of the seven malignant lineage programs (FIG. 18B); four CAF programs (FIG. 18B); proportions of immune cell types by deconvolution using snRNA-seq signatures (Methods); and the percent ROI area occupied by the malignant, fibroblast, and immune segments (FIG. 19C). Unsupervised hierarchical clustering was performed using the Pearson correlation distance and average linkage (FIG. 19C; Methods). The “treatment-enriched” neighborhood 1 was characterized by a strong association among the neuronal-like and neuroendocrine-like malignant programs, the neurotropic CAF program, and CD8⁺ T cells, as well as a weaker relationship among the mesenchymal and acinar malignant programs and the immunomodulatory CAF program (FIGS. 19C, 19D). The “squamoid-basaloid” neighborhood 2 featured an association of squamoid and basaloid malignant programs with a diverse set of immune cells including CD4⁺ T cells, T_{regs}, natural killer cells, conventional type 1 dendritic cells, activated dendritic cells, plasmacytoid dendritic cells, B cells, plasma cells, and mast cells (FIGS. 19C, 19D). The “squamoid-basaloid” neighborhood was also positively correlated with % epithelial area and % immune area but negatively correlated with % CAF area. Although % immune area clustered with neighborhood 2, Applicant also noted a consistent positive correlation between the features of neighborhood 1 and % immune area (FIGS. 19C, 19D). Lastly, the “classical” neighborhood 3 exhibited an association among the classical malignant program, the myofibroblastic progenitor and adhesive CAF programs, and macrophages, neutrophils, and conventional type 2 dendritic cells (FIGS. 19C, 19D). The “classical” neighborhood was positively correlated with % CAF area and negatively associated with % immune area. In parallel, Applicant constructed a patient-level feature correlation matrix using the dissociated snRNA-seq data and performed unsupervised hierarchical clustering (FIG. 38). Some observed associations in the DSP analysis (FIGS. 19C, 19D) were recapitulated in the snRNA-seq analysis, including co-clustering of the classical malignant program with myofibroblastic progenitor CAFs; squamoid and basaloid malignant programs; and neuronal-like malignant program with neurotropic CAF program and CD8⁺ T cells while other patterns were different (FIG. 38), which is not surprising given the lack of spatial resolution in the snRNA-seq data.

Clustering of features in a combined correlation matrix provides an excellent overview of spatial neighborhoods in PDAC, but certain individual features within clusters may not be strongly correlated. For example, the classical malignant program was anti-correlated with macrophage and neutrophil prevalence despite co-localizing to the same cluster (FIG. 19C), which motivated us to examine malignant program spatial associations at a more granular level. To this end, Applicant computed the fold-change of each deconvolved immune cell type proportion between the highest quartile-scoring and the lowest quartile-scoring ROIs for each malignant and CAF program (FIG. 20A; Methods). ROIs with high neuronal-like and neuroendocrine-like program scores were significantly enriched with CD8⁺ T cells and depleted of cDC1s; the former was also enriched in cDC2s (FIG. 20A). In contrast, high-scoring squamoid, basaloid, or mesenchymal ROIs were depleted of CD8⁺ T cells; the squamoid program associated with B cells and the basaloid and mesenchymal programs associated with all DC subsets except cDC2s (FIG. 20A). ROIs with high classical program scores were enriched with CD4⁺ T cells (FIG. 20A). Similarly, the neurotropic CAF program was positively associated with CD8⁺, CD4⁺, and regulatory T cells and negatively with activated DCs and cDC1s; the myofibroblastic progenitor and adhesive programs were only positively associated with macrophages and cDC2s, respectively; and the immunomodulatory program was positively associated with activated DCs, cDC1s, plasmacytoid DCs, and plasma cells and negatively with CD4⁺ T cells and macrophages (FIG. 20A). Thus, integrating the snRNA-seq programs and cell signatures with high-resolution spatial transcriptomic data provided a detailed classification of each malignant lineage program and its associated state programs, CAF phenotypes, immune milieu, and clinical outcomes (FIG. 20B).

### Spatially-Associated Receptor-Ligand Pairs Related to Treatment Status

Finally, Applicant identified receptor-ligand (RL) pairs co-expressed across ROIs in either CRT or untreated samples; these may facilitate interactions between malignant, CAF, and immune compartments (FIG. 20C, Methods). Among the correlated pairs were ones with known roles in PDAC¹²⁴⁻¹²⁷, including CXCL12-CXCR4, IGF2-IGF1R, GDNF-RET, and GDNF-GFRA1 (FIG. 20C). Interestingly, the expression of CXCL12 and CXCR4 in epithelial and immune AOIs, respectively, was the most differentially correlated interaction in CRT (positively correlated) vs. untreated (negatively correlated) ROIs (FIG. 20C). CXCL12 expression on tumor cells has been shown to suppress the migration of immune cells such as cytotoxic lymphocytes, and clinical inhibition of CXCR4 with AMD3100 induced an integrated immune response¹²⁵. The correlated expression of CXCL12-CXCR4 post-treatment may suggest a rationale for modulation of this axis to facilitate anti-tumor immunity during or after CRT. TNFSF15-TNFRSF25 is another epithelial-immune RL pair positively correlated only after CRT (FIG. 20C). TNFRSF25 agonism has costimulatory effects on CD4⁺ and CD8⁺ T-cells, promoting accumulation, expansion, and cytotoxic effector function of CD8⁺ T-cells¹²⁸, consistent with the enrichment of T lymphocytes in the “treatment-enriched” neighborhood (FIG. 19C). In addition to interactions purported to have an effect on lymphocyte recruitment, Applicant also note correlated CXCL3 CXCL5-CXCR2 epithelial-immune interactions in CRT ROIs, which have been associated with neutrophil recruitment at inflammatory sites¹²⁹. Beyond malignant cells, CAFs are also important mediators of the immune microenvironment^{54,78,130,131}. Among CAF-immune interactions in CRT ROIs, Applicant observed well-correlated interactions between CXCL6-CXCR1, CXCL12-CXCR4, and CCL19-CCR7, the latter of which is associated with tertiary lymphoid structures and lymph nodes more broadly¹³². The presence of tertiary lymphoid structures has been associated with clinical benefit in cancer patients¹³³.

Among epithelial-CAF correlated RL pairs, there was a marked increase in the correlation in CRT samples of ILIA-ILIR1 and TNF-TNFRSF21 (epithelial-CAF), as well as IGF2-IGF1R and TNF-TRAF2 (CAF-epithelial) (FIG. 20C). The increase in ILIA-ILIR1 correlation is consistent with the pro-inflammatory effects of cytotoxic therapy¹³⁴, though prior studies have associated IL-1α signaling with a pro-tumorigenic niche¹³⁵, suggesting that disruption of the IL-1α pathway may sensitize malignant cells to CRT. Conversely, there is reduced correlation in CRT samples of epithelial-CAF RL pairs involving CXCR2 in the epithelial compartment (e.g., XCL2-CXCR2, CXCL8-CXCR2, FIG. 20C). Genetic ablation and chemical inhibition of CXCR2 in a KPC model of pancreatic cancer significantly reduces metastatic potential, enhances infiltration of T cells, and extends survival in combination with anti-PD1 therapy;³⁶, attenuated CXCR2 interactions post-CRT may therefore be a potential mechanism through which CRT can potentiate a response to immune checkpoint blockade (ICB).

In addition, there were significantly correlated within-compartment RL pairs (FIG. 39 Epithelial-epithelial correlated RL pairs included CCL14-CCR5, NPY-DPP4, and IL2-IL2RG in untreated ROIs (FIG. 39). NPY is cleaved by the peptidase DPP4 followed by preferentially signaling through the NPY2R receptor¹³⁷, which is upregulated in PanINs and invasive adenocarcinoma relative to normal pancreas in mice and humans¹³⁸. In CRT ROIs, correlated RL pairs involve ERBB2 (e.g., HRG-ERBB2, HBEGF-ERBB2, LICAM-ERBB2, NRG1-ERBB2, FIG. 39), consistent with the role of HER2 signaling in resistance to irinotecan/SN-38 and gemcitabine in PDAC cell lines¹³⁹. Although HER2 is not generally associated with direct ligands, interactions with HRG, HBEGF, L1CAM, and NRG1 have been shown to modulate its signaling activity¹⁴⁰⁻¹⁴³. Immune-immune correlated RL pairs included FAS-FASLG (untreated), IL33-ILIR1 (CRT), and IL10-IL10RA (CRT) (FIG. 39). Although IL10 is generally thought of as a potent anti-inflammatory cytokine¹⁴⁴, recent evidence suggests that IL-10-based interactions may metabolically reprogram dysfunctional CD8⁺ T cells to enhance anti-tumor immunity¹⁴⁵.

### Discussion

In this study, Applicant applied single-nucleus RNA-seq to a large cohort of banked frozen primary human pancreatic cancer specimens and demonstrated that this approach was compatible with untreated and heavily pre-treated specimens (FIGS. 17C-17D; FIGS. 21A-21B). Our technique yielded data of higher-quality and better cell type representation than scRNA-seq (FIG. 23)⁵⁶ but also resulted in some immune subset differences compared to in situ analyses, though small numbers of matched samples limits systematic interpretation (FIG. 17E; FIG. 24). With this data, Applicant sought to provide clarity to the cell-type-of-origin of human pancreatic cancer, a topic that has been well-studied in ex vivo and in vivo model systems¹⁴⁶ but has been more challenging to examine in humans. Here, Applicant showed evidence for cells expressing both acinar and ductal markers (putatively ADM) and provide support for a path of transformation from acinar to ADM to ductal to atypical ductal to malignant cells in patient tumors (FIGS. 17F-17G).

Our de novo expression programs captured a refined and expanded cell taxonomy of malignant cells and CAFs in PDAC (FIG. 18A). These include a myofibroblastic-progenitor program that corresponds with previously-defined myofibroblastic signatures but also contains additional elements important in mesoderm development^{54,78}, as well as three other fibroblast programs (immunomodulatory, adhesive, neurotropic) that partition a previously-defined inflammatory CAF annotation^{54,78} and is partially consistent with fibroblast lineage signatures defined in malignant, inflammatory, and fibrotic contexts in the human pancreas, colon, and lung (FIG. 28B)¹¹¹. The lineage programs intrinsic to malignant cells recapitulated a classical program and three distinct squamoid, basaloid, and mesenchymal programs that have generally been thought of as an aggregate “basal-like”/“squamous”/“quasi-mesenchymal” subtype⁷⁻⁹. Applicant also identified neuroendocrine-like and acinar-like programs supporting the existence of the aberrantly-differentiated endocrine exocrine (ADEX) subtype at the malignant cell level (rather than non-malignant tissue contamination^{5,9,10}), and uncovered a novel neuronal-like program that mimics a neuronal differentiation state.

Prior studies have characterized untreated PDAC⁷⁻¹⁰, but there is an unaddressed critical need to dissect the cellular ecosystem of tumors after standard neoadjuvant cytotoxic therapies, which may help guide improvements in both neoadjuvant and adjuvant treatment strategies. While matched pre- and post-treatment specimens would have been optimal, our study was not designed as a correlative biomarker component of an ongoing interventional study. Moreover, cases in our study were often diagnosed with a fine needle aspiration or core needle biopsy at external institutions prior to referral for multidisciplinary management such that the scant material generated by these diagnostic procedures were unavailable to us and would also be limited by a high degree of sampling bias.

Nevertheless, with a large cohort and statistical adjustments to account for the patient/tumor origin of each cell, Applicant found that the novel neuronal-like program was enriched in all post-treatment groups-including the CRT group in situ by DSP (FIGS. 18D, 31, and 37)—and was associated with shortest time to progression (and trending towards worse overall survival) by multivariable Cox regression analysis on bulk RNA-seq data from two independent cohorts (FIGS. 18E and 34). The co-occurrence of several genes involved in the negative regulation of cell death and chemoresistance (e.g., BCL2, PDGFD, SPP1)¹⁴⁷⁻¹⁵⁰ may nominate a potential mechanism of treatment evasion in the neuronal-like program. It remains to be seen whether pro-apoptotic agents, such as the BH3-mimetic venetoclax that blocks BCL2, can inhibit cells expressing this program¹⁵¹. In addition, the program also involves neuronal migration and axonal guidance genes (e.g., CNTN4, RELN, NRP1, DCDC2, SEMA5A, NCAM1, AUTS2)¹¹²⁻¹⁵⁸ that may reflect the adoption of neuronal-like phenotypes to enhance malignant behavior or microenvironmental interactions with nerves, the latter of which is supported by the specific enrichment of previously identified PNI-associated genes in the program (FIG. 29). Tumor-nerve crosstalk has been associated with pro-tumoral phenomenon including perineural invasion, dissemination, post-treatment recurrence, and even metabolic support^{94,159}.

The relative dearth of classical program expression and enrichment in mesenchymal program expression in the setting of FOLFIRINOX is consistent with observations in vivo¹⁴ and ex vivo¹⁶⁰, but the lower expression of the squamoid program in post-treatment residual malignant cells was unexpected, as the squamous phenotype was previously aggregated with the basal-like and quasi-mesenchymal subtypes correlated with poor treatment response and outcomes^{5,7-9}. However, our squamoid program does not overlap significantly with the bulk-defined squamous subtype⁹, which included cell adhesion, stemness, motility, and EMT genes now partitioned into our basaloid and mesenchymal programs. Despite its depletion in post-treatment tumors, the squamoid program was also associated with poor prognosis (FIGS. 18D-18E). This may be consistent with previous reports that the basal-like A phenotype, distinguished by higher expression of squamous differentiation programs, is enriched in metastatic disease, suggesting a greater propensity for dissemination⁷⁶. Note that Applicant did not observe a significant depletion of the classical and squamoid programs in the DSP data (FIGS. 18D and 37), possibly due to selection of tumors with poor treatment response to facilitate recovery of ROIs with adequate cancer cells for downstream analyses.

The post-treatment enrichment of the neuroendocrine-like program is substantiated by a prior study that detailed MYC-regulated ductal to neuroendocrine lineage plasticity as a plausible mechanism for chemotherapy resistance in pancreatic cancer¹⁰². While the neuronal-like program was enriched in both the CRT and CRTL cohorts, the neuroendocrine-like program was only enriched in CRT (FIG. 18D). The cognate receptor for angiotensin II, AGTR1, is highly expressed in some human neuroendocrine neoplasm cell lines that adopt an activated secretory neuroendocrine phenotype upon stimulation with angiotensin II¹⁶¹. Thus, blockade of AGTR1 with losartan may have resulted in selective suppression of the neuroendocrine-like program and offers a potential explanation for the lack of enrichment seen in the CRTL group.

The enrichment of certain malignant cell and CAF programs and depletion of others post-treatment may result from selection of pre-existing phenotypes and/or treatment-induced plasticity. While the absence of matched pre- and post-treatment specimens limits our interpretation, the presence of the mesenchymal, neuronal-like, and neuroendocrine-like phenotypes in untreated specimens, albeit at lower prevalence, and the monotonic increase in the neuronal-like program (and depletion of the classical and squamoid programs) with increasing treatment response all support a model where treatment-mediated selection of pre-existing phenotypes contributes to the features of residual disease (FIGS. 19B, 18D, 31, and 33). However, these enrichment/depletion patterns are also significant when comparing poor treatment response specimens with abundant residual disease to samples from untreated patients, suggesting that phenotypic plasticity may also be playing a role (FIG. 33). Future work comparing matched pre- and post-treatment specimens and functional studies in preclinical models with genetic tracing¹⁶² should provide further insights into the relative contributions of treatment-induced plasticity vs. selection of pre-existing phenotypes.

Combining snRNA-seq with spatial transcriptome profiling identified how the different malignant and stromal programs relate to each other and to immune cell composition in distinct intratumoral microniches (FIGS. 19A-19D). Unsupervised hierarchical clustering of a ROI-based feature correlation matrix revealed three distinct spatial neighborhoods, which were annotated as classical, squamoid-basaloid, and treatment-enriched (FIGS. 19C-19D). In particular, within the substructure of the treatment-enriched neighborhood, the neuronal-like and neuroendocrine-like malignant programs colocalized with the neurotropic CAF programs as well as CD8⁺ T cells. Further investigation into the functional interplay among these cell types/states is warranted to inform potential therapeutic strategies targeting this spatial module that is enriched after neoadjuvant CRT. The spatial associations that were also observed in the orthogonal snRNA-seq feature correlation matrix analysis (FIG. 38) are likely the most robust findings, including the co-occurrence of the neuronal-like malignant program with the neurotropic CAF program and CD8⁺ T cells.

Correlated receptor-ligand interactions, especially those that are differentially correlated between untreated and CRT tumors (FIG. 20C) may functionally underpin these neighborhoods. For example, CXCL12-CXCR4, the most differentially correlated RL pair between epithelial and immune cells is known to have potent immunosuppressive, T cell excluding and pro-tumoral effects in PDAC^{125,163} although Applicant did not observe T cell exclusion in CRT-treated ROIs or snRNA-seq (FIG. 18A and FIG. 26). This may reflect the difference between the primary and metastatic setting^{125,164}, other concomitant modulators, or the time (range 1.7-9.9 weeks) from CRT completion to surgical resection. Our results suggest further investigation of the potential benefit of AMD3100, a small-molecule CXCR4 inhibitor that promotes the accumulation and activation of intratumoral cytotoxic T cells in human PDAC and mouse models^{125,163,164}, for the context of primary disease with neoadjuvant CRT or in the adjuvant setting. Similarly, the correlated RL interactions involving ERBB2 in CRT suggests that HER2 inhibition may have therapeutic efficacy in concert with neoadjuvant CRT (FIG. 20C), despite previous challenges in clinical trials^{165-167,168}.

In conclusion, our study provides a high-resolution molecular framework for understanding the inter- and intra-tumoral diversity of pancreatic cancer; treatment-associated remodeling; spatial organization in discrete neighborhoods with distinct malignant, fibroblast, and immune composition; and clinically-relevant prognostication. These findings can be harnessed to augment precision oncology efforts in pancreatic cancer.

### Methods

### Human Patient Specimens

For inclusion in this study, patients had non-metastatic pancreatic ductal adenocarcinoma and went to surgical resection with or without prior neoadjuvant treatment in the form of chemotherapy and radiotherapy. Most treated patients received several cycles of FOLFIRINOX chemotherapy (5-FU, leucovorin, irinotecan, oxaliplatin) followed by multi-fraction radiotherapy with concurrent capecitabine or 5-FU. Four patients received other forms of chemotherapy such as cisplatin, gemcitabine, or nab-paclitaxel. Seven patients received additional neoadjuvant therapy in the form of losartan, an angiotensin II receptor type 1 antagonist, and/or nivolumab, a PD-1 inhibitor, on two clinical trials (NCT03563248, NCT01821729). The most common radiotherapy regimens included 30 Gy in 10 fractions, 50.4 Gy in 28 fractions (with dose painting up to 58.8 Gy to cover high-risk areas such as tumor-vessel interfaces), and stereotactic body radiotherapy 36 Gy in 6 fractions (with dose painting up to 42 Gy to cover high-risk areas such as tumor-vessel interfaces). Conformal techniques, most commonly volumetric modulated arc therapy, were employed for treatment delivery.

All patients were consented to excess tissue biobank protocol 2003P001289 (principal investigator: CFC; co-investigators: ASL, WLH), which was reviewed and approved by the Massachusetts General Hospital (MGH) Institutional Review Board. Resected primary tumor samples were examined to confirm neoplastic content by a board-certified pathologist (MMK) and then snap frozen and stored at −80° C. for up to 5 years prior to processing. Specimens were screened for an RNA integrity number (RIN; Agilent RNA 6000 Pico Kit, cat. No. 5067-1513) greater than an empirically determined threshold of 6; only specimens with RIN>6 were processed further. In many cases, matched formalin-fixed paraffin-embedded (FFPE) blocks were used for multiplexed ion beam imaging (MIBI, Ionpath) and digital spatial profiling (DSP, Nanostring).

Nucleus Isolation from Frozen Samples

Applicant have recently published a toolbox for snRNA-seq of tumors spanning a broad range of nucleus isolation techniques for various tissue/tumor types⁴⁵, but not PDAC. The following protocol is an adaptation and optimization of this prior work specifically for the unique tissue requirements of pancreatic tumors. A 2× stock of STc buffer in nuclease-free water was prepared with a final concentration of 292 mM NaCl (ThermoFisher Scientific, cat. no. AM9759), 40 mM Tricine (VWR, cat. no. E170-100G), 2 mM CaCl₂ (VWR, cat. no. 97062-820), and 42 mM MgCl₂ (Sigma Aldrich, cat. no. M1028). For each specimen, 2 mL of NSTcPA buffer was prepared by combining 1 mL of 2× STc buffer, 40 μL of 10% Nonidet P40 Substitute (Fisher Scientific, cat. no. AAJ19628AP), 10 μL of 2% bovine serum albumin (New England Biolabs, cat. no. B9000S), 0.3 μL of 1M spermine (Sigma-Aldrich, cat. no. 53256-1G), 1 μL of 1M spermidine (Sigma-Aldrich, cat. no. 52626-1G), and 948.7 μL of nuclease-free water. For each specimen, 3 mL of 1× working STc buffer was made by diluting 2× STc 1:1 in nuclease-free water.

NSTcPA buffer (1 mL) was pipetted into one well of a 6-well plate (Stem Cell Technologies, cat. no. 38015) on ice. The frozen tumor specimen was removed from −80° C. and placed in a petri dish on dry ice. Using a clean razor blade, the desired regions of the tissue were cut on dry ice so the specimen remained frozen. The amount of each tumor processed for snRNA-seq varied but was typically 20-50 mg; fragments from several regions of the tumor were processed together to reduce spatial sampling bias. The remainder of the specimen was returned to −80° C. for subsequent use. The selected tissue was transferred into the NSTcPA buffer and manually minced with fine straight tungsten carbide scissors (Fine Science Tools, cat. no. 14568-12) for 8 minutes. The homogenized tissue solution was then filtered through a 40 μm Falcon cell filter (Thermo Fisher Scientific, cat. no. 08-771-1) into a 50 mL conical tube. An additional 1 mL of NSTcPA buffer was used to rinse the well and filter. The total volume was brought up to 5 mL with 3 mL of 1× STc buffer and transferred into a 15 mL conical tube. The sample was spun for 5 min at 500 xg, 4° C. and the supernatant was removed. The pellet was resuspended in 100-200 μL 1× STc and then filtered through a 35 μm Falcon cell strainer (Corning, cat. no. 352235). Nuclei were quantified using a C-chip disposable hemocytometer (VWR, cat. no. 82030-468) and diluted in 1× STc as necessary to achieve a final concentration of 300-2,000 nuclei/μL.

Single-Nucleus RNA-Seq (snRNA-Seq)

Approximately 8,000-10,000 nuclei per sample were loaded into each channel of a Chromium single-cell 3′ chip (V2 or V3, 10× Genomics) according to the manufacturer's instructions. Single nuclei were partitioned into droplets with gel beads in the Chromium Controller to form emulsions, after which nucleus lysis, barcoded reverse transcription of mRNA, cDNA amplification, enzymatic fragmentation, and 5′ adaptor and sample index attachment were performed according to manufacturer's instructions. Up to four sample libraries were sequenced on the HiSeq X Version 2.5 (Illumina) with the following paired end read configuration: read 1, 26-28 nt; read 2, 96-98 nt; index read, 8 nt.

snRNA-Seq Data Pre-Processing

BCL files were converted to FASTQ using bcl2fastq2-v2.20. CellRanger v3.0.2 was used to demultiplex the FASTQ reads, align them to the hg38 human transcriptome (pre-mRNA) reference and extract the UMI and nuclei barcodes. The output of this pipeline is a digital gene expression (DGE) matrix for each sample, which has quantified for each nucleus barcode the number of UMIs that aligned to each gene.

Applicant filtered low-quality nuclei profiles by baseline quality control measures including number of reads captured and ambient RNA detection. First, Applicant used CellBender remove-background¹⁶⁹ to remove ambient RNA, enhancing cell distinction and marker specificity. CellBender remove-background was run (on Terra) to remove ambient RNA and other technical artifacts from the count matrices. The workflow is available publicly as cellbender/remove-background (snapshot 11) and documented on the CellBender github repository as v0.2.0: https://github.com/broadinstitute/CellBender. This latest version of CellBender remove-background cleans up count matrices using a principled model of noise generation in scRNA-Seq. The parameters “expected-cells” and “total-droplets-included” were chosen for each dataset based on the total UMI per cell vs. cell barcode curve in accordance with CellBender documentation. Other inputs were left at their default values. The false positive rate parameter “fpr” was set to 0.01, 0.05, and 0.1. For downstream analyses Applicant used the ‘FPR_0.01_filtered.h5’ file. Following this step, Applicant filtered out nuclei with over 10,000 UMI counts. To account for differences in sequencing depth across nuclei, UMI counts were normalized by the total number of UMIs per nucleus and converted to transcripts-per-10,000 (TP10K) as the final expression unit.

### Dimensionality Reduction, Clustering and Annotation

Following these quality control steps, treatment-naive and neoadjuvant-treated specimens were aggregated into a single joint dataset. The log₂(TP10K+1) expression matrix was constructed for the following downstream analyses. Applicant identified the top 2,000 highly variable genes across the entire dataset using the Scanpy¹⁷⁰ highly variable genes function with the sample id as input for the batch. Applicant then performed a Principal Component Analysis (PCA) over the top 2,000 highly variable genes and identified the top 40 principle components (PCs) beyond which negligible additional variance was explained in the data (the analysis was performed with 30, 40, and 50 PCs and robust to this choice). Subsequently, Applicant performed batch correction using Harmony-Pytorch¹⁷¹ and built a k-nearest neighbors graph of nuclei profiles (k=10) based on the top 40 batch corrected components and performed community detection on this neighborhood graph using the Leiden graph clustering method¹⁷² with resolution set to 1 to identify distinct cell population clusters. Individual nuclei profiles were visualized using the uniform manifold approximation and projection (UMAP)¹⁷³. Distinct cell populations identified from the previous steps were annotated using known cell type-specific gene expression signatureS^{42,47-50}. Representative marker genes included but were not limited to: ductal (CFTR), malignant epithelial (KRT7, KRT17, KRT19, EPCAM, CEACAM6), acinar (CPB1, PRSS3, AMY1A), acinar-REG+(REG3A, REG3G, REG1B), cancer-associated fibroblast (COLIA1, FN1, PDPN, DCN, VIM, FAP, ACTA2), vascular smooth muscle (MYH11 , MYOCD), pericyte (PDGFRB, DLK1, ACTA1 , RGS5, CSPG4, MCAM), endothelial (PECAM1, VWF), vascular endothelial (ESAM, FLT1, EPAS1 ), lymphatic endothelial (FLT4, SEMA3A, SEMA3D), adipocyte (PLIN1 , LPL), alpha (GCG), beta (INS, IAPP), delta (SST), gamma (PPY), epsilon (GHRL), neuroendocrine (SYP, CHGA), intrapancreatic neurons (TH, CHAT, ENO2, NSE), Schwann (SOX10, S100B), pan-immune (PTPRC), antigen-presenting cell (CD74), macrophage (CD68, CD163, MRC1, CD80, CD86, TGFBI, CSF1), monocyte (TLR2, ITGB2, ITGAM, CTSD, CTSA, NLRP3, CLEC7A, BST1, STAB1 , IRAK3), eosinophil (MBP, EDN, EPO, CCR3), cDC1 (XCR1, CST3, CLEC9A, LGALS2), cDC2 (CD1A, CD207, CDE, FCER1A, NDRG2), activated DC (FSCN1, LAMP3, CCL19, CCR7), plasmacytoid DC (GZMB, IRF7, LILRA4, TCF4, CXCR3, IRF4), T cell (CD4, CD8A, CD8B, CD3D, THEMIS, CD96, IKZF1, GZMA, FOXP3), B cell (BANK1 , CD19), NK cell (KLRD1 , KIR2DL3, IL18R1, KIR2DL1, KIR3DL2), plasma (SDC1 , IGLC2), mast (CPA3, KIT), neutrophil (CSF3R, CXCL8). The Adjust Mutual Information (AMI) score measures the consistency between two clustering assignments over all cells. Applicant used the AMI to quantify the similarity in single cell assignments between the Leiden clustering labels and patient ID labels. The AMI was computed using the adjusted_mutual_info_score function in the Python sklearn package.

### Quantifying Statistically Significant Changes in Composition Between Cell Populations

To compute the significance of changes in the cellular composition between untreated and treated (CRT and CRTL) samples, Applicant used multiple statistical tests that each capture different types of information: (1) Dirichlet-multinomial regression, and (2) Mann-Whitney test. To account for dependencies among cell proportions (an increase in the proportion of one cell subset will necessarily lead to a decrease in the proportions of the other cell subsets), Applicant used a Dirichlet-multinomial regression. This statistical test and its inclusion probabilities (pi) were calculated using the “scCODA” Python package¹⁷⁴. Applicant also performed a non-parametric Mann-Whitney U test on the proportions of each cell subset in untreated versus treated (CRT and CRTL) samples. These same statistical approaches were applied to quantify the differences in cells/nuclei captured by the snRNA-seq approach and a previously published scRNA-seq method⁵⁶.

Inferring Copy Number Aberrations from Single Nucleus Profiles

A Python implementation of InferCNV v3.9 based on the InferCNV R implementation as provided at https://github.com/broadinstitute/inferCNV (inferCNV of the Trinity CTAT Project) was run jointly on all treated and untreated single nuclei profiles. To avoid circularity, Applicant used a set of high confidence non-neoplastic cells as the reference that was derived from two non-malignant pancreas snRNA-seq samples. Applicant used the default parameters for InferCNV including a 100-gene window in sub-clustering mode and a hidden Markov model to predict the copy number aberration (CNA) count and construct a CNA score for each nucleus based on the predicted CNAs in each cell. Annotated epithelial cells were subject to Leiden sub-clustering and those with an average CNA score greater than 0.01 were labeled as malignant epithelial cells.

### Partition-Based Graph Abstraction

The pseudotemporal orderings/trajectories of annotated epithelial cell types was estimated using the diffusion map and partition-based graph abstraction (PAGA v1.2) method⁶⁴. The diffusion map was computed with 15 components and the cell neighborhood map utilized a local neighborhood of 15.

### Multiplexed Ion Beam Imaging (MIBI)

Formalin-fixed paraffin-embedded pancreatic tissue sections were cut onto gold MIBI slides (IONpath, cat. no. 567001) and stained at IONpath (Menlo Park, CA) with the internal Epithelial i-Onc isotopically-labelled antibody panel (IONpath): dsDNA_89 [3519 DNA] (1:100), β-tubulin_166 [D3U1W] (3:200), CD163_142 [EPR14643-36] (3:1600), CD4_143 [EPR6855] (1:100), CD11c_144 [EP1347Y] (1:100), LAG3_147 [17B4] (1:250), PD-1_148 [D4W2J] (1:100), PD-L1_149 [E1L3N] (1:100), Granzyme B_150 [D6E9W](1:400), CD56_151 [MRQ-42] (1:1000), CD31_152 [EP3095] (1:1000), K1-67_153 [D2H10](1:250), CD11b_155 [D6X1N] (1:500), CD68_156 [D4B9C] (1:100), CD8_158 [C8/144B](1:100), CD3_159 [D7A6E] (1:100), CD45RO_161 [UCHL1] (1:100), Vimentin_163 [D21H3] (1:100), Keratin_165 [AE1/AE3] (1:100), CD20_167 [L26] (1:400), Podoplanin_170 [D2-40] (1:100), IDO1_171 [EPR20374] (1:100), HLA-DR_172 [EPR3692] (1:100), DC-SIGN_173 [DCN46] (1:250), CD45_175 [2B11 & PD7/26] (3:200), HLA class 1 A, B, and C_176 [EMR8⁻⁵] (1:100), Na/K-ATPase_176 [D4Y7E] (1:100).

Quantitative imaging was performed using a beta unit MIBIscope (IONpath) equipped with a duoplasmatron ion source. This instrument sputters samples with O₂⁺ primary ions line-by-line, while detecting secondary ions with a time-of-flight mass spectrometer tuned to 1-200 m/z+ and mass resolution of 1000 m/Δm, operating at a 100 KHz repetition rate. The primary ion beam was aligned daily to minimize imaging astigmatism and ensure consistent secondary ion detection levels using a built-in molybdenum calibration sample. In addition to the secondary ion detector, the MIBIscope is equipped with a secondary electron detector which enables sample identification and navigation prior to imaging.

For data collection, three fields of view were acquired for each sample by matching the secondary electron morphological signal to annotated locations on sequential H&E stained slides. The experimental parameters used in acquiring all imaging runs were as follows: pixel dwell time (12 ms), image size (500 μm² at 1024×1024 pixels), primary ion current (5 nA O₂⁺), aperture (300 m), stage bias (+67 V).

### MIBI Image Processing Segmentation and Quantification

Mass spectrometer run files were converted to multichannel tiff images using MIB.io software (IONpath). Mass channels were filtered individually to remove gold-ion background and spatially uncorrelated noise. HLA Class 1 and Na/K-ATPase signals were combined into a single membrane marker. These image files (tiff) were used as a starting point for single cell segmentation, quantification and interactive analysis using histoCAT (v1.76)¹⁷⁵. Applicant followed a similar approach for segmentation as proposed for Imaging Mass Cytometry data¹⁷⁵⁻¹⁷⁷. Briefly, Applicant used Ilastik¹⁷⁸ to manually train three classes (nuclei, cytoplasm and background) to improve subsequent watershed segmentation using CellProfiler¹⁷⁹. Finally, the tiff images and masks were combined for histoCAT loading with a script optimized for MIBI image processing (code, classifiers and configuration files are available at https://github.com/DenisSch/MIBI).

The immune cells were further stratified into cell subsets by incorporating the full set of protein markers available along with the untreated and treated snRNA-seq data. First, Applicant used the gimVI variational autoencoder to train a model¹⁸⁰ taking both spatial MIBI and snRNA-seq data modalities as well as the correspondence between genes and antibody markers as input and encoding both the MIBI and snRNA-seq datasets into a joint latent space. The gimVI model was trained for 10 epochs. The latent space representation of the snRNA-seq data was then extracted from this model and used as the features to build a random forest model for cell type classification. Subsequently, the latent space representation extracted for each MIBI image was then evaluated using our trained model to generate a predicted cell type for each segmented immune cell in the spatial data.

### Differential Gene Expression Analysis

For each annotated cell type detected in both untreated and treated tumors, a differential gene expression analysis using a mixed effects Poisson model was performed between cells in the two populations to identify upregulated and downregulated genes. Applicant considered untreated, all treated, CRT, and CRTL treatment categories in this analysis. Applicant constructed a mixed effects model with the sample ID as a random effect; treatment status, two principal components and sex were fixed effect covariates; and finally, the log total UMIs as an offset. The mixed effects model was implemented using the glmer R package¹⁸¹.

### Scoring Gene Signatures for Each Nucleus Profile

A signature score for each nucleus profile was computed as the mean log₂(TP10K+1) expression across all genes in the gene signature. Subsequently, to identify statistically significant gene expression patterns, Applicant computed the mean log₂(TP10K+1) expression across a background set of 50 genes randomly selected with matching expression levels to those of the genes in the signature iterated 25 times. The gene signature score was defined to be the excess in expression found across the genes in the signature compared to the background set.

### Consensus Non-Negative Matrix Factorization

Applicant formulated the task of dissecting gene expression programs as a matrix factorization problem where the input gene expression matrix is decomposed into two matrices. The solution to this formulation can be identified by solving the following minimization problem:

$$arg min \{ \frac{1}{2} {‖ {X}_{n , m} - {W}_{n , p} \times {H}_{p , m} ‖}_{F}^{2} + ( 1 - α ) \frac{1}{2} ‖ {W}_{n , p} ‖ + \frac{1}{2} ( 1 - α ) ‖ {H}_{n , p} ‖ + α {‖ vec ( {W}_{n , p )} ‖}_{1} + α {‖ vec ( {H}_{n , p} ) ‖}_{1} \}$$

Applicant utilized the non-negative matrix factorization implemented in sklearn to derive the malignant and CAF expression programs across both untreated and treated samples. Because the result of NMF optimization can vary between runs based on random seeding, Applicant repeated NMF 50 times per cell type category and computed a set of consensus programs by aggregating results from all 50 runs and computed a stability and reconstruction error. This consensus NMF was performed by making custom updates to the cNMF python package⁷⁹. To determine the optimal number of programs (p) for each cell type and condition, Applicant struck a balance between maximizing stability and minimizing error of the cNMF solution, while ensuring that the resulting programs were as biologically coherent and parsimonious as possible. Each program was annotated utilizing a combination of GSEA¹⁸² and comparison to bulk expression signatures.

### Measuring Similarity Between Gene Expression Programs

To measure the similarity between cNMF-derived gene expression programs and pre-existing bulk derived gene sets representing PDAC subtypes or differentially-expressed genes associated with perineural invasion, Applicant performed the hypergeometric test and Kolmogorov-Smirnov test, respectively, to quantify the overlap between the two gene sets. This test enables us to determine enrichment or depletion of gene expression programs in a pre-defined gene set. To measure the similarity among the cNMF-derived gene expression programs Applicant computed the correlation of the cell by program vector for each program to identify which programs were found to be co-occurring across the same cells. Finally, Applicant computed the patient-level statistical comparisons of program compositional changes by treatment type and response. This were performed by computing the average program weight over all cells for each patient and testing for changes to the program abundance using statistical tests as described in the prior section on quantifying statistically significant changes in cell composition between cell populations.

### Survival Analysis of Bulk RNA-Seq Data

Bulk RNA-seq data from two previously published resected primary PDAC cohorts with overall survival annotated were obtained (The Cancer Genome Atlas, n=139; PanCuRx, n=168)^{10,14,76}. Patients with metastases or those that received neoadjuvant therapy were excluded from this analysis, yielding a total of 269 patients for further analysis. Gene expression levels from RNA-seq data was estimated using RSEM¹⁸³.

To score malignant and fibroblast cNMF programs in each tumor, Applicant summed the expression of the top 200 genes for each program and z-score normalized the expression scores within the TCGA and PanCuRx cohorts independently to account for batch effects. Age, sex, grade and stage were available for all patients. There were 154 progression events and 167 deaths. Since there were four clinicopathologic covariates and 18 gene expression programs across the malignant and CAF compartments, Applicant sought to consolidate some of the GEPs into aggregate programs to avoid overfitting a Cox proportional-hazards regression model. Towards this end, Applicant noted that among the malignant cell state programs, the TNF-NFκB, adhesive, interferon, and ribosomal programs featured 7-17% secreted proteins while the cycling (S), cycling (G2/M), and MYC programs exhibited 1% or fewer secreted proteins¹⁸⁴. This allowed us to aggregate the seven malignant cell state programs into two binary categories: cycling (cycling (S), cycling (G2/M), MYC) and secretory (TNF-NFκB, interferon, adhesive, ribosomal), yielding a total of 17 covariates in the Cox proportional-hazards regression model. Multivariable Cox regression analyses was performed for time to progression (TTP) and overall survival (OS) (Stata/SE 15.1).

### Digital Spatial Profiling—Experimental

Applicant followed published experimental methods¹²′ (Nanostring) with modifications as noted below. Briefly, serially sectioned formalin-fixed paraffin-embedded (FFPE) sections (5 μm) of 21 specimens were prepared by the MGH Histopathology Core on the IRB-approved protocol (2003P001289) to generate consecutive sections that were processed for H&E and WTA, respectively. For WTA, slides were baked at 60° C. for one hour, deparaffinized with CitriSolv (DECON), rehydrated, antigen-retrieved in 1× Tris-EDTA/pH 9 in a steamer for 15 min at 100° C., proteinase K (ThermoFisher Scientific, AM2548) digested at 0.1 ng/mL for 15 min at 37° C., post-fixed in neutral-buffered formalin for 10 min, hybridized to UV-photocleavable barcode-conjugated RNA in situ hybridization probe set (WTA with 18,269 targets) overnight at 37° C., washed to remove off-target probes, and then counterstained with morphology markers for 2 hours.

The morphology markers consisted of: 1:10 SYTO13 (ThermoFisher Scientific, cat. no. 57575), 1:20 anti-panCK-Alexa Fluor 532 (clone AE-1/AE-3; Novus Biologicals, cat. no. NBP2-33200AF532), 1:100 anti-CD45-Alexa Fluor 594 (clone D9M8I; Cell Signaling Technology, cat. no. 13917S), and 1:100 anti-aSMA-Alexa Fluor 647 (clone 1A4; Novus Biologicals, cat. no. IC1420R). The anti-panCK and anti-aSMA antibodies were acquired pre-conjugated whereas the anti-CD45 antibody was conjugated using the Alexa Fluor 594 Antibody Labeling Kit (Invitrogen, A20185). These four morphology markers allowed delineation of the nuclear, epithelial, immune, and fibroblast compartments. Immunofluorescence images, region of interest (ROI) selection, segmentation into marker-specific areas of interest (AOI), and spatially-indexed barcode cleavage and collection were performed on a GeoMx Digital Spatial Profiling instrument (NanoString). Typical exposure times were 50 ms for SYTO13, 300 ms for anti-panCK-Alexa Fluor 532, 400-450 ms for anti-CD45-Alexa Fluor 594, and 50 ms for anti-aSMA-Alexa Fluor 647. Approximately 8-14 ROIs and 20-36 AOIs were collected per specimen. Library preparation was performed according to the manufacturer's instructions and involved PCR amplification to add Illumina adapter sequences and unique dual sample indices. A minimum sequencing depth of 150-200 reads per square micron of illumination area was achieved by sequencing all WTA AOIs on a NovaSeq S2 (100 cycles, read 1:27 nt, read 2:27 nt, index 1:8 nt, index 2:8 nt).

### Digital Spatial Profiling Data Preprocessing

FASTQ files for DSP were aggregated into count matrices as described previously¹²¹. Briefly, deduplicated sequencing counts were calculated based on UMI and molecular target tag sequences. Single probe genes were reported as the deduplicated count value. The limit of quantitation (LOQ) was estimated as the geometric mean of the negative control probes plus 2 geometric standard deviations of the negative control probes. Targets were removed that consistently fell below the LOQ, and the datasets were normalized using upper quartile (Q3) normalization. Normalized expression was detrended to model cell-type specific expression by calculating an adjustment factor:

A_{s1,g,r}=E_{s1,g,r}*(E_{s1,g,r}−max(E_{s2,g,r},E_{S3,g,r}))

Where adjustment factors, A, are calculated for the expression, E, of a gene, g, within a given ROI, r, by comparing a given segment, S1, to the max expression observed in other segments, S2 and S3. The original expression was then detrended by calculating:

$${D}_{S 1 , g , r} = {E}_{S 1 , g , r} - ( \frac{2 * {E}_{S 1 , g , r}}{max ( {E}_{S 1 , r} )} ) * {log}_{2} ( max ( {A}_{S 1 , g , r} , 1 ) )$$

This resulted in detrended expression, D, reflecting the original expression minus positive adjustment factors scaled based on the relative expression of the target to all other targets in the segment.

### Program Scoring and Program Correlation Analysis

Statistical analysis was performed using R. Programs were scored for each DSP sample within each region of interest using single-sample gene set enrichment analysis (ssGSEA)¹⁸⁵, which were transformed using the z-score. Intra-patient program expression variation was calculated as mean interquartile range (IQR) of a program across all ROIs within a tumor, while inter-patient variation was calculated as the IQR of the mean program score for each tumor. Unsupervised hierarchical clustering was performed on all features (malignant programs, CAF programs, deconvolved immune cell type proportions, compartment areas within ROI) using the Pearson correlation distance and average linkage. Cell deconvolution analysis was performed using the SpatialDecon package (https://github.com/Nanostring-Biostats/SpatialDecon/). Analysis of expression or program scores used linear mixed effects models¹⁸⁶ to control for multiple sampling within a slide, using Satterthwaite's approximation¹⁸⁷ for degrees of freedom for p-value calculation. Correlation coefficients were calculated using the Spearman rank correlation.

### Receptor Ligand (RL) Correlation Across ROIs

Known receptor-ligand pairs were obtained from CellPhoneDB with potential receptor-ligand pairs quantified using the Spearman rank correlation between paired segments within the same ROI across all ROIs with said pairs. Interactions were calculated for non-self (juxtacrine) and self (autocrine) occurring within the same segment. Receptor-ligand interactions were calculated separately for untreated and CRT specimens to determine interactions that are differential between conditions. All analyses were two-sided and used a significant level of p-value ≤0.05 and were adjusted for multiple testing where appropriate using the false discovery rate¹⁸⁸.

### Data Availability

Raw data will be available in the controlled access repository Data Use Oversight System (DUOS) at the Broad Institute: https://duos.broadinstitute.org/under its Data Access Committee. Processed annotated datasets will be provided in the Single Cell Portal upon publication.

### Code Availability

All code will be available upon publication in Github at https://github.com/karthikj89/humanpdac.

### References for Example 2

1. Mizrahi, J. D., Surana, R., Valle, J. W. & Schroff, R. T. Pancreatic cancer. Lancet 395, 2008-2020 (2020).
2. Siegel, R. L., Miller, K. D., Fuchs, H. E. & Jemal, A. Cancer Statistics, 2021. CA. Cancer J. Clin. 71, 7-33 (2021).
3. Versteijne, E. et al. Preoperative Chemoradiotherapy Versus Immediate Surgery for Resectable and Borderline Resectable Pancreatic Cancer: Results of the Dutch Randomized Phase III PREOPANC Trial. J. Clin. Oncol. 38, 1763-1773 (2020).
4. Murphy, J. E. et al. Total Neoadjuvant Therapy with FOLFIRINOX in Combination with Losartan Followed by Chemoradiotherapy for Locally Advanced Pancreatic Cancer: A Phase 2 Clinical Trial. JAMA Oncol. 5, 1020-1027 (2019).
5. Collisson, E. A., Bailey, P., Chang, D. K. & Biankin, A. V. Molecular subtypes of pancreatic cancer. Nat. Rev. Gastroenterol. Hepatol. 16, 207-220 (2019).
6. Guo, J. A. et al. Refining the Molecular Framework for Pancreatic Cancer with Single-cell and Spatial Technologies. Clin. Cancer Res. 27, 3825-3833 (2021).
7. Collisson, E. A. et al. Subtypes of pancreatic ductal adenocarcinoma and their differing responses to therapy. Nat. Med. 17, 500⁻⁵⁰³ (2011).
8. Moffitt, R. A. et al. Virtual microdissection identifies distinct tumor- and stroma-specific subtypes of pancreatic ductal adenocarcinoma. Nat. Genet. 47, 1168-1178 (2015).
9. Bailey, P. et al. Genomic analyses identify molecular subtypes of pancreatic cancer. Nature 531, 47-52 (2016).
10. Raphael, B. J. et al. Integrated Genomic Characterization of Pancreatic Ductal Adenocarcinoma. Cancer Cell 32, 185-203 (2017).
11. Sivakumar, S., de Santiago, I., Chlon, L. & Markowetz, F. Master Regulators of Oncogenic KRAS Response in Pancreatic Cancer: An Integrative Network Biology Analysis. PLoS Med. 14, (2017).
12. de Santiago, I. et al. Immuno-phenotypes of Pancreatic Ductal Adenocarcinoma: Metaanalysis of transcriptional subtypes. bioRxiv (2017) doi:10.1101/198903.
13. Le Large, T. Y. S. et al. Key biological processes driving metastatic spread of pancreatic cancer as identified by multi-omics studies. Semin. Cancer Biol. 44, 153-169 (2017).
14. Aung, K. L. et al. Genomics-driven precision medicine for advanced pancreatic cancer: Early results from the COMPASS trial. Clin. Cancer Res. 24, 1344-1354 (2018).
15. Kroemer, G., Galluzzi, L., Kepp, O. & Zitvogel, L. Immunogenic cell death in cancer therapy. Annu. Rev. Immunol. 31, 51-72 (2013).
16. Coffelt, S. B. & de Visser, K. E. Immune-mediated mechanisms influencing the efficacy of anticancer therapies. Trends Immunol. 36, 198-216 (2015).
17. Hosein, A. N., Brekken, R. A. & Maitra, A. Pancreatic cancer stroma: an update on therapeutic targeting strategies. Nat. Rev. Gastroenterol. Hepatol. 17, 487-505 (2020).
18. Zitvogel, L., Apetoh, L., Ghiringhelli, F. & Kroemer, G. Immunological aspects of cancer chemotherapy. Nat. Rev. Immunol. 8, 59-73 (2008).
19. Burnette, B. C. et al. The efficacy of radiotherapy relies upon induction of type I interferon-dependent innate and adaptive immunity. Cancer Res. 71, 2488-2496 (2011).
20. Gupta, A. et al. Radiotherapy Promotes Tumor-Specific Effector CD8+ T Cells via Dendritic Cell Activation. J. Immunol. 189, 558-566 (2012).
21. Ma, Y. et al. Anticancer chemotherapy-induced intratumoral recruitment and differentiation of antigen-presenting cells. Immunity 38, 729-741 (2013).
22. Zitvogel, L., Galluzzi, L., Kepp, O., Smyth, M. J. & Kroemer, G. Type I interferons in anticancer immunity. Nat. Rev. Immunol. 15, 405-414 (2015).
23. Deng, L. et al. STING-dependent cytosolic DNA sensing promotes radiation-induced type I interferon-dependent antitumor immunity in immunogenic tumors. Immunity 41, 843-852 (2014).
24. Sahai, E. et al. A framework for advancing our understanding of cancer-associated fibroblasts. Nat. Rev. Cancer 20, 174-186 (2020).
25. Hutton, C. et al. Single-cell analysis defines a pancreatic fibroblast lineage that supports anti-tumor immunity. Cancer Cell 1, 1097-1112 (2020).
26. Chauhan, V. P. et al. Angiotensin inhibition enhances drug delivery and potentiates chemotherapy by decompressing tumour blood vessels. Nat. Commun. 4, 2516 (2013).
27. Barker, H. E., Paget, J. T. E., Khan, A. A. & Harrington, K. J. The tumour microenvironment after radiotherapy: Mechanisms of resistance and recurrence. Nat. Rev. Cancer 15, 409-25 (2015).
28. Liu, H. et al. Use of angiotensin system inhibitors is associated with immune activation and longer survival in nonmetastatic pancreatic ductal adenocarcinoma. Clin. Cancer Res. 23, (2017).
29. Sorensen, B. S. & Horsman, M. R. Tumor Hypoxia: Impact on Radiation Therapy and Molecular Pathways. Front. Oncol. 10, 562 (2020).
30. Patel, A. P. et al. Single-cell RNA-seq highlights intratumoral heterogeneity in primary glioblastoma. Science (80-.). 344, 1396-1401 (2014).
31. Tirosh, I. et al. Single-cell RNA-seq supports a developmental hierarchy in human oligodendroglioma. Nature 539, 309-313 (2016).
32. Venteicher, A. S. et al. Decoupling genetics, lineages, and microenvironment in IDH-mutant gliomas by single-cell RNA-seq. Science (80-.). 355, (2017).
33. Puram, S. V et al. Single-Cell Transcriptomic Analysis of Primary and Metastatic Tumor Ecosystems in Head and Neck Cancer. Cell 171, 1611-1624 (2017).
34. Steele, N. G. et al. Multimodal Mapping of the Tumor and Peripheral Blood Immune Landscape in Human Pancreatic Cancer. Nat. Cancer 1, 1097-1112 (2020).
35. Azevedo-Pouly, A. C. P., Elgamal, O. A. & Schmittgen, T. D. RNA isolation from mouse pancreas: A ribonuclease-rich tissue. J. Vis. Exp. e51779 (2014) doi:10.3791/51779.
36. Augereau, C., Lemaigre, F. P. & Jacquemin, P. Extraction of high-quality RNA from pancreatic tissues for gene expression studies. Anal. Biochem. 500, 60-62 (2016).
37. Jun, E. et al. Method Optimization for Extracting High-Quality RNA From the Human Pancreas Tissue. Transl. Oncol. 11, 800-807 (2018).
38. Von Ahrens, D., Bhagat, T. D., Nagrath, D., Maitra, A. & Verma, A. The role of stromal cancer-associated fibroblasts in pancreatic cancer. Journal of Hematology and Oncology vol. 10 (2017).
39. Habib, N. et al. Div-Seq: Single-nucleus RNA-Seq reveals dynamics of rare adult newborn neurons. Science (80-.). 353, 925-928 (2016).
40. Habib, N. et al. Massively parallel single-nucleus RNA-seq with DroNc-seq. Nat. Methods 14, 955-958 (2017).
41. Drokhlyansky, E. et al. The Human and Mouse Enteric Nervous System at Single-Cell Resolution. Cell 182, 1606-1622.e23 (2020).
42. Tosti, L. et al. Single-Nucleus and In Situ RNA-Sequencing Reveal Cell Topographies in the Human Pancreas. Gastroenterology 160, 1330-1344 (2021).
43. Barthelson, R. A., Lambert, G. M., Vanier, C., Lynch, R. M. & Galbraith, D. W. Comparison of the contributions of the nuclear and cytoplasmic compartments to global gene expression in human cells. BMC Genomics 8, 340 (2007).
44. Abdelmoez, M. N. et al. SINC-seq: Correlation of transient gene expressions between nucleus and cytoplasm reflects single-cell physiology. Genome Biol. 19, (2018).
45. Slyper, M. et al. A single-cell and single-nucleus RNA-Seq toolbox for fresh and frozen human tumors. Nat. Med. 26, 792-802 (2020).
46. Han, J., DePinho, R. A. & Maitra, A. Single-cell RNA sequencing in pancreatic cancer. Nat. Rev. Gastroenterol. Hepatol. 18, 451-452 (2021).
47. Gentles, A. J. et al. The prognostic landscape of genes and infiltrating immune cells across human cancers. Nat. Med. 21, 938-945 (2015).
48. Muraro, M. J. et al. A Single-Cell Transcriptome Atlas of the Human Pancreas. Cell Syst. 3, 385-394 (2016).
49. Zilionis, R. et al. Single-Cell Transcriptomics of Human and Mouse Lung Cancers Reveals Conserved Myeloid Populations across Individuals and Species. Immunity 50, 1317-1334 (2019).
50. Schupp, J. C. et al. Integrated Single Cell Atlas of Endothelial Cells of the Human Lung. Circulation 144, 286-302 (2021).
51. Angelo, M. et al. Multiplexed ion beam imaging of human breast tumors. Nat. Med 20, 436-442 (2014).
52. Keren, L. et al. A Structured Tumor-Immune Microenvironment in Triple Negative Breast Cancer Revealed by Multiplexed Ion Beam Imaging. Cell 174, 1373-1387.e19 (2018).
53. Bernard, V. et al. Single-cell transcriptomics of pancreatic cancer precursors demonstrates epithelial and microenvironmental heterogeneity as an early event in neoplastic progression. Clin. Cancer Res. 25, 2194-2205 (2019).
54. Elyada, E. et al. Cross-species single-cell analysis of pancreatic ductal adenocarcinoma reveals antigen-presenting cancer-associated fibroblasts. Cancer Discov. 9, 1102-1123 (2019).
55. Moncada, R. et al. Integrating microarray-based spatial transcriptomics and single-cell RNA-seq reveals tissue architecture in pancreatic ductal adenocarcinomas. Nat. Biotechnol. 38, 333-342 (2020).
56. Peng, J. et al. Single-cell RNA-seq highlights intra-tumoral heterogeneity and malignant progression in pancreatic ductal adenocarcinoma. Cell Res. 29, 725-738 (2019).
57. De La 0, J. P. et al. Notch and Kras reprogram pancreatic acinar cells to ductal intraepithelial neoplasia. Proc. Natl. Acad. Sci. U.S.A 105, (2008).
58. Guerra, C. et al. Chronic Pancreatitis Is Essential for Induction of Pancreatic Ductal Adenocarcinoma by K-Ras Oncogenes in Adult Mice. Cancer Cell 11, (2007).
59. Morris I V, J. P., Cano, D. A., Sekine, S., Wang, S. C. & Hebrok, M. β-catenin blocks Kras-dependent reprogramming of acini into pancreatic cancer precursor lesions in mice. J. Clin. Invest. 120, (2010).
60. Liberzon, A. et al. The Molecular Signatures Database Hallmark Gene Set Collection. Cell Syst. 1, 417-425 (2015).
61. Li, Q. et al. Reg proteins promote acinar-to-ductal metaplasia and act as novel diagnostic and prognostic markers in pancreatic ductal adenocarcinoma. Oncotarget 7, 77838-77853 (2016).
62. Liu, X. et al. REG3A accelerates pancreatic cancer cell growth under IL-6-associated inflammatory condition: Involvement of a REG3A-JAK2/STAT3 positive feedback loop. Cancer Lett. 362, 45-60 (2015).
63. Roa-Peñ a, L. et al. Keratin 17 identifies the most lethal molecular subtype of pancreatic cancer. Sci. Rep. 9, (2019).
64. Wolf, F. A. et al. PAGA: graph abstraction reconciles clustering with trajectory inference through a topology preserving map of single cells. Genome Biol. 20, 59 (2019).
65. Wherry, E. J. et al. Molecular Signature of CD8+ T Cell Exhaustion during Chronic Viral Infection. Immunity 27, 670-684 (2007).
66. Eberlein, J. et al. Chemokine Signatures of Pathogen-Specific T Cells I: Effector T Cells. J. Immunol. 205, 2169-2187 (2020).
67. Johnston, R. J. et al. The Immunoreceptor TIGIT Regulates Antitumor and Antiviral CD8+ T Cell Effector Function. Cancer Cell 26, 923-937 (2014).
68. Chauvin, J.-M. et al. TIGIT and PD-1 impair tumor antigen-specific CD8+ T cells in melanoma patients. J. Clin. Invest. 125, 2046-2058 (2015).
69. Zhou, X. et al. Differentiation and Persistence of Memory CD8+ T Cells Depend on T Cell Factor 1. Immunity 33, 229-240 (2010).
70. Im, S. J. et al. Defining CD8+ T cells that provide the proliferative burst after PD-1 therapy. Nature 537, 417-421 (2016).
71. Siddiqui, I. et al. Intratumoral Tcfl+PD-1+CD8+ T Cells with Stem-like Properties Promote Tumor Control in Response to Vaccination and Checkpoint Blockade Immunotherapy. Immunity 50, 195-211.e10 (2019).
72. Carlson, C. M. et al. Kruppel-like factor 2 regulates thymocyte and T-cell migration. Nature 442, 299-302 (2006).
73. Willinger, T. et al. Human Naive CD8 T Cells Down-Regulate Expression of the WNT Pathway Transcription Factors Lymphoid Enhancer Binding Factor 1 and Transcription Factor 7 (T Cell Factor-1) following Antigen Encounter In Vitro and In Vivo. J. Immunol. 176, 1439-1446 (2006).
74. Incio, J. et al. Obesity-Induced Inflammation and Desmoplasia Promote Pancreatic Cancer Progression and Resistance to Chemotherapy. Cancer Discov. 6, 852-869 (2016).
75. Juiz, N. et al. Basal-like and classical cells coexist in pancreatic cancer revealed by single-cell analysis on biopsy-derived pancreatic cancer organoids from the classical subtype. FASEB J. 34, 12214-12228 (2020).
76. Chan-Seng-Yue, M. et al. Transcription phenotypes of pancreatic cancer are driven by genomic events during tumor evolution. Nat. Genet. 52, 231-240 (2020).
77. Raghavan, S. et al. The tumor microenvironment drives transcriptional phenotypes and their plasticity in metastatic pancreatic cancer. bioRxiv 6, 2020.08.25.256214 (2021).
78. Öhlund, D. et al. Distinct populations of inflammatory fibroblasts and myofibroblasts in pancreatic cancer. J. Exp. Med. 214, 579⁻⁵⁹⁶ (2017).
79. Kotliar, D. et al. Identifying gene expression programs of cell-type identity and cellular activity with. Elife 8, e43803 (2019).
80. He, H. et al. Krüppel-like Factor 4 Promotes Esophageal Squamous Cell Carcinoma Differentiation by Up-regulating Keratin 13 Expression. J. Biol. Chem. 290, 13567-13577 (2015).
81. Rorke, E. A. et al. Structural and biochemical changes underlying a keratoderma-like phenotype in mice lacking suprabasal AP1 transcription factor function. Cell Death Dis. 6, e1647 (2015).
82. Champliaud, M. F. et al. Gene characterization of sciellin (SCEL) and protein localization in vertebrate epithelia displaying barrier properties. Genomics 70, 264-268 (2000).
83. Zhang, D. et al. Stem cell and neurogenic gene-expression profiles link prostate basal cells to aggressive prostate cancer. Nat. Commun. 7, (2016).
84. Karasawa, M. et al. Localization of metallothionein in hair follicles of normal skin and the basal cell layer of hyperplastic epidermis: Possible association with cell proliferation. J. Invest. Dermatol. 97, 97-100 (1991).
85. Beuran, M. et al. The epithelial to mesenchymal transition in pancreatic cancer: A systematic review. Pancreatology 15, 217-225 (2015).
86. Aiello, N. M. et al. EMT Subtype Influences Epithelial Plasticity and Mode of Cell Migration. Dev. Cell 45, 681-695 (2018).
87. Dardenne, E. et al. N-Myc Induces an EZH2-Mediated Transcriptional Program Driving Neuroendocrine Prostate Cancer. Cancer Cell 30, (2016).
88. Da Costa, J. B. et al. Molecular characterization of neuroendocrinelike bladder cancer. Clin. Cancer Res. 25, 3908-3920 (2019).
89. Qiu, H. et al. MEN1 deficiency leads to neuroendocrine differentiation of lung cancer and disrupts the DNA damage response. Nat. Commun. 11, 1009 (2020).
90. Wasser, C. R. & Herz, J. Reelin: Neurodevelopmental Architect and Homeostatic Regulator of Excitatory Synapses. J. Biol. Chem. 292, 1330-1338 (2017).
91. Chauvet, S. et al. Gating of Sema3E/PlexinD1 signaling by neuropilin-1 switches axonal repulsion to attraction during brain development. Neuron 56, 807-822 (2007).
92. Nakamura, F. & Goshima, Y. Structural and functional relation of neuropilins. Adv. Exp. Med. Biol. 515, 55-69 (2002).
93. Brinkmann, B. G. et al. Neuregulin-1/ErbB signaling serves distinct functions in myelination of the peripheral and central nervous system. Neuron 59, 581-595 (2008).
94. Zahalka, A. H. & Frenette, P. S. Nerves in cancer. Nat. Rev. Cancer 20, 143-157 (2020).
95. Liebig, C., Ayala, G., Wilks, J. A., Berger, D. H. & Albo, D. Perineural invasion in cancer: A review of the literature. Cancer 115, 3379-3391 (2009).
96. Guo, J. A. et al. Pan-cancer Transcriptomic Predictors of Perineural Invasion Improve Occult Histopathologic Detection. Clin. Cancer Res. 27, 2807-2815 (2021).
97. Robertson, A. G. et al. Comprehensive Molecular Characterization of Muscle-Invasive Bladder Cancer. Cell 171, 540-556 (2017).
98. Sjödahl, G., Eriksson, P., Liedberg, F. & Höglund, M. Molecular classification of urothelial carcinoma: global mRNA classification versus tumour-cell phenotype classification. J. Pathol. 242, 113-125 (2017).
99. Marzouka, N. A. D. et al. A validation and extended description of the Lund taxonomy for urothelial carcinoma using the TCGA cohort. Sci. Rep. 8, 3737 (2018).
100. Morioka, K. Degeneration of ribosomes in keratinizing cells: Possible participation of RNase A in the skin and hair of rat. Acta Histochem. Cytochem. 37, 399-406 (2004).
101. Ligorio, M. et al. Stromal Microenvironment Shapes the Intratumoral Architecture of Pancreatic Cancer. Cell 178, 160-175 (2019).
102. Farrell, A. S. et al. MYC regulates ductal-neuroendocrine lineage plasticity in pancreatic ductal adenocarcinoma associated with poor outcome and chemoresistance. Nat. Commun. 8, 1-12 (2017).
103. Tanaka, Y. et al. Circulation-Independent Differentiation Pathway from Extraembryonic Mesoderm toward Hematopoietic Stem Cells via Hemogenic Angioblasts. Cell Rep. 8, 31-39 (2014).
104. Komori, T. Runx2, a multifunctional transcription factor in skeletal development. J Cell. Biochem. 87, 1-8 (2002).
105. Roel, G., YY, G., J, P.-M., FJ, V. & O, D. Lefl plays a role in patterning the mesoderm and ectoderm in Xenopus tropicalis. Int. J. Dev. Biol. 53, 81-89 (2009).
106. Tahara, N. et al. Sa114 regulates neuromesodermal progenitors and their descendants during body elongation in mouse embryos. Development 146, dev177659 (2019).
107. Andre, P., Song, H., Kim, W., Kispert, A. & Yang, Y. Wnt5a and Wnt11 regulate mammalian anterior-posterior axis elongation. Development 142, 1516-1527 (2015).
108. Ishikawa, A. et al. Mouse Nkd1, a Wnt antagonist, exhibits oscillatory gene expression in the PSM under the control of Notch signaling. Mech. Dev. 121, 1443-1453 (2004).
109. Cherubini, A. et al. FOXP1 circular RNA sustains mesenchymal stem cell identity via microRNA inhibition. Nucleic Acids Res. 47, 5325-5340 (2019).
110. Adams, K. L., Rousso, D. L., Umbach, J. A. & Novitch, B. G. Foxp1-mediated programming of limb-innervating motor neurons from mouse and human embryonic stem cells. Nat. Commun. 6, 1-16 (2015).
111. Buechler, M. B. et al. Cross-tissue organization of the fibroblast lineage. Nature 593, 575-579 (2021).
112. Sanchez-Martin, L. et al. The chemokine CXCL12 regulates monocyte-macrophage differentiation and RUNX3 expression. Blood 117, 88-97 (2011).
113. Sleightholm, R. L. et al. Emerging roles of the CXCL12/CXCR4 axis in pancreatic cancer progression and therapy. Pharmacol. Ther. 179, 158-170 (2017).
114. Cheng, H. W. et al. CCL19-producing fibroblastic stromal cells restrain lung carcinoma growth by promoting local antitumor T-cell responses. J. Allergy Clin. Immunol. 142, 1257-1271 (2018).
115. Hirth, M. et al. CXCL10 and CCL21 Promote Migration of Pancreatic Cancer Cells Toward Sensory Neurons and Neural Remodeling in Tumors in Mice, Associated With Pain in Patients. Gastroenterology 159, 665-681 (2020).
116. Boulakirba, S. et al. IL-34 and CSF-1 display an equivalent macrophage differentiation ability but a different polarization potential. Sci. Rep. 8, 256 (2018).
117. Bettac, L., Denk, S., Seufferlein, T. & Huber-Lang, M. Complement in pancreatic disease-Perpetrator or savior? Front. Immunol. 8, 15 (2017).
118. Zhang, R., Liu, Q., Li, T., Liao, Q. & Zhao, Y. Role of the complement system in the tumor microenvironment. Cancer Cell Int. 19, 300 (2019).
119. Jurcak, N. & Zheng, L. Signaling in the microenvironment of pancreatic cancer: Transmitting along the nerve. Pharmacol. Ther. 200, 126-134 (2019).
120. Puleo, F. et al. Stratification of Pancreatic Ductal Adenocarcinomas Based on Tumor and Microenvironment Features. Gastroenterology 155, 1999-2013 (2018).
121. Somerville, T. D. D. et al. Squamous trans-differentiation of pancreatic cancer cells promotes stromal inflammation. Elife 9, e53381 (2020).
122. Karamitopoulou, E. Tumour microenvironment of pancreatic cancer: immune landscape is dictated by molecular and histopathological features. Br. J. Cancer 121, 5-14 (2019).
123. Merritt, C. R. et al. Multiplex digital spatial profiling of proteins and RNA in fixed tissue. Nat. Biotechnol. 38, 586⁻⁵⁹⁹ (2020).
124. Mutgan, A. C. et al. Insulin/IGF-driven cancer cell-stroma crosstalk as a novel therapeutic target in pancreatic cancer. Mol. Cancer 17, 66 (2018).
125. Biasci, D. et al. CXCR4 inhibition in human pancreatic and colorectal cancers induces an integrated immune response. Proc. Natl. Acad. Sci. U.S.A 117, 28960-28970 (2020).
126. Fielder, G. C. et al. The GDNF Family: A Role in Cancer? Neoplasia 20, 99-117 (2018).
127. Mulligan, L. M. GDNF and the RET Receptor in Cancer: New Insights and Therapeutic Potential. Front. Physiol. 9, 1873 (2019).
128. Slebioda, T. J. et al. Triggering of TNFRSF25 promotes CD8+ T-cell responses and anti-tumor immunity. Eur. J. Immunol. 41, 2606-2611 (2011).
129. Cheng, Y., Ma, X. lei, Wei, Y. quan & Wei, X. W. Potential roles and targeted therapy of the CXCLs/CXCR2 axis in cancer and inflammatory diseases. Biochim. Biophys. Acta -Rev. Cancer 1871, 289-312 (2019).
130. Tjomsland, V. et al. The desmoplastic stroma plays an essential role in the accumulation and modulation of infiltrated immune cells in pancreatic adenocarcinoma. Clin. Dev. Immunol. 212810 (2011) doi:10.1155/2011/212810.
131. Erez, N., Truitt, M., Olson, P. & Hanahan, D. Cancer-Associated Fibroblasts Are Activated in Incipient Neoplasia to Orchestrate Tumor-Promoting Inflammation in an NF-κB-Dependent Manner. Cancer Cell 17, 135-147 (2010).
132. Forster, R., Davalos-Misslitz, A. C. & Rot, A. CCR7 and its ligands: balancing immunity and tolerance. Nat. Rev. Immunol. 8, 362-371 (2008).
133. Sautés-Fridman, C., Petitprez, F., Calderaro, J. & Fridman, W. Tertiary lymphoid structures in the era of cancer immunotherapy. Nat. Rev. Cancer 19, 307-325 (2019).
134. Edwardson, D. W. et al. Inflammatory cytokine production in tumor cells upon chemotherapy drug exposure or upon selection for drug resistance. PLoS One 12, e0183662 (2017).
135. Tjomsland, V. et al. Interleukin la sustains the expression of inflammatory factors in human pancreatic cancer microenvironment by targeting cancer-associated fibroblasts. Neoplasia 13, 664-675 (2011).
136. Steele, C. W. et al. CXCR2 Inhibition Profoundly Suppresses Metastases and Augments Immunotherapy in Pancreatic Ductal Adenocarcinoma. Cancer Cell 29, 832-845 (2016).
137. Mulvihill, E. E. & Drucker, D. J. Pharmacology, physiology, and mechanisms of action of dipeptidyl peptidase-4 inhibitors. Endocr. Rev. 35, 992-1019 (2014).
138. Waldmann, J. et al. Expression of neuropeptide Y and its receptors Y1 and Y2 in pancreatic intraepithelial neoplasia and invasive pancreatic cancer in a transgenic mouse model and human samples of pancreatic cancer. J. Surg. Res. 223, 230-236 (2018).
139. Skrypek, N. et al. The oncogenic receptor ErbB2 modulates gemcitabine and irinotecan/SN-38 chemoresistance of human pancreatic cancer cells via hCNT1 transporter and multidrug-resistance associated protein MRP-2. Oncotarget 6, 10853-10867 (2015).
140. Holmes, W. et al. Identification of heregulin, a specific activator of p185erbB2. Science (80-.). 256, 1205-1210 (1992).
141. Iwamoto, R. et al. Heparin-binding EGF-like growth factor and ErbB signaling is essential for heart function. Proc. Natl. Acad. Sci. U.S.A 100, 3221-3226 (2003).
142. Donier, E. et al. L1 CAM binds ErbB receptors through Ig-like domains coupling cell adhesion and neuregulin signalling. PLoS One 7, e40674 (2012).
143. Fock, V. et al. Neuregulin-1-mediated ErbB2-ErbB3 signalling protects human trophoblasts against apoptosis to preserve differentiation. J. Cell Sci. 128, 4306-4316 (2015).
144. Shouval, D. S. et al. Interleukin 10 Receptor Signaling: Master Regulator of Intestinal Mucosal Homeostasis in Mice and Humans. Adv. Immunol. 122, 177-210 (2014).
145. Guo, Y. et al. Metabolic reprogramming of terminally exhausted CD8+ T cells by IL-10 enhances anti-tumor immunity. Nat. Immunol. 2021 226 22, 746-756 (2021).
146. Storz, P. Acinar cell plasticity and development of pancreatic ductal adenocarcinoma. Nat. Rev. Gastroenterol. Hepatol. 14, 296-304 (2017).
147. Maji, S. et al. Bcl-2 Antiapoptotic Family Proteins and Chemoresistance in Cancer. Adv. Cancer Res. 137, 37-75 (2018).
148. Zhang, M. et al. Platelet-derived growth Factor D is a prognostic biomarker and is associated with platinum resistance in epithelial ovarian cancer. Int. J. Gynecol. Cancer 28, 323-331 (2018).
149. Chen, X. et al. SPP1 inhibition improves the cisplatin chemo-sensitivity of cervical cancer cell lines. Cancer Chemother. Pharmacol. 83, 603-613 (2019).
150. Wu, Q. et al. Chemoresistance to gemcitabine in hepatoma cells induces epithelial-mesenchymal transition and involves activation of PDGF-D pathway. Oncotarget 4, 1999-2009 (2013).
151. Lok, S. W. et al. A phase Ib dose-escalation and expansion study of the BCL2 inhibitor venetoclax combined with tamoxifen in ER and BCL2—positive metastatic breast cancer. Cancer Discov. 9, 354-369 (2019).
152. Chen, Y.-A., Lu, I.-L. & Tsai, J.-W. Contactin-1/F3 Regulates Neuronal Migration and Morphogenesis Through Modulating RhoA Activity. Front. Mol. Neurosci. 11, 422 (2018).
153. Donato, V. Di et al. An Attractive Reelin Gradient Establishes Synaptic Lamination in the Vertebrate Visual System. Neuron 97, 1049-1062 (2018).
154. Telley, L. et al. Dual Function of NRP1 in Axon Guidance and Subcellular Target Recognition in Cerebellum Article Dual Function of NRP1 in Axon Guidance and Subcellular Target Recognition in Cerebellum. Neuron 91, 1276-1291 (2016).
155. Meng, H. et al. DCDC2 is associated with reading disability and modulates neuronal development in the brain. Proc. Natl. Acad. Sci. U.S.A 102, 17053-17058 (2005).
156. Kantor, D. B. et al. Semaphorin 5A Is a Bifunctional Axon Guidance Cue Regulated by Heparan and Chondroitin Sulfate Proteoglycans. Neuron 44, 961-975 (2004).
157. Schmid, R. S. & Maness, P. F. L1 and NCAM adhesion molecules as signaling co-receptors in neuronal migration and process outgrowth. Curr. Opin. Neurobiol. 18, 245-250 (2008).
158. Pang, W. et al. Untangle the Multi-Facet Functions of Auts2 as an Entry Point to Understand Neurodevelopmental Disorders. Front. Psychiatry 12, 580433 (2021).
159. Banh, R. S. et al. Neurons release serine to support mRNA translation in pancreatic cancer. Cell 183, 1202-1218 (2020).
160. Porter, R. L. et al. Epithelial to mesenchymal plasticity and differential response to therapies in pancreatic ductal adenocarcinoma. Proc. Natl. Acad. Sci. U.S.A 116, 26835-26845 (2019).
161. Exner, S. et al. Agtrl is overexpressed in neuroendocrine neoplasms, regulates secretion and may potentially serve as a target for molecular imaging and therapy. Cancers (Basel). 12, 3138 (2020).
162. Oren, Y. et al. Cycling cancer persister cells arise from lineages with distinct programs. Nat. 2021 15, 1-7 (2021).
163. Feig, C. et al. Targeting CXCL12 from FAP-expressing carcinoma-associated fibroblasts synergizes with anti-PD-L1 immunotherapy in pancreatic cancer. Proc. Natl. Acad. Sci. U.S.A 110, 20212-20217 (2013).
164. Fearon, D. T. & Janowitz, T. AMD3100/Plerixafor overcomes immune inhibition by the CXCL12-KRT19 coating on pancreatic and colorectal cancer cells. Br. J Cancer 125, 149-151 (2021).
165. Harder, J. et al. Multicentre phase II trial of trastuzumab and capecitabine in patients with HER2 overexpressing metastatic pancreatic cancer. Br. J. Cancer 106, 1033-1038 (2012).
166. Safran, H. et al. Lapatinib and gemcitabine for metastatic pancreatic cancer. A phase II study. Am. J Clin. Oncol. 34, 50-52 (2011).
167. Sjoquist, K. M. et al. Personalising pancreas cancer treatment: When tissue is the issue. World J. Gastroenterol. 20, 7849-7863 (2014).
168. Chantrill, L. A. et al. Precision Medicine for Advanced Pancreas Cancer: The Individualized Molecular Pancreatic Cancer Therapy (IMPaCT) Trial. Clin. Cancer Res. 21, 2029-2037 (2015).
169. Fleming, S. J., Marioni, J. C. & Babadi, M. CellBender remove-background: A deep generative model for unsupervised removal of background noise from scRNA-seq datasets. bioRxiv (2019) doi:10.1101/791699.
170. Wolf, F. A., Angerer, P. & Theis, F. J. SCANPY: Large-scale single-cell gene expression data analysis. Genome Biol. 19, 15 (2018).
171. Korsunsky, I. et al. Fast, sensitive and accurate integration of single-cell data with Harmony. Nat. Methods 16, 1289-1296 (2019).
172. Traag, V. A., Waltman, L. & van Eck, N. J. From Louvain to Leiden: guaranteeing well-connected communities. Sci. Rep. 9, 1-12 (2019).
173. McInnes, L., Healy, J. & Melville, J. UMAP: Uniform Manifold Approximation and Projection for Dimension Reduction. arXiv (2018).
174. Büttner, M., Ostner, J., Muller, C., Theis, F. & Schubert, B. scCODA: A Bayesian model for compositional single-cell data analysis. bioRxiv 12.14.422688 (2020) doi:10.1101/2020.12.14.422688.
175. Schapiro, D. et al. HistoCAT: Analysis of cell phenotypes and interactions in multiplex image cytometry data. Nat. Methods 14, 873-876 (2017).
176. Zanotelli, V. R. R. T. & Bodenmiller, B. ImcSegmentationPipeline: A pixelclassification based multiplexed image segmentation pipeline. (Zenodo, 2017). doi:10.5281/zenodo.3841961.
177. Zanotelli, V., Ndamond & Strotton, M. BodenmillerGroup/ImcSegmentationPipeline: IMC Segmentation Pipeline. (2020) doi:http://doi.org/10.5281/zenodo.3841961.
178. Berg, S. et al. ilastik: interactive machine learning for (bio)image analysis. Nat. Methods 16, 1226-1232 (2019).
179. McQuin, C. et al. CellProfiler 3.0: Next-generation image processing for biology. PLoS Biol. 16, e2005970 (2018).
180. Lopez, R. et al. A joint model of unpaired data from scRNA-seq and spatial transcriptomics for imputing missing gene expression measurements. arXiv 1905.02269 (2019).
181. Muus, C. et al. Single-cell meta-analysis of SARS-CoV-2 entry genes across tissues and demographics. Nat. Med. 27, 546⁻⁵⁵⁹ (2021).
182. Subramanian, A. et al. Gene set enrichment analysis: A knowledge-based approach for interpreting genome-wide expression profiles. Proc. Natl. Acad. Sci. U.S.A 102, 15545-15550 (2005).
183. Li, B. & Dewey, C. N. RSEM: Accurate transcript quantification from RNA-Seq data with or without a reference genome. BMC Bioinformatics 12, 323 (2011).
184. Uhlén, M. et al. Tissue-based map of the human proteome. Science (80-.). 347, 1260419 (2015).
185. Hanzelmann, S., Castelo, R. & Guinney, J. GSVA: Gene set variation analysis for microarray and RNA-Seq data. BMC Bioinformatics 14, 7 (2013).
186. Bates, D., Mächler, M., Bolker, B. M. & Walker, S. C. Fitting linear mixed-effects models using lme4. J. Stat. Softw. 67, 1-48 (2015).
187. Kuznetsova, A., Brockhoff, P. B. & Christensen, R. H. B. lmerTest Package: Tests in Linear Mixed Effects Models. J. Stat. Softw. 82, 1-26 (2017).
188. Benjamini, Y. & Hochberg, Y. Controlling the False Discovery Rate: A Practical and Powerful Approach to Multiple Testing. J. R. Stat. Soc. Ser. B 57, 289-300 (1995).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of stratifying pancreatic ductal adenocarcinoma (PDAC) patients into treatment groups and/or prognosing PDAC or treatment outcome and/or survival in a patient comprising:
detecting, in one or more PDAC tumor cells from a PDAC tumor,
a. a malignant cell signature, program, or both;
b. a cancer-associated fibroblast (CAF) signature, program, or both;
c. an immune microniche signature, program, or both;
d. a tumor spatial neighborhood;
e. one or more co-expressed receptor-ligand pairs; or
f. any combination thereof,
wherein a characteristic regarding a patient's treatment, a patient's response to a treatment, and/or their survival is determined or predicted based on the detection of one or more of the signatures, programs, and/or or states.

2. The method of claim 1, wherein the malignant cell signature and/or program comprises:
a. a lineage specific expression program selected from: a squamous program, a mesenchymal cytoskeletal program, mesenchymal matrisomal program, a classical progenitor program, a classical activated program, or any combination thereof;
b. a lineage specific expression program selected from: a squamous program, a mesenchymal program, an induced basal-like program, a classical progenitor program, a classical acinar-like program, a classical neuroendocrine-like program, or any combination thereof,
c. a cell state specific expression program selected from: a cycling program, a hypoxic program, TNF-NFkB signaling program, an interferon signaling program, or any combination thereof,
d. a cell state specific expression program selected from: a cycling program, a TNF-NFkB signaling program, or an interferon signaling program, or any combination thereof;
e. a neoadjuvant treated malignant cell expression program;
f. an untreated malignant cell expression program;
g. a cell state expression program selected from: a neuronal-like program, a neuroendocrine like program, a mesenchymal program, a squamoid program, a MYC signaling program, a cycling (G2M) program, a cycling (S) program, or any combination thereof;
h. a lineage specific expression program selected from: an acinar-like program, a classical-like program, a basaloid program, a squamoid program, a mesenchymal program, a neuroendocrine like program, a neuronal like program, or any combination thereof;
i. or any combination thereof
optionally wherein a subject having a classical-like malignant expression program has the greatest likelihood of time to progression and longest survival; and
optionally wherein a subject having a neuronal like malignant expression program or a malignant squamoid expression program has the greatest likelihood of least time to progression.

3. The method of claim 1, wherein the CAF signature and/or program:
a. comprises a myofibroblast program; a neurotropic program; a secretory program;
a mesodermal progenitor program a neuromuscular program; or any combination thereof;
b. comprises a neoadjuvant treated CAF signature and/or program selected from: a neuromuscular program, a secretory program, a neurotropic program, or any combination thereof;
c. comprises an untreated CAF signature and/or program selected from: a mesodermal progenitor program, a myofibroblast program, a neurotropic program, a secretory program, or any combination thereof; or
d. comprises an adhesive expression program, an immunomodulatory expression program, a myofibroblastic progenitor expression program, or a neurotropic expression program,
optionally wherein a subject having an immunomodulatory CAF expression program has the greatest likelihood of time to progression;
optionally wherein a subject having an adhesive CAF expression program has the greatest likelihood of shortest survival; and
optionally wherein there is a greater likelihood of longer survival when a CAF secretory or neurotropic program is detected as compared to detection of a myofibroblast or mesodermal progenitor program is detected.

4. The method of claim 1, wherein the tumor spatial neighborhood is
a. a treatment enriched neighborhood
b. a squamoid-basaloid neighborhood; or a
c. a classical neighborhood.

5. The method of claim 1, wherein the one or more co-expressed receptor-ligand pairs is selected from:
a. an Epithelial compartment—CAF compartment pair;
b. an Epithelial compartment—Immune compartment pair;
c. a CAF compartment and Immune compartment pair;
d. or any combination thereof.

6. The method of claim 1, wherein the method comprises, detecting, in one or more a PDAC tumor cells, an untreated tumor malignant cell signature and/or program and an untreated CAF signature and/or program, wherein
a. the untreated tumor malignant cell signature and/or program comprises a lineage specific expression program selected from: a squamous program, a mesenchymal cytoskeletal program, mesenchymal matrisomal program, a classical progenitor program, a classical activated program, or any combination thereof; and
b. the untreated tumor CAF signature and/or program is selected from: a mesodermal progenitor program, a myofibroblast program, a neurotropic program, a secretory program, or any combination thereof
optionally further comprising determining a tumor heterogeneity score for the PDAC tumor, wherein the tumor heterogeneity score is calculated by determining a number of highly expressed programs in the one or more PDAC cells;
optionally further comprising assigning the PDAC tumor to a single malignant class and to a single CAF class, wherein the malignant class is selected from A0, A1, A2, S0, S1, S2, C0, C1, C2, M0, M1, M2, P0, P1, or P2, and wherein the CAF class is selected from S0, S1, N0, N1, M0, M1, P0, or P1;
optionally wherein the PDAC tumor is assigned to a combined risk class that integrates the malignant risk group and CAF risk group class and is selected from: a low combined risk group, a low-intermediate combined risk group, a high-intermediate risk group, or a high combined risk group, wherein
a. a PDAC tumor in a low malignant risk group and in a low CAF risk group is classified into the low combined risk group;
b. a PDAC tumor in a high malignant risk and in a high CAF risk is classified into the high combined risk group;
c. a PDAC tumor in an intermediate malignant risk group or in an intermediate CAF risk and in a high malignant risk or in a high CAF risk is classified into the high-intermediate combined risk group; and
d. a PDAC tumor in a low malignant risk group and in a high CAF risk group, a PDAC tumor in a high malignant risk group and in a low CAF risk group, a PDAC tumor in a low malignant risk group and in a low CAF risk group, a PDAC tumor in an intermediate malignant risk group and in an intermediate CAF risk group, a PDAC tumor in a low malignant risk group and in an intermediate CAF risk group is classified into the low-intermediate combined risk group; and
optionally wherein a subject with a PDAC tumor in low combined risk group has the greatest likelihood of longest survival.

7.-14. (canceled)

15. The method of claim 1, wherein the malignant cell signature and/or program comprises one or more biomarkers, expression programs, biologic programs, receptor-ligand interactions, cell state distribution, cell type distribution, or any combination thereof as in any of 1B-1D, 2A-2D, 3A-3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

16. The method of claim 1, wherein the CAF cell signature comprises one or more biomarkers, expression programs, biologic programs, receptor-ligand interactions, cell state distribution, cell type distribution, or any combination thereof as in any of FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

17. The method of claim 1, wherein the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, Table 7, or any combination thereof.

18. The method of claim 1, wherein there is a greater likelihood of longer survival when a predominant mesenchymal matrisomal and/or a classical progenitor malignant program is detected as compared to detection of a primary classical activated, a squamous, or a mesenchymal cytoskeletal program.

19. (canceled)

20. The method of claim 1, wherein the PDAC patient had or is concurrently receiving a neoadjuvant therapy.

21. The method of claim 1, wherein detecting comprises a single cell RNA sequencing technique.

22. The method of claim 1, wherein detecting comprises a single-nucleus RNA sequencing technique,
optionally wherein the single-nucleus RNA sequencing technique is optimized for pancreatic tissue;
optionally wherein the single-nucleus RNA sequencing technique is optimized for frozen samples; and
optionally wherein the single-nucleus RNA sequencing technique comprises screening a sample for an RNA integrity number and performing single nucleus RNA sequencing only on samples with an RNA integrity number of 6 or more.

23.-25. (canceled)

26. The method of claim 1, wherein detecting comprises a spatially-resolved transcriptomics technique.

27. A method of treating pancreatic ductal adenocarcinoma (PDAC) in a subject in need thereof comprising:
a. preventing a shift in the state of a malignant cell from a classical progenitor state to a basal-like state or a terminally-differentiated state;
b. modulating a cell state of a malignant cell from a basal-like state or a terminally-differentiated state to a classical progenitor state;
c. inhibiting, preventing, or modulating expression of a neuronal like expression program in a malignant cells;
d. inhibiting, preventing expression or modulating expression of a malignant squamoid expression program in a malignant cell,
e. inhibiting, preventing, or modulating expression of an adhesive CAF expression program in a CAF cell; or
f. any combination thereof, or
g. administering a neoadjuvant therapy to the subject; and administering a PDAC malignant cell modulating agent, an immune modulator, a CAF modulating agent, an apoptosis inhibitor, a myeloid cell agonist, a TGFbeta modulator, a CXCR4 inhibitor, a HER2 inhibitor, or any combination thereof to the subject.

28. The method of claim 27, wherein the subject has had neoadjuvant therapy, is concurrently receiving or undergoing neoadjuvant therapy or wherein the subject has not had neoadjuvant therapy.

29. The method of claim 27, a malignant cell state is characterized by a malignant cell signature and/or program comprising:
a. a lineage specific expression program selected from: a squamous program, a mesenchymal cytoskeletal program, mesenchymal matrisomal program, a classical progenitor program, or a classical activated program;
b. a lineage specific expression program selected from: a squamous program, a mesenchymal program, an induced basal-like program, a classical progenitor program, a classical acinar-like program, and a classical neuroendocrine-like program;
c. a cell state specific expression program selected from: a cycling program, a hypoxic program, TNF-NFkB signaling program, or an interferon signaling program;
d. a cell state specific expression program selected from: a cycling program, a TNF-NFkB signaling program, or an interferon signaling program;
e. a neoadjuvant treated malignant cell expression program;
f. an untreated malignant cell expression program;
g. a basal-like malignant cell expression program;
h. a classic-like malignant cell expression program;
i. an immune microniche signature;
j. a cell state expression program selected from: a neuronal-like program, a neuroendocrine like program, a mesenchymal program, a squamoid program, a MYC signaling program, a cycling (G2M) program, a cycling (S) program, or any combination thereof; or
k. a lineage specific expression program selected from: an acinar-like program, a classical-like program, a basaloid program, a squamoid program, a mesenchymal program, a neuroendocrine like program, a neuronal like program, or any combination thereof; or
l. any combination thereof,
optionally wherein the malignant cell signature comprises one or more biomarkers, expression programs, biologic programs, receptor-ligand interactions, cell state distribution, cell type distribution, or any combination thereof as in any of 1B-1D, 2A-2D, 3A-3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof;
optionally wherein modulating the cell state comprises reducing the distance in gene expression space between the basal-like malignant cell state and the classic-like malignant cell states;
optionally wherein the gene expression spaces comprises 10 or more genes, 20 or more genes, 30 or more genes, 40 or more genes, 50 or more genes, 100 or more genes, 500 or more genes, or 1000 or more genes;
optionally wherein the distance is measured by a Euclidean distance, Pearson coefficient, Spearman coefficient, or combination thereof;
optionally wherein modulation comprises increasing or decreasing expression of one or more genes, gene expression cassettes, or gene expression signatures; and
optionally wherein the modulating agent comprises a therapeutic antibody or fragment thereof, antibody-like protein scaffold, aptamer, polypeptide, a polynucleotide, a genetic modifying agent or system, a small molecule therapeutic, a chemotherapeutic, small molecule degrader, inhibitor, an immunomodulator, or a combination thereof.

30.-34. (canceled)

35. The method of treating PDAC in a subject in need thereof as in claim 27, wherein modulating or preventing comprises administering a modulating agent to the subject.

36. (canceled)

37. A method of screening for one or more agents capable of modulating a PDAC malignant cell state comprising:
contacting a cell population comprising PDAC malignant cells having an initial cell state with a test modulating agent or library of modulating agents;
determining a fraction of malignant cells having a desired cell state and an undesired cell state;
selecting modulating agents that shift the initial PDAC malignant cell state to a desired cell state or prevent the initial PDAC malignant cell state to shift from a desired initial state, such that the fraction of PDAC malignant cells in the cell population having a desired cell state is above a set cutoff limit,
optionally wherein the desired PDAC malignant cell state is a classic progenitor cell state or a mesenchymal matrisomal cell state; and
optionally wherein the cell population is obtained from a subject to be treated.

38.-40. (canceled)

41. A method of treating a subject having PDAC, the method comprising: detecting, in one or more PDAC tumor cells,
a. a malignant cell signature, program, or both;
b. a cancer-associated fibroblast (CAF) signature, program, or both;
c. an immune microniche signature, program, or both;
d. a tumor spatial neighborhood;
e. one or more co-expressed receptor-ligand pairs; or
f. any combination thereof, and
administering or applying a PDAC treatment to the subject in need thereof, wherein the treatment is optionally a tumor resection, a chemotherapy, a radiation therapy, a neoadjuvant, a malignant cell signature and/or program modulating agent, a BCL-2 inhibitor, a tyrosine kinase inhibitor, a TGFbeta modulator, a myeloid cell agonist, a CXCR4 inhibitor, a HER2 inhibitor, or any combination thereof.

42. The method of claim 41, wherein the wherein the malignant cell signature and/or program comprises
a. a lineage specific expression program selected from: a squamous program, a mesenchymal cytoskeletal program, mesenchymal matrisomal program, a classical progenitor program, a classical activated program, or any combination thereof;
b. a lineage specific expression program selected from: a squamous program, a mesenchymal program, an induced basal-like program, a classical progenitor program, a classical acinar-like program, a classical neuroendocrine-like program, or any combination thereof,
c. a cell state specific expression program selected from: a cycling program, a hypoxic program, TNF-NFkB signaling program, an interferon signaling program, or any combination thereof,
d. a cell state specific expression program selected from: a cycling program, a TNF-NFkB signaling program, or an interferon signaling program, or any combination thereof;
e. a neoadjuvant treated malignant cell expression program;
f. an untreated malignant cell expression program;
g. a cell state expression program selected from: a neuronal-like program, a neuroendocrine like program, a mesenchymal program, a squamoid program, a MYC signaling program, a cycling (G2M) program, a cycling (S) program, or any combination thereof; or
h. a lineage specific expression program selected from: an acinar-like program, a classical-like program, a basaloid program, a squamoid program, a mesenchymal program, a neuroendocrine like program, a neuronal like program, or any combination thereof;
i. or any combination thereof
optionally wherein a subject having a classical-like malignant expression program has the greatest likelihood of time to progression and longest survival; and
optionally wherein a subject having a neuronal like malignant expression program or a malignant squamoid expression program has the greatest likelihood of least time to progression.

43. The method of claim 41, wherein the CAF signature and/or program comprises
a. comprises a myofibroblast program; a neurotropic program; a secretory program;
a mesodermal progenitor program a neuromuscular program; or any combination thereof;
b. comprises a neoadjuvant treated CAF signature and/or program selected from: a neuromuscular program, a secretory program, a neurotropic program, or any combination thereof;
c. comprises an untreated CAF signature and/or program selected from: a mesodermal progenitor program, a myofibroblast program, a neurotropic program, a secretory program, or any combination thereof; or
d. comprises an adhesive expression program, an immunomodulatory expression program, a myofibroblastic progenitor expression program, or a neurotropic expression program
optionally wherein a subject having an immunomodulatory CAF expression program has the greatest likelihood of time to progression; and
optionally wherein a subject having an adhesive CAF expression program has the greatest likelihood of shortest survival.

44. The method of claim 41, wherein the method comprises, detecting, in one or more a PDAC tumor cells, an untreated tumor malignant cell signature and/or program and an untreated CAF signature and/or program, wherein
a. the untreated tumor malignant cell signature and/or program comprises a lineage specific expression program selected from: a squamous program, a mesenchymal cytoskeletal program, mesenchymal matrisomal program, a classical progenitor program, a classical activated program, or any combination thereof; and
b. the untreated tumor CAF signature and/or program is selected from: a mesodermal progenitor program, a myofibroblast program, a neurotropic program, a secretory program, or any combination thereof,
optionally further comprising determining a tumor heterogeneity score for the PDAC tumor, wherein the tumor heterogeneity score is calculated by determining a number of highly expressed programs in the one or more PDAC cells;
optionally further comprising assigning the PDAC tumor to a single malignant class and to a single CAF class, wherein the malignant class is selected from A0, A1, A2, S0, S1, S2, C0, C1, C2, M0, M1, M2, P0, P1, or P2, and wherein the CAF class is selected from S0, S1, N0, N1, M0, M1, P0, or P1;
optionally further comprising assigning the PDAC tumor to a single malignant class and to a single CAF class, wherein the malignant class is selected from A0, A1, A2, S0, S1, S2, C0, C1, C2, M0, M1, M2, P0, P1, or P2, and wherein the CAF class is selected from S0, S1, N0, N1, M0, M1, P0, or P1
optionally wherein the PDAC tumor is assigned to a combined risk class that integrates the malignant risk group and CAF risk group class and is selected from: a low combined risk group, a low-intermediate combined risk group, a high-intermediate risk group, or a high combined risk group, wherein:
a. a PDAC tumor in a low malignant risk group and in a low CAF risk group is classified into the low combined risk group;
b. a PDAC tumor in a high malignant risk and in a high CAF risk is classified into the high combined risk group;
c. a PDAC tumor in an intermediate malignant risk group or in an intermediate CAF risk and in a high malignant risk or in a high CAF risk is classified into the high-intermediate combined risk group; and
d. a PDAC tumor in a low malignant risk group and in a high CAF risk group, a PDAC tumor in a high malignant risk group and in a low CAF risk group, a PDAC tumor in a low malignant risk group and in a low CAF risk group, a PDAC tumor in an intermediate malignant risk group and in an intermediate CAF risk group, a PDAC tumor in a low malignant risk group and in an intermediate CAF risk group is classified into the low-intermediate combined risk group;
optionally wherein a subject with a PDAC tumor in low combined risk group has the greatest likelihood of longest survival.

45.-52. (canceled)

53. The method of claim 41, wherein the tumor spatial neighborhood is
a. a treatment enriched neighborhood
b. a squamoid-basaloid neighborhood; or a
c. a classical neighborhood.

54. The method of claim 41, wherein the one or more co-expressed receptor-ligand pairs is selected from:
a. an Epithelial compartment—CAF compartment pair;
b. an Epithelial compartment—Immune compartment pair;
c. a CAF compartment and Immune compartment pair;
d. or any combination thereof.

55. The method of claim 41, wherein the malignant cell signature comprises one or more biomarkers, expression programs, biologic programs, receptor-ligand interactions, cell state distribution, cell type distribution, or any combination thereof as in any of 1B-1D, 2A-2D, 3A-3C, 3E, 5, 4B-4D, 5A-5C, 6A-6B, 7, 10, 11, 12, 16B-16E, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3, 4, and any combination thereof.

56. The method of claim 41, wherein the CAF cell signature one or more biomarkers, expression programs, biologic programs, receptor-ligand interactions, cell state distribution, cell type distribution, or any combination thereof FIGS. 1B-1D, 2A-2D, 3A-3B, 3E, 5A-5C, 6A-6B, 7, 9C-9D, 14, 15A-15D, 16B, 17B-17G, 18A-18D, 19A-19D, 20A-20C, 21A-21B, 23, 24, 25, 26, 26, 29, 30, 31, 32, 36, 37, 38, 39, and Tables 2.1-2.6, 3 or 5.

57. The method of claim 41, wherein the immune microniche signature one or more biomarkers, expression programs, biologic programs, receptor-ligand interactions, cell state distribution, cell type distribution, or any combination thereof as in any of FIGS. 1B-1D, 2A-2D, 4A-4F, 6A-6B, 9A-9B, 12, 17B-17G, 18A-18D, 19A-19D, 20A-20C, 21A-21B, 23, 24, 25, 26, 26, 29, 30, 31, 32, 36, 37, 38, 39, and Table 7, or any combination thereof.

58. The method of claim 41, wherein the PDAC treatment comprises a neoadjuvant therapy.

59. The method of claim 41, wherein the PDAC treatment comprises
a. preventing a shift in the state of a malignant cell from a classical progenitor state to a basal-like state or a terminally-differentiated state;
b. modulating a cell state of a malignant cell from a basal-like state or a terminally-differentiated state to a classical progenitor state;
c. inhibiting, preventing, or modulating expression of a neuronal like expression program in a malignant cells;
d. inhibiting, preventing expression or modulating expression of a malignant squamoid expression program in a malignant cell;
e. inhibiting, preventing, or modulating expression of an adhesive CAF expression program in a CAF cell; or
f. any combination thereof.

60. The method of claim 41, wherein the PDAC treatment is a PDAC signature modulating agent.

61. The method of claim 41, wherein the PDAC modulating agent is selected by performing a method as in any one of claims 37-39.

62. The method of claim 41, wherein the subject
a. has had neoadjuvant therapy.
b. is concurrently receiving or undergoing neoadjuvant therapy; or
c. the subject has not had neoadjuvant therapy.

63. The method of claim 41, wherein the subject has had a PDAC tumor resected prior to administration.

64. The method of claim 41, wherein the subject has not had a PDAC tumor resected prior to administration.
